(19) United States
(12) Patent Application Publication
(10) Pub. No.: US 2010/0190747 A1
(43) Pub. Date: Jul. 29, 2010

(54) Fused ring compound and use thereof

(76) Inventors: Hideo Suzuki, Osaka (JP); Takuya Fujimoto, Osaka (JP); Takeshi Yamamoto, Osaka (JP)

Correspondence Address: WENDEROTH, LIND & PONACK, L.L.P., 1030 15th Street, N.W.,, Suite 400 East, Washington, DC, 20005-1503, US

(21) Appl. No.: 12/656,332

(22) Filed: Jan. 26, 2010

(30) Foreign Application Priority Data
Jan. 27, 2009 (JP) 2009-15832

(51) Int. Cl. A61K 31/695; C07D 487/04; A61K 31/519; C07D 413/12; A61K 31/5377; C07D 417/12; A61K 31/541; A61P 3/10; A61P 3/04

(52) U.S. Cl. 514/63; 544/280; 514/265.1; 544/117; 514/234.2; 544/58.2; 514/228.5

(57) ABSTRACT

The present invention provides a compound represented by the formula:

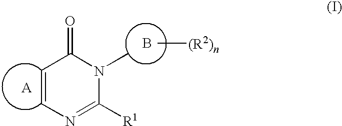

wherein the symbols are as described in the specification, or a salt thereof, which is useful for preventing/treating eicosanoid-associated diseases such as atherosclerosis, diabetes, obesity, atherothrombosis, asthma, fever, pain, cancer, rheumatism, osteoarthritis and atopic dermatitis, and which has an excellent pharmacological action, physicochemical properties, etc.

### TECHNICAL FIELD

The present invention relates to a novel condensed ring compound having an excellent property as a medical drug, a method for producing the compound and use of the compound. More particularly, the present invention relates to a condensed ring compound with a specific structure that inhibits delta-5-desaturase, that has various pharmacological effects based on suppression of eicosanoid production, that has excellent properties such as favorable crystallinity and stability, and that is useful as a prophylactic/therapeutic agent for eicosanoid-related diseases such as atherosclerosis, atherothrombosis, diabetes, obesity, asthma, fever, pain, cancer, rheumatism, osteoarthritis or atopic dermatitis, a salt thereof or a prodrug thereof, a method for producing the compound, a salt thereof or a prodrug thereof, and use of the compound a salt thereof or a prodrug thereof.

### BACKGROUND OF THE INVENTION

Eicosanoids such as prostaglandin, leukotriene and thromboxane appear to play an important role in various diseases. For example, an inflammatory eicosanoid production pathway is considered to be activated in inflammatory diseases such as atherosclerosis, diabetes, obesity, asthma, rheumatism, osteoarthritis and inflammatory pain, and involved in onset and exacerbation of these diseases.

Agents that suppress the eicosanoid signaling such as cyclooxygenase inhibitors and thromboxane A2 receptor antagonists are clinically applied as therapeutic agents for eicosanoid-related diseases. Needs for dealing with inflammatory diseases, however, are still high, and development of potent therapeutic drugs with fewer side-effects has been longed for.

To date, compounds that inhibit delta-5-desaturase have been reported, for example, in WO2008/089307, WO2008/089310 and the like.

WO2008/089307 discloses that compounds such as a compound represented by the following formula has an inhibitory effect on delta-5-desaturase and applications for preventing or treating pain, inflammation, cancer, and ocular diseases and disorders:

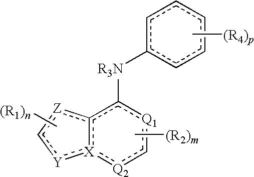

(wherein, X is CH or N; Y is O, S, CR₁, CHR₁, N, or NR₁; Z is O, S, CR₁, CHR₁, N, or NR₁; Q₁ is CR₂, CHR₂, N, or NR₂; Q₂ is CR₂, CHR₂, N, or NR₂; Each R₁ is independently OR_{1A}, N(R_{1A})₂, NC(O)R_{1A}, hydrogen or the like; each R_{1A} is independently hydrogen or optionally substituted alkyl or the like; each R₂ is independently OR_{2A}, N(R_{2A})₂, NC(O)R_{2A}, hydrogen, cyano, nitro, halo, or optionally substituted alkyl, aryl, alkylaryl, arylalkyl or the like; each R_{2A} is independently hydrogen or optionally substituted alkyl or the like; R₃ is independently hydrogen or optionally substituted alkyl; each R₄ is independently OR_{4A}, N(R_{4A})₂, NC(O)R_{4A}, hydrogen, cyano or the like; each R_{4A} is independently hydrogen or optionally substituted alkyl, aryl or the like; n is 1-3; m is 1-3; and p is 1-5).

WO2008/089310 discloses that the compounds of the above formula have an inhibitory effect on delta-5-desaturase and applications for preventing or treating body composition disorders.

Meanwhile, WO2007/002701 discloses that compounds such as a compound represented by the following formula has an application for treating diseases such as inflammatory and immune conditions and diseases mediated by CXCR3 chemokine receptor:

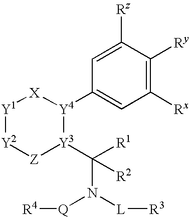

(wherein, X is a member selected from the group consisting of a bond, —C(O)—, C(R⁵)(R⁶)— or the like; Z is a member selected from the group consisting of a bond, —N═, —O—, —S—, —C(R⁷)═, and —N(R¹⁴)—, with the proviso that X and Z are not both a bond; L is a member selected from the group consisting of a bond, C(O)—(C₁-C₈)alkylene or the like; Q is a member selected from the group consisting of (C₁-C₈)alkylene or the like; R¹ and R² are members independently a member selected from the group consisting of H, (C₁-C₈)alkyl or the like; R³ is absent or is a member selected from the group consisting of hydrogen, hydroxy or the like; R⁴ is a member selected from the group consisting of (C₂-C₂₀)alkyl or the like; R⁵ and R⁶ are each members independently selected from the group consisting of H, (C₁-C₈)alkyl or the like; R⁷ and R⁸ are each members independently selected from the group consisting of H, (C₁-C₈)alkyl or the like; each R⁹, R¹⁰, R¹¹ is independently selected from the group consisting of H, (C₁-C₈)alkyl or the like; R^{x}, R^{y}, and R^{z} are each independently H, F or cyano, wherein at least one of R^{x}, R^{y}, and R^{z} is cyano; Y¹ and Y² are each members independently selected from the group consisting of —C(R¹²)═, —CH(R¹²)—, —N═ or the like; Y³ is N or C wherein when Y³ is C, Y³ shares a double bond with Y², Y⁴ or Z; and Y⁴ is N or C wherein when Y⁴ is C, Y⁴ shares a double bond with X, Y¹ or Y³; each R¹² is a member selected from the group consisting of H, halogen, hydroxyl, amino, alkylamino, dialkylamino, (C₁-C₈)alkyl, cyclo(C₃-C₆)alkyl or the like; optionally when Y¹ and Y² are each one of —C(R¹²)═ or —CH(R¹²)—, the two R¹² groups can be combined to form a substituted or unsubstituted 5- to 6-membered cycloalkyl, cycloheteroalkyl, aryl or heteroaryl ring).

A compound represented by the following formula is described in Journal of Combinatorial Chemistry, 2005, 7(6), p. 977-986:

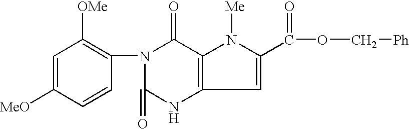

A compound represented by the following formula is described in Journal of Combinatorial Chemistry, 2005, 7(4), p. 589-598:

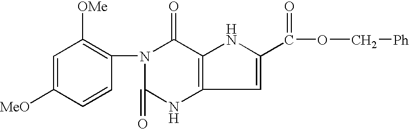

A compound represented by the following formula is described in Indian Journal of Chemistry, Sec. B, Organic Chemistry Including Medicinal Chemistry, 2000, 39B(10), p. 764-768:

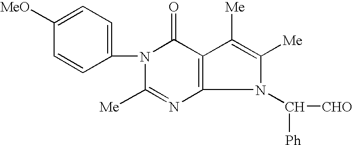

A compound represented by the following formula is described in Journal of the Chinese Chemical Society, 1992, 39(1), p. 101-104 and Archives of pharmacal research, 1990, 13(1), p. 97-100:

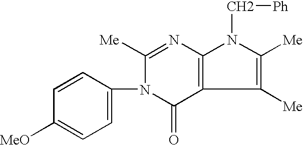

A compound represented by the following formula is described in Heterocycles, 1990, 31(2), p. 367-372:

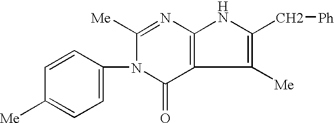

A compound represented by the following formula is described in Chemica Scripta, 1988, 28(3), p. 303-305:

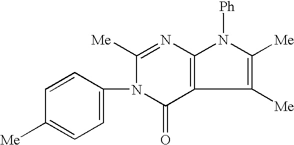

A compound represented by the following formula is described in Heterocycles, 1986, 24(4), p. 997-1006:

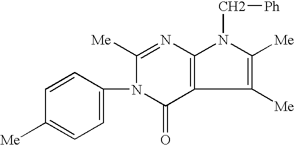

### Problems to be Solved by the Invention

An objective of the present invention is to provide a compound that is useful for preventing/treating eicosanoid-related diseases such as atherosclerosis, atherothrombosis, diabetes, obesity, asthma, fever, pain, cancer, rheumatism, osteoarthritis and atopic dermatitis, and that has excellent pharmacological effects and physicochemical properties.

### Means for Solving the Problems

We found for the first time that a condensed ring compound represented by the following general formula (I) inhibits delta-5-desaturase, shows various pharmacological effects based on suppression of eicosanoid production, has excellent properties such as favorable crystallinity and stability, and is useful for preventing/treating eicosanoid-related diseases such as atherosclerosis, atherothrombosis, diabetes, obesity, asthma, fever, pain, cancer, rheumatism, osteoarthritis or atopic dermatitis. We accomplished the present invention based on this finding and as a result of intensive studies.

That is, this invention relates to
[1] A compound represented by the formula (I):

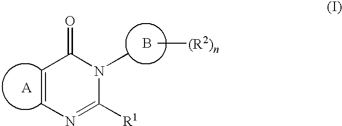

wherein:

R¹ is a hydrogen atom, a substituted or unsubstituted C₁₋₆ alkyl, a substituted or unsubstituted C₃₋₈ cycloalkyl, a substituted or unsubstituted amino, —OR′, —SR′, —SOR″ or —SO₂R″ wherein R′ is a hydrogen atom, a substituted or unsubstituted C₁₋₆ alkyl, a substituted or unsubstituted C₃₋₆ cycloalkyl, or a substituted or unsubstituted cyclic group; and R″ is a substituted or unsubstituted C₁₋₆ alkyl, a substituted or unsubstituted C₃₋₆ cycloalkyl, or a substituted or unsubstituted cyclic group;

R² is a hydrogen atom, a halogen atom, a substituted or unsubstituted C₁₋₆ alkyl, or a substituted or unsubstituted C₁₋₆ alkoxy;

n is an integer from 1 to 5;

a condensed ring including Ring A is a ring represented by any of the following formulae:

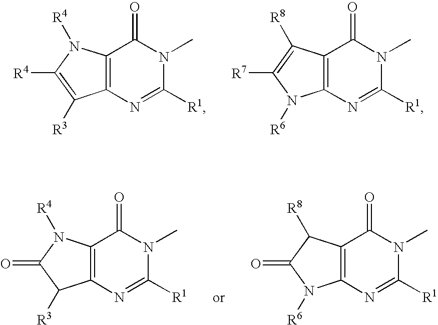

wherein:

R³ is a hydrogen atom, a substituted or unsubstituted C₁₋₆ alkyl, or a substituted or unsubstituted C₃₋₈ cycloalkyl;

R⁴ is a hydrogen atom, a halogen atom, a hydroxy, a substituted or unsubstituted C₁₋₆ alkyl, or a substituted or unsubstituted C₁₋₆ alkoxy;

R⁵ is a hydrogen atom, or a substituted or unsubstituted C₁₋₆ alkyl;

R⁶ is a hydrogen atom, a substituted or unsubstituted C₁₋₆ alkyl, or a substituted or unsubstituted C₃₋₈ cycloalkyl;

R⁷ is a hydrogen atom, a halogen atom, a substituted or unsubstituted hydroxy, a C₂₋₆ alkyl, a substituted C₁₋₆ alkyl, or a substituted or unsubstituted C₁₋₆ alkoxy; and

R⁸ is a hydrogen atom or a halogen atom); and

Ring B is a 5- or 6-membered ring, with proviso that when R⁴ is a hydrogen atom, a halogen atom, a substituted or unsubstituted C₁₋₆ alkyl or a substituted or unsubstituted C₁₋₆ alkoxy, or when R⁷ is a hydrogen atom, a halogen atom, a substituted hydroxy, a C₂₋₆ alkyl, a substituted C₁₋₆ alkyl or a substituted or unsubstituted C₁₋₆ alkoxy, Ring B is a ring represented by the formula:

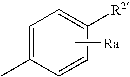

wherein:

R^{2′} is a substituted or unsubstituted C₁₋₆ alkyl or a substituted or unsubstituted C₁₋₆ alkoxy; and

Ra is a hydrogen atom, a halogen atom, a substituted or unsubstituted C₁₋₆ alkyl, or a substituted or unsubstituted C₁₋₆ alkoxy, or a salt thereof (hereinafter also referred to as “Compound (I)”);
[2] The compound according to the above [1], wherein the compound is represented by the formula (I):

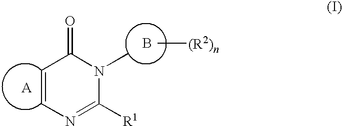

wherein:

R¹ is a hydrogen atom, a substituted or unsubstituted C₁₋₆ alkyl, a substituted or unsubstituted C₃₋₈ cycloalkyl, a substituted or unsubstituted amino, —OR′, —SR′, —SOR″ or —SO₂R″ wherein R′ is a hydrogen atom, a substituted or unsubstituted C₁₋₆ alkyl, a substituted or unsubstituted C₃₋₆ cycloalkyl, or a substituted or unsubstituted cyclic group; and R″ is a substituted or unsubstituted C₁₋₆ alkyl, a substituted or unsubstituted C₃₋₆ cycloalkyl, or a substituted or unsubstituted cyclic group;

R² is a hydrogen atom, a halogen atom, a substituted or unsubstituted C₁₋₆ alkyl, or a substituted or unsubstituted C₁₋₆ alkoxy;

n is an integer from 1 to 5;

a condensed ring including Ring A is a ring represented by any of the following formulae:

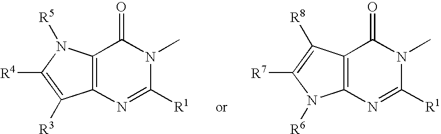

wherein:

R³ is a hydrogen atom, a substituted or unsubstituted C₁₋₆ alkyl, or a substituted or unsubstituted C₃₋₈ cycloalkyl;

R⁴ is a hydrogen atom, a halogen atom, a hydroxy, a substituted or unsubstituted C₁₋₆ alkyl, or a substituted or unsubstituted C₁₋₆ alkoxy;

R⁵ is a hydrogen atom or a substituted or unsubstituted C₁₋₆ alkyl;

R⁶ is a hydrogen atom, a substituted or unsubstituted C₁₋₆ alkyl, or a substituted or unsubstituted C₃₋₈ cycloalkyl;

R⁷ is a hydrogen atom, a halogen atom, a substituted or unsubstituted hydroxy, a C₂₋₆ alkyl, a substituted C₁₋₆ alkyl, or a substituted or unsubstituted C₁₋₆ alkoxy; and

R⁸ is a hydrogen atom or a halogen atom; and

Ring B is a 5- or 6-membered ring, with proviso that when R⁴ is a hydrogen atom, a halogen atom, a substituted or unsubstituted C₁₋₆ alkyl or a substituted or unsubstituted C₁₋₆ alkoxy, or when R⁷ is a hydrogen atom, a halogen atom, a substituted hydroxy, a C₂₋₆ alkyl, a substituted C₁₋₆ alkyl or a substituted or unsubstituted C₁₋₆ alkoxy, Ring B is a ring represented by the formula:

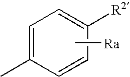

wherein:

R^{2′} is a substituted or unsubstituted C₁₋₆ alkyl or a substituted or unsubstituted C₁₋₆ alkoxy; and

Ra is a hydrogen atom, a halogen atom, a substituted or unsubstituted C₁₋₆ alkyl, or a substituted or unsubstituted C₁₋₆ alkoxy;
[3] The compound according to the above [1] or [2], wherein Ring B is a ring represented by the formula:

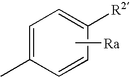

wherein R^{2′} and Ra have the same meanings as those in the above [1];
[4] The compound according to the above [1] or [2], wherein the compound is represented by the formula (I):

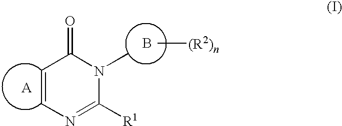

wherein:

R¹ is a hydrogen atom, a substituted or unsubstituted C₁₋₆ alkyl, a substituted or unsubstituted C₃₋₈ cycloalkyl, a substituted or unsubstituted amino, —OR′, —SR′, —SOR″ or —SO₂R″ wherein R′ is a hydrogen atom, a substituted or unsubstituted C₁₋₆ alkyl, a substituted or unsubstituted C₃₋₆ cycloalkyl, or a substituted or unsubstituted cyclic group; and R″ is a substituted or unsubstituted C₁₋₆ alkyl, a substituted or unsubstituted C₃₋₆ cycloalkyl, or a substituted or unsubstituted cyclic group;

R² is a hydrogen atom, a halogen atom, a substituted or unsubstituted C₁₋₆ alkyl, or a substituted or unsubstituted C₁₋₆ alkoxy;

n is an integer from 1 to 5;

a condensed ring including Ring A is a ring represented by any of the following formulae:

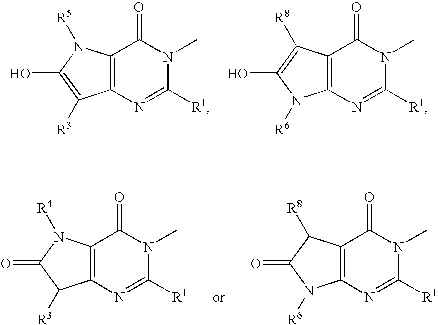

wherein:

R³ is a hydrogen atom, a substituted or unsubstituted C₁₋₆ alkyl, or a substituted or unsubstituted C₃₋₈ cycloalkyl;

R⁵ is a hydrogen atom or a substituted or unsubstituted C₁₋₆ alkyl;

R⁶ is a hydrogen atom, a substituted or unsubstituted C₁₋₆ alkyl, or a substituted or unsubstituted C₃₋₈ cycloalkyl; and

R⁸ is a hydrogen atom or a halogen atom; and

Ring B is a 5- or 6-membered ring;
[5] The compound according to the above [1], [2] or [4], wherein Ring B is a ring represented by the formula:

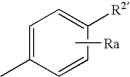

wherein:

R^{2′} is a C₁₋₆ alkoxy which may be substituted with 1 to 9 substituents selected from the group consisting of a halogen atom and a C₃₋₆ cycloalkyl; and

Ra is a hydrogen atom or a halogen atom;
[5A] The compound according to the above [2] or [3], wherein R¹ is —OR′ or —SR′ wherein R′ has the same meaning as that in the above [2];
[6] The compound according to the above [2], [3], [4] or [5], wherein R¹ is —OR′ or —SR′ wherein R′ is a C₁₋₆ alkyl, a C₃₋₆ cycloalkyl or a C₆₋₁₄ aryl, each of which may be substituted with 1 to 5 substituents selected from the group consisting of (a) a halogen atom, (b) a C₁₋₆ alkoxy which may be substituted with 1 to 3 C₁₋₆ alkoxy, (c) a C₃₋₆ cycloalkyl and (d) a C₁₋₆ alkylsulfonyl;
[7] The compound according to the above [2], [3], [4], [5] or [6], wherein R² is (a) a hydrogen atom, (b) a halogen atom or (c) a C₁₋₆ alkoxy which may be substituted with 1 to 9 substituents selected from the group consisting of a halogen atom and a C₃₋₆ cycloalkyl; and n is 1;
[7A] The compound according to the above [2], [3] or [5A], wherein the condensed ring including Ring A is represented by the formula:

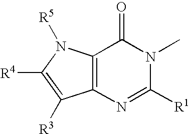

wherein R¹, R³, R⁴ and R⁵ have the same meanings as those in the above [2];
[8] The compound according to the above [2], [3], [4], [5], [6] or [7]wherein the condensed ring including Ring A is a ring represented by any of the following formulae:

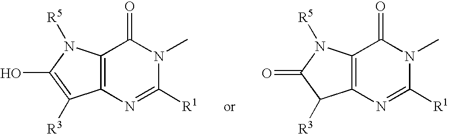

wherein R¹, R³ and R⁵ have the same meanings as those in the above [4];
[9] The compound according to the above [2], [3], [4], [5], [6], [7] or [8], wherein R³ is a hydrogen atom, a C₁₋₆ alkyl or a C₃₋₈ cycloalkyl;
[10] The compound according to the above [2], [3], [4], [5], [6], [7], [8] or [9], wherein R⁵ is a hydrogen atom;
[10A] The compound according to the above [2], [3] or [5A], wherein the condensed ring including Ring A is a ring represented by the formula:

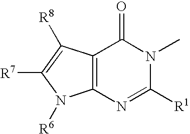

wherein R¹, R⁶, R⁷ and R⁸ have the same meanings as those in the above [2];
[11] The compound according to the above [2], [3], [4], [5], [6] or [7], wherein the condensed ring including Ring A is a ring represented by any of the following formulae:

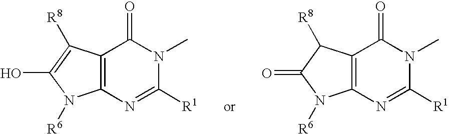

wherein R¹, R⁶ and R⁸ have the same meanings as those in the above [4];
[12] The compound according to the above [2], [3], [4], [5], [6], [7] or [11], wherein R⁶ is a hydrogen atom or a substituted or unsubstituted C₁₋₆ alkyl;
[13] The compound according to the above [2], [3], [4], [5], [6], [7], [11] or [12], wherein R⁸ is a hydrogen atom;
[14] The compound according to the above [4], wherein:

R¹ is —OR′ or —SR′ wherein R′ is a C₁₋₆ alkyl, a C₃₋₆ cycloalkyl or a C₆₋₁₄ aryl, each of which may be substituted with 1 to 5 substituents selected from the group consisting of (a) a halogen atom, (b) a C₁₋₆ alkoxy which may be substituted with 1 to 3 C₁₋₆ alkoxy, (c) a C₃₋₆ cycloalkyl and (d) a C₁₋₆ alkylsulfonyl;

the condensed ring including Ring A is a ring represented by any of the following formulae:

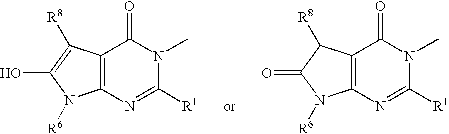

wherein:

R⁶ is a hydrogen atom, or a C₁₋₆ alkyl which may be substituted with 1 to 3 C₁₋₆ alkoxy; and

R⁸ is a hydrogen atom or a halogen atom; and

Ring B is a ring represented by the formula:

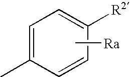

wherein:

R^{2′} is a C₁₋₆ alkoxy which may be substituted with 1 to 9 substituents selected from the group consisting of a halogen atom and a C₃₋₆ cycloalkyl; and

Ra is a hydrogen atom or a halogen atom;
[14A] The compound according to the above [2], wherein the compound is represented by the formula:

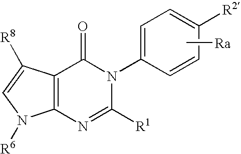

wherein:

R¹ is —OR′ or —SR′ wherein R′ is a C₁₋₆ alkyl, a C₃₋₆ cycloalkyl or a C₆₋₁₄ aryl, each of which may be substituted with 1 to 5 substituents selected from the group consisting of (a) a halogen atom, (b) a C₁₋₆ alkoxy which may be substituted with 1 to 3 C₁₋₆ alkoxy, (c) a C₃₋₆ cycloalkyl and (d) a C₁₋₆ alkylsulfonyl;

R^{2′} is a C₁₋₆ alkoxy which may be substituted with 1 to 9 substituents selected from the group consisting of a halogen atom and a C₃₋₆ cycloalkyl;

Ra is a hydrogen atom or a halogen atom;

R⁶ is a hydrogen atom, or a C₁₋₆ alkyl which may be substituted with 1 to 3 C₁₋₆ alkoxy; and

R⁸ is a hydrogen atom or a halogen atom];
[15] 2-(2,2,2-Trifluoroethoxy)-3-[4-(2,2,2-trifluoroethoxy)phenyl]-5,7-dihydro-3H-pyrrolo[2,3-d]pyrimidine-4,6-dione or a salt thereof;
[16] 2-(2,2,3,3,3-Pentafluoropropoxy)-3-[4-(2,2,2-trifluoroethoxy)phenyl]-5,7-dihydro-3H-pyrrolo[2,3-d]pyrimidine-4,6-dione or a salt thereof;
[17] 2-[(Cyclopropylmethyl)sulfanyl]-3-[4-(2,2,2-trifluoroethoxy)phenyl]-5,7-dihydro-3H-pyrrolo[2,3-d]pyrimidine-4,6-dione or a salt thereof;
[18] 2-(2,2,2-Trifluoroethoxy)-3-[4-(2,2,2-trifluoroethoxy)phenyl]-3,7-dihydro-4H-pyrrolo[2,3-d]pyrimidine-4-one or a salt thereof;
[19] 2-(2,2,3,3,3-Pentafluoropropoxy)-3-[4-(2,2,2-trifluoroethoxy)phenyl]-3,7-dihydro-4H-pyrrolo[2,3-d]pyrimidine-4-one or a salt thereof;
[20] 2-[(Cyclopropylmethyl)sulfanyl]-3-[4-(2,2,2-trifluoroethoxy)phenyl]-3,7-dihydro-4H-pyrrolo[2,3-d]pyrimidine-4-one or a salt thereof;
[21] A prodrug of the compound according to the above [1];
[22] A pharmaceutical composition comprising the compound according to the above [1] or a prodrug thereof;
[23] The pharmaceutical composition according to the above [22], which is a delta-5-desaturase inhibitor;
[24] The pharmaceutical composition according to the above [22], which is a prophylactic or therapeutic agent for eicosanoid-mediated diseases;
[25] The pharmaceutical composition according to the above [22], which is a prophylactic or therapeutic agent for atherosclerosis;
[26] The pharmaceutical composition according to the above [22], which is a prophylactic or therapeutic agent for diabetes or obesity;
[27] A method for preventing or treating atherosclerosis in a mammal, which comprises administering an effective amount of the compound according to the above [1] or a prodrug thereof to the mammal;
[28] A method for preventing or treating diabetes or obesity in a mammal, which comprises administering an effective amount of the compound according to the above [1] or a prodrug thereof to the mammal;
[29] Use of the compound according to the above [1] or a prodrug thereof to manufacture a prophylactic or therapeutic agent for atherosclerosis; and
[30] Use of the compound according to the above [1] or a prodrug thereof to manufacture a prophylactic or therapeutic agent for diabetes or obesity.

### BRIEF DESCRIPTION OF THE DRAWINGS

FIG. 1 shows the powder X-ray crystal diffraction pattern of the crystals obtained in Example 352.

FIG. 2 shows the powder X-ray crystal diffraction pattern of the crystals obtained in Example 353.

### DETAILED DESCRIPTION OF THE INVENTION

Hereinafter, the definitions of symbols used in the specification will be described in detail.

Examples of the “halogen atom” in the specification include a fluorine atom, a chlorine atom, a bromine atom, and an iodine atom.

Examples of the “C₁₋₆ alkyl” in the specification include methyl, ethyl, propyl, isopropyl, butyl, isobutyl, sec-butyl, tert-butyl, pentyl, isopentyl, neopentyl, tert-pentyl, hexyl, 2,2-dimethylbutyl, 3,3-dimethylbutyl, and 2-ethylbutyl.

Examples of the “C₂₋₆ alkyl” in the specification include ethyl, propyl, isopropyl, butyl, isobutyl, sec-butyl, tert-butyl, pentyl, isopentyl, neopentyl, tert-pentyl, hexyl, 2,2-dimethylbutyl, 3,3-dimethylbutyl, and 2-ethylbutyl.

Examples of the “C₂₋₆ alkenyl” in the specification include vinyl, allyl, propenyl, isopropenyl, buta-3-en-1-yl, penta-4-en-1-yl, and hexa-5-en-1-yl.

Examples of the “C₂₋₆ alkynyl” in the specification include ethynyl, prop-2-yn-1-yl, buta-3-yn-1-yl, penta-4-yn-1-yl, and hexa-5-yn-1-yl.

Examples of the “C₃₋₆ cycloalkyl” in the specification include cyclopropyl, cyclobutyl, cyclopentyl, and cyclohexyl.

Examples of the “C₃₋₈ cycloalkyl” in the specification include cyclopropyl, cyclobutyl, cyclopentyl, cyclohexyl, cycloheptyl, and cyclooctyl. Among them, a C₃₋₆ cycloalkyl group is preferred.

Examples of the “C₆₋₁₄ aryl” in the specification include phenyl, naphthyl (e.g., 1-naphthyl and 2-naphthyl), anthryl, and phenanthryl.

Examples of the “C₇₋₁₆ aralkyl” in the specification include benzyl, 1-phenylethyl, 2-phenylethyl, naphthylmethyl (1-naphthylmethyl, 2-naphthylmethyl), 3-phenylpropyl, 4-phenylbutyl, and 5-phenylpentyl.

Examples of the “C₁₋₆ alkoxy” in the specification include methoxy, ethoxy, propoxy, isopropoxy, butoxy, isobutoxy, sec-butoxy, tert-butoxy, pentyloxy, isopentyloxy, neopentyloxy, tert-pentyloxy, hexyloxy, and 2-ethylbutoxy.

Examples of the “heterocyclic group” in the specification are an aromatic heterocyclic group and a non-aromatic heterocyclic group, unless otherwise specified.

In this regard, examples of the “aromatic heterocyclic group” include a 5- to 7-membered (preferably 5- or 6-membered) monocyclic aromatic heterocyclic group containing 1 to 4 hetero atoms selected from an oxygen atom, a sulfur atom and a nitrogen atom as ring-constituting atoms other than a carbon atom, and a condensed aromatic heterocyclic group. Examples of the condensed aromatic heterocyclic group include a group derived from a ring formed by condensation of a ring corresponding to the 5- to 7-membered monocyclic aromatic heterocyclic group and 1 or 2 rings selected from a 5- or 6-membered aromatic heterocyclic ring containing 1 or 2 nitrogen atoms (e.g., pyrrole, imidazole, pyrazole, pyrazine, pyridine and pyrimidine), a 5-membered aromatic heterocyclic ring containing a sulfur atom (e.g., thiophene) and a benzene ring.

Examples of the “aromatic heterocyclic group” include: monocyclic aromatic heterocyclic rings such as furyl, thienyl, pyrrolyl, oxazolyl, isoxazolyl, thiazolyl, isothiazolyl, imidazolyl, pyrazolyl, 1,2,3-oxadiazolyl, 1,2,4-oxadiazolyl, 1,3,4-oxadiazolyl, furazanyl, 1,2,3-thiadiazolyl, 1,2,4-thiadiazolyl, 1,3,4-thiadiazolyl, 1,2,3-triazolyl, 1,2,4-triazolyl, tetrazolyl, pyridyl, pyridazinyl, pyrimidinyl, pyrazinyl, and triazinyl; and condensed aromatic heterocyclic rings such as benzofuranyl, isobenzofuranyl, benzo[b]thienyl, indolyl, isoindolyl, 1H-indazolyl, benzoimidazolyl, benzooxazolyl, benzo[d]isoxazolyl, benzothiazolyl, benzo[d]isothiazolyl, 1H-benzotriazolyl, quinolyl, isoquinolyl, cinnolinyl, quinazolinyl, quinoxalinyl, phthalazinyl, naphthyridinyl, purinyl, pteridinyl, carbazolyl, α-carbolinyl, β-carbolinyl, γ-carbolinyl, acridinyl, phenoxazinyl, phenothiazinyl, phenazinyl, phenoxathiinyl, thianthrenyl, phenathridinyl, phenathridinyl, phenanthrolinyl, indolydinyl, pyrrolo[1,2-b]pyridazinyl, pyrazolo[1,5-a]pyridyl, imidazo[1,2-a]pyridyl, imidazo[1,5-a]pyridyl, imidazo[1,2-a]pyridazinyl, imidazo[1,2-a]pyrimidinyl, 1,2,4-triazolo[4,3-a]pyridyl and 1,2,4-triazolo[4,3-b]pyridazinyl.

Examples of the non-aromatic heterocyclic group include a 4- to 7-membered (preferably 5- or 6-membered) monocyclic non-aromatic heterocyclic group containing 1 to 4 hetero atoms selected from an oxygen atom, a sulfur atom and a nitrogen atom as ring-constituting atoms other than a carbon atom, and a condensed non-aromatic heterocyclic group. Examples of the condensed non-aromatic heterocyclic group include a group derived from a ring formed by condensation of a ring corresponding to the 4- to 7-membered monocyclic non-aromatic heterocyclic group and 1 or 2 rings selected from a 5- or 6-membered aromatic heterocyclic ring containing 1 or 2 nitrogen atoms (e.g., pyrrole, imidazole, pyrazole, pyrazine, pyridine and pyrimidine), a 5-membered aromatic heterocyclic ring containing a sulfur atom (e.g., thiophene) and a benzene ring, and a group obtained by partial saturation of the group.

Examples of the “non-aromatic heterocyclic group” include: monocyclic non-aromatic heterocyclic rings such as azetidinyl, oxetanyl, thiethanyl, pyrrolidinyl, tetrahydrofuryl, thiolanyl, imidazolidinyl, pyrazolidinyl, oxazolidinyl, thiazolidinyl, piperidyl, tetrahydropyranyl, morpholinyl, thiomorpholinyl and piperazinyl; and condensed non-aromatic heterocyclic groups such as isochromanyl, dihydrobenzopyranyl, isochromenyl, chromenyl(2H-chromenyl, 4H-chromenyl), 1,2,3,4-tetrahydroisoquinolyl, 1,2,3,4-tetrahydroquinolyl, 2,3-dihydrobenzofuranyl and benzo[1,3]dioxolyl.

Examples of the “C₃₋₆ cycloalkyloxy” in the specification include cyclopropyloxy, cyclobutyloxy, cyclopentyloxy, and cyclohexyloxy.

Examples of the “C₆₋₁₄ aryloxy” in the specification include phenoxy, 1-naphthyloxy, and 2-naphthyloxy.

Examples of the “C₇₋₁₆ aralkyloxy” in the specification include benzyloxy and phenethyloxy.

Examples of the “C₁₋₆ alkylamino” in the specification include amino monosubstituted with the above-described “C₁₋₆ alkyl”. Specific examples thereof include methylamino, ethylamino, propylamino, isopropylamino, butylamino, isobutylamino, sec-butylamino, tert-butylamino, pentylamino, isopentylamino, neopentylamino, tert-pentylamino, and hexylamino.

Examples of the “di C₁₋₆ alkylamino” in the specification include amino disubstituted with the above-described “C₁₋₆ alkyl”. Specific examples thereof include dimethylamino, diethylamino, and N-ethyl-N-methylamino.

Examples of the “C₆₋₁₄ arylamino” in the specification include amino monosubstituted with the above-described “C₆₋₁₄ aryl”. Specific examples thereof include phenylamino, 1-naphthylamino, and 2-naphthylamino.

Examples of the “di C₆₋₁₄ arylamino” in the specification include amino disubstituted with the above-described “C₆₋₁₄ aryl”. Specific examples thereof include diphenylamino and dinaphthylamino.

Examples of the “C₇₋₁₆ aralkylamino” in the specification include amino monosubstituted with the above-described “C₇₋₁₆ aralkyl”. Specific examples thereof include benzylamino and phenethylamino.

Examples of the “di C₇₋₁₆ aralkylamino” in the specification include amino disubstituted with the above-described “C₇₋₁₆ aralkyl”. Specific examples thereof include dibenzylamino and diphenethylamino.

Examples of the “N—C₁₋₆ alkyl-N—C₆₋₁₄ arylamino” in the specification include amino substituted with the above-described “C₁₋₆ alkyl” and the above-described “C₆₋₁₄ aryl”. Examples thereof include N-methyl-N-phenylamino and N-ethyl-N-phenylamino.

Examples of the “N—C₁₋₆ alkyl-N—C₇₋₁₆ aralkylamino” in the specification include amino substituted with the above-described “C₁₋₆ alkyl” and the above-described “C₇₋₁₆ aralkyl”. Examples thereof include N-methyl-N-benzylamino and N-ethyl-N-benzylamino.

Examples of the “C₁₋₆ alkyl-carbonylamino” in the specification include acetylamino, propanoylamino, butanoylamino, 2-methylpropanoylamino, pentanoylamino, 3-methylbutanoylamino, and 2,2-dimethylpropanoylamino.

Examples of the “C₁₋₆ alkylthio” in the specification include methylthio, ethylthio, propylthio, isopropylthio, butylthio, sec-butylthio, and tert-butylthio.

Examples of the “C₁₋₆ alkylsulfinyl” in the specification include methylsulfinyl, ethylsulfinyl, propylsulfinyl, isopropylsulfinyl, butylsulfinyl, sec-butylsulfinyl, and tert-butylsulfinyl.

Examples of the “C₁₋₁₆ alkylsulfonyl” in the specification include methylsulfonyl, ethylsulfonyl, propylsulfonyl, isopropylsulfonyl, butylsulfonyl, sec-butylsulfonyl, and tert-butylsulfonyl.

Examples of the “C₁₋₆ alkylsulfonyloxy” in the specification include methylsulfonyloxy, ethylsulfonyloxy, propylsulfonyloxy, isopropylsulfonyloxy, butylsulfonyloxy, sec-butylsulfonyloxy, and tert-butylsulfonyloxy.

Examples of the “carboxy which may be esterified” in the specification include:
(1) carboxy;
(2) C₁₋₆ alkoxy-carbonyl (e.g., methoxycarbonyl, ethoxycarbonyl, propoxycarbonyl and tert-butoxycarbonyl);
(3) C₆₋₁₄ aryloxy-carbonyl (e.g., phenoxycarbonyl); and
(4) C₇₋₁₆ aralkyloxy-carbonyl (e.g., benzyloxycarbonyl and phenethyloxycarbonyl).

Examples of the “C₁₋₆ alkyl-carbonyl” in the specification include acetyl, propanoyl, butanoyl, 2-methylpropanoyl, pentanoyl, 3-methylbutanoyl, and 2,2-dimethylpropanoyl.

Examples of the “C₁₋₆ alkyl-carbonyloxy” in the specification include acetyloxy, propanoyloxy, butanoyloxy, 2-methylpropanoyloxy, pentanoyloxy, 3-methylbutanoyloxy, and 2,2-dimethylpropanoyloxy.

Examples of the “C₃₋₁₀ cycloalkyl-carbonyl” in the specification include cyclopentylcarbonyl, cyclohexylcarbonyl, and adamantylcarbonyl.

Examples of the “C₆₋₁₄ aryl-carbonyl” in the specification include benzoyl, 1-naphthoyl, and 2-naphthoyl.

Examples of the “C₇₋₁₆ aralkyl-carbonyl” in the specification include phenylacetyl and 3-phenylpropanoyl.

Examples of the “C₁₋₆ alkoxy-carbonyl” in the specification include methoxycarbonyl, ethoxycarbonyl, propoxycarbonyl, and tert-butoxycarbonyl.

Examples of the “C₆₋₁₄ aryloxy-carbonyl” in the specification include phenoxycarbonyl, 1-naphthyloxycarbonyl, and 2-naphthyloxycarbonyl.

Examples of the “C₇₋₁₆ aralkyloxy-carbonyl” in the specification include benzyloxycarbonyl and phenethyloxycarbonyl.

Examples of the “heterocyclic ring” of the “heterocyclic ring-carbonyl” in the specification include the aromatic or non-aromatic heterocyclic group exemplified above as the heterocyclic group. Specific examples of the “heterocyclic ring-carbonyl” include benzofuranylcarbonyl, thienylcarbonyl, benzoimidazolylcarbonyl, pyrimidinylcarbonyl, 1-pyrrolidinylcarbonyl, piperidinocarbonyl, 1-piperazinylcarbonyl, morpholinocarbonyl, and thiomorpholinocarbonyl.

The “heterocyclic ring” of the “heterocyclic ring-carbonyl” may be further substituted with 1 to 3 substituents selected from the group consisting of C₁₋₆ alkyl, halogen and a heterocyclic group.

Examples of the “C₁₋₆ alkyl-carbamoyl” in the specification include carbamoyl monosubstituted with the above-described “C₁₋₆ alkyl”. Specific examples thereof include methylcarbamoyl and ethylcarbamoyl.

Examples of the “di C₁₋₆ alkyl-carbamoyl” in the specification include carbamoyl disubstituted with the above-described “C₁₋₆ alkyl”. Specific examples thereof include dimethylcarbamoyl, diethylcarbamoyl, and N-ethyl-N-methylcarbamoyl.

Examples of the “C₆₋₁₄ aryl-carbamoyl” in the specification include carbamoyl monosubstituted with the above-described “C₆₋₁₄ aryl”. Specific examples thereof include phenylcarbamoyl, 1-naphthylcarbamoyl, and 2-naphthylcarbamoyl.

Examples of the “di C₆₋₁₄ aryl-carbamoyl” in the specification include carbamoyl disubstituted with the above-described “C₆₋₁₄ aryl”. Specific examples thereof include diphenylcarbamoyl and dinaphthylcarbamoyl.

Examples of the “C₁₋₆ alkylsulfamoyl” in the specification include sulfamoyl monosubstituted with the above-described “C₁₋₆ alkyl”. Specific examples thereof include methylsulfamoyl and ethylsulfamoyl.

Examples of the “di C₁₋₆ alkylsulfamoyl” in the specification include sulfamoyl disubstituted with the above-described “C₁₋₆ alkyl”. Specific examples thereof include dimethylsulfamoyl, diethylsulfamoyl, and N-ethyl-N-methylsulfamoyl.

Examples of the “C₃₋₆ cycloalkylsulfamoyl” in the specification include sulfamoyl monosubstituted with the above-described “C₃₋₆ cycloalkyl”. Specific examples thereof include cyclopropylsulfamoyl and cyclobutylsulfamoyl.

Examples of the “C₆₋₁₄ arylsulfamoyl” in the specification include sulfamoyl monosubstituted with the above-described “C₆₋₁₄ aryl”. Specific examples thereof include phenylsulfamoyl, 1-naphthylsulfamoyl, and 2-naphthylsulfamoyl.

Examples of the “di C₆₋₁₄ arylsulfamoyl” in the specification include a sulfamoyl group disubstituted with the above-described “C₆₋₁₄ aryl”. Specific examples thereof include diphenylsulfamoyl and dinaphthylsulfamoyl.

Hereinafter, groups represented by formula (I) will be described.

R¹ means a hydrogen atom, substituted or unsubstituted C₁₋₆ alkyl, substituted or unsubstituted C₃₋₈ cycloalkyl, substituted or unsubstituted amino, —OR′, —SR′, —SOR″ or —SO₂R″ (wherein: R′ is a hydrogen atom, substituted or unsubstituted C₁₋₆ alkyl, substituted or unsubstituted C₃₋₆ cycloalkyl, or a substituted or unsubstituted cyclic group; and R″ is substituted or unsubstituted C₁₋₆ alkyl, substituted or unsubstituted C₃₋₆ cycloalkyl, or a substituted or unsubstituted cyclic group).

Each of the “substituted or unsubstituted C₁₋₆ alkyl”, “substituted or unsubstituted C₃₋₈ cycloalkyl” and “substituted or unsubstituted amino” represented by R¹, the “substituted or unsubstituted C₁₋₆ alkyl” represented by R′, the “substituted or unsubstituted C₃₋₆ cycloalkyl” represented by R′, the “substituted or unsubstituted C₁₋₆ alkyl” represented by R″ and the “substituted or unsubstituted C₃₋₆ cycloalkyl” represented by R″ may have 1 to 9, and preferably 1 to 5 substituents at replaceable positions.

Examples of such substituents include a group (hereinafter also referred to as “Substituent Group A”) consisting of, for example,
(1) a halogen atom,
(2) hydroxy,
(3) amino which may be substituted with 1 to 2 substituents selected from the group consisting of: (i) C₁₋₆ alkyl which may be substituted with cyano; and (ii) C₁₋₆ alkyl-carbonyl which may be substituted with cyano,
(4) nitro,
(5) cyano,
(6) substituted or unsubstituted C₁₋₆ alkyl,
(7) substituted or unsubstituted C₃₋₆ cycloalkyl,
(8) substituted or unsubstituted C₁₋₆ alkoxy,
(9) substituted or unsubstituted imino,
(10) substituted or unsubstituted C₁₋₃ alkylidene,
(11) C₃₋₆ cycloalkyloxy,
(12) C₆₋₁₄ aryloxy which may be substituted with a halogen atom(s),
(13) heterocyclic ring-oxy,
(14) C₇₋₁₆ aralkyloxy,
(15) C₁₋₆ alkylamino,
(16) di C₁₋₆ alkylamino,
(17) C₆₋₁₄ arylamino,
(18) di C₆₋₁₄ arylamino,
(19) C₇₋₁₆ aralkylamino,
(20) di C₇₋₁₆ aralkylamino,
(21) N—C₁₋₆ alkyl-N—C₆₋₁₄ arylamino,
(22) N—C₁₋₆ alkyl-N—C₇₋₁₆ aralkylamino,
(23) C₁₋₆ alkyl-carbonylamino which may be substituted with cyano,
(24) C₁₋₆ alkylthio,
(25) C₁₋₆ alkylsulfinyl,
(26) substituted or unsubstituted C₁₋₆ alkylsulfonyl,
(27) substituted or unsubstituted heterocyclic ring-sulfonyl,
(28) C₁₋₆ alkylsulfonyloxy,
(29) carboxy which may be esterified,
(30) substituted or unsubstituted C₁₋₆ alkyl-carbonyl,
(31) C₁₋₆ alkyl-carbonyloxy,
(32) C₃₋₆ cycloalkyl-carbonyl,
(33) substituted or unsubstituted C₆₋₁₄ aryl-carbonyl,
(34) C₇₋₁₆ aralkyl-carbonyl,
(35) C₁₋₆ alkoxy-carbonyl,
(36) heterocyclic ring-carbonyl which may be substituted with 1 to 3 substituents selected from the group consisting of: (i) hydroxy; (ii) oxo; and (iii) C₁₋₆ alkyl,
(37) carbamoyl which may be substituted with C₃₋₆ cycloalkyl,
(38) thiocarbamoyl,
(39) substituted or unsubstituted C₁₋₆ alkyl-carbamoyl,
(40) substituted or unsubstituted di C₁₋₆ alkyl-carbamoyl,
(41) C₆₋₁₄ aryl-carbamoyl which may be substituted with 1 to 3 C₁₋₆ alkoxy groups,
(42) di C₆₋₁₄ aryl-carbamoyl,
(43) C₁₋₃ alkylidenecarbamoyl,
(44) C₁₋₆ alkylsulfonyl-carbamoyl,
(45) sulfamoyl which may be substituted with C₃₋₆ cycloalkyl-carbonyl,
(46) substituted or unsubstituted C₁₋₆ alkylsulfamoyl,
(47) C₃₋₆ cycloalkylsulfamoyl,
(48) di C₁₋₆ alkylsulfamoyl,
(49) C₆₋₁₄ arylsulfamoyl,
(50) di C₆₋₁₄ arylsulfamoyl,
(51) a substituted or unsubstituted cyclic group, and
(52) silyloxy which may be substituted with 1 to 3 C₁₋₆ alkyl groups.
   When there are 2 or more substituents, they may be the same or different.

Examples of the “substituted or unsubstituted imino” include imino which may be substituted with:
(1) hydroxy; or
(2) C₁₋₆ alkoxy which may be substituted with 1 to 3 substituents selected from the group consisting of
(i) carboxy,
(ii) C₆₋₁₄ aryl (e.g., phenyl),
(iii) C₁₋₆ alkoxy-carbonyl (e.g., ethoxycarbonyl), and
(iv) C₁₋₃ alkylidene (e.g., methylidene)
   (e.g., methoxy, ethoxy and isopropyloxy). When there are 2 or more substituents, they may be the same or different.

Examples of the “C₁₋₃ alkylidene” of the “substituted or unsubstituted C₁₋₃ alkylidene” include methylidene (CH₂═), ethylidene (CH₃CH═) and propylidene (CH₃CH₂CH═).

The “C₁₋₃ alkylidene” may have 1 to 3 substituents at replaceable positions. Examples of such substituents include carboxy which may be esterified. When there are 2 or more substituents, they may be the same or different.

The “substituted or unsubstituted C₁₋₆ alkyl”, “substituted or unsubstituted C₃₋₆ cycloalkyl”, “substituted or unsubstituted C₁₋₆ alkoxy”, “substituted or unsubstituted C₁₋₆ alkylsulfonyl”, “substituted or unsubstituted C₁₋₆ alkyl-carbonyl”, “substituted or unsubstituted C₁₋₆ alkyl-carbamoyl”, “substituted or unsubstituted di C₁₋₆ alkyl-carbamoyl” and “substituted or unsubstituted C₁₋₆ alkylsulfamoyl” may have 1 to 5, and preferably 1 to 3 substituents at replaceable positions.

Examples of such substituents include
(1) a halogen atom,
(2) hydroxy,
(3) C₁₋₆ alkoxy which may be substituted with 1 to 3 substituents selected from the group consisting of:

(i) a halogen atom (e.g., a fluorine atom);

(ii) hydroxy;

(iii) a C₃₋₆ cycloalkyl group (e.g., cyclopropyl); and

(iv) di C₁₋₆ alkylamino group (e.g., dimethylamino),
(4) C₂₋₆ alkynyl,
(5) amino,
(6) cyano,
(7) C₁₋₆ alkylamino,
(8) di C₁₋₆ alkylamino,
(9) C₁₋₆ alkylthio,
(10) C₁-₆ alkylsulfonyl,
(11) C₃₋₆ cycloalkyl,
(12) C₁₋₆ alkyl-carbonyl,
(13) C₁₋₆ alkyl-carbonyloxy,
(14) carboxy which may be esterified, and
(15) C₁₋₆ alkyl.
   When there are 2 or more substituents, they may be the same or different.

The “C₆₋₁₄ aryl-carbonyl” of the “substituted or unsubstituted C₆₋₁₄ aryl-carbonyl” may have 1 to 5, and preferably 1 to 3 substituents at replaceable positions.

Examples of such substituents include
(1) a halogen atom (e.g., a fluorine atom),
(2) hydroxy,
(3) C₁₋₆ alkyl which may be halogenated,
(4) C₁₋₆ alkoxy which may be substituted with 1 to 3 substituents selected from the group consisting of:

(i) a halogen atom (e.g., a fluorine atom);

(ii) hydroxy;

(iii) C₃₋₆ cycloalkyl (e.g., cyclopropyl); and

(iv) di C₁₋₆ alkylamino (e.g., dimethylamino),
(5) amino,
(6) C₁₋₆ alkylamino,
(7) di C₁₋₆ alkylamino,
(8) C₁₋₆ alkylthio,
(9) C₁-₆ alkylsulfonyl,
(10) C₃₋₆ cycloalkyl,
(11) C₁₋₆ alkyl-carbonyl,
(12) C₁₋₆ alkyl-carbonyloxy, and
(13) carboxy which may be esterified.
   When there are 2 or more substituents, they may be the same or different.

Examples of the “substituted or unsubstituted cyclic group” include a cyclic hydrocarbon group and a heterocyclic group.

Examples of the “cyclic hydrocarbon group” include an alicyclic hydrocarbon group constituted by 3 to 14 carbon atoms and an aromatic hydrocarbon group constituted by 6 to 14 carbon atoms.

Examples of the “alicyclic hydrocarbon group” include C₃₋₆ cycloalkyl (e.g., cyclopropyl, cyclobutyl, cyclopentyl and cyclohexyl), C₃₋₆ cycloalkenyl (e.g., cyclopentenyl and cyclohexenyl), C₅₋₁₄ cycloalkadienyl (e.g., 2,4-cyclopentadienyl and 1,3-cyclohexadienyl), indanyl and adamantyl.

Examples of the “aromatic hydrocarbon group” include C₆₋₁₄ aryl (e.g., phenyl, naphthyl, anthryl and phenanthryl).

Examples of the “heterocyclic group” include the aforementioned aromatic heterocyclic group (e.g., pyridyl, pyridazinyl, oxazolyl, quinolyl, pyrimidinyl and pyrazolyl) and non-aromatic heterocyclic group (e.g., 2,3-dihydrobenzofuranyl).

Preferred examples of the “cyclic group” include C₃₋₆ cycloalkyl (e.g., cyclopropyl, cyclobutyl, cyclopentyl and cyclohexyl), C₆₋₁₄ aryl (e.g., phenyl, naphthyl, anthryl and phenanthryl), and a 4- to 7-membered heterocyclic group (e.g., furyl, thienyl, pyrrolyl, oxazolyl, isoxazolyl, thiazolyl, isothiazolyl, imidazolyl, pyrazolyl, 1,2,3-oxadiazolyl, 1,2,4-oxadiazolyl, 1,3,4-oxadiazolyl, furazanyl, 1,2,3-thiadiazolyl, 1,2,4-thiadiazolyl, 1,3,4-thiadiazolyl, 1,2,3-triazolyl, 1,2,4-triazolyl, tetrazolyl, pyridyl, pyridazinyl, pyrimidinyl, pyrazinyl, triazinyl, azetidinyl, oxetanyl, thiethanyl, pyrrolidinyl, tetrahydrofuryl, thiolanyl, imidazolidinyl, pyrazolidinyl, oxazolidinyl, thiazolidinyl, piperidyl, tetrahydropyranyl, morpholinyl, thiomorpholinyl and piperazinyl).

The “substituted or unsubstituted cyclic group” and “substituted or unsubstituted heterocyclic ring-sulfonyl” may have 1 to 5, and preferably 1 to 3 substituents at replaceable positions.

Examples of such substituents include
(1) a halogen atom,
(2) oxo,
(3) hydroxy,
(4) amino,
(5) nitro,
(6) cyano,
(7) C₁₋₆ alkyl which may be substituted with 1 to 3 substituents selected from the group consisting of:
(i) a halogen atom; and
(ii) a 4- to 7-membered heterocyclic ring (e.g., imidazole),
(8) C₂₋₆ alkenyl,
(9) C₂₋₆ alkynyl,
(10) C₃₋₆ cycloalkyl,
(11) C₆₋₁₄ aryl which may be substituted with 1 to 3 C₁₋₆ alkoxy groups,
(12) C₇₋₁₆ aralkyl,
(13) C₁₋₆ alkoxy which may be substituted with 1 to 3 halogen atoms and C₁₋₆ alkoxy groups, and
(14) C₁₋₆ alkylsulfonyl.
   When there are 2 or more substituents, they may be the same or different.

When R¹ is —OR′ or —SR′, R′ means a hydrogen atom, substituted or unsubstituted C₁₋₆ alkyl, substituted or unsubstituted C₃₋₆ cycloalkyl, or substituted or unsubstituted cyclic group. When R¹ is —SOR″ or —SO₂R″, R″ means substituted or unsubstituted C₁₋₆ alkyl, substituted or unsubstituted C₃₋₆ cycloalkyl, or substituted or unsubstituted cyclic group.

Examples of the “substituted or unsubstituted cyclic group” represented by R′ and “substituted or unsubstituted cyclic group” represented by R″ include those groups listed as the above-described “substituted or unsubstituted cyclic group” exemplified as the “substituent” of the “substituted or unsubstituted C₁₋₆ alkyl”. When there are 2 or more substituents, they may be the same or different.

Preferred examples of the “substituted or unsubstituted cyclic group” represented by R′ and “substituted or unsubstituted cyclic group” represented by R″ include cyclopropyl, cyclobutyl, cyclopentyl, phenyl, and tetrahydropyranyl.

Preferred examples of the above-described Substituent Group A include a group (hereinafter also referred to as “Substituent Group AA”) consisting of, for example,
(1) a halogen atom,
(2) hydroxy,
(3) amino which may be substituted with 1 to 2 substituents selected from the group consisting of:

(i) C₁₋₆ alkyl which may be substituted with cyano; and

(ii) C₁₋₆ alkyl-carbonyl which may be substituted with cyano,
(4) cyano,
(5) C₁₋₆ alkyl which may be substituted with hydroxy,
(6) C₃₋₆ cycloalkyl which may be substituted with 1 to 5 (preferably 1 to 3) substituents selected from the group consisting of:

(a) a halogen atom;

(b) hydroxy;

(c) cyano; and

(d) C₁₋₆ alkyl,
(7) C₁₋₆ alkoxy which may be substituted with 1 to 5 (preferably 1 to 3) substituents selected from the group consisting of
(a) C₁₋₆ alkoxy which may be substituted with 1 to 3 substituents selected from the group consisting of:

(i) a halogen atom (e.g., a fluorine atom);

(ii) hydroxy;

(iii) a C₃₋₆ cycloalkyl group (e.g., cyclopropyl); and

(iv) di C₁₋₆ alkylamino group (e.g., dimethylamino), and
(b) C₁₋₆ alkyl,
(8) C₆₋₁₄ aryloxy which may be substituted with a halogen atom,
(9) 5- or 6-membered heterocyclic ring-oxy (e.g., tetrahydropyranyloxy),
(10) C₁₋₆ alkylamino which may be substituted with cyano,
(11) di C₁₋₆ alkylamino,
(12) C₁₋₆ alkyl-carbonylamino which may be substituted with cyano,
(13) C₁₋₆ alkylsulfonyl which may be substituted with 1 to 5 (preferably 1 to 3) substituents selected from the group consisting of:
(a) C₃₋₆ cycloalkyl; and
(b) C₁₋₆ alkyl,
(14) 5- or 6-membered heterocyclic ring-sulfonyl (e.g., morpholinylsulfonyl),
(15) carboxy,
(16) C₁₋₆ alkoxy-carbonyl,
(17) 5- or 6-membered heterocyclic ring-carbonyl which may be substituted with 1 to 3 substituents selected from the group consisting of:
(i) hydroxy;
(ii) oxo; and
(iii) C₁₋₆ alkyl (e.g., morpholinylcarbonyl, pyrrolidinylcarbonyl, piperidinylcarbonyl and thiomorpholinylcarbonyl),
(18) C₃₋₆ cycloalkyl-carbamoyl,
(19) C₁₋₆ alkyl-carbamoyl which may be substituted with 1 to 5 (preferably 1 to 3) substituents selected from the group consisting of
(a) a halogen atom,
(b) hydroxy,
(c) C₁₋₆ alkoxy which may be substituted with 1 to 3 substituents selected from the group consisting of:

(i) a halogen atom (e.g., a fluorine atom);

(ii) hydroxy;

(iii) a C₃₋₆ cycloalkyl group (e.g., cyclopropyl); and

(iv) di C₁₋₆ alkylamino group (e.g., dimethylamino),
(d) cyano,
(e) C₁₋₆ alkylsulfonyl,
(f) C₃₋₆ cycloalkyl,
(g) C₁₋₆ alkyl-carbonyl,
(h) C₁₋₆ alkyl-carbonyloxy and
(i) C₁₋₆ alkoxy-carboxy,
(20) di C₁₋₆ alkyl-carbamoyl which may be substituted with cyano,
(21) C₁₋₃ alkylidenecarbamoyl,
(22) C₁₋₆ alkylsulfonyl-carbamoyl,
(23) sulfamoyl which may be substituted with C₃₋₆ cycloalkyl-carbonyl,
(24) C₁₋₆ alkylsulfamoyl which may be substituted with 1 to 5 (preferably 1 to 3) substituents selected from the group consisting of
(a) a halogen atom,
(b) hydroxy and
(b) cyano,
(25) C₃₋₆ cycloalkylsulfamoyl,
(26) a 5- or 6-membered cyclic group which may be substituted with 1 to 5 (preferably 1 to 3) substituents selected from the group consisting of
(a) a halogen atom,
(b) hydroxy and
(c) C₁₋₆ alkyl which may be substituted with imidazole (e.g., phenyl, cyclohexyl, pyridyl, tetrazolyl, imidazolyl, tetrahydropyranyl, morpholinyl, piperidinyl and oxetanyl), and
(27) silyloxy which may be substituted with 1 to 3 C₁₋₆ alkyl groups.
   When there are 2 or more substituents, they may be the same or different.

The “5- or 6-membered heterocyclic ring” of the “5- or 6-membered heterocyclic ring-oxy”, “5- or 6-membered heterocyclic ring-sulfonyl” and “5- or 6-membered heterocyclic ring-carbonyl” indicates a “5- or 6-membered heterocyclic group”. Examples thereof include furyl, thienyl, pyrrolyl, oxazolyl, isoxazolyl, thiazolyl, isothiazolyl, imidazolyl, pyrazolyl, 1,2,3-oxadiazolyl, 1,2,4-oxadiazolyl, 1,3,4-oxadiazolyl, furazanyl, 1,2,3-thiadiazolyl, 1,2,4-thiadiazolyl, 1,3,4-thiadiazolyl, 1,2,3-triazolyl, 1,2,4-triazolyl, tetrazolyl, pyridyl, pyridazinyl, pyrimidinyl, pyrazinyl, triazinyl, azetidinyl, oxetanyl, thiethanyl, pyrrolidinyl, tetrahydrofuryl, thiolanyl, imidazolidinyl, pyrazolidinyl, oxazolidinyl, thiazolidinyl, piperidinyl, tetrahydropyranyl, morpholinyl, thiomorpholinyl and piperazinyl.

R¹ is preferably —OR′ or —SR′ (wherein R′ is C₁₋₆ alkyl, C₃₋₆ cycloalkyl or C₆₋₁₄ aryl which may be substituted with 1 to 5 (preferably 1 to 3) substituents selected from the group consisting of (a) a halogen atom, (b) C₁₋₆ alkoxy which may be substituted with 1 to 3 C₁₋₆ alkoxy groups, (c) C₃₋₆ cycloalkyl and (d) C₁₋₆ alkylsulfonyl).

When R¹ is hydroxy (—OH) or thioxy (—SH), tautomers are also included in the compound represented by formula (I) or a salt thereof. Specific examples of such tautomers include:

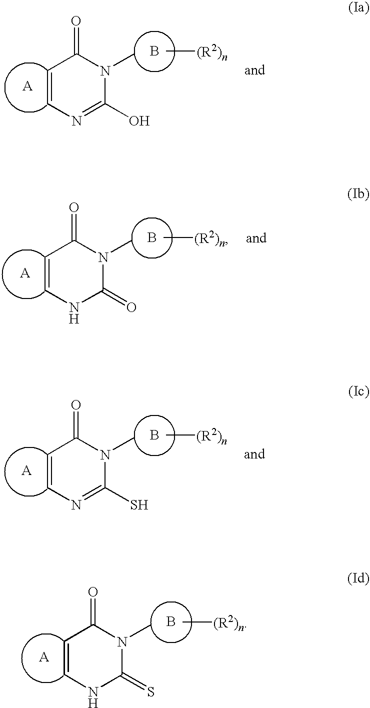

R² means a hydrogen atom, a halogen atom, substituted or unsubstituted C₁₋₆ alkyl, or substituted or unsubstituted C₁₋₆ alkoxy. In this regard, n means an integer from 1 to 5.

The “substituted or unsubstituted ₁₋₆ alkyl” and “substituted or unsubstituted C₁₋₆ alkoxy” represented by R² may be substituted with 1 to 9, and preferably 1 to 5 substituents selected from the above-described Substituent Group A at replaceable positions.

Preferred examples of such substituents include
(1) a halogen atom,
(2) hydroxy, and
(3) C₃₋₆ cycloalkyl which may be substituted with 1 to 3 substituents selected from the group consisting of:

(i) a halogen atom; and

(ii) C₁₋₆ alkyl.

When there are 2 or more substituents, they may be the same or different.

R² is preferably a hydrogen atom, a halogen atom, or substituted or unsubstituted C₁₋₆ alkoxy (preferably, C₁₋₆ alkoxy may be substituted with 1 to 9, and preferably 1 to 5 substituents selected from the group consisting of a halogen atom and C₃₋₆ cycloalkyl), and n is preferably 1 or 2. n is more preferably 1.

In formula (I), the condensed ring including Ring A is represented by the following formula (a) or (b).

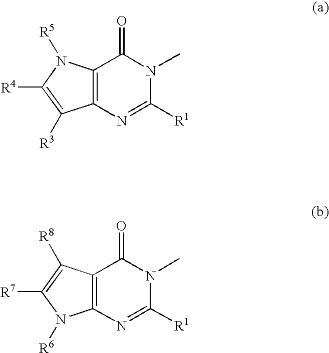

R³ means a hydrogen atom, substituted or unsubstituted C₁₋₆ alkyl, or substituted or unsubstituted C₃₋₈ cycloalkyl.

The “substituted or unsubstituted C₁₋₆ alkyl” and “substituted or unsubstituted C₃₋₈ cycloalkyl” represented by R³ may be substituted with 1 to 5, and preferably 1 to 3 substituents selected from the above-described Substituent Group AA at replaceable positions. When there are 2 or more substituents, they may be the same or different.

R³ is preferably a hydrogen atom, C₁₋₆ alkyl or C₃₋₈ cycloalkyl.

R³ is particularly preferably a hydrogen atom.

R⁴ means a hydrogen atom, a halogen atom, hydroxy, substituted or unsubstituted C₁₋₆ alkyl, or substituted or unsubstituted C₁₋₆ alkoxy.

The “substituted or unsubstituted C₁₋₆ alkyl” and “substituted or unsubstituted C₁₋₆ alkoxy” represented by R⁴ may be substituted with 1 to 5, and preferably 1 to 3 substituents selected from the above-described Substituent Group AA at replaceable positions. When there are 2 or more substituents, they may be the same or different.

When R⁴ is hydroxy, tautomers are also included in the compound represented by formula (I) or a salt thereof. Specific examples thereof include:

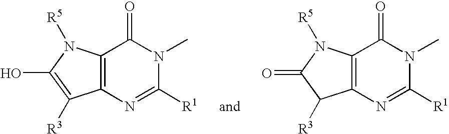

R⁴ is preferably a hydrogen atom, hydroxy, C₁₋₆ alkyl or C₁₋₆ alkoxy.

R⁴ is particularly preferably a hydrogen atom.

R⁵ means a hydrogen atom, or substituted or unsubstituted C₁₋₆ alkyl.

R⁵ is preferably a hydrogen atom or C₁₋₆ alkyl.

R⁵ is more preferably a hydrogen atom or methyl.

R⁵ is particularly preferably a hydrogen atom.

R⁶ means a hydrogen atom, substituted or unsubstituted C₁₋₆ alkyl, or substituted or unsubstituted C₃₋₈ cycloalkyl.

The “substituted or unsubstituted C₁₋₆ alkyl” and “substituted or unsubstituted C₃₋₈ cycloalkyl” represented by R⁶ may be substituted with 1 to 5, and preferably 1 to 3 substituents selected from the above-described Substituent Group AA at replaceable positions.

Preferred examples of such substituents include
(1) a halogen atom,
(2) hydroxy,
(3) C₁₋₆ alkoxy,
(4) substituted or unsubstituted C₁₋₆ alkyl-carbamoyl, and
(5) C₃₋₆ cycloalkyl which may be substituted with C₁₋₆ alkyl.
   When there are 2 or more substituents, they may be the same or different.

The above-described “substituted or unsubstituted C₁₋₆ alkyl-carbamoyl” may have 1 to 5, and preferably 1 to 3 substituents at replaceable positions. Examples of such substituents include
(1) a halogen atom,
(2) hydroxy,
(3) C₁₋₆ alkoxy which may be substituted with 1 to 3 substituents selected from the group consisting of:

(i) a halogen atom (e.g., a fluorine atom);

(ii) hydroxy;

(iii) C₃₋₆ cycloalkyl (e.g., cyclopropyl); and

(iv) di C₁₋₆ alkylamino (e.g., dimethylamino),
(4) amino,
(5) cyano,
(6) C₁₋₆ alkylamino,
(7) di C₁₋₆ alkylamino,
(8) C₁₋₆ alkylthio,
(9) C₁₋₆ alkylsulfonyl,
(10) C₃₋₆ cycloalkyl,
(11) C₁₋₆ alkyl-carbonyl,
(12) C₁₋₆ alkyl-carbonyloxy,
(13) carboxy which may be esterified, and
(14) silyloxy which may be substituted with 1 to 3 C₁₋₆ alkyl groups.
   When there are 2 or more substituents, they may be the same or different.

R⁶ is preferably a hydrogen atom, or substituted or unsubstituted C₁₋₆ alkyl.

R⁶ is more preferably a hydrogen atom, or C₁₋₆ alkyl which may be substituted with C₁₋₆ alkoxy.

R⁷ means a hydrogen atom, a halogen atom, substituted or unsubstituted hydroxy, C₂₋₆ alkyl, substituted C₁₋₆ alkyl, or substituted or unsubstituted C₁₋₆ alkoxy.

The “substituted C₁₋₆ alkyl” represented by R⁷ is substituted with 1 to 5, and preferably 1 to 3 substituents selected from the above-described Substituent Group A at replaceable positions. When there are 2 or more substituents, they may be the same or different.

The “substituted or unsubstituted hydroxy” and “substituted or unsubstituted C₁₋₆ alkoxy” represented by R⁷ may be substituted with 1 to 5, and preferably 1 to 3 substituents selected from the above-described Substituent Group A at replaceable positions. Preferred examples of such substituents include C₁₋₆ alkyl-carbonyl. When there are 2 or more substituents, they may be the same or different.

When R⁷ is hydroxy, tautomers are also included in the compound represented by formula (I) or a salt thereof. Specific examples of such tautomers include:

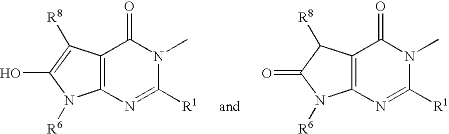

R⁷ is preferably a hydrogen atom, a halogen atom or hydroxy.

R⁸ is a hydrogen atom or a halogen atom.

R⁸ is preferably a hydrogen atom.

Ring B means a 5- or 6-membered ring. In this regard, examples of the “5- or 6-membered ring” include benzene, C₅₋₆ cycloalkane, C₅₋₆ cycloalkene, C₅₋₆ cycloalkadiene, a 5- or 6-membered aromatic heterocyclic ring, and a 5- or 6-membered non-aromatic heterocyclic ring.

Examples of the C₅₋₆ cycloalkane include cyclopentane and cyclohexane.

Examples of the C₅₋₆ cycloalkene include 1-cyclopentene and 1-cyclohexene.

Examples of the C₅₋₆ cycloalkadiene include 1,3-cyclopentadiene, 1,3-cyclohexadiene and 1,4-cyclohexadiene.

Examples of the 5- or 6-membered aromatic heterocyclic ring include pyrrole, imidazole, pyrazole, pyrazine, pyridine, pyrimidine, furan, oxazole, isoxazole, thiophene, thiazole, and isothiazole.

Examples of the 5- or 6-membered non-aromatic heterocyclic ring include pyrrolidine, imidazolidine, piperidine, piperazine, and tetrahydrofuran.

In this regard, when R⁴ is a hydrogen atom, a halogen atom, substituted or unsubstituted C₁₋₆ alkyl or substituted or unsubstituted C₁₋₆ alkoxy, and when R⁷ is a hydrogen atom, a halogen atom, substituted hydroxy, C₂₋₆ alkyl, substituted C₁₋₆ alkyl or substituted or unsubstituted C₁₋₆ alkoxy, Ring B means the following formula (c):

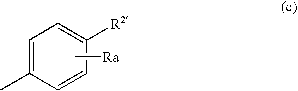

In the formula, R^{2′} is substituted or unsubstituted C₁₋₆ alkyl, or substituted or unsubstituted C₁₋₆ alkoxy.

The “substituted or unsubstituted C₁₋₆ alkyl” and “substituted or unsubstituted C₁₋₆ alkoxy” represented by R^{2′} may be substituted with 1 to 9, and preferably 1 to 5 substituents selected from the above-described Substituent Group A at replaceable positions.

Preferred examples of such substituents include
(1) a halogen atom,
(2) hydroxy, and
(3) C₃₋₆ cycloalkyl which may be substituted with 1 to 3 substituents selected from the group consisting of:

(i) a halogen atom; and

(ii) C₁₋₆ alkyl.

When there are 2 or more substituents, they may be the same or different.

R^{2′} is preferably substituted or unsubstituted C₁₋₆ alkoxy (preferably, C₁₋₆ alkoxy may be substituted with 1 to 9, and preferably 1 to 5 substituents selected from the group consisting of a halogen atom and C₃₋₆ cycloalkyl).

Ra is a hydrogen atom, a halogen atom, substituted or unsubstituted C₁₋₆ alkyl, or substituted or unsubstituted C₁₋₆ alkoxy.

The “substituted or unsubstituted C₁₋₆ alkyl” and “substituted or unsubstituted C₁₋₆ alkoxy” represented by Ra may be substituted with 1 to 5, and preferably 1 to 3 substituents selected from the above-described Substituent Group A at replaceable positions. When there are 2 or more substituents, they may be the same or different.

Ra is preferably a hydrogen atom or a halogen atom.

Ring B is preferably a ring represented by the following formula:

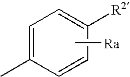

(wherein R^{2′} is C₁₋₆ alkoxy which may be substituted with a substituent selected from the group consisting of a halogen atom and C₃₋₆ cycloalkyl, and Ra is a hydrogen atom or a halogen atom).

Examples of preferred embodiments of the compound (I) are as described below.

### [Compound A 1]

Compound (I) represented by formula (I), wherein:
R¹ is —OR′, —SR′, —SOR″ or —SO₂R″ (wherein R′ is a hydrogen atom, substituted or unsubstituted C₁₋₆ alkyl, substituted or unsubstituted C₃₋₆ cycloalkyl, or substituted or unsubstituted cyclic group, and R″ is substituted or unsubstituted C₁₋₆ alkyl, substituted or unsubstituted C₃₋₆ cycloalkyl, or a substituted or unsubstituted cyclic group);
R² is a hydrogen atom, a halogen atom, or substituted or unsubstituted C₁₋₆ alkoxy (preferably, C₁₋₆ alkoxy may be substituted with 1 to 9, and preferably 1 to 5 substituents selected from the group consisting of a halogen atom and C₃₋₆ cycloalkyl);
n is 1 or 2;
the condensed ring including Ring A is represented by the following formula (a):

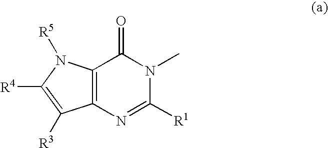

wherein:

R³ is a hydrogen atom, C₁₋₆ alkyl or C₃₋₈ cycloalkyl,

R⁴ is a hydrogen atom, hydroxy, C₁₋₆ alkyl, or C₁₋₆ alkoxy, and

R⁵ is a hydrogen atom or C₁₋₆ alkyl; and
Ring B is benzene.

### [Compound A2]

Compound (I) represented by formula (I), wherein:
R¹ is —OR′, —SR′, —SOR″ or —SO₂R″ (wherein R′ is a hydrogen atom, substituted or unsubstituted C₁₋₆ alkyl, substituted or unsubstituted C₃₋₆ cycloalkyl, or substituted or unsubstituted cyclic group, and R″ is substituted or unsubstituted C₁₋₆ alkyl, substituted or unsubstituted C₃₋₆ cycloalkyl, or substituted or unsubstituted cyclic group);
the condensed ring including Ring A is represented by the following formula (a):

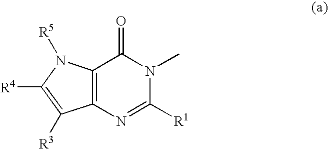

wherein:

R³ is a hydrogen atom, C₁₋₆ alkyl or C₃₋₈ cycloalkyl,

R⁴ is a hydrogen atom, hydroxy, C₁₋₆ alkyl or C₁₋₆ alkoxy, and

R⁵ is a hydrogen atom or C₁₋₆ alkyl; and
Ring B is a ring represented by the following formula (c):

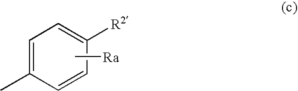

(wherein

R^{2′} is substituted or unsubstituted C₁₋₆ alkyl, or substituted or unsubstituted C₁₋₆ alkoxy, and

Ra is a hydrogen atom, a halogen atom, substituted or unsubstituted C₁₋₆ alkyl, or substituted or unsubstituted C₁₋₆ alkoxy).

### [Compound A3]

Compound (I) represented by formula (I), wherein:
R¹ is —OR′ or —SR′ (wherein R′ is C₁₋₆ alkyl, C₃₋₆ cycloalkyl or C₆₋₁₄ aryl, which may be substituted with 1 to 5, and preferably 1 to 3 substituents selected from the group consisting of: (a) a halogen atom; (b) C₁₋₆ alkoxy which may be substituted with 1 to 3 C₁₋₆ alkoxy groups; (c) C₃₋₆ cycloalkyl; and (d) C₁₋₆ alkylsulfonyl);
the condensed ring including Ring A is represented by the following formula (a):

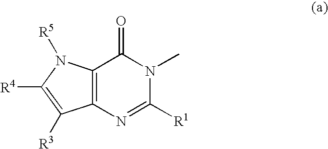

wherein:

R³ is a hydrogen atom, C₁₋₆ alkyl or C₃₋₈ cycloalkyl,

R⁴ is a hydrogen atom, hydroxy, C₁₋₆ alkyl or C₁₋₆ alkoxy, and

R⁵ is a hydrogen atom or C₁₋₆ alkyl; and
Ring B is a ring represented by the following formula (c):

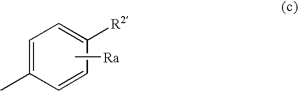

(wherein

R^{2′} is substituted or unsubstituted C₁₋₆ alkyl, or substituted or unsubstituted C₁₋₆ alkoxy, and

Ra is a hydrogen atom, a halogen atom, substituted or unsubstituted C₁₋₆ alkyl, or substituted or unsubstituted C₁₋₆ alkoxy).

### [Compound A4]

Compound (I) represented by the following formula:

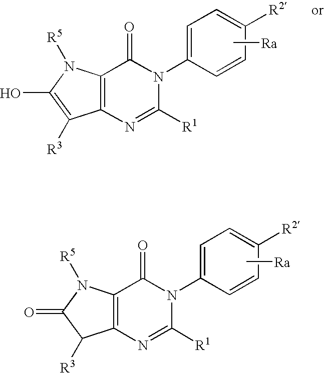

(wherein:
R¹ is —OR′ or —SR′ (wherein R′ is C₁₋₆ alkyl, C₃₋₆ cycloalkyl or C₆₋₁₄ aryl, which may be substituted with 1 to 5, and preferably 1 to 3 substituents selected from the group consisting of: (a) a halogen atom; (b) C₁₋₆ alkoxy which may be substituted with 1 to 3 C₁₋₆ alkoxy groups; (c) C₃₋₆ cycloalkyl; and (d) C₁₋₆ alkylsulfonyl);
R³ is a hydrogen atom, C₁₋₆ alkyl or C₃₋₈ cycloalkyl;
R⁵ is a hydrogen atom or C₁₋₆ alkyl;
R^{2′} is C₁₋₆ alkoxy which may be substituted with 1 to 9 substituents selected from the group consisting of a halogen atom and C₃₋₆ cycloalkyl; and
Ra is a hydrogen atom or a halogen atom).

### [Compound B1]

Compound (I) represented by formula (I), wherein:
R¹ is —OR′, —SR′, —SOR″ or —SO₂R″ (wherein R′ is a hydrogen atom, substituted or unsubstituted C₁₋₆ alkyl, substituted or unsubstituted C₃₋₆ cycloalkyl, or substituted or unsubstituted cyclic group, and R″ is substituted or unsubstituted C₁₋₆ alkyl, substituted or unsubstituted C₃₋₆ cycloalkyl, or substituted or unsubstituted cyclic group);
R² is a hydrogen atom, a halogen atom, or substituted or unsubstituted C₁₋₆ alkoxy (preferably, C₁₋₆ alkoxy may be substituted with 1 to 9, and preferably 1 to 5 substituents selected from the group consisting of a halogen atom and C₃₋₆ cycloalkyl);
n is 1 or 2;
the condensed ring including Ring A is represented by the following formula (b):

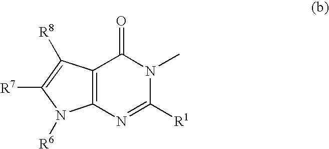

wherein

R⁶ is a hydrogen atom or substituted or unsubstituted C₁₋₆ alkyl,

R⁷ is a hydrogen atom, a halogen atom or hydroxy, and

R⁸ is a hydrogen atom or a halogen atom; and
Ring B is benzene.

### [Compound B2]

Compound (I) represented by formula (I), wherein:
R¹ is —OR′, —SR′, —SOR″ or —SO₂R″ (wherein R′ is a hydrogen atom, substituted or unsubstituted C₁₋₆ alkyl, substituted or unsubstituted C₃₋₆ cycloalkyl, or substituted or unsubstituted cyclic group, and R″ is substituted or unsubstituted C₁₋₆ alkyl, substituted or unsubstituted C₃₋₆ cycloalkyl, or substituted or unsubstituted cyclic group);
the condensed ring including Ring A is represented by the following formula (b):

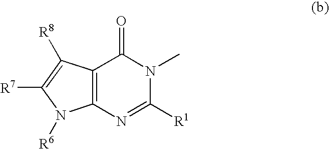

wherein

R⁶ is a hydrogen atom, or substituted or unsubstituted C₁₋₆ alkyl,

R⁷ is a hydrogen atom, a halogen atom or hydroxy, and

R⁸ is a hydrogen atom or a halogen atom; and
Ring B is a ring represented by the following formula (c):

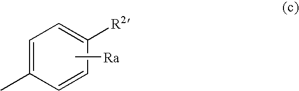

(wherein

R^{2′} is substituted or unsubstituted C₁₋₆ alkyl, or substituted or unsubstituted C₁₋₆ alkoxy, and

Ra is a hydrogen atom, a halogen atom, substituted or unsubstituted C₁₋₆ alkyl, or substituted or unsubstituted C₁₋₆ alkoxy).

### [Compound B3]

Compound (I) represented by formula (I), wherein:
R¹ is —OR′ or —SR′ (wherein R′ is C₁₋₆ alkyl, C₃₋₆ cycloalkyl or C₆₋₁₄ aryl, which may be substituted with 1 to 5, and preferably 1 to 3 substituents selected from the group consisting of: (a) a halogen atom; (b) C₁₋₆ alkoxy which may be substituted with 1 to 3 C₁₋₆ alkoxy groups; (c) C₃₋₆ cycloalkyl; and (d) C₁₋₆ alkylsulfonyl);
the condensed ring including Ring A is represented by the following formula (b):

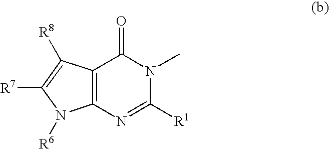

wherein

R⁶ is a hydrogen atom, or C₁₋₆ alkyl which may be substituted with C₁₋₆ alkoxy,

R⁷ is a hydrogen atom, a halogen atom or hydroxy, and

R⁸ is a hydrogen atom or a halogen atom; and
Ring B is a ring represented by the following formula (c):

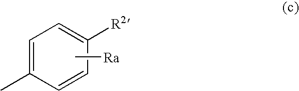

(wherein

R^{2′} is substituted or unsubstituted C₁₋₆ alkyl, or substituted or unsubstituted C₁₋₆ alkoxy, and

Ra is a hydrogen atom, a halogen atom, substituted or unsubstituted C₁₋₆ alkyl, or substituted or unsubstituted C₁₋₆ alkoxy).

### [Compound B4]

Compound (I) represented by the following formula:

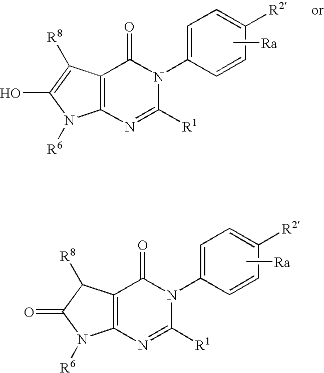

(wherein:
R¹ is —OR′ or —SR′ (wherein R′ is C₁₋₆ alkyl, C₃₋₆ cycloalkyl or C₆₋₁₄ aryl, which may be substituted with 1 to 5 substituents selected from the group consisting of: (a) a halogen atom; (b) C₁₋₆ alkoxy which may be substituted with 1 to 3 C₁₋₆ alkoxy groups; (c) C₃₋₆ cycloalkyl; and (d) C₁₋₆ alkylsulfonyl);
R⁶ is a hydrogen atom, or C₁₋₆ alkyl which may be substituted with C₁₋₆ alkoxy;
R⁸ is a hydrogen atom or a halogen atom;
R^{2′} is C₁₋₆ alkoxy which may be substituted with 1 to 9 substituents selected from the group consisting of a halogen atom and C₃₋₆ cycloalkyl; and
Ra is a hydrogen atom or a halogen atom).

### [Compound C]

2-(2,2,2-trifluoroethoxy)-3-[4-(2,2,2-trifluoroethoxy)phenyl]-5,7-dihydro-3H-pyrrolo[2,3-d]pyrimidine-4,6-dione or a salt thereof,

2-(2,2,3,3,3-pentafluoropropoxy)-3-[4-(2,2,2-trifluoroethoxy)phenyl]-5,7-dihydro-3H-pyrrolo[2,3-d]pyrimidine-4,6-dione or a salt thereof,

2-[(cyclopropylmethyl)sulfanyl]-3-[4-(2,2,2-trifluoroethoxy)phenyl]-5,7-dihydro-3H-pyrrolo[2,3-d]pyrimidine-4,6-dione or a salt thereof,

2-(2,2,2-trifluoroethoxy)-3-[4-(2,2,2-trifluoroethoxy)phenyl]-3,7-dihydro-4H-pyrrolo[2,3-d]pyrimidine-4-one or a salt thereof,

2-(2,2,3,3,3-pentafluoropropoxy)-3-[4-(2,2,2-trifluoroethoxy)phenyl]-3,7-dihydro-4H-pyrrolo[2,3-d]pyrimidine-4-one or a salt thereof, or

2-[(cyclopropylmethyl)sulfanyl]-3-[4-(2,2,2-trifluoroethoxy)phenyl]-3,7-dihydro-4H-pyrrolo[2,3-d]pyrimidine-4-one or a salt thereof.

Examples of salts of the compound represented by Formula (I) include pharmacologically acceptable salts such as acid addition salts of acid such as trifluoroacetic acid, acetic acid, lactic acid, succinic acid, maleic acid, tartaric acid, citric acid, gluconic acid, ascorbic acid, benzoic acid, methanesulfonic acid, p-toluenesulfonic acid, cinnamic acid, fumaric acid, phosphonic acid, hydrochloric acid, nitric acid, hydrobromic acid, hydriodic acid, sulfamic acid, sulfuric acid or the like; for example, salts of metal such as sodium, potassium, magnesium, calcium or the like; for example, salts with organic base such as trimethylamine, triethylamine, pyridine, picoline, N-methylpyrrolidine, N-methylpiperidine, N-methylmorpholine or the like.

A prodrug of Compound (I) refers to a compound that is converted into Compound (I) upon reaction with an enzyme, gastric acid or the like under in vivo physiological conditions, namely, a compound that is converted into Compound (I) upon enzymatic oxidation, reduction, hydrolysis or the like, or a compound that is converted into Compound (I) upon hydrolysis or the like by gastric acid or the like. Examples of prodrugs of Compound (I) include compounds having the amino group of Compound (I) acylated, alkylated or phosphorylated (e.g., compounds having the amino group of Compound (I) eicosanoylated, alanylated, pentylaminocarbonylated, (5-methyl-2-oxo-1,3-dioxole-4-yl)methoxycarbonylated, tetrahydrofuranylated, pyrrolidylmethylated, pivaloyloxymethylated, tert-butylated or the like), compounds having the hydroxyl group of Compound (I) acylated, alkylated, phosphorylated or borated (e.g., compounds having the hydroxyl group of Compound (I) acetylated, palmitoylated, propanoylated, pivaloylated, succinylated, fumarylated, alanylated, dimethylaminomethylcarbonylated or the like), and compounds having the carboxyl group of Compound (I) esterified or amidated (e.g., compounds having the carboxyl group of Compound (I) ethyl-esterified, phenyl-esterified, carboxymethyl-esterified, dimethylaminomethyl-esterified, pivaloyloxymethyl-esterified, ethoxycarbonyloxyethyl-esterified, phthalidyl-esterified, (5-methyl-2-oxo-1,3-dioxolen-4-yl)methyl-esterified, cyclohexyloxycarbonylethyl-esterified, methylamidated, or the like). These compounds may be produced from Compound (I) by a method known per se.

In addition, a prodrug of Compound (I) may be one that is converted into Compound (I) under physiological conditions described in Iyakuhin No Kaihatsu (Development of Medicine), Vol. 7, Molecular Design, pp. 163-198 (Hirokawa Shoten, 1990).

When Compound (I) has isomers such as optical isomers, stereoisomers, positional isomers or rotational isomers, either one of the isomers or a mixture of the isomers are comprised in Compound (I). For example, if optical isomers of Compound (I) exist, an optical isomer separated from the racemic form is also comprised in Compound (I). Each of these isomers may be obtained alone by a synthetic technique or a separation technique (concentration, solvent extraction, column chromatography, recrystallization, etc.) known per se.

Compound (I) may be either crystalline or amorphous. When Compound (I) is crystalline, either single or a mixture of crystalline forms may be comprised in Compound (I). A crystal may be produced by crystallization by applying a crystallization technique known per se.

Compound (I) may also be a pharmaceutically acceptable cocrystal or cocrystallized salt. Here, each of cocrystals or cocrystallized salts has different physical properties (e.g., structure, melting point, melting heat, hygroscopicity, solubility, stability, etc.), and refers to a crystalline substance that is comprised of two or more types of distinctive solids at room temperature. A cocrystal or cocrystallized salt may be produced according to a cocrystallization technique known per se.

Compound (I) may be either a solvate (e.g., hydrate, etc.) or a non-solvate, and both are comprised in Compound (I).

Compound (I) may be labeled with an isotope (e.g., ³H, ¹⁴C, ³⁵S, ¹²⁵I, etc.) or the like.

Furthermore, deuterium-exchanged compounds where ¹H is converted into ²H (D) are also comprised in Compound (I).

Since Compound (I) or a prodrug thereof (hereinafter, abbreviated as a “compound of the invention”) has a potent delta-5-desaturase inhibitory effect, it is useful as a prophylactic or therapeutic drug for a disease triggered (or a disease whose onset is induced) by involvement of eicosanoid that is produced via delta-5-desaturase in mammals (e.g., human, monkey, cat, swine, horse, bovine, mouse, rat, guinea pig, dog, rabbit or the like).

The compound is useful for preventing or treating, for example, such diseases as cardiac diseases (cardiac hypertrophy, acute heart failure and chronic heart failure including congestive heart failure, cardiomyopathy, angina, myocarditis, arrhythmia, tachycardia, myocardial infarction, etc.), myocardial ischemia, venous insufficiency, post-myocardial infarction transition to heart failure, hypertension, cor pulmonale, arteriosclerosis including atherosclerosis (aneurysm, coronary arterial sclerosis, cerebral arterial sclerosis, peripheral arterial sclerosis, etc.), intervention (percutaneous coronary angioplasty, stent placement, coronary angioscopy, intravascular ultrasound, coronary thrombolytic therapy, etc.)—and heart transplantation-related vascular thickening/occlusion/organ damages, vascular reocclusion/restenosis after bypass surgery, respiratory diseases (cold syndrome, pneumonia, asthma, pulmonary hypertension, pulmonary thrombus/pulmonary embolism, etc.), bone disorders (nonmetabolic bone disorders such as bone fracture, refracture, bone malformation/spondylosis deformans, osteosarcoma, myeloma, dysostosis and scoliosis, bone defect, osteoporosis, osteomalacia, rickets, osteitis fibrosis, renal osteodystrophy, Paget's disease of bone, myelitis with rigidity, chronic rheumatoid arthritis, gonarthrosis and articular tissue destruction in similar disorders thereof, etc.), inflammatory diseases (retinopathy, nephropathy, nerve damage, arthritis such as chronic rheumatoid arthritis, osteoarthritis, rheumatoid myelitis and periostitis, inflammation after surgery/trauma, reduction of swelling, pharyngitis, cystitis, atopic dermatitis, inflammatory enteric diseases such as Crohn's disease and ulcerative colitis, meningitis, inflammatory eye diseases, inflammatory pulmonary diseases such as pneumonia, silicosis, pulmonary sarcoidosis and pulmonary tuberculosis, etc.), allergic diseases (allergic rhinitis, conjunctivitis, gastrointestinal allergy, pollen allergy, anaphylaxis, etc.), drug dependence, neurodegenerative diseases (Alzheimer's disease, Parkinson's disease, amyotrophic lateral sclerosis, AIDS encephalopathy, etc.), central nervous system damage (disorders such as cerebral hemorrhage and cerebral infarction and aftereffects and complications thereof, head injury, spinal damage, cerebral edema, etc.), dementia, disturbed memory, disturbed consciousness, amnesia, anxiety symptoms, nervous symptoms, unpleasant condition, mental disorders (depression, epilepsy, alcohol dependency, etc.), ischemic peripheral circulatory disorder, deep-vein thrombosis, occlusive peripheral circulatory disorder, arteriosclerosis obliterans (ASO), occlusive thromboangiitis, diabetes (type 1 diabetes, type 2 diabetes, type 1.5 diabetes (LADA (Latent Autoimmune Diabetes in Adults)), pregnancy diabetes, diabetes with impaired insulin secretion, obese diabetes, impaired glucose tolerance (IGT), IFG (Impaired Fasting Glucose), IFG (Impaired Fasting Glycaemia), etc.), diabetic complications (nerve damage, nephropathy, retinopathy, cataract, macroangiopathy, osteopenia, diabetic hyperosmolar diabetic coma, infectious diseases (respiratory infection; urinary infection, digestive tract infection, skin and soft tissue infection, lower limb infection, etc.), diabetic gangrene, xerostomia, deterioration in hearing, cerebrovascular damage, peripheral circulatory disorder, etc.), urinary incontinence, metabolic/nutritional disorders (obesity (e.g., malignant mastocytosis, exogenous obesity, hyperinsulinar obesity, hyperplasmic obesity, hypophyseal adiposity, hypoplasmic obesity, hypothyroid obesity, hypothalamic obesity, symptomatic obesity, infantile obesity, upper body obesity, alimentary obesity, hypogonadal obesity, systemic mastocytosis, simple obesity, central obesity, etc.), hyperphagia, hyperlipidemia, hypercholesterolemia, impaired glucose tolerance, etc.), insulin resistant syndrome, syndrome X, vesceral obesity syndrome, male or female sexual dysfunction, cerebrovascular damage (asymptomatic cerebrovascular damage, transient cerebral ischemia attack, stroke, cerebrovascular dementia, hypertensive encephalopathy, cerebral infarction, etc.), cerebral edema, cerebral circulatory disturbance, recurrence and aftereffects of cerebrovascular damages (neurological symptoms, mental symptoms, subjective symptoms, impairment of activities of daily living, etc.), kidney diseases (nephritis, glomerulonephritis, glomerulosclerosis, renal failure, thrombotic microangiopathy, diabetic nephropathy, nephrotic syndrome, hypertensive nephrosclerosis, complications of dialysis, organ damage including nephropathy by irradiation, etc.), ocular disorders (glaucoma, ocular hypertension, etc.), thrombosis, multiple organ failure, endothelial dysfunction, other circulatory diseases (ischemic cerebral circulatory disturbance, Raynaud's disease, Buerger's disease, etc.), chronic occlusive pulmonary diseases, interstitial pneumonia, carinii pneumonia, connective tissue disorders (e.g., systemic erythematosus, scleroderma, polyarteritis, etc.), liver disorders (hepatitis and cirrhosis including chronic types, etc.), digestive disorders (gastritis, gastric ulcer, gastric cancer, disorder after gastric surgery, poor digestion, esophageal ulcer, pancreatitis, colon polyp, cholelithiasis, hemorrhoidal problem, esophageal and gastric variceal rupture, etc.), hematological/hematopoietic disorders (erythrocytosis, vascular purpura, autoimmune hemolytic anemia, disseminated intravascular coagulation syndrome, multiple myelosis, etc.), solid tumor, tumors (malignant melanoma, malignant lymphoma, digestive organs (e.g., stomach, intestine, etc.) cancers, etc.), cancers and cachexia associated therewith, cancer metastases, endocrine disorders (Addison's disease, Cushing's syndrome, pheochromocytoma, primary aldosteronism, etc.), urological/male genital diseases (cystitis, prostatic enlargement, prostate cancer, sexually transmitted diseases, etc.), gynecological disorders (menopausal disorders, pregnancy toxemia, endometriosis, uterine fibroid, ovarian diseases, mammary gland diseases, sexually transmitted diseases, etc.), infectious diseases (viral infectious diseases of, for example, cytomegalovirus, influenza virus and herpesvirus, rickettsial infectious diseases, bacterial infectious diseases, etc.), toxemia (septicemia, septic shock, endotoxic shock, gram-negative septicemia, toxin shock syndrome, etc.), cutaneous diseases (keloid, hemangioma, psoriasis, etc.). In particular, the compound is preferably used for preventing or treating atherosclerosis, diabetes or obesity. Herein, the concept of preventing or treating atherosclerosis include: preventing and delaying further progression of severity of so-called atherothrombosis such as ischemic cardiac diseases resulting from atherosclerotic plaque rupture (unstable angina, acute myocardial infarction, acute heart failure, cardiac death) or strokes (including transient cerebral ischemia); preventing occurrence of cardiovascular events of patients having a high risk of developing cardiovascular events (patients with acute coronary artery disease, stroke patients, patients with metabolic disorder, patients with hypertension/obesity/diabetes/hyperlipidemia, etc.) based on anti-atherosclerotic effects; preventing recurrence of ischemic cardiac diseases; preventing primary onset of cardiovascular event; preventing or treating peripheral arterial angiopathy; and the like.

Criteria of diagnosing diabetes have been reported by the Japan Diabetes Society in 1999.

According to this report, diabetes is defined when the fasting glucose level (glucose concentration in venous plasma) is 126 mg/dl or higher, when the level (glucose concentration in venous plasma) 2 hours after 75 g oral glucose tolerance test (75 g OGTT) is 200 mg/dl or higher, or when casual glucose level (glucose concentration in venous plasma) is 200 mg/dl or higher. Moreover, when the condition does not fall into the above-mentioned diabetes but neither fall into “the fasting glucose level (glucose concentration in venous plasma) of less than 110 mg/dl or the level (glucose concentration in venous plasma) 2 hours after 75 g oral glucose tolerance test (75 g OGTT) of less than 140 mg/dl” (normal), it is referred to as a “border-line type”.

In addition, criteria of diabetes have been reported by ADA (American Diabetes Association) and WHO in 1997 and 1998, respectively.

According to these reports, diabetes is defined when the fasting glucose level (glucose concentration in venous plasma) is 126 mg/dl or higher and the level (glucose concentration in venous plasma) 2 hours after the 75 g oral glucose tolerance test is 200 mg/dl or higher.

In addition, according to the above-mentioned report, impaired glucose tolerance is defined when the fasting glucose level (glucose concentration in venous plasma) is less than 126 mg/dl and when the level (glucose concentration in venous plasma) 2 hours after 75 g oral glucose tolerance test is 140 mg/dl or higher but less than 200 mg/dl. Furthermore, according to the report by ADA, a state where the fasting glucose level (glucose concentration in venous plasma) is 110 mg/dl or higher but less than 126 mg/dl is referred to as IFG (Impaired Fasting Glucose). Meanwhile, WHO reported that among such IFG (Impaired Fasting Glucose), a state where the level (glucose concentration in venous plasma) 2 hours after 75 g oral glucose tolerance test is less than 140 mg/dl is referred to as IFG (Impaired Fasting Glycemia).

A compound of the invention may be used as a prophylactic/therapeutic agent for diabetes, border-line type, impaired glucose tolerance, IFG (Impaired Fasting Glucose) and IFG (Impaired Fasting Glycemia) determined according to the above-mentioned criteria. A compound of the invention is also capable of preventing progression from border-line type, impaired glucose tolerance, IFG (Impaired Fasting Glucose) or IFG (Impaired Fasting Glycemia) to diabetes.

A compound of the invention may also be used for secondary prevention and delaying the progression of the above-mentioned various diseases (e.g., cardiovascular events such as myocardial infarction).

By continuously suppressing eicosanoid production for a prolonged time period, a compound of the invention may also be used for preventing or treating inflammatory diseases suggestively associated with prophlogistic eicosanoid, such as asthma, allergic airway hyperresponsiveness, fever, pain production, thrombosis, cerebral infarction, myocardial infarction, cancer, autoimmune encephalomyelitis, pain, renal failure, rheumatism, osteoarthritis, pruritus, atopic dermatitis, rhinitis, inflammatory enteric diseases and Crohn's disease. Furthermore, the compound may improve or suppress enhancement of disorder or abnormality of biological function or physiological action that is causative of various diseases associated with inflammatory reaction, and may be used for primary or secondary prevention and delaying the progression of a disease or a pathological condition resulting therefrom. Examples of such disorders or abnormalities of biological functions and physiological actions include facial flush, pain and itch of skin (including those associated with administration of nicotinic acid derivative preparation, prostacyclin preparation or the like), overactive bladder, disorder or abnormality of cerebral circulatory/renal circulatory autoregulation, circulatory disorder (e.g., peripheral circulation, cerebral circulation, microcirculation, etc.), disorder of blood-brain barrier, salt sensitivity, abnormality of coagulation or fibrinolytic system, abnormality of blood/hemocyte component property (e.g., sickle cell disease, enhanced platelet aggregation, abnormality of erythrocyte deformability, enhanced leukocyte viscosity, increase in blood viscosity, etc.), generation and increased activities of growth factors and cytokines (e.g., PDGF, VEGF, FGF, interleukin, TNF-α, MCP-1, etc.), production and increased invasion of inflammatory cells, increase in free radical generation, acceleration of fatty deposition, endothelial dysfunction, endothelial, cellular and organ damages, edema, morphology alteration of cell such as smooth muscle (morphology alteration into proliferative form or the like), production and enhanced functions of vasoactive substances and thrombus-inducing substances (e.g., catecholamine, endothelin, thromboxane A₂, etc.), abnormal contraction of blood vessel or the like, metabolic abnormality (e.g., serum lipid abnormality, blood glucose abnormality, etc.), overgrowth of cell or the like, and angiogenesis (including abnormal angiopoiesis upon abnormal capillary net formation of outer membrane of atherosclerotic plaque).

Since a compound of the invention has an analgetic effect, it may also be used as an analgesic or a prophylactic/therapeutic drug for pain. Examples of painful diseases include acute pain caused by inflammation, pain associated with chronic inflammation, pain associated with acute inflammation, postoperative pain (pain at an incisional wound, deep pain, vesceral pain, postoperative chronic pain, etc.), muscular ache (muscular ache associated with chronic painful diseases, stiff shoulder, etc.), joint pain, toothache, jaw joint pain, headache (migraine, tension-type headache, headache associated with fever, headache associated with hypertension), vesceral pain (cardiac pain, anginal pain, stomach ache, pain in the kidney, pain in the urinary duct, pain in the bladder), obstetric and gynecologic pain (intermenstrual pain, dysmenorrhea, labor pain), neuralgia (disc herniation, radicular pain, postherpetic neuralgia, trigeminal neuralgia), cancerous pain, reflex sympathetic atrophy and complex regional pain syndrome. The compound of the invention is effective in directly and immediately relieving various pain such as neurogenic pain, cancerous pain and inflammatory pain, and exhibits particularly excellent analgetic effect for patients with low pain threshold and clinical conditions (e.g., hypertension or the like, and complications thereof, etc.).

The content of the compound of the invention in a pharmaceutical composition is generally about 0.01 to about 99.9% by weight, preferably about 0.1 to about 50% by weight of the whole preparation.

A dosage of the compound of the invention is determined by considering age, weight, general health condition, sex, diet, administration time, administration method, excretion rate, combination of drugs, and the condition of the patient's disease under treatment, and/or other factors.

The dosage may vary according to target disease, condition, administration target, administration method and the like. For example, when the compound of the invention is orally administered to an adult as an arteriosclerosis drug, a single dose is generally about 0.01-100 mg/kg weight, preferably 0.05-30 mg/kg weight, more preferably 0.5-10 mg/kg weight, which is administered once to three times a day.

In addition, since the compound of the invention is low toxic and highly safe, it may be administered over a long period of time.

The compound of the invention may be used in combination, for example, with a drug such as an anti-atherosclerotic agent, an anti-thrombotic agent, an anti-heart failure agent, an anti-arrhythmia agent, an anti-hypertensive agent, an agent for treating diabetes, an agent for treating diabetic complications, an HDL-raising agent, an anti-hyperlipidemia agent, an anti-obesity agent, a diuretic, an anti-inflammatory agent, an antigout agent, a chemotherapeutic agent, an immunotherapeutic agent, an osteoporosis drug, an anti-dementia agent, an erectile dysfunction-improving agent, an agent for treating urinary incontinence and an agent for treating urination difficulty (hereinafter, abbreviated as concomitant drugs). These concomitant drugs may be low-molecular compounds, or high-molecular proteins, polypeptides, antibodies, vaccines or the like.

Examples of the above-mentioned “anti-atherosclerotic agent” include Lp-PL A2 inhibitors (e.g., darapladib, rilapladib, etc.), FLAP inhibitors (e.g., AM-103, AM-803, DG-031, etc.), sPLA2 inhibitors (e.g., varespladib), 5-lipoxygenase inhibitors (e.g., VIA-2291, etc.), acyl-coenzyme A: cholesterol acyltransferase (ACAT) inhibitors (e.g., melinamide, avasimibe, eflucimibe, etc.), lipid-rich plaque regression drugs (e.g., compounds described in WO002/06264 and WO03/059900, etc.), reconstituted HDL (e.g., CSL-111, etc.), CTEP inhibitors (e.g., torcetrapib, anacetrapib, dalcetrapib, etc.), MMP inhibitors, chymase inhibitors, SPT inhibitors, ApoA-1 and related molecules thereof (e.g., ApoA-1 Milano, D-4F, L-4F, etc.).

Examples of the above-mentioned “anti-thrombotic agent” include blood coagulation inhibitors (e.g., heparin sodium, heparin calcium, warfarin calcium (warfarin), antithrombin drugs (e.g., argatroban, dabigatran), activated blood coagulation Factor Xa inhibitors (e.g., rivaroxaban, apixaban, edoxaban, YM-150, compounds described in WO02/06234, WO2004/048363, WO2005/030740, WO2005/058823, WO2005/113504 and WO2004/048363), etc.), thrombolytic drugs (e.g., tPA, urokinase, tisokinase, alteplase, nateplase, monteplase, pamiteplase), antiplatelet drugs (e.g., aspirin, sulfinpyrazone (Anturan), dipyridamole (Persantin), ticlopidine (Panaldine), cilostazol (Pletal), GPIIb/IIIa antagonists (e.g., ReoPro, etc.), clopidogrel, prasugrel, ticagrelor, E5555, SHC530348, ethyl icosapentate, beraprost sodium, sarpogrelate hydrochloride, etc.) and the like.

Examples of the above-mentioned “anti-heart failure agent” include inotropic agents (e.g., digitoxin, digoxin, methyldigoxin, lanatoside C, proscillaridin, etc.), α,β stimulants (e.g., epinephrine, norepinephrine, isoproterenol, dopamine, docarpamine, dobutamine, denopamine, etc.), phosphodiesterase inhibitors (e.g., amrinone, milrinone, olprinone hydrochloride, etc.), calcium channel sensitivity augmenting agents (e.g., pimobendan, etc.), nitrate drugs (e.g., nitroglycerin, isosorbide nitrate, etc.), angiotensin-converting enzyme inhibitors (e.g., an angiotensin-converting enzyme inhibitor mentioned below, etc.), angiotensin II antagonist (e.g., an angiotensin II antagonist mentioned below, etc.), β-blockers (e.g., β-blocker mentioned below, etc.), diuretics (e.g., diuretic mentioned below, etc.), ANPs, sGC-activating agents, myosin sensitivity augmenting agents, carperitide, ubidecarenone, vesnarinone, aminophylline and the like.

Examples of the above-mentioned “anti-arrhythmia agents” include sodium channel blockers (e.g., quinidine, procainamide, disopyramide, ajmaline, cibenzoline, lidocaine, diphenylhydantoin, mexiletine, propafenone, flecainide, pilsicainide, phenytoin, etc.), β-blockers (e.g., propranolol, alprenolol, bufetolol, oxprenolol, atenolol, acebutolol, metoprolol, bisoprolol, pindolol, carteolol, arotinolol, etc.), potassium channel blockers (e.g., amiodarone, etc.), calcium channel blockers (e.g., verapamil, diltiazem, etc.) and the like.

Examples of the above-mentioned “anti-hypertensive agent” include angiotensin-converting enzyme inhibitors (e.g., captopril, enalapril, delapril, etc.), angiotensin II antagonists (e.g., candesartan cilexetil, candesartan, azilsartan, azilsartan medoxomil, losartan, losartan potassium, eprosartan, valsartan, telmisartan, irbesartan, tasosartan, olmesartan, olmesartan medoxomil, etc.), calcium antagonists (e.g., manidipine, nifedipine, amlodipine, efonidipine, nicardipine, etc.), β-blockers (e.g., propranolol, nadolol, timolol, nipradilol, bunitrolol, indenolol, penbutolol, carteolol, carvedilol, pindolol, acebutolol, atenolol, bisoprolol, metoprolol, labetalol, amosulalol, arotinolol, etc.), clonidine and the like.

Examples of the above-mentioned “agent for treating diabetes” include insulin preparations (e.g., animal insulin preparations extracted from bovine or swine pancreas; human insulin preparations synthesized by genetic engineering using E. coli or yeast; insulin zinc; protamine insulin zinc; insulin fragments or derivatives (e.g., INS-1), oral insulin preparation), insulin-resistance improving agents (e.g., pioglitazone or salts thereof (preferably, hydrochloride salt), rosiglitazone or salts thereof (preferably, maleate salt), Netoglitazone (MCC-555), Rivoglitazone (CS-011), FK-614, compounds described in WO01/38325, Tesaglitazar (AZ-242), Ragaglitazar (NN-622), Muraglitazar (BMS-298585), Edaglitazone (BM-13-1258), Metaglidasen (MBX-102), Naveglitazar (LY-519818), MX-6054, LY-510929, AMG131(T-131) or salts thereof, THR-0921), α-glucosidase inhibitor (e.g., voglibose, acarbose, miglitol, emiglitate), biguanides (e.g., phenformin, metformin, buformin or salts thereof (e.g., hydrochloride salt, fumarate salt, succinate salt)), insulin secretion promoters (sulphonylurea agents (e.g., tolbutamide, glibenclamide, gliclazide, chlorpropamide, tolazamide, acetohexamide, glyclopyramide, glimepiride, glipizide, glybuzole), repaglinide, nateglinide, mitiglinide or calcium salt hydrates thereof), dipeptidyl peptidase-IV inhibitors (e.g., Vildagliptin (LAF237), P32/98, Sitagliptin (MK-431), alogliptin, P93/01, PT-100, Saxagliptin (BMS-477118), BI1356, GRC8200, MP-513, PF-00734200, PHX1149, SK-0403, ALS2-0426, TA-6666, TS-021, KRP-104, 2-[[6-[(3R)-3-amino-1-piperidinyl]-3,4-dihydro-3-methyl-2,4-dioxo-1(2H)-pyrimidinyl]methyl]-4-fluorobenzonitrile or salts thereof), β3-agonists (e.g., AJ-9677), GPR40 agonists, GLP-1 receptor agonists (e.g., GLP-1, GLP-1MR agent, NN-2211, AC-2993 (exendin-4), BIM-51077, Aib(8,35)hGLP-1(7,37)NH2, CJC-1131], amylin agonists (e.g., pramlintide), phosphotyrosine phosphatase inhibitors (e.g., sodium vanadate), gluconeogenesis inhibitors (e.g., glycogen phosphorylase inhibitors, glucose-6-phosphatase inhibitors, glucagon antagonists), SGLUT (sodium-glucose cotransporter) inhibitors (e.g., T-1095, dapagliflozin, remogliflozin), 11β-hydroxysteroid dehydrogenase inhibitors (e.g., BVT-3498), adiponectin or agonists thereof, IKK inhibitors (e.g., AS-2868), leptin-resistant improving drugs, somatostatin receptor agonists (e.g., compounds described in WO01/25228, WO03/42204, WO98/44921, WO98/45285, WO99/22735, etc.), glucokinase activators (e.g., Ro-28-1675), ACC2 (acetyl-CoA carboxylase 2) inhibitors and the like.

Examples of the above-mentioned “agent for treating diabetic complications” include aldose reductase inhibitors (e.g., tolrestat, epalrestat, zenarestat, zopolrestat, minalrestat, fidarestat, CT-112, ranirestat (AS-3201)), neurotrophic factors and augmenting agents thereof (e.g., NGF, NT-3, BDNF, neurotrophin production/secretion promoters described in WO01/14372 (e.g., 4-(4-chlorophenyl)-2-(2-methyl-1-imidazolyl)-5-(3-(2-methylphenoxy)propyl)oxazole)), PKC inhibitors (e.g., ruboxistaurin mesylate), AGE inhibitors (e.g., ALT946, pimagedine, N-phenacylthiazolium bromide (ALT766), EXO-226, Pyridorin, pyridoxamine), active oxygen scavenging agents (e.g., thioctic acid), cerebral vasodilators (e.g., tiapride, mexiletine), somatostatin receptor agonists (e.g., BIM23190), and apoptosis signal-regulating kinase-1(ASK-1) inhibitors.

Examples of the above-mentioned “HDL-raising agent” include squalene synthetase inhibitors, CETP inhibitors (e.g., torcetrapib, anacetrapib, dalcetrapib, etc.), LPL activators, nicotinic drugs (e.g., nicomol, niceritrol), endothelial lipase inhibitors and the like.

Examples of the above-mentioned “anti-hyperlipidemia agent” include statin compounds as cholesterol synthesis inhibitors (e.g., cerivastatin, pravastatin, simvastatin, lovastatin, rosuvastatin, atorvastatin, fluvastatin, pitavastatin or salts thereof (e.g., sodium salt, etc.) etc.), squalene synthetase inhibitors or fibrate compounds with hypotriglyceride action (e.g., bezafibrate, clofibrate, simfibrate, clinofibrate, etc.), cholesterol absorption inhibitors (e.g., zetia), anion-exchange resins (e.g., cholestyramine), probucol, nicotinic drugs (e.g., nicomol, niceritrol), phytosterols (e.g., soysterol, γ-oryzanol)), fish oil preparations (EPA, DHA, omacor, etc.), PPAR α-agonists, PPAR γ-agonists, PPAR δ-agonists, LXR agonists, FXR antagonists, FXR agonists, DGAT inhibitors, MGAT inhibitors, MTP inhibitors (e.g., lomitapide), nucleic acid drugs including ApoB antisense (e.g., mipomersen) or PCSK9 siRNA antisense oligonucleotides, and the like.

Examples of the above-mentioned “anti-obesity agent” include monoamine uptake inhibitors (e.g., phentermine, sibutramine, mazindol, fluoxetine, tesofensine), serotonin 2C receptor agonists (e.g., lorcaserin), serotonin 6 receptor antagonists, histamine H3 receptors, GABA modulators (e.g., topiramate), neuropeptide Y antagonists (e.g., velneperit), cannabinoid receptor antagonists (e.g., rimonabant, taranabant), ghrelin antagonists, ghrelin receptor antagonists, ghrelin-acylating enzyme inhibitors, opioid receptor antagonists (e.g., GSK-1521498), orexin receptor antagonists, melanocortin 4 receptor agonists, 11β-hydroxysteroid dehydrogenase inhibitors (e.g., AZD-4017), pancreatic lipase inhibitors (e.g., orlistat, cetilistat), β3-agonists (e.g., N-5984), diacylglycerol acyltransferase1 (DGAT1) inhibitors, acetyl CoA carboxylase (ACC) inhibitors, stearate CoA desaturase inhibitors, microsome triglyceride transfer protein inhibitors (e.g., R-256918), Na-glucose cotransport carrier inhibitors (e.g., JNJ-28431754, remogliflozin), NFκ inhibitors (e.g., HE-3286), PPAR agonists (e.g., GFT-505, DRF-11605), phosphotyrosine phosphatase inhibitors (e.g., sodium vanadate, Trodusquemin), GPR119 agonists (e.g., PSN-821), glucokinase activators (e.g., AZD-1656), leptin, leptin derivatives (e.g., metreleptin), CNTFs (ciliary neurotrophic factors), BDNFs (brain-derived neurotrophic factors), cholecystokinin agonists, glucagon-like peptide-1 (GLP-1) preparations (e.g., animal GLP-1 preparations extracted from bovine or swine pancreas; human GLP-1 preparations synthesized by genetic engineering using E. coli or yeast; GLP-1 fragments or derivatives (e.g., exenatide, liraglutide)), amylin preparations (e.g., pramlintide, AC-2307), neuropeptide Y agonists (e.g., PYY3-36, PYY3-36 derivatives, obinepitide, TM-30339, TM-30335), oxyntomodulin preparations: FGF21 preparations (e.g., animal FGF21 preparations extracted from bovine or swine pancreas; human FGF21 preparations synthesized by genetic engineering using E. coli or yeast; FGF21 fragments or derivatives)), appetite suppressors (e.g., P-57) and the like.

Examples of the above-mentioned “diuretics” include xanthine derivatives (e.g., theobromine sodium salicylate, theobromine calcium salicylate, etc.), thiazide preparations (e.g., ethiazide, cyclopenthiazide, trichloromethiazide, hydrochlorothiazide, hydroflumethiazide, bentyl hydrochlorothiazide, penfluthiazide, poly 5 thiazide, methychlothiazide, etc.), anti-aldosterone preparations (e.g., spironolactone, eplerenone, triamterene, etc.), carbonate dehydratase inhibitors (e.g., acetazolamide, etc.), chlorobenzenesulfonamide preparations (e.g., chlortalidone, mefruside, indapamide, etc.), azosemide, isosorbide, ethacrynic acid, piretanide, bumetanide, furosemide and the like.

Examples of the above-mentioned “anti-inflammatory agent” include nonsteroidal anti-inflammatory agents such as acetaminophen, phenacetin, ethenzamide, sulpyrine, antipyrine, migrenin, aspirin, mefenamic acid, flufenamic acid, diclofenac sodium, loxoprofen sodium, phenylbutazone, indomethacin, ibuprofen, ketoprofen, naproxen, oxaprozin, flurbiprofen, fenbufen, pranoprofen, floctafenine, epirizole, tiaramide hydrochloride, zaltoprofen, gabexate mesilate, camostat mesylate, ulinastatin, colchicine, probenecid, sulfinpyrazone, benzbromarone, allopurinol, sodium aurothiomalate, sodium hyaluronate, sodium salicylate, morphine hydrochloride, salicylic acid, atropine, scopolamine, morphine, pethidine, levorphanol, ketoprofen, naproxen, oxymorphone and salts thereof, and the like.

Examples of the above-mentioned “antigout agent” include febuxostat, allopurinol, probenecid, colchicine, benzbromarone, febuxostat, citric salt and the like.

Examples of the above-mentioned “chemotherapeutic agent” include alkylating agents (e.g., cyclophosphamide, ifosfamide, etc.), metabolic antagonists (e.g., methotrexate, 5-fluorouracil, etc.), anticancerous antibiotics (e.g., mitomycin, adriamycin, etc.), plant-derived anticancer agents (e.g., vincristine, vindesine, taxol, etc.), cisplatin, carboplatin, etoposide and the like. In particular, 5-fluorouracil derivatives furtulon, neofurtulon and the like are preferable.

Examples of the above-mentioned “immunotherapeutic agent” include microbial or bacterial components (e.g., muramyldipeptide derivatives, picibanil, etc.), polysaccharides with immunological-enhancing activity (e.g., lentinan, schizophyllan, krestin, etc.), cytokines obtained through genetic engineering procedure (e.g., interferon, interleukin (IL), etc.), colony-stimulating factors (e.g., granulocyte colony-stimulating factors, erythropoietin, etc.) and the like. In particular, IL-1, IL-2, IL-12 and the like are preferable.

Examples of the above-mentioned “osteoporosis drug” include alfacalcidol, calcitriol, elcaltonin, calcitonin salmon, estriol, ipriflavone, pamidronate disodium, alendronate sodium hydrate, incadronate disodium and the like.

Examples of the above-mentioned “an antidementia agent” include tacrine, donepezil, rivastigmine, galantamine and the like.

Examples of the above-mentioned “erectile dysfunction improving agent” include apomorphine, PDE5 (phosphodiesterase5) inhibitors (e.g., sildenafil citrate) and the like.

Examples of the above-mentioned “agent for treating urinary incontinence” include flavoxate hydrochloride, oxybutynin hydrochloride, propiverine hydrochloride and the like.

Examples of the above-mentioned “agent for treating urination difficulty” include acetylcholinesterase inhibitors (e.g., distigmine) and the like.

Moreover, examples of concomitant drugs include prostacyclin preparations/derivatives (e.g., beraprost, epoprostenol, iloprost, treprostinil, etc.), prostaglandin preparations/derivatives (e.g., enprostil, alprostadil, limaprost, misoprostol, ornoprostil, etc.), anti-asthma drugs (e.g., salmeterol, fluticasone, montelukast), rheumatoid arthritis agents (e.g., etanercept, infliximab, adalimumab), nerve regeneration promoters (e.g., Y-128, VX-853, prosaptide), antidepressants (e.g., desipramine, amitriptyline, imipramine), antiepilepsy drugs (e.g., lamotrigine), antiarrhythmic drugs (e.g., mexiletine), acetylcholine receptor ligands (e.g., ABT-594), endothelin receptor antagonists (e.g., bosentan, ABT-627), monoamine uptake inhibitors (e.g., tramadol), narcotic analgesics (e.g., morphine), GABA receptor agonists (e.g., gabapentin), α2 receptor agonists (e.g., clonidine), local analgesics (e.g., capsaicin), antianxiety drugs (e.g., benzodiazepine), dopamine agonists (e.g., apomorphine), midazolam, ketoconazole and the like.

The administration period of the above-mentioned concomitant drug is not limited; the compound of the invention and a concomitant drug may be administered to an administration target either simultaneously or with time difference. A dosage of a concomitant drug is pursuant to clinically employed dosages, and may appropriately be selected based on the administration target, administration route, disease, combination or the like.

In addition, two or more of these concomitant drugs may be combined in an appropriate proportion. In this case, the administration period of the compound of the invention and the concomitant drugs is not limited as long as the compound of the invention is combined with the concomitant drugs upon administration.

Examples of such administration modes include: (1) administrating a single-unit preparation obtained by formulating the compound of the invention together with the concomitant drug, (2) simultaneously administrating two types of preparations via the same administration route, where the preparations are obtained by separately formulating the compound of the invention and the concomitant drug, (3) administrating two types of preparations at different times via the same administration route, where the preparations are obtained by separately formulating the compound of the invention and the concomitant drug, (4) simultaneously administering two types of preparations via different administration routes, where the preparations are obtained by separately formulating the compound of the invention and the concomitant drug, (5) administrating two types of preparations at different times via different administration routes, where the preparations are obtained by separately formulating the compound of the invention and the concomitant drug (for example, administering in the order of the compound of the invention→concomitant drug, or vice versa). A dosage of a concomitant drug may be appropriately selected based on clinically employed doses. Furthermore, the ratio of the compound of the invention and a concomitant drug may appropriately be selected depending on the administration target, administration route, target disease, condition, combination and the like. For example, when the administration target is human, 0.01-100 parts by weight of a concomitant drug is used to a part by weight of the compound of the invention.

A compound of the invention may be orally or parenterally administered directly or by adding a pharmacologically acceptable carrier.

A medical drug of the present invention comprising the compound of the invention may be safely administered orally or parenterally (e.g., intravenous, intramuscular, subcutaneous, intraorgan, intranasal, intradermal, ocular, intracerebral, intrarectal, vaginal, intraperitoneal or intratumoral administration, administration proximal to tumor or directly to the lesion), for example, as a tablet (including sugar-coated tablet, film-coated tablet, sublingual tablet, orally-disintegrating tablet, buccal tablet or the like), a pill, a powdered agent, a granular agent, a capsule (including soft capsule, microcapsule), a lozenge, syrup, a liquid agent, an emulsion, a suspension, a controlled-release preparation (e.g., quick-release preparation, sustained-release preparation, sustained-release microcapsule), aerosol, a film agent (e.g., orally-disintegrating film, film applicable to oral mucosa), an injection (e.g., subcutaneous injection, intravenous injection, intramuscular injection, intraperitoneal injection), drops, a transdermally absorbed preparation, an ointment, lotion, a patch, a suppository (e.g., rectal suppository, vaginal suppository), pellets, a nasal agent, a transpulmonary agent (inhalation), eye-drops or the like, by administering the compound of the invention alone or together with a pharmacologically acceptable carrier according to a method known per se as a method for producing a drug preparation (e.g., methods described in the Japanese Pharmacopoeia, etc.).

As need arises, the compound of the invention may be produced into any of the above-mentioned formulations by appropriately adding an appropriate amount of an excipient, a binder, a disintegrating agent, a lubricant, a sweetening agent, a surfactant, a suspending agent, an emulsifier or the like generally employed in the formulation field.

For example, when the compound of the invention is produced into a tablet, it may be added with an excipient, a binder, a disintegrating agent, a lubricant or the like. When the compound of the invention is produced into a pill or a granular agent, it may be added with an excipient, a binder, a disintegrating agent or the like. Moreover, when the compound of the invention is produced into a powdered agent or a capsule, it may be added with an excipient or the like. When the compound of the invention is produced into syrup, it may be added with a sweetening agent or the like. When the compound of the invention is produced into an emulsion or a suspension, it may be added with a suspending agent, a surfactant, an emulsifier or the like.

Examples of an excipient include lactose, white sugar, glucose, starch, sucrose, microcrystalline cellulose, powdered glycyrrhiza, mannitol, sodium hydrogen carbonate, calcium phosphate, calcium sulfate and the like.

Examples of a binder include a 5 to 10% by weight starch glue solution, a 10 to 20% by weight gum arabic or gelatin solution, a 1 to 5% by weight tragacanth solution, a carboxymethyl cellulose solution, a sodium alginate solution, glycerin and the like.

Examples of a disintegrating agent include starch, calcium carbonate and the like.

Examples of a lubricant include magnesium stearate, stearic acid, calcium stearate, purified talc and the like.

Examples of a sweetening agent include glucose, fructose, invert sugar, sorbitol, xylitol, glycerin, simple syrup and the like.

Examples of a surfactant include sodium lauryl sulfate, Polysorbate 80, sorbitan mono-fatty acid ester, Polyoxyl 40 stearate and the like.

Examples of a suspension include gum arabic, sodium alginate, sodium carboxymethyl cellulose, methylcellulose, bentonite and the like.

Examples of an emulsifier include gum arabic, tragacanth, gelatin and Polysorbate 80.

In addition, when the compound of the invention is produced into the above-mentioned preparations, if necessary, it may be added with an appropriate amount of a colorant, a preservative, an aromatic substance, a flavoring substance, a stabilizer, a viscous agent or the like generally used in the formulation field.

When the compound of the invention is parenterally administered, it is generally administered in a liquid (e.g., injection) form. Although its single-unit dosage varies depending on the administration target, target organ, conditions, administration method or the like, in the case of injection, for example, a dosage of usually about 0.01 mg to about 100 mg, preferably about 0.01 to about 50 mg, more preferably about 0.01 to about 20 mg per kg weight may conveniently be administered by intravenous injection. Other than intravenous injection, subcutaneous injection, intradermal injection, intramuscular injection, drip injection and the like are available. As a prolonged preparation, iontophoresis transdermal agents and the like are available. These injections may be prepared according to a method known per se, namely, by dissolving, suspending or emulsifying Compound (I) in a sterile aqueous or oily solution. Examples of an aqueous solution for injection include physiological saline, glucose and isotonic solutions including other supplements (e.g., D-sorbitol, D-mannitol, sodium chloride, etc.), which may be used with an appropriate solubilizer such as alcohol (e.g., ethanol), polyalcohol (e.g., propylene glycol, polyethyleneglycol), nonionic surfactant (e.g., Polysorbate 80, HCO-50) or the like. Examples of oily solutions include sesame oil, soybean oil and the like, which may be used with a solubilizer such as benzyl benzoate, benzyl alcohol or the like. In addition, a buffer (e.g., phosphate buffer, sodium acetate buffer), a soothing agent (e.g., benzalkonium chloride, procaine hydrochloride, etc.), a stabilizer (e.g., human serum albumin, polyethylene glycol, etc.), a preservative (e.g., benzyl alcohol, phenol, etc.) or the like may also be added. The prepared injection is usually loaded into an ample.

Herein below, the method for producing the compounds of the present invention will be explained.

Compound (I) may be prepared, for example, by the method described below or a method pursuant thereto.

In the reaction formulas described below, individual raw compounds may be in the form of salt if it does not inhibit the reaction. As such salts, those exemplified above as salts of the compound represented by formula (I) may be used.

When specific preparation methods are not described herein, raw compounds are easily available in the market or may be prepared by a method known per se or a method pursuant thereto.

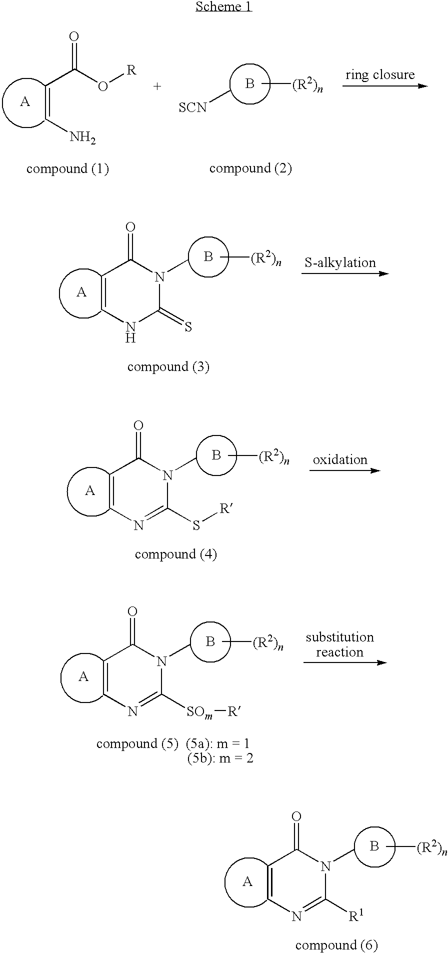

[wherein, each symbol has the same meaning as defined in the above and R represents C₁₋₆ alkyl.]

Compound (6) can be produced according to the pathway described in Scheme 1. That is, it can be produced from compound (1) via compound (3), compound (4), and substitution reaction of compound (5).

Compound (3) can be produced according to the ring closure reaction between compound (1) and compound (2) which is carried out in the presence of a base. Specifically, compound (2) is used in an amount of about 1.0 to 5.0 mol, preferably about 1.0 to 2.0 mol, relative to 1 mol of compound (1). The base includes inorganic bases such as sodium hydroxide, potassium hydroxide, barium hydroxide and the like, basic salts such as sodium carbonate, potassium carbonate and the like, metal alkoxides such as sodium methoxide, sodium ethoxide, potassium tert-butoxide and the like, metal hydrides such as sodium hydride, potassium hydride and the like, and organic bases such as triethylamine, imidazole, formamidine and the like, and it is used in an amount of about 1.0 to 10.0 mol, preferably about 1.0 to 5.0 mol, relative to 1.0 mol of compound (1). The present reaction is preferably carried out by using a solvent inert to the reaction. The preferred solvent includes, but not particularly limited to as long as the reaction proceeds, halogenated hydrocarbons such as dichloromethane, chloroform, carbon tetrachloride, 1,2-dichloroethane and the like, aromatic hydrocarbons such as benzene, toluene and the like, ethers such as tetrahydrofuran, dioxane, 1,2-dimethoxyethane and the like, amides such as N,N-dimethylformamide, N,N-dimethylacetamide and the like, nitriles such as acetonitrile, propionitrile and the like, sulfoxides such as dimethyl sulfoxide and the like, phosphorous acid amides such as N,N,N′,N′,N″,N″-hexamethylphosphoric triamide and the like, or a mixture solvent thereof. The reaction time is generally 1 hr to 60 hr, preferably 1 hr to 24 hr. The reaction temperature is generally −10 to 200° C., preferably 0 to 100° C. The resulting compound (3) may be used for the next step in the state of a reaction solution directly or a crude product. Alternatively, according to a typical process, compound (3) can be isolated from the reaction mixture and also can be easily purified with a separation means such as washing, recrystallization, distillation, chromatography and the like.

Compound (4) can be produced according to the S-alkylation reaction of compound (3) using a base and various alkylating agents. Specifically, the base is used in an amount of 1.0 to 10.0 mol, preferably 1.0 to 5.0 mol, and the alkylating agent is used in an amount of 1.0 to 20.0 mol, preferably 1.0 to 10.0 mol, relative to 1 mol of compound (3). The base includes inorganic bases such as sodium hydroxide, potassium hydroxide, barium hydroxide and the like, basic salts such as sodium hydrogen carbonate, sodium carbonate, potassium carbonate and the like, metal alkoxides such as sodium methoxide, sodium ethoxide, potassium tert-butoxide and the like, metal hydrides such as sodium hydride, potassium hydride and the like, and organic bases such as triethylamine, imidazole, formamidine and the like. The alkylating agent includes various halogenated alkyls such as alkyl chloride, alkyl bromide, alkyl iodide and the like and derivatives thereof, sulfonic acid esters such as p-toluenesulfonic acid ester, methanesulfonic acid ester and the like, and sulfuric acid esters such as dimethyl sulfate and the like. The present reaction is preferably carried out by using a solvent inert to the reaction. The solvent includes, but not particularly limited to as long as the reaction proceeds, halogenated hydrocarbons such as dichloromethane, chloroform, carbon tetrachloride, 1,2-dichloroethane and the like, alcohols such as methanol, ethanol, propanol, 1,1-dimethylethanol and the like, aromatic hydrocarbons such as benzene, toluene and the like, ethers such as tetrahydrofuran, dioxane, 1,2-dimethoxyethane and the like, amides such as N,N-dimethylformamide, N,N-dimethylacetamide and the like, nitriles such as acetonitrile, propionitrile and the like, sulfoxides such as dimethyl sulfoxide and the like, water, or a mixture solvent thereof. The reaction time is generally 15 min to 60 hr, preferably 15 min to 24 hr. The reaction temperature is generally −10 to 200° C., preferably 0 to 150° C. The resulting compound (4) may be used for the next step in the state of a reaction solution directly or a crude product. Alternatively, according to a typical process, compound (4) can be isolated from the reaction mixture and in particular can be easily purified with a separation means such as washing, recrystallization, distillation, chromatography and the like.

Compound (5) is produced according to the oxidation reaction of compound (4). Specifically, an oxidizing agent includes peracids such as hydrogen peroxide, Oxone (registered trademark) monopersulfate compound, peracetic acid, perbenzoic acid, metachloroperbenzoic acid and the like, oxoacids and salts thereof such as hypochlorous acid, periodic acid and the like, metal oxoacids and salts thereof such as chromic acid and the like, or other oxidizing agent. It is used in an amount of 1.0 to 30.0 mol, preferably 1.0 to 3.0 mol, relative to 1.0 mol of compound (4). The present reaction is preferably carried out by using a solvent inert to the reaction. The preferred solvent includes, but not particularly limited to as long as the reaction proceeds, alcohols such as methanol, ethanol, propanol, 1,1-dimethylethanol and the like, aromatic hydrocarbons such as benzene, toluene and the like, amides such as N,N-dimethylformamide, N,N-dimethylacetamide and the like, nitriles such as acetonitrile, propionitrile and the like, sulfoxides such as dimethyl sulfoxide and the like, carboxylic acids such as acetic acid and the like, water, or a mixture solvent thereof. The reaction time is generally 1 hr to 60 hr, preferably 1 hr to 5 hr. The reaction temperature is generally −10 to 200° C., preferably 0 to 150° C. A reaction product is obtained as a single compound of either compound (5a) or compound (5b), or as their mixture, and it may be used for the next step in the state of a reaction solution directly or a crude product. Alternatively, according to a typical process, a single compound of compound (5a) or compound (5b) can be isolated from the reaction mixture and in particular can be easily purified with a separation means such as washing, recrystallization, distillation, chromatography and the like.

Compound (6) can be produced from compound (5) by the substitution reaction using the base and various nucleophilic agents. Specifically, the base is used in an amount of 1.0 to 20.0 mol, preferably 1.0 to 10.0 mol, and the nucleophilic agent is used in an amount of 1.0 to 100.0 mol, preferably 1.0 to 10.0 mol, relative to 1 mol of compound (5). The base includes inorganic bases such as sodium hydroxide, potassium hydroxide, barium hydroxide and the like, basic salts such as sodium carbonate, potassium carbonate and the like, metal alkoxides such as sodium methoxide, sodium ethoxide, potassium tert-butoxide and the like, metal hydrides such as sodium hydride, potassium hydride and the like, and organic base such as 1,8-diazabicyclo[5.4.0]undeca-7-en, 1,4-diazabicyclo[2.2.2]octane and the like. The nucleophilic agent includes alcohols such as methanol, ethanol, propanol, 1,1-dimethylethanol and the like, various phenol derivatives having an aromatic hydroxyl group, organic thiols such as ethanethiol, thioglycolic acid amide and the like, various aromatic thiol derivatives such as thiophenol and the like, organic bases such as methylamine, ethylamine and the like, various aromatic amines such as aniline and the like, water and the like. The base can be also used as a nucleophilic agent, if necessary. The reaction is preferably carried out without any solvent or by using a solvent which is inert to the reaction. The preferred solvent includes, but not particularly limited to as long as the reaction proceeds, alcohols such as methanol, ethanol, propanol and the like, aromatic hydrocarbons such as benzene, toluene and the like, saturated hydrocarbons such as cyclohexane, hexane and the like, ethers such as tetrahydrofuran, dioxane, 1,2-dimethoxyethane and the like, amides such as N,N-dimethylformamide, N,N-dimethylacetamide and the like, nitriles such as acetonitrile, propionitrile and the like, ketones such as acetone, methylethyl ketone and the like, sulfoxides such as dimethyl sulfoxide and the like, water, or a mixture solvent thereof The reaction time is generally 10 min to 24 hr, preferably 10 min to 12 hr. The reaction temperature is generally −10 to 200° C., preferably 0 to 100° C. The resulting compound (6) may be used for the next step in the state of a reaction solution directly or a crude product. Alternatively, according to a typical process, compound (6) can be isolated from the reaction mixture and in particular can be easily purified with a separation means such as washing, recrystallization, distillation, chromatography and the like.

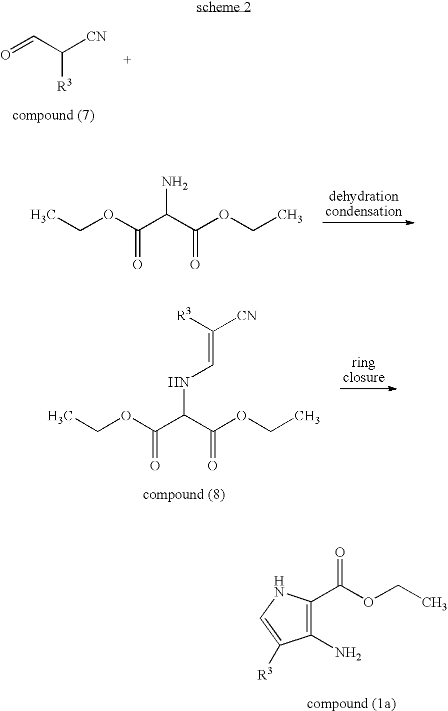

[wherein, R³ has the same meaning as defined in the above.]

Compound (1a) can be produced according to a known method, for example, by a method described in Journal of Organic Chemistry (J. Org. Chem.), vol. 62, page 8071 (1997) or ibid, vol. 64, page 8411 (1999), or a method pursuant thereto. Specifically, compound (1a) is produced from compound (8) by the ring closure reaction using the base (Scheme 2). The base includes inorganic bases such as sodium hydroxide, potassium hydroxide, barium hydroxide and the like, basic salts such as sodium carbonate, potassium carbonate and the like, metal alkoxides such as sodium methoxide, sodium ethoxide, potassium tert-butoxide and the like, and metal hydrides such as sodium hydride, potassium hydride and the like, and it is used in an amount of about 1.0 to 10.0 mol, preferably about 1.0 to 5.0 mol, relative to 1 mol of compound (8). The present reaction is preferably carried out by using a solvent inert to the reaction. The preferred solvent includes, but not particularly limited to as long as the reaction proceeds, alcohols such as methanol, ethanol, propanol, 1,1-dimethylethanol and the like, aromatic hydrocarbons such as benzene, toluene and the like, ethers such as tetrahydrofuran, dioxane, 1,2-dimethoxyethane and the like, amides such as N,N-dimethylformamide, N,N-dimethylacetamide and the like, nitriles such as acetonitrile, propionitrile and the like, sulfoxides such as dimethyl sulfoxide and the like, or a mixture solvent thereof. The reaction time is generally 1 hr to 60 hr, preferably 1 hr to 24 hr. The reaction temperature is generally −10 to 200° C., preferably 0 to 130° C. The resulting compound (1a) may be used for the next step in the state of a reaction solution directly or a crude product. Alternatively, according to a typical process, compound (1a) can be isolated from the reaction mixture and in particular can be easily purified with a separation means such as washing, recrystallization, distillation, chromatography and the like.

Further, compound (8) can be also produced according to a known method, for example, by a method described in Journal of Organic Chemistry (J. Org. Chem.), vol. 62, page 8071 (1997) or ibid, vol. 64, page 8411 (1999), or a method pursuant thereto. Specifically, it is produced from the dehydration condensation reaction between compound (7) and diethyl 2-aminomalonate (Scheme 2). Diethyl 2-aminomalonate is used in an amount of about 1.0 to 10.0 mol, preferably about 1.0 to 5.0 mol, relative to 1 mol of compound (7). The present reaction is preferably carried out by using a solvent inert to the reaction. The preferred solvent includes, but not particularly limited to as long as the reaction proceeds, halogenated hydrocarbons such as dichloromethane, chloroform, carbon tetrachloride, 1,2-dichloroethane and the like, alcohols such as methanol, ethanol, propanol, 1,1-dimethylethanol and the like, aromatic hydrocarbons such as benzene, toluene and the like, ethers such as tetrahydrofuran, dioxane, 1,2-dimethoxyethane and the like, amides such as N,N-dimethylformamide, N,N-dimethylacetamide and the like, nitriles such as acetonitrile, propionitrile and the like, sulfoxides such as dimethyl sulfoxide and the like, or a mixture solvent thereof. The reaction time is generally 1 hr to 60 hr, preferably 1 hr to 24 hr. The reaction temperature is generally −10 to 200° C., preferably 0 to 150° C. The present reaction can employ an acid catalyst, if necessary. The acid catalyst includes mineral acids such as hydrochloric acid, sulfuric acid and the like, Lewis acids such as boron trichloride, boron tribromide and the like, organic acids such as trifluoroacetic acid, p-toluenesulfonic acid and the like. The resulting compound (8) may be used for the next step in the state of a reaction solution directly or a crude product. Alternatively, according to a typical process, compound (8) can be isolated from the reaction mixture and in particular can be easily purified with a separation means such as washing, recrystallization, distillation, chromatography and the like.

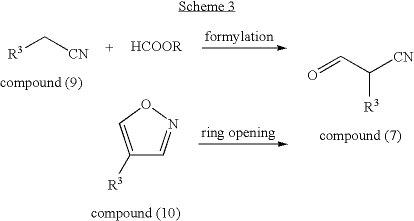

[wherein, R³ and R have the same meanings as defined in the above.]

Further, compound (7) can be produced according to a known method, for example, by a method described in Journal of Medicinal Chemistry (J. Med. Chem.), vol. 36, page 55 (1993), or a method pursuant thereto. Specifically, it is produced from the α-formylation of compound (9) using the base and formic acid ester (Scheme 3). The base includes inorganic bases such as sodium hydroxide, potassium hydroxide, barium hydroxide and the like, basic salts such as sodium carbonate, potassium carbonate and the like, metal alkoxides such as sodium methoxide, sodium ethoxide, potassium tert-butoxide and the like, and metal hydrides such as sodium hydride, potassium hydride and the like, and it is used in an amount of about 1.0 to 10.0 mol, preferably about 1.0 to 5.0 mol, relative to 1 mol of compound (9). The formic acid ester are used, including esters such as methyl formate, ethyl formate and the like, and they are used in an amount of about 1.0 to 10.0 mol, preferably about 1.0 to 5.0 mol, relative to 1 mol of compound (9). The present reaction is preferably carried out by using a solvent inert to the reaction. The preferred solvent includes, but not particularly limited to as long as the reaction proceeds, alcohols such as methanol, ethanol, propanol, 1,1-dimethylethanol and the like, aromatic hydrocarbons such as benzene, toluene and the like, ethers such as tetrahydrofuran, dioxane, 1,2-dimethoxyethane and the like, amides such as N,N-dimethylformamide, N,N-dimethylacetamide and the like, sulfoxides such as dimethyl sulfoxide and the like, or a mixture solvent thereof. The reaction time is generally 1 hr to 60 hr, preferably 1 hr to 24 hr. The reaction temperature is generally −10 to 200° C., preferably 0 to 150° C.

Further, compound (7) can be also produced according to another known method, for example, by a method described in Journal of Organic Chemistry (J. Org. Chem.), vol. 64, page 8411 (1999), or a method analogous thereto (Scheme 3). Specifically, it can be produced from the ring opening reaction of compound (10) using the base. The base includes inorganic bases such as sodium hydroxide, potassium hydroxide, barium hydroxide and the like, basic salts such as sodium carbonate, potassium carbonate and the like, metal alkoxides such as sodium methoxide, sodium ethoxide, potassium tert-butoxide and the like, and metal hydrides such as sodium hydride, potassium hydride and the like, and it is used in an amount of about 1.0 to 10.0 mol, preferably about 1.0 to 5.0 mol, relative to 1 mol of compound (10). The present reaction is preferably carried out by using a solvent inert to the reaction. The preferred solvent includes, but not particularly limited to as long as the reaction proceeds, alcohols such as methanol, ethanol, propanol, 1,1-dimethylethanol and the like, aromatic hydrocarbons such as benzene, toluene and the like, ethers such as tetrahydrofuran, dioxane, 1,2-dimethoxyethane and the like, amides such as N,N-dimethylformamide, N,N-dimethylacetamide and the like, sulfoxides such as dimethyl sulfoxide and the like, or a mixture solvent thereof. The reaction time is generally 1 hr to 60 hr, preferably 1 hr to 24 hr. The reaction temperature is generally −10 to 200° C., preferably 0 to 150° C. The resulting compound (7) may be used for the next step in the state of a reaction solution directly or a crude product. Alternatively, according to a typical process, compound (7) can be isolated from the reaction mixture and in particular can be easily purified with a separation means such as washing, recrystallization, distillation, chromatography and the like.

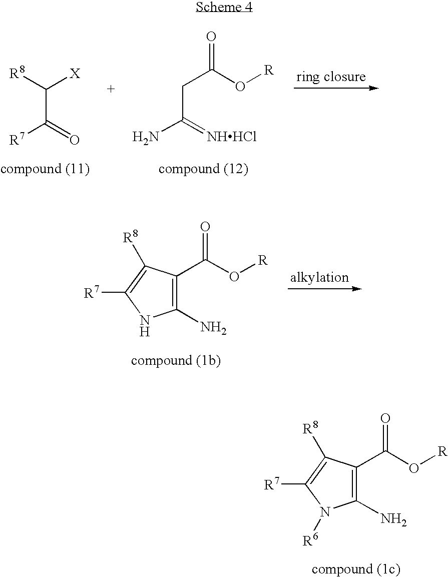

[wherein, X represents a halogen atom and other symbols have the same meanings as defined in the above.]

Compound (1c) can be produced according to a known method, for example, by a method described in Synthesis, page 272 (1987), or a method pursuant thereto (Scheme 4). Specifically, compound (1c) can be produced by N-alkylation of compound (1b), which is produced from compound (11) and compound (12), (provided that, each R⁷ and R⁸ is a hydrogen or a hydrocarbon).

Compound (1b) can be produced by the ring closure reaction of compound (11) and compound (12) using the base. Specifically, compound (12) is used in an amount of about 1.0 to 10.0 mol, preferably about 1.0 to 5.0 mol, relative to 1 mol of compound (11). The base includes inorganic bases such as sodium hydroxide, potassium hydroxide, barium hydroxide and the like, basic salts such as sodium carbonate, potassium carbonate and the like, metal alkoxides such as sodium methoxide, sodium ethoxide, potassium tert-butoxide and the like, metal hydrides such as sodium hydride, potassium hydride and the like, and organic bases such as triethylamine, imidazole, formamidine and the like, and it is used in an amount of about 1.0 to 10.0 mol, preferably about 1.0 to 5.0 mol, relative to 1 mol of compound (11). The present reaction is preferably carried out by using a solvent inert to the reaction. The preferred solvent includes, but not particularly limited to as long as the reaction proceeds, alcohols such as methanol, ethanol, propanol, 1,1-dimethylethanol and the like, aromatic hydrocarbons such as benzene, toluene and the like, ethers such as tetrahydrofuran, dioxane, 1,2-dimethoxyethane and the like, amides such as N,N-dimethylformamide, N,N-dimethylacetamide and the like, sulfoxides such as dimethyl sulfoxide and the like, nitriles such as acetonitrile, propionitrile and the like, or a mixture solvent thereof. The reaction time is generally 1 hr to 60 hr, preferably 1 hr to 24 hr. The reaction temperature is generally −10 to 200° C., preferably 0 to 100° C. The resulting compound (1b) may be used for the next step in the state of a reaction solution directly or a crude product. Alternatively, according to a typical process, compound (1b) can be isolated from the reaction mixture and in particular can be easily purified with a separation means such as washing, recrystallization, distillation, chromatography and the like. Compound (12) can be also produced according to a known method, for example, by a method described in Chem. Pharm. Bull., vol. 43, page 788 (1995), or a method pursuant thereto.

Compound (1c) can be produced by N-alkylation reaction of compound (1b) using the base. Specifically, the alkylating agent is used in an amount of about 1.0 to 20.0 mol, preferably about 1.0 to 10.0 mol, and the base is used in an amount of about 1.0 to 10.0 mol, preferably about 1.0 to 5.0 mol, relative to 1 mol of compound (1b). The alkylating agent includes various halogenated alkyls such as methyl iodide, ethyl iodide, propyl iodide and the like, alkyl sulfates such as dimethyl sulfate, diethyl sulfate and the like, sulfonic acid alkyl esters such as p-toluenesulfonic acid methyl ester, methanesulfonic acid methyl ester and the like. The base includes inorganic bases such as sodium hydroxide, potassium hydroxide, barium hydroxide and the like, basic salts such as sodium carbonate, potassium carbonate and the like, and metal alkoxides such as sodium methoxide, sodium ethoxide, potassium tert-butoxide and the like. The present reaction is preferably carried out by using a solvent inert to the reaction. The preferred solvent includes, but not particularly limited to as long as the reaction proceeds, alcohols such as methanol, ethanol, propanol, 1,1-dimethylethanol and the like, aromatic hydrocarbons such as benzene, toluene and the like, ethers such as tetrahydrofuran, dioxane, 1,2-dimethoxyethane and the like, amides such as N,N-dimethylformamide, N,N-dimethylacetamide and the like, sulfoxides such as dimethyl sulfoxide and the like, or a mixture solvent thereof. The reaction time is generally 1 hr to 60 hr, preferably 1 hr to 24 hr. The reaction temperature is generally −10 to 200° C., preferably 0 to 150° C. The resulting compound (1c) may be used for the next step in the state of a reaction solution directly or a crude product. Alternatively, according to a typical process, compound (1c) can be isolated from the reaction mixture and in particular can be easily purified with a separation means such as washing, recrystallization, distillation, chromatography and the like.

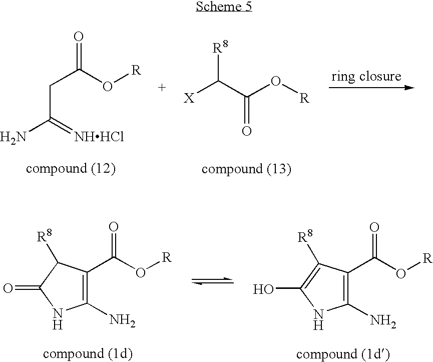

[wherein, each symbol has the same meaning as defined in the above.]

Compound (1d) and compound (1d′), which is the tautomer of compound (1d), can be produced by the ring closure reaction of compound (12) and compound (13) using the base (Scheme 5). Specifically, compound (13) is used in an amount of about 1.0 to 10.0 mol, preferably about 1.0 to 3.0 mol, relative to 1 mol of compound (12). The base is used in an amount of about 1.0 mol to 10.0 mol, preferably about 1.0 mol to 3.0 mol, relative to 1 mol of compound (12). As for the compound (13), ethyl bromoacetate, isopropyl bromoacetate, methyl 2-bromopropionate and the like are used. Further, the base includes inorganic bases such as sodium hydroxide, potassium hydroxide, barium hydroxide and the like, basic salts such as sodium hydrogen carbonate, sodium carbonate, potassium carbonate and the like, metal alkoxides such as sodium methoxide, sodium ethoxide, potassium tert-butoxide and the like, metal hydrides such as sodium hydride, potassium hydride and the like, and organic bases such as triethylamine, imidazole, formamidine and the like. The present reaction is preferably carried out by using a solvent inert to the reaction. The preferred solvent includes, but not particularly limited to as long as the reaction proceeds, halogenated hydrocarbons such as dichloromethane, chloroform, carbon tetrachloride, 1,2-dichloroethane and the like, alcohols such as methanol, ethanol, propanol, 1,1-dimethylethanol and the like, aromatic hydrocarbons such as benzene, toluene and the like, ethers such as tetrahydrofuran, dioxane, 1,2-dimethoxyethane and the like, amides such as N,N-dimethylformamide, N,N-dimethylacetamide and the like, sulfoxides such as dimethyl sulfoxide and the like, nitriles such as acetonitrile, propionitrile and the like, water or a mixture solvent thereof. The reaction time is generally 30 min to 24 hr, preferably 30 min to 12 hr. The reaction temperature is generally −10 to 200° C., preferably 0 to 150° C. A reaction product is obtained as a single compound of either compound (1d) or compound (1d′), which is the tautomer of compound (1d), or as their mixture, and it may be used for the next step in the state of a reaction solution directly or a crude product. Alternatively, according to a typical process, a single compound, either of compound (1d) or compound (1d′), or a their mixture can be isolated from the reaction mixture and in particular can be purified with a separation means such as washing, recrystallization, distillation, chromatography and the like.

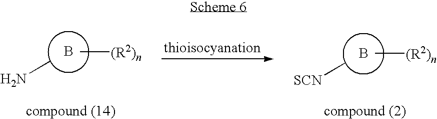

[wherein, each symbol has the same meaning as defined in the above.]

Compound (2) can be produced by thioisocyanation of compound (14). Specifically, the thioisocyanating agent is used for the reaction in an amount of about 1.0 to 5.0 mol, preferably about 1.0 to 2.0 mol, relative to 1 mol of compound (14). The thioisocyanating agent includes thiophosgene, 1,1′-carbonothioyldipyridin-2(1H)-one, di-2-pyridyl thionocarbonate, 1,1′-thiocarbonyl diimidazole and the like. When thiophosgene is used for the present reaction, the reaction can be carried out in the presence of a deacidifying agent to remove the released halogenated hydrogens from the reaction system. For example, the deacidifying agent can be added, including basic salts such as sodium carbonate, potassium carbonate, sodium hydrogen carbonate and the like, aromatic amines such as pyridine, lutidine and the like, tertiary amines such as triethylamine, tripropylamine, tributylamine, cyclohexyldimethylamine, 4-dimethylaminopyridine, N,N-dimethylaniline, N-methylpiperidine, N-methylpyrrolidine, N-methylmorpholine and the like. The present reaction is preferably carried out by using a solvent inert to the reaction. The preferred solvent includes, but not particularly limited to as long as the reaction proceeds, halogenated hydrocarbons such as dichloromethane, chloroform, carbon tetrachloride, 1,2-dichloroethane and the like, alcohols such as methanol, ethanol, propanol and the like, aromatic hydrocarbons such as benzene, toluene and the like, saturated hydrocarbons such as cyclohexane, hexane and the like, ethers such as tetrahydrofuran, dioxane, 1,2-dimethoxyethane and the like, amides such as N,N-dimethylformamide, N,N-dimethylacetamide and the like, nitriles such as acetonitrile, propionitrile and the like, ketones such as acetone, methyl ethyl ketone and the like, sulfoxides such as dimethyl sulfoxide and the like, water or a mixture solvent thereof. The reaction time is generally 10 min to 60 hr, preferably 15 min to 12 hr. The reaction temperature is generally −10 to 200° C., preferably 0 to 120° C. The resulting compound (2) may be used for the next step in the state of a reaction solution directly or a crude product. Alternatively, according to a typical process, compound (2) can be isolated from the reaction mixture and in particular can be easily purified with a separation means such as recrystallization, distillation, chromatography and the like.

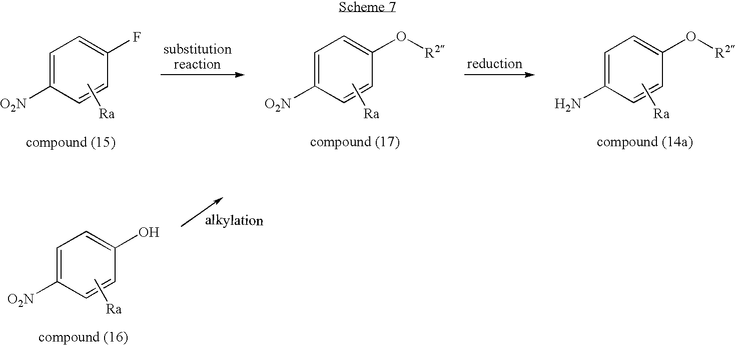

[wherein, Ra has the same meaning as defined in the above and R^{2″} represents C₁₋₆ alkyl which may optionally have been substituted.]

Compound (14a) can be produced from compound (15) or compound (16) via compound (17), according to the pathway described in Scheme 7.

Compound (17) can be produced by the substitution reaction of compound (15) using the base and alcohols. Specifically, the base is used in an amount of about 1.0 to 10.0 mol, preferably about 1.0 to 5.0 mol, and the alcohols is used in an amount of about 1.0 to 100.0 mol, preferably 1.0 to 2.0 mol, relative to 1 mol of compound (15). The base includes basic salts such as sodium carbonate, potassium carbonate and the like, metal hydrides such as sodium hydride, potassium hydride and the like. The alcohol includes ethanol, 2,2,2-trifluoroethanol, cyclopropylmethanol, 2-propanol, 2-methylpropanol, 2,2,3,3,3-pentafluoropropanol and the like. The present reaction is preferably carried out without using any solvent or by using a solvent inert to the reaction. The preferred solvent includes, but not particularly limited to as long as the reaction proceeds, aromatic hydrocarbons such as benzene, toluene and the like, ethers such as tetrahydrofuran, dioxane, 1,2-dimethoxyethane and the like, amides such as N,N-dimethylformamide, N,N-dimethylacetamide and the like, sulfoxides such as dimethyl sulfoxide and the like, or a mixture solvent thereof. The reaction time is generally 1 hr to 60 hr, preferably 5 hr to 12 hr. The reaction temperature is generally −10 to 200° C., preferably 0 to 150° C.

In addition, compound (17) can be also produced according by the O-alkylation of compound (16) using the base and alkylating agent. Specifically, the base is used in an amount of about 1.0 to 5.0 mol, preferably about 1.0 to 2.0 mol, and the alkylating agent is used in an amount of about 1.0 to 10.0 mol, preferably about 1.0 to 3.0 mol, relative to 1 mol of compound (16). The base includes inorganic bases such as sodium hydroxide, potassium hydroxide, barium hydroxide and the like, basic salts such as sodium carbonate, potassium carbonate, cesium carbonate and the like, metal alkoxides such as sodium methoxide, sodium ethoxide, potassium tert-butoxide and the like, metal hydrides such as sodium hydride, potassium hydride and the like, and organic bases such as triethylamine, imidazole, formamidine and the like. The alkylating agent, various halogenated alkyls such as alkyl chloride, alkyl bromide, alkyl iodide and the like and derivatives thereof, sulfonic acid esters such as p-toluenesulfonic acid ester, methanesulfonic acid ester and the like, and sulfuric acid esters such as dimethyl sulfate and the like. The present reaction is preferably carried out by using a solvent inert to the reaction. The preferred solvent includes, but not particularly limited to as long as the reaction proceeds, aromatic hydrocarbons such as benzene, toluene and the like, ethers such as tetrahydrofuran, dioxane, 1,2-dimethoxyethane and the like, amides such as N,N-dimethylformamide, N,N-dimethylacetamide and the like, sulfoxides such as dimethyl sulfoxide and the like, or a mixture solvent thereof. The reaction time is generally 1 hr to 60 hr, preferably 5 hr to 24 hr. The reaction temperature is generally −10 to 200° C., preferably 0 to 150° C. The resulting compound (17) may be used for the next step in the state of a reaction solution directly or a crude product. Alternatively, according to a typical process, compound (17) can be isolated from the reaction mixture and also can be easily purified by a separation means such as washing, recrystallization, distillation, chromatography and the like.

Compound (14a) can be synthesized according to the reduction reaction of compound (17). Specifically, it is produced under hydrogen atmosphere by using the metal catalyst in an amount of about 0.01 to 5.0 mol, preferably about 0.01 to 2.0 mol, relative to 1 mol of compound (17). The metal catalyst includes palladium-active carbon, palladium hydroxide-active carbon, platinum oxide, platinum and the like. The present reaction is preferably carried out by using a solvent inert to the reaction. The preferred solvent includes, but not particularly limited to as long as the reaction proceeds, alcohols such as methanol, ethanol, propanol, and the like, aromatic hydrocarbons such as benzene, toluene and the like, saturated hydrocarbons such as cyclohexane, hexane and the like, ethers such as tetrahydrofuran, dioxane, 1,2-dimethoxyethane and the like, amides such as N,N-dimethylformamide, N,N-dimethylacetamide and the like, sulfoxides such as dimethyl sulfoxide and the like, water or a mixture solvent thereof. The reaction time is generally 1 hr to 60 hr, preferably 5 hr to 36 hr. The reaction temperature is generally −10 to 200° C., preferably 0 to 150° C. The pressure is about 1 to 10 atm, preferably about 1 to 5 atm. In addition, as another reduction method, the reaction can be carried out using a reducing metal. Specifically, the reducing metal is used in an amount of about 5.0 to 20.0 mol, preferably about 5.0 to 10.0 mol, relative to 1 mol of compound (17). The reducing metal includes reduced iron, tin, zinc and the like. For the purpose of promoting the reaction, hydrochloric acid or salts such as ammonium chloride, calcium chloride and the like can be added. The present reaction is preferably carried out by using a solvent inert to the reaction. The preferred solvent includes, but not particularly limited to as long as the reaction proceeds, alcohols such as methanol, ethanol, propanol and the like, aromatic hydrocarbons such as benzene, toluene and the like, saturated hydrocarbons such as cyclohexane, hexane and the like, ethers such as tetrahydrofuran, dioxane, 1,2-dimethoxyethane and the like, amides such as N,N-dimethylformamide, N,N-dimethylacetamide and the like, ketones such as acetone, methyl ethyl ketone and the like, sulfoxides such as dimethyl sulfoxide and the like, ammonia solution, water or a mixture solvent thereof. The reaction time is generally 1 hr to 60 hr, preferably 5 hr to 36 hr. The reaction temperature is generally −10 to 200° C., preferably 0 to 150° C. The resulting compound (14a) may be used for the next step in the state of a reaction solution directly or a crude product. Alternatively, according to a typical process, compound (14a) can be isolated from the reaction mixture and in particular can be easily purified with a separation means such as washing, recrystallization, distillation, chromatography and the like.

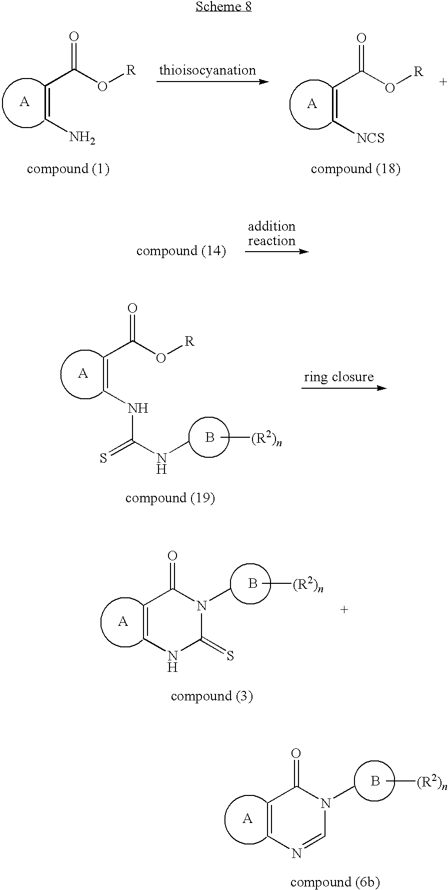

[wherein, each symbol has the same meaning as defined in the above.]

Compound (3) can be produced according to the pathway described in Scheme 8 as another method. Specifically, it can be produced from compound (1) via compound (18), by the ring closure reaction of compound (19). In this case, compound (6b) is may be obtained (provided that, n≧2 except the case in which R² is hydrogen).

Compound (18) can be produced by thioisocyanation of compound (1). Specifically, the thioisocyanating agent is used in an amount of about 1.0 to 5.0 mol, preferably about 1.0 to 2.0 mol, relative to 1 mol of compound (1). The thioisocyanating agent includes thiophosgene, 1,1′-carbonothioyldipyridin-2(1H)-one, di-2-pyridyl thionocarbonate, 1,1′-thiocarbonyl diimidazole and the like. When thiophosgene is used for the present reaction, the reaction can be carried out in the presence of a deacidifying agent to remove the released halogenated hydrogens from the reaction system. For example, the deacidifying agent is preferably added, including basic salts such as sodium carbonate, potassium carbonate, sodium hydrogen carbonate and the like, aromatic amines such as pyridine, lutidine and the like, tertiary amines such as triethylamine, tripropylamine, tributylamine, cyclohexyldimethylamine, 4-dimethylaminopyridine, N,N-dimethylaniline, N-methylpiperidine, N-methylpyrrolidine, N-methylmorpholine and the like. The present reaction is preferably carried out by using a solvent inert to the reaction. The preferred solvent includes, but not particularly limited to as long as the reaction proceeds, halogenated hydrocarbons such as dichloromethane, chloroform, carbon tetrachloride, 1,2-dichloroethane and the like, alcohols such as methanol, ethanol, propanol and the like, aromatic hydrocarbons such as benzene, toluene and the like, saturated hydrocarbons such as cyclohexane, hexane and the like, ethers such as tetrahydrofuran, dioxane, 1,2-dimethoxyethane and the like, amides such as N,N-dimethylformamide, N,N-dimethylacetamide and the like, nitriles such as acetonitrile, propionitrile and the like, sulfoxides such as dimethyl sulfoxide and the like, water or a mixture solvent thereof. The reaction time is generally 30 min to 60 hr, preferably 1 hr to 24 hr. The reaction temperature is generally −10 to 200° C., preferably 0 to 120° C. The resulting compound (18) may be used for the next step in the state of a reaction solution directly or a crude product. Alternatively, according to a typical process, compound (18) can be isolated from the reaction mixture and in particular can be easily purified by a separation means such as recrystallization, distillation, chromatography and the like.

Compound (19) can be produced by the addition reaction of compound (14) to compound (18). Specifically, for the addition reaction, compound (14) is used in an amount of about 1.0 to 3.0 mol, preferably about 1.0 to 1.5 mol relative to 1 mol of compound (18). The present reaction is preferably carried out by using a solvent inert to the reaction. The preferred solvent includes, but not particularly limited to as long as the reaction proceeds, halogenated hydrocarbons such as dichloromethane, chloroform, carbon tetrachloride, 1,2-dichloroethane and the like, alcohols such as methanol, ethanol, propanol and the like, aromatic hydrocarbons such as benzene, toluene and the like, saturated hydrocarbons such as cyclohexane, hexane and the like, ethers such as tetrahydrofuran, dioxane, 1,2-dimethoxyethane and the like, amides such as N,N-dimethylformamide, N,N-dimethylacetamide and the like, nitriles such as acetonitrile, propionitrile and the like, sulfoxides such as dimethyl sulfoxide and the like, or a mixture solvent thereof. The reaction time is generally 1 hr to 60 hr, preferably 1 hr to 3 hr. The reaction temperature is generally −10 to 200° C., preferably 30 to 150° C. The resulting compound (19) may be used for the next step in the state of a reaction solution directly or a crude product. Alternatively, according to a typical process, compound (19) can be isolated from the reaction mixture and in particular can be easily purified with a separation means such as washing, recrystallization, distillation, chromatography and the like.

Compound (3) can be produced by the ring closure reaction of compound (19) in the presence of the base. Further, compound (6b) can be obtained as a byproduct according to this ring closure reaction (provided that, n≧2 except the case in which R² is hydrogen). Specifically, for the ring closure reaction, the base is used in an amount of about 2.0 to 10.0 mol, preferably about 2.0 to 4.0 mol, relative to 1 mol of compound (19). The base includes inorganic bases such as sodium hydroxide, potassium hydroxide, barium hydroxide and the like, basic salts such as sodium carbonate, potassium carbonate and the like, metal alkoxides such as sodium methoxide, sodium ethoxide, potassium tert-butoxide and the like, and metal hydrides such as sodium hydride, potassium hydride and the like. The present reaction is preferably carried out by using a solvent inert to the reaction. The preferred solvent includes, but not particularly limited to as long as the reaction proceeds, alcohols such as methanol, ethanol, propanol and the like, aromatic hydrocarbons such as benzene, toluene and the like, saturated hydrocarbons such as cyclohexane, hexane and the like, ethers such as tetrahydrofuran, dioxane, 1,2-dimethoxyethane and the like, amides such as N,N-dimethylformamide, N,N-dimethylacetamide and the like, nitriles such as acetonitrile, propionitrile and the like, sulfoxides such as dimethyl sulfoxide and the like, water, or a mixture solvent thereof. The reaction time is generally 30 min to 12 hr, preferably 30 min to 2 hr. The reaction temperature is generally −10 to 200° C., preferably 30 to 150° C. The resulting compound (3) and compound (6b) can be isolated from the reaction mixture according to a typical process and in particular can be easily purified with a separation means such as washing, recrystallization, distillation, chromatography and the like.

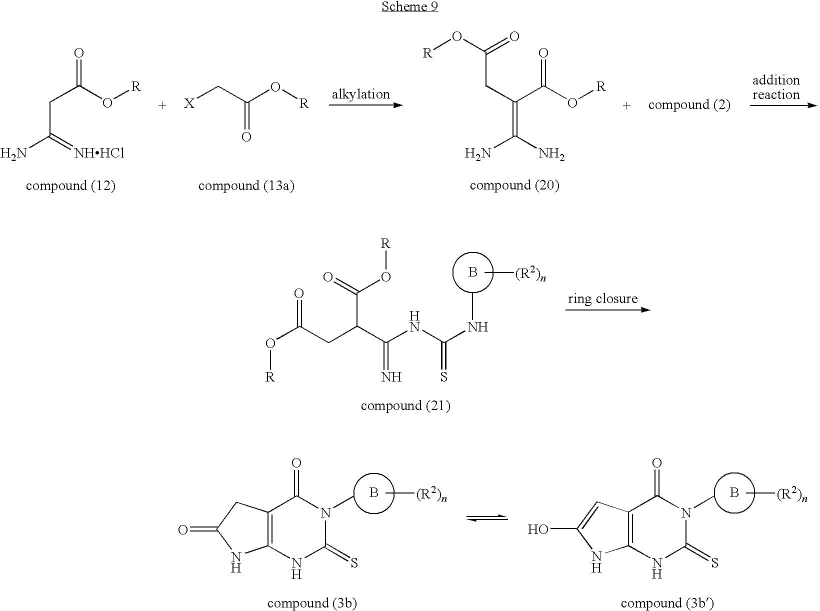

[wherein, each symbol has the same meaning as defined in the above.]

Compounds (3b) and (3b′), which is the tautomer of compound (3b), can be produced by the ring closure reaction of compound (21), which is produced from compound (12) via compound (20), according to the pathway described in Scheme 9.

Compound (20) can be produced by the alkylation reaction of compound (12) using compound (13a) and the base. Specifically, compound (13a) is used for the reaction in an amount of about 1.0 to 3.0 mol, preferably 1.0 to 1.5 mol, relative to 1 mol of compound (12). The base is used in an amount of about 1.0 to 2.0 mol, preferably about 1.0 to 1.5 mol, relative to 1 mol of compound (12). As for compound (13a), methyl chloroacetate, ethyl bromoacetate, isopropyl bromoacetate and the like are used. Further, the base includes basic salts such as sodium hydrogen carbonate, sodium carbonate, potassium carbonate and the like, metal alkoxides such as sodium methoxide, sodium ethoxide, potassium tert-butoxide and the like, metal hydrides such as sodium hydride, potassium hydride and the like, and organic bases such as triethylamine, imidazole, formamidine and the like. The present reaction is preferably carried out by using a solvent inert to the reaction. The preferred solvent includes, but not particularly limited to as long as the reaction proceeds, halogenated hydrocarbons such as dichloromethane, chloroform, carbon tetrachloride, 1,2-dichloroethane and the like, alcohols such as methanol, ethanol, propanol, 1,1-dimethylethanol and the like, aromatic hydrocarbons such as benzene, toluene and the like, ethers such as tetrahydrofuran, dioxane, 1,2-dimethoxyethane and the like, amides such as N,N-dimethylformamide, N,N-dimethylacetamide and the like, sulfoxides such as dimethyl sulfoxide and the like, nitriles such as acetonitrile, propionitrile and the like,water or a mixture solvent thereof. The reaction time is generally 30 min to 12 hr, preferably 45 min to 2 hr. The reaction temperature is generally −10 to 200° C., preferably 0 to 40° C. The resulting compound (20) may be used for the next step in the state of a reaction solution directly or a crude product. Alternatively, according to a typical process, compound (20) can be isolated from the reaction mixture and in particular can be purified with a separation means such as washing, recrystallization, distillation, chromatography and the like.

Compound (21) can be produced by the addition reaction of compound (20) to compound (2). Specifically, the compound (2) is used for the addition reaction in an amount of about 0.3 to 2.0 mol, preferably about 0.3 to 1.5 mol, relative to 1 mol of compound (20). The present reaction is preferably carried out by using a solvent inert to the reaction. The preferred solvent includes, but not particularly limited to as long as the reaction proceeds, halogenated hydrocarbons such as dichloromethane, chloroform, carbon tetrachloride, 1,2-dichloroethane and the like, alcohols such as methanol, ethanol, propanol, 1,1-dimethylethanol and the like, aromatic hydrocarbons such as benzene, toluene and the like, ethers such as tetrahydrofuran, dioxane, 1,2-dimethoxyethane and the like, amides such as N,N-dimethylformamide, N,N-dimethylacetamide and the like, sulfoxides such as dimethyl sulfoxide and the like, nitriles such as acetonitrile, propionitrile and the like, water, or a mixture solvent thereof The reaction time is generally 1 hr to 12 hr, preferably 45 min to 2 hr. The reaction temperature is generally −10 to 200° C., preferably 30 to 150° C. The resulting compound (21) may be used for the next step in the state of a reaction solution directly or a crude product. Alternatively, according to a typical process, compound (21) can be isolated from the reaction mixture and in particular can be purified with a separation means such as washing, recrystallization, distillation, chromatography and the like. Compound (21) may be as a mixture of its tautomers or as a single compound of either of its tautomers.

Compounds (3b) and (3b′), which is the tautomer of compound (3b), can be produced by the ring closure reaction of compound (21) using the base. Specifically, the ring closure reaction is carried out by using the base in an amount of about 1.0 to 10.0 mol, preferably 1.0 to 5.0 mol, relative to 1 mol of compound (21). The present reaction is preferably carried out by using a solvent inert to the reaction. The preferred solvent includes, but not particularly limited to as long as the reaction proceeds, alcohols such as methanol, ethanol, propanol, 1,1-dimethylethanol and the like, aromatic hydrocarbons such as benzene, toluene and the like, ethers such as tetrahydrofuran, dioxane, 1,2-dimethoxyethane and the like, amides such as N,N-dimethylformamide, N,N-dimethylacetamide and the like, sulfoxides such as dimethyl sulfoxide and the like, nitriles such as acetonitrile, propionitrile and the like, water, or a mixture solvent thereof. The reaction time is generally 15 min to 12 hr, preferably 15 min to 2 hr. The reaction temperature is generally −10 to 200° C., preferably 0 to 60° C. A reaction product is obtained as a single compound of either compound (3b) or compound (3b′), which is the tautomer of compound (3b), or as their mixture, and it may be used for the next step in the state of a reaction solution directly or a crude product. Alternatively, according to a typical process, a single compound of either compound (3b) or compound (3b′), or a their mixture, can be isolated from the reaction mixture and in particular can be purified with a separation means such as washing, recrystallization, distillation, chromatography and the like.

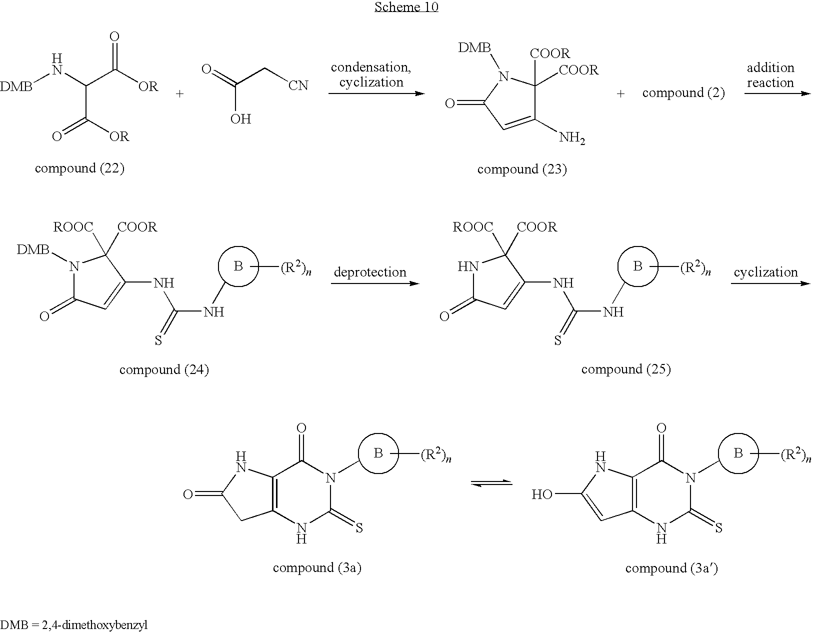

[wherein, each symbol has the same meaning as defined in the above.]

Compound (3a) and compound (3a′), which is the tautomer of compound (3a), can be produced according to the pathway described in Scheme 10. Specifically, it can be produced by the ring closure reaction of compound (25), which is produced from compound (22) via compound (23) and compound (24).

Compound (23) can be produced by condensation reaction between compound (22) and α-cyanoacetic acid followed by cyclization reaction. Specifically, the reaction is carried out by using about 1.0 to 5.0 mol, preferably about 1.0 to 1.5 mol of α-cyanoacetic acid relative to 1 mol of compound (22) in the presence of an appropriate condensing agent. The condensing agent includes N,N′-disubstituted carbodiimides such as N,N′-dicyclohexylcarbodiimide, 1-ethyl-3-(3-dimethylaminopropyl)carbodiimide (WSC) hydrochloride and the like, azolides such as N,N′-carbonyldiimidazole and the like, a dehydrating agent such as N-ethoxycarbonyl-2-ethoxy-1,2-dihydroquinoline, oxyphosphorous chloride, alkoxyacetylene and the like, 2-halogenopyridinium salt such as 2-chloromethylpyridinium iodide, 2-fluoro-l-methylpyridinium iodide and the like, and it is used in an amount of about 1.0 to 5.0 mol, preferably about 1.0 to 2.0 mol, relative to 1 mol of compound (22). In addition, instead of the carboxylic acids, their salts and reactive derivatives can be also used. The reactive derivatives of carboxylic acids are used, including, for example, acid halides (e.g., acid chloride, acid bromide and the like), acid amides (e.g., acid amides with pyrazole, imidazole, benzotriazole and the like), acid anhydrides, acid azides, active esters (e.g., diethoxyphosphoric acid ester, diphenoxyphosphoric acid ester, p-nitrophenyl ester, 2,4-dinitrophenyl ester, cyanomethyl ester, pentachlorophenyl ester, esters with N-hydroxysuccinimide, esters with N-hydroxyphthalimide, esters with 1-hydroxybenzotriazole, esters with 6-chloro-1-hydroxybenzotriazole, esters with 1-hydroxy-1H-2-pyridone and the like), active thioester (e.g., 2-pyridylthio ester, 2-benzothiazolyl thioester and the like) and the like. The present reaction is preferably carried out by using a solvent inert to the reaction. The preferred solvent includes, but not particularly limited to as long as the reaction proceeds, halogenated hydrocarbons such as dichloromethane, chloroform, carbon tetrachloride, 1,2-dichloroethane and the like, aromatic hydrocarbons such as benzene, toluene and the like, ethers such as tetrahydrofuran, dioxane, 1,2-dimethoxyethane and the like, amides such as N,N-dimethylformamide, N,N-dimethylacetamide and the like, sulfoxides such as dimethyl sulfoxide and the like, or a mixture solvent thereof. The reaction time is generally 1 hr to 24 hr, preferably 1 hr to 15 hr. The reaction temperature is generally −10 to 200° C., preferably 0 to 60° C. The resulting compound (23) may be used for the next step in the state of a reaction solution directly or a crude product. Alternatively, according to a typical process, compound (23) can be isolated from the reaction mixture and in particular can be purified with a separation means such as washing, recrystallization, distillation, chromatography and the like. In addition, compound (23) can be produced according to a known method, for example, by a method described in Tetrahedron, vol. 41, page 479 (1985) and the like, or a method pursuant thereto.

Compound (24) can be produced by the addition reaction of compound (23) to compound (2) in the presence of the base. Specifically, compound (2) is used in an amount of about 1.0 to 2.0, preferably about 1.0 to 1.3 mol, relative to 1 mol of compound (23), and the base is used in an amount of about 1.0 to 2.0, preferably about 1.0 to 1.2 mol, relative to 1 mol of compound (23) for the addition reaction. The base includes basic salts such as sodium hydrogen carbonate, sodium carbonate, potassium carbonate and the like, metal alkoxides such as sodium methoxide, sodium ethoxide, potassium tert-butoxide and the like, and metal hydrides such as sodium hydride, potassium hydride and the like. The present reaction is preferably carried out by using a solvent inert to the reaction. The preferred solvent includes, but not particularly limited to as long as the reaction proceeds, aromatic hydrocarbons such as benzene, toluene and the like, ethers such as tetrahydrofuran, dioxane, 1,2-dimethoxyethane and the like, amides such as N,N-dimethylformamide, N,N-dimethylacetamide and the like, sulfoxides such as dimethyl sulfoxide and the like, or a mixture solvent thereof. The reaction time is generally 30 min to 24 hr, preferably 30 min to 3 hr. The reaction temperature is generally −10 to 200° C., preferably 0 to 40° C. The resulting compound (24) may be used for the next step in the state of a reaction solution directly or a crude product. Alternatively, according to a typical process, compound (24) can be isolated from the reaction mixture and in particular can be purified with a separation means such as washing, recrystallization, distillation, chromatography and the like.

Compound (25) can be produced from compound (24) by deprotection under acidic condition. For the deprotection of a protecting group of compound (24) (i.e., 2,4-dimethoxybenzyl group), mineral acids such as hydrochloric acid, sulfuric acid and the like, Lewis acids such as boron trichloride, boron tribromide and the like, combined use of Lewis acid and thiol or sulfide, organic acids such as trifluoroacetic acid, p-toluenesulfonic acid and the like, combined use of the organic acids and anisole, and the like are generally effective. Specifically, the acidic compound is used in an amount of about 0.5 to 20.0 mol, preferably about 0.5 to 10.0 mol, relative to 1.0 mol of compound (24). The present reaction is preferably carried out without any solvent or by using a solvent which is inert to the reaction. The preferred solvent includes, but not particularly limited to as long as the reaction proceeds, halogenated hydrocarbons such as dichloromethane, chloroform, carbon tetrachloride, 1,2-dichloroethane and the like, alcohols such as methanol, ethanol, propanol and the like, aromatic hydrocarbons such as benzene, toluene and the like, saturated hydrocarbons such as cyclohexane, hexane and the like, organic acids such as formic acid, acetic acid and the like, ethers such as tetrahydrofuran, dioxane, 1,2-dimethoxyethane and the like, amides such as N,N-dimethylformamide, N,N-dimethylacetamide and the like, nitriles such as acetonitrile, propionitrile and the like, ketones such as acetone, methyl ethyl ketone and the like, sulfoxides such as dimethyl sulfoxide and the like, or a mixture solvent thereof. The reaction time is generally 2 hr to 60 hr, preferably 4 hr to 15 hr. The reaction temperature is generally −10 to 200° C., preferably 0 to 60° C. The resulting compound (25) may be used for the next step in the state of a reaction solution directly or a crude product. Alternatively, according to a typical process, compound (25) can be isolated from the reaction mixture and in particular can be easily purified with a separation means such as recrystallization, distillation, chromatography and the like.

Compounds (3a) and (3a′), which is the tautomer of compound (3a), can be produced by the ring closure reaction of compound (25) under basic condition. Specifically, for the ring closure reaction, the base is used in an amount of about 1.0 to 5.0 mol, preferably about 2.0 to 3.0 mol, relative to 1 mol of compound (25). The base includes inorganic bases such as sodium hydroxide, potassium hydroxide, barium hydroxide and the like, basic salts such as sodium carbonate, potassium carbonate and the like, metal alkoxides such as sodium methoxide, sodium ethoxide, potassium tert-butoxide and the like, and metal hydrides such as sodium hydride, potassium hydride and the like. The reaction is preferably carried out by using a solvent which is inert to the reaction. The solvent includes, but not particularly limited to as long as the reaction proceeds, alcohols such as methanol, ethanol, propanol, 1,1-dimethylethanol and the like, aromatic hydrocarbons such as benzene, toluene and the like, ethers such as tetrahydrofuran, dioxane, 1,2-dimethoxyethane and the like, nitriles such as acetonitrile, propionitrile and the like, amides such as N,N-dimethylformamide, N,N-dimethylacetamide and the like, sulfoxides such as dimethyl sulfoxide and the like, water, or a mixture solvent thereof The reaction time is generally 30 min to 12 hr, preferably 30 min to 3 hr. The reaction temperature is generally −10 to 200° C., preferably 0 to 60° C. A reaction product is as a single compound of either compound (3a) or compound (3a′), which is the tautomer of compound (3a), or as their mixture, and it may be used for the next step in the state of a reaction solution directly or a crude product. Alternatively, according to a typical process, a single compound of either compound (3a) or compound (3a′), or a their mixture can be isolated from the reaction mixture and in particular can be purified with a separation means such as washing, recrystallization, distillation, chromatography and the like.

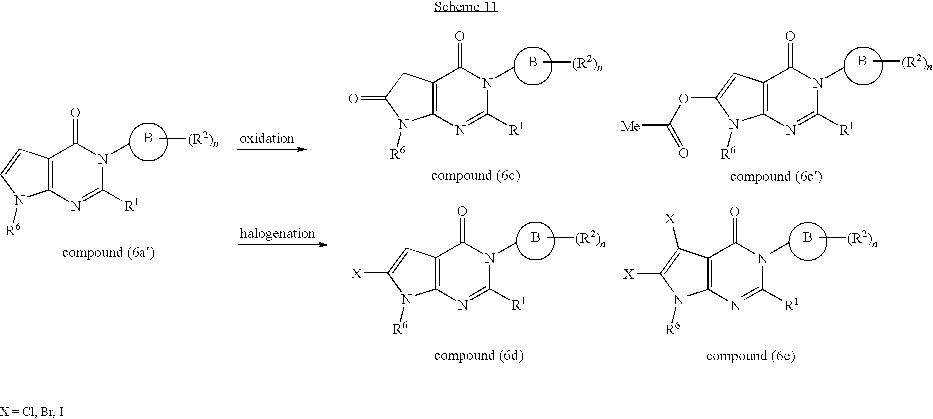

[wherein, each symbol has the same meaning as defined in the above.]

Compound (6c), compound (6d) and compound (6e) can be also produced from compound (6a′) according to the pathway described in Scheme 11.

Compound (6c) can be produced by oxidation of compound (6a′). Specifically, for the oxidation reaction, the oxidizing agent can be used in an amount of about 1.0 to 3.0 mol, preferably about 1.0 to 2.0 mol, relative to 1 mol of compound (6a′). As for the oxidizing agent, a halogen element such as bromine, iodine and the like, pyridiniumbromide perbromide, iodosobenzene diacetate and the like are used. The reaction is preferably carried out by using a solvent which is inert to the reaction. The preferred solvent includes, but not particularly limited to as long as the reaction proceeds, halogenated hydrocarbons such as dichloromethane, chloroform, carbon tetrachloride, 1,2-dichloroethane and the like, alcohols such as methanol, ethanol, propanol, 1,1-dimethylethanol and the like, organic acids such as formic acid, acetic acid and the like, ethers such as tetrahydrofuran, dioxane, 1,2-dimethoxyethane and the like, amides such as N,N-dimethylformamide, N,N-dimethylacetamide and the like, sulfoxides such as dimethyl sulfoxide and the like, water or a mixture solvent thereof. The reaction time is generally 30 min to 60 hr, preferably 30 min to 12 hr. The reaction temperature is generally −10 to 200° C., preferably 0 to 120° C. The compound (6c) can be isolated from the reaction mixture according to a typical process and in particular can be easily purified with a separation means such as recrystallization, distillation, chromatography and the like. In the case of production of compound (6c) using iodosobenzene diacetate as the oxidizing agent, compound (6c′) is also obtained.

Compound (6d) and compound (6e) can be produced by halogenation of compound (6a′). Specifically, for the halogenation reaction, the halogenating agent is used in an amount of about 1.0 to 5.0 mol, preferably about 1.0 to 3.0 mol, relative to 1 mol of compound (6a′). The halogenating agent includes a halogen element such as chlorine, bromine, iodine and the like, N-halogenated imides such as N-chlorosuccinimide, N-bromosuccinimide, N-iodosuccinimide, N-chlorophthalimide, N-bromophthalimide and the like. The reaction is preferably carried out by using a solvent which is inert to the reaction. The preferred solvent includes, but not particularly limited to as long as the reaction proceeds, halogenated hydrocarbons such as dichloromethane, chloroform, carbon tetrachloride, 1,2-dichloroethane and the like, alcohols such as methanol, ethanol, propanol, 1,1-dimethylethanol and the like, organic acids such as formic acid, acetic acid and the like, ethers such as tetrahydrofuran, dioxane, 1,2-dimethoxyethane and the like, amides such as N,N-dimethylformamide, N,N-dimethylacetamide and the like, sulfoxides such as dimethyl sulfoxide and the like, or a mixture solvent thereof. The reaction time is generally 30 min to 60 hr, preferably 30 min to 12 hr. The reaction temperature is generally −10 to 200° C., preferably 0 to 100° C. A reaction product is obtained as a single compound of either compound (6d) or compound (6e), or as their mixture. According to a typical process, each can be isolated from the reaction mixture and in particular can be easily purified with a separation means such as recrystallization, distillation, chromatography and the like.

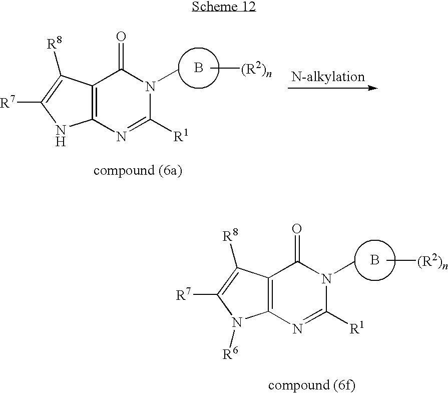

[wherein, each symbol has the same meaning as defined in the above.]

When R⁶ is C₁₋₆ alkyl which may have a substituent group or C₃₋₈ cycloalkyl which may have a substituent group, compound (6f) can also be produced from compound (6a) by N-alkyration using the base (Scheme 12). Specifically, the base is used for the alkylation in an amount of about 1.0 to 3.0, preferably 1.0 to 2.0 mol, relative to 1 mol of compound (6a). The alkylating agent is used in an amount of about 1.0 to 20.0 mol, preferably about 1.0 to 10.0 mol, relative to 1 mol of compound (6a). The base includes inorganic bases such as sodium hydroxide, potassium hydroxide, barium hydroxide and the like, basic salts such as sodium carbonate, potassium carbonate and the like, and metal alkoxides such as sodium methoxide, sodium ethoxide, potassium tert-butoxide and the like, metal hydrides such as sodium hydride, potassium hydride and the like. The alkylating agent includes various halogenated alkyls such as methyl iodide, ethyl iodide, propyl iodide and the like, sulfuric acid esters such as dimethyl sulfate, diethyl sulfate and the like, sulfonic acid esters such as p-toluenesulfonic acid methyl ester, methanesulfonic acid methyl ester and the like. The present reaction is preferably carried out by using a solvent which is inert to the reaction. The preferred solvent includes, but not particularly limited to as long as the reaction proceeds, alcohols such as methanol, ethanol, propanol, 1,1-dimethylethanol and the like, aromatic hydrocarbons such as benzene, toluene and the like, ethers such as tetrahydrofuran, dioxane, 1,2-dimethoxyethane and the like, amides such as N,N-dimethylformamide, N,N-dimethylacetamide and the like, sulfoxides such as dimethyl sulfoxide and the like, or a mixture solvent thereof. The reaction time is generally 30 min to 60 hr, preferably 30 min to 24 hr. The reaction temperature is generally −10 to 200° C., preferably 0 to 150° C. The compound (60 can be isolated from the reaction mixture according to a typical process and in particular can be easily purified with a separation means such as washing, recrystallization, distillation, chromatography and the like.

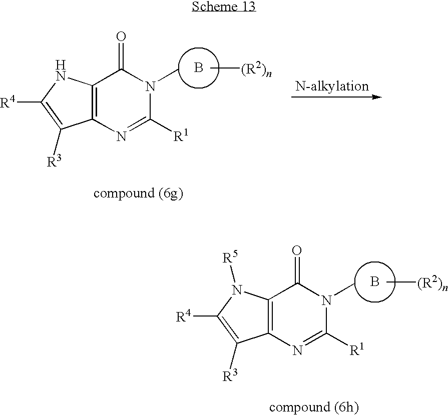

[wherein, each symbol has the same meaning as defined in the above.]

When R⁵ is C₁₋₆ alkyl which may have a substituent group, compound (6h) can also be produced from compound (6g) by N-alkylation using the base (Scheme 13). Specifically, the base is used for the alkylation in an amount of about 1.0 to 3.0, preferably 1.0 to 2.0 mol, relative to 1 mol of compound (6g). The alkylating agent is used in an amount of about 1.0 to 20.0 mol, preferably about 1.0 to 10.0 mol, relative to 1 mol of compound (6g). The base includes inorganic bases such as sodium hydroxide, potassium hydroxide, barium hydroxide and the like, basic salts such as sodium carbonate, potassium carbonate and the like, and metal alkoxides such as sodium methoxide, sodium ethoxide, potassium tert-butoxide and the like, and metal hydrides such as sodium hydride, potassium hydride and the like. The alkylating agent includes various halogenated alkyls such as methyl iodide, ethyl iodide, propyl iodide and the like, sulfuric acid esters such as dimethyl sulfate, diethyl sulfate and the like, sulfonic acid esters such as p-toluenesulfonic acid methyl ester, methanesulfonic acid methyl ester and the like. The present reaction is preferably carried out by using a solvent which is inert to the reaction. The preferred solvent includes, but not particularly limited to as long as the reaction proceeds, alcohols such as methanol, ethanol, propanol, 1,1-dimethylethanol and the like, aromatic hydrocarbons such as benzene, toluene and the like, ethers such as tetrahydrofuran, dioxane, 1,2-dimethoxyethane and the like, amides such as N,N-dimethylformamide, N,N-dimethylacetamide and the like, sulfoxides such as dimethyl sulfoxide and the like, or a mixture solvent thereof. The reaction time is generally 30 min to 60 hr, preferably 30 min to 24 hr. The reaction temperature is generally −10 to 200° C., preferably 0 to 150° C. The compound (6h) can be isolated from the reaction mixture according to a typical process and in particular can be easily purified with a separation means such as washing, recrystallization, distillation, chromatography and the like.

Further, compound (6i), in which R^{1a} is C₁₋₆ alkyl which may have a substituent group or C₃₋₈ cycloalkyl which may have a substituent group, can be produced according to the pathway described in Scheme 14, for example.

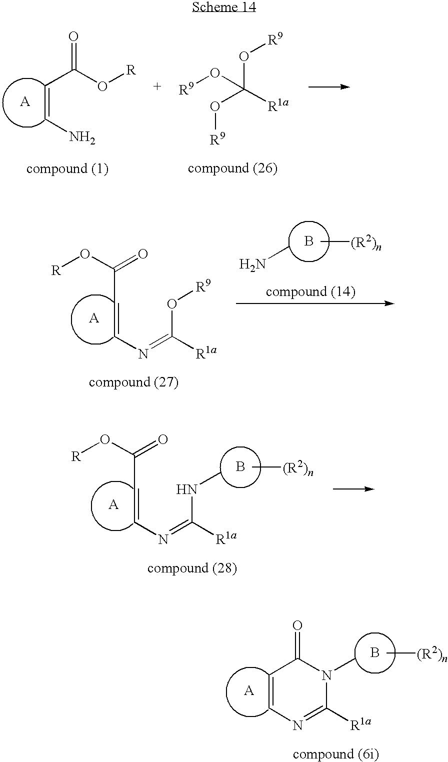

[wherein, R^{1a} is C₁₋₆ alkyl which may have a substituent group or C₃₋₈ cycloalkyl which may have a substituent group, R⁹ is C₁₋₄ alkyl, and R, R² and n have the same meanings as defined in the above.]

Compound (6i) can be produced by the ring closure reaction of compound (28), which is obtained by the substitution reaction of compound (14) to compound (27) produced from compound (1) and compound (26).

Production of compound (27) from compound (1) and compound (26) can be carried out without using any solvent. Specifically, compound (26) is used in an amount of 1 to 100 mol, preferably 1 to 50 mol, relative to 1 mol of compound (1). The reaction time is generally 1 hr to 60 hr, preferably 1 hr to 24 hr. The reaction temperature is generally 0 to 200° C., preferably 50 to 150° C. In addition, the present reaction can be carried out by using a solvent which is inert to the reaction. The preferred solvent includes, but not particularly limited to as long as the reaction proceeds, aromatic hydrocarbons such as benzene, toluene and the like, ethers such as tetrahydrofuran, dioxane, 1,2-dimethoxyethane and the like, amides such as N,N-dimethylformamide, N,N-dimethylacetamide and the like, sulfoxides such as dimethyl sulfoxide and the like, or a mixture solvent thereof The compound (27) can be isolated from the reaction mixture according to a typical process and in particular can be easily purified with a separation means such as washing, recrystallization, distillation, chromatography and the like.

Production of compound (28) from compound (27) can be carried out by the substitution reaction of compound (14) to compound (27). Specifically, compound (14) is used in an amount of 1 to 5 mol, preferably 1 to 2 mol, relative to 1 mol of compound (27). The reaction time is generally 1 hr to 60 hr, preferably 1 hr to 24 hr. The reaction temperature is generally 0 to 200° C., preferably 50 to 150° C. In addition, the present reaction is preferably carried out by using a solvent which is inert to the reaction. The preferred solvent includes, but not particularly limited as long as the reaction proceeds, aromatic hydrocarbons such as benzene, toluene and the like, ethers such as tetrahydrofuran, dioxane, 1,2-dimethoxyethane and the like, amides such as N,N-dimethylformamide, N,N-dimethylacetamide and the like, sulfoxides such as dimethyl sulfoxide and the like, or a mixture solvent thereof. Compound (28) can be isolated from the reaction mixture according to a typical process and in particular can be easily purified with a separation means such as washing, recrystallization, distillation, chromatography and the like.

Compound (6i) can be produced by the ring closure reaction of compound (28). Specifically, the reaction can be carried out by heating compound (28) in an appropriate solvent. In the present reaction, it is preferred to use dealcoholating agent such as phosphorus pentoxide and the like. The reaction time is generally 1 hr to 60 hr, preferably 1 hr to 24 hr. The reaction temperature is generally 0 to 200° C., preferably 50 to 150° C. In addition, the present reaction is preferably carried out by using a solvent which is inert to the reaction. The preferred solvent includes, but not particularly limited as long as the reaction proceeds, aromatic hydrocarbons such as benzene, toluene and the like, ethers such as tetrahydrofuran, dioxane, 1,2-dimethoxyethane and the like, amides such as N,N-dimethylformamide, N,N-dimethylacetamide and the like, sulfoxides such as dimethyl sulfoxide and the like, or a mixture solvent thereof Compound (6i) can be isolated from the reaction mixture according to a typical process and in particular can be easily purified with a separation means such as washing, recrystallization, distillation, chromatography and the like.

In each of the above-described reactions, when the raw compound has an amino (including —NH— and —NH₂—), carboxyl, hydroxy, carbonyl or mercapto group, a protective group conventionally used in peptide chemistry or the like may be introduced into these groups. The compound of interest may be obtained by removing such a protective group after reaction, if necessary.

Examples of amino protective groups include, but are not limited to, formyl group, C₁₋₆ alkyl-carbonyl groups, C₁₋₆ alkoxy-carbonyl groups, benzoyl group, C₇₋₁₀ aralkyl-carbonyl groups (e.g., benzylcarbonyl), C₇₋₁₄ aralkyloxy-carbonyl groups (e.g., benzyloxycarbonyl, 9-fluorenylmethoxycarbonyl), trityl group, phthaloyl group, N,N-dimethylaminomethylen groups, substituted silyl groups (e.g., trimethylsilyl, triethylsilyl, dimethylphenylsilyl, tert-butyldimethylsilyl, tert-butyldiethylsilyl), C₂₋₆ alkenyl groups (e.g., 1-allyl), substituted C₇₋₁₀ aralkyl groups (e.g., 2,4-dimethoxybenzyl) and the like. These groups may be substituted with one to three substituents selected from halogen atoms, C₁₋₆ alkoxy groups and nitro group.

Examples of carboxyl protective groups include, but are not limited to, C₁₋₆ alkyl groups, C₇₋₁₁ aralkyl groups (e.g., benzyl), phenyl group, trityl group, substituted silyl groups (e.g., trimethylsilyl, triethylsilyl, dimethylphenylsilyl, tert-butyldimethylsilyl, tert-butyldiethylsilyl) and C₂₋₆ alkenyl groups (e.g., 1-allyl). These groups may be substituted with one to three substituents selected from halogen atoms, C₁₋₆ alkoxy groups and nitro group.

Examples of hydroxy protective groups include, but are not limited to, C₁₋₆ alkyl groups, phenyl group, trityl group, C₇₋₁₀ aralkyl groups (e.g., benzyl), formyl group, C₁₋₆ alkyl-carbonyl groups, benzoyl group, C₇₋₁₀ aralkyl-carbonyl groups (e.g., benzylcarbonyl), 2-tetrahydropyranyl group, 2-tetrahydrofuranyl group, substituted silyl groups (e.g., trimethylsilyl, triethylsilyl, dimethylphenylsilyl, tert-butyldimethylsilyl, tert-butyldiethylsilyl) and C₂₋₆ alkenyl groups (e.g., 1-allyl). These groups may be substituted with one to three substituents selected from halogen atoms, C₁₋₆ alkyl groups, C₁₋₆ alkoxy groups and nitro group.

Examples of carbonyl protective groups include, but are not limited to, cyclic acetal groups (e.g., 1,3-dioxane) and non-cyclic acetal groups (e.g., di-C₁₋₆ alkylacetal).

Example of mercapto protective groups include, but are not limited to, C₁₋₆ alkyl groups, phenyl group, trityl group, C₇₋₁₀ aralkyl groups (e.g., benzyl), C₁₋₆ alkyl-carbonyl groups, benzoyl group, C₇₋₁₀ aralkyl-carbonyl groups (e.g., benzylcarbonyl), C₁₋₆ alkoxy-carbonyl groups, C₆₋₁₄ aryloxy-carbonyl groups (e.g., phenyloxycarbonyl), C₇₋₁₄ aralkyloxy-carbonyl groups (e.g., benzyloxycarbonyl, 9-fluorenylmethoxycarbonyl), 2-tetrahydropyranyl group and C₁₋₆ alkylamino-carbonyl groups (e.g., methylaminocarbonyl, ethylaminocarbonyl). These groups may be substituted with one to three substituents selected from halogen atoms, C₁₋₆ alkyl groups, C₁₋₆ alkoxy groups and nitro group.

Removal of the above-listed protective groups may be performed according to a method known per se (e.g., the method described in Protective Groups in Organic Synthesis published by John Wiley and Sons (1980)). Specifically, methods using acid, base, UV light, hydrazine, phenylhydrazine, sodium N-methyldithiocarbamate, tetrabutylammonium fluoride, palladium acetate, trialkylsilyl halide (e.g., trimethylsilyl iodide, trimethylsilyl bromide) or the like, reduction methods and so forth may be enumerated.

Hereinafter, the present invention will be described in detail by way of Reference Examples, Examples, Preparation Examples and Test Examples, but the present invention is not intended to be limited thereto.

Elution during column chromatography in the Reference Examples and Examples was carried out under the monitoring with a UV detector or by TLC (Thin Layer Chromatography). In the monitoring by TLC, a Kieselgel 60F₂₅₄ plate manufactured by Merck, Ltd. or a NH (propylaminated) silica gel plate manufactured by Fuji Silisia Chemical, Ltd. were used as TLC plates. As for the column, a silica gel or a NH (propylaminated) silica gel, both manufactured by Fuji Silisya Chemical, Ltd., were used. The NMR spectrum represents a proton NMR, and the measurement was made with a Bruker AVANCE400 (400 MHz type spectrophotometer) or a Bruker AVANCE300 (300 MHz type spectrophotometer), using tetramethylsilane as an internal standard. The chemical shift is expressed as a 5 value, and the coupling constant is expressed in Hz.

The abbreviations used in the Reference Examples and Examples have the following meanings.

s: Singlet

d: Doublet

t: Triplet

q: Quartet

dd: Double doublet

ddd: Double double doublet

dt: Double triplet

td: Triple doublet

tt: Triple triplet

tq: Triple quartet

spt: Septet

sxt: Sextet

br. s.: Broad (broad-ranging) singlet

m: Multiplet

J: Coupling constant

Hz: Hertz

CHLOROFORM-d: Deuterated chloroform

DMSO-d₆: Deuterated dimethylsulfoxide

¹H NMR: Proton nuclear magnetic resonance

In regard to ¹H NMR, no indication is provided for those giving very low peaks with respect to proton, such as a hydroxyl group or an amino group.

In the Reference Examples and Examples described below, HPLC-Mass Spectroscopy (LC-MS) measurement was made under the following conditions.

Measuring instrument: Micromass ZQ-Alliance HT by Waters Corp.

Column: CAPCELL PAK C18UG120, S-3 μm, 1.5×35 mm

Solvent: Liquid A; 0.05% trifluoroacetic acid-containing water, Liquid B; 0.04% trifluoroacetic acid-containing acetonitrile

Gradient cycle: 0.00 minute (liquid A/liquid B=90/10), 2.00 minutes (liquid A/liquid B=5/95), 2.75 minutes (liquid A/liquid B=5/95), 2.76 minutes (liquid A/liquid B=90/10), 3.45 minutes (liquid A/liquid B=90/10)

Injection amount: 2 μl, flow rate: 0.5 ml/min, detection method: UV 220 nm

Ionization method: Electron Spray Ionization (ESI)

### Reference Example 1

### Ethyl 3-amino-5-methyl-1H-pyrrol-2-carboxylate

### Reference Example 1a)

A 20% sodium ethoxide-ethanol solution (34.0 g) was dissolved in ethanol (30 ml), and to this solution, a solution of 5-methylisoxazole (8.3 g) dissolved in ethanol (30 ml) was added dropwise over 10 minutes. The resulting mixture was stirred for one hour at room temperature, and then was stirred for 30 minutes with ice cooling. Then, petroleum ether (30 ml) was added to the reaction mixture liquid. Precipitates were collected by filtration, and were washed with petroleum ether to give a brown solid (8.36 g).

This brown solid (1.05 g) was added to a solution of diethyl aminomalonate hydrochloride (2.12 g) in ethanol (40 ml), and the resulting mixture was stirred overnight at room temperature. Then, the reaction mixture liquid was filtered, and the filtrate was concentrated under reduced pressure to obtain a yellow oily crude product, which was purified by chromatography to give diethyl [(2-cyano-1-methylethenyl)amino]propanedioate (1.59 g) as a yellowish white solid.

¹H NMR (300 MHz, DMSO-d₆) δ ppm 1.21 (6H, t, J=7.1 Hz), 2.05 (3H, s), 3.94 (1H, s), 4.20 (2H, q, J=7.1 Hz), 4.21 (2H, q, J=7.1 Hz), 4.95 (1H, d, J=8.0 Hz), 7.53 (1H, d, J=8.0 Hz).

### Reference Example 1b)

Potassium tert-butoxide (1.65 g) was dissolved in ethanol (30 ml), and to this solution was added a solution of diethyl [(2-cyano-1-methylethenyl)amino]propanedioate (1.5 g) obtained in Reference Example (1a) in ethanol (5 ml) at room temperature. The reaction mixture liquid was heated to reflux for 4 hours, and then ethanol was evaporated under reduced pressure, to obtain a brown oily substance. This oily substance was dissolved in water (20 ml), and the solution was first made into an acidic solution with 1 M hydrochloric acid. Then, sodium hydrogen carbonate was added thereto to return the solution to be weakly alkaline. Subsequently, this aqueous solution was salted out, and was extracted with ethyl acetate. Then, the organic layer was washed with saturated brine, dried over anhydrous magnesium sulfate, and then concentrated under reduced pressure, to obtain an orange-colored crude solid, which was purified by chromatography to give the title compound, ethyl 3-amino-5-methyl-1H-pyrrol-2-carboxylate (747 mg) as a yellowish white solid.

¹H NMR (300 MHz, DMSO-d₆) δ ppm 1.24 (3H, t, J=7.2 Hz), 2.06 (3H, s), 4.14 (2H, q, J=7.2 Hz), 4.93 (2H, br. s.), 5.29 (1H, d, J=2.7 Hz), 10.23 (1H, br. s.).

### Reference Example 2

### Ethyl 3-amino-4-methyl-1H-pyrrol-2-carboxylate

### Reference Example 2a)

2-Methyl-3-oxopropanenitrile (670 mg) obtained by a method described in a published document, Journal of Heterocyclic Chemistry (J. Heterocyclic Chem.), Vol. 21, p. 389 (1984), or a method pursuant to thereto, and diethyl aminomalonate hydrochloride (2.21 g) were dissolved in ethanol (40 ml), and triethylamine (1.24 ml) was added to this solution. The resulting mixture was stirred for 3 days at room temperature. Subsequently, the reaction liquid was concentrated under reduced pressure, to obtain a yellow residue. Ethyl acetate (200 ml) and a saturated aqueous solution of sodium hydrogen carbonate were added to the residue, and the organic layer was collected by partition. Then, the organic layer was washed with saturated brine, dried over anhydrous magnesium sulfate, and then concentrated under reduced pressure, to obtain a yellow oily substance. This oily substance was purified by chromatography, and thus diethyl [(2-cyanoprop-1-en-1-yl)amino]propanedioate (1.11 g) was obtained as a yellow oily substance.

¹H NMR (300 MHz, DMSO-d₆) δ ppm 1.21 (6H, t, J=7.1 Hz), 1.65 (3H, s), 4.19 (2H, q, J=7.1 Hz), 4.18 (2H, q, J=7.1 Hz), 4.97 (1H, d, J=8.7 Hz), 6.82 (1H, d, J=12.5 Hz), 7.11 (1H, dd, J=12.5, 8.7 Hz).

### Reference Example 2b)

Potassium tert-butoxide (659 mg) was dissolved in ethanol (20 ml), and to this solution, diethyl [(2-cyanoprop-1-en-1-yl)amino]propanedioate (1.0 g) obtained in Reference Example (2a) dissolved in ethanol (5 ml) was added at room temperature. The reaction mixture liquid was heated to reflux for 4 hours, and was returned to room temperature, and then acetic acid (0.38 ml) was added thereto. Subsequently, ethanol was distilled off under reduced pressure, to obtain a brown oily substance. This oily substance was dissolved in water (20 ml), and sodium hydrogen carbonate was added to make the solution weakly alkaline. Subsequently, this aqueous solution was salted out, and was extracted with ethyl acetate. The organic layer was washed with saturated brine, dried over anhydrous magnesium sulfate, and then concentrated under reduced pressure, to obtain an orange-colored crude solid. This crude solid was purified by chromatography, and thus the title compound, ethyl 3-amino-4-methyl-1H-pyrrol-2-carboxylate (246 mg), was obtained as a yellowish white solid.

¹NMR (300 MHz, DMSO-d₆) δ ppm 1.25 (3H, t, J=7.0 Hz), 1.83 (3H, s), 4.17 (2H, q, J=7.0 Hz), 4.76 (2H, br. s.), 6.52 (1H, d, J=3.2 Hz), 10.27 (1H, br. s.).

### Reference Example 3

### Ethyl 3-amino-4-ethyl-1H-pyrrol-2-carboxylate

### Reference Example 3a)

2-Formylbutanenitrile (1.0 g) obtained by a method described in a published document, Journal of Medicinal Chemistry (J. Med. Chem.), Vol. 25, p. 235 (1982), or a method pursuant to thereto, and diethyl aminomalonate hydrochloride (2.40 g) were dissolved in ethanol (40 ml), and triethylamine (1.24 ml) was added to this solution. The resulting mixture was stirred for one hour at room temperature, and then was heated to reflux for one hour. The reaction mixture was returned to room temperature, and then was concentrated under reduced pressure, to obtain a yellow solid. Ethyl acetate (100 ml) was added to the solid, and the mixture was filtered, and then the filtrate was concentrated under reduced pressure, to obtain a yellow oily substance. This oily substance was purified by chromatography, and thus diethyl [(2-cyanobut-1-en-1-yl)amino]propanedioate (1.22 g) was obtained as a yellow oily substance.

¹H NMR (300 MHz, DMSO-d₆) δ ppm 0.97 (3H, t, J=7.5 Hz), 1.21 (6H, t, J=7.2 Hz), 2.07 (2H, q, J=7.5 Hz), 4.18 (2H, q, J=7.2 Hz), 4.19 (2H, q, J=7.2 Hz), 4.96 (1H, d, J=8.7 Hz), 6.77 (1H, d, J=12.6 Hz), 7.15 (1H, dd, J=12.6, 8.7 Hz).

### Reference Example 3b)

Diethyl [(2-cyanobut-1-en-1-yl)amino]propanedioate (1.2 g) obtained in Reference Example (3a) was dissolved in ethanol (25 ml), and a 20% sodium ethoxide-ethanol solution (3.37 g) was added to this solution. Then, the mixture was heated to reflux for 2 hours. The reaction mixture was returned to room temperature, and then acetic acid (0.85 g) was added thereto. Then, ethanol was evaporated under reduced pressure, to obtain a brown residue. This residue was dissolved in water (20 ml), and the solution was made weakly alkaline with a saturated aqueous solution of sodium hydrogen carbonate. The solution was then salted out, and was extracted with ethyl acetate. The organic layer was washed with saturated brine, dried over anhydrous magnesium sulfate, and then concentrated under reduced pressure, to obtain a brown crude solid. This crude solid was purified by chromatography, and thus the title compound, ethyl 3-amino-4-ethyl-1H-pyrrol-2-carboxylate (817 mg), was obtained as a yellowish white solid.

¹H NMR (300 MHz, DMSO-d₆) δ ppm 1.07 (3H, t, J=7.6 Hz), 1.25 (3H, t, J=7.2 Hz), 2.26 (2H, q, J=7.6 Hz), 4.17 (2H, q, J=7.2 Hz), 4.77 (2H, br. s.), 6.51 (1H, d, J=3.4 Hz), 10.30 (1H, br. s.).

### Reference Example 4

### Ethyl 3 -amino-4-cyclopropyl-1H-pyrrol-2-carboxylate

### Reference Example 4a)

2-Cyclopropyl-3-oxopropanenitrile (2.0 g) obtained by a method described in a published document, WO 04/22559, or a method pursuant to thereto, and diethyl aminomalonate hydrochloride (2.40 g) were dissolved in ethanol (40 ml), and triethylamine (1.24 ml) was added to this solution. The resulting mixture was stirred for 3 days at room temperature, and then the reaction liquid was concentrated under reduced pressure, to obtain a yellow solid. Ethyl acetate (200 ml) and a saturated aqueous solution of sodium hydrogen carbonate were added to the solid, and the organic layer was collected by partition. Then, the organic layer was washed with saturated brine and filtered, and then the filtrate was concentrated under reduced pressure to obtain a yellow oily substance. This oily substance was purified by chromatography, and thus diethyl [(2-cyano-2-cyclopropylethenyl)amino]propanedioate (2.57 g) was obtained as a yellow oily substance.

¹H NMR (300 MHz, DMSO-d₆) δ ppm 0.32-0.41 (2H, m), 0.71-0.80 (2H, m), 1.21 (6H, t, J=7.2 Hz), 1.43-1.57 (1H, m), 4.20 (2H, q, J=7.2 Hz), 4.19 (2H, q, J=7.2 Hz), 5.00 (1H, d, J=8.7 Hz), 6.89 (1H, dd, J=I2.8, 0.8 Hz), 7.13 (1H, dd, J=12.8, 8.7 Hz).

### Reference Example 4b)

Diethyl [(2-cyano-2-cyclopropylethenyl)amino]propanedioate (2.57 g) obtained in Reference Example (4a) was dissolved in ethanol (50 ml), and a 20% sodium ethoxide-ethanol solution (6.90 g) was added to the solution. Then, the mixture was heated to reflux for 2 hours. The reaction mixture was returned to room temperature, and then acetic acid (1.67 ml) was added thereto. Then, ethanol was distilled off under reduced pressure to obtain a brown residue. This residue was dissolved in water (20 ml), and the solution was made weakly alkaline with a saturated aqueous solution of sodium hydrogen carbonate. The solution was then salted out, and was extracted with ethyl acetate. The organic layer was washed with saturated brine, dried over anhydrous magnesium sulfate, and then concentrated under reduced pressure to obtain a brown crude solid. This crude solid was purified by chromatography, and thus the title compound, ethyl 3-amino-4-cyclopropyl-1H-pyrrol-2-carboxylate (1.69 g), was obtained as a yellowish white solid.

¹H NMR (400 MHz, DMSO-d₆) δ ppm 0.32-0.41 (2H, m), 0.62-0.74 (2H, m), 1.25(3H, t, J=7.1 Hz), 1.39-1.53(1H, m), 4.17 (2H, q, J=7.1 Hz), 4.81 (2H, br. s.). 6.40 (1H, d, J=3.2 Hz), 10.31 (1H, br. s.).

### Reference Example 5

### Ethyl 2-amino-5-oxo-4,5-dihydro-1H-pyrrol-3-carboxylate

Ethyl 3-amino-3-iminopropanoate hydrochloride (3 g) obtained by a method described in a published document, Chemical and Pharmaceutical Bulletin (Chem. Pharm. Bull.), Vol. 43, p. 788 (1995), or a method pursuant to thereto, was suspended in acetonitrile (90 ml), and triethylamine (6.27 ml) and ethyl bromoacetate (3.31 g) were added sequentially to this suspension. The resulting mixture was stirred for one hour at room temperature. Ethyl acetate (180 ml) was added to the reaction mixture liquid and precipitates were filtered. Then, the filtrate was concentrated under reduced pressure to obtain an orange-colored oily substance. This oily substance was dissolved in ethanol (90 ml), and a 20% sodium ethoxide-ethanol solution (15.3 g) was added thereto. Then, the resulting mixture was stirred for 30 minutes at room temperature. Acetic acid (3.09 ml) was added thereto, and then the reaction mixture liquid was concentrated under reduced pressure to obtain an orange-colored crude product. A saturated aqueous solution of sodium hydrogen carbonate was added to the crude product, and the mixture was salted out, and then was extracted with a mixed solvent of 30% tetrahydrofuran/ethyl acetate. The organic layer was washed with saturated brine, dried over anhydrous magnesium sulfate, and then concentrated under reduced pressure to obtain an orange-colored crude solid. This crude solid was purified by chromatography, and thus the title compound (1.22 g) was obtained as a yellowish white solid.

¹H NMR (300 MHz, DMSO-d₆) δ ppm 1.16 (3H, t, J=7.2 Hz), 3.03 (2H, s), 4.01 (2H, q, J=7.2 Hz), 6.75 (2H, br. s.), 10.01 (1H, br. s.).

### Reference Example 6

### 4-(2,2,2-Trifluoroethoxy)aniline

### Reference Example 6a)

1-Fluoro-4-nitrobenzene (10.6 g) and 2,2,2-trifluoroethanol (12.0 g) were dissolved in N,N-dimethylformamide (80 ml), and potassium carbonate (15.5 g) was added to the solution. The mixture was stirred for 2 hours at 80° C. The reaction mixture was cooled to room temperature, and then ethyl acetate (100 ml) was added thereto. White precipitates were filtered, and the filtrate was concentrated under reduced pressure to obtain an orange-colored residue. The residue was dissolved again in ethyl acetate (400 ml), and the solution was washed with water and saturated brine, dried over anhydrous magnesium sulfate, and then concentrated under reduced pressure to give an orange-colored crude solid, which was washed with a mixed solvent of 10% diethyl ether/hexane to give 1-nitro-4-(2,2,2-trifluoroethoxy)benzene (15.8 g) as beige needles.

¹H NMR (400 MHz, DMSO-d₆) δ ppm 4.99 (2H, q, J=8.8 Hz), 7.30 (2H, d, J=9.3 Hz), 8.26 (2H, d, J=9.3 Hz).

### Reference Example 6b)

1-Nitro-4-(2,2,2-trifluoroethoxy)benzene (5.5 g) obtained in Reference Example (6a) was dissolved in methanol (100 ml), and 10% palladium/activated carbon (50% hydrated, 2.5 g) was added to the solution. The resulting mixture was stirred for 24 hours under a hydrogen atmosphere. Then, the palladium/activated carbon was filtered, and the filtrate was concentrated under reduced pressure to give a dark orange-colored oily residue. The residue was dissolved in ethyl acetate (200 ml), and the solution was washed with saturated brine, dried over anhydrous magnesium sulfate, and concentrated under reduced pressure to give a dark orange-colored oily residue. The residue was purified by chromatography to give the title compound, 4-(2,2,2-trifluoroethoxy)aniline (4.5 g) as an orange-colored solid.

¹H NMR (400 MHz, DMSO-d₆) δ ppm 4.53 (2H, q, J=9.0 Hz), 4.76(2H, s), 6.52 (2H, d, J=8.9 Hz), 6.75 (2H, d, J=8.9 Hz).

### Reference Example 7

### 4-(2,2-Dimethylpropoxy)aniline

A solution of 2,2-dimethylpropane (5.0 g) in N,N-dimethylformamide (20 ml) was added dropwise to a mixture of sodium hydride (60% in oil, 2.7 g) and N,N-dimethylformamide (30 ml) under ice cooling. The resulting mixture was stirred for 30 minutes at room temperature, and then a solution of 1-fluoro-4-nitrobenzene (8.8 g) in N,N-dimethylformamide (20 ml) was added dropwise to the reaction mixture liquid. The resulting mixture was stirred for 5 hours at room temperature. Subsequently, a saturated aqueous solution of ammonium chloride (100 ml) was added to the reaction mixture liquid, and the mixture was extracted with ethyl acetate. Then, the organic layer was washed with saturated brine, dried over anhydrous sodium sulfate, and then concentrated under reduced pressure. A crude product obtained therefrom was purified by chromatography, and thus 1-(2,2-dimethylpropoxy)-4-nitrobenzene (9.3 g) was obtained as a pale yellow oily substance. To this oily substance, 10% palladium/activated carbon (50% hydrated, 490 mg) and methanol (100 ml) were added, and the resulting mixture was stirred for 5 hours under a hydrogen atmosphere (40 psi). Subsequently, the reaction mixture was filtered, and the filtrate was concentrated under reduced pressure to obtain the title compound (7.2 g).

¹H NMR (400 MHz, CHLOROFORM-d) δ ppm 0.94 (9H, s), 3.32 (2H, br. s.), 3.44 (2H, s), 6.55 (2H, d, J=8.8 Hz), 6.67 (2H, d, J=8.8 Hz).

### Reference Example 8

### 4-(Cyclobutylmethoxy)aniline

A solution of cyclobutylmethanol (5.0 g) in N,N-dimethylformamide (20 ml) was added dropwise to a mixture of sodium hydride (60% in oil, 2.8 g) and N,N-dimethylformamide (30 ml) under ice cooling. The resulting mixture was stirred for 30 minutes at room temperature, and then a solution of 1-fluoro-4-nitrobenzene (9.0 g) in N,N-dimethylformamide (20 ml) was added dropwise to the reaction mixture liquid. The resulting mixture was stirred for 5 hours at room temperature. Subsequently, a saturated aqueous solution of ammonium chloride (100 ml) was added to the reaction mixture liquid, and the mixture was extracted with ethyl acetate. Then, the organic layer was washed with saturated brine, dried over anhydrous sodium sulfate, and then concentrated under reduced pressure. A crude product obtained therefrom was purified by chromatography, and thus 1-(cyclobutylmethoxy)-4-nitrobenzene (8.7 g) was obtained as a pale yellow oily substance. To this oily substance, 10% palladium/activated carbon (50% hydrated, 490 mg) and methanol (100 ml) were added, and the resulting mixture was stirred for 5 hours under a hydrogen atmosphere (40 psi). Subsequently, the reaction mixture was filtered, and the filtrate was concentrated under reduced pressure to obtain the title compound (6.4 g).

¹H NMR (400 MHz, CHLOROFORM-d) δ ppm 1.67-1.94 (4H, m), 2.00-2.10 (2H, m), 2.58-2.71.(1H, m), 3.33 (2H, br. s.), 3.77 (2H, d, J=6.8 Hz), 6.55 (2H, d, J=8.8 Hz), 6.67 (2H, d, J=8.8 Hz).

### Reference Example 9

### 4-(3,3-Dimethylbutoxy)aniline

A solution of 3,3-dimethylbutanol (5.0 g) in N,N-dimethylformamide (20 ml) was added dropwise to a mixture of sodium hydride (60% in oil, 2.4 g) and N,N-dimethylformamide (30 ml) under ice cooling. The resulting mixture was stirred for 30 minutes at room temperature, and then a solution of 1-fluoro-4-nitrobenzene (7.6 g) in N,N-dimethylformamide (20 ml) was added dropwise to the reaction mixture liquid. The resulting mixture was stirred for 5 hours at room temperature. Subsequently, a saturated aqueous solution of ammonium chloride (100 ml) was added to the reaction mixture liquid, and the mixture was extracted with ethyl acetate. Then, the organic layer was washed with saturated brine, dried over anhydrous sodium sulfate, and then concentrated under reduced pressure. A crude product obtained therefrom was purified by chromatography, and thus 1-(3,3-dimethylbutoxy)-4-nitrobenzene (9.0 g) was obtained as a pale yellow oily substance. To this oily substance, 10% palladium/activated carbon (50% hydrated, 490 mg) and methanol (100 ml) were added, and the resulting mixture was stirred for 5 hours under a hydrogen atmosphere (40 psi). Subsequently, the reaction mixture was filtered, and the filtrate was concentrated under reduced pressure to obtain the title compound (6.8 g).

¹H NMR (400 MHz, CHLOROFORM-d) δ ppm 0.90 (9H, s), 1.61 (2H, t, J=7.2 Hz), 3.87 (2H, t, J=7.2 Hz), 6.65 (2H, d, J=8.8 Hz), 6.66 (2H, d, J=8.8 Hz).

### Reference Example 10

### 1-[(2,2-Difluorocyclopropyl)methoxy]-4-nitrobenzene

A mixture of 4-nitrophenol (22 g), allyl bromide (20 g), potassium carbonate (34 g) and N,N-dimethylformamide (250 ml) was stirred for 2 hours at room temperature, and then was poured into water (100 ml), and the resultant was extracted with ethyl acetate. The organic layer was washed with saturated brine, dried over anhydrous sodium sulfate, and then concentrated under reduced pressure, to obtain 1-nitro-4-(prop-2-en-1-yloxy)benzene (30 g). 5 g of this was taken, and sodium fluoride (10 mg) was added thereto. While the mixture was stirred at 100° C., trimethylsilyl 2,2-difluoro-2-(fluorosulfonyl)acetate (3.02 g) was slowly added to the mixture dropwise at a rate of 1.6 ml/h using a syringe pump. The reaction mixture was returned to room temperature, and then was added dropwise to water (50 ml). The resulting mixture was extracted with ethyl acetate, and the organic layer was concentrated under reduced pressure to obtain a crude product. This crude product was purified by chromatography, and thus 1-[(2,2-difluorocyclopropyl)methoxy]-4-nitrobenzene (3.0 g) was obtained.

¹H NMR (400 MHz, CHLOROFORM-d) δ ppm 1.21-1.70 (1H, m), 1.53-1.70 (1H, m), 2.02-2.17 (1H, m), 4.05-4.15 (2H, m), 6.94 (2H, d, J=9.2 Hz), 8.19 (2H, d, J=9.2 Hz).

### Reference Example 11

### 4-[3,3,3-Trifluoropropoxy)aniline

A solution of 3,3,3-trifluoropropan-1-ol (5.0 g) in N,N-dimethylformamide (20 ml) was added dropwise to a mixture of sodium hydride (60% in oil, 2.1 g) and N,N-dimethylformamide (30 ml) under ice cooling. The resulting mixture was stirred for 30 minutes at room temperature, and then a solution of 1-fluoro-4-nitrobenzene (6.8 g) in N,N-dimethylformamide (20 ml) was added dropwise to the reaction mixture liquid. The resulting mixture was stirred for 5 hours at room temperature. Subsequently, a saturated aqueous solution of ammonium chloride (100 ml) was added to the reaction mixture liquid, and the mixture was extracted with ethyl acetate. Then, the organic layer was washed with saturated brine, dried over anhydrous sodium sulfate, and then concentrated under reduced pressure. A crude product obtained therefrom was purified by chromatography, and thus 1-(3,3,3-trifluoropropoxy)-4-nitrobenzene (4.2 g) was obtained as a pale yellow oily substance. To this oily substance, 10% palladium/activated carbon (50% hydrated, 490 mg) and methanol (100 ml) were added, and the resulting mixture was stirred for 5 hours under a hydrogen atmosphere (40 psi). Subsequently, the reaction mixture was filtered, and the filtrate was concentrated under reduced pressure to obtain the title compound (3.2 g).

¹H NMR (400 MHz, CHLOROFORM-d) δ ppm 2.44-2.63 (2H, m), 3.34 (2H, br. s.), 4.10 (2H, t, J=6.8 Hz), 6.62 (2H, d, J=8.8 Hz), 6.73 (2H, d, J=8.8Hz).

### Reference Example 12

### 4-Butoxyaniline

A solution of butan-1-ol (5.0 g) in N,N-dimethylformamide (20 ml) was added dropwise to a mixture of sodium hydride (60% in oil, 3.2 g) and N,N-dimethylformamide (30 ml) under ice cooling. The resulting mixture was stirred for 30 minutes at room temperature, and then a solution of 1-fluoro-4-nitrobenzene (10.5 g) in N,N-dimethylformamide (20 ml) was added dropwise to the reaction mixture liquid. The resulting mixture was stirred for 5 hours at room temperature. Subsequently, a saturated aqueous solution of ammonium chloride (100 ml) was added to the reaction mixture liquid, and the mixture was extracted with ethyl acetate. Then, the organic layer was washed with saturated brine, dried over anhydrous sodium sulfate, and then concentrated under reduced pressure. A crude product obtained therefrom was purified by chromatography, and thus 1-butoxy-4-nitrobenzene (9.8 g) was obtained as a pale yellow oily substance. To this oily substance, 10% palladium/activated carbon (50% hydrated, 490 mg) and methanol (100 ml) were added, and the resulting mixture was stirred for 5 hours under a hydrogen atmosphere (40 psi). Subsequently, the reaction mixture was filtered, and the filtrate was concentrated under reduced pressure to obtain the title compound (7.2 g).

¹H NMR (400 MHz, CHLOROFORM-d) δ ppm 0.98 (3H, t, J=7.2 Hz), 1.38-1.58 (2H, m), 1.70-1.80 (2H, m), 3.42 (2H, br. s.), 3.90 (2H, t, J=6.8 Hz), 6.65 (2H, d, J=8.8 Hz), 6.75 (2H, d, J=8.8 Hz).

### Reference Example 13

### 4-(Cyclopropylmethoxy)aniline

A solution of cyclopropylmethanol (5.0 g) in N,N-dimethylformamide (20 ml) was added dropwise to a mixture of sodium hydride (60% in oil, 3.3 g) and N,N-dimethylformamide (30 ml) under ice cooling. The resulting mixture was stirred for 30 minutes at room temperature, and then a solution of 1-fluoro-4-nitrobenzene (10.8 g) in N,N-dimethylformamide (20 ml) was added dropwise to the reaction mixture liquid. The resulting mixture was stirred for 5 hours at room temperature. Subsequently, a saturated aqueous solution of ammonium chloride (100 ml) was added to the reaction mixture liquid, and the mixture was extracted with ethyl acetate. Then, the organic layer was washed with saturated brine, dried over anhydrous sodium sulfate, and then concentrated under reduced pressure. A crude product obtained therefrom was purified by chromatography, and thus 1-(cyclopropylmethoxy)-4-nitrobenzene (10.2 g) was obtained as a pale yellow oily substance. To this compound (9.8 g), 10% palladium/activated carbon (50% hydrated, 490 mg) and methanol (100 ml) were added, and the resulting mixture was stirred for 5 hours under a hydrogen atmosphere (40 psi). Subsequently, the reaction mixture was filtered, and the filtrate was concentrated under reduced pressure to obtain the title compound (7.2 g).

¹H NMR (400 MHz, CHLOROFORM-d) δ ppm 0.18-0.30 (2H, m), 0.47-0.59 (2H, m), 1.09-1.12 (1H, m), 3.31 (2H, br. s.), 3.64 (2H, d, J=6.8 Hz), 6.56 (2H, d, J=8.8 Hz), 6.68 (2H, d, J=8.8 Hz).

### Reference Example 14

### 4-[(1-Methylcyclopropyl)methoxy]aniline

Methyl 1-methylcyclopropanecarboxylate (10 g) was dissolved in tetrahydrofuran (100 ml), and lithium aluminum hydride (5 g) was slowly added thereto under ice cooling. The resulting mixture was stirred for 4 hours at 0° C. Subsequently, water (5 ml), a 15% aqueous solution of sodium hydroxide (5 ml) and water (15 ml) were sequentially added, thereto and precipitates generated therefrom were filtered. The filtrate was concentrated under reduced pressure, and thus a crude product (5.6 g) was obtained as a pale yellow oily substance. A solution of this oily substance (5.0 g) in N,N-dimethylformamide (20 ml) was added dropwise to a mixture of sodium hydride (60% in oil, 2.8 g) and N,N-dimethylformamide (30 ml) under ice cooling. The resulting mixture was stirred for 30 minutes at room temperature, and then a solution of 1-fluoro-4-nitrobenzene (9.0 g) in N,N-dimethylformamide (20 ml) was added dropwise to the reaction mixture liquid. The resulting mixture was stirred for 5 hours at room temperature. Subsequently, a saturated aqueous solution of ammonium chloride (100 ml) was added to the reaction mixture liquid, and the mixture was extracted with ethyl acetate. Then, the organic layer was washed with saturated brine, dried over anhydrous sodium sulfate, and then concentrated under reduced pressure. A crude product obtained therefrom was purified by chromatography, and thus 1-[(1-methylcyclopropyl)methoxy]-4-nitrobenzene (8.9 g) was obtained as a pale yellow oily substance. To this oily substance, 10% palladium/activated carbon (50% hydrated, 490 mg) and methanol (80 ml) were added, and the resulting mixture was stirred for 5 hours under a hydrogen atmosphere (40 psi). Subsequently, the reaction mixture was filtered, and the filtrate was concentrated under reduced pressure to obtain the title compound (6.6 g).

¹H NMR (400 MHz, CHLOROFORM-d) δ ppm 0.28-0.37 (2H, m), 0.41-0.47 (2H, m), 1.15 (3H, s), 3.58 (2H, s), 6.55 (2H, dd, J=6.8, 2.4 Hz), 6.67 (2H, dd, J=6.8, 2.4 Hz).

### Reference Example 15

### 4-[(2-Methylcyclopropyl)methoxy]aniline

A solution of (2-methylcyclopropyl)methanol (5.0 g) in N,N-dimethylformamide (20 ml) was added dropwise to a mixture of sodium hydride (60% in oil, 2.8 g) and N,N-dimethylformamide (30 ml) under ice cooling. The resulting mixture was stirred for 30 minutes at room temperature, and then a solution of 1-fluoro-4-nitrobenzene (9.0 g) in N,N-dimethylformamide (20 ml) was added dropwise to the reaction mixture liquid. The resulting mixture was stirred for 5 hours at room temperature. Subsequently, a saturated aqueous solution of ammonium chloride (100 ml) was added to the reaction mixture liquid, and the mixture was extracted with ethyl acetate. Then, the organic layer was washed with saturated brine, dried over anhydrous sodium sulfate, and then concentrated under reduced pressure. A crude product obtained therefrom was purified by chromatography, and thus 1-[(2-methylcyclopropyl)methoxy]-4-nitrobenzene (8.5 g) was obtained as a pale yellow oily substance. To this oily substance, 10% palladium/activated carbon (50% hydrated, 490 mg) and methanol (80 ml) were added, and the resulting mixture was stirred for 5 hours under a hydrogen atmosphere (40 psi). Subsequently, the reaction mixture was filtered, and the filtrate was concentrated under reduced pressure to obtain the title compound (6.4 g).

¹N NMR (400 MHz, CHLOROFORM-d) δ ppm 0.27-0.32 (1H, m), 0.38-0.45 (1H, m), 0.62-0.71 (1H, m), 0.85-0.94 (1H, m), 1.04 (3H, d, J=6.4 Hz), 3.11 (2H, br. s.), 3.61-3.73 (2H, m), 6.58 (2H, d, J=8.8 Hz), 6.70 (2H, d, J=8.8 Hz).

### Reference Example 16

### 4-(4,4,4-Trifluorobutoxy)aniline

A solution of 4,4,4-trifluorobutan-1-ol (5.0 g) in N,N-dimethylformamide (20 ml) was added dropwise to a mixture of sodium hydride (60% in oil, 1.9 g) and N,N-dimethylformamide (30 ml) under ice cooling. The resulting mixture was stirred for 30 minutes at room temperature, and then a solution of 1-fluoro-4-nitrobenzene (6.1 g) in N,N-dimethylformamide (20 ml) was added dropwise to the reaction mixture liquid. The resulting mixture was stirred for 5 hours at room temperature. Subsequently, a saturated aqueous solution of ammonium chloride (100 ml) was added to the reaction mixture liquid, and the mixture was extracted with ethyl acetate. Then, the organic layer was washed with saturated brine, dried over anhydrous sodium sulfate, and then concentrated under reduced pressure. A crude product obtained therefrom was purified by chromatography, and thus 1-nitro-4-(4,4,4-trifluorobutoxy)benzene (6.7 g) was obtained as a pale yellow oily substance. To this oily substance, 10% palladium/activated carbon (50% hydrated, 490 mg) and methanol (80 ml) were added, and the resulting mixture was stirred for 5 hours under a hydrogen atmosphere (40 psi). Subsequently, the reaction mixture was filtered, and the filtrate was concentrated under reduced pressure to obtain the title compound (4.8 g).

¹H NMR (400 MHz, CHLOROFORM-d) δ ppm 1.94-2.04 (2H, m), 2.21-2.38 (2H, m), 3.38 (2H, br. s.), 3.94 (2H, t, J=6.0 Hz), 6.64 (2H, d, J=8.8 Hz), 6.73 (2H, d, J=8.8 Hz).

### Reference Example 17

### 4-(3-Methylbutoxy)aniline

A solution of 3-methylbutan-1-ol (5.0 g) in N,N-dimethylformamide (20 ml) was added dropwise to a mixture of sodium hydride (60% in oil, 2.7 g) and N,N-dimethylformamide (30 ml) under ice cooling. The resulting mixture was stirred for 30 minutes at room temperature, and then a solution of 1-fluoro-4-nitrobenzene (8.8 g) in N,N-dimethylformamide (20 ml) was added dropwise to the reaction mixture liquid. The resulting mixture was stirred for 5 hours at room temperature. Subsequently, a saturated aqueous solution of ammonium chloride (100 ml) was added to the reaction mixture liquid, and the mixture was extracted with ethyl acetate. Then, the organic layer was washed with saturated brine, dried over anhydrous sodium sulfate, and then concentrated under reduced pressure. A crude product obtained therefrom was purified by chromatography, and thus 1-(3-methylbutoxy)-4-nitrobenzene (8.4 g) was obtained as a pale yellow oily substance. To this oily substance, 10% palladium/activated carbon (50% hydrated, 490 mg) and methanol (80 ml) were added, and the resulting mixture was stirred for 5 hours under a hydrogen atmosphere (40 psi). Subsequently, the reaction mixture was filtered, and the filtrate was concentrated under reduced pressure to obtain the title compound (6.2 g).

¹H NMR (400 MHz, CHLOROFORM-d) δ ppm 0.81-0.95 (6H, m), 1.52-1.63 (2H, m), 1.69-1.79 (1H, m), 3.84 (2H, t, J=6.4 Hz), 6.56 (2H, d, J=8.8 Hz), 6.67 (2H, d, J=8.8 Hz).

### Reference Example 18

### 4-(Cyclopropylmethoxy)-2-fluoroaniline

### Reference Example 18a)

A mixture of 3-fluoro-4-nitrophenol (8.20 g), (bromomethyl)cyclopropane (8.46 g), potassium carbonate (8.65 g) and N,N-dimethylformamide (100 ml) was stirred for 15 hours at 80° C. The reaction solution was returned to room temperature, and then the solvent was distilled off under reduced pressure. The residue was diluted with ethyl acetate, and the dilution was washed with water and saturated brine, dried over anhydrous magnesium sulfate, and then concentrated under reduced pressure. The resulting residue was purified by chromatography, and thus 4-(cyclopropylmethoxy)-2-fluoro-1-nitrobenzene (9.6 g) was obtained as a yellow oily substance.

¹H NMR (300 MHz, CHLOROFORM-d) δ ppm 0.35-0.42 (2H, m), 0.66-0.74 (2H, m), 1.22-1.36 (1H, m), 3.88 (2H, d, J=7.2 Hz), 6.67-6.79 (2H, m), 8.03-8.14 (1H, m).

### Reference Example 18b)

A mixture of 4-(cyclopropylmethoxy)-2-fluoro-1-nitrobenzene (9.50 g) obtained in Reference Example (18a), 10% palladium/activated carbon (50% hydrated, 4.75 g) and methanol (200 ml) was stirred for 15 hours at normal pressure and room temperature under a hydrogen atmosphere. Subsequently, the reaction solution was filtered, and the resulting filtrate was concentrated under reduced pressure, and thus the title compound, 4-(cyclopropylmethoxy)-2-fluoroaniline (8.0 g), was obtained as a pale yellow oily substance.

¹H NMR (300 MHz, CHLOROFORM-d) δ ppm 0.27-0.36 (2H, m), 0.58-0.67 (2H, m), 1.15-1.31 (1H, m), 3.42 (2H, br. s.), 3.70 (2H, d, J=6.8 Hz), 6.51-6.74 (2H, m), 7.12-7.29 (1H, m).

### Reference Example 19

### 4-(2-Cyclopropylethoxy)aniline

### Reference Example 19a)

A mixture of sodium hydride (60% in oil, 3.2 g), 2-cyclopropylethanol (7.67 g) and N,N-dimethylformamide (150 ml) in an ice water bath was stirred for 30 minutes, and then a solution of 1-fluoro-4-nitrobenzene (10.0 g) in N,N-dimethylformamide (30 ml) was added thereto. The resulting mixture was stirred for 4 hours at 80° C., cooled to room temperature, and then concentrated under reduced pressure. Water was added to the residue, and then the mixture was extracted with ethyl acetate. The organic layer was washed with water and saturated brine, dried over anhydrous sodium sulfate, and then concentrated under reduced pressure. The residue was purified by chromatography, and thus 1-(2-cyclopropylethoxy)-4-nitrobenzene (14.3 g) was obtained as a brown oily substance.

¹H NMR (300 MHz, CHLOROFORM-d) δ ppm 0.11-0.17 (2H, m), 0.48-0.55 (2H, m), 0.78-0.93 (1H, m), 1.68-1.76 (2H, m), 4.13 (2H, t, J=6.6 Hz), 6.96 (2H, d, J=9.3 Hz), 8.20 (2H, d, J=9.3 Hz).

### Reference Example 19b)

A mixture of 1-(2-cyclopropylethoxy)-4-nitrobenzene (14.3 g) obtained in Reference Example (19a), 10% palladium/activated carbon (50% hydrated, 1.5 g) and methanol (300 ml) was stirred overnight at normal pressure and room temperature under a hydrogen atmosphere. The reaction mixture was filtered, and then the filtrate was concentrated under reduced pressure. The residue was purified by chromatography, and thus the title compound, 4-(2-cyclopropylethoxy)aniline (11.4 g), was obtained as a brown oily substance.

¹1H NMR (300 MHz, CHLOROFORM-d) δ ppm 0.07-0.13 (2H, m), 0.43-0.51 (2H, m), 0.75-0.90 (1H, m), 1.64 (2H, q, J=6.8 Hz), 3.40 (2H, br. s.), 3.96 (2H, t, J=6.8 Hz), 6.64 (2H, d, J=6.7 Hz), 6.75 (2H, d, J=6.7 Hz).

### Reference Example 20

### 4-[(2,2,2-Trifluoroethoxy)methyl]aniline

### Reference Example 20a)

To a mixture of sodium hydride (60% in oil, 2.8 g) and N,N-dimethylformamide (10 ml) in an ice water bath, a mixture of 2,2,2-trifluoroethanol (7.28 ml) and N,N-dimethylformamide (20 ml) was added dropwise. The resulting mixture was stirred for 30 minutes, and then a solution of 1-(bromomethyl)-4-nitrobenzene (15.1 g) in N,N-dimethylformamide (10 ml) was added thereto. The resulting mixture was stirred overnight at room temperature, and then was concentrated under reduced pressure. A 5% aqueous solution of citric acid and ethyl acetate were added to the residue, and then insoluble substances were filtered. The filtrate was extracted with ethyl acetate. The organic layer was washed with water and saturated brine, dried over anhydrous sodium sulfate, and then concentrated under reduced pressure. The residue was purified by chromatography, and thus 1-nitro-4-[(2,2,2-trifluoroethoxy)methyl]benzene (6.28 g) was obtained as a brown oily substance.

¹H NMR (300 MHz, CHLOROFORM-d) δ ppm 3.92 (2H, q, J=8.5 Hz), 4.79 (2H, s), 7.52 (2H, d, J=8.6 Hz), 8.24 (2H, d, J=8.6 Hz).

### Reference Example 20b)

A mixture of 1-nitro-4-[(2,2,2-trifluoroethoxy)methyl]benzene (6.28 g) obtained in Reference Example (20a), 10% palladium/activated carbon (50% hydrated, 1 g) and methanol (150 ml) was stirred overnight at normal pressure and room temperature under a hydrogen atmosphere. The reaction mixture was filtered, and then the filtrate was concentrated under reduced pressure. The residue was purified by chromatography, and thus the title compound, 4-[(2,2,2-trifluoroethoxy)methyl]aniline (4.66 g), was obtained as a brown oily substance.

¹H NMR (300 MHz, CHLOROFORM-d) δ ppm 3.71 (2H, br. s.), 3.76 (2H, q, J=8.8 Hz), 4.55 (2H, s), 6.67 (2H, d, J=8.5 Hz), 7.13 (2H, d, J=8.5 Hz).

### Reference Example 21

### 4-(Cyclopropylmethoxy)-3-methylaniline

### Reference Example 21a)

A mixture of 2-methyl-4-nitrophenol (5.00 g), (bromomethyl)cyclopropane (3.80 ml), potassium carbonate (5.40 g) and N,N-dimethylformamide (50 ml) was stirred for 3 days at room temperature, and then was concentrated under reduced pressure. Water was added to the residue, and then the mixture was extracted with ethyl acetate. The organic layer was washed with a 5% aqueous solution of sodium carbonate, water and saturated brine, dried over anhydrous sodium sulfate, and then concentrated under reduced pressure. The residue was purified by chromatography, and thus 1-(cyclopropylmethoxy)-2-methyl-4-nitrobenzene (6.63 g) was obtained as a yellow oily substance. ¹H NMR (300 MHz, DMSO-d₆) δ ppm 0.33-0.42 (2H, m), 0.56-0.64 (2H, m), 1.20-1.35 (1H, m), 2.25 (3H, s), 4.00 (2H, d, J=7.0 Hz), 7.08-7.16 (1H, m), 8.05-8.13 (2H, m).

### Reference Example 21b)

A mixture of 1-(cyclopropylmethoxy)-2-methyl-4-nitrobenzene (6.63 g) obtained in Reference Example (21a), 10% palladium/activated carbon (50% hydrated, 0.40 g), ethanol (150 ml) and methanol (50 ml) was stirred overnight at normal pressure and room temperature under a hydrogen atmosphere. The reaction mixture was filtered, and then the filtrate was concentrated under reduced pressure. The residue was purified by chromatography, and thus the title compound, 4-(cyclopropylmethoxy)-3-methylaniline (5.62 g), was obtained as a yellow oily substance.

¹H NMR (300 MHz, DMSO-d₆) δ ppm 0.23-0.31 (2H, m), 0.47-0.55 (2H, m), 1.08-1.22 (1H, m), 2.05 (3H, s), 3.64 (2H, d, J=6.6 Hz), 4.50 (2H, s), 6.31 (1H, dd, J=8.5, 2.6 Hz), 6.38 (1H, d, J=2.6 Hz), 6.59 (1H, d, J=8.5 Hz).

### Reference Example 22

### 3-Chloro-4-(cyclopropylmethoxy)aniline

### Reference Example 22a)

A mixture of 2-chloro-4-nitrophenol (5.66 g), (bromomethyl)cyclopropane (3.80 ml), potassium carbonate (5.40 g) and N,N-dimethylformamide (50 ml) was stirred for 3 days at room temperature, and then was concentrated under reduced pressure. Water was added to the residue, and then the mixture was extracted with ethyl acetate. The organic layer was washed with a 5% aqueous solution of sodium carbonate, water and saturated brine, dried over anhydrous sodium sulfate, and then concentrated under reduced pressure. The residue was purified by chromatography, and thus 2-chloro-1-(cyclopropylmethoxy)-4-nitrobenzene (6.22 g) was obtained as a yellow oily substance.

¹H NMR (300 MHz, DMSO-d₆) δ ppm 0.36-0.44 (2H, m), 0.58-0.66 (2H, m), 1.22-1.37 (1H, m), 4.10 (2H, d, J=7.0 Hz), 7.34 (1H, d, J=9.2 Hz), 8.21 (1H, dd, J=9.2, 2.7 Hz), 8.31 (1H, d, J=2.7 Hz).

### Reference Example 22b)

To a mixture of 2-chloro-1-(cyclopropylmethoxy)-4-nitrobenzene (6.22 g) obtained in Reference Example (22a) and a 90% ethanol solution (200 ml) of calcium chloride (1.0 g) at 90° C., reduced iron (10.0 g) was added in several divided portions. The resulting mixture was heated to reflux overnight, and then was cooled to room temperature. Insoluble substances were filtered through Celite, and the filtrate was concentrated under reduced pressure. Water was added to the residue, and then the mixture was extracted with ethyl acetate. The organic layer was washed with water and saturated brine, dried over anhydrous sodium sulfate, and then concentrated under reduced pressure. The residue was purified by chromatography, and thus the title compound, 3-chloro-4-(cyclopropylmethoxy)aniline (5.59 g), was obtained as a pale brown solid.

¹H NMR (300 MHz, DMSO-d₆) δ ppm 0.23-0.32 (2H, m), 0.48-0.57 (2H, m), 1.08-1.24 (1H, m), 3.71 (2H, d, J=6.8 Hz), 4.89 (2H, s), 6.45 (1H, dd, J=8.7, 2.6 Hz), 6.62 (1H, d, J=2.6 Hz), 6.82 (1H, d, J=8.7 Hz).

### Reference Example 23

### 3-Chloro-4-(2,2,2-trifluoroethoxy)aniline

### Reference Example 23a)

A mixture of 2-chloro-1-fluoro-4-nitrobenzene (5.00 g), 2,2,2-trifluoroethanol (4.56 g), potassium carbonate (5.91 g) and N,N-dimethylformamide (30 ml) was stirred for 2 hours at 80° C. The reaction solution was returned to room temperature, and then the solvent was distilled off under reduced pressure. The residue was diluted with ethyl acetate, and the dilution was washed with water and saturated brine, dried over anhydrous magnesium sulfate, and then concentrated under reduced pressure. The resulting residue was purified by chromatography, and thus 2-chloro-4-nitro-1-(2,2,2-trifluoroethoxy)benzene (6.77 g) was obtained as a white powder.

¹H NMR (300 MHz, CHLOROFORM-d) δ ppm 4.53 (2H, q, J=7.7 Hz), 7.04 (1H, d, J=9.1 Hz), 8.19 (1H, dd, J=9.1, 2.7 Hz), 8.34 (1H, d, J=2.7 Hz).

### Reference Example 23b)

A mixture of 2-chloro-4-nitro-1-(2,2,2-trifluoroethoxy)benzene (6.50 g) obtained in Reference Example (23a), reduced iron (7.09 g), calcium chloride (1.41 g), ethanol (100 ml) and water (10 ml) was heated to reflux for 6 hours. The reaction solution was returned to room temperature and filtered, and then the filtrate was concentrated under reduced pressure. The residue was diluted with ethyl acetate, and the dilution was washed with water and saturated brine, dried over anhydrous magnesium sulfate, and then concentrated under reduced pressure. The resulting residue was purified by chromatography, and thus the title compound, 3-chloro-4-(2,2,2-trifluoroethoxy)aniline (4.00 g), was obtained as a pale yellow oily substance.

¹H NMR (300 MHz, CHLOROFORM-d) δ ppm 3.60 (2H, br. s.), 4.29 (2H, q, J=8.3 Hz), 6.52 (1H, dd, J=8.6, 2.9 Hz), 6.72 (1H, d, J=2.9 Hz), 6.87 (1H, d, J=8.6 Hz).

### Reference Example 24

### 3-Fluoro-4-(2,2,2-trifluoroethoxy)aniline

### Reference Example 24a)

A mixture of 1,2-difluoro-4-nitrobenzene (5.00 g), 2,2,2-trifluoroethanol (5.03 g), potassium carbonate (6.51 g) and N,N-dimethylformamide (30 ml) was stirred for 2 hours at 80° C. The reaction solution was returned to room temperature, and then the solvent was distilled off under reduced pressure. The residue was diluted with ethyl acetate, and the dilution was washed with water and saturated brine, dried over anhydrous magnesium sulfate, and then concentrated under reduced pressure. The resulting residue was purified by chromatography, and thus 2-fluoro-4-nitro-1-(2,2,2-trifluoroethoxy)benzene (7.29 g) was obtained as a pale yellow powder.

¹H NMR (300 MHz, CHLOROFORM-d) δ ppm 4.55 (2H, q, J=7.8 Hz), 7.08-7.18 (1H, m), 8.01-8.12 (2H, m).

### Reference Example 24b)

A mixture of 2-fluoro-4-nitro-1-(2,2,2-trifluoroethoxy)benzene (7.00 g) obtained in Reference Example (24a), reduced iron (8.18 g), calcium chloride (11.63 g), ethanol (100 ml) and water (10 ml) was heated to reflux for 6 hours. The reaction solution was returned to room temperature and filtered, and then the filtrate was concentrated under reduced pressure. The residue was diluted with ethyl acetate, and the dilution was washed with water and saturated brine, dried over anhydrous magnesium sulfate, and then concentrated under reduced pressure. The resulting residue was purified by chromatography, and thus the title compound, 3-fluoro-4-(2,2,2-trifluoroethoxy)aniline (5.51 g), was obtained as a pale yellow oily substance.

¹H NMR (300 MHz, CHLOROFORM-d) δ ppm 3.62 (2H, br. s.), 4.30 (2H, q, J=8.3 Hz), 6.35 (1H, ddd, J=8.7, 2.6, 1.3 Hz), 6.45 (1H, dd, J=12.6, 2.6 Hz), 6.88 (1H, t, J=8.7 Hz).

### Reference Example 25

### 3-Methyl-4-(2,2,2-trifluoroethoxy)aniline

### Reference Example 25a)

A mixture of 1-fluoro-2-methyl-4-nitrobenzene (5.00 g), 2,2,2-trifluoroethanol (5.16 g), potassium carbonate (6.70 g) and N,N-dimethylformamide (30 ml) was stirred for 2 hours at 80° C. The reaction solution was returned to room temperature, and then the solvent was distilled off under reduced pressure. The residue was diluted with ethyl acetate, and the dilution was washed with water and saturated brine, dried over anhydrous magnesium sulfate, and then concentrated under reduced pressure. The resulting residue was purified by chromatography, and thus 2-methyl-4-nitro-1-(2,2,2-trifluoroethoxy)benzene (7.54 g) was obtained as a pale yellow powder.

¹H NMR (300 MHz, CHLOROFORM-d) δ ppm 2.34 (3H, s), 4.47 (2H, q, J=7.8 Hz), 6.85 (1H, d, J=9.1 Hz), 8.06-8.16 (2H, m).

### Reference Example 25b)

2-Methyl-4-nitro-1-(2,2,2-trifluoroethoxy)benzene (7.54 g) obtained in Reference Example (25a), reduced iron (8.93 g), calcium chloride (1.78 g), ethanol (100 ml) and water (10 ml) were heated to reflux for 6 hours. The reaction solution was returned to room temperature and filtered, and then the filtrate was concentrated under reduced pressure. The residue was diluted with ethyl acetate, and the dilution was washed with water and saturated brine, dried over anhydrous magnesium sulfate, and then concentrated under reduced pressure. The resulting residue was purified by chromatography, and thus the title compound, 3-methyl-4-(2,2,2-trifluoroethoxy)aniline (5.84 g), was obtained as a white powder.

¹H NMR (300 MHz, CHLOROFORM-d) δ ppm 2.19 (3H, s), 3.45 (2H, br. s.), 4.24 (2H, q, J=8.3 Hz), 6.43-6.50 (1H, m), 6.53 (1H, d, J=3.0 Hz), 6.66 (1H, d, J=8.3 Hz).

### Reference Example 26

### 1-Nitro-4-[(1E)-3,3,3-trifluoroprop-1-en-1-yl]benzene

To a mixture of (4-nitrobenzyl)(triphenyl)phosphonium bromide (35.0 g), potassium tert-butoxide (8.38 g) and N,N-dimethylformamide (366 ml) in an ice water bath, trifluoroacetaldehyde produced at 80° C. from 1-ethoxy-2,2,2-trifluoroethanol (73.8 g), sulfuric acid (44.9 ml) and diphosphorus pentoxide (73.7 g), was added through a cannula. The resulting mixture was stirred for 24 hours at 100° C., and then was cooled to room temperature. Then, the mixture was poured into water, and the resultant was extracted with ethyl acetate. The organic layer was dried over anhydrous sodium sulfate, and then concentrated under reduced pressure. The residue was purified by chromatography, and thus 1-nitro-4-[(1E)-3,3,3-trifluoroprop-1-en-1-yl]benzene (9.40 g) was obtained as a yellow solid.

¹H NMR (400 MHz, CHLOROFORM-d) δ ppm 6.32-6.41 (1H, m), 7.23 (1H, d, J=16.0 Hz), 7.63 (2H, d, J=8.4 Hz), 8.27 (2H, d, J=8.4 Hz).

### Reference Example 27

### 4-(3,3,3-Trifluoropropyl)aniline hydrochloride

A mixture of 1-nitro-4-[(1E)-3,3,3-trifluoroprop-1-en-1-yl]benzene (9.40 g) obtained in Reference Example 26, 5% palladium/activated carbon (9.21 g) and methanol (430 ml) was stirred for 2 hours at normal pressure and room temperature under a hydrogen atmosphere. The reaction mixture was filtered, and then the filtrate was concentrated under reduced pressure. The residue was purified by chromatography, and thus 4-(3,3,3-trifluoropropyl)aniline (6.49 g) was obtained as an oily substance. To a diethyl ether (40 ml) solution of 4-(3,3,3-trifluoropropyl)aniline (6.49 g) in an ice water bath, a 4 M hydrogen chloride/1,4-dioxane solution (8.64 ml) was added dropwise. The resulting mixture was stirred for 10 minutes at room temperature, and then a solid precipitated therefrom was collected by filtration. Thus, the title compound, 4-(3,3,3-trifluoropropyl)aniline hydrochloride (6.85 g), was obtained as a yellow solid.

¹H NMR (400 MHz, DMSO-d₆) δ ppm 2.53-2.66 (2H, m), 2.82-2.86 (2H, m), 7.28 (2H, d, J=8.4 Hz), 7.39 (2H, d, J=8.4 Hz), 10.13 (3H, br. s.).

### Reference Example 28

### 4-(2-Cyclopropylethyl)aniline hydrochloride

### Reference Example 28a)

To a mixture of (cyclopropylmethyl)(triphenyl)phosphonium bromide (43.4 g) and tetrahydrofuran (220 ml), potassium tert-butoxide (12.3 g) was added in several divided portions at room temperature. The resulting mixture was heated to reflux for 30 minutes, and then 4-nitrobenzaldehyde (11.0 g) was added thereto in an ice water bath. The resulting mixture was heated to reflux for 2.5 hours, and then was cooled to room temperature. Subsequently, water was added to the mixture, and the resultant was extracted with ethyl acetate. The organic layer was washed with brine, dried over anhydrous magnesium sulfate, and then concentrated under reduced pressure. The residue was purified by chromatography, and thus a mixture of cis- and trans-1-(2-cyclopropylethenyl)-4-nitrobenzene (13.3 g) was obtained as a yellow oily substance.

¹H NMR (400 MHz, CHLOROFORM-d) δ ppm 0.53-0.62 (2H, m), 0.88-0.94 (2H, m), 1.59-1.67 and 1.80-1.88 (1H, m, trans and cis), 5.26 and 5.91 (1H, dd, J_{cis}=11.6, 10.4 Hz and Jₜᵣₐₙₛ=15.6, 9.2 Hz), 6.37 and 6.52 (1H, d, J_{cis}=11.6 Hz and Jₜᵣₐₙₛ=15.6), 7.39 and 7.56 (2H, d, trans and cis, J=10.8 Hz), 8.14 and 8.20 (2H, d, trans and cis, J=10.8 Hz). *cis-trans mixture (4:3 ratio).

### Reference Example 28b)

A mixture of 1-(2-cyclopropylethenyl)-4-nitrobenzene (13.2 g) obtained in Reference Example (28a), 5% palladium/activated carbon (14.9 g) and methanol (350 ml) was stirred for 4 days at room temperature under a hydrogen atmosphere (150 psi). The reaction mixture was filtered, and then the filtrate was concentrated under reduced pressure. The residue was purified by chromatography, and thus 4-(2-cyclopropylethyl)aniline (5.05 g) was obtained as an oily substance. To a diethyl ether (40 ml) solution of 4-(2-cyclopropylethyl)aniline (5.05 g) in an ice water bath, a 4 M hydrogen chloride/1,4-dioxane solution (9.40 ml) was added dropwise. The resulting mixture was stirred for 10 minutes at room temperature, and then a solid precipitated therefrom was collected by filtration. Thus, the title compound, 4-(2-cyclopropylethyl)aniline hydrochloride (5.10 g), was obtained as a white solid.

¹H NMR (400 MHz, DMSO-d₆) δ ppm 0.01-0.09 (2H, m), 0.36-0.42 (2H, m), 0.62-0.72 (1H, m), 1.45-1.52 (2H, m), 2.65-2.69 (2H, m), 7.25 (2H, d, J=8.4 Hz), 7.31 (2H, d, J=8.4 Hz), 10.09 (3H, br. s.).

### Reference Example 29

### 4-(2,2-Difluoroethoxy)aniline

### Reference Example 29a)

To a mixture of sodium hydride (60% in oil, 2.0 g) and N,N-dimethylformamide (30 ml) in an ice water bath, a mixture of 2,2-difluoroethanol (4.51 g) and N,N-dimethylformamide (50 ml) was added dropwise. The resulting mixture was stirred for 30 minutes, and then 1-fluoro-4-nitrobenzene (7.05 g) was added thereto. The mixture was stirred for 3 hours at room temperature, and then was concentrated under reduced pressure. Water was added to the residue. A solid precipitated therefrom was collected by filtration, washed with water and hexane, and then dried. Thus, 1-(2,2-difluoroethoxy)-4-nitrobenzene (9.97 g) was obtained as a yellow solid.

¹H NMR (300 MHz, CHLOROFORM-d) δ ppm 4.28 (2H, td, J=12.9, 4.2 Hz), 6.13 (1H, tt, J=54.8, 4.2 Hz), 7.01 (2H, d, J=9.3 Hz), 8.24 (2H, d, J=9.3 Hz).

### Reference Example 29b)

A mixture of 1-(2,2-difluoroethoxy)-4-nitrobenzene (9.97 g) obtained in Reference Example (29a), 10% palladium/activated carbon (50% hydrated, 2.0 g) and methanol (300 ml) was stirred overnight at normal pressure and room temperature under a hydrogen atmosphere. The reaction mixture was filtered, and then the filtrate was concentrated under reduced pressure. The residue was purified by chromatography, and thus the title compound, 4-(2,2-difluoroethoxy)aniline (8.46 g), was obtained as a brown oily substance.

¹H NMR (300 MHz, CHLOROFORM-d) δ ppm 3.48 (2H, br. s.), 4.10 (2H, td, J=13.3, 4.2 Hz), 6.04 (1H, tt, J=55.3, 4.2 Hz), 6.64 (2H, d, J=9.0 Hz), 6.76 (2H, d, J=9.0 Hz).

### Reference Example 30

### 4-(2,2,3,3,3-Pentafluoropropoxy)aniline

### Reference Example 30a)

A mixture of 1-fluoro-4-nitrobenzene (7.05 g), 2,2,3,3,3-pentafluoropropan-1-ol (11.3 g), potassium carbonate (8.29 g) and N,N-dimethylformamide (150 ml) was stirred overnight at 60° C., and then was concentrated under reduced pressure. Water was added to the residue, and then a solid precipitated therefrom was collected by filtration, washed with water and hexane, and then dried. Thus, 1-nitro-4-(2,2,3,3,3-pentafluoropropoxy)benzene (13.3 g) was obtained as a yellow solid.

¹H NMR (300 MHz, CHLOROFORM-d) δ ppm 4.52 (2H, tq, J=11.9, 1.1 Hz), 7.05 (2H, d, J=9.3 Hz), 8.26 (2H, d, J=9.3 Hz).

### Reference Example 30b)

A mixture of 1-nitro-4-(2,2,3,3,3-pentafluoropropoxy)benzene (13.3 g) obtained in Reference Example (30a), 10% palladium/activated carbon (50% hydrated, 2.0 g) and methanol (300 ml) was stirred overnight at normal pressure and room temperature under a hydrogen atmosphere. The reaction mixture was filtered, and then the filtrate was concentrated under reduced pressure. The residue was purified by chromatography, and thus the title compound, 4-(2,2,3,3,3-pentafluoropropoxy)aniline (11.6 g), was obtained as a brown oily substance.

¹H NMR (300 MHz, CHLOROFORM-d) δ ppm 3.51 (2H, br. s.), 4.33 (2H, tq, J=12.6, 1.1 Hz), 6.64 (2H, d, J=8.9 Hz), 6.79 (2H, d, J=8.9 Hz).

### Reference Example 31

### Ethyl 3-isothiocyanato-1H-pyrrol-2-carboxylate

Ethyl 3-amino-1H-pyrrol-2-carboxylate (1.88 g) obtained by a method described in a published document, Journal of Organic Chemistry (J. Org. Chem.), Vol. 64, p. 8411 (1999), or a method pursuant to thereto, was dissolved in tetrahydrofuran (60 ml), and 1,1′-carbonothioyldipyridin-2(1H)-one (3.12 g) was added to the solution under ice cooling. The resulting mixture was stirred for one hour at room temperature. Subsequently, silica gel (30 g) was added to the reaction mixture liquid, and the solvent was distilled off under reduced pressure. The resulting mixture was purified by chromatography, and thus the title compound (1.96 g) was obtained as a white solid.

¹H NMR (300 MHz, DMSO-d₆) δ ppm 1.32 (3H, t, J=6.9 Hz), 4.28 (2H, q, J=6.9 Hz), 6.33 (1H, d, J=2.7 Hz), 7.02 (1H, d, J=2.7 Hz), 12.26 (1H, br. s.).

### Reference Example 32

### Ethyl 3-isothiocyanato-5-methyl-1H-pyrrol-2-carboxylate

Ethyl 3-amino-5-methyl-1H-pyrrol-2-carboxylate (505 mg) obtained by the method of Reference Example 1, or a method pursuant to thereto, was dissolved in tetrahydrofuran (15 ml), and 1,1′-carbonothioyldipyridin-2(1H)-one (906 mg) was added thereto. The mixture was stirred for 18 hours at room temperature. Subsequently, silica gel was added to the reaction mixture liquid, and the solvent was distilled off under reduced pressure. The resulting mixture was purified by chromatography, and thus the title compound (411 mg) was obtained as a white solid.

¹H NMR (300 MHz, DMSO-d₆) δ ppm 1.31 (3H, t, J=7.2 Hz), 2.17 (3H, s), 4.26 (2H, q, J=7.2 Hz), 6.06 (1H, s), 12.00 (1H, br. s.).

### Reference Example 33

### Ethyl 3-isothiocyanato-4-methyl-1H-pyrrol-2-carboxylate

Ethyl 3-amino-4-methyl-1H-pyrrol-2-carboxylate (238 mg) obtained by the method of Reference Example 2, or a method pursuant to thereto, was dissolved in tetrahydrofuran (10 ml), and 1,1′-carbonothioyldipyridin-2(1H)-one (559 mg) was added thereto. The mixture was stirred for 18 hours at room temperature. Subsequently, silica gel was added to the reaction mixture liquid, and the solvent was distilled off under reduced pressure. The resulting mixture was purified by chromatography, and thus the title compound (272 mg) was obtained as a white solid.

¹H NMR (300 MHz, DMSO-d₆) δ ppm 1.31 (3H, t, J=7.2 Hz), 2.01 (3H, s), 4.27 (2H, q, J=7.2 Hz), 6.87 (1H, s), 12.01 (1H, br. s.).

### Reference Example 34

### Ethyl 4-ethyl-3-isothiocyanato-1H-pyrrol-2-carboxylate

Ethyl 3-amino-4-ethyl-1H-pyrrol-2-carboxylate (1.14 g) obtained by the method of Reference Example 3, or a method pursuant to thereto, was dissolved in tetrahydrofuran (30 ml), and 1,1′-carbonothioyldipyridin-2(1H)-one (1.60 g) was added thereto. The mixture was stirred for 2 hours at room temperature. Subsequently, silica gel was added to the reaction mixture liquid, and the solvent was distilled off under reduced pressure. The resulting mixture was purified by chromatography, and thus the title compound (1.27 g) was obtained as a white solid.

¹H NMR (400 MHz, DMSO-d₆) δ ppm 1.13 (3H, t, J=7.6 Hz), 1.31 (3H, t, J=7.1 Hz), 2.42 (2H, q, J=7.6 Hz), 4.27 (2H, q, J=7.1 Hz), 6.88 (1H, s), 12.06 (1H, br. s.).

### Reference Example 35

### Ethyl 4-cyclopropyl-3-isothiocyanato-1H-pyrrol-2-carboxylate

Ethyl 3-amino-4-cyclopropyl-1H-pyrrol-2-carboxylate (1.05 g) obtained by the method of Reference Example 4, or a method pursuant to thereto, was dissolved in tetrahydrofuran (30 ml), and 1,1′-carbonothioyldipyridin-2(1H)-one (1.38 g) was added thereto. The mixture was stirred for 12 hours at room temperature. Subsequently, silica gel was added to the reaction mixture liquid, and the solvent was distilled off under reduced pressure. The resulting mixture was purified by chromatography, and thus the title compound (1.18 g) was obtained as a white solid.

¹H NMR (300 MHz, DMSO-d₆) δ ppm 0.52-0.60 (2H, m), 0.79-0.88 (2H, m), 1.31 (3H, t, J=7.1 Hz), 1.58-1.70 (1H, m), 4.26 (2H, q, J=7.1 Hz), 6.77 (1H, s), 12.03 (1H, br. s.).

### Reference Example 36

### Ethyl 2-isothiocyanato-1H-pyrrol-3-carboxylate

Ethyl 2-amino-1H-pyrrol-3-carbovlate (1.54 g) obtained by a method described in a published document, Journal of Heterocyclic Chemistry (J. Heterocyclic Chem.), Vol. 23, p. 1555 (1986), or a method pursuant to thereto, was dissolved in tetrahydrofuran (100 ml), and 1,1′-carbonothioyldipyridin-2(1H)-one (2.55 g) was added to the solution. The mixture was stirred for one hour at room temperature. Then, silica gel was added to the reaction mixture and the solvent was evaporated under reduced pressure. The residue was purified by chromatography to give the title compound (1.57 g) as a white solid.

¹H NMR (300 MHz, DMSO-d₆) δ ppm 1.29 (3H, t, J=7.1 Hz), 4.23 (2H, q, J=7.1 Hz), 6.41 (1H, dd, J=3.0, 1.7 Hz), 6.73 (1H, dd, J=3.0, 1.7 Hz), 12.18 (1H, br. s.).

### Reference Example 37

### 1-Isothiocyanato-4-(2,2,2-trifluoroethoxy)benzene

4-(2,2,2-Trifluoroethoxy)aniline (2.51 g) was dissolved in tetrahydrofuran (25 ml), and 1,1′-carbonothioyldipyridin-2(1H)-one (3.35 g) was added to the solution. The mixture was stirred for one hour at room temperature. Then, silica gel was added to the reaction mixture and the solvent was evaporated under reduced pressure. The residue was purified by chromatography to give the title compound (2.85 g) was obtained as white needles.

¹H NMR (300 MHz, DMSO-d₆) δ ppm 4.82 (2H, q, J=8.8 Hz), 7.13 (2H, d, J=9.1 Hz), 7.45 (2H, d, J=9.1 Hz).

### Reference Example 38

### 4-(Cyclopropylmethoxy)-3-fluoroaniline

### Reference Example 38a)

A mixture of 2-fluoro-4-nitrophenol (5.00 g), (bromomethyl)cyclopropane (5.16 g), potassium carbonate (5.28 g) and N,N-dimethylformamide (100 ml) was stirred for 15 hours at 80° C. The reaction solution was returned to room temperature, and then the solvent was distilled off under reduced pressure. The residue was diluted with ethyl acetate, and the dilution was washed with water and saturated brine, dried over anhydrous magnesium sulfate, and then concentrated under reduced pressure. The resulting residue was purified by chromatography, and thus 1-(cyclopropylmethoxy)-2-fluoro-4-nitrobenzene (6.33 g) was obtained as a pale yellow oily substance.

¹H NMR (300 MHz, CHLOROFORM-d) δ ppm 0.37-0.45 (2H, m), 0.67-0.77 (2H, m), 1.25-1.42 (1H, m), 3.99 (2H, d, J=7.2 Hz), 7.00 (1H, t, J=8.3 Hz), 7.95-8.07 (2H, m).

### Reference Example 38b)

A mixture of 1-(cyclopropylmethoxy)-2-fluoro-4-nitrobenzene (6.30 g) obtained in Reference Example (38a), 10% palladium/activated carbon (50% hydrated, 1.2 g) and methanol (100 ml) was stirred for 15 hours at normal pressure and room temperature under a hydrogen atmosphere. Subsequently, the reaction solution was filtered, and the resulting filtrate was concentrated under reduced pressure. Thus, the title compound, 4-(cyclopropylmethoxy)-3-fluoroaniline (5.40 g), was obtained as a pale yellow oily substance.

¹H NMR (300 MHz, CHLOROFORM-d) δ ppm 0.26-0.38 (2H, m), 0.53-0.69 (2H, m), 1.18-1.35 (1H, m), 3.51 (2H, br. s.), 3.77 (2H, d, J=7.2 Hz), 6.31-6.41 (1H, m), 6.45 (1H, dd, J=12.9, 2.7 Hz), 6.80 (1H, t, J=8.9 Hz).

### Reference Example 39

### 2-Fluoro-4-isothiocyanato-1-(2,2,2-trifluoroethoxy)benzene

1,1′-Carbonothioyldipyridin-2(1H)-one (8.36 g) was added to a tetrahydrofuran (100 ml) solution of 3-fluoro-4-(2,2,2-trifluoroethoxy)aniline (6.27 g) obtained by the method of Reference Example 24, or a method pursuant to thereto at room temperature, and the resulting mixture was stirred for 2 hours. The solvent was distilled off, and the resulting residue was purified by chromatography, and thus the title compound (6.76 g) was obtained as a white powder.

¹H NMR (300 MHz, DMSO-d₆) δ ppm 4.90 (2H, q, J=8.9 Hz), 7.30-7.40 (2H, m), 7.52-7.61 (1H, m).

### Reference Example 40

### 2-Chloro-N-(2-cyanoethyl)acetamide

3-Aminopropanenitrile (3.5 g) and triethylamine (10.5 ml) were dissolved in tetrahydrofuran (60 ml), and chloroacetyl chloride (6.2 g) dissolved in tetrahydrofuran (30 ml) was added dropwise to the solution over 5 minutes under ice cooling. The mixture was stirred for one hour at room temperature. Subsequently, diethyl ether (100 ml) was added to the reaction solution, and precipitates generated therefrom were filtered. The filtrate was concentrated under reduced pressure to obtain a dark purple crude product. This crude product was purified by chromatography, to obtain a yellowish white solid. This yellowish white solid was washed with a mixed solvent of 10% ethyl acetate/diethyl ether, and thus the title compound (6.1 g) was obtained as a white solid.

¹H NMR (300 MHz, DMSO-d₆) δ ppm 2.67 (2H, t, J=6.6 Hz), 3.34 (2H, td, J=6.6, 5.8 Hz), 4.10 (2H, s), 8.56 (1H, br. s.).

### Reference Example 41

### 2-Chloro-N-(2-cyanoethyl)-N-methylacetamide

3-(Methylamino)propanenitrile (39.6 g) and triethylamine (57.2 g) were dissolved in tetrahydrofuran (300 ml), and chloroacetyl chloride (53.2 g) dissolved in tetrahydrofuran (50 ml) was added to the solution under ice cooling. The mixture was stirred overnight at room temperature. White precipitates generated therefrom were filtered, and the filtrate was concentrated under reduced pressure. The resulting residue was purified by chromatography, and the title compound (63.6 g) was obtained as a brown oily substance.

¹H NMR (400 MHz, DMSO-d₆) δ ppm 2.73 (1.34H, t, J=6.6 Hz), 2.86 (0.66H, t, J=6.8 Hz), 2.87 (1H, s), 3.05 (2H, s), 3.55 (1.34H, t, J=6.6 Hz), 3.64 (0.66H, t, J=6.8 Hz), 4.42 (1.34H, s), 4.45 (0.66H, s).

### Reference Example 42

### 2-Chloro-N-(2-cyanoethyl)propanamide

3-Aminopropanenitrile (700 mg) and triethylamine (2.1 ml) were dissolved in tetrahydrofuran (10 ml), and 2-chloropropanoyl chloride (1.4 g) dissolved in tetrahydrofuran (5 ml) was added to the solution under ice cooling. The mixture was stirred for one hour at room temperature. Diethyl ether (20 ml) was added to the reaction solution, and generated white precipitates were filtered. The filtrate was concentrated under reduced pressure, and the resulting residue was purified by chromatography, and thus the title compound (1.36 g) was obtained as a yellowish white solid.

¹H NMR (400 MHz, DMSO-d₆) δ ppm 1.53 (3H, d, J=6.8 Hz), 2.68 (2H, t, J=6.5 Hz), 3.33 (2H, td, J=6.5, 5.8 Hz), 4.51 (1H, q, J=6.8 Hz), 8.61 (1H, t, J=5.8 Hz).

### Reference Example 43

### 1-Chloro-N-(2-cyanoethyl)methanesulfonamide

3-Aminopropanenitrile (700 mg) and triethylamine (2.0 ml) were dissolved in tetrahydrofuran (20 ml), and chloromethanesulfonyl chloride (1.49 g) dissolved in tetrahydrofuran (10 ml) was added dropwise to the solution over 5 minutes under ice cooling. Subsequently, the mixture was stirred for one hour at room temperature. Ethyl acetate (50 ml) was added to the reaction solution, and generated white precipitates were filtered. The filtrate was concentrated under reduced pressure, and the resulting residue was dissolved in ethyl acetate (150 ml). The solution was washed with a saturated aqueous solution of sodium hydrogen carbonate and saturated brine, dried over anhydrous magnesium sulfate, and concentrated under reduced pressure, to obtain a colorless oily residue. This oily residue was purified by chromatography, and thus the title compound (1.32 g) was obtained as a white solid.

¹H NMR (400 MHz, DMSO-d₆) δ ppm 2.69 (2H, t, J=6.6 Hz), 3.27 (2H, t, J=6.6 Hz), 4.99 (2H, s), 8.16 (1H, s).

### Reference Example 44

### N-(2-bromoethyl)-3-cyanopropanamide

3-Cyanopropanoic acid (500 mg) was dissolved in N,N-dimethylformamide (5 ml), and 1-ethyl-3-(3-dimethylaminopropyl)carbodiimide hydrochloride (960 mg), 1-hydroxybenzotriazole (675 mg), 2-bromoethylamine hydrobromide (1.0 g) and triethylamine (0.7 ml) were added sequentially to the solution. The mixture was stirred overnight at room temperature. Subsequently, the reaction solution was diluted with ethyl acetate (100 ml), and the dilution was washed sequentially with water, a saturated aqueous solution of sodium hydrogen carbonate and saturated brine, dried over anhydrous magnesium sulfate, and then concentrated under reduced pressure, to obtain a light yellow crude product. This crude product was purified by chromatography, and thus the title compound (473 mg) was obtained as a white solid.

¹H NMR (400 MHz, DMSO-d₆) δ ppm 2.46 (2H, t, J=7.0 Hz), 2.64 (2H, t, J=7.0 Hz), 3.40 (1.3H, td, J=6.1, 5.7 Hz), 3.45-3.49 (1.4H, m), 3.61 (1.3H, t, J=6.1 Hz), 8.27-8.39 (1H, m).

### Reference Example 45

### 3-Chloro-N-(2-cyanoethyl)propane-1-sulfonamide

3-Aminopropanenitrile (700 mg) and triethylamine (2.1 ml) were dissolved in tetrahydrofuran (20 ml), and 3-chloropropane-1-sulfonyl chloride (1.95 g) dissolved in tetrahydrofuran (10 ml) was added dropwise to the solution over 5 minutes under ice cooling. Subsequently, the mixture was stirred for 2 hours at room temperature. Diethyl ether (20 ml) was added to the reaction solution, and white precipitates generated therefrom were filtered. The filtrate was concentrated under reduced pressure, and the resulting residue was purified by chromatography, and thus the title compound (1.83 g) was obtained as white crystals.

¹H NMR (400 MHz, DMSO-d₆) δ ppm 2.04-2.16 (2H, m), 2.67 (2H, t, J=6.5 Hz), 3.13-3.25 (4H, m), 3.74 (2H, t, J=6.6 Hz), 7.64 (1H, br. s.).

### Reference Example 46

### 4-Bromo-N-(cyanomethyl)butanamide

4-Bromobutanoic acid (1.83 g) was dissolved in N,N-dimethylformamide (10 ml), and 1-ethyl-3-(3-dimethylaminopropyl)carbodiimide hydrochloride (2.3 g), 1-hydroxybenzotriazole (1.35 g), 2-aminoacetonitrile hydrochloride (930 mg) and triethylamine (1.4 ml) were added sequentially to the solution. The mixture was stirred overnight at room temperature. Subsequently, the reaction solution was diluted with ethyl acetate (200 ml), and the dilution was washed with water, a saturated aqueous solution of sodium hydrogen carbonate and saturated brine sequentially, dried over anhydrous magnesium sulfate, and then concentrated under reduced pressure, to obtain a light yellow crude product. This crude product was purified by chromatography, and thus the title compound (882 mg) was obtained as a white solid.

¹H NMR (300 MHz, DMSO-d₆) δ ppm 1.96 (2H, tt, J=7.2, 6.6 Hz), 2.31 (2H, t, J=7.2 Hz), 3.64 (2H, t, J=6.6 Hz), 4.11 (2H, d, J=5.6 Hz), 8.71 (1H, t, J=5.6 Hz).

### Reference Example 47

### N-but-3-yn-1-yl-2-chloroacetamide

### Reference Example 47a)

3-Butynyl 4-toluenesulfonate (2.24 g) and sodium azide (1.95 g) were introduced into N,N-dimethylformamide (20 ml), and the resulting mixture was stirred for 2 hours at 80° C. Subsequently, water (20 ml) was added to the reaction mixture liquid, and the resulting mixture was extracted with diethyl ether. The resultant was washed with water and saturated brine, dried over anhydrous magnesium sulfate, and then concentrated under reduced pressure, to obtain a colorless oily substance (627 mg). This colorless oily substance was dissolved in diethyl ether (5 ml), and the solution was slowly added dropwise to a suspension of lithium aluminum hydride (1.0 g) in diethyl ether (20 ml) under ice cooling. The mixture was stirred for 1.5 hours. Subsequently, water (1 ml), a 3 M aqueous solution of sodium hydroxide (1 ml), water (3 ml) and diethyl ether (20 ml) were added sequentially to the reaction solution under ice cooling, and the resulting mixture was stirred for 10 minutes at room temperature. Subsequently, precipitates were filtered, and a 4 M hydrogen chloride/ethyl acetate solution (2 ml) was added to the filtrate. The mixture was concentrated under reduced pressure to obtain a white solid. A mixed solvent of ethanol/toluene was added to this white solid, and the mixture was concentrated under reduced pressure. Then, the mixture was washed with a mixed solvent of 10% ethanol/hexane, and thus but-3-yn-1-amine hydrochloride (389 mg) was obtained as a white solid.

¹H NMR (400 MHz, DMSO-d₆) δ ppm 2.54 (2H, td, J=7.5, 2.7 Hz), 2.91 (2H, t, J=7.5 Hz), 3.06 (1H, t, J=2.7 Hz), 8.25 (3H, br. s.).

### Reference Example 47b)

Chloroacetyl chloride (340 mg) dissolved in tetrahydrofuran (3 ml) was added under ice cooling to a mixture of but-3-yn-1-amine hydrochloride (318 mg) obtained in Reference Example (47a), triethylamine (0.63 ml) and tetrahydrofuran (10 ml). The mixture was stirred for one hour at room temperature, and then diethyl ether (10 ml) was added to the reaction mixture liquid. Precipitates were filtered, and the filtrate was concentrated under reduced pressure, to obtain a dark green crude product. This crude product was purified by chromatography, and the title compound, N-but-3-yn-1-yl-2-chloroacetamide (231 mg), was obtained as a brown oily substance.

¹H NMR (400 MHz, DMSO-d₆) δ ppm 2.32 (2H, td, J=7.1, 2.7 Hz), 2.86 (1H, t, J=2.7 Hz), 3.21 (2H, td, J=7.1, 5.9 Hz), 4.07 (2H, s), 8.38 (1H, t, J=5.9 Hz).

### Reference Example 48

### 5-Bromo-N-(2-cyanoethyl)pentanamide

3-Aminopropanenitrile (700 mg) and triethylamine (1.79 ml) were dissolved in tetrahydrofuran (20 ml), and 5-bromopentanoyl chloride (1.99 g) dissolved in tetrahydrofuran (10 ml) was added to the solution dropwise under ice cooling. The mixture was stirred for 2 hours at room temperature. Subsequently, diethyl ether (20 ml) was added to the reaction solution, and precipitates generated therefrom were filtered, and the filtrate was concentrated under reduced pressure, to obtain a yellowish white crude product. This crude product was purified by chromatography, and thus the title compound (2.22 g) was obtained as a white solid.

¹H NMR (300 MHz, DMSO-d₆) δ ppm 1.57-1.68 (2H, m), 1.73-1.86 (2H, m), 2.13 (2H, t, J=7.3 Hz), 2.63 (2H, t, J=6.5 Hz), 3.27 (2H, td, J=6.5, 5.6 Hz), 3.53 (2H, t, J=6.6 Hz), 8.21 (1H, t, J=5.6 Hz).

### Reference Example 49

### 4-Bromo-N-(2-cyanoethyl)butanamide

3-Aminopropanenitrile (1.54 g) and triethylamine (3.5 ml) were dissolved in tetrahydrofuran (50 ml), and 4-bromobutanoyl chloride (3.8 g) dissolved in tetrahydrofuran (10 ml) was added dropwise to the solution over 5 minutes under ice cooling. The mixture was stirred overnight at room temperature. Subsequently, ethyl acetate (200 ml) was added to the reaction solution, and precipitates generated therefrom were filtered. The filtrate was washed with water, 1 M hydrochloric acid, a saturated aqueous solution of sodium hydrogen carbonate and saturated brine, dried over anhydrous magnesium sulfate, and then concentrated under reduced pressure, to obtain a yellow crude product. This crude product was purified by chromatography, and thus the title compound (2.2 g) was obtained as a white solid.

¹H NMR (300 MHz, DMSO-d₆) δ ppm 2.02 (2H, tt, J=7.3, 6.6 Hz), 2.26 (2H, t, J=7.3 Hz), 2.64 (2H, t, J=6.5 Hz), 3.28 (2H, td, J=6.5, 5.5 Hz), 3.53 (2H, t, J=6.6 Hz), 8.29 (1H, t, J=5.5 Hz).

### Reference Example 50

### Ethyl

5-oxo-2-{[1-{[4-(2,2,2-trifluoroethoxy)phenyl]amino]butylidene]amino}-4,5-dihydro-1H-pyrrol-3-carboxylate

### Reference Example 50a)

A mixture of ethyl 2-amino-5-oxo-4,5-dihydro-1H-pyrrol-3-carboxylate (0.340 g) obtained in Reference Example 5, and 1,1,1-trimethoxybutane (5 ml) was stirred overnight at 100° C., and then was concentrated under reduced pressure. The residue was purified by chromatography, and thus ethyl 2-[(1-methoxybutylidene)amino]-5-oxo-4,5-dihydro-1H-pyrrol-3-carboxylate (0.405 g) was obtained as a yellow oily substance.

¹H NMR (300 MHz, DMSO-d₆) δ ppm 0.84 (3H, t, J=7.4 Hz), 1.13 (3H, t, J=7.0 Hz), 1.44-1.59 (2H, m), 2.28 (2H, t, J=7.4 Hz), 3.20 (2H, s), 3.74 (3H, s), 3.97 (2H, q, J=7.0 Hz), 10.33 (1H, s).

### Reference Example 50b)

A mixture of ethyl 2-[(1-methoxybutylidene)amino]-5-oxo-4,5-dihydro-1H-pyrrol-3-carboxylate (0.386 g) obtained in Reference Example (50a), 4-(2,2,2-trifluoroethoxy)aniline (0.290 g) and toluene (50 ml) was heated to reflux overnight, and then was concentrated under reduced pressure. The residue was purified by chromatography, and thus the title compound, ethyl 5-oxo-2-{[1-{[4-(2,2,2-trifluoroethoxy)phenyl]amino}butylidene]amino}-4,5-dihydro-1H-pyrrol-3-carboxylate (0.458 g), was obtained as a white solid.

¹H NMR (300 MHz, DMSO-d₆) δ ppm 0.89 (3H, t, J=7.2 Hz), 1.10 (3H, t, J=7.1 Hz), 1.47-1.68 (2H, m), 2.38 (2H, t, J=7.3 Hz), 3.16 (2H, s), 3.95 (2H, q, J=7.2 Hz), 4.70 (2H, q, J=9.0 Hz), 7.00 (2H, d, J=8.9 Hz), 7.56 (2H, d, J=8.9 Hz), 9.44 (1H, s), 10.13 (1H, s).

### Reference Example 51

### 1-Isothiocyanato-4-(2,2,3,3,3-pentafluoropropoxy)benzene

To a mixture of a tetrahydrofuran (50 ml) solution of 4-(2,2,3,3,3-pentafluoropropoxy)aniline (7.24 g) obtained by the method of Reference Example 30, or a method pursuant to thereto, and an aqueous solution (50 ml) of sodium carbonate (3.18 g) in an ice water bath, thiocarbonyl dichloride (3.50 g) was added dropwise over 5 minutes. The resulting mixture was stirred for 30 minutes at room temperature, and then was extracted with ethyl acetate. The organic layer was washed with saturated brine, dried over anhydrous sodium sulfate, and then concentrated under reduced pressure. The residue was suspended in a mixed solvent of ethyl acetate/hexane, and the solid was collected by filtration. The solid was washed with hexane and then dried, and thus the title compound (4.82 g) was obtained as a white solid.

¹H NMR (300 MHz, CHLOROFORM-d) δ ppm 4.41 (2H, tq, J=12.1, 0.81 Hz), 6.91 (2H, d, J=8.9 Hz), 7.20 (2H, d, J=8.9 Hz).

### Reference Example 52

### 1-(Cyclopropylmethoxy)-4-isothiocyanatobenzene

Sodium carbonate (530 mg) was dissolved in water (25 ml), and the solution was added to a tetrahydrofuran solution (25 ml) of 4-(cyclopropylmethoxy)aniline (998 mg) obtained by the method of Reference Example 13, or a method pursuant to thereto. To this mixed solution, thiocarbonyl dichloride (632 mg, 0.42 ml) dissolved in tetrahydrofuran (5 ml) was added dropwise over 3 minutes. The reaction solution was stirred for 30 minutes at room temperature, and then was extracted with ethyl acetate. The resultant was washed with saturated brine, dried over anhydrous magnesium sulfate, and then concentrated under reduced pressure, to obtain a brown crude product. This crude product was purified by chromatography, and thus the title compound (955 mg) was obtained as white needle-shaped crystals.

¹H NMR (300 MHz, DMSO-d₆) δ ppm 0.25-0.37 (2H, m), 0.50-0.64 (2H, m), 1.10-1.32 (1H, m), 3.83 (2H, d, J=7.2 Hz), 6.97 (2H, d, J=9.1 Hz), 7.37 (2H, d, J=9.1 Hz).

### Reference Example 53

### Ethyl(tetrahydro-2H-pyran-4-yloxy)acetate

Lithium aluminum hydride (950 mg) was suspended in a mixed solvent of diethyl ether (50 ml) and tetrahydrofuran (50 ml), and a solution of tetrahydro-4H-pyran-4-one (5.00 g) in diethyl ether (10 ml) was added dropwise to the suspension under ice cooling. The mixture was stirred for 2 hours under ice cooling, and then water (1 ml), a 6 M aqueous solution of sodium hydroxide (0.75 ml) and water (1 ml) were added thereto. The mixture was stirred for 30 minutes under ice cooling, and then precipitates generated therefrom were filtered off. The resulting filtrate was concentrated under reduced pressure. The resulting residue was dissolved in dichloromethane (100 ml), and a rhodium acetate dimer (221 mg) was added to the solution. To this mixture, a solution of ethyl diazoacetate (6.28 g) in dichloromethane (10 ml) was added dropwise at room temperature, and the resulting mixture was stirred for 15 hours at room temperature under a nitrogen atmosphere. Ethanol was added to the reaction mixture, and precipitates generated therefrom were filtered off. The resulting filtrate was concentrated under reduced pressure. The resulting residue was purified by chromatography, and thus the title compound (6.41 g) was obtained as a colorless oily substance.

¹H NMR (300 MHz, CHLOROFORM-d) δ ppm 1.29 (3H, t, J=7.1 Hz), 1.58-1.72 (2H, m), 1.88-1.99(2H, m), 3.39-3.48 (2H, m), 3.55-3.65 (1H, m), 3.96 (2H, dt, J=11.9, 4.3 Hz), 4.13 (2H, s), 4.23 (2H, q, J=7.1 Hz).

### Reference Example 54

### 2-(Tetrahydro-2H-pyran-4-yloxy)ethanol

Lithium aluminum hydride (0.5 g) was suspended in tetrahydrofuran (50 ml), and a tetrahydrofuran (10 ml) solution of ethyl (tetrahydro-2H-pyran-4-yloxy)acetate (2.5 g) obtained in Reference Example 53 was added dropwise under ice cooling. The mixture was stirred for 2 hours under ice cooling, and then water (0.5 ml), a 5 M aqueous solution of sodium hydroxide (0.5 ml) and water (0.5 ml) were added thereto. The mixture was stirred for 30 minutes at room temperature. Then, precipitates generated therefrom were filtered off. The resulting filtrate was concentrated under reduced pressure, and thus the title compound (1.81 g) was obtained as a colorless oily substance.

¹H NMR (300 MHz, CHLOROFORM-d) δ ppm 1.53-1.66 (2H, m), 1.87-1.97 (2H, m), 2.16 (1H, t, J=6.1 Hz), 3.40-3.49 (2H, m), 3.49-3.57 (1H, m), 3.57-3.61 (2H, m), 3.71-3.79 (2H, m), 3.95 (2H, dt, J=11.9, 4.3 Hz).

### Reference Example 55

### 2-(Tetrahydro-2H-pyran-4-yloxy)ethyl 4-methylbenzenesulfonate

To a mixture of 2-(tetrahydro-2H-pyran-4-yloxy)ethanol (1.7 g) obtained in Reference Example 54, N,N-dimethylpyridine-4-amine (several mg), triethylamine (1.78 ml) and tetrahydrofuran (30 ml), 4-methylbenzenesulfonyl chloride (2.43 g) was added under ice cooling, and the resulting mixture was stirred for 3 days. Precipitates were filtered off, and the resulting filtrate was diluted with water, and the dilution was extracted with ethyl acetate. The organic layer was washed with 1 M hydrochloric acid, water and saturated brine, dried over anhydrous magnesium sulfate, and then concentrated under reduced pressure. The resulting residue was purified by chromatography, and thus the title compound (1.40 g) was obtained as a colorless oily substance.

¹H NMR (300 MHz, CHLOROFORM-d) δ ppm 1.43-1.57 (2H, m), 1.76-1.86 (2H, m), 2.45 (3H, s), 3.31-3.53 (3H, m), 3.64-3.69 (2H, m), 3.88 (2H, dt, J=11.8, 4.5 Hz), 4.14-4.18 (2H, m), 7.34 (2H, d, J=8.1 Hz), 7.81 (2H, d, J=8.1 Hz).

### Reference Example 56

### 2-(4-Hydroxytetrahydro-2H-pyran-4-yl)ethyl 4-methylbenzenesulfonate

Lithium aluminum hydride (505 mg) was suspended in tetrahydrofuran (30 ml), and a tetrahydrofuran (10 ml) solution of ethyl(4-hydroxytetrahydro-2H-pyran-4-yl)acetate (2.5 g) obtained by a method described in a published document, WO 05/105802, or a method pursuant to thereto, was added dropwise to the suspension under ice cooling. The mixture was stirred for 2 hours under ice cooling, and then water (0.5 ml), a 5 M aqueous solution of sodium hydroxide (0.5 ml) and water (0.5 ml) were added thereto. The mixture was stirred for 30 minutes at room temperature. Then, precipitates generated therefrom were filtered off. The resulting filtrate was concentrated under reduced pressure, to obtain a colorless oily substance was obtained. To a mixture of the obtained oily substance, N,N-dimethylpyridine-4-amine (several mg), triethylamine (1.85 ml) and tetrahydrofuran (30 ml), 4-methylbenzenesulfonyl chloride (2.54 g) was added under ice cooling, and the resulting mixture was stirred for 15 hours at room temperature. The reaction mixture was diluted with water, and the dilution was extracted with ethyl acetate. The organic layer was washed with 1 M hydrochloric acid, water and saturated brine, dried over anhydrous magnesium sulfate, and then concentrated under reduced pressure. The resulting residue was purified by chromatography, and thus the title compound (2.10 g) was obtained as a colorless oily substance.

¹H NMR (300 MHz, CHLOROFORM-d) δ ppm 1.44-1.74 (4H, m), 1.87 (2H, t, J=6.4 Hz), 2.46 (3H, s), 3.64-3.79 (4H, m), 4.25 (2H, t, J=6.4 Hz), 7.36 (2H, d, J=8.1 Hz), 7.79 (2H, d, J=8.1 Hz).

### Reference Example 57

### Tert-butyl 3-(tetrahydro-2H-pyran-4-yloxy)propanoate

To a residue obtained by concentrating benzyltrimethylammonium hydroxide (40% methanol solution, 0.5 ml) under reduced pressure, tetrahydro-2H-pyran-4-ol (1.02 g) and tert-butyl acrylate (1.41 g) were added, and the resulting mixture was stirred for 3 days at 50° C. Then, the mixture was purified by chromatography, and thus the title compound (1.33 g) was obtained as a colorless oily substance.

¹H NMR (300 MHz, CHLOROFORM-d) δ ppm 1.46 (9H, s), 1.51-1.65 (2H, m), 1.83-1.94 (2H, m), 2.48 (2H, t, J=6.3 Hz), 3.39-3.55 (3H, m), 3.70 (2H, t, J=6.3 Hz), 3.88-3.97 (2H, m).

### Reference Example 58

### 3-(Tetrahydro-2H-pyran-4-yloxy)propan-1-ol

Lithium aluminum hydride (0.76 g) was suspended in tetrahydrofuran (30 ml), and a tetrahydrofuran (50 ml) solution of tert-butyl 3-(tetrahydro-2H-pyran-4-yloxy)propanoate (4.6 g) obtained in Reference Example 57 was added dropwise to the suspension at −40° C. The mixture was returned to room temperature and stirred for 2 hours, and then water (0.76 ml), a 5 M aqueous solution of sodium hydroxide (0.76 ml) and water (0.76 ml) were added thereto. Precipitates generated therefrom were filtered off, and the resulting filtrate was concentrated under reduced pressure, and thus the title compound (2.81 g) was obtained as a colorless oily substance.

¹H NMR (300 MHz, CHLOROFORM-d) δ ppm 1.50-1.67 (2H, m), 1.79-1.97 (4H, m), 2.41 (1H, t, J=5.4 Hz), 3.38-3.56 (3H, m), 3.67 (2H, t, J=5.7 Hz), 3.74-3.83 (2H, m), 3.93 (2H, dt, J=11.8, 4.4 Hz).

### Reference Example 59

### 3-(Tetrahydro-2H-pyran-4-yloxy)propyl 4-methylbenzenesulfonate

To a mixture of 3-(tetrahydro-2H-pyran-4-yloxy)propan-1-ol (2.80 g) obtained in Reference Example 58, N,N,N′,N′-tetramethylhexan-1,6-diamine (360 mg), triethylamine (4.85 ml) and toluene (30 ml), a solution of 4-methylbenzenesulfonyl chloride (3.98 g) in toluene (80 ml) was added under ice cooling, and the resulting mixture was stirred for one week at room temperature. The mixture was diluted with water, and the dilution was extracted with ethyl acetate. The organic layer was washed with saturated brine, dried over anhydrous magnesium sulfate, and then concentrated under reduced pressure. The resulting residue was purified by chromatography, and thus the title compound (4.25 g) was obtained as a colorless oily substance.

¹H NMR (300 MHz, CHLOROFORM-d) δ ppm 1.39-1.53 (2H, m), 1.73-1.83 (2H, m), 1.85-1.95 (2H, m), 2.45 (3H, s), 3.29-3.45 (3H, m), 3.48 (2H, t, J=6.1 Hz), 3.87 (2H, dt, J=11.7, 4.5 Hz), 4.15 (2H, t, J=6.1 Hz), 7.35 (2H, d, J=8.1 Hz), 7.80 (2H, d, J=8.1 Hz).

### Reference Example 60

### 3-[(1-Methylethypsulfanyl]propan-1-ol

To a mixture of 3-sulfanylpropan-1-ol (5.0 g), 2-iodopropane (5.96 ml) and methanol (60 ml), a 1 M aqueous solution of sodium hydroxide (60 ml) was added dropwise under ice cooling, and then the mixture was stirred for 15 hours at room temperature. Methanol was distilled off under reduced pressure, and then the resultant was extracted with diethyl ether. The obtained organic layer was washed with saturated brine, dried over anhydrous magnesium sulfate, and then concentrated under reduced pressure. The resulting residue was purified by chromatography, and thus the title compound (6.24 g) was obtained as a colorless oily substance.

¹H NMR (300 MHz, CHLOROFORM-d) δ ppm 1.28 (6H, d, J=6.8 Hz), 1.79-1.91 (2H, m), 2.66 (2H, t, J=7.1 Hz), 2.87-3.01 (1H, m), 3.76 (2H, t, J=5.8 Hz).

### Reference Example 61

### 3-[(1-Methylethyl)sulfanyl]propyl 4-methylbenzenesulfonate

To a mixture of 3-[(1-methylethyl)sulfanyl]propan-1-ol (2.68 g) obtained in Reference Example 60, N,N-dimethylpyridine-4-amine (several mg) and pyridine (20 ml), 4-methylbenzenesulfonyl chloride (2.93 g) was added under ice cooling, and the resulting mixture was stirred for 15 hours at room temperature. The solvent was distilled off under reduced pressure, and then the resultant was diluted with ethyl acetate. The dilution was washed with 1 M hydrochloric acid, a saturated aqueous solution of sodium hydrogen carbonate and saturated brine, dried over anhydrous magnesium sulfate, and then concentrated under reduced pressure. The resulting residue was purified by chromatography, and thus the title compound (4.45 g) was obtained as a colorless oily substance.

¹H NMR (300 MHz, CHLOROFORM-d) δ ppm 1.21 (6H, d, J=6.8 Hz), 1.85-1.96 (2H, m), 2.45 (3H, s), 2.54 (2H, t, J=7.2 Hz), 2.76-2.88 (1H, m), 4.14 (2H, t, J=6.1 Hz), 7.35 (2H, d, J=8.1 Hz), 7.80 (2H, d, J=8.1 Hz).

### Reference Example 62

### 3-[(1-Methylethyl)sulfonyl]propyl 4-methylbenzenesulfonate

To a methanol (80 ml) solution of 3-[(1-methylethyl)sulfanyl]propyl 4-methylbenzenesulfonate (2.88 g) obtained in Reference Example 61, an aqueous solution (80 ml) of Oxone (registered trademark) monopersulfate compound (15.4 g) was added dropwise at room temperature. The mixture was stirred for 6 hours at room temperature, and then methanol was distilled off under reduced pressure. The resulting aqueous solution was extracted with ethyl acetate. The organic layer was washed with saturated brine, dried over anhydrous magnesium sulfate, and then concentrated under reduced pressure. Thus, the title compound (2.51 g) was obtained as a white powder.

¹H NMR (300 MHz, CHLOROFORM-d) δ ppm 1.39 (6H, d, J=6.8 Hz), 2.15-2.29 (2H, m), 2.46 (3H, s), 2.94-3.14 (3H, m), 4.18 (2H, t, J=5.9 Hz), 7.37 (2H, d, J=8.1 Hz), 7.79 (2H, d, J=8.1 Hz).

### Reference Example 63

### 3-[(Cyclopropylmethypsulfanyl]propan-1-ol

To a mixture of 3-sulfanylpropan-1-ol (5.0 g), (bromomethyl)cyclopropane (8.06 g) and methanol (60 ml), a 1 M aqueous solution of sodium hydroxide (60 ml) was added dropwise under ice cooling, and then the mixture was stirred for 15 hours at room temperature. Methanol was distilled off under reduced pressure, and then the resultant was extracted with diethyl ether. The resulting organic layer was washed with saturated brine, dried over anhydrous magnesium sulfate, and then concentrated under reduced pressure. The resulting residue was purified by chromatography, and thus the title compound (5.45 g) was obtained as a colorless oily substance.

¹ H NMR (300 MHz, CHLOROFORM-d) δ ppm 0.17-0.25 (2H, m), 0.53-0.62 (2H, m), 0.91-1.07 (1H, m), 1.77-1.93 (3H, m), 2.48 (2H, d, J=7.2 Hz), 2.72 (2H, t, J=7.0 Hz), 3.71-3.82 (2H, m).

### Reference Example 64

### 3-[(Cyclopropylmethyl)sulfanyl]propyl 4-methylbenzenesulfonate

To a mixture of 3-[(cyclopropylmethyl)sulfanyl]propan-1-ol (2.93 g) obtained in Reference Example 63, N,N-dimethylpyridine-4-amine (several mg) and pyridine (20 ml), 4-methylbenzenesulfonyl chloride (2.93 g) was added under ice cooling, and the resulting mixture was stirred for 15 hours at room temperature. The solvent was distilled off under reduced pressure, and then the resultant was diluted with ethyl acetate. The dilution was washed with 1 M hydrochloric acid, a saturated aqueous solution of sodium hydrogen carbonate and saturated brine, dried over anhydrous magnesium sulfate, and then concentrated under reduced pressure. The resulting residue was purified by chromatography, and thus the title compound (4.05 g) was obtained as a colorless oily substance.

¹H NMR (300 MHz, CHLOROFORM-d) δ ppm 0.13-0.20 (2H, m), 0.50-0.59 (2H, m), 0.82-1.01 (1H, m), 1.86-1.98 (2H, m), 2.38 (2H, d, J=7.0 Hz), 2.45 (3H, s), 2.59 (2H, t, J=7.1 Hz), 4.14 (2H, t, J=6.1 Hz), 7.35 (2H, d, J=8.4 Hz), 7.79 (2H, d, J=8.4 Hz).

### Reference Example 65

### 3-[(Cyclopropylmethyl)sulfonyl]propyl 4-methylbenzenesulfonate

To a methanol (60 ml) solution of 3-[(cyclopropylmethyl)sulfanyl]propyl 4-methylbenzenesulfonate (2.0 g) obtained in Reference Example 64, an aqueous solution (60 ml) of Oxone (registered trademark) monopersulfate compound (9.25 g) was added dropwise at room temperature. The mixture was stirred for 6 hours at room temperature, and then methanol was distilled off under reduced pressure. The resulting aqueous solution was extracted with ethyl acetate. The organic layer was washed with saturated brine, dried over anhydrous magnesium sulfate, and then concentrated under reduced pressure. Thus, the title compound (2.01 g) was obtained as a colorless oily substance.

¹H NMR (300 MHz, CHLOROFORM-d) δ ppm 0.35-0.43 (2H, m), 0.73-0.81 (2H, m), 1.08-1.21 (1H, m), 2.17-2.28 (2H, m), 2.46 (3H, s), 2.90 (2H, d, J=7.2 Hz), 3.06-3.14 (2H, m), 4.18 (2H, t, J=5.9 Hz), 7.37 (2H, d, J=8.1 Hz), 7.79 (2H, d, J=8.1 Hz).

### Reference Example 66

### 4-(Oxiran-2-ylmethoxy)tetrahydro-2H-pyran

To a mixture of tetrahydro-2H-pyran-4-ol (4.60 g), N,N,N,N-tetrabutylammonium bromide (1.45 g), sodium hydroxide (9.0 g), water (9 ml) and toluene (25 ml), a solution of 2-(chloromethyl)oxirane (10.5 g) in toluene (10 ml) was added dropwise at 60° C., and the resulting mixture was stirred for 8 hours at 60° C. The reaction mixture was returned to room temperature, and then the organic layer was collected by partition and was diluted with diethyl ether. The dilution was washed with water and saturated brine, dried over anhydrous magnesium sulfate, and then concentrated under reduced pressure. The resulting residue was purified by chromatography, and thus the title compound (4.49 g) was obtained as a colorless oily substance.

¹H NMR (300 MHz, CHLOROFORM-d) δ ppm 1.50-1.70 (2H, m), 1.83-1.98 (2H, m), 2.63 (1H, dd, J=5.0, 2.9 Hz), 2.81 (1H, dd, J=5.0, 4.2 Hz), 3.10-3.20 (1H, m), 3.37-3.50 (3H, m), 3.51-3.64 (1H, m), 3.76 (1H, dd, J=11.6, 2.9 Hz), 3.95 (2H, dt, J=11.6, 4.2 Hz).

### Reference Example 67

### 1-Isothiocyanato-4-(2,2,2-trifluoroethoxy)benzene

4-(2,2,2-Trifluoroethoxy)aniline (10 g) was dissolved in tetrahydrofuran (100 ml), and 6 M hydrochloric acid (9 ml) was added thereto. Then, the mixture was cooled to −5° C. To the mixture was added dropwise a solution of thiophosgene (4.01 ml) in tetrahydrofuran (20 ml) over 5 minutes. After stirring at −5° C. for 10 minutes, a saturated aqueous solution of sodium hydrogen carbonate (125 ml) was added to the reaction mixture and the mixture was extracted with ethyl acetate two times (200 ml and 100 ml). The extract was washed with saturated brine, dried over anhydrous magnesium sulfate, and evaporated under reduced pressure to give a brown residue. The residue was purified by chromatography to give a pale yellow solid, which was washed with hexane to give the title compound (10.7 g) as white crystals.

¹H NMR (300 MHz, DMSO-d₆) δ ppm 4.81 (2H, q, J=8.8 Hz), 7.13 (2H, d, J=9.1 Hz), 7.45 (2H, d, J=9.1 Hz).

### Reference Example 68

### 4-(2,2,2-Trifluoroethoxy)aniline

1-Nitro-4-(2,2,2-trifluoroethoxy)benzene (2.21 g) obtained by the method of Reference Example (6a), or a method pursuant to thereto was dissolved in ethanol (50 ml) and to the solution was added water (5 ml), reduced iron (2.79 g) and calcium chloride (0.56 g). The mixture was heated to reflux for 18 hours, then cooled to room temperature. The insolubles were filtered off, washed with methanol, and the filtrate was concentrated under reduced pressure. The residue was dissolved in ethyl acetate (200 ml) and washed with saturated brine, dried over anhydrous magnesium sulfate, and concentrated under reduced pressure to give an orange-colored residue. The residue was purified by chromatography to give the title compound (1.89 g) as a brown solid.

¹H NMR (400 MHz, DMSO-d₆) δ ppm 4.53 (2H, q, J=9.0 Hz), 4.77(2H, s), 6.52 (2H, d, J=8.8 Hz), 6.75 (2H, d, J=8.8 Hz).

### Example 1

### 3-(4-Ethoxyphenyl)-2-thioxo-1,2,3,5-tetrahydro-4H-pyrrolo[3,2-d]pyrimidin-4-one

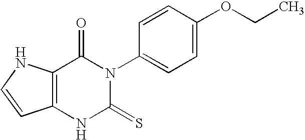

Toluene was added to a mixture of ethyl 3-amino-1H-pyrrol-2-carboxylate (1.78 g) obtained by a method described in a published document, Journal of Organic Chemistry (J. Org. Chem.), Vol. 64, p. 8411 (1999), or a method pursuant to thereto, 1-isothiocyanato-4-ethoxybenzene (2.07 g) and 4-dimethylaminopyridine (140 mg), and the mixture was concentrated under reduced pressure, and then was dissolved in N,N-dimethylformamide (23 ml). Sodium hydride (60% in oil, 1.52 g) was added thereto in several divided portions under ice cooling, and the resulting mixture was stirred for 30 minutes. Then, the mixture was stirred for 1.5 hours at room temperature. Subsequently, the reaction solution was poured into ice water (50 ml), and 1 M hydrochloric acid was added thereto to acidify the reaction solution. A solid precipitated therefrom was collected by filtration, washed sequentially with water and diethyl ether, and then dissolved in tetrahydrofuran (150 ml). The solution was diluted with ethyl acetate (300 ml), and then the dilution was washed with saturated brine, dried over anhydrous magnesium sulfate, and then concentrated under reduced pressure. A yellow solid generated therefrom was washed with diethyl ether, and thus the title compound (2.82 g) was obtained as a yellowish white solid.

¹H NMR (400 MHz, DMSO-d₆) δ ppm 1.36 (3H, t, J=7.0 Hz), 4.07 (2H, q, J=7.0 Hz), 6.01 (1H, dd, J=2.7, 2.1 Hz), 6.96 (2H, d, J=8.8 Hz), 7.08 (2H, d, J=8.8 Hz), 7.34 (1H, dd, J=3.2, 2.7 Hz), 12.30 (1H, br. s.), 12.91 (1H, s).

### Example 2

### 2-Thioxo-3-[4-(2,2,2-trifluoroethoxy)phenyl]-1,2,3,5-tetrahydro-4H-pyrrolo[3,2-d]pyrimidin-4-one

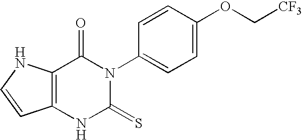

Ethyl 3-isothiocyanato-1H-pyrrol-2-carboxylate (1.96 g) obtained by the method of Reference Example 31, or a method pursuant to thereto, and 4-(2,2,2-trifluoroethoxy)aniline (1.91 g) were added to acetonitrile (50 ml), and the mixture was heated to reflux for 2 hours. The reaction solution was returned to room temperature, and then was concentrated under reduced pressure and dissolved in ethanol (50 ml). To this ethanol solution, an ethanol (10 ml) solution of potassium tert-butoxide (3.3 g) was added, and the mixture was heated to reflux for 30 minutes. The reaction mixture was returned to room temperature. Then, ethanol was distilled off under reduced pressure. A brown crude product obtained therefrom was dissolved in water (50 ml), and the solution was acidified with 1 M hydrochloric acid. Yellowish white precipitates generated therefrom were collected by filtration, washed with water, and then dissolved in tetrahydrofuran (50 ml). The solution was diluted with ethyl acetate (100 ml), and the dilution was washed with saturated brine, dried over anhydrous magnesium sulfate, and then concentrated under reduced pressure, to obtain an orange-colored solid. This solid was washed with diethyl ether, and thus the title compound (2.56 g) was obtained as a yellowish white solid.

¹H NMR (300 MHz, DMSO-d₆) δ ppm 4.82 (2H, q, J=8.9 Hz), 6.02 (1H, dd, J=2.5, 1.0 Hz), 7.10 (2H, d, J=9.0 Hz), 7.17 (2H, d, J=9.0 Hz), 7.34 (1H, t, J=2.5 Hz), 12.30 (1H, br. s.), 12.92 (1H, br. s.).

### Example 3

### 3-[4-(Cyclobutylmethoxy)phenyl]-2-thioxo-1,2,3,5-tetrahydro-4H-pyrrolo[3,2-d]pyrimidin-4-one

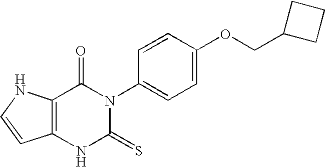

Ethyl 3-isothiocyanato-1H-pyrrol-2-carboxylate (500 mg) obtained by the method of Reference Example 31, or a method pursuant to thereto, and 4-(cyclobutylmethoxy)aniline (460 mg) obtained by the method of Reference Example 8, or a method pursuant to thereto, were added to acetonitrile (5 ml). The resulting mixture was stirred for 4 hours at 70° C., and then was concentrated under reduced pressure, to obtain a crude solid. This crude solid was added to a solution of potassium tert-butoxide (582 mg) in ethanol (5 ml), and the resulting mixture was stirred for 24 hours at room temperature. Subsequently, 1 M hydrochloric acid was added thereto until the pH value reached 6. A solid precipitated therefrom was collected by filtration, washed with water and petroleum ether, and then dried under reduced pressure, and thus the title compound (365 mg) was obtained.

MS(ESI+):328(M+H).

### Example 4

### 2-Thioxo-3-[4-(3,3,3-trifluoropropoxy)phenyl]-1,2,3,5-tetrahydro-4H-pyrrolo[3,2-d]pyrimidin-4-one

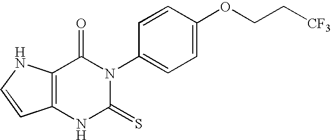

Ethyl 3-isothiocyanato-1H-pyrrol-2-carboxylate (500 mg) obtained by the method of Reference Example 31, or a method pursuant to thereto, and 4-(3,3,3-trifluoropropoxy)aniline (533 mg) obtained by the method of Reference Example 11, or a method pursuant to thereto, were added to acetonitrile (5 ml). The resulting mixture was stirred for 4 hours at 70° C., and then was concentrated under reduced pressure, to obtain a crude solid. This crude solid was added to a solution of potassium tert-butoxide (582 mg) in ethanol (5 ml), and the resulting mixture was stirred for 24 hours at room temperature. Subsequently, 1 M hydrochloric acid was added thereto until the pH value reached 6. A solid precipitated therefrom was collected by filtration, washed with water and petroleum ether, and then dried under reduced pressure, and thus the title compound (432 mg) was obtained.

MS(ESI+):356(M+H).

### Example 5

### 3-(4-Butoxyphenyl)-2-thioxo-1,2,3,5-tetrahydro-4H-pyrrolo[3,2-d]pyrimidin-4-one

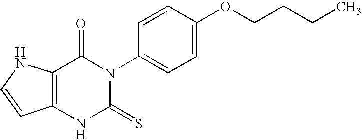

Ethyl 3-isothiocyanato-1H-pyrrol-2-carboxylate (500 mg) obtained by the method of Reference Example 31, or a method pursuant to thereto, and 4-butoxyaniline (429 mg) obtained by the method of Reference Example 12, or a method pursuant to thereto, were added to acetonitrile (5 ml). The resulting mixture was stirred for 4 hours at 70° C., and then was concentrated under reduced pressure, to obtain a crude solid. This crude solid was added to a solution of potassium tert-butoxide (582 mg) in ethanol (5 ml), and the resulting mixture was stirred for 24 hours at room temperature. Subsequently, 1 M hydrochloric acid was added thereto until the pH value reached 6. A solid precipitated therefrom was collected by filtration, washed with water and petroleum ether, and then dried under reduced pressure, and thus the title compound (567 mg) was obtained.

MS(ESI+):316(M+H).

### Example 6

### 3-[4-(Cyclopropylmethoxy)phenyl]-2-thioxo-1,2,3,5-tetrahydro-4H-pyrrolo[3,2-d]pyrimidin-4-one

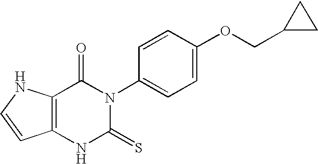

Ethyl 3-isothiocyanato-1H-pyrrol-2-carboxylate (500 mg) obtained by the method of Reference Example 31, or a method pursuant to thereto, and 4-(cyclopropylmethoxy)aniline (424 mg) obtained by the method of Reference Example 13, or a method pursuant to thereto, were added to acetonitrile (5 ml). The resulting mixture was stirred for 4 hours at 70° C., and then was concentrated under reduced pressure, to obtain a crude solid. This crude solid was added to a solution of potassium tert-butoxide (582 mg) in ethanol (5 ml), and the resulting mixture was stirred for 24 hours at room temperature. Subsequently, 1 M hydrochloric acid was added thereto until the pH value reached 6. A solid precipitated therefrom was collected by filtration, washed with water and petroleum ether, and then dried under reduced pressure, and thus the title compound (425 mg) was obtained.

MS(ESI+):314(M+H).

### Example 7

### 3-{4-[(1-Methylcyclopropyl)methoxy]phenyl}-2-thioxo-1,2,3,5-tetrahydro-4H-pyrrolo[3,2-d]pyrimidin-4-one

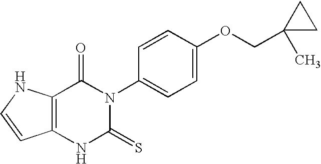

Ethyl 3-isothiocyanato-1H-pyrrol-2-carboxylate (500 mg) obtained by the method of Reference Example 31, or a method pursuant to thereto, and 4-[(1-methylcyclopropyl)methoxy]aniline (460 mg) obtained by the method of Reference Example 14, or a method pursuant to thereto, were introduced into acetonitrile (5 ml), and the resulting mixture was stirred for 4 hours at 70° C., and then was concentrated under reduced pressure, to obtain a crude solid. This crude solid was added to a solution of potassium tert-butoxide (582 mg) in ethanol (5 ml), and the resulting mixture was stirred for 24 hours at room temperature. Subsequently, 1 M hydrochloric acid was added thereto until the pH value reached 6. A solid precipitated therefrom was collected by filtration, washed with water and petroleum ether, and then dried under reduced pressure, and thus the title compound (517 mg) was obtained.

MS(ESI+):328(M+H).

### Example 8

### 3-[4-(3,3-Dimethylbutoxy)phenyl]-2-thioxo-1,2,3,5-tetrahydro-4H-pyrrolo[3,2-d]pyrimidin-4-one

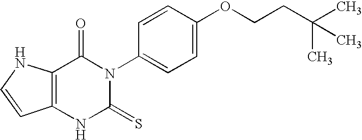

Ethyl 3-isothiocyanato-1H-pyrrol-2-carboxylate (500 mg) obtained by the method of Reference Example 31, or a method pursuant to thereto, and 4-(3,3-dimethylbutoxy)aniline (521 mg) obtained by the method of Reference Example 9, or a method pursuant to thereto, were added to acetonitrile (5 ml). The resulting mixture was stirred for 4 hours at 70° C., and then was concentrated under reduced pressure, to obtain a crude solid. This crude solid was added to a solution of potassium tert-butoxide (582 mg) in ethanol (5 ml), and the resulting mixture was stirred for 24 hours at room temperature. Subsequently, 1 M hydrochloric acid was added thereto until the pH value reached 6. A solid precipitated therefrom was collected by filtration, washed with water and petroleum ether, and then dried under reduced pressure, and thus the title compound (427 mg) was obtained.

MS(ESI+):344(M+H).

### Example 9

### 3-{4-[(2,2-Difluorocyclopropypmethoxy]phenyl}-2-thioxo-1,2,3,5-tetrahydro-4H-pyrro[3,2-d]pyrimidin-4-one

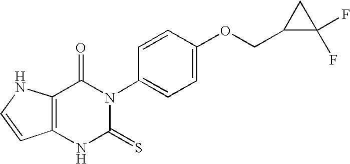

To 1-[(2,2-Difluorocyclopropyl)methoxy]-4-nitrobenzene (3.0 g) obtained by the method of Reference Example 10, or a method pursuant to thereto, 10% palladium/activated carbon (50% hydrated, 150 mg) and methanol (50 ml) were added, and the resulting mixture was stirred for 5 hours under a hydrogen atmosphere (40 psi). Subsequently, the reaction mixture was filtered, and the filtrate was concentrated under reduced pressure, to obtain 2.0 g of a black oily substance. This black oily substance (517 mg) and ethyl 3-isothiocyanato-1H-pyrrol-2-carboxylate (500 mg) obtained by the method of Reference Example 31, or a method pursuant to thereto, were added to acetonitrile (5 ml). The resulting mixture was stirred for 4 hours at 70° C., and then was concentrated under reduced pressure, to obtain a crude solid. This crude solid was added to a solution of potassium tert-butoxide (582 mg) in ethanol (5 ml), and the resulting mixture was stirred for 24 hours at room temperature. Subsequently, 1 M hydrochloric acid was added thereto until the pH value reached 6. A solid precipitated therefrom was collected by filtration, washed with water and petroleum ether, and then dried under reduced pressure, and thus the title compound (409 mg) was obtained.

MS(ESI+):350(M+H).

### Example 10

### 3-{4-[(2-Methylcyclopropyl)methoxy]phenyl}-2-thioxo-1,2,3,5-tetrahydro-4H-pyrrolo[3,2-d]pyrimidin-4-one

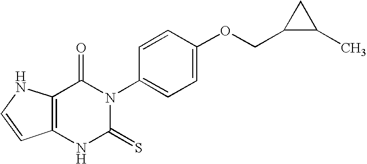

Ethyl 3-isothiocyanato-1H-pyrrol-2-carboxylate (500 mg) obtained by the method of Reference Example 31, or a method pursuant to thereto, and 4-[(2-methylcyclopropyl)methoxy]aniline (460 mg) obtained by the method of Reference Example 15, or a method pursuant to thereto, were added to acetonitrile (5 ml). The resulting mixture was stirred for 4 hours at 70° C., and then was concentrated under reduced pressure, to obtain a crude solid. This crude solid was added to a solution of potassium tert-butoxide (582 mg) in ethanol (5 ml), and the resulting mixture was stirred for 24 hours at room temperature. Subsequently, 1 M hydrochloric acid was added thereto until the pH value reached 6. A solid precipitated therefrom was collected by filtration, washed with water and petroleum ether, and then dried under reduced pressure, and thus the title compound (536 mg) was obtained.

MS (ESI+):328(M+H).

### Example 11

### 3-[4-(3-Methylbutoxy)phenyl]-2-thioxo-1,2,3,5-tetrahydro-4H-pyrrolo[3,2-d]pyrimidin-4-one

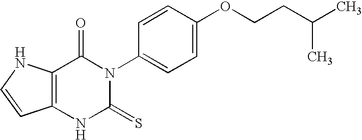

Ethyl 3-isothiocyanato-1H-pyrrol-2-carboxylate (500 mg) obtained by the method of Reference Example 31, or a method pursuant to thereto, and 4-(3-methylbutoxy)aniline (465 mg) obtained by the method of Reference Example 17, or a method pursuant to thereto, were added to acetonitrile (5 ml). The resulting mixture was stirred for 4 hours at 70° C., and then was concentrated under reduced pressure, to obtain a crude solid. This crude solid was added to a solution of potassium tert-butoxide (582 mg) in ethanol (5 ml), and the resulting mixture was stirred for 24 hours at room temperature. Subsequently, 1 M hydrochloric acid was added thereto until the pH value reached 6. A solid precipitated therefrom was collected by filtration, washed with water and petroleum ether, and then dried under reduced pressure, and thus the title compound (413 mg) was obtained.

MS(ESI+):330(M+H).

### Example 12

### 2-Thioxo-3-[4-(4,4,4-trifluorobutoxy)phenyl]-1,2,3,5-tetrahydro-4H-pyrrolo[3,2-d]pyrimidin-4-one

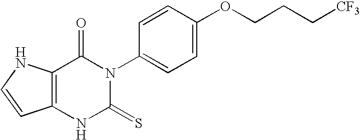

Ethyl 3-isothiocyanato-1H-pyrrol-2-carboxylate (500 mg) obtained by the method of Reference Example 31, or a method pursuant to thereto, and 4-(4,4,4-trifluorobutoxy)aniline (569 mg) obtained by the method of Reference Example 16, or a method pursuant to thereto, were added to acetonitrile (5 ml). The resulting mixture was stirred for 4 hours at 70° C., and then was concentrated under reduced pressure, to obtain a crude solid. This crude solid was added to a solution of potassium tert-butoxide (582 mg) in ethanol (5 ml), and the resulting mixture was stirred for 24 hours at room temperature. Subsequently, 1 M hydrochloric acid was added thereto until the pH value reached 6. A solid precipitated therefrom was collected by filtration, washed with water and petroleum ether, and then dried under reduced pressure, and thus the title compound (351 mg) was obtained.

MS(ESI+):370(M+H).

### Example 13

### 3-[4-(2,2-Dimethylpropoxy)phenyl]-2-thioxo-1,2,3,5-tetrahydro-4H-pyrrolo[3,2-d]pyrimidin-4-one

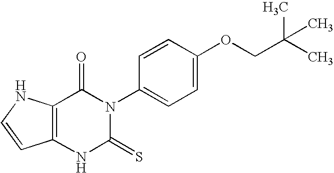

Ethyl 3-isothiocyanato-1H-pyrrol-2-carboxylate (500 mg) obtained by the method of Reference Example 31, or a method pursuant to thereto, and 4-(2,2-dimethylpropoxy)aniline (465 mg) obtained by the method of Reference Example 7, or a method pursuant to thereto, were added to acetonitrile (5 ml). The resulting mixture was stirred for 4 hours at 70° C., and then was concentrated under reduced pressure, to obtain a crude solid. This crude solid was added to a solution of potassium tert-butoxide (582 mg) in ethanol (5 ml), and the resulting mixture was stirred for 24 hours at room temperature. Subsequently, 1 M hydrochloric acid was added thereto until the pH value reached 6. A solid precipitated therefrom was collected by filtration, washed with water and petroleum ether, and then dried under reduced pressure, and thus the title compound (216 mg) was obtained.

MS(ESI+):330(M+H).

### Example 14

### 3-[4-(Cyclopropylmethoxy)-2-fluorophenyl]-2-thioxo-1,2,3,5-tetrahydro-4H-pyrrolo[3,2-d]pyrimidin-4-one

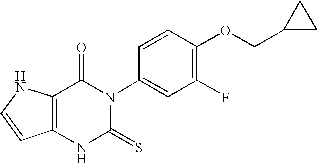

A mixture of ethyl 3-isothiocyanato-1H-pyrrol-2-carboxylate (1.0 g) obtained by the method of Reference Example 31, or a method pursuant to thereto, 4-(cyclopropylmethoxy)-2-fluoroaniline (924 mg) obtained by the method of Reference Example 18, or a method pursuant to thereto, and acetonitrile (20 ml) was heated to reflux for 3 hours. The mixture was ice-cooled, and then potassium tert-butoxide (2.36 g) and ethanol (20 ml) were added thereto. The resulting mixture was heated to reflux for one hour. The reaction mixture was returned to room temperature, and was acidified with 1 M hydrochloric acid. Precipitates generated therefrom were collected by filtration, washed with water and diethyl ether-hexane, and dried under reduced pressure. Thus, the title compound (1.26 g) was obtained as a pale yellow powder.

¹H NMR (300 MHz, DMSO-d₆) δ ppm 0.31-0.38 (2H, m), 0.57-0.64 (2H, m), 1.16-1.34 (1H, m), 3.88 (2H, d, J=7.0 Hz), 6.03 (1H, d, J=1.9 Hz), 6.81-6.85 (1H, m), 6.93 (1H, dd, J=12.1, 2.5 Hz), 7.22 (1H, dd, J=9.0, 8.8 Hz), 7.36-7.40 (1H, m), 12.37 (1H, br. s.), 13.05 (1H, s).

### Example 15

### 3-[4-(2-Cyclopropylethoxy)phenyl]-2-thioxo-1,2,3,5-tetrahydro-4H-pyrrolo[3,2-d]pyrimidin-4-one

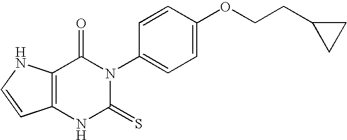

A mixture of ethyl 3-isothiocyanato-1H-pyrrol-2-carboxylate (1.00 g) obtained by the method of Reference Example 31, or a method pursuant to thereto, and 4-(2-cyclopropylethoxy)aniline (1.06 g) obtained in Reference Example 19 and acetonitrile (20 ml) was heated to reflux for 2 hours. A solution of potassium tert-butoxide (2.00 g) in ethanol (20 ml) was added to the mixture, and the resulting mixture was heated to reflux for one hour, and then was concentrated under reduced pressure. The residue was diluted with water, and then the pH was adjusted to about 4 with 1 M hydrochloric acid. A solid precipitated therefrom was collected by filtration, washed with water and diisopropyl ether, and then dried, and thus the title compound (1.63 g) was obtained as a pale brown solid.

¹H NMR (300 MHz, DMSO-d₆) δ ppm 0.12-0.18 (2H, m), 0.42-0.49 (2H, m), 0.80-0.95 (1H, m), 1.61-1.70 (2H, m), 4.06 (2H, t, J=6.6 Hz), 6.01 (1H, t, J=2.1 Hz), 6.98 (2H, d, J=8.7 Hz), 7.08 (2H, d, J=8.7 Hz), 7.34 (1H, t, J=2.8 Hz), 12.28 (1H, br. s.), 12.89 (1H, s).

### Example 16

### 2-Thioxo-3-{4-[(2,2,2-trifluoroethoxy)methyl]phenyl}-1,2,3,5-tetrahydro-4H-pyrrolo[3,2-d]pyrimidin-4-one

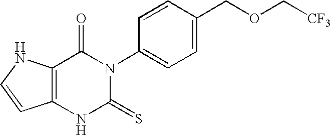

A mixture of ethyl 3-isothiocyanato-1H-pyrrol-2-carboxylate (0.50 g) obtained by the method of Reference Example 31, or a method pursuant to thereto, and 4-[(2,2,2-trifluoroethoxy)methyl]aniline (0.566 g) obtained in Reference Example 20 and acetonitrile (20 ml) was heated to reflux for 4 hours. A solution of potassium tert-butoxide (1.00 g) in ethanol (20 ml) was added to the mixture, and the resulting mixture was stirred for 2 hours at 100° C., and then was concentrated under reduced pressure. The residue was diluted with water, and then the pH was adjusted to about 5 with 1 M hydrochloric acid. A solid precipitated therefrom was collected by filtration, washed with water and diisopropyl ether, and then dried, to obtain a pale brown solid (0.524 g). This pale brown solid (50 mg) was recrystallized from ethyl acetate, and thus the title compound (20 mg) was obtained as a yellow solid.

¹H NMR (300 MHz, DMSO-d₆) δ ppm 4.18 (2H, q, J=9.4 Hz), 4.73 (2H, s), 6.03 (1H, d, J=2.7 Hz), 7.21 (2H, d, J=8.3 Hz), 7.35 (1H, d, J=1.9 Hz), 7.42 (2H, d, J=8.3 Hz), 12.31 (1H, br. s.), 12.94 (1H, br. s.).

### Example 17

### 3-[4-(Cyclopropylmethoxy)-3-methylphenyl]-2-thioxo-1,2,3,5-tetrahydro-4H-pyrrolo[3,2-d]pyrimidin-4-one

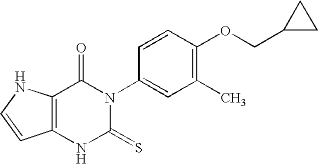

A mixture of ethyl 3-isothiocyanato-1H-pyrrol-2-carboxylate (1.00 g) obtained by the method of Reference Example 31, or a method pursuant to thereto, 4-(cyclopropylmethoxy)-3-methylaniline (0.975 g) obtained in Reference Example 21 and acetonitrile (20 ml) was heated to reflux for 4 hours. A solution of potassium tert-butoxide (2.00 g) in ethanol (20 ml) was added to the mixture, and the resulting mixture was heated to reflux for 2 hours, and then was concentrated under reduced pressure. The residue was diluted with water, and then the pH was adjusted to about 5 with 1 M hydrochloric acid. A solid precipitated therefrom was collected by filtration, washed with water and diisopropyl ether, and then dried, and thus the title compound (1.50 g) was obtained as a pale brown solid.

¹H NMR (300 MHz, DMSO-d₆) δ ppm 0.34-0.41 (2H, m), 0.56-0.64 (2H, m), 1.19-1.36 (1H, m), 2.17 (3H, s), 3.88 (2H, d, J=6.8 Hz), 6.01 (1H, t, J=2.3 Hz), 6.88-6.98 (3H, m), 7.33 (1H, t, J=3.0 Hz), 12.27 (1H, br. s.), 12.87 (1H, s).

### Example 18

### 3-[3-Chloro-4-(cyclopropylmethoxy)phenyl]-2-thioxo-1,2,3,5-tetrahydro-4H-pyrrolo[3,2-d]pyrimidin-4-one

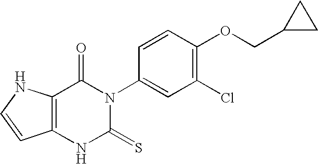

A mixture of ethyl 3-isothiocyanato-1H-pyrrol-2-carboxylate (1.00 g) obtained by the method of Reference Example 31, or a method pursuant to thereto, 3-chloro-4-(cyclopropylmethoxy)aniline (1.09 g) obtained in Reference Example 22 and acetonitrile (20 ml) was heated to reflux for 4 hours. A solution of potassium tert-butoxide (2.00 g) in ethanol (20 ml) was added to the mixture, and the resulting mixture was heated to reflux for 3 hours, and then was cooled to room temperature. A solid precipitated therefrom was collected by filtration, washed with acetonitrile, and then dissolved in water. The pH of the resulting aqueous solution was adjusted to about 6 with 1 M hydrochloric acid. A solid precipitated therefrom was collected by filtration, washed with water and then dried, and thus the title compound (0.928 g) was obtained as a pale brown solid.

¹H NMR (300 MHz, DMSO-d₆) δ ppm 0.34-0.42 (2H, m), 0.58-0.65 (2H, m), 1.22-1.37 (1H, m), 3.97 (2H, d, J=6.8 Hz), 6.02 (1H, d, J=2.7 Hz), 7.11 (1H, dd, J=9.1, 2.3 Hz), 7.16 (1H, d, J=9.1 Hz), 7.32-7.37 (2H, m), 12.31 (1H, br. s.), 12.94 (1H, br. s.).

### Example 19

### 6-Methyl-2-thioxo-3-[4-(2,2,2-trifluoroethoxy)phenyl]-1,2,3,5-tetrahydro-4H-pyrrolo[3,2-d]pyrimidin-4-one

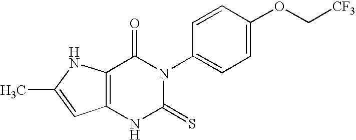

Ethyl 3-isothiocyanato-5-methyl-1H-pyrrol-2-carboxylate (410 mg) obtained by the method of Reference Example 32, or a method pursuant to thereto, and 4-(2,2,2-trifluoroethoxy)aniline (373 mg) were dissolved in acetonitrile (20 ml). The resulting mixture was heated to reflux for one hour. The reaction liquid was returned to room temperature, and then potassium tert-butoxide (901 mg) dissolved in ethanol (10 ml) was added to the reaction liquid. The mixture was heated to reflux again for one hour. The reaction mixture was returned to room temperature, and then was concentrated under reduced pressure, to obtain an orange-colored oily substance. This oily substance was dissolved in water (20 ml), and the solution was acidified with 1 M hydrochloric acid. Precipitates generated therefrom were collected by filtration, and were dissolved in tetrahydrofuran (50 ml). The solution was diluted with ethyl acetate (200 ml). The dilution was washed with saturated brine, dried over anhydrous magnesium sulfate, and then concentrated under reduced pressure. An orange-colored solid obtained therefrom was washed with a mixed solvent of 50% diethyl ether/hexane, and thus the title compound (457 mg) was obtained as a yellowish white solid.

¹H NMR (300 MHz, DMSO-d₆) δ ppm 2.29 (3H, s), 4.82 (2H, q, J=8.9 Hz), 5.81 (1H, d, J=1.5 Hz), 7.09 (2H, d, J=9.1 Hz), 7.14 (2H, d, J=9.1 Hz), 12.06 (1H, s), 12.81 (1H, s).

### Example 20

### 2-Thioxo-3-[4-(2,2,2-trifluoroethoxy)phenyl]-2,3,4a,5-tetrahydro-1H-pyrrolo[3,2-d]pyrimidine-4,6-dione

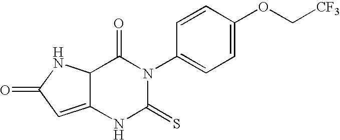

### Example 20a)

To a mixture of diethyl [(2,4-dimethoxybenzyl)amino]propanedioate (3.75 g) obtained by a method described in a published document, Tetrahedron, Vol. 39, p. 2399 (1983), or a method pursuant to thereto, cyanoacetic acid (0.98 g), 1-hydroxybenzotriazole (1.76 g) and N,N-dimethylformamide (50 ml), 1-ethyl-3-(3-dimethylaminopropyl)carbodiimide hydrochloride (2.65 g) was added, and the resulting mixture was stirred for 15 hours at room temperature. The solvent was distilled off at 50° C. under reduced pressure, and then the residue was diluted with ethyl acetate. The dilution was washed with water and saturated brine, dried over anhydrous magnesium sulfate, and then concentrated under reduced pressure. The resulting residue was purified by chromatography, and thus diethyl 3-amino-1-(2,4-dimethoxybenzyl)-5-oxo-1,5-dihydro-2H-pyrrol-2,2-dicarboxylate (3.30 g) was obtained as a white powder.

¹H NMR (300 MHz, DMSO-d₆) δ ppm 1.04 (6H, t, J=7.0 Hz), 3.71 (3H, s), 3.76 (3H, s), 3.83-4.08 (4H, m), 4.37 (2H, s), 4.69 (1H, s), 6.40 (1H, dd, J=8.3, 2.3 Hz), 6.50 (1H, d, J=2.3 Hz), 6.54 (2H, br. s.), 6.66 (1H, d, J=8.3 Hz).

### Example 20b)

Diethyl 3-amino-1-(2,4-dimethoxybenzyl)-5-oxo-1,5-dihydro-2H-pyrrol-2,2-dicarboxylate (785 mg) obtained by the method of Example (20a), or a method pursuant to thereto, was dissolved in N,N-dimethylformamide (5 ml), and this solution was added dropwise under ice cooling to a mixture of 1-isothiocyanato-4-(2,2,2-trifluoroethoxy)benzene (466 mg) obtained by the method of Reference Example 37, or a method pursuant to thereto, sodium hydride (60% in oil, 80 mg) and N,N-dimethylformamide (10 ml). The reaction mixture was stirred for 10 minutes at 0° C., and then was poured into 0.2 M hydrochloric acid (10 ml). The mixture was extracted with ethyl acetate, washed with water and saturated brine, dried over anhydrous magnesium sulfate, and then concentrated under reduced pressure, to obtain a yellow solid. This solid was purified by chromatography, and thus diethyl 1-(2,4-dimethoxybenzyl)-5-oxo-3-({[4-(2,2,2-trifluoroethoxy)phenyl]carbamothioyl}amino)-1,5-dihydro-2H-pyrrol-2,2-dicarboxylate (857 mg) was obtained as a yellowish white solid.

¹H NMR (300 MHz, DMSO-d₆) δ ppm 1.05 (6H, t, J=7.2 Hz), 3.72 (3H, s), 3.79 (3H, s), 3.88-4.21 (4H, m), 4.51 (2H, s), 4.77 (2H, q, J=8.9 Hz), 6.43 (1H, dd, J=8.3, 2.3 Hz), 6.54 (1H, d, J=2.3 Hz), 6.68 (1H, d, J=8.3 Hz), 7.08 (2H, d, J=9.1 Hz), 7.14 (1H, s), 7.46 (2H, d, J=9.1 Hz), 9.38 (1H, br. s.), 10.71 (1H, s).

### Example 20c)

Diethyl 1-(2,4-dimethoxybenzyl)-5-oxo-3-({[4-(2,2,2-trifluoroethoxy)phenyl]carbamothioyl}amino)-1,5-dihydro-2H-pyrrol-2,2-dicarboxylate (670 mg) obtained in Example (20b) was dissolved in 5% anisole/trifluoroacetic acid solution (12 ml), and the resulting solution was stirred for 3 days. Subsequently, the reaction solution was concentrated under reduced pressure, and was azeotropically boiled with toluene. The resulting residue was dissolved in ethyl acetate (50 ml), washed with a saturated aqueous solution of sodium hydrogen carbonate and saturated brine, dried over anhydrous magnesium sulfate, and then concentrated under reduced pressure. The resulting residue was purified by chromatography, and thus diethyl 5-oxo-3-({[4-(2,2,2-trifluoroethoxy)phenyl]carbamothioyl}amino)-1,5-dihydro-2H-pyrrol-2,2-dicarboxylate (436 mg) was obtained as a white solid.

¹H NMR (300 MHz, DMSO-d₆) δ ppm 1.22 (6H, t, J=7.2 Hz), 4.27 (4H, q, J=7.2 Hz), 4.77 (2H, q, J=8.9 Hz), 6.93 (1H, d, J=1.5 Hz), 7.08 (2H, d, J=9.0 Hz), 7.44 (2H, d, J=9.0 Hz), 9.01 (1H, d, J=1.5 Hz), 9.26 (1H, br. s.), 10.80 (1H, s).

### Example 20d)

Diethyl 5-oxo-3-({[4-(2,2,2-trifluoroethoxy)phenyl]carbamothioyl}amino)-1,5-dihydro-2H-pyrrol-2,2-dicarboxylate (429 mg) obtained in Example (20c) was dissolved in acetonitrile (10 ml), and a 1 M aqueous solution of sodium hydroxide (2.7 ml) was added to the solution under ice cooling. The mixture was stirred for 30 minutes at 0° C. Subsequently, the reaction mixture was poured into 0.1 M hydrochloric acid (50 ml), and then a precipitated brown solid was collected by filtration, washed with water, and then dissolved in tetrahydrofuran (10 ml). The solution was diluted with ethyl acetate (30 ml), and the dilution was washed with saturated brine, dried over anhydrous magnesium sulfate, and then concentrated under reduced pressure. A crude product obtained therefrom was washed with diethyl ether. Thus, the title compound, 2-thioxo-3-[4-(2,2,2-trifluoroethoxy)phenyl]-2,3,4a,5-tetrahydro-1H-pyrrolo[3,2-d]pyrimidine-4,6-dione (73 mg), was obtained as a yellowish white solid.

¹H NMR (300 MHz, DMSO-d₆) δ ppm 4.81 (2H, q, J=8.9 Hz), 5.10 (1H, d, J=2.3 Hz), 7.01-7.18 (4H, m), 11.55 (1H, br. s.), 11.66 (1H, d, J=2.3 Hz), 12.61 (1H, s).

### Example 21

### 7-Cyclopropyl-2-thioxo-3-[4-(2,2,2-trifluoroethoxy)phenyl]-1,2,3,5-tetrahydro-4H-pyrrolo[3,2-d]pyrimidin-4-one

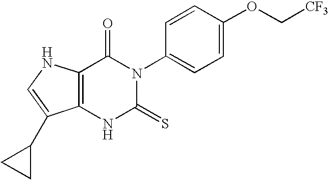

### Example 21a)

Ethyl 4-cyclopropyl-3-isothiocyanato-1H-pyrrol-2-carboxylate (1.18 g) obtained by the method of Reference Example 35, or a method pursuant to thereto, and 4-(2,2,2-trifluoroethoxy)aniline (955 mg) were dissolved in acetonitrile (25 ml), and the mixture was heated to reflux for 2 hours. The reaction mixture was returned to room temperature, and then was concentrated under reduced pressure. A light yellow crude product obtained therefrom was purified by chromatography, and thus ethyl 4-cyclopropyl-3-({[4-(2,2,2-trifluoroethoxy)phenyl]carbamothioyl}amino)-1H-pyrrol-2-carboxylate (1.03 g) was obtained as a light yellow oily substance.

¹H NMR (300 MHz, DMSO-d₆) δ ppm 0.44-0.57 (2H, m), 0.65-0.79 (2H, m), 1.25 (3H, t, J=7.1 Hz), 1.48-1.73 (1H, m), 4.18 (2H, q, J=7.1 Hz), 4.73 (2H, q, J=8.9 Hz), 6.64 (1H, d, J=3.4 Hz), 6.99 (2H, d, J=9.1 Hz), 7.37 (2H, d, J=9.1 Hz), 8.93 (1H, s), 9.14 (1H, br. s.), 11.54 (1H, d, J=3.4 Hz).

### Example 21b)

Ethyl 4-cyclopropyl-3-({[4-(2,2,2-trifluoroethoxy)phenyl]carbamothioyl}amino)-1H-pyrrol-2-carboxylate (1.03 g) obtained in Example (21a) was dissolved in ethanol (25 ml), and a 20% sodium ethoxide-ethanol solution (2.54 g) was added thereto. The mixture was heated to reflux for one hour. The reaction solution was returned to room temperature, and then was concentrated under reduced pressure. A yellow solid obtained therefrom was dissolved in water (20 ml), and the solution was acidified with 1 M hydrochloric acid under ice cooling. White precipitates generated therefrom were collected by filtration, washed with water, and then dissolved in tetrahydrofuran (30 ml). The solution was diluted with ethyl acetate (150 ml), and the dilution was washed with saturated brine, dried over anhydrous magnesium sulfate, and then concentrated under reduced pressure, to obtain a light yellow solid. This solid was washed with diethyl ether. Thus, the title compound, 7-cyclopropyl-2-thioxo-3-[4-(2,2,2-trifluoroethoxy)phenyl]-1,2,3,5-tetrahydro-4H-pyrrolo[3,2-d]pyrimidin-4-one (775 mg), was obtained as a white powder.

¹H NMR (300 MHz, DMSO-d₆) ppm 0.51-0.59 (2H, m), 0.77-0.86 (2H, m), 1.98-2.11 (1H, m), 4.82 (2H, q, J=9.0 Hz), 7.02 (1H, d, J=2.6 Hz), 7.10 (2H, d, J=9.2 Hz), 7.15 (2H, d, J=9.2 Hz), 12.00 (1H, d, J=2.6 Hz), 13.01 (1H, s).

### Example 22

### 7-Ethyl-2-thioxo-3-[4-(2,2,2-trifluoroethoxy)phenyl]-1,2,3,5-tetrahydro-4H-pyrrolo[3,2-d]pyrimidin-4-one

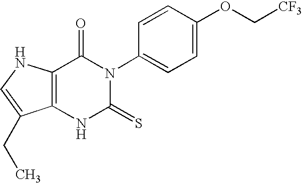

Ethyl 4-ethyl-3-isothiocyanato-1H-pyrrol-2-carboxylate (1.24 g) obtained by the method of Reference Example 34, or a method pursuant to thereto, and 4-(2,2,2-trifluoroethoxy)aniline (1.06 g) were dissolved in acetonitrile (25 ml), and the mixture was heated to reflux for 2 hours. The reaction solution was returned to room temperature, and then was concentrated under reduced pressure, to obtain a light yellow crude product. Ethanol (25 ml) was added to this crude product, and then a 20% sodium ethoxide-ethanol solution (5.64 g) was added thereto. The reaction solution was returned to room temperature, and then was concentrated under reduced pressure. Water (40 ml) was added to the crude product, and the mixture was acidified with 1 M hydrochloric acid under ice cooling. Yellowish white precipitates generated therefrom were collected by filtration, and were dissolved in tetrahydrofuran (100 ml). The solution was diluted with ethyl acetate (300 ml), and the dilution was washed with saturated brine, dried over anhydrous magnesium sulfate, and then concentrated under reduced pressure, to obtain a light yellow solid. This solid was washed with diethyl ether and then purified by chromatography, and thus the title compound (885 mg) was obtained as a white solid.

¹H NMR (300 MHz, DMSO-d₆) δ ppm 1.14 (3H, t, J=7.6 Hz), 2.60 (2H, q, J=7.6 Hz), 4.82 (2H, q, J=9.0 Hz), 7.10 (2H, d, J=9.1 Hz), 7.15 (2H, d, J=9.1 Hz), 7.18 (1H, d, J=2.8 Hz), 12.04 (1H, br. s.), 12.89 (1H, s).

### Example 23

### 7-Methyl-2-thioxo-3-[4-(2,2,2-trifluoroethoxy)phenyl]-1,2,3,5-tetrahydro-4H-pyrrolo[3,2-d]pyrimidin-4-one

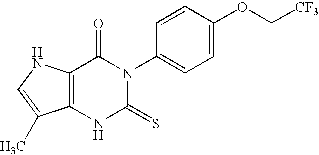

Ethyl 3-isothiocyanato-4-methyl-1H-pyrrol-2-carboxylate (265 mg) obtained by the method of Reference Example 33, or a method pursuant to thereto, and 4-(2,2,2-trifluoroethoxy)aniline (241 mg) were dissolved in acetonitrile (13 ml). The resulting mixture was heated to reflux for one hour. The reaction mixture was returned to room temperature, and then potassium tert-butoxide (582 mg) dissolved in ethanol (5 ml) was added to the reaction liquid. The mixture was heated to reflux again for one hour. The reaction mixture was returned to room temperature, and then was concentrated under reduced pressure, to obtain an orange-colored oily substance. This oily substance was dissolved in water (20 ml), and the solution was acidified with 1 M hydrochloric acid. Precipitates generated therefrom were collected by filtration, and were dissolved in tetrahydrofuran (50 ml). The solution was diluted with ethyl acetate (200 ml). The dilution was washed with saturated brine, dried over anhydrous magnesium sulfate, and then concentrated under reduced pressure. An orange-colored solid obtained therefrom was washed with diethyl ether, to obtain a crude mixture (170 mg).

MS(ESI+):356(M+H).

### Example 24

### 3-(4-Ethoxyphenyl)-2-thioxo-1,2,3,7-tetrahydro-4H-pyrrolo[2,3-d]pyrimidin-4-one

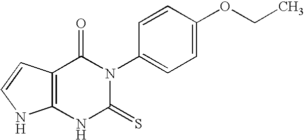

### Example 24a)

Ethyl 2-isothiocyanato-1H-pyrrol-3-carboxylate (717 mg) obtained by the method of Reference Example 36, or a method pursuant to thereto, and 4-ethoxyaniline (552 mg) were dissolved in acetonitrile (18 ml), and the solution was heated to reflux for one hour under a nitrogen atmosphere. The reaction solution was returned to room temperature, and then was concentrated under reduced pressure. A crude product obtained therefrom was purified by chromatography, to obtain a yellowish white solid. This solid was washed with a mixed solvent of diethyl ether/hexane, and thus ethyl 2-{[(4-ethoxyphenyl)carbamothioyl]amino}-1H-pyrrol-3-carboxylate (1.13 g) was obtained as a white solid.

¹H NMR (400 MHz, DMSO-d₆) δ ppm 1.20 (3H, t, J=7.0 Hz), 1.34 (3H, t, J=7.0 Hz), 4.04 (2H, q, J=7.0 Hz), 4.12 (2H, q, J=7.0 Hz), 6.25 (1H, t, J=2.9 Hz), 6.49 (1H, dd, J=2.9, 2.7 Hz), 6.96 (2H, d, J=8.8 Hz), 7.31 (2H, d, J=8.8 Hz), 10.11 (1H, s), 10.57 (1H, br. s.), 12.06 (1H, br. s.).

### Example 24b)

Ethyl 2-{[(4-ethoxyphenyl)carbamothioyl]amino}-1H-pyrrol-3-carboxylate (667 mg) obtained by the method of Example (24a), or a method pursuant to thereto, was dissolved in ethanol (10 ml), and a 20% sodium ethoxide-ethanol solution (3.4 g) was added thereto. The mixture was heated to reflux for 30 minutes. The reaction solution was returned to room temperature, and then was concentrated under reduced pressure. Water (20 ml) was added to a crude product obtained therefrom, and the mixture was acidified (near pH 5) with 1 M hydrochloric acid. Precipitates generated therefrom were collected by filtration, and were dissolved in tetrahydrofuran (50 ml). The solution was diluted with ethyl acetate (100 ml), and the dilution was washed with saturated brine, dried over anhydrous magnesium sulfate, and then concentrated under reduced pressure. An orange-colored solid obtained therefrom was washed with a mixed solvent of 10% tetrahydrofuran/diethyl ether, and thus the title compound (544 mg) was obtained as a brown solid.

¹H NMR (400 MHz, DMSO-d₆) δ ppm 1.36 (3H, t, J=7.0 Hz), 4.06 (2H, q, J=7.0 Hz), 6.38 (1H, d, J=3.4 Hz), 6.77 (1H, d, J=3.4 Hz), 6.95 (2H, d, J=8.8 Hz), 7.04 (2H, d, J=8.8 Hz), 11.27 (1H, br. s.), 13.51 (1H, br. s.).

### Example 25

### 3-[4-(2,2-Dimethylpropoxy)phenyl]-2-thioxo-1,2,3,7-tetrahydro-4H-pyrrolo[2,3-d]pyrimidin-4-one

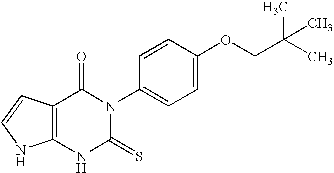

Ethyl 2-isothiocyanato-1H-pyrrol-3-carboxylate (400 mg) obtained by the method of Reference Example 36, or a method pursuant to thereto, and 4-(2,2-dimethylpropoxy)aniline (350 mg) obtained by the method of Reference Example 7, or a method pursuant to thereto, were added to acetonitrile (5 ml). The resulting mixture was stirred for 4 hours at 70° C., and then was concentrated under reduced pressure, to obtain a crude solid. This crude solid was added to a solution of potassium tert-butoxide (440 mg) in ethanol (5 ml), and the resulting mixture was stirred for 24 hours at room temperature. Subsequently, 1 M hydrochloric acid was added thereto until the pH value reached 6. A solid precipitated therefrom was collected by filtration, washed with water and petroleum ether, and then dried under reduced pressure, and thus the title compound (235 mg) was obtained.

MS(ESI+):330(M+H).

### Example 26

### 2-Thioxo-3-[4-(2,2,2-trifluoroethoxy)phenyl]-1,2,3,7-tetrahydro-4H-pyrrolo[2,3-d]pyrimidin-4-one

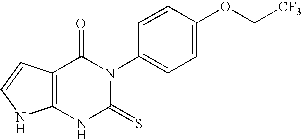

Ethyl 2-isothiocyanato-1H-pyrrol-3-carboxylate (1.45 g) obtained by the method of Reference Example 36, or a method pursuant to thereto, and 4-(2,2,2-trifluoroethoxy)aniline (1.41 g) were dissolved in acetonitrile (74 ml), and the solution was heated to reflux for one hour under a nitrogen atmosphere. After consumption of ethyl 2-isothiocyanato-1H-pyrrol-3-carboxylate was confirmed by TLC, the reaction solution was concentrated under reduced pressure to obtain a pale yellow solid. This solid was suspended in ethanol (50 ml) and a 20% sodium ethoxide-ethanol solution (7.79 g) was added to the suspension, then the suspension was turned into a solution. The solution was heated to reflux for additional one hour under a nitrogen atmosphere. The reaction solution was cooled to room temperature and was concentrated under reduced pressure. Water (60 ml) was added to the residue and the mixture was acidified with 1 M hydrochloric acid under ice cooling. Beige precipitates generated therefrom were collected by filtration and were dissolved in tetrahydrofuran (60 ml). The solution was diluted with ethyl acetate (240 ml), washed with saturated brine, dried over anhydrous magnesium sulfate and concentrated under reduced pressure to give a beige solid, which was washed with diethyl ether to give the title compound (2.36 g) as a beige solid.

¹H NMR (300 MHz, DMSO-d₆) δ ppm 4.82 (2H, q, J=8.9 Hz), 6.39 (1H, d, J=3.4 Hz), 6.78 (1H, d, J=3.4 Hz), 7.09 (2H, d, J=9.2 Hz), 7.13 (2H, d, J=9.2 Hz), 11.27 (1H, br. s.), 13.52 (1H, br. s.).

### Example 27

### 3-[4-(3,3-Dimethylbutoxy)pheny]-2-thioxo-1,2,3,7-tetrahydro-4H-pyrrolo[2,3-d]pyrimidin-4-one

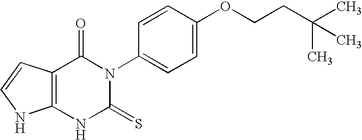

Ethyl 2-isothiocyanato-1H-pyrrol-3-carboxylate (400 mg) obtained by the method of Reference Example 36, or a method pursuant to thereto, and 4-(3,3-dimethylbutoxy)aniline (386 mg) obtained by the method of Reference Example 9, or a method pursuant to thereto, were added to acetonitrile (5 ml). The resulting mixture was stirred for 4 hours at 70° C., and then was concentrated under reduced, pressure, to obtain a crude solid. This crude solid was added to a solution of potassium tert-butoxide (440 mg) in ethanol (5 ml), and the resulting mixture was stirred for 24 hours at room temperature. Subsequently, 1 M hydrochloric acid was added thereto until the pH value reached 6. A solid precipitated therefrom was collected by filtration, washed with water and petroleum ether, and then dried under reduced pressure, and thus the title compound (536 mg) was obtained.

MS(ESI+):344(M+H).

### Example 28

### 3-{4-[(2,2-Difluorocyclopropypmethoxy]phenyl}-2-thioxo-1,2,3,7-tetrahydro-4H-pyrrolo[2,3-d]pyrimidin-4-one

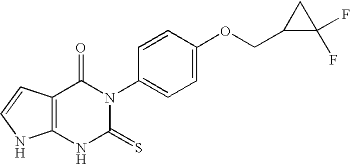

To 1-[(2,2-difluorocyclopropyl)methoxy]-4-nitrobenzene (3.0 g) obtained by the method of Reference Example 10, or a method pursuant to thereto, 10% palladium/activated carbon (50% hydrated, 150 mg) and methanol (50 ml) were added, and the resulting mixture was stirred for 5 hours under a hydrogen atmosphere (40 psi). Subsequently, the reaction mixture was filtered, and the filtrate was concentrated under reduced pressure, to obtain 2.0 g of a black oily substance. This black oily substance (517 mg) and ethyl 2-isothiocyanato-1H-pyrrol-3-carboxylate (400 mg) obtained by the method of Reference Example 36, or a method pursuant to thereto, were added to acetonitrile (5 ml). The resulting mixture was stirred for 4 hours at 70° C. Then, the mixture was concentrated under reduced pressure, to obtain a crude solid. This crude solid was added to a solution of potassium tert-butoxide (440 mg) in ethanol (5 ml), and the resulting mixture was stirred for 24 hours at room temperature. Subsequently, 1 M hydrochloric acid was added thereto until the pH value reached 6. A solid precipitated therefrom was collected by filtration, washed with water and petroleum ether, and then dried under reduced pressure, and thus the title compound (466 mg) was obtained.

MS(ESI+):350(M+H).

### Example 29

### 3-[4-(Cyclobutylmethoxy)phenyl]-2-thioxo-1,2,3,7-tetrahydro-4H-pyrrolo[2,3-d]pyrimidin-4-one

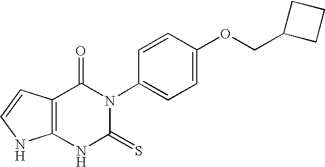

Ethyl 2-isothiocyanato-1H-pyrrol-3-carboxylate (400 mg) obtained by the method of Reference Example 36, or a method pursuant to thereto, and 4-(cyclobutylmethoxy)aniline (354 mg) obtained by the method of Reference Example 8, or a method pursuant to thereto, were added to acetonitrile (5 ml). The resulting mixture was stirred for 4 hours at 70° C., and then was concentrated under reduced pressure, to obtain a crude solid. This crude solid was added to a solution of potassium tert-butoxide (440 mg) in ethanol (5 ml), and the resulting mixture was stirred for 24 hours at room temperature. Subsequently, 1 M hydrochloric acid was added thereto until the pH value reached 6. A solid precipitated therefrom was collected by filtration, washed with water and petroleum ether, and then dried under reduced pressure, and thus the title compound (346 mg) was obtained.

MS(ESI+):328(M+H).

### Example 30

### 3-(4-Butoxyphenyl)-2-thioxo-1,2,3,7-tetrahydro-4H-pyrrolo[2,3-d]pyrimidin-4-one

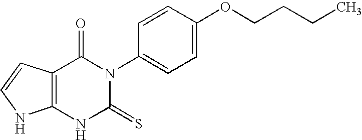

Ethyl 2-isothiocyanato-1H-pyrrol-3-carboxylate (400 mg) obtained by the method of Reference Example 36, or a method pursuant to thereto, and 4-butoxyaniline (330 mg) obtained by the method of Reference Example 12, or a method pursuant to thereto, were added to acetonitrile (5 ml). The resulting mixture was stirred for 4 hours at 70° C., and then was concentrated under reduced pressure, to obtain a crude solid. This crude solid was added to a solution of potassium tert-butoxide (440 mg) in ethanol (5 ml), and the resulting mixture was stirred for 24 hours at room temperature. Subsequently, 1 M hydrochloric acid was added thereto until the pH value reached 6. A solid precipitated therefrom was collected by filtration, washed with water and petroleum ether, and then dried under reduced pressure, and thus the title compound (254 mg) was obtained.

MS(ESI+):316(M+H).

### Example 31

### 3-[4-(Cyclopropylmethoxy)phenyl]-2-thioxo-1,2,3,7-tetrahydro-4H-pyrrolo[2,3-d]pyrimidin-4-one

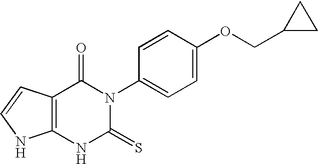

Ethyl 2-isothiocyanato-1H-pyrrol-3-carboxylate (400 mg) obtained by the method of Reference Example 36, or a method pursuant to thereto, and 4-(cyclopropylmethoxy)aniline (326 mg) obtained by the method of Reference Example 13, or a method pursuant to thereto, were added to acetonitrile (5 ml). The resulting mixture was stirred for 4 hours at 70° C., and then was concentrated under reduced pressure, to obtain a crude solid. This crude solid was added to a solution of potassium tert-butoxide (440 mg) in ethanol (5 ml), and the resulting mixture was stirred for 24 hours at room temperature. Subsequently, 1 M hydrochloric acid was added thereto until the pH value reached 6. A solid precipitated therefrom was collected by filtration, washed with water and petroleum ether, and then dried under reduced pressure, and thus the title compound (403 mg) was obtained.

MS(ESI+):314(M+H).

### Example 32

### 2-Thioxo-3-[4-(4,4,4-trifluorobutoxy)phenyl]-1,2,3,7-tetrahydro-4H-pyrrolo[2,3-d]pyrimidin-4-one

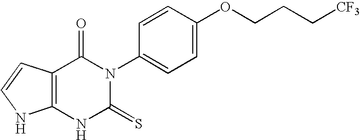

Ethyl 2-isothiocyanato-1H-pyrrol-3-carboxylate (400 mg) obtained by the method of Reference Example 36, or a method pursuant to thereto, and 4-(4,4,4-trifluorobutoxy)aniline (430 mg) obtained by the method of Reference Example 16, or a method pursuant to thereto, were added to acetonitrile (5 ml). The resulting mixture was stirred for 4 hours at 70° C., and then was concentrated under reduced pressure, to obtain a crude solid. This crude solid was added to a solution of potassium tert-butoxide (440 mg) in ethanol (5 ml), and the resulting mixture was stirred for 24 hours at room temperature. Subsequently, 1 M hydrochloric acid was added thereto until the pH value reached 6. A solid precipitated therefrom was collected by filtration, washed with water and petroleum ether, and then dried under reduced pressure, and thus the title compound (213 mg) was obtained.

MS(ESI+):370(M+H).

### Example 33

### 3-{4-[(2-Methylcyclopropyl)methoxy]phenyl}-2-thioxo-1,2,3,7-tetrahydro-4H-pyrrolo[2,3-d]pyrimidin-4-one

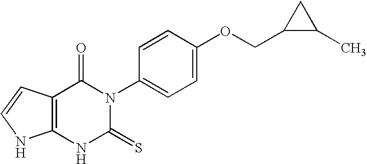

Ethyl 2-isothiocyanato-1H-pyrrol-3-carboxylate (400 mg) obtained by the method of Reference Example 36, or a method pursuant to thereto, and 4-[(2-methylcyclopropyl)methoxy]aniline (354 mg) obtained by the method of Reference Example 15, or a method pursuant to thereto, were added to acetonitrile (5 ml). The resulting mixture was stirred for 4 hours at 70° C., and then was concentrated under reduced pressure, to obtain a crude solid. This crude solid was added to a solution of potassium tert-butoxide (440 mg) in ethanol (5 ml), and the resulting mixture was stirred for 24 hours at room temperature. Subsequently, 1 M hydrochloric acid was added thereto until the pH value reached 6. A solid precipitated therefrom was collected by filtration, washed with water and petroleum ether, and then dried under reduced pressure, and thus the title compound (268 mg) was obtained.

MS(ESI+):328(M+H).

### Example 34

### 3-{4-[(1-Methylcyclopropyl)methoxy]phenyl}-2-thioxo-1,2,3,7-tetrahydro-4H-pyrrolo[2,3-d]pyrimidin-4-one

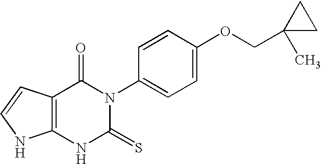

Ethyl 2-isothiocyanato-1H-pyrrol-3-carboxylate (400 mg) obtained by the method of Reference Example 36, or a method pursuant to thereto, and 4-[(1-methylcyclopropyl)methoxy]aniline (354 mg) obtained by the method of Reference Example 14, or a method pursuant to thereto, were added to acetonitrile (5 ml). The resulting mixture was stirred for 4 hours at 70° C., and then was concentrated under reduced pressure, to obtain a crude solid. This crude solid was added to a solution of potassium tert-butoxide (440 mg) in ethanol (5 ml), and the resulting mixture was stirred for 24 hours at room temperature. Subsequently, 1 M hydrochloric acid was added thereto until the pH value reached 6. A solid precipitated therefrom was collected by filtration, washed with water and petroleum ether, and then dried under reduced pressure, and thus the title compound (309 mg) was obtained.

MS(ESI+):328(M+H).

### Example 35

### 2-Thioxo-3-[4-(3,3,3-trifluoropropoxy)phenyl]-1,2,3,7-tetrahydro-4H-pyrrolo[2,3-d]pyrimidin-4-one

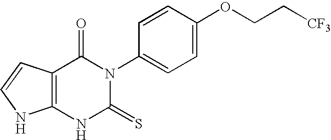

Ethyl 2-isothiocyanato-1H-pyrrol-3-carboxylate (400 mg) obtained by the method of Reference Example 36, or a method pursuant to thereto, and 4-(3,3,3-trifluoropropoxy)aniline (418 mg) obtained by the method of Reference Example 11, or a method pursuant to thereto, were added to acetonitrile (5 ml). The resulting mixture was stirred for 4 hours at 70° C., and then was concentrated under reduced pressure, to obtain a crude solid. This crude solid was added to a solution of potassium tert-butoxide (440 mg) in ethanol (5 ml), and the resulting mixture was stirred for 24 hours at room temperature. Subsequently, 1 M hydrochloric acid was added thereto until the pH value reached 6. A solid precipitated therefrom was collected by filtration, washed with water and petroleum ether, and then dried under reduced pressure, and thus the title compound (324 mg) was obtained.

MS(ESI+):356(M+H).

### Example 36

### 3-[4-(3-Methylbutoxy)phenyl]-2-thioxo-1,2,3,7-tetrahydro-4H-pyrrolo[2,3-d]pyrimidin-4-one

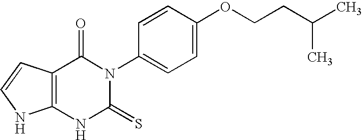

Ethyl 2-isothiocyanato-1H-pyrrol-3-carboxylate (400 mg) obtained by the method of Reference Example 36, or a method pursuant to thereto, and 4-(3-methylbutoxy)aniline (350 mg) obtained by the method of Reference Example 17, or a method pursuant to thereto, were added to acetonitrile (5 ml). The resulting mixture was stirred for 4 hours at 70° C., and then was concentrated under reduced pressure, to obtain a crude solid. This crude solid was added to a solution of potassium tert-butoxide (440 mg) in ethanol (5 ml), and the resulting mixture was stirred for 24 hours at room temperature. Subsequently, 1 M hydrochloric acid was added thereto until the pH value reached 6. A solid precipitated therefrom was collected by filtration, washed with water and petroleum ether, and then dried under reduced pressure, and thus the title compound (204 mg) was obtained.

MS(ESI+):330(M+H).

### Example 37

### 2-Thioxo-3-[4-(trifluoromethoxy)phenyl]-1,2,3,7-tetrahydro-4H-pyrrolo[2,3-d]pyrimidin-4-one

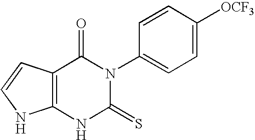

A mixture of ethyl 2-isothiocyanato-1H-pyrrol-3-carboxylate (500 mg) obtained by the method of Reference Example 36, or a method pursuant to thereto, 4-(trifluoromethoxy)aniline (451 mg) and acetonitrile (10 ml) was heated to reflux for one hour. The mixture was ice-cooled, and then a 20% sodium ethoxide-ethanol solution (3.5 ml) and ethanol (3.5 ml) were added thereto. The mixture was stirred for 2 hours at 95° C. The reaction mixture was returned to room temperature, and was acidified with 1 M hydrochloric acid. Precipitates generated therefrom were collected by filtration. These precipitates were washed with water and diethyl ether, and were dried under reduced pressure, and thus the title compound (689 mg) was obtained as a pale brown powder.

¹H NMR (300 MHz, DMSO-d₆) δ ppm 6.41 (1H, dd, J=3.3, 2.2 Hz), 6.79 (1H, dd, J=3.3, 2.2 Hz), 7.29-7.38 (2H, m), 7.40-7.49 (2H, m), 11.33 (1H, br. s.), 13.62 (1H, s).

### Example 38

### 3-[3-Chloro-4-(2,2,2-trifluoroethoxy)phenyl]-2-thioxo-1,2,3,7-tetrahydro-4H-pyrrolo[2,3-d]pyrimidin-4-one

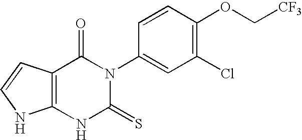

A mixture of ethyl 2-isothiocyanato-1H-pyrrol-3-carboxylate (500 mg) obtained by the method of Reference Example 36, or a method pursuant to thereto, 3-chloro-4-(2,2,2-trifluoroethoxy)aniline (575 mg) obtained by the method of Reference Example 23, or a method pursuant to thereto, and acetonitrile (20 ml) was heated to reflux for 2 hours. The mixture was ice-cooled, and then a 20% sodium ethoxide-ethanol solution (3.5 ml) and ethanol (3.5 ml) were added thereto. The mixture was stirred for 2 hours at 95° C. The reaction mixture was returned to room temperature, and the solvent was distilled off under reduced pressure. The residue was made basic with a 1 M aqueous solution of sodium hydroxide, and then was washed with a mixed solvent of 25% tetrahydrofuran/diethyl ether. The aqueous layer obtained therefrom was acidified with 1 M hydrochloric acid, and then was extracted with ethyl acetate. The organic layer obtained therefrom was washed with saturated brine, dried over anhydrous magnesium sulfate, and then concentrated under reduced pressure. The resulting residue was purified by chromatography, and thus the title compound (480 mg) was obtained as a brown powder.

¹H NMR (300 MHz, DMSO-d₆) δ ppm 4.94 (2H, q, J=8.8 Hz), 6.40 (1H, dd, J=3.3, 2.2 Hz), 6.78 (1H, dd, J=3.3, 2.2 Hz), 7.14-7.22 (1H, m), 7.27-7.37 (1H, m), 7.40(1H, d, J=2.3 Hz), 11.31 (1H, br. s.), 13.60(1H, s).

### Example 39

### 3-[3-Fluoro-4-(2,2,2-trifluoroethoxy)phenyl]-2-thioxo-1,2,3,7-tetrahydro-4H-pyrrolo[2,3-d]pyrimidin-4-one

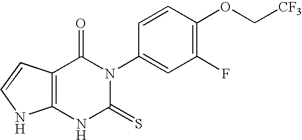

A mixture of ethyl 2-isothiocyanato-1H-pyrrol-3-carboxylate (500 mg) obtained by the method of Reference Example 36, or a method pursuant to thereto, 3-fluoro-4-(2,2,2-trifluoroethoxy)aniline (533 mg) obtained by the method of Reference Example 24, or a method pursuant to thereto, and acetonitrile (20 ml) was heated to reflux for 2 hours. The mixture was ice-cooled, and then a 20% sodium ethoxide-ethanol solution (3.5 ml) and ethanol (3.5 ml) were added thereto. The mixture was stirred for 2 hours at 95° C. The reaction mixture was returned to room temperature, and the solvent was distilled off under reduced pressure. The residue was made basic with a 1 M aqueous solution of sodium hydroxide, and then was washed with a mixed solvent of 25% tetrahydrofuran/diethyl ether. The aqueous layer obtained therefrom was acidified with 1 M hydrochloric acid, and precipitates generated therefrom were collected by filtration. Thus, the title compound (380 mg) was obtained as a pale brown powder.

¹H NMR (300 MHz, DMSO-d₆) δ ppm 4.92 (2H, q, J-8.9 Hz), 6.39 (1H, dd, J=3.3, 2.2 Hz), 6.78 (1H, dd, J=3.2, 2.2 Hz), 7.02 (1H, dt, J=8.7, 1.9 Hz), 7.25 (1H, dd, J=11.8, 2.4 Hz), 7.33 (1H, t, J=9.0 Hz), 11.30 (1H, br. s.), 13.58 (1H, s).

### Example 40

### 3-[3-Methyl-4-(2,2,2-trifluoroethoxy)phenyl]-2-thioxo-1,2,3,7-tetrahydro-4H-pyrrolo[2,3-d]pyrimidin-4-one

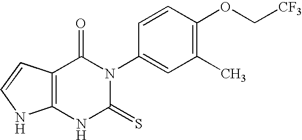

A mixture of ethyl 2-isothiocyanato-1H-pyrrol-3-carboxylate (400 mg) obtained by the method of Reference Example 36, or a method pursuant to thereto, 3-methyl-4-(2,2,2-trifluoroethoxy)aniline (419 mg) obtained by the method of Reference Example 25, or a method pursuant to thereto, and acetonitrile (20 ml) was heated to reflux for 2 hours. The mixture was ice-cooled, and then a 20% sodium ethoxide-ethanol solution (3.5 ml) and ethanol (3.5 ml) were added thereto. The mixture was stirred for one hour at 90° C. The reaction mixture was returned to room temperature, and the solvent was distilled off under reduced pressure. The residue was acidified with 1 M hydrochloric acid, and precipitates generated therefrom were collected by filtration. Thus, the title compound (654 mg) was obtained as a brown powder.

¹H NMR (300 MHz, DMSO-d₆) δ ppm 2.18 (3H, s), 4.81 (2H, q, J=8.9 Hz), 6.38 (1H, dd, J=3.2, 2.2 Hz), 6.77 (1H, dd, J=3.2, 2.2 Hz), 6.93-7.02 (2H, m), 7.09 (1H, d, J=1.5 Hz), 11.26 (1H, br. s.), 13.51 (1H, s).

### Example 41

### 2-Thioxo-3-[4-(2,2,2-trifluoroethoxy)phenyl]-2,3,5,7-tetrahydro-1H-pyrrolo[2,3-d]pyrimidine-4,6-dione

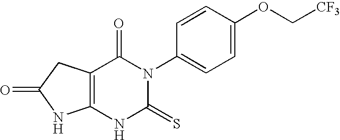

### Example 41a)

Ethyl 3-amino-3-iminopropanoate hydrochloride (3.92 g) obtained by a method described in a published document, Chemical and Pharmaceutical Bulletin (Chem. Pharm. Bull.), Vol. 43, p. 788 (1995), or a method pursuant to thereto, was suspended in ethanol (23 ml), and triethylamine (7.21 ml) was added to the suspension, then the suspension was turned into a clear solution. To the solution was added dropwise isopropyl bromoacetate (3.37 ml). The mixture was stirred for 2 hours at room temperature and diluted with ethyl acetate (30 ml). The white precipitates were filtered off and the filtrate was evaporated under reduced pressure. Acetonitrile (5 ml) and ethyl acetate (50 ml) were added to the resudue, and the precipitated white solid was filtered off. The filtrate was washed with a saturated aqueous solution of sodium hydrogen carbonate (25 ml) and saturated brine, dried over anhydrous magnesium sulfate, and evaporated under reduced pressure. The residue, yellow oily substance and white solid, was recrystallized from 20% ethyl acetate/hexane (200 ml) to give 1-ethyl 4-(1-methylethyl) 2-(diaminomethylidene)butanedioate (2.83 g) as white cotton-like needles.

¹H NMR (300 MHz, DMSO-d₆) δ ppm 1.07 (3H, t, J=7.0 Hz), 1.16 (6H, d, J=6.3 Hz), 3.01 (2H, s), 3.86 (2H, q, J=7.0 Hz), 4.82 (1H, spt, J=6.3 Hz), 5.73 (2H, br. s.), 7.02 (2H, br. s.).

### Example 41b)

1-Ethyl 4-(1-methylethyl) 2-(diaminomethylidene)butanedioate (12.8 g) obtained by the method of Example (41a), or a method pursuant to thereto, and 1-isothiocyanato-4-(2,2,2-trifluoroethoxy)benzene (10.0 g) were dissolved in acetonitrile (56 ml) and the solution was stirred at 50° C. for 10 hours. The reaction mixture was cooled to room temperature and concentrated under reduced pressure to give an orange-colored oily residue. The residue was dissolved in ethanol (56 ml) and a 20% sodium ethoxide-ethanol solution (36.5 g) was added to the solution. The mixture was stirred for 20 minutes at room temperature. Then, the reaction mixture was poured into 0.5 M hydrochloric acid (220 ml) with ice cooling. Green precipitates were collected by filtration and washed with water. After drying, the precipitates were washed with 50% ethyl acetate/hexane to give the titled compound, 2-thioxo-3-[4-(2,2,2-trifluoroethoxy)phenyl]-2,3,5,7-tetrahydro-1H-pyrrolo[2,3-d]pyrimid ine-4,6-dione (12.7 g) as a white powder.

¹H NMR (300 MHz, DMSO-d₆) δ ppm 3.33 (2H, s), 4.81 (2H, q, J=8.9 Hz), 7.10 (4H, s), 10.89 (1H, br. s.), 13.69 (1H, br. s.).

### Example 42

### 2-Thioxo-3-[4-(3,3,3-trifluoropropyl)phenyl]-1,2,3,7-tetrahydro-4H-pyrrolo[2,

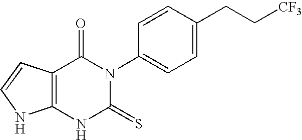

A mixture of 4-(3,3,3-trifluoropropyl)aniline hydrochloride (1.13 g) obtained in Reference Example 27 and a 5% aqueous solution of sodium hydrogen carbonate was extracted with ethyl acetate. The organic layer was washed with water and saturated brine, dried over anhydrous sodium sulfate, and then concentrated under reduced pressure, to obtain 4-(3,3,3-trifluoropropyl)aniline (0.928 g) as a yellow oily substance. A mixture of 4-(3,3,3-trifluoropropyl)aniline (0.928 g), ethyl 2-isothiocyanato-1H-pyrrol-3-carboxylate (0.876 g) obtained by the method of Reference Example 36, or a method pursuant to thereto, and acetonitrile (20 ml) was stirred for one hour at 80° C. A 20% sodium ethoxide-ethanol solution (5.9 ml) was added to the mixture, and the resulting mixture was stirred for another one hour at 80° C., and then was concentrated under reduced pressure. The residue was diluted with water, and then the pH was adjusted to about 6 with 1 M hydrochloric acid. A solid precipitated therefrom was collected by filtration, washed with water and diisopropyl ether, and then dried, and thus the title compound (1.45 g) was obtained as a pale brown solid.

¹H NMR (300 MHz, DMSO-d₆) δ ppm 2.56-2.75 (2H, m), 2.84-2.92 (2H, m), 6.38 (1H, dd, J=3.4, 1.9 Hz), 6.77 (1H, dd, J=3.4, 2.3 Hz), 7.09 (2H, d, J=8.3 Hz), 7.36 (2H, d, J=8.3 Hz), 11.26 (1H, br. s.), 13.51 (1H, br. s.).

### Example 43

### 3-[4-(2-Cyclopropylethyl)phenyl]-2-thioxo-1,2,3,7-tetrahydro-4H-pyrrolo[2,3-d]pyrimidin-4-one

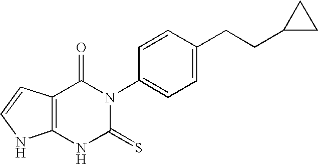

A mixture of 4-(2-cyclopropylethyl)aniline hydrochloride (0.593 g) obtained in Reference Example 28 and a 5% aqueous solution of sodium hydrogen carbonate was extracted with ethyl acetate. The organic layer was washed with water and saturated brine, dried over anhydrous sodium sulfate, and then concentrated under reduced pressure, to obtain 4-(2-cyclopropylethyl)aniline (0.477 g) as a yellow oily substance. A mixture of 4-(2-cyclopropylethyl)aniline (0.477 g) and ethyl 2-isothiocyanato-1H-pyrrol-3-carboxylate (0.526 g) obtained by the method of Reference Example 36, or a method pursuant to thereto, and acetonitrile (20 ml) was stirred for one hour at 80° C. A 20% sodium ethoxide-ethanol solution (3.5 ml) was added to the mixture, and the resulting mixture was stirred for one hour at 80° C., and then was concentrated under reduced pressure. The residue was diluted with water, and then the pH was adjusted to about 6 with 1 M hydrochloric acid. A solid precipitated therefrom was collected by filtration, washed with water and diisopropyl ether, and then dried, and thus the title compound (0.791 g) was obtained as a pale brown solid.

¹H NMR (300 MHz, DMSO-d₆) δ ppm 0.05-0.12 (2H, m), 0.38-0.46 (2H, m), 0.67-0.82 (1H, m), 1.47-1.58 (2H, m), 2.67-2.76 (2H, m), 6.38 (1H, dd, J=3.0, 1.9 Hz), 6.77 (1H, dd, J=3.0, 2.3 Hz), 7.04 (2H, d, J=8.3 Hz), 7.26 (2H, d, J=8.3 Hz), 11.26 (1H, br. s.), 13.43 (1 br. s.).

### Example 44

### 3-[4-(2,2-Difluoroethoxy)phenyl]-2-thioxo-1,2,3,7-tetrahydro-4H-pyrrolo[2,3-d]pyrimidin-4-one

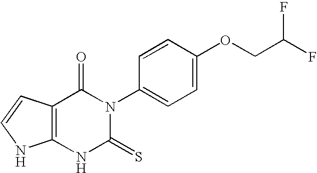

A mixture of 4-(2,2-difluoroethoxy)aniline (1.0 g) obtained in Reference Example 29 and ethyl 2-isothiocyanato-1H-pyrrol-3-carboxylate (0.952 g) obtained by the method of Reference Example 36, or a method pursuant to thereto, and acetonitrile (20 ml) was heated to reflux for 2 hours, and then was cooled in an ice water bath. A 20% sodium ethoxide-ethanol solution (7 ml) was added to the mixture, and the resulting mixture was heated to reflux for 4 hours, and then was concentrated under reduced pressure. The residue was diluted with water, and then the pH was adjusted to about 4 with 1 M hydrochloric acid. A solid precipitated therefrom was collected by filtration, washed with water, and then dissolved in a 1 M aqueous solution of sodium hydroxide. The aqueous solution was washed with a mixed solvent of diethyl ether and tetrahydrofuran, and then the pH was adjusted to about 4 with 1 M hydrochloric acid. A solid precipitated therefrom was collected by filtration, washed with water and diisopropyl ether, and then dried, and thus the title compound (1.32 g) was obtained as a pale brown solid.

¹H NMR (300 MHz, DMSO-d₆) δ ppm 4.37 (2H, td, J=14.7, 3.5 Hz), 6.43 (1H, tt, J=54.5, 3.5 Hz), 6.38 (1H, dd, J=3.2, 1.9 Hz), 6.77 (1H, dd, J=3.2, 2.3 Hz), 7.05 (2H, d, J=9.2 Hz), 7.10 (2H, d, J=9.2 Hz), 11.26 (1H, br. s.), 13.50 (1H, br. s.).

### Example 45

### 3-[4-(2,2,3,3,3-Pentafluoropropoxy)phenyl]-2-thioxo-1,2,3,7-tetrahydro-4H-pyrrolo[2,3-d]pyrimidin-4-one

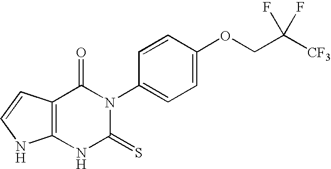

A mixture of 4-(2,2,3,3,3-pentafluoropropoxy)aniline (1.0 g) obtained in Reference Example 30, ethyl 2-isothiocyanato-1H-pyrrol-3-carboxylate (1.33 g) obtained by the method of Reference Example 36, or a method pursuant to thereto, and acetonitrile (20 ml) was heated to reflux for 2 hours, and then was cooled in an ice water bath. A 20% sodium ethoxide-ethanol solution (7 ml) was added to the mixture, and the resulting mixture was further heated to reflux for 4 hours, and then was concentrated under reduced pressure. The residue was diluted with water, and then the pH was adjusted to about 4 with 1 M hydrochloric acid. A solid precipitated therefrom was collected by filtration, washed with water, and then dissolved in a 1 M aqueous solution of sodium hydroxide. The aqueous solution was washed with a mixed solvent of diethyl ether/tetrahydrofuran, and then the pH was adjusted to about 4 with 1 M hydrochloric acid. A solid precipitated therefrom was collected by filtration, washed with water and diisopropyl ether, and then dried, and thus the title compound (1.75 g) was obtained as a pale brown solid.

¹H NMR (300 MHz, DMSO-d₆) δ ppm 4.89 (2H, t, J=13.1 Hz), 6.39 (1H, dd, J=3.6, 0.9 Hz), 6.77 (1H, dd, J=3.2, 1.3 Hz), 7.12 (2H, d, J=9.1 Hz), 7.12 (2H, d, J=9.1 Hz), 11.26 (1H, br. s.), 13.52 (1H, s).

### Example 46

### 2-Thioxo-3-[4-(2,2,2-trifluoroethoxy)phenyl]-2,3,5,7-tetrahydro-1H-pyrrolo[2,3-d]pyrimidine-4,6-dione

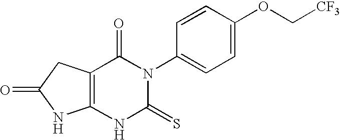

### Example 46a

Ethyl 3-amino-3-iminopropanoate hydrochloride (10 g) obtained by a method described in a published document, Chemical and Pharmaceutical Bulletin (Chem. Pharm. Bull.), Vol. 43, p. 788 (1995), or a method pursuant to thereto, was suspended in ethanol (60 ml), and a 20% sodium ethoxide-ethanol solution (20.4 g) was added to the suspension. The mixture was stirred for 5 minutes and precipitates were filtered through Celite. The filtrate was concentrated under reduced pressure to obtain a brown oily residue. This oily residue was dissolved in acetonitrile (120 ml) and triethylamine (8.32 ml) was added to the solution. Then, ethyl bromoacetate (6.66 g) was added dropwise to the solution and the mixture was stirred for one hour at room temperature. The reaction mixture was concentrated under reduced pressure while maintained at 30° C. or below and the resultant brown crude product was dissolved in acetonitrile (20 ml). Ethyl acetate (150 ml) was added to the solution and precipitates were filtered. The filtrate was diluted with ethyl acetate (100 ml), and the dilution was washed with a saturated aqueous solution of sodium hydrogen carbonate (60 ml), and saturated brine, dried over anhydrous magnesium sulfate, and then concentrated under reduced pressure while maintained at 30° C. or below to give a white solid.

### Example 46b

The white solid obtained in Example 46a and 1-isothiocyanato-4-(2,2,2-trifluoroethoxy)benzene (4.66 g) were dissolved in acetonitrile (100 ml) and the solution was heated to reflux for one hour. The reaction mixture was cooled to room temperature and concentrated under reduced pressure to give an orange-colored oily residue. This oily residue was purified by chromatography to give a yellow amorphous substance (6.4 g).

MS(ESI+):450(M+H).

### Example 46c

The yellow amorphous substance (6.4 g) obtained in Example (46b) was dissolved in ethanol (70 ml) and a 20% sodium ethoxide-ethanol solution (12.1 g) was added to the solution. The mixture was stirred for 20 minutes at room temperature. Then, the reaction mixture was poured into 0.2 M hydrochloric acid (200 ml) with ice cooling. Green precipitates were collected by filtration and washed with water. These precipitates were dissolved in tetrahydrofuran (30 ml) and the solution was diluted with ethyl acetate (150 ml), washed with saturated brine, dried over anhydrous magnesium sulfate, and concentrated under reduced pressure to give a dark green solid, which was washed with diethyl ether to give the title compound, 2-thioxo-3-[4-(2,2,2-trifluoroethoxy)phenyl]-2,3,5,7-tetrahydro-1H-pyrrolo[2,3-d]pyrimidine-4,6-dione (4.2 g), as a gray solid.

¹H NMR (300 MHz, DMSO-d₆) δ ppm 3.32 (2H, s), 4.81 (2H, q, J=8.9 Hz), 7.10 (4H, s), 10.85 (1H, br. s.), 13.66 (1H, br. s.).

### Example 47

### 3-[4-(2,2,3,3,3-Pentafluoropropoxy)phenyl]-2-thioxo-2,3,5,7-tetrahydro-1H-pyrrolo[2,3-d]pyrimidine-4,6-dione

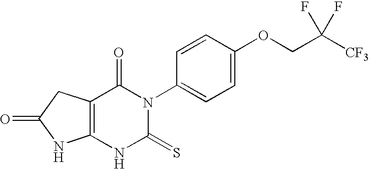

### Example 47a

A mixture of the white solid (7.24 g) obtained by the method of Example (46a), or a method pursuant to thereto, 1-isothiocyanato-4-(2,2,3,3,3-pentafluoropropoxy)benzene (4.82 g) obtained by the method of Reference Example 51, or a method pursuant to thereto, and acetonitrile (60 ml) was heated to reflux for one hour, and then was concentrated under reduced pressure. The residue was purified by chromatography, and thus a brown oily substance (6.17 g) was obtained.

### Example 47b

To a solution of the brown oily substance (6.17 g) obtained in Example (47a) in ethanol (35 ml), a 20% sodium ethoxide-ethanol solution (6.76 g) was added dropwise over 5 minutes. The mixture was stirred for 30 minutes, and then added dropwise to 0.5 M hydrochloric acid (100 ml) in an ice water bath. A solid precipitated therefrom was collected by filtration, washed with water and then dried. The solid was suspended in ethyl acetate, and diethyl ether was added thereto. The solid was collected by filtration, washed with diethyl ether and then dried. Thus, the title compound, 3-[4-(2,2,3,3,3-pentafluoropropoxy)phenyl]-2-thioxo-2,3,5,7-tetrahydro-1H-pyrrolo[2,3-d]pyrimidine-4,6-dione (4.26 g), was obtained as a pale gray solid.

¹H NMR (300 MHz, DMSO-d₆) δ ppm 3.32 (2H, s), 4.88 (2H, t, J=13.4 Hz), 7.11 (4H, s), 10.86(1H, br. s.), 13.68(1H, br. s.).

### Example 48

### 3-[4-(Cyclopropylmethoxy)phenyl]-2-thioxo-2,3,5,7-tetrahydro-1H-pyrrolo[2,3-d]pyrimidine-4,6-dione

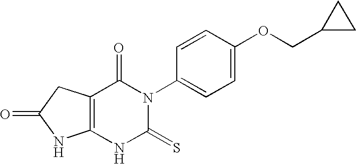

The white solid (3.24 g) obtained by the method of Example (46a), or a method pursuant to thereto, and 1-(cyclopropylmethoxy)-4-isothiocyanatobenzene (930 mg) obtained by the method of Reference Example 52, or a method pursuant to thereto, were dissolved in acetonitrile (30 ml). The resulting mixture was heated to reflux for one hour. The reaction mixture was returned to room temperature, and then was concentrated under reduced pressure. An orange-colored oily substance obtained therefrom was purified by chromatography, to obtain a yellow oily substance (1.59 g). This oily substance (1.55 g) was dissolved in ethanol (18 ml), and a 20% sodium ethoxide-ethanol solution (3.13 g) was added thereto, and the resulting mixture was stirred for 30 minutes at room temperature. Subsequently, the reaction solution was poured into 0.2 M hydrochloric acid (75 ml) under ice cooling, and precipitates generated therefrom were collected by filtration, washed with water, and then dissolved in tetrahydrofuran (50 ml). The solution was diluted with ethyl acetate (200 ml), and the dilution was washed with saturated brine, dried over anhydrous magnesium sulfate, and concentrated under reduced pressure, to obtain a brown solid. This solid was washed with a mixed solvent of about 10% ethyl acetate/diethyl ether, and thus the title compound (965 mg) was obtained as a gray solid.

¹H NMR (300 MHz, DMSO-d₆) δ ppm 0.30-0.38 (2H, m), 0.54-0.64 (2H, m), 1.20-1.33 (1H, m), 3.32 (2H, s), 3.84 (2H, d, J=6.8 Hz), 6.95 (2H, d, J=9.1 Hz), 7.01 (2H, d, J=9.1 Hz), 10.82 (1H, br. s.), 13.62 (1H, br. s.).

### Example 49

### 3-[3-Fluoro-4-(2,2,2-trifluoroethoxy)phenyl]-2-thioxo-2,3,5,7-tetrahydro-1H-pyrrolo[2,3-d]pyrimidine-4,6-dione

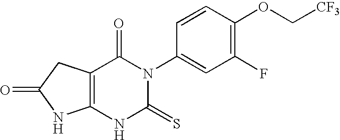

The white solid (7.78 g) obtained by the method of Example (46a), or a method pursuant to thereto, and 2-fluoro-4-isothiocyanato-1-(2,2,2-trifluoroethoxy)benzene (3.01 g) obtained by the method of Reference Example 39, or a method pursuant to thereto, were dissolved in acetonitrile (100 ml). The resulting mixture was heated to reflux for one hour. The reaction mixture was returned to room temperature, and then was concentrated under reduced pressure. An orange-colored oily substance obtained therefrom was purified by chromatography, and thus a yellow oily substance (2.96 g) was obtained. This oily substance was dissolved in ethanol (30 ml), and a 20% sodium ethoxide-ethanol solution (5.39 g) was added thereto. The mixture was stirred for 30 minutes at room temperature. Subsequently, the reaction solution was poured into 0.2 M hydrochloric acid (100 ml) under ice cooling, and precipitates generated therefrom were collected by filtration, washed with water, and then dissolved in tetrahydrofuran (20 ml). The solution was diluted with ethyl acetate (100 ml), and the dilution was washed with saturated brine, dried over anhydrous magnesium sulfate, and then concentrated under reduced pressure, to obtain a brown crude product. This crude product was purified by chromatography, and thus a brown oily substance was obtained. This oily substance was dissolved in ethyl acetate, and hexane was added thereto. Precipitates generated therefrom were collected by filtration and washed with diethyl ether, and thus the title compound (730 mg) was obtained as a yellowish white powder.

¹H NMR (300 MHz, DMSO-d₆) δ ppm 3.33 (2H, s), 4.91 (2H, q, J=8.8 Hz), 6.96-7.05 (1H, m), 7.22 (1H, dd, J=11.7, 2.3 Hz), 7.34 (1H, dd, J=9.4, 8.7 Hz), 10.91 (1H, br. s.), 13.76 (1H, br. s.).

### Example 50

### 3-[4-(Cyclopropylmethoxy)-3-fluorophenyl]-2-sulfanyl-3,5-dihydro-4H-pyrrolo[3,2-d]pyrimidin-4-one

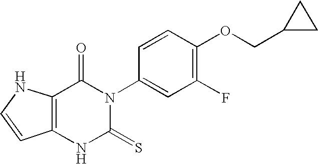

A mixture of ethyl 3-isothiocyanato-1H-pyrrol-2-carboxylate (1.0 g) obtained by the method of Reference Example 31, or a method pursuant to thereto, 4-(cyclopropylmethoxy)-3-fluoroaniline (924 mg) obtained by the method of Reference Example 38, or a method pursuant to thereto, and acetonitrile (20 ml) was heated to reflux for one hour. The mixture was ice-cooled, and then a suspension of potassium tert-butoxide (2.36 g) in ethanol (10 ml) was added thereto. The mixture was stirred for 2 hours at 90° C. The reaction mixture was returned to room temperature, and the solvent was distilled off under reduced pressure. The residue was acidified with 1 M hydrochloric acid. Then, precipitates generated therefrom were collected by filtration, washed with a mixed solvent of 50% ethyl acetate/hexane, and dried under reduced pressure. Thus, the title compound (1.40 g) was obtained as a pale yellow powder.

¹H NMR (300 MHz, DMSO-d₆) δ ppm 0.32-0.42 (2H, m), 0.57-0.66 (2H, m), 1.21-1.38 (1H, m), 3.94 (2H, d, J=7.2 Hz), 6.00-6.03 (1H, m), 6.95 (1H, d, J=8.7 Hz), 7.12-7.18 (1H, m), 7.18-7.21 (1H, m), 7.32-7.38 (1H, m), 12.31 (1H, br. s.), 12.94 (1H, s).

### Example 51

### N-(2-cyanoethyl)-2-{[3-(4-ethoxyphenyl)-4-oxo-4,5-dihydro-3H-pyrrolo[3,2-d]pyrimidin-2-yl]sulfanyl}acetamide

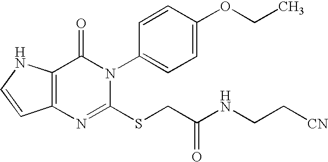

A mixture of 3-(4-ethoxyphenyl)-2-thioxo-1,2,3,5-tetrahydro-4H-pyrrolo[3,2-d]pyrimidin-4-one (575 mg) obtained by the method of Example 1, or a method pursuant to thereto, 2-chloro-N-(2-cyanoethyl)acetamide (293 mg), obtained by the method of Reference Example 40, or a method pursuant to thereto, triethylamine (418 μl) and acetonitrile (20 ml) was heated to reflux for one hour. The reaction mixture was returned to room temperature, and then silica gel was added thereto. The mixture was concentrated under reduced pressure, and a crude product obtained therefrom was purified by chromatography, and thus the title compound (759 mg) was obtained as a white solid.

¹H NMR (400 MHz, DMSO-d₆) δ ppm 1.38 (3H, t, J=7.0 Hz), 2.61 (2H, t, J=6.6 Hz), 3.28 (2H, td, J=6.6, 5.8 Hz), 3.81 (2H, s), 4.11 (2H, q, J=7.0 Hz), 6.34 (1H, d, J=2.7 Hz), 7.07 (2H, d, J=8.8 Hz), 7.28 (2H, d, J=8.8 Hz), 7.39 (1H, d, J=2.7 Hz), 8.52(1H, t, J=5.7 Hz), 12.15 (1H, s).

### Example 52

### N-(2-cyanoethyl)-2-{[3-(4-ethoxyphenyl)-4-oxo-4,5-dihydro-3H-pyrrolo[3,2-d]pyrimidin-2-yl]sulfanyl}-N-methylacetamide

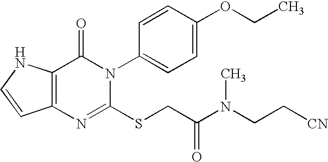

A mixture of 3-(4-ethoxyphenyl)-2-thioxo-1,2,3,5-tetrahydro-4H-pyrrolo[3,2-d]pyrimidin-4-one (144 mg) obtained by the method of Example 1, or a method pursuant to thereto, 2-chloro-N-(2-cyanoethyl)-N-methylacetamide (151 mg) obtained by the method of Example 41, or a method pursuant to thereto, triethylamine (105 μl) and acetonitrile (10 ml) was heated to reflux for one hour. The reaction mixture was returned to room temperature, and then silica gel was added thereto. The mixture was concentrated under reduced pressure, and a crude product obtained therefrom was purified by chromatography, and thus the title compound (151 mg) was obtained as a white solid.

¹H NMR (400 MHz, DMSO-d₆) δ ppm 1.38 (3H, t, J=7.0 Hz), 2.67 (1.3 H, t, J=6.7 Hz), 2.85 (1.1 H, s), 2.97 (0.7H, t, J=6.7 Hz), 3.17 (1.9H, s), 3.53 (1.3H, t, J=6.7 Hz), 3.78 (0.7H, t, J=6.7 Hz), 4.06 (1.3H, s), 4.12 (2H, q, J=7.0 Hz), 4.09 (0.7H, s), 6.30-6.38 (1H, m), 7.07 (2H, d, J=8.8 Hz), 7.26 (2H, d, J=8.8 Hz), 7.35-7.43 (1H, m), 12.14 (1H, br. s.).

### Example 53

### N-(2-cyanoethyl)-2-{[3-(4-ethoxyphenyl)-4-oxo-4,5-dihydro-3H-pyrrolo[3,2-d]pyrimidin-2-yl]sulfanyl}propanamide

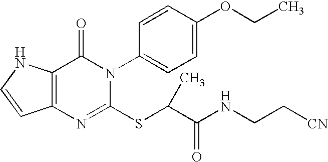

A mixture of 3-(4-ethoxyphenyl)-2-thioxo-1,2,3,5-tetrahydro-4H-pyrrolo[3,2-d]pyrimidin-4-one (100 mg) obtained by the method of Example 1, or a method pursuant to thereto, 2-chloro-N-(2-cyanoethyl)propanamide (56 mg) obtained by the method of Reference Example 42, or a method pursuant to thereto, triethylamine (96 μl) and N,N-dimethylformamide (2 ml) was heated to 110° C., and was stirred for 12 hours. The reaction mixture was returned to room temperature, and then was concentrated under reduced pressure. The residue was dissolved in acetonitrile, and silica gel was added thereto. The resulting mixture was concentrated again under reduced pressure. The crude product obtained therefrom was purified by chromatography, and thus the title compound (98 mg) was obtained as a white solid.

¹H NMR (400 MHz, DMSO-d₆) δ ppm 1.37 (3H, t, J=7.0 Hz), 1.42 (3H, d, J=7.1 Hz), 2.61 (2H, t, J=6.4 Hz), 3.27 (2H, td, J=6.4, 5.9 Hz), 4.11 (2H, q, J=7.0 Hz), 4.41 (1H, q, J=7.1 Hz), 6.36 (1H, d, J=2.9 Hz), 7.01-7.09 (2H, m), 7.18-7.29 (2H, m), 7.40 (1H, d, J=2.9 Hz), 8.60 (1H, t, J=5.9 Hz), 12.16 (1H, s).

### Example 54

### 3-{[3-(4-Ethoxyphenyl)-4-oxo-4,5-dihydro-3H-pyrrolo[3,2-d]pyrimidin-2-yl]sulfanyl}propanoic acid

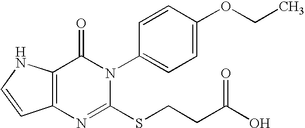

A mixture of 3-(4-ethoxyphenyl)-2-thioxo-1,2,3,5-tetrahydro-4H-pyrrolo[3,2-d]pyrimidin-4-one (100 mg) obtained by the method of Example 1, or a method pursuant to thereto, 3-iodopropanoic acid (69 mg), triethylamine (144 μl) and N,N-dimethylformamide (3 ml) was heated to 110° C., and was stirred for 12 hours. Subsequently, 3-iodopropanoic acid (140 mg) and triethylamine (288 μl) were further added thereto, and the resulting mixture was stirred for 3 hours at 110° C. 3-iodopropanoic acid (69 mg) and triethylamine (144 μl) were further added thereto, and the resulting mixture was stirred for 3 hours at 110° C. Subsequently, the reaction mixture was concentrated under reduced pressure, and water (5 ml) was added to the residue. The mixture was acidified with 1 M hydrochloric acid, salted out, and extracted with ethyl acetate. The organic layer was washed with saturated brine, dried over anhydrous magnesium sulfate, and then concentrated under reduced pressure, to obtain a brown crude product. The crude product was purified by chromatography, and thus the title compound (50 mg) was obtained.

MS(ESI+):360(M+H).

### Example 55

### N-(2-cyanoethyl)-3-{[3-(4-ethoxyphenyl)-4-oxo-4,5-dihydro-3H-pyrrolo[3,2-d]pyrimidin-2-yl]sulfanyl}propanamide

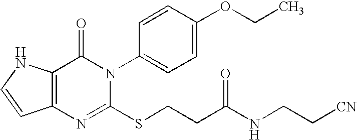

A mixture of 3-{[3 -(4-ethoxyphenyl)-4-oxo-4,5-dihydro-3H-pyrrolo[3,2-d]pyrimidin-2-yl]sulfanyl}propanoic acid (48 mg) obtained in Example 54, 1-ethyl-3-(3-dimethylaminopropyl)carbodiimide hydrochloride (40 mg), 1-hydroxybenzotriazole (22 mg), 3-aminopropanenitrile (50 mg) and acetonitrile (3 ml) was stirred overnight at room temperature, and then was diluted with ethyl acetate (60 ml). The dilution was washed sequentially with 0.2 M hydrochloric acid, a saturated aqueous solution of sodium hydrogen carbonate and saturated brine, dried over anhydrous magnesium sulfate, and then concentrated under reduced pressure, to obtain a white solid. This solid was purified by chromatography, and thus the title compound (35 mg) was obtained as a white solid.

¹H NMR (400 MHz, DMSO-d₆) δ ppm 1.37 (3H, t, J=7.0 Hz), 2.44-2.54 (2H, m), 2.60 (2H, t, J=6.4 Hz), 3.21 (2H, t, J=6.8 Hz), 3.25 (2H, td, J=6.4, 5.6 Hz), 4.10 (2H, q, J=7.0 Hz), 6.37 (1H, d, J=2.9 Hz), 7.03 (2H, d, J=8.8 Hz), 7.22 (2H, d, J=8.8 Hz), 7.39 (1H, d, J=2.9 Hz), 8.29 (1H, t, J=5.6 Hz), 12.13 (1H, s).

### Example 56

N-(2-cyanoethyl)-1-{[3 -(4-ethoxyphenyl)-4-oxo-4,5-dihydro-3H-pyrrolo[3,2-d]pyrimidin-2-yl]sulfanyl}methanesulfonamide

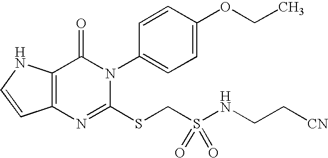

A mixture of 3-(4-ethoxyphenyl)-2-thioxo-1,2,3,5-tetrahydro-4H-pyrrolo[3,2-d]pyrimidin-4-one (100 mg) obtained by the method of Example 1, or a method pursuant to thereto, 1-chloro-N-(2-cyanoethyl)methanesulfonamide (127 mg) obtained by the method of Reference Example 43, or a method pursuant to thereto, triethylamine (145 μl and N,N-dimethylformamide (2 ml) was heated to 120° C., and was stirred for 24 hours. The mixture was returned to room temperature, and then was concentrated under reduced pressure. Water (20 ml) was added to the residue, and the mixture was extracted with a mixed solvent of 30% tetrahydrofuran/ethyl acetate. The organic layer was washed with saturated brine, dried over anhydrous magnesium sulfate, and then concentrated under reduced pressure, to obtain a brown solid. This solid was purified by chromatography, and thus the title compound (29 mg) was obtained as white crystals.

¹H NMR (400 MHz, DMSO-d₆) δ ppm 1.38 (3H, t, J=7.0 Hz), 2.61 (2H, t, J=6.5 Hz), 3.22 (2H, t, J=6.5 Hz), 4.12 (2H, q, J=7.0 Hz), 4.87 (2H, s), 6.39 (1H, d, J=2.7 Hz), 7.09 (2H, d, J=8.8 Hz), 7.32 (2H, d, J=8.8 Hz), 7.44 (1H, d, J=2.7 Hz), 7.84 (1H, br. s.), 12.25 (1H, br. s.).

### Example 57

### 3-[(2-{[3-(4-Ethoxyphenyl)-4-oxo-4,5-dihydro-3H-pyrrolo[3,2-d]pyrimidin-2-yl]sulfanyl}}ethyl)amino]propanenitrile

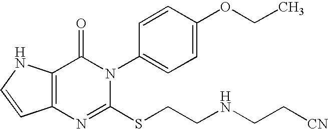

A mixture of 3-(4-ethoxyphenyl)-2-thioxo-1,2,3,5-tetrahydro-4H-pyrrolo[3,2-d]pyrimidin-4-one (144 mg) obtained by the method of Example 1, or a method pursuant to thereto, chloroacetaldehyde (45% aqueous solution, 87 mg), triethylamine (105 μl), tetrahydrofuran (1 ml) and acetonitrile (2 ml) was heated to 100° C., and was stirred for 2 hours. The mixture was returned to room temperature, and was diluted with ethyl acetate (80 ml). The dilution was washed sequentially with water and saturated brine, dried over anhydrous magnesium sulfate, and then concentrated under reduced pressure, to obtain a brown oily substance. This oily substance was dissolved in acetonitrile (5 ml), and 3-aminopropanenitrile (175 mg) and sodium triacetoxyhydroborate (159 mg) were added thereto. The mixture was stirred for 12 hours at room temperature. Methanol (1 ml) was added to the reaction solution, and then the mixture was concentrated under reduced pressure. To the resulting residue, a saturated aqueous solution of sodium hydrogen carbonate (10 ml) was added, and the mixture was extracted with ethyl acetate. The organic layer was washed with saturated brine, dried over anhydrous magnesium sulfate, and then concentrated under reduced pressure, to obtain a brown residue. This residue was purified by chromatography, and thus the title compound (88 mg) was obtained as a yellowish white solid.

¹H NMR (400 MHz, DMSO-d₆) δ ppm 1.37 (3H, t, J=7.0 Hz), 2.26 (1H, br. s.), 2.57 (2H, t, J=6.6 Hz), 2.72-2.82 (4H, m), 3.13 (2H, t, J=6.8 Hz), 4.10 (2H, q, J=7.0 Hz), 6.34 (1H, dd, J=2.7, 1.7 Hz), 7.04 (2H, d, J=8.8 Hz), 7.24 (2H, d, J=8.8 Hz), 7.39 (1H, dd, J=3.2, 2.7 Hz), 12.12 (1H, br. s.).

### Example 58

### 3-(4-Ethoxyphenyl)-2-[(1H-imidazol-2-ylmethyl)sulfanyl]-3,5-dihydro-4H-pyrrolo[3,2-d]pyrimidin-4-one

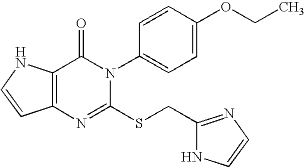

A mixture of 3-(4-ethoxyphenyl)-2-thioxo-1,2,3,5-tetrahydro-4H-pyrrolo[3,2-d]pyrimidin-4-one (144 mg) obtained by the method of Example 1, or a method pursuant to thereto, 2-(chloromethyl)-1H-imidazole hydrochloride (116 mg), triethylamine (145 μl), sodium iodide (37.5 mg) and N,N-dimethylformamide (3 ml) was heated to 120° C., and was stirred for 12 hours. The mixture was returned to room temperature, and then a saturated aqueous solution of sodium hydrogen carbonate (6 ml), water (10 ml) and tetrahydrofuran (10 ml) were added to the mixture. The resulting mixture was extracted with a mixed solvent of 30% tetrahydrofuran/ethyl acetate, and the organic layer was washed with saturated brine, dried over anhydrous magnesium sulfate, and then concentrated under reduced pressure, to obtain a brown solid. This solid was purified by chromatography, and thus the title compound (53 mg) was obtained as a yellowish white solid. Furthermore, 3-(4-ethoxyphenyl)-2-({[1-(1H-imidazol-2-ylmethyl)-1H-imidazol-2-yl]methyl}sulfanyl)-3,5-dihydro-4H-pyrrolo[3,2-d]pyrimidin-4-one of Example 59 was also obtained.

¹H NMR (400 MHz, DMSO-d₆) δ ppm 1.36 (3H, t, J=7.0 Hz), 4.09 (2H, q, J=7.0 Hz), 4.35 (2H, s), 6.41 (1H, dd, J=2.9, 1.9 Hz), 6.91 (2H, s), 7.04 (2H, d, J=8.8 Hz), 7.26 (2H, d, J=8.8 Hz), 7.41 (1H, t, J=2.9 Hz), 12.02 (1H, br. s.), 12.17 (1H, br. s.)

### Example 59

### 3-(4-Ethoxyphenyl)-2-({[1-(1H-imidazol-2-ylmethyl)-1H-imidazol-2-yl]methyl}sulfanyl)-3,5-dihydro-4H-pyrrolo[3,2-d]pyrimidin-4-one

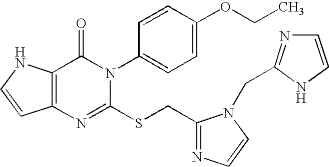

A mixture of 3-(4-ethoxyphenyl)-2-thioxo-1,2,3,5-tetrahydro-4H-pyrrolo[3,2-d]pyrimidin-4-one (144 mg) obtained by the method of Example 1, or a method pursuant to thereto, 2-(chloromethyl)-1H-imidazole hydrochloride (116 mg), triethylamine (145 μl), sodium iodide (37.5 mg) and N,N-dimethylformamide (3 ml) was heated to 120° C., and was stirred for 12 hours. The mixture was returned to room temperature, and then a saturated aqueous solution of sodium hydrogen carbonate (6 ml), water (10 ml) and tetrahydrofuran (10 ml) were added to the mixture. The resulting mixture was extracted with a mixed solvent of 30% tetrahydrofuran/ethyl acetate, and the organic layer was washed with saturated brine, dried over anhydrous magnesium sulfate, and then concentrated under reduced pressure, to obtain a brown solid. This solid was purified by chromatography, and thus the title compound (71 mg) was obtained as a yellowish white solid. Furthermore, 3-(4-ethoxyphenyl)-2-[(1H-imidazol-2-ylmethyl)sulfanyl]-3,5-dihydro-4H-pyrrolo[3,2-d]pyrimidin-4-one of Example 58 was also obtained.

¹H NMR (400 MHz, DMSO-d₆) δ ppm 1.36 (3H, t, J=7.0 Hz), 4.09 (2H, q, J=7.0 Hz), 4.46 (2H, s), 5.25 (2H, s), 6.40 (1H, dd, J=2.9, 1.9 Hz), 6.76 (1H, d, J=1.0 Hz), 6.98 (2H, br. s.), 7.04 (2H, d, J=8.8 Hz), 7.07 (1H, d, J=1.0 Hz), 7.26 (2H, d, J=8.8 Hz), 7.41 (1H, t, J=2.9 Hz), 12.13 (1H, br. s.), 12.17 (1H, br. s.).

### Example 60

### N-(2-cyanoethyl)-4-{[3-(4-ethoxyphenyl)-4-oxo-4,5-dihydro-3H-pyrrolo[3,2-d]pyrimidin-2-yl]sulfanyl}butanamide

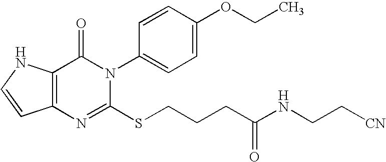

A mixture of 3-(4-ethoxyphenyl)-2-thioxo-1,2,3,5-tetrahydro-4H-pyrrolo[3,2-d]pyrimidin-4-one (400 mg) obtained by the method of Example 1, or a method pursuant to thereto, 4-bromo-N-(2-cyanoethyl)butanamide (336 mg) obtained by the method of Reference Example 49, or a method pursuant to thereto, triethylamine (390 μl), sodium iodide (210 mg) and N,N-dimethylformamide (10 ml) was heated to 120° C., and was stirred for 20 hours. The reaction mixture was returned to room temperature, and was diluted with ethyl acetate (200 ml). This dilution was washed with water and saturated brine, dried over anhydrous magnesium sulfate, and then concentrated under reduced pressure. The resulting residue was purified by chromatography, and thus the title compound (310 mg) was obtained as a white solid.

¹H NMR (400 MHz, DMSO-d₆) δ ppm 1.37 (3H, t, J=7.0 Hz), 1.82 (2H, tt, J=7.3, 7.1 Hz), 2.17 (2H, t, J=7.3 Hz), 2.61 (2H, t, J=6.6 Hz), 3.04 (2H, t, J=7.1 Hz), 3.24 (2H, td, J=6.4, 5.8 Hz), 4.10 (2H, q, J=7.0 Hz), 6.35 (1H, d, J=2.9 Hz), 7.04 (2H, d, J=8.8 Hz), 7.24 (2H, d, J=8.8 Hz), 7.38 (1H, d, J=2.9 Hz), 8.22 (1H, t, J=5.7 Hz), 12.12 (1H, s).

### Example 61

### 3-Cyano-N-(2-{[3-(4-ethoxyphenyl)-4-oxo-4,5-dihydro-3H-pyrrolo[3,2-d]pyrimidin-2-yl]sulfanyl}ethyl)propanamide

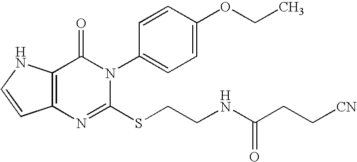

A mixture of 3-(4-ethoxyphenyl)-2-thioxo-1,2,3,5-tetrahydro-4H-pyrrolo[3,2-d]pyrimidin-4-one (144 mg) obtained by the method of Example 1, or a method pursuant to thereto, N-(2-bromoethyl)-3-cyanopropanamide (130 mg) obtained by the method of Reference Example 44, or a method pursuant to thereto, triethylamine (140 μl), sodium iodide (75 mg) and N,N-dimethylformamide (3 ml) was heated to 120° C., and was stirred for 12 hours. The reaction mixture was returned to room temperature, and then was diluted with ethyl acetate (40 ml). This dilution was washed with water and saturated brine, dried over anhydrous magnesium sulfate, and then concentrated under reduced pressure. The resulting residue was purified by chromatography, to obtain a brown solid. This solid was washed with a mixed solvent of 10% ethyl acetate/diethyl ether, and thus the title compound (115 mg) was obtained as a white solid.

¹H NMR (400 MHz, DMSO-d₆) δ ppm 1.37 (3H, t, J=7.0 Hz), 2.40 (2H, t, J=7.0 Hz), 2.61 (2H, t, J=7.0 Hz), 3.12 (2H, t, J=6.6 Hz), 3.34 (2H, td, J=6.6, 5.5 Hz), 4.10 (2H, q, J=7.0 Hz), 6.35 (1H, dd, J=2.4, 1.0 Hz), 7.04 (2H, d, J=8.8 Hz), 7.25 (2H, d, J=8.8 Hz), 7.39 (1H, dd, J=2.6, 2.5 Hz), 8.21 (1H, t, J=5.5 Hz), 12.14 (1H, br. s.).

### Example 62

### N-(2-cyanoethyl)-3-{[3-(4-ethoxyphenyl)-4-oxo-4,5-dihydro-3H-pyrrolo[3,2-d]pyrimidin-2-yl]sulfanyl}propane-1-sulfonamide

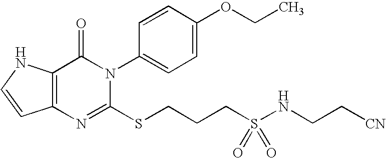

A mixture of 3-(4-ethoxyphenyl)-2-thioxo-1,2,3,5-tetrahydro-4H-pyrrolo[3,2-d]pyrimidin-4-one (144 mg) obtained by the method of Example 1, or a method pursuant to thereto, 3-chloro-N-(2-cyanoethyl)propane-1-sulfonamide (126 mg) obtained by the method of Reference Example 45, or a method pursuant to thereto, triethylamine (140 μl), sodium iodide (75 mg) and N,N-dimethylformamide (3 ml) was heated to 120° C., and was stirred for 24 hours. The reaction mixture was returned to room temperature, and then was concentrated under reduced pressure. To the resulting residue, tetrahydrofuran (20 ml) and water (5 ml) were added, and the mixture was extracted with ethyl acetate. The organic layer was washed with saturated brine, dried over anhydrous magnesium sulfate, and then concentrated under reduced pressure. The resulting residue was purified by chromatography, to obtain a brown solid. This solid was washed with a mixed solvent of 10% ethyl acetate/diethyl ether, and thus the title compound (183 mg) was obtained as a yellowish white solid.

¹H NMR (400 MHz, DMSO-d₆) δ ppm 1.37 (3H, t, J=7.0 Hz), 1.95-2.05 (2H, m), 2.65 (2H, t, J=6.6 Hz), 3.08-3.22 (6H, m), 4.10 (2H, q, J=6.9 Hz), 6.35 (1H, d, J=2.7 Hz), 7.05 (2H, d, J=8.8 Hz), 7.26 (2H, d, J=8.8 Hz), 7.39 (1H, br. s.), 7.52 (1H, br. s.), 12.14 (1H, br. s.).

### Example 63

### N-(cyanomethyl)-4-{[3-(4-ethoxyphenyl)-4-oxo-4,5-dihydro-3H-pyrrolo[3,2-d]pyrimidin-2-yl]sulfanyl}butanamide

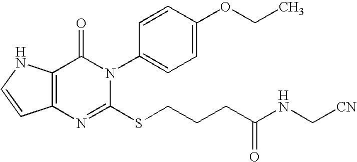

A mixture of 3-(4-ethoxyphenyl)-2-thioxo-1,2,3,5-tetrahydro-4H-pyrrolo[3,2-d]pyrimidin-4-one (144 mg) obtained by the method of Example 1, or a method pursuant to thereto, 4-bromo-N-(cyanomethyl)butanamide (100 mg) obtained by the method of Reference Example 46, or a method pursuant to thereto, triethylamine (140 μl), sodium iodide (75 mg) and N,N-dimethylformamide (3 ml) was heated to 120° C., and was stirred for 24 hours. The reaction mixture was returned to room temperature, and was diluted with ethyl acetate (100 ml). The dilution was washed with water and saturated brine, dried over anhydrous magnesium sulfate, and then concentrated under reduced pressure. The resulting residue was purified by chromatography, and thus the title compound (165 mg) was obtained as a yellowish white solid.

¹H NMR (400 MHz, DMSO-d₆) δ ppm 1.37 (3H, t, J=7.0 Hz), 1.83 (2H, tt, J=7.4, 7.1 Hz), 2.22 (2H, t, J=7.3 Hz), 3.04 (2H, t, J=7.1 Hz), 4.10 (2H, q, J=7.0 Hz), 4.09 (2H, d, J=5.5 Hz), 6.35 (1H, dd, J=2.7, 1.7 Hz), 7.04 (2H, d, J=8.8 Hz), 7.25 (2H, d, J=8.8 Hz), 7.38 (1H, dd, J=2.8, 2.7 Hz), 8.56 (1H, t, J=5.5 Hz), 12.12 (1H, br. s.).

### Example 64

### N-but-3-yn-1-yl-2-{[3-(4-ethoxyphenyl)-4-oxo-4,5-dihydro-3H-pyrrolo[3,2-d]pyrimidin-2-yl]sulfanyl}acetamide

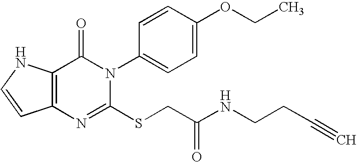

A mixture of 3-(4-ethoxyphenyl)-2-thioxo-1,2,3,5-tetrahydro-4H-pyrrolo[3,2-d]pyrimidin-4-one (144 mg) obtained by the method of Example 1, or a method pursuant to thereto, N-but-3-yn-1-yl-2-chloroacetamide (98 mg) obtained by the method of Reference Example 47, or a method pursuant to thereto, triethylamine (140 μl) and acetonitrile (5 ml) was heated to reflux for 1.5 hours. The reaction mixture was returned to room temperature, and then silica gel was added thereto. The mixture was concentrated under reduced pressure. Subsequently, the resulting residue was purified by chromatography, and thus the title compound (187 mg) was obtained as a white solid.

¹H NMR (400 MHz, DMSO-d₆) δ ppm 1.38 (3H, t, J=7.1 Hz), 2.26 (2H, td, J=7.1, 2.7 Hz), 2.83 (1H, t, J=2.7 Hz), 3.15 (2H, td, J=7.1, 5.9 Hz), 3.78 (2H, s), 4.11 (2H, q, J=7.1 Hz), 6.32 (1H, dd, J=2.6, 1.0 Hz), 7.06 (2H, d, J=8.8 Hz), 7.27 (2H, d, J=8.8 Hz), 7.39 (1H, t, J=2.6 Hz), 8.31 (1H, t, J=5.9 Hz), 12.15 (1H, br. s.).

### Example 65

### N-(2-cyanoethyl)-5-{[3-(4-ethoxyphenyl)-4-oxo-4,5-dihydro-3H-pyrrolo[3,2-d]pyrimidin-2-yl]sulfanyl}pentanamide

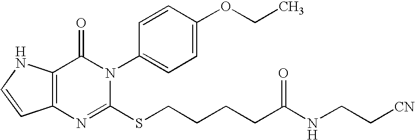

A mixture of 3-(4-ethoxyphenyl)-2-thioxo-1,2,3,5-tetrahydro-4H-pyrrolo[3,2-d]pyrimidin-4-one (144 mg) obtained by the method of Example 1, or a method pursuant to thereto, 5-bromo-N-(2-cyanoethyl)pentanamide (140 mg) obtained by the method of Reference Example 48, or a method pursuant to thereto, triethylamine (140 μl), sodium iodide (75 mg) and N,N-dimethylformamide (3 ml) was heated to 120° C., and was stirred for 24 hours. The reaction mixture was returned to room temperature, and was diluted with ethyl acetate (100 ml). The dilution was washed with water and saturated brine, dried over anhydrous magnesium sulfate, and then concentrated under reduced pressure. The resulting residue was purified by chromatography, and thus the title compound (203 mg) was obtained as a yellowish white solid.

¹H NMR (400 MHz, DMSO-d₆) δ ppm 1.37 (3H, t, J=7.0 Hz), 1.48-1.64 (4H, m), 2.09 (2H, t, J=6.7 Hz), 2.60 (2H, t, J=6.5 Hz), 3.03 (2H, t, J=6.7 Hz), 3.24 (2H, td, J=6.5, 5.6 Hz), 4.10 (2H, q, J=7.0 Hz), 6.35 (1H, dd, J=2.9, 2.0 Hz), 7.04 (2H, d, J=8.8 Hz), 7.23 (2H, d, J=8.8 Hz), 7.38 (1H, t, J=2.9 Hz), 8.19 (1H, t, J=5.6 Hz), 12.11 (1H, t, J=2.0 Hz).

### Example 66

### N-(2-cyanoethyl)-4-{[3-(4-ethoxyphenyl)-4-oxo-4,5-dihydro-3H-pyrrolo[3,2-d]pyrimidin-2-yl]sulfanyl}-N-methylbutanamide

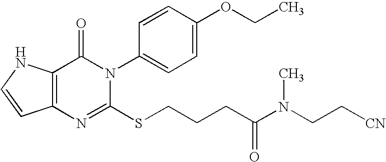

### Example 66a

3-(Methylamino)propanenitrile (841 mg) and triethylamine (2.1 ml) were dissolved in tetrahydrofuran (20 ml), and 4-bromobutanoyl chloride (2.04 g) dissolved in tetrahydrofuran (10 ml) was added thereto dropwise over 5 minutes under ice cooling. The mixture was stirred for 2 hours at room temperature. Subsequently, diethyl ether (20 ml) was added to the reaction solution, and precipitates generated therefrom were filtered, and the filtrate was concentrated under reduced pressure, to obtain a brown crude product. This crude product was purified by chromatography, and thus a brown oily substance (1.13 g) was obtained.

### Example 66b

A mixture of 3-(4-ethoxyphenyl)-2-thioxo-1,2,3,5-tetrahydro-4H-pyrrolo[3,2-d]pyrimidin-4-one (144 mg) obtained by the method of Example 1, or a method pursuant to thereto, the compound (128 mg) obtained in Example (66a), triethylamine (140 μl), sodium iodide (75 mg) and N,N-dimethylformamide (3 ml) was heated to 120° C., and was stirred for 12 hours. The reaction mixture was returned to room temperature, and was diluted with ethyl acetate (100 ml). The dilution was washed with water and saturated brine, dried over anhydrous magnesium sulfate, and then concentrated under reduced pressure. The resulting residue was purified by chromatography, and thus the title compound (112 mg) was obtained as a yellowish white solid.

¹H NMR (400 MHz, DMSO-d₆) δ ppm 1.37 (3H, t, J=7.0 Hz), 1.77-1.86 (2H, m), 2.39 (1.3 H, t, J=7.3 Hz), 2.46 (0.7 H, t, J=7.3 Hz), 2.67 (1.3 H, t, J=6.7 Hz), 2.79 (0.7H, t, J=6.6 Hz), 2.80 (1.1H, s), 2.98 (1.9H, s), 3.05 (0.7H, t, J=7.3 Hz), 3.06 (1.3H, t, J=7.2 Hz), 3.50 (1.3H, t, J=6.7 Hz), 3.57 (0.7H, t, J=6.6 Hz), 4.10 (2H, q, J=7.0 Hz), 6.34 (1H, dd, J=2.7, 2.0 Hz), 7.04 (2H, d, J=8.8 Hz), 7.25 (2H, d, J=8.8 Hz), 7.38 (1H, dd, J=3.0, 2.7 Hz), 12.11 (1H, br. s.).

### Example 67

### N-(2-cyanoethyl)-4-({4-oxo-3-[4-(2,2,2-trifluoroethoxy)phenyl]-4,5-dihydro-3H-pyrrolo[3,2-d]pyrimidin-2-yl sulfanyl)butanamide

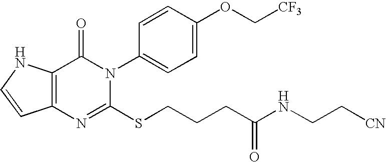

A mixture of 2-thioxo-3-[4-(2,2,2-trifluoroethoxy)phenyl]-1,2,3,5-tetrahydro-4H-pyrrolo[3,2-d]pyrimidin-4-one (171 mg) obtained by the method of Example 2, or a method pursuant to thereto, 4-bromo-N-(2-cyanoethyl)butanamide (131 mg) obtained by the method of Reference Example 49, or a method pursuant to thereto, triethylamine (140 μl), sodium iodide (75 mg) and N,N-dimethylformamide (5 ml) was heated to 100° C., and was stirred for 12 hours. The reaction mixture was returned to room temperature, and then was diluted with ethyl acetate (80 ml). The dilution was washed with water and saturated brine, dried over anhydrous magnesium sulfate, and then concentrated under reduced pressure. The resulting residue was purified by chromatography, and thus the title compound (171 mg) was obtained as a white solid.

¹H NMR (400 MHz, DMSO-d₆) δ ppm 1.83 (2H, tt, J=7.5, 7.0 Hz), 2.17 (2H, t, J=7.5 Hz), 2.61 (2H, t, J=6.4 Hz), 3.06 (2H, t, J=7.1 Hz), 3.24 (2H, td, J=6.4, 5.6 Hz), 4.88 (2H, q, J=8.8 Hz), 6.35 (1H, dd, J=2.7, 1.0 Hz), 7.19 (2H, d, J=8.8 Hz), 7.34 (2H, d, J=8.8 Hz), 7.39 (1H, t, J=2.7 Hz), 8.23 (1H, t, J=5.6 Hz), 12.14 (1H, br. s.).

### Example 68

### Ethyl N-[4-({4-oxo-3-[4-(2,2,2-trifluoroethoxy)phenyl]-4,5-dihydro-3H-pyrrolo[3,2-d]pyrimidin-2-yl}sulfanyl)butanoyl]-β-alaninate

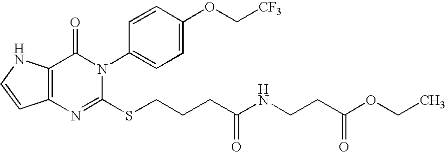

### Example 68a

β-alanine ethyl ester hydrochloride (1.54 g) and triethylamine (3.5 ml) were introduced into acetonitrile (20 ml), and 4-bromobutanoyl chloride (1.86 g) dissolved in acetonitrile (10 ml) was added dropwise thereto over 5 minutes under ice cooling. The mixture was stirred for one hour at room temperature. Subsequently, the reaction solution was diluted with ethyl acetate (100 ml), and precipitates generated therefrom were filtered. The filtrate was washed with water, 1 M hydrochloric acid, a saturated aqueous solution of sodium hydrogen carbonate and saturated brine, dried over anhydrous magnesium sulfate, and then concentrated under reduced pressure, to obtain a yellow crude product. This crude product was purified by chromatography, and thus a colorless oily substance (1.87 g) was obtained.

### Example 68b

To a mixture of 2-thioxo-3-[4-(2,2,2-trifluoroethoxy)phenyl]-1,2,3,5-tetrahydro-4H-pyrrolo[3,2-d]pyrimidin-4-one (102 mg) obtained by the method of Example 2, or a method pursuant to thereto, a 1 M aqueous solution of sodium hydroxide (300 μl) and N,N-dimethylformamide (5 ml), the colorless oily substance (140 mg) obtained in Example (68a) and sodium iodide (75 mg) were added, and the tube was sealed. The mixture was stirred for 15 minutes at 150° C. using a microwave reaction apparatus. The reaction mixture was returned to room temperature, and then was diluted with ethyl acetate (80 ml). The dilution was washed with water and saturated brine, dried over anhydrous magnesium sulfate, and then concentrated under reduced pressure. The resulting residue was purified by chromatography, and thus the title compound (89 mg) was obtained as a white solid.

¹H NMR (400 MHz, DMSO-d₆) δ ppm 1.16 (3H, t, J=7.1 Hz), 1.79 (2H, tt, J=7.2, 7.0 Hz), 2.12 (2H, t, J=7.2 Hz), 2.41 (2H, t, J=6.6 Hz), 3.03 (2H, t, J=7.0 Hz), 3.23 (2H, td, J=6.6, 5.3 Hz), 4.03 (2H, q, J=7.1 Hz), 4.88 (2H, q, J=8.8 Hz), 6.35 (1H, br. s.), 7.19 (2H, d, J=8.9 Hz), 7.33 (2H, d, J=8.9 Hz), 7.39 (1H, br. s.), 7.93 (1H, t, J=5.3 Hz), 12.14 (1H, br. s.)

### Example 69

### N-[4-({4-oxo-3-[4-(2,2,2-trifluoroethoxy)phenyl]-4,5-dihydro-3H-pyrrolo[3,2-d]pyrimidin-2-yl}sulfanyl)butanoyl]-β-alanine

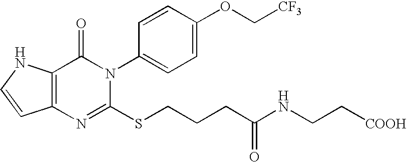

A mixture of ethyl N-[4-({4-oxo-3-[4-(2,2,2-trifluoroethoxy)phenyl]-4,5-dihydro-3H-pyrrolo[3,2-d]pyrimidin-2-yl}sulfanyl)butanoyl]-β-alaninate (87 mg) obtained in Example 68, tetrahydrofuran (1 ml), methanol (2 ml) and a 1 M aqueous solution of sodium hydroxide (2 ml) was stirred for one hour at room temperature. Subsequently, the reaction solution was acidified with 1 M hydrochloric acid, and was extracted with ethyl acetate. The organic layer was washed with saturated brine, dried over anhydrous magnesium sulfate, and then concentrated under reduced pressure, to obtain a colorless crude product. This crude product was purified by chromatography, and thus the title compound (80 mg) was obtained as a white solid.

¹H NMR (400 MHz, DMSO-d₆) δ ppm 1.79 (2H, tt, J=7.5, 7.1 Hz), 2.12 (2H, t, J=7.5 Hz), 2.34 (2H, t, J=6.8 Hz), 3.03 (2H, t, J=7.1 Hz), 3.20 (2H, td, J=6.8, 5.5 Hz), 4.88 (2H, q, J=8.9 Hz), 6.35 (1H, d, J=2.9 Hz), 7.19 (2H, d, J=9.0 Hz), 7.34 (2H, d, J=9.0 Hz), 7.39 (1H, d, J=2.0 Hz), 7.91 (1H, t, J=5.5 Hz), 12.13 (2H, br. s.).

### Example 70

### Tert-butyl 4-({4-oxo-3-[4-(2,2,2-trifluoroethoxy)phenyl]-4,5-dihydro-3H-pyrrolo[3,2-d]pyrimidin-2-yl}sulfanyl)butanoate

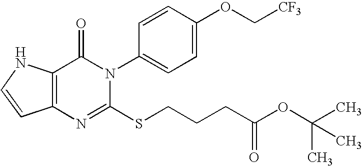

To a mixture of 2-thioxo-3-[4-(2,2,2-trifluoroethoxy)phenyl]-1,2,3,5-tetrahydro-4H-pyrrolo[3,2-d]pyrimidin-4-one (1.0 g) obtained by the method of Example 2, or a method pursuant to thereto, tert-butyl 4-bromobutanoate (714 mg), sodium iodide (436 mg) and N,N-dimethylformamide (30 ml), a 1 M aqueous solution of sodium hydrogen carbonate (3.2 ml) was added. The mixture was stirred for 1.5 hours at 60° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and thus the title compound (1.21 g) was obtained as a white powder.

¹H NMR (300 MHz, CHLOROFORM-d) δ ppm 1.43 (9H, s), 1.85-2.03 (2H, m), 2.32 (2H, t, J=7.4 Hz), 3.16 (2H, t, J=7.2 Hz), 4.42 (2H, q, J=8.0 Hz), 6.41 (1H, t, J=2.5 Hz), 7.08 (2H, d, J=9.1 Hz), 7.19 (1H, t, J=2.8 Hz), 7.25 (2H, d, J=4.5 Hz), 10.17 (1H, br. s.).

### Example 71

### Tert-butyl 4-({5-methyl-4-oxo-3-[4-(2,2,2-trifluoroethoxy)phenyl]-4,5-dihydro-3H-pyrrolo[3,2-d]pyrimidin-2-yl}sulfanyl)butanoate

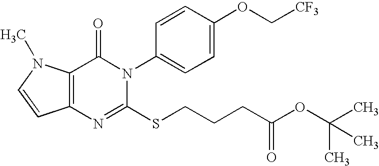

A mixture of tert-butyl 4-({4-oxo-3-[4-(2,2,2-trifluoroethoxy)phenyl]-4,5-dihydro-3H-pyrrolo[3,2-d]pyrimidin-2-yl}sulfanyl)butanoate (232 mg) obtained by the method of Example 70, or a method pursuant to thereto, potassium carbonate (86 mg), iodomethane (500 μl) and N,N-dimethylformamide (5 ml) was stirred for 6 hours at 40° C. The reaction mixture was returned to room temperature, and then was diluted with ethyl acetate (80 ml). The dilution was washed with water and saturated brine, dried over anhydrous magnesium sulfate, and then concentrated under reduced pressure. The resulting residue was purified by chromatography, and thus the title compound (191 mg) was obtained as a white solid.

¹H NMR (300 MHz, DMSO-d₆) δ ppm 1.38 (9H, s), 1.80 (2H, quin, J=7.2 Hz), 2.26 (2H, t, J=7.2 Hz), 3.04 (2H, t, J=7.2 Hz), 3.93 (3H, s), 4.87 (2H, q, J=8.9 Hz), 6.28 (1H, d, J=2.7 Hz), 7.18 (2H, d, J=9.1 Hz), 7.32 (2H, d, J=9.1 Hz), 7.40 (1H, d, J=2.7 Hz).

### Example 72

### N-(2-cyanoethyl)-4-({5-methyl-4-oxo-3-[4-(2,2,2-trifluoroethoxy)phenyl]-4,5-dihydro-3H-pyrrolo[3,2-d]pyrimidin-2-yl}sulfanyl)butanamide

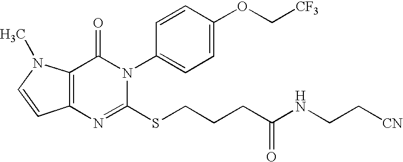

A mixture of tert-butyl 4-({5-methyl-4-oxo-3-[4-(2,2,2-trifluoroethoxy)phenyl]-4,5-dihydro-3H-pyrrolo[3,2-pyrimidin-2-yl}sulfanyl)butanoate (181 mg) obtained in Example 71, 6 M hydrochloric acid (3 ml) and acetonitrile (3 ml) was heated to reflux for 30 minutes. The reaction mixture was returned to room temperature, and was diluted with ethyl acetate (80 ml). The dilution was washed with saturated brine, dried over anhydrous magnesium sulfate, and then concentrated under reduced pressure. To the resulting residue, toluene was added, and the mixture was concentrated under reduced pressure, to obtain a crude product. This crude product was dissolved in N,N-dimethylformamide (4 ml), and 3-aminopropanenitrile (445 mg), 1-ethyl-3-(3-dimethylaminopropyl)carbodiimide hydrochloride (122 mg) and 1-hydroxybenzotriazole (41 mg) were added thereto. The mixture was stirred for 12 hours at room temperature. Subsequently, the reaction mixture was diluted with ethyl acetate (80 ml), and 1 M hydrochloric acid (5 ml) was added thereto. The mixture was washed with water, a saturated aqueous solution of sodium hydrogen carbonate and saturated brine, dried over anhydrous magnesium sulfate, and then concentrated under reduced pressure. The resulting residue was purified by chromatography, and thus the title compound (53 mg) was obtained as a white solid.

¹H NMR (400 MHz, DMSO-d₆) δ ppm 1.81 (2H, tt, J=7.4, 7.1 Hz), 2.16 (2H, t, J=7.4 Hz), 2.60 (2H, t, J=6.5 Hz), 3.04 (2H, t, J=7.1 Hz), 3.24 (2H, td, J=6.5, 5.7 Hz), 3.93 (3H, s), 4.87 (2H, q, J=8.8 Hz), 6.30 (1H, d, J=2.9 Hz), 7.18 (2H, d, J=9.0 Hz), 7.32 (2H, d, J=9.0 Hz), 7.40 (1H, d, J=2.9 Hz), 8.22 (1H, t, J=5.7 Hz).

### Example 73

### 4-({4-Oxo-3-[4-(2,2,2-trifluoroethoxy)phenyl]-4,5-dihydro-3H-pyrrolo[3,2-d]pyrimidin-2-yl)sulfanyl)butanoic acid

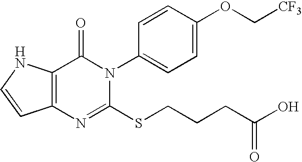

A mixture of tert-butyl 4-({4-oxo-3-[4-(2,2,2-trifluoroethoxy)phenyl]-4,5-dihydro-3H-pyrrolo[3,2-d]pyrimidin-2-yl}sulfanyl)butanoate (1.21 g) obtained by the method of Example 70, or a method pursuant to thereto, 6 M hydrochloric acid (10 ml) and acetonitrile (10 ml) was stirred for 30 minutes at 90° C. The reaction mixture was returned to room temperature, and then acetonitrile was distilled off under reduced pressure. The resulting aqueous solution was extracted with ethyl acetate. The organic layer was washed with saturated brine, dried over anhydrous magnesium sulfate, and then concentrated under reduced pressure. The resulting residue was recrystallized from a mixed solvent of ethyl acetate/hexane, and thus the title compound (850 mg) was obtained as a white powder.

¹H NMR (300 MHz, DMSO-d₆) δ ppm 1.76-1.90 (2H, m), 2.29 (2H, t, J=7.3 Hz), 3.07 (2H, t, J=7.2 Hz), 4.87 (2H, q, J=8.8 Hz), 6.36 (1H, dd, J=2.8, 2.1 Hz), 7.16-7.22 (2H, m), 7.30-7.37 (2H, m), 7.39 (1H, t, J=3.0 Hz), 12.11 (2H, br. s.).

### Example 74

### 2-[(2-Ethoxyethyl)sulfanyl]-3-[4-(2,2,2-trifluoroethoxy)phenyl]-3,5-dihydro-4H-pyrrolo[3,2-d]pyrimidin-4-one

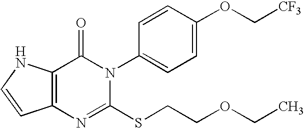

A 1 M aqueous solution of sodium hydrogen carbonate (1.1 ml) was added to a mixture of 2-thioxo-3-[4-(2,2,2-trifluoroethoxy)phenyl]-1,2,3,5-tetrahydro-4H-pyrrolo[3,2-d]pyrimidin-4-one (343 mg) obtained by the method of Example 2, or a method pursuant to thereto, 1-chloro-2-ethoxyethane (109 mg), sodium iodide (149 mg) and N,N-dimethylformamide (10 ml). The mixture was stirred for 2 hours at 100° C. The reaction mixture was returned to room temperature, and then the solvent was distilled off under reduced pressure. The residue was diluted with ethyl acetate, and the dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (168 mg) was obtained as a white powder.

¹H NMR (300 MHz, CHLOROFORM-d) δ ppm 1.19 (3H, t, J=7.0 Hz), 3.34 (2H, t, J=6.5 Hz), 3.52 (2H, q, J=7.0 Hz), 3.68 (2H, t, J=6.5 Hz), 4.41 (2H, q, J=8.0 Hz), 6.42 (1H, d, J=2.3 Hz), 7.04-7.13 (2H, m), 7.22 (1H, t, J=2.8 Hz), 7.23-7.31 (2H, m), 9.73 (1H, br. s.).

### Example 75

### 4-({4-Oxo-3-[4-(2,2,2-trifluoroethoxy)phenyl]-4,5-dihydro-3H-pyrrolo[3,2-d]pyrimidin-2-yl}sulfanyl)-N-(2,2,2-trifluoroethyl)butanamide

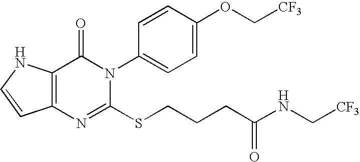

1-Ethyl-3-(3-dimethylaminopropyl)carbodiimide hydrochloride (96 mg) was added to a mixture of 4-({4-oxo-3-[4-(2,2,2-trifluoroethoxy)phenyl]-4,5-dihydro-3H-pyrrolo[3,2-d]pyrimidin-2-yl}sulfanyl)butanoic acid (214 mg) obtained by the method of Example 73, or a method pursuant to thereto, 2,2,2-trifluoroethanamine (50 mg), 1-hydroxybenzotriazole (77 mg) and N,N-dimethylformamide (5 ml), and the resulting mixture was stirred for 15 hours at room temperature. The solvent was distilled off under reduced pressure and then the residue was diluted with ethyl acetate, and the dilution was washed with water and saturated brine, dried over anhydrous magnesium sulfate, and then concentrated under reduced pressure. The resulting residue was recrystallized from a mixed solvent of ethyl acetate/hexane, and thus the title compound (70.3 mg) was obtained as a white powder.

¹H NMR (300 MHz, DMSO-d₆) δ ppm 1.75-1.90 (2H, m), 2.25 (2H, t, J=7.4 Hz), 3.05 (2H, t, J=7.1 Hz), 3.86 (2H, qd, J=9.9, 6.5 Hz), 4.87 (2H, q, J=8.9 Hz), 6.34 (1H, dd, J=2.8, 2.1 Hz), 7.15-7.23 (2H, m), 7.29-7.36 (2H, m), 7.39 (1H, t, J=2.9 Hz), 8.49(1H, t, J=6.4 Hz), 12.12 (1H, br. s.).

### Example 76

### N-cyclopropyl-4-({4-oxo-3-[4-(2,2,2-trifluoroethoxy)phenyl]-4,5-dihydro-3H-pyrrolo[3,2-d]pyrimidin-2-yl}sulfanyl)butanamide

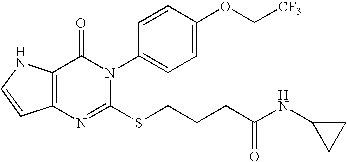

1-Ethyl-3-(3-dimethylaminopropyl)carbodiimide hydrochloride (96 mg) was added to a mixture of 4-({4-oxo-3-[4-(2,2,2-trifluoroethoxy)phenyl]-4,5-dihydro-3H-pyrrolo[3,2-d]pyrimidin-2-yl}sulfanyl)butanoic acid (214 mg) obtained by the method of Example 73, or a method pursuant to thereto, cyclopropylamine (29 mg), 1-hydroxybenzotriazole (77 mg) and N,N-dimethylformamide (5 ml), and the resulting mixture was stirred for 15 hours at room temperature. The solvent was distilled off under reduced pressure, and then the residue was diluted with ethyl acetate. The dilution was washed with water and saturated brine, dried over anhydrous magnesium sulfate, and then concentrated under reduced pressure. The resulting residue was recrystallized from a mixed solvent of ethyl acetate/hexane, and thus the title compound (160 mg) was obtained as a white powder.

¹H NMR (300 MHz, DMSO-d₆) δ ppm 0.28-0.39 (2H, m), 0.51-0.63 (2H, m), 1.74-1.84 (2H, m), 2.08 (2H, t, J=7.3 Hz), 2.57 (1H, td, J=7.3, 3.8 Hz), 3.03 (2H, t, J=7.2 Hz), 4.87 (2H, q, J=8.9 Hz), 6.35 (1H, d, J=2.8 Hz), 7.09-7.25 (2H, m), 7.28-7.36 (2H, m), 7.39 (1H, d, J=2.8 Hz), 7.86(1H, d, J=3.6 Hz), 12.12 (1H, br. s.).

### Example 77

### 2-[(4-Oxo-4-piperidin-1-ylbutyl)sulfanyl]-3-[4-(2,2,2-trifluoroethoxy)phenyl]-3,5-dihydro-4H-pyrrolo[3,2-d]pyrimidin-4-one

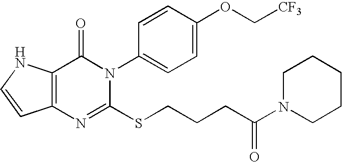

1-Ethyl-3-(3-dimethylaminopropyl)carbodiimide hydrochloride (96 mg) was added to a mixture of 4-({4-oxo-3-[4-(2,2,2-trifluoroethoxy)phenyl]-4,5-dihydro-3H-pyrrolo[3,2-d]pyrimidin-2-yl}sulfanyl)butanoic acid (214 mg) obtained by the method of Example 73, or a method pursuant to thereto, piperidine (43 mg), 1-hydroxybenzotriazole (77 mg) and N,N-dimethylformamide (5 ml), and the resulting mixture was stirred for 15 hours at room temperature. The solvent was distilled off under reduced pressure, and then the residue was diluted with ethyl acetate. The dilution was washed with water and saturated brine, dried over anhydrous magnesium sulfate, and then concentrated under reduced pressure. The resulting residue was recrystallized from a mixed solvent of ethyl acetate/hexane, and thus the title compound (195 mg) was obtained as a white powder.

¹H NMR (300 MHz, DMSO-d₆) δ ppm 1.31-1.49 (4H, m), 1.49-1.62 (2H, m), 1.76-1.85 (2H, m), 2.36 (2H, t, J=7.3 Hz), 3.06 (2H, t, J=7.3 Hz), 3.28-3.43 (4H, m), 4.87 (2H, q, J=8.9 Hz), 6.34 (1H, d, J=2.8 Hz), 7.12-7.21 (2H, m), 7.29-7.36 (2H, m), 7.39 (1H, d, J=2.8 Hz), 12.12 (1H, br. s.).

### Example 78

### 2-{[3-(2-Methoxyethoxy)propyl]sulfanyl}-3-[4-(2,2,2-trifluoroethoxy)phenyl]-3,5-dihydro-4H-pyrrolo[3,2-d]pyrimidin-4-one

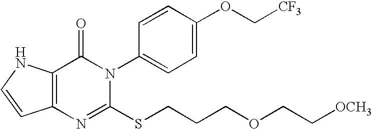

A 1 M aqueous solution of sodium hydrogen carbonate (1.0 ml) was added to a mixture of 2-thioxo-3-[4-(2,2,2-trifluoroethoxy)phenyl]-1,2,3,5-tetrahydro-4H-pyrrolo[3,2-d]pyrimidin-4-one (341 mg) obtained by the method of Example 2, or a method pursuant to thereto, 1-bromo-3-(2-methoxyethoxy)propane (197 mg), sodium iodide (150 mg) and N,N-dimethylformamide (10 ml), and the resulting mixture was stirred for 2 hours at 100° C. The reaction mixture was returned to room temperature, and then the solvent was distilled off under reduced pressure. The residue was diluted with ethyl acetate, and the dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (263 mg) was obtained as a white powder.

¹H NMR (300 MHz, CHLOROFORM-d) δ ppm 1.93-2.05 (2H, m), 3.19 (2H, t, J=7.0 Hz), 3.37 (3H, s), 3.44-3.65 (6H, m), 4.42 (2H, q, J=8.0 Hz), 6.43 (1H, t, J=2.5 Hz), 7.00-7.14 (2H, m), 7.19-7.32 (3H, m), 9.65 (1H, br. s.).

### Example 79

### 2-{[2-(2-Ethoxyethoxy)ethyl]sulfanyl}-3-[4-(2,2,2-trifluoroethoxy)phenyl]-3,5-dihydro-4H-pyrrolo[3,2-d]pyrimidin-4-one

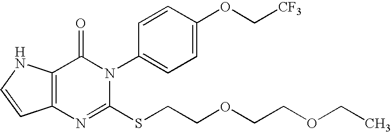

A 1 M aqueous solution of sodium hydrogen carbonate (1.0 ml) was added to a mixture of 2-thioxo-3-[4-(2,2,2-trifluoroethoxy)phenyl]-1,2,3,5 -tetrahydro-4H-pyrrolo[3,2-d]pyrimidin-4-one (341 mg) obtained by the method of Example 2, or a method pursuant to thereto, 1-bromo-2-(2-ethoxyethoxy)ethane (197 mg), sodium iodide (150 mg) and N,N-dimethylformamide (10 ml), and the resulting mixture was stirred for 2 hours at 100° C. The reaction mixture was returned to room temperature, and then the solvent was distilled off under reduced pressure. The residue was diluted with ethyl acetate, and the dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (272 mg) was obtained as a white powder.

¹H NMR (300 MHz, CHLOROFORM-d) δ ppm 1.19 (3H, t, J=7.1 Hz), 3.36 (2H, t, J=6.6 Hz), 3.51 (2H, d, J=7.1 Hz), 3.54-3.60 (2H, m), 3.61-3.67 (2H, m), 3.75 (2H, t, J=6.6 Hz), 4.41 (2H, q, J=8.0 Hz), 6.43 (1H, d, J=2.3 Hz), 7.03-7.12 (2H, m), 7.20-7.30 (3H, m), 9.61 (1H, br. s.).

### Example 80

### 2-{[3-(Methylsulfonyl)propyl]sulfanyl}-3-[4-(2,2,2-trifluoroethoxy)phenyl]-3,5-dihydro-4H-pyrrolo[3,2-d]pyrimidin-4-one

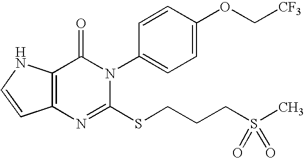

A 1 M aqueous solution of sodium hydrogen carbonate (1.0 ml) was added to a mixture of 2-thioxo-3-[4-(2,2,2-trifluoroethoxy)phenyl]-1,2,3,5-tetrahydro-4H-pyrrolo[3,2-d]pyrimidin-4-one (341 mg) obtained by the method of Example 2, or a method pursuant to thereto, 3-(methylsulfonyl)propyl 4-methylbenzenesulfonate (292 mg) obtained by a method described in a published document, WO 08/1931, or a method pursuant to thereto, sodium iodide (150 mg) and N,N-dimethylformamide (10 ml), and the resulting mixture was stirred for 2 hours at 100° C. The reaction mixture was returned to room temperature, and then the solvent was distilled off under reduced pressure. The residue was diluted with ethyl acetate, and the dilution was washed with water and saturated brine, dried over anhydrous magnesium sulfate, and then concentrated under reduced pressure. The resulting residue was purified by chromatography, and then was recrystallized from ethyl acetate. Thus, the title compound (235 mg) was obtained as a white powder.

¹H NMR (300 MHz, DMSO-d₆) δ ppm 2.00-2.12 (2H, m), 2.98 (3H, s), 3.17 (4H, t, J=7.4 Hz), 4.87 (2H, q, J=8.7 Hz), 6.35 (1H, d, J=3.0 Hz), 7.14-7.25 (2H, m), 7.30-7.44 (3H, m), 12.14 (1H, br. s.).

### Example 81

### 2-[(3-Ethoxypropyl)sulfanyl]-3-[4-(2,2,2-trifluoroethoxy)phenyl]-3,5-dihydro-4H-pyrrolo[3,2-d]pyrimidin-4-one

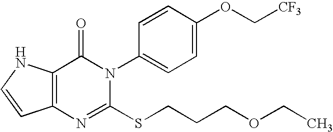

A 1 M aqueous solution of sodium hydrogen carbonate (1.0 ml) was added to a mixture of 2-thioxo-3-[4-(2,2,2-trifluoroethoxy)phenyl]-1,2,3,5-tetrahydro-4H-pyrrolo[3,2-d]pyrimidin-4-one (341 mg) obtained by the method of Example 2, or a method pursuant to thereto, 3-ethoxypropyl 4-methylbenzenesulfonate (258 mg) obtained by a method described in a published document, Canadian Journal of Chemistry (Can. J. Chem.), Vol. 33, p. 1207 (1955) and N,N-dimethylformamide (10 ml), and the resulting mixture was stirred for 2 hours at 100° C. The reaction mixture was returned to room temperature, and then the solvent was distilled off under reduced pressure. The residue was diluted with ethyl acetate, and the dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (199 mg) was obtained as a white powder.

¹H NMR (300 MHz, CHLOROFORM-d) δ ppm 1.17 (3H, t, J=7.0 Hz), 1.89-2.02 (2H, m), 3.20 (2H, t, J=7.0 Hz), 3.37-3.55 (4H, m), 4.42 (2H, q, J=8.0 Hz), 6.43 (1H, d, J=2.3 Hz), 7.04-7.13 (2H, m), 7.21 (1H, t, J=2.8 Hz), 7.23-7.30 (2H, m), 9.91 (1H, br. s.).

### Example 82

### N-(2-cyanoethyl)-4-({3-[4-(cyclobutylmethoxy)phenyl]-4-oxo-4,5-dihydro-3H-pyrrolo[3,2-d]pyrimidin-2-yl}sulfanyl)butanamide

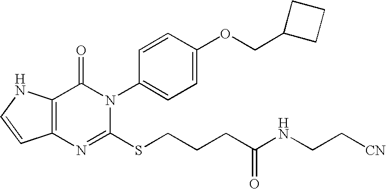

A mixture of 3-[4-(cyclobutylmethoxy)phenyl]-2-thioxo-1,2,3,5-tetrahydro-4H-pyrrolo[3,2-d]pyrimidin-4-one (365 mg) obtained by the method of Example 3, or a method pursuant to thereto, 4-bromo-N-(2-cyanoethyl)butanamide (219 mg) obtained by the method of Reference Example 49, or a method pursuant to thereto, potassium carbonate (221 mg) and N,N-dimethylformamide (5 ml) was stirred for 24 hours at room temperature. Subsequently, water (50 ml) was added to the reaction mixture, and the resulting mixture was extracted with ethyl acetate. The organic layer was washed with saturated brine, dried over anhydrous sodium sulfate, and then concentrated under reduced pressure. The resulting residue was purified by chromatography, and thus the title compound (92 mg) was obtained as a white solid.

¹H NMR (300 MHz, DMSO-d₆) δ ppm 1.74-2.02 (6H, m), 2.03-2.14 (2H, m), 2.17 (2H, t, J=7.4 Hz), 2.60 (2H, t, J=6.4 Hz), 2.68-2.85 (1H, m), 3.05 (2H, t, J=7.1 Hz), 3.24 (2H, td, J=6.4, 5.7 Hz), 4.02 (2H, d, J=6.4 Hz), 6.34 (1H, dd, J=2.9, 1.7 Hz), 7.05 (2H, d, J=8.9 Hz), 7.23 (2H, d, J=8.9 Hz), 7.38 (1H, dd, J=2.9, 2.6 Hz), 8.21 (1H, t, J=5.7 Hz), 12.09 (1H, br. s.).

### Example 83

### Methyl 2-methyl-4-({4-oxo-3-[4-(2,2,2-trifluoroethoxy)phenyl]-4,5-dihydro-3H-pyrrolo[3,2-d]pyrimidin-2-yl}sulfanyl)butanoate

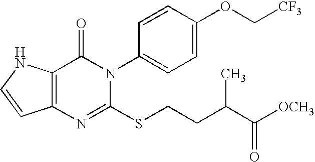

A mixture of 2-thioxo-3-[4-(2,2,2-trifluoroethoxy)phenyl]-1,2,3,5-tetrahydro-4H-pyrrolo[3,2-d]pyrimidin-4-one (341 mg) obtained by the method of Example 2, or a method pursuant to thereto, methyl 3-chloro-2-methylpropanoate (226 mg), N-ethyl-N-(1-methylethyl)propan-2-amine (784 μl) and N,N-dimethylformamide (5 ml) was stirred for 4 hours at 100° C. The reaction mixture was returned to room temperature, and then the solvent was distilled off under reduced pressure. The residue was diluted with ethyl acetate, and the dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (293 mg) was obtained as a white powder.

¹H NMR (300 MHz, CHLOROFORM-d) δ ppm 1.20 (3H, d, J=7.0 Hz), 1.72-1.87 (1H, m), 1.97-2.13 (1H, m), 2.51-2.67 (1H, m), 3.01-3.26 (2H, m), 3.67 (3H, s), 4.42 (2H, q, J=8.0 Hz), 6.43 (1H, d, J=2.9 Hz), 7.08 (2H, d, J=9.0 Hz), 7.21 (1H, t, J=2.9 Hz), 7.26 (2H, d, J=9.0 Hz), 9.90 (1H, br. s.).

### Example 84

### 2-Methyl-4-({4-oxo-3-[4-(2,2,2-trifluoroethoxy)phenyl]-4,5-dihydro-3H-pyrrolo[3,2-d]pyrimidin-2-yl}sulfanyl)butanoic acid

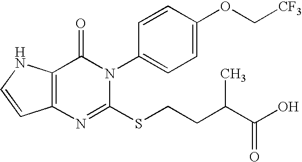

A 1 M aqueous solution of sodium hydroxide (1.8 ml) was added to a mixture of methyl 2-methyl-4-({4-oxo-3-[4-(2,2,2-trifluoroethoxy)phenyl]-4,5-dihydro-3H-pyrrolo[3,2-d]pyrimidin-2-yl)sulfanyl)butanoate (270 mg) obtained in Example 83, tetrahydrofuran (3 ml) and methanol (3 ml), and the mixture was stirred for 2 hours at 50° C. The reaction mixture was returned to room temperature, and then the solvent was distilled off under reduced pressure. The residue was acidified with 1 M hydrochloric acid, and then the mixture was extracted with ethyl acetate. The organic layer was washed with saturated brine, dried over anhydrous magnesium sulfate, and then concentrated under reduced pressure. The resulting residue was recrystallized from ethyl acetate, and thus the title compound (257 mg) was obtained as a white powder.

¹H NMR (300 MHz, DMSO-d₆) δ ppm 1.08 (3H, d, J=6.8 Hz), 1.54-1.74 (1H, m), 1.79-1.96 (1H, m), 2.31-2.48 (1H, m), 3.05 (2H, t, J=7.4 Hz), 4.87 (2H, q, J=8.8 Hz), 6.35 (1H, dd, J=2.5 Hz), 7.19 (2H, d, J=8.9 Hz), 7.33 (2H, d, J=8.9 Hz), 7.38 (1H, t, J=2.5 Hz), 12.12 (1H, br. s.), 12.20 (1H, br. s.).

### Example 85

### N-(2-cyanoethyl)-2-methyl-4-({4-oxo-3-[4-(2,2,2-trifluoroethoxy)phenyl]-4,5-dihydro-3H-pyrrolo[3,2-d]pyrimidin-2-yl)sulfanyl)butanamide

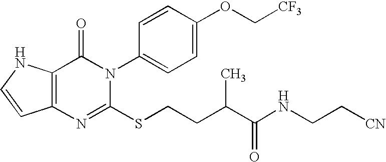

1-Ethyl-3-(3-dimethylaminopropyl)carbodiimide hydrochloride (61 mg) was added to a mixture of 2-methyl-4-({4-oxo-3-[4-(2,2,2-trifluoroethoxy)phenyl]-4,5-dihydro-3H-pyrrolo[3,2-d]pyrimidin-2-yl}sulfanyl)butanoic acid (130 mg) obtained in Example 84, 3-aminopropanenitrile (20 mg), 1-hydroxybenzotriazole (44 mg) and N,N-dimethylformamide (5 ml), and the resulting mixture was stirred for 15 hours at room temperature. The solvent was distilled off under reduced pressure, and then the residue was diluted with ethyl acetate. The dilution was washed with water and saturated brine, dried over anhydrous magnesium sulfate, and then concentrated under reduced pressure. The resulting residue was recrystallized from a mixed solvent of ethyl acetate/hexane, and thus the title compound (140 mg) was obtained as a white powder.

¹H NMR (300 MHz, DMSO-d₆) δ ppm 1.02 (3H, d, J=6.8 Hz), 1.52-1.69 (1H, m), 1.71-1.92 (1H, m), 2.21-2.41 (1H, m), 2.62 (2H, t, J=6.4 Hz), 2.86-3.10 (2H, m), 3.18-3.30 (2H, m), 4.87 (2H, q, J=8.7 Hz), 6.34 (1H, d, J=3.0 Hz), 7.19 (2H, d, J=9.1 Hz), 7.33 (2H, d, J=9.1 Hz), 7.38 (1H, d, J=3.0 Hz), 8.21 (1H, t, J=5.7 Hz), 12.11 (1H, br. s.).

### Example 86

### 4-({4-Oxo-3-[4-(2,2,2-trifluoroethoxy)phenyl]-4,5-dihydro-3H-pyrrolo[3,2-d]pyrimidin-2-yl}sulfanyl)butanenitrile

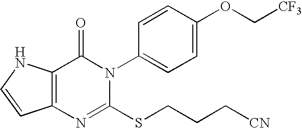

A 1 M aqueous solution of sodium hydrogen carbonate (1.0 ml) was added to a mixture of 2-thioxo-3-[4-(2,2,2-trifluoroethoxy)phenyl]-1,2,3,5-tetrahydro-4H-pyrrolo[3,2-d]pyrimidin-4-one (341 mg) obtained by the method of Example 2, or a method pursuant to thereto, 4-bromobutanenitrile (148 mg), sodium iodide (150 mg) and N,N-dimethylformamide (5 ml), and the resulting mixture was stirred for 3 hours at 80° C. The reaction mixture was returned to room temperature, and then the solvent was distilled off under reduced pressure. The residue was diluted with ethyl acetate, and the dilution was washed with water and saturated brine, dried over anhydrous magnesium sulfate, and then concentrated under reduced pressure. The resulting residue was purified by chromatography, and thus the title compound (300 mg) was obtained as a white powder.

¹H NMR (300 MHz, DMSO-d₆) δ ppm 1.84-2.03 (2H, m), 2.56 (2H, t, J=7.0 Hz), 3.12 (2H, t, J=7.2 Hz), 4.87 (2H, q, J=9.0 Hz), 6.35 (1H, d, J=2.7 Hz), 7.19 (2H, d, J=9.1 Hz), 7.28-7.48 (3H, m), 12.14 (1H, s).

### Example 87

### 5-({4-Oxo-3-[4-(2,2,2-trifluoroethoxy)phenyl]-4,5-dihydro-3H-pyrrolo[3,2-d]pyrimidin-2-yl}sulfanyl)pentanenitrile

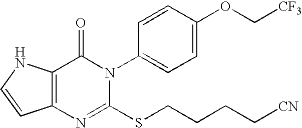

A 1 M aqueous solution of sodium hydrogen carbonate (1.0 ml) was added to a mixture of 2-thioxo-3-[4-(2,2,2-trifluoroethoxy)phenyl]-1,2,3,5-tetrahydro-4H-pyrrolo[3,2-d]pyrimidin-4-one (341 mg) obtained by the method of Example 2, or a method pursuant to thereto, 5-bromopentanenitrile (162 mg), sodium iodide (150 mg) and N,N-dimethylformamide (5 ml), and the resulting mixture was stirred for 3 hours at 80° C. The reaction mixture was returned to room temperature, and then the solvent was distilled off under reduced pressure. The residue was diluted with ethyl acetate, and the dilution was washed with water and saturated brine, dried over anhydrous magnesium sulfate, and then concentrated under reduced pressure. The resulting residue was purified by chromatography, and thus the title compound (258 mg) was obtained as a white powder.

¹H NMR (300 MHz, DMSO-d₆) δ ppm 1.49-1.80 (4H, m), 2.44-2.59 (2H, m), 3.08 (2H, t, J=6.8 Hz), 4.87 (2H, q, J=9.0 Hz), 6.35 (1H, d, J=2.7 Hz), 7.19 (2H, d, J=9.1 Hz), 7.34 (2H, d, J=9.1 Hz), 7.39 (1H, d, J=3.0 Hz), 12.12 (1H, br. s.).

### Example 88

### 6-({4-Oxo-3-[4-(2,2,2-trifluoroethoxy)phenyl]-4,5-dihydro-3H-pyrrolo[3,2-d]pyrimidin-2-yl}sulfanyl)hexanenitrile

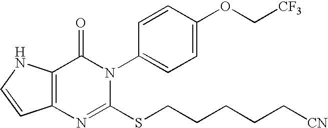

A 1 M aqueous solution of sodium hydrogen carbonate (1.0 ml) was added to a mixture of 2-thioxo-3 -[4-(2,2,2-trifluoroethoxy)phenyl]-1,2,3,5-tetrahydro-4H-pyrrolo[3,2-d]pyrimidin-4-one (341 mg) obtained by the method of Example 2, or a method pursuant to thereto, 6-bromohexanenitrile (176 mg), sodium iodide (150 mg) and N,N-dimethylformamide (5 ml), and the resulting mixture was stirred for 3 hours at 80° C. The reaction mixture was returned to room temperature, and then the solvent was distilled off under reduced pressure. The residue was diluted with ethyl acetate, and the dilution was washed with water and saturated brine, dried over anhydrous magnesium sulfate, and then concentrated under reduced pressure. The resulting residue was purified by chromatography, and was recrystallized from a mixed solvent of ethyl acetate/hexane. Thus, the title compound (306 mg) was obtained as a white powder.

¹H NMR (300 MHz, DMSO-d₆) δ ppm 1.32-1.49 (2H, m), 1.49-1.70 (4H, m), 2.36-2.58 (2H, m), 3.05 (2H, t, J=7.2 Hz), 4.87 (2H, q, J=8.8 Hz), 6.35 (1H, d, J=2.7 Hz), 7.19 (2H, d, J=9.1 Hz), 7.33 (2H, d, J=9.1 Hz), 7.39 (1H, d, J=3.0 Hz), 12.12 (1H, br. s.).

### Example 89

### 2-[2-(2-Ethoxyethoxy)ethoxy]-3-[4-(2,2,2-trifluoroethoxy)phenyl]-3,5-dihydro-4H-pyrrolo[3,2-d]pyrimidin-4-one

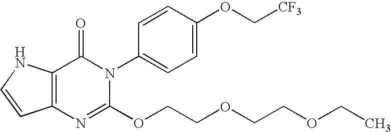

### Example 89a

Phosphoryl chloride (140 μl) was added to a N,N-dimethylformamide solution (10 ml) at room temperature, and the resulting mixture was stirred for 5 minutes at room temperature. Subsequently, 2-thioxo-3-[4-(2,2,2-trifluoroethoxy)phenyl]-1,2,3,5-tetrahydro-4H-pyrrolo[3,2-d]pyrimidin-4-one (341 mg) obtained by the method of Example 2, or a method pursuant to thereto was added thereto, and the resulting mixture was stirred for one hour at 70° C. Phosphoryl chloride (280 μl) was further added thereto, and the resulting mixture was stirred for 2 hours at 70° C. The reaction mixture was returned to room temperature, and then the solvent was distilled off under reduced pressure. The residue was diluted with ethyl acetate, and the dilution was washed with water and saturated brine, dried over anhydrous magnesium sulfate, and then concentrated under reduced pressure. The resulting residue was purified by chromatography, and thus 2-chloro-4-oxo-3-[4-(2,2,2-trifluoroethoxy)phenyl]-3,4-dihydro-5H-pyrrolo[3,2-d]pyrimidine-5-carbaldehyde (185 mg) was obtained as a white powder.

¹H NMR (300 MHz, CHLOROFORM-d) δ ppm 4.43 (2H, q, J=7.9 Hz), 6.71 (1H, dd, J=3.6, 1.1 Hz), 7.08-7.17 (2H, m), 7.21-7.29 (2H, m), 8.03 (1H, d, J=3.6 Hz), 9.91 (1H, s).

### Example 89b

Sodium hydride (60% in oil, 40 mg) was added to a mixture of 2-chloro-4-oxo-3-[4-(2,2,2-trifluoroethoxy)phenyl]-3,4-dihydro-5H-pyrrolo[3,2-d]pyrimidine-5-carbaldehyde (150 mg) obtained in Example 89a), 2-(2-ethoxyethoxy)ethanol (134 mg) and N,N-dimethylformamide (3 ml), and the resulting mixture was stirred for one hour at room temperature. Furthermore, 2-(2-ethoxyethoxy)ethanol (134 mg) and sodium hydride (60% in oil, 40 mg) were sequentially added thereto, and the resulting mixture was stirred for 15 hours at 100° C. The reaction mixture was returned to room temperature, and then the solvent was distilled off under reduced pressure. The residue was diluted with ethyl acetate, and the dilution was washed with water and saturated brine, dried over anhydrous magnesium sulfate, and then concentrated under reduced pressure. The resulting residue was purified by chromatography, and was recrystallized from a mixed solvent of ethyl acetate/hexane. Thus, the title compound (85 mg) was obtained as a white powder.

¹H NMR (300 MHz, CHLOROFORM-d) δ ppm 1.18 (3H, t, J=8.2 Hz), 3.36-3.52 (6H, m), 3.68 (2H, dd, J=5.7, 3.8 Hz), 4.40 (2H, q, J=8.2 Hz), 4.45-4.52 (2H, m), 6.33-6.41 (1H, m), 7.04(2H, d, J=9.1 Hz), 7.17-7.25 (3H, m), 9.51 (1H, br. s.).

### Example 90

### N-(2-cyanoethyl)-4-({4-oxo-3-[4-(3,3,3-trifluoropropoxy)phenyl]-4,5-dihydro-3H-pyrrolo[3,2-d]pyrimidin-2-yl}sulfanyl)butanamide

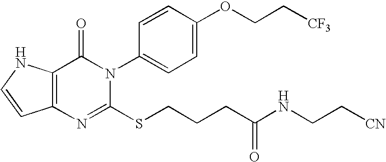

A mixture of 2-thioxo-3-[4-(3,3,3-trifluoropropoxy)phenyl]-1,2,3,5-tetrahydro-4H-pyrrolo[3,2-d]pyrimidin-4-one (432 mg) obtained by the method of Example 4, or a method pursuant to thereto, 4-bromo-N-(2-cyanoethyl)butanamide (350 mg) obtained by the method of Reference Example 49, or a method pursuant to thereto, potassium carbonate (359 mg) and N,N-dimethylformamide (5 ml) was stirred for 24 hours at room temperature. Subsequently, water (50 ml) was added to the reaction mixture, and the resulting mixture was extracted with ethyl acetate. The organic layer was washed with saturated brine, dried over anhydrous sodium sulfate, and then concentrated under reduced pressure. The resulting residue was purified by chromatography, and thus the title compound (253 mg) was obtained as a yellowish white solid.

¹H NMR (300 MHz, DMSO-d₆) δ ppm 1.82 (2H, tt, J=7.4, 7.0 Hz), 2.17 (2H, t, J=7.4 Hz), 2.60 (2H, t, J=6.4 Hz), 2.75-2.94 (2H, m), 3.05 (2H, t, J=7.0 Hz), 3.24 (2H, td, J=6.4, 5.7 Hz), 4.29 (2H, t, J=5.9 Hz), 6.34 (1H, d, J=2.7 Hz), 7.09 (2H, d, J=9.1 Hz), 7.28 (2H, d, J=9.1 Hz), 7.38 (1H, br. s.), 8.20 (1H, t, J=5.7 Hz), 12.10 (1H, s).

### Example 91

### 4-{[3 -(4-Butoxyphenyl)-4-oxo-4,5-dihydro-3H-pyrrolo[3,2-d]pyrimidin-2-yl]sulfanyl}-N-(2-cyanoethyl)butanamide

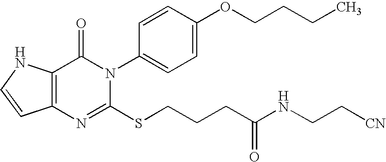

A mixture of 3-(4-butoxyphenyl)-2-thioxo-1,2,3,5-tetrahydro-4H-pyrrolo[3,2-d]pyrimidin-4-one (567 mg) obtained by the method of Example 5, or a method pursuant to thereto, 4-bromo-N-(2-cyanoethyl)butanamide (416 mg) obtained by the method of Reference Example 49, or a method pursuant to thereto, potassium carbonate (442 mg) and N,N-dimethylformamide (5 ml) was stirred for 24 hours at room temperature. Subsequently, water (50 ml) was added to the reaction mixture, and the resulting mixture was extracted with ethyl acetate. The organic layer was washed with saturated brine, dried over anhydrous sodium sulfate, and then concentrated under reduced pressure. The resulting residue was purified by chromatography, and thus the title compound (346 mg) was obtained as a yellowish white solid.

¹H NMR (300 MHz, DMSO-d₆) δ ppm 0.96 (4H, t, J=7.2 Hz), 1.40-1.55 (2H, m), 1.67-1.77(2H, m), 1.82(2H, tt, J=7.4, 7.1 Hz), 2.17 (2H, t, J=7.4 Hz), 2.60 (2H, t, J=6.5 Hz), 3.05 (2H, t, J=7.1 Hz), 3.24 (2H, td, J=6.5, 5.7 Hz), 4.04 (2H, t, J=6.4 Hz), 6.34 (1H, dd, J=3.1, 1.8 Hz), 7.05 (2H, d, J=9.1 Hz), 7.23 (2H, d, J=9.1 Hz), 7.38 (1H, dd, J=3.1, 2.7 Hz), 8.20 (1H, t, J=5.7 Hz), 12.09 (1H, br. s.).

### Example 92

### N-(2-cyanoethyl)-4-({3-[4-(cyclopropylmethoxy)phenyl]-4-oxo-4,5-dihydro-3H-pyrrolo[3,2-d]pyrimidin-2-yl}sulfanyl)butanamide

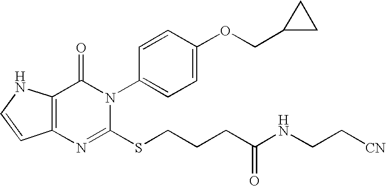

A mixture of 3-[4-(cyclopropylmethoxy)phenyl]-2-thioxo-1,2,3,5-tetrahydro-4H-pyrrolo[3,2-d]pyrimidin-4-one (425 mg) obtained by the method of Example 6, or a method pursuant to thereto, 4-bromo-N-(2-cyanoethyl)butanamide (307 mg) obtained by the method of Reference Example 49, or a method pursuant to thereto, potassium carbonate (359 mg), and N,N-dimethylformamide (5 ml) was stirred for 24 hours at room temperature. Subsequently, water (50 ml) was added to the reaction mixture, and the resulting mixture was extracted with ethyl acetate. The organic layer was washed with saturated brine, dried over anhydrous sodium sulfate, and then concentrated under reduced pressure. The resulting residue was purified by chromatography, and thus the title compound (253 mg) was obtained as a yellowish white solid.

¹H NMR (300 MHz, DMSO-d₆) δ ppm 0.30-0.44 (2H, m), 0.54-0.68 (2H, m), 1.15-1.39 (1H, m), 1.82 (2H, tt, J=7.4, 7.0 Hz), 2.17 (2H, t, J=7.4 Hz), 2.60 (2H, t, J=6.4 Hz), 3.04 (2H, t, J=7.0 Hz), 3.24 (2H, td, J=6.4, 5.7 Hz), 3.89 (2H, d, J=6.8 Hz), 6.34 (1H, d, J=2.7 Hz), 7.04 (2H, d, J=8.9 Hz), 7.23 (2H, d, J=8.9 Hz), 7.37 (1H, d, J=2.7 Hz), 8.20 (1H, t, J=5.7 Hz), 12.09 (1H, s).

### Example 93

### N-(2-cyanoethyl)-4-[(3-{4-[(1-methylcyclopropyl)methoxy]phenyl}-4-oxo-4,5-dihydro-3H-pyrrolo[3,2-d]pyrimidin-2-yl)sulfanyl]butanamide

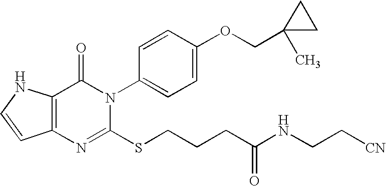

A mixture of 3-4-[(1-methylcyclopropyl)methoxy]phenyl}-2-thioxo-1,2,3,5-tetrahydro-4H-pyrrolo[3,2-d]pyrimidin-4-one (517 mg) obtained by the method of Example 7, or a method pursuant to thereto, 4-bromo-N-(2-cyanoethyl)butanamide (416 mg) obtained by the method of Reference Example 49, or a method pursuant to thereto, potassium carbonate (441 mg) and N,N-dimethylformamide (5 ml) was stirred for 24 hours at room temperature. Subsequently, water (50 ml) was added to the reaction mixture, and the resulting mixture was extracted with ethyl acetate. The organic layer was washed with saturated brine, dried over anhydrous sodium sulfate, and then concentrated under reduced pressure. The resulting residue was purified by chromatography, and thus the title compound (220 mg) was obtained as a yellowish white solid.

¹H NMR (300 MHz, DMSO-d₆) δ ppm 0.36-0.49 (2H, m), 0.51-0.63 (2H, m), 1.21 (3H, s), 1.82 (2H, tt, J=7.4, 7.2 Hz), 2.17 (2H, t, J=7.4 Hz), 2.60 (2H, t, J→6.4 Hz), 3.05 (2H, t, J=7.2 Hz), 3.24 (2H, td, J=6.4, 5.7 Hz), 3.82 (2H, s), 6.34 (1H, d, J=2.5 Hz), 7.04 (2H, d, J=8.9 Hz), 7.23 (2H, d, J=8.9 Hz), 7.37 (1H, t, J=2.5 Hz), 8.20 (1H, t, J=5.7 Hz), 12.09 (1H, br. s.).

### Example 94

### N-(2-cyanoethyl)-4-({3-[4-(3,3-dimethylbutoxy)phenyl]-4-oxo-4,5-dihydro-3H-pyrrolo[3,2-d]pyrimidin-2-yl}sulfanyl)butanamide

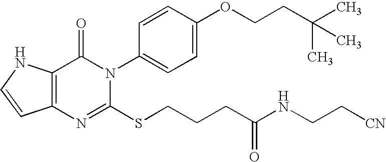

A mixture of 3-[4-(3,3-dimethylbutoxy)phenyl]-2-thioxo-1,2,3,5-tetrahydro-4H-pyrrolo[3,2-d]pyrimidin-4-one (427 mg) obtained by the method of Example 8, or a method pursuant to thereto, 4-bromo-N-(2-cyanoethyl)butanamide (285 mg) obtained by the method of Reference Example 49, or a method pursuant to thereto, potassium carbonate (248 mg) and N,N-dimethylformamide (5 ml) was stirred for 24 hours at room temperature. Subsequently, water (50 ml) was added to the reaction mixture, and the resulting mixture was extracted with ethyl acetate. The organic layer was washed with saturated brine, dried over anhydrous sodium sulfate, and then concentrated under reduced pressure. The resulting residue was purified by chromatography, and then was recrystallized from a mixed solvent of ethyl acetate/hexane. Thus, the title compound (271 mg) was obtained as a white solid.

¹H NMR (300 MHz, DMSO-d₆) δ ppm 0.99 (9H, s), 1.71 (2H, t, J=7.2 Hz), 1.82 (2H, tt, J=7.4, 7.2 Hz), 2.17 (2H, t, J=7.4 Hz), 2.60 (2H, J=6.4 Hz), 3.05 (2H, t, J=7.2 Hz), 3.24 (2H, td, J=6.4, 5.7 Hz), 4.10 (2H, t, J=7.2 Hz), 6.34 (1H, d, J=3.0 Hz), 7.06 (2H, d, J=9.0 Hz), 7.24 (2H, d, J=9.0 Hz), 7.38 (1H, d, J=3.0 Hz), 8.20 (1H, t, J=5.7 Hz), 12.09 (1H, s).

### Example 95

### N-(2-cyanoethyl)-4-[(3-{4-[(2,2-difluorocyclopropyl)methoxy]phenyl}-4-oxo-4,5-dihydro-3H-pyrrolo[3,2-d]pyrimidin-2-yl)sulfanyl]butanamide

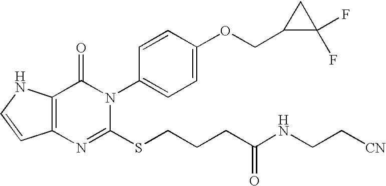

A mixture of 3-{4-[(2,2-difluorocyclopropyl)methoxy]phenyl}-2-thioxo-1,2,3,5-tetrahydro-4H-pyrrolo[3,2-d]pyrimidin-4-one (409 mg) obtained by the method of Example 9, or a method pursuant to thereto, 4-bromo-N-(2-cyanoethyl)butanamide (442 mg) obtained by the method of Reference Example 49, or a method pursuant to thereto, potassium carbonate (331 mg) and N,N-dimethylformamide (5 ml) was stirred for 24 hours at room temperature. Subsequently, water (50 ml) was added to the reaction mixture, and the resulting mixture was extracted with ethyl acetate. The organic layer was washed with saturated brine, dried over anhydrous sodium sulfate, and then concentrated under reduced pressure. The resulting residue was purified by chromatography; and then was recrystallized from a mixed solvent of ethyl acetate/hexane. Thus, the title compound (108 mg) was obtained as a yellowish white solid.

¹H NMR (300 MHz, DMSO-d₆) δ ppm 1.45-1.61 (1H, m), 1.68-1.93 (3H, m), 2.17 (2H, t, J=7.4 Hz), 2.22-2.42 (1H, m), 2.60 (2H, t, J=6.4 Hz), 3.05 (2H, t, J=7.2 Hz), 3.24 (2H, td, J=6.4, 5.7 Hz), 3.84-4.42 (2H, m), 6.34 (1H, d, J=2.6 Hz), 7.09 (2H, d, J=9.0 Hz), 7.27 (2H, d, J=9.0 Hz), 7.38 (1H, d, J=2.6 Hz), 8.20 (1H, t, J=5.7 Hz), 12.10 (1H, s).

### Example 96

### N-(2-cyanoethyl)-4-[(3-{4-[(2-methylcyclopropyl)methoxy]phenyl}-4-oxo-4,5-dihydro-3H-pyrrolo[3,2-d]pyrimidin-2-yl)sulfanyl]butanamide

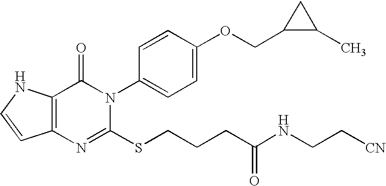

A mixture of 3-{4-[(2-methylcyclopropyl)methoxy]phenyl}-2-thioxo-1,2,3,5-tetrahydro-4H-pyrrolo[3,2-d]pyrimidin-4-one (536 mg) obtained by the method of Example 10, or a method pursuant to thereto, 4-bromo-N-(2-cyanoethyl)butanamide (394 mg) obtained by the method of Reference Example 49, or a method pursuant to thereto, potassium carbonate (441 mg) and N,N-dimethylformamide (5 ml) was stirred for 24 hours at room temperature. Subsequently, water (50 ml) was added to the reaction mixture, and the resulting mixture was extracted with ethyl acetate. The organic layer was washed with saturated brine, dried over anhydrous sodium sulfate, and then concentrated under reduced pressure. The resulting residue was purified by chromatography, and then was recrystallized from a mixed solvent of ethyl acetate/hexane. Thus, the title compound (251 mg) was obtained as a white solid.

¹H NMR (300 MHz, DMSO-d₆) δ ppm 0.28-0.43 (1H, m), 0.47-0.57 (1H, m), 0.70-0.87 (1H, m), 0.92-1.03 (1H, m), 1.07 (3H, d, J=6.0 Hz), 1.82 (2H, tt, j=7.4, 7.1 Hz), 2.17 (2H, t, J=7.4 Hz), 2.60 (2H, t, J=6.4 Hz), 3.04 (2H, t, J=7.1 Hz), 3.24 (2H, td, J=6.4, 5.7 Hz), 3.77-3.99 (2H, m), 6.34 (1H, d, J=2.6 Hz), 7.03 (2H, d, J=9.0 Hz), 7.22 (2H, d, J=9.0 Hz), 7.37 (1H, d, J=2.6 Hz), 8.20 (1H, t, J=5.7 Hz), 12.09 (1H, s).

### Example 97

### 2-{([4-(3 -Hydroxypyrrolidin-1-yl)-4-oxobutyl]sulfanyl}-3-[4-(2,2,2-trifluoroethoxy)phenyl]-3,5-dihydro-4H-pyrrolo[3,2-d]pyrimidin-4-one

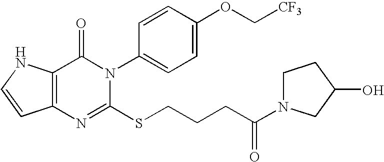

1-Ethyl-3-(3-dimethylaminopropyl)carbodiimide hydrochloride (96 mg) was added to a mixture of 4-({4-oxo-3-[4-(2,2,2-trifluoroethoxy)phenyl]-4,5-dihydro-3H-pyrrolo[3,2-d]pyrimidin-2-yl}sulfanyl)butanoic acid (214 mg) obtained by the method of Example 73, or a method pursuant to thereto, pyrrolidin-3-ol (44 mg), 1-hydroxybenzotriazole (77 mg) and N,N-dimethylformamide (5 ml), and the resulting mixture was stirred for 3 days at room temperature. The solvent was distilled off under reduced pressure, and then the residue was diluted with ethyl acetate. The dilution was washed with water, a saturated aqueous solution of sodium hydrogen carbonate and saturated brine, dried over anhydrous magnesium sulfate, and then concentrated under reduced pressure. The resulting residue was recrystallized from a mixed solvent of ethyl acetate/hexane, and thus the title compound (168 mg) was obtained as a white powder.

¹H NMR (300 MHz, DMSO-d₆) δ ppm 1.61-1.94 (4H, m), 2.19-2.37 (2H, m), 3.07(2H, t, J=7.2 Hz), 3.14-3.51 (4H, m), 4.24 (1H, m), 4.74-5.01 (3H, m), 6.35 (1H, d, J=2.7 Hz), 7.19 (2H, d, J=9.1 Hz), 7.34 (2H, d, J=9.1 Hz), 7.38 (1H, d, J=3.0 Hz), 12.11 (1H, s).

### Example 98

### 2-{[4-(4-Hydroxypiperidin-1-yl)-4-oxobutyl]sulfanyl}-3-[4-(2,2,2-trifluoroethoxy)phenyl]-3,5-dihydro-4H-pyrrolo[3,2-d]pyrimidin-4-one

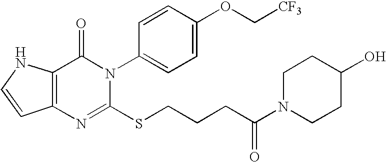

1-Ethyl-3-(3-dimethylaminopropyl)carbodiimide hydrochloride (96 mg) was added to a mixture of 4-({4-oxo-3-[4-(2,2,2-trifluoroethoxy)phenyl]-4,5-dihydro-3H-pyrrolo[3,2-d]pyrimidin-2-yl}sulfanyl)butanoic acid (214 mg) obtained by the method of Example 73, or a method pursuant to thereto, piperidin-4-ol (52 mg), 1-hydroxybenzotriazole (77 mg) and N,N-dimethylformamide (5 ml), and the resulting mixture was stirred for 3 days at room temperature. The solvent was distilled off under reduced pressure, and then the residue was diluted with ethyl acetate. The dilution was washed with water, a saturated aqueous solution of sodium hydrogen carbonate and saturated brine, dried over anhydrous magnesium sulfate, and then concentrated under reduced pressure. The resulting residue was recrystallized from a mixed solvent of ethyl acetate/hexane, and thus the title compound (180 mg) was obtained as a white powder.

¹H NMR (300 MHz, DMSO-d₆) δ ppm 1.10-1.40 (2H, m), 1.56-1.90 (4H, m), 2.38 (2H, t, J=7.2 Hz), 2.86-3.17 (4H, m), 3.53-3.74 (2H, m), 3.79-3.93 (1H, m), 4.71 (1H, d, J=4.2 Hz), 4.87 (2H, q, J=9.1 Hz), 6.34 (1H, d, J=3.0 Hz), 7.19 (2H, d, J=9.1 Hz), 7.33 (2H, d, J=9.1 Hz), 7.38 (1H, d, J=3.0 Hz), 12.12 (1H, s).

### Example 99

### N-(2-hydroxy-2-methylpropyl)-4-({4-oxo-3-[4-(2,2,2-trifluoroethoxy)phenyl]-4,5-dihydro-3H-pyrrolo[3,2-d]pyrimidin-2-yl}sulfanyl)butanamide

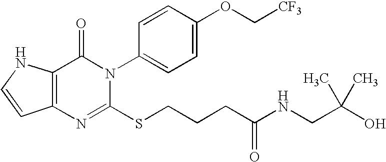

1-Ethyl-3-(3-dimethylaminopropyl)carbodiimide hydrochloride (96 mg) was added to a mixture of 4-({4-oxo-3-[4-(2,2,2-trifluoroethoxy)phenyl]-4,5-dihydro-3H-pyrrolo[3,2-d]pyrimidin-2-yl}sulfanyObutanoic acid (214 mg) obtained by the method of Example 73, or a method pursuant to thereto, 1-amino-2-methylpropan-2-ol (45 mg), 1-hydroxybenzotriazole (77 mg) and N,N-dimethylformamide (5 ml), and the resulting mixture was stirred for 3 days at room temperature. The solvent was distilled off under reduced pressure, and then the residue was diluted with ethyl acetate. The dilution was washed with water, a saturated aqueous solution of sodium hydrogen carbonate and saturated brine, dried over anhydrous magnesium sulfate, and then concentrated under reduced pressure. The resulting residue was recrystallized from a mixed solvent of ethyl acetate/hexane, and thus the title compound (165 mg) was obtained as a white powder.

¹H NMR (300 MHz, DMSO-d₆) δ ppm 1.02 (6H, s), 1.75-1.88 (2H, m), 2.20 (2H, t, J=7.2 Hz), 2.99 (2H, d, J=6.1 Hz), 3.05 (2H, t, J=7.2 Hz), 4.40 (1H, s), 4.87 (2H, q, J=9.0 Hz), 6.34 (1H, d, J=2.7 Hz), 7.19 (2H, d, J=8.7 Hz), 7.34 (2H, d, J=8.7 Hz), 7.38 (1H, d, J=3.0 Hz), 7.69 (1H, t, J=6.1 Hz), 12.11 (1H, s).

### Example 100

### N-(2-hydroxy-1,1-dimethylethyl)-4-({4-oxo-3-[4-(2,2,2-trifluoroethoxy)phenyl]-4,5-dihydro-3H-pyrrolo[3,2-d]pyrimidin-2-yl}sulfanyl)butanamide

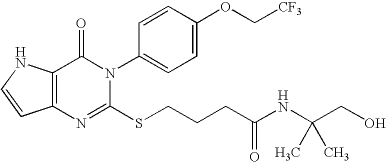

1-Ethyl-3-(3-dimethylaminopropyl)carbodiimide hydrochloride (96 mg) was added to a mixture of 4-({4-oxo-3-[4-(2,2,2-trifluoroethoxy)phenyl]-4,5-dihydro-3H-pyrrolo[3,2-d]pyrimidin-2-yl}sulfanyl)butanoic acid (214 mg) obtained by the method of Example 73, or a method pursuant to thereto, 2-amino-2-methylpropan-1-ol (45 mg), 1-hydroxybenzotriazole (77 mg) and N,N-dimethylformamide (5 ml), and the resulting mixture was stirred for 3 days at room temperature. The solvent was distilled off under reduced pressure, and then the residue was diluted with ethyl acetate. The dilution was washed with water, a saturated aqueous solution of sodium hydrogen carbonate and saturated brine, dried over anhydrous magnesium sulfate, and then concentrated under reduced pressure. The resulting residue was recrystallized from a mixed solvent of ethyl acetate/hexane, and thus the title compound (83 mg) was obtained as a white powder.

¹H NMR (300 MHz, DMSO-d₆) δ ppm 1.14 (6H, s), 1.78 (2H, m), 2.12 (2H, t, J=7.3 Hz), 3.04 (2H, t, J=7.3 Hz), 3.35 (2H, d, J=5.8 Hz), 4.72-4.97 (3H, m), 6.35 (1H, d, J=2.8 Hz), 7.14-7.23 (2H, m), 7.27 (1H, s), 7.29-7.37 (2H, m), 7.39 (1H, d, J=2.8 Hz), 12.12 (1H, s).

### Example 101

### 2-[3-(1H-tetrazol-5-yl)propyl]sulfanyl}-3-[4-(2,2,2-trifluoroethoxy)phenyl]-3,5-dihydro-4H-pyrrolo[3,2-d]pyrimidin-4-one

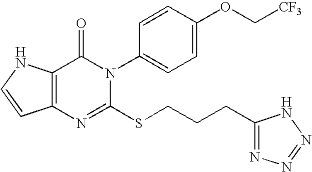

A mixture of 4-({4-oxo-3-[4-(2,2,2-trifluoroethoxy)phenyl]-4,5-dihydro-3H-pyrrolo[3,2-d]pyrimidin-2-yl}sulfanyl)butanenitrile (150 mg) obtained in Example 86, azido(trimethyl)silane (212 mg), dibutyl(oxo)stannum (10 mg) and toluene (5 ml) was stirred for 4 days at 120° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by reverse phase chromatography, and then was recrystallized from ethyl acetate. Thus, the title compound (88 mg) was obtained as a white powder.

¹H NMR (300 MHz, DMSO-d₆) δ ppm 1.99-2.14 (2H, m), 2.95 (2H, t, J=7.5 Hz), 3.12 (2H, t, J=7.1 Hz), 4.87 (2H, q, J=8.9 Hz), 6.31 (1H, dd, J=2.6, 2.1 Hz), 7.14-7.24 (2H, m), 7.30-7.37 (2H, m), 7.39 (1H, t, J=2.6 Hz), 12.13 (1H, br. s.).

### Example 102

### 2-[(1-Methylethyl)sulfanyl]-3-[4-(2,2,2-trifluoroethoxy)phenyl]-3,5-dihydro-4H-pyrrolo[3,2-d]pyrimidin-4-one

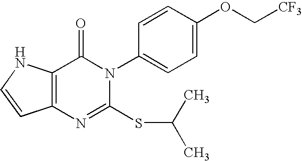

A 1 M aqueous solution of sodium hydrogen carbonate (0.3 ml) was added to a mixture of 2-thioxo-3-[4-(2,2,2-trifluoroethoxy)phenyl]-1,2,3,5-tetrahydro-4H-pyrrolo[3,2-d]pyrimidin-4-one (100 mg) obtained by the method of Example 2, or a method pursuant to thereto, 2-iodopropane (29 μl) and N,N-dimethylformamide (3 ml), and the resulting mixture was stirred for one hour at 80° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (76 mg) was obtained as a white powder.

¹H NMR (300 MHz, CHLOROFORM-d) δ ppm 1.35 (6H, d, J=6.8 Hz), 3.84-4.05 (1H, m), 4.41 (2H, q, J=8.2 Hz), 6.44 (1H, t, J=2.5 Hz), 7.08 (2H, d, J=8.7 Hz), 7.18-7.25 (3H, m), 9.54 (1H, br. s.).

### Example 103

### 3-[4-(2,2,2-Trifluoroethoxy)phenyl]-2-[(2,2,2-trifluoroethyl)sulfanyl]-3,5-dihydro-4H-pyrrolo[3,2-d]pyrimidin-4-one

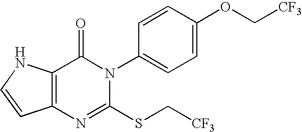

A 1 M aqueous solution of sodium hydrogen carbonate (0.3 ml) was added to a mixture of 2-thioxo-3-[4-(2,2,2-trifluoroethoxy)phenyl]-1,2,3,5-tetrahydro-4H-pyrrolo[3,2-d]pyrimidin-4-one (100 mg) obtained by the method of Example 2, or a method pursuant to thereto, 1,1,1-trifluoro-2-iodoethane (290 and N,N-dimethylformamide (3 ml), and the resulting mixture was stirred for one hour at 80° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (63 mg) was obtained as a white powder.

¹H NMR (300 MHz, CHLOROFORM-d) δ ppm 4.00 (2H, q, J=9.8 Hz), 4.43 (2H, q, J=8.2 Hz), 6.31-6.58 (1H, m), 7.07-7.14 (2H, m), 7.22-7.29 (2H, m), 7.29-7.32 (1H, m), 9.69 (1H, br. s.).

### Example 104

### 2-(Ethylsulfanyl)-3-[4-(2,2,2-trifluoroethoxy)phenyl]-3,5-dihydro-4H-pyrrolo[3,2-d]pyrimidin-4-one

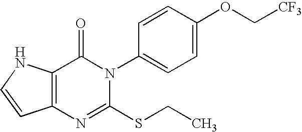

A 1 M aqueous solution of sodium hydrogen carbonate (0.3 ml) was added to a mixture of 2-thioxo-3-[4-(2,2,2-trifluoroethoxy)phenyl]-1,2,3,5-tetrahydro-4H-pyrrolo[3,2-d]pyrimidin-4-one (100 mg) obtained by the method of Example 2, or a method pursuant to thereto, iodoethane (23 μl) and N,N-dimethylformamide (3 ml), and the resulting mixture was stirred for one hour at 80° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (68 mg) was obtained as a white powder.

¹H NMR (300 MHz, CHLOROFORM-d) δ ppm 1.32 (3H, t, J=7.3 Hz), 3.12 (2H, q, J=7.3 Hz), 4.42 (2H, q, J=8.2 Hz), 6.39-6.49 (1H, m), 7.02-7.14 (2H, m), 7.17-7.23 (1H, m), 7.23-7.31 (2H, m), 9.84 (1H, br. s.).

### Example 105

### 2-(Propylsulfanyl)-3-[4-(2,2,2-trifluoroethoxy)phenyl]-3,5-dihydro-4H-pyrrolo[3,2-d]pyrimidin-4-one

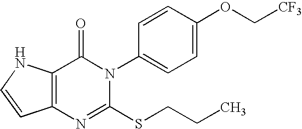

A 1 M aqueous solution of sodium hydrogen carbonate (0.3 ml) was added to a mixture of 2-thioxo-3-[4-(2,2,2-trifluoroethoxy)phenyl]-1,2,3,5-tetrahydro-4H-pyrrolo[3,2-d]pyrimidin-4-one (100 mg) obtained by the method of Example 2, or a method pursuant to thereto, 1-iodopropane (29 μl) and N,N-dimethylformamide (3 ml), and the resulting mixture was stirred for one hour at 80° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (88 mg) was obtained as a white powder.

¹H NMR (300 MHz, CHLOROFORM-d) δ ppm 0.99 (3H, t, J=7.4 Hz), 1.55-1.78 (2H, m), 3.09 (1H, t, J=7.2 Hz), 4.42 (2H, q, J=8.1 Hz), 6.39-6.49 (1H, m), 7.03-7.14 (2H, m), 7.18-7.23 (1H, m), 7.23-7.32(3H, m), 9.78 (1H, br. s.).

### Example 106

### 2-{[3-(Morpholin-4-ylsulfonyl)propyl]sulfanyl}-3-[4-(2,2,2-trifluoroethoxy)phenyl]-3,5-dihydro-4H-pyrrolo[3,2-d]pyrimidin-4-one

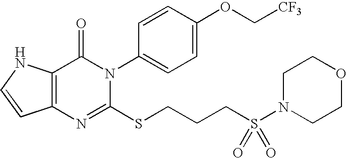

A 1 M aqueous solution of sodium hydrogen carbonate (0.59 ml) was added to a mixture of 2-thioxo-3-[4-(2,2,2-trifluoroethoxy)phenyl]-1,2,3,5-tetrahydro-4H-pyrrolo[3,2-d]pyrimidin-4-one (200 mg) obtained by the method of Example 2, or a method pursuant to thereto, 4-[(3-chloropropyl)sulfonyl]morpholine (133 mg) obtained by a method described in a published document, Journal of Organic Chemistry (J. Org. Chem.), Vol. 34, p. 3324 (1969), or a method pursuant to thereto, and N,N-dimethylformamide (10 ml), and the resulting mixture was stirred for 2 hours at 100° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (107 mg) was obtained as a white powder.

¹H NMR (300 MHz, DMSO-d₆) δ ppm 1.98-2.10 (2H, m), 3.10-3.19 (8H m), 3.59-3.64 (4H, m), 4.87 (2H, q, J=9.1 Hz), 6.35 (1H, d, J=2.7 Hz), 7.19 (2H, d, J=9.1 Hz), 7.36 (2H, d, J=8.7 Hz), 7.40 (1H, d, J=3.0 Hz), 12.15 (1H, s).

### Example 107

### 3-({4-Oxo-3-[4-(2,2,2-trifluoroethoxy)phenyl]-4,5-dihydro-3H-pyrrolo[3,2-d]pyrimidin-2-yl}sulfanyl)-N-(2,2,2-trifluoroethyl)propane-1-sulfonamide

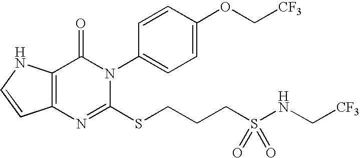

Triethylamine (164 μl) was added to a mixture of 2-thioxo-3-[4-(2,2,2-trifluoroethoxy)phenyl]-1,2,3,5-tetrahydro-4H-pyrrolo[3,2-d]pyrimidin-4-one (200 mg) obtained by the method of Example 2, or a method pursuant to thereto, 3-chloro-N-(2,2,2-trifluoroethyl)propane-1-sulfonamide (141 mg) obtained by a method described in a published document, Journal of Organic Chemistry (J. Org. Chem.), Vol. 34, p. 3324 (1969), or a method pursuant to thereto, sodium iodide (88 mg) and N,N-dimethylformamide (5 ml), and the resulting mixture was stirred for 2 hours at 120° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (69 mg) was obtained as a white powder.

¹H NMR (300 MHz, DMSO-d₆) δ ppm 1.95-2.10 (2H, m), 3.09-3.22 (4H, m), 3.76 (2H, q, J=9.6 Hz), 4.87 (2H, q, J=8.9 Hz), 6.35 (1H, d, J=1.7 Hz), 7.16-7.23 (2H, m), 7.33-7.37 (2H, m), 7.40 (1H, t, J=2.5 Hz), 8.08 (1H, br. s.), 12.15 (1H, br. s.).

### Example 108

### N-cyclopropyl-3-({4-oxo-3-[4-(2,2,2-trifluoroethoxy)phenyl]-4,5-dihydro-3H-pyrrolo[3,2-d]pyrimidin-2-yl}sulfanyl)propane-1-sulfonamide

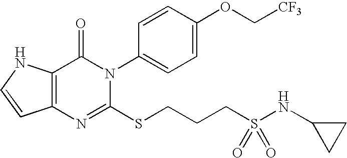

Triethylamine (164 μl) was added to a mixture of 2-thioxo-3-[4-(2,2,2-trifluoroethoxy)phenyl]-1,2,3,5-tetrahydro-4H-pyrrolo[3,2-d]pyrimidin-4-one (200 mg) obtained by the method of Example 2, or a method pursuant to thereto, 3-chloro-N-cyclopropylpropane-1-sulfonamide (117 mg) obtained by a method described in a published document, Journal of Organic Chemistry (J. Org. Chem.), Vol. 34, p. 3324 (1969), or a method pursuant to thereto, sodium iodide (88 mg) and N,N-dimethylformamide (5 ml), and the resulting mixture was stirred for 2 hours at 100° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (198 mg) was obtained as a white powder.

¹H NMR (300 MHz, DMSO-d₆) δ ppm 0.45-0.53 (2H, m), 0.53-0.61 (2H, m), 1.94-2.07 (2H, m), 2.34-2.45 (1H, m), 3.07-3.22 (4H, m), 4.87 (2H, q, J=8.9 Hz), 6.35 (1H, d, J=2.8 Hz), 7.20 (2H, d, J=9.0 Hz), 7.34 (2H, d, J=9.0 Hz), 7.40 (2H, d, J=2.8 Hz), 12.15 (1H, br. s.).

### Example 109

### 2-[(4-Morpholin-4-yl-4-oxobutyl)sulfanyl]-3-[4-(2,2,2-trifluoroethoxy)phenyl]-3,5-dihydro-4H-pyrrolo[3,2-d]pyrimidin-4-one

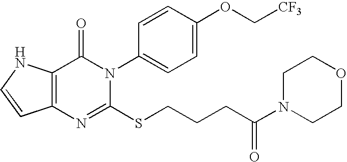

1-Ethyl-3-(3-dimethylaminopropyl)carbodiimide hydrochloride (96 mg) was added to an N,N-dimethylformamide solution (5 ml) of 4-({4-oxo-3-[4-(2,2,2-trifluoroethoxy)phenyl]-4,5-dihydro-3H-pyrrolo[3,2-d]pyrimidin-2-yl}sulfanyl)butanoic acid (214 mg) obtained by the method of Example 73, or a method pursuant to thereto, morpholine (44 mg) and 1-hydroxybenzotriazole (77 mg), and the resulting mixture was stirred for 3 days at room temperature. The solvent was distilled off under reduced pressure, and then the residue was diluted with ethyl acetate. The dilution was washed with water, a saturated aqueous solution of sodium hydrogen carbonate and saturated brine, dried over anhydrous magnesium sulfate, and then concentrated under reduced pressure. The resulting residue was recrystallized from a mixed solvent of ethyl acetate/hexane, and thus the title compound (143 mg) was obtained as a white powder.

¹H NMR (300 MHz, DMSO-d₆) δ ppm 1.72-1.91 (2H, m), 2.38 (2H, t, J=7.4 Hz), 3.06 (2H, t, J=7.2 Hz), 3.35-3.45 (4H, m), 3.47-3.60 (4H, m), 4.87 (2H, q, J=9.1 Hz), 6.34 (1H, d, J=2.7 Hz), 7.19 (2H, d, J=8.7 Hz), 7.34 (2H, d, J=8.7 Hz), 7.39 (1H, d, J=2.7 Hz), 12.12 (1H, s).

### Example 110

### 2-[(4-Oxo-4-pyrrolidin-1-ylbutyl)sulfanyl]-3-[4-(2,2,2-trifluoroethoxy)phenyl]-3,5-dihydro-4H-pyrrolo[3,2-d]pyrimidin-4-one

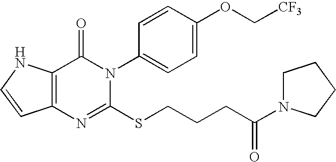

1-Ethyl-3-(3-dimethylaminopropyl)carbodiimide hydrochloride (96 mg) was added to a mixture of 4-({4-oxo-3-[4-(2,2,2-trifluoroethoxy)phenyl]-4,5-dihydro-3H-pyrrolo[3,2-d]pyrimidin-2-yl}sulfanyl)butanoic acid (214 mg) obtained by the method of Example 73, or a method pursuant to thereto, pyrrolidine (36 mg), 1-hydroxybenzotriazole (77 mg) and N,N-dimethylformamide (5 ml), and the resulting mixture was stirred for 3 days at room temperature. The solvent was distilled off under reduced pressure, and then the residue was diluted with ethyl acetate. The dilution was washed with water, a saturated aqueous solution of sodium hydrogen carbonate and saturated brine, dried over anhydrous magnesium sulfate, and then concentrated under reduced pressure. The resulting residue was recrystallized from a mixed solvent of ethyl acetate/hexane, and thus the title compound (128 mg) was obtained as a white powder.

¹H NMR (300 MHz, DMSO-d₆) δ ppm 1.61-1.90 (6H, m), 2.30 (2H, t, J=7.0 Hz), 3.07 (2H, t, J=7.2 Hz), 3.24 (2H, t, J=6.6 Hz), 3.28-3.41 (2H, m), 4.87 (2H, q, J=8.7 Hz), 6.34 (1H, s), 7.19 (2H, d, J=8.7 Hz), 7.34 (2H, d, J=8.7 Hz), 7.39 (1H, t, J=2.7 Hz), 12.12 (1H, br. s.).

### Example 111

### 2-[(2-Morpholin-4-ylethyl)sulfanyl]-3-[4-(2,2,2-trifluoroethoxy)phenyl]-3,5-dihydro-4H-pyrrolo[3,2-d]pyrimidin-4-one

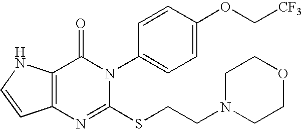

A 1 M aqueous solution of sodium hydrogen carbonate (1.2 ml) was added to a mixture of 2-thioxo-3-[4-(2,2,2-trifluoroethoxy)phenyl]-1,2,3,5-tetrahydro-4H-pyrrolo[3,2-d]pyrimidin-4-one (200 mg) obtained by the method of Example 2, or a method pursuant to thereto, 4-(2-chloroethyl)morpholine hydrochloride (109 mg), sodium iodide (88 mg) and N,N-dimethylformamide (5 ml), and the resulting mixture was stirred for 2 hours at 100° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (173 mg) was obtained as a white powder.

¹H NMR (400 MHz, DMSO-d₆) δ ppm 2.38 (4H, br. s.), 2.54 (2H, t, J=7.1 Hz), 3.19 (2H, t, J=7.1 Hz), 3.46-3.58 (4H, m), 4.88 (2H, q, J=9.0 Hz), 6.34 (1H, d, J=2.9 Hz), 7.19 (2H, d, J=8.9 Hz), 7.33 (2H, d, J=8.9 Hz), 7.39 (1H, d, J=2.9 Hz), 12.13 (1H, s).

### Example 112

### 2-[(3-Morpholin-4-ylpropyl)sulfanyl]-3-[4-(2,2,2-trifluoroethoxy)phenyl]-3,5-dihydro-4H-pyrrolo[3,2-d]pyrimidin-4-one

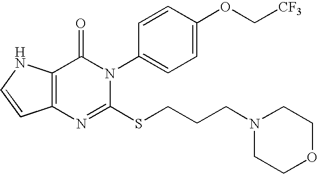

A 1 M aqueous solution of sodium hydrogen carbonate (1.2 ml) was added to a mixture of 2-thioxo-3-[4-(2,2,2-trifluoroethoxy)phenyl]-1,2,3,5-tetrahydro-4H-pyrrolo[3,2-d]pyrimidin-4-one (200 mg) obtained by the method of Example 2, or a method pursuant to thereto, 4-(3-chloropropyl)morpholine hydrochloride (117 mg), sodium iodide (88 mg) and N,N-dimethylformamide (5 ml), and the resulting mixture was stirred for 2 hours at 100° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (143 mg) was obtained as a white powder.

¹H NMR (300 MHz, DMSO-d₆) δ ppm 1.66-1.84 (2H, m), 2.25-2.37 (6H, m), 3.06 (2H, t, J=7.3 Hz), 3.48-3.63 (4H, m), 4.87 (2H, q, J=8.8 Hz), 6.31 (1H, d, J=2.8 Hz), 7.14-7.22 (2H, m), 7.29-7.36 (2H, m), 7.38 (1H, d, J=2.8 Hz), 12.12 (1H, s).

### Example 113

### N-(2-cyanoethyl)-4-({3-[4-(3-methylbutoxy)phenyl]-4-oxo-4,5-dihydro-3H-pyrrolo[3,2-d]pyrimidin-2-yl}sulfanyl)butanamide

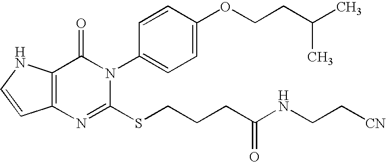

A mixture of 3-[4-(3-methylbutoxy)phenyl]-2-thioxo-1,2,3,5-tetrahydro-4H-pyrrolo[3,2-d]pyrimidin-4-one (413 mg) obtained by the method of Example 11, or a method pursuant to thereto, 4-bromo-N-(2-cyanoethyl)butanamide (307 mg) obtained by the method of Reference Example 49, or a method pursuant to thereto, potassium carbonate (359 mg) and N,N-dimethylformamide (5 ml) was stirred for 24 hours at room temperature. Subsequently, water (50 ml) was added to the reaction mixture, and the resulting mixture was extracted with ethyl acetate. The organic layer was washed with saturated brine, dried over anhydrous sodium sulfate, and then concentrated under reduced pressure. The resulting residue was purified by chromatography, and then was recrystallized from a mixed solvent of ethyl acetate/hexane. Thus, the title compound (183 mg) was obtained as a white solid.

¹H NMR (300 MHz, DMSO-d₆) δ ppm 0.96 (6H, d, J=6.4 Hz), 1.66 (2H, q, J=6.6 Hz), 1.75-1.91 (3H, m), 2.17 (2H, t, J=7.4 Hz), 2.60 (2H, t, J=6.4 Hz), 3.05 (2H, t, J=7.2 Hz), 3.24 (2H, td, J=6.4, 5.7 Hz), 4.07 (2H, t, J=6.6 Hz), 6.34 (1H, dd, J=3.1, 1.7 Hz), 7.05 (2H, d, J=9.1 Hz), 7.24 (2H, d, J=9.1 Hz), 7.38 (1H, dd, J=3.1, 2.8 Hz), 8.20 (1H, t, J=5.7 Hz), 12.09 (1H, br. s.).

### Example 114

### N-(2-cyanoethyl)-4-({4-oxo-3-[4-(4,4,4-trifluorobutoxy)phenyl]-4,5-dihydro-3H-pyrrolo[3,2-d]pyrimidin-2-yl}sulfanyl)butanamide

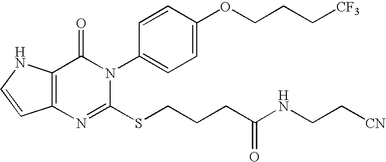

A mixture of 2-thioxo-3-[4-(4,4,4-trifluorobutoxy)phenyl]-1,2,3,5-tetrahydro-4H-pyrrolo[3,2-d]pyrimidin-4-one (351 mg) obtained by the method of Example 12, or a method pursuant to thereto, 4-bromo-N-(2-cyanoethyl)butanamide (263 mg) obtained by the method of Reference Example 49, or a method pursuant to thereto, potassium carbonate (276 mg) and N,N-dimethylformamide (5 ml), was stirred for 24 hours at room temperature. Subsequently, water (50 ml) was added to the reaction mixture, and the resulting mixture was extracted with ethyl acetate. The organic layer was washed with saturated brine, dried over anhydrous sodium sulfate, and then concentrated under reduced pressure. The resulting residue was purified by chromatography, and then was recrystallized from a mixed solvent of ethyl acetate/hexane. Thus, the title compound (219 mg) was obtained as a white solid.

¹H NMR (300 MHz, DMSO-d₆) δ ppm 1.82 (2H, tt, J=7.5, 7.2 Hz), 1.92-2.07 (2H, m), 2.17 (2H, t, J=7.5 Hz), 2.35-2.47 (2H, m), 2.60 (2H, t, J=6.4 Hz), 3.05 (2H, t, J=7.2 Hz), 3.24 (2H, td, J=6.4, 5.7 Hz), 4.12 (2H, t, J=6.2 Hz), 6.34 (1H, d, J=2.9 Hz), 7.07 (2H, d, J=9.0 Hz), 7.26 (2H, d, J=9.0 Hz), 7.38 (1H, d, J=2.9 Hz), 8.20 (1H, t, J=5.7 Hz), 12.10 (1H, s).

### Example 115

### N-(2-cyanoethyl)-4-({3-[4-(2,2-dimethylpropoxy)phenyl]-4-oxo-4,5-dihydro-3H-pyrrolo[3,2-d]pyrimidin-2-yl}sulfanyl)butanamide

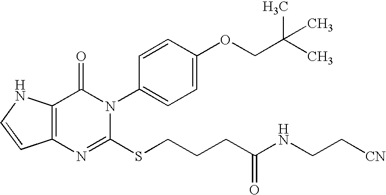

A mixture of 3-[4-(2,2-dimethylpropoxy)phenyl]-2-thioxo-1,2,3,5-tetrahydro-4H-pyrrolo[3,2-d]pyrimidin-4-one (216 mg) obtained by the method of Example 13, or a method pursuant to thereto, 4-bromo-N-(2-cyanoethyl)butanamide (175 mg) obtained by the method of Reference Example 49, or a method pursuant to thereto, potassium carbonate (193 mg) and N,N-dimethylformamide (5 ml), was stirred for 24 hours at room temperature. Subsequently, water (50 ml) was added to the reaction mixture, and the resulting mixture was extracted with ethyl acetate. The organic layer was washed with saturated brine, dried over anhydrous sodium sulfate, and then concentrated under reduced pressure. The resulting residue was purified by chromatography, and then was recrystallized from a mixed solvent of ethyl acetate/hexane. Thus, the title compound (112 mg) was obtained as a white solid.

¹H NMR (300 MHz, DMSO-d₆) δ ppm 1.04 (9H, s), 1.82 (2H, tt, J=7.4, 7.2 Hz), 2.17 (2H, t, J=7.4 Hz), 2.60 (2H, t, J=6.4 Hz), 3.05 (2H, t, J=7.2 Hz), 3.24 (2H, td, J=6.4, 5.7 Hz), 3.70 (2H, s), 6.34 (1H, dd, J=3.0, 1.9 Hz), 7.05 (2H, d, J=8.9 Hz), 7.23 (2H, d, J=8.9 Hz), 7.38 (1H, dd, J=3.0, 2.7 Hz), 8.20 (1H, t, J=5.7 Hz), 12.09 (1H, br. s.).

### Example 116

### 2-[(3-Hydroxypropyl)sulfanyl]-3-[4-(2,2,2-trifluoroethoxy)phenyl]-3,5-dihydro-4H-pyrrolo[3,2-d]pyrimidin-4-one

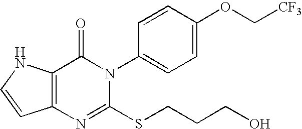

A 1 M aqueous solution of sodium hydrogen carbonate (0.5 ml) was added to a mixture of 2-thioxo-3-[4-(2,2,2-trifluoroethoxy)phenyl]-1,2,3,5-tetrahydro-4H-pyrrolo[3,2-d]pyrimidin-4-one (171 mg) obtained by the method of Example 2, or a method pursuant to thereto, 3-bromopropan-1-ol (347 mg), sodium iodide (75 mg) and N,N-dimethylformamide (5 ml), and the resulting mixture was stirred for 2 hours at 100° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (155 mg) was obtained as a white powder.

¹H NMR (300 MHz, DMSO-d₆) δ ppm 1.65-1.80 (2H, m), 3.08 (2H, t, J=7.4 Hz), 3.38-3.48 (2H, m), 4.54 (1H, t, J=5.3 Hz), 4.87 (2H, q, J=8.7 Hz), 6.35 (1H, d, J=2.7 Hz), 7.19 (2H, d, J=8.7 Hz), 7.33 (2H, d, J=8.7 Hz), 7.38 (1H, d, J=2.7 Hz), 12.12 (1H, br. s.).

### Example 117

### 2-{[4-(1H-tetrazol-5-yl)butyl]sulfanyl}-3-[4-(2,2,2-trifluoroethoxy)phenyl]-3,5-dihydro-4H-pyrrolo[3,2-d]pyrimidin-4-one

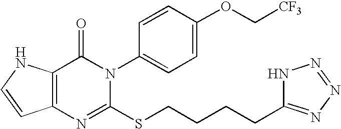

A mixture of 5-({4-oxo-3-[4-(2,2,2-trifluoroethoxy)phenyl]-4,5-dihydro-3H-pyrrolo[3,2-d]pyrimidin-2-yl}sulfanyl)pentanenitrile (150 mg) obtained in Example 87, azido(trimethyl)silane (207 mg), dibutyl(oxo)stannum (8.96 mg) and toluene (10 ml) was stirred for 4 days at 120° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by reverse phase chromatography, and then was recrystallized from ethyl acetate. Thus, the title compound (50 mg) was obtained as a white powder.

¹H NMR (300 MHz, DMSO-d₆) δ ppm 1.51-1.87 (4H, m), 2.79-2.98 (2H, m), 3.00-3.17 (2H, m), 4.77-4.99(2H, m), 6.34 (1H, br. s.), 7.18 (2H, d, J=8.0 Hz), 7.33 (2H, d, J=8.3 Hz), 7.38 (1H, br. s.), 12.12 (1H, br. s.).

### Example 118

### 2-{[5-(1H-tetrazol-5-yl)pentyl]sulfanyl}-3-[4-(2,2,2-trifluoroethoxy)phenyl]-3,5-dihydro-4H-pyrrolo[3,2-d]pyrimidin-4-one

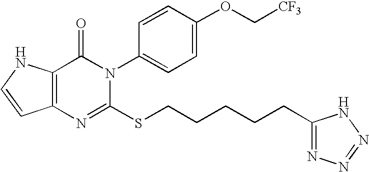

A mixture of 6-({4-oxo-3-[4-(2,2,2-trifluoroethoxy)phenyl]-4,5-dihydro-3H-pyrrolo[3,2-d]pyrimidin-2-yl}sulfanyl)hexanenitrile (150 mg) obtained in Example 88, azido(trimethyl)silane (198 mg), dibutyl(oxo)stannum (8.5 mg) and toluene (10 ml) was stirred for 4 days at 120° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by reverse phase chromatography, and then was recrystallized from ethyl acetate. Thus, the title compound (100 mg) was obtained as a white powder.

¹H NMR (300 MHz, DMSO-d₆) δ ppm 1.27-1.43 (2H, m), 1.52-1.80 (4H, m), 2.87 (2H, t, J=7.5 Hz), 3.04 (2H, t, J=7.2 Hz), 4.87 (2H, q, J=8.9 Hz), 6.34 (1H, t, J=2.2 Hz), 7.19 (2H, d, J=8.9 Hz), 7.32 (2H, d, J=8.9 Hz), 7.38 (1H, t, J=2.7 Hz), 12.12 (1H, br. s.).

### Example 119

### 2-[(4-Hydroxybutyl)sulfanyl]-3-[4-(2,2,2-trifluoroethoxy)phenyl]-3,5-dihydro-4H-pyrrolo[3,2-d]pyrimidin-4-one

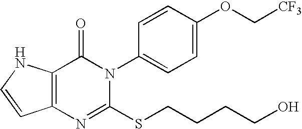

A mixture of 2-thioxo-3-[4-(2,2,2-trifluoroethoxy)phenyl]-1,2,3,5-tetrahydro-4H-pyrrolo[3,2-d]pyrimidin-4-one (171 mg) obtained by the method of Example 2, or a method pursuant to thereto, 4-bromobutan-1-ol (383 mg), sodium iodide (75 mg), triethylamine (1 ml) and N,N-dimethylformamide (5 ml) was stirred for 15 hours at 100° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by reverse phase chromatography, and thus the title compound (20 mg) was obtained as a white powder.

¹H NMR (300 MHz, DMSO-d₆) δ ppm 1.38-1.53 (2H, m), 1.54-1.68 (2H, m), 3.06 (2H, t, J=7.2 Hz), 3.34-3.42 (2H, m), 4.39 (1H, t, J=5.2 Hz), 4.87 (2H,q, J=8.9 Hz), 6.35 (1H, d, J=2.8 Hz), 7.15-7.22 (2H, m), 7.29-7.36 (2H, m), 7.38 (1H, d, J=2.8 Hz), 12.11 (1H, s).

### Example 120

### 3-({4-Oxo-3-[4-(2,2,2-trifluoroethoxy)phenyl]-4,5-dihydro-3h-pyrrolo[3,2-d]pyrimidin-2-yl}sulfanyl)propane-1-sulfonamide

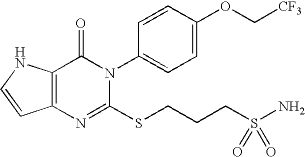

A mixture of 2-thioxo-3-[4-(2,2,2-trifluoroethoxy)phenyl]-1,2,3,5-tetrahydro-4H-pyrrolo[3,2-d]pyrimidin-4-one (682 mg) obtained by the method of Example 2, or a method pursuant to thereto, 3-chloropropane-1-sulfonamide (946 mg) obtained by a method described in a published document, Journal of Organic Chemistry (J. Org. Chem.), Vol. 11, p. 2162 (1987), or a method pursuant to thereto, triethylamine (1.4 ml) and N,N-dimethylformamide (30 ml) was stirred for 15 hours at 100° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of methanol/ethyl acetate. Thus, the title compound (806 mg) was obtained as a white powder.

¹H NMR (300 MHz, DMSO-d₆) δ ppm 2.01-2.12 (2H, m), 2.98-3.09 (2H, m), 3.18 (2H, t, J=7.0 Hz), 4.87 (2H, q, J=8.9 Hz), 6.36 (1H, d, J=2.8 Hz), 6.80 (2H, br. s.), 7.20 (2H, d, J=9.0 Hz), 7.35 (2H, d, J=9.0 Hz), 7.40 (1H, d, J=2.8 Hz), 12.14 (1H, br. s.).

### Example 121

### 2-(Methylsulfanyl)-3-[4-(2,2,2-trifluoroethoxy)phenyl]-3,5-dihydro-4H-pyrrolo[3,2-d]pyrimidin-4-one

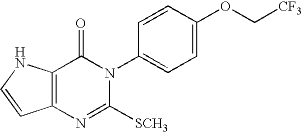

A 1 M aqueous solution of sodium hydrogen carbonate (0.5 ml) was added to a solution of 2-thioxo-3-[4-(2,2,2-trifluoroethoxy)phenyl]-1,2,3,5-tetrahydro-4H-pyrrolo[3,2-d]pyrimidin-4-one (171 mg) obtained by the method of Example 2, or a method pursuant to thereto, iodomethane (126 μl) and N,N-dimethylformamide (5 ml), and the resulting mixture was stirred for 2 hours at 100° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (135 mg) was obtained as a white powder.

¹H NMR (300 MHz, DMSO-d₆) δ ppm 2.41 (3H, s), 4.87 (2H, q, J=8.9 Hz), 6.37 (1H, d, J=2.8 Hz), 7.19 (2H, d, J=9.0 Hz), 7.34 (2H, d, J=9.0 Hz), 7.39 (1H, d, J=2.8 Hz), 12.12 (1H, s).

### Example 122 2-[(4-Oxopentyl)sulfanyl]-3-[4-(2,2,2-trifluoroethoxy)phenyl]-3,5-dihydro-4H-pyrrolo[3,2-d]pyrimidin-4-one

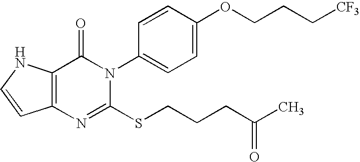

A 1 M aqueous solution of sodium hydrogen carbonate (2.0 ml) was added to a mixture of 2-thioxo-3-[4-(2,2,2-trifluoroethoxy)phenyl]-1,2,3,5-tetrahydro-4H-pyrrolo[3,2-d]pyrimidin-4-one (682 mg) obtained by the method of Example 2, or a method pursuant to thereto, 5-chloropentan-2-one (242 mg) and N,N-dimethylformamide (20 ml), and the resulting mixture was stirred for 24 hours at 100° C. To the reaction mixture, 5-chloropentan-2-one (242 mg) was added, and the resulting mixture was stirred for 24 hours at 100° C. The reaction mixture was returned to room temperature, and diluted with ethyl acetate. The dilution was washed with water, a saturated aqueous solution of sodium hydrogen carbonate and saturated brine, dried over anhydrous magnesium sulfate, and then concentrated under reduced pressure. The resulting residue was purified by chromatography, and thus the title compound (285 mg) was obtained as a white powder.

¹H NMR (300 MHz, DMSO-d₆) δ ppm 1.71-1.85 (2H, m), 2.06 (3H, s), 2.46-2.56 (2H, m), 3.02 (2H, t, J=7.2 Hz), 4.87 (2H, q, J=8.8 Hz), 6.34 (1H, d, J=2.8 Hz), 7.14-7.23 (2H, m), 7.29-7.37 (2H, m), 7.39 (1H, d, J=2.6 Hz), 12.12 (1H, br. s.).

### Example 123

### 2-[(4-Hydroxy-4-methylpentyl)sulfanyl]-3-[4-(2,2,2-trifluoroethoxy)phenyl]-3,5-dihydro-4H-pyrrolo[3,2-d]pyrimidin-4-one

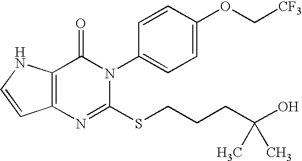

To a tetrahydrofuran solution (5 ml) of 2-[(4-oxopentyl)sulfanyl]-3-[4-(2,2,2-trifluoroethoxy)phenyl]-3,5-dihydro-4H-pyrrolo[3,2-d]pyrimidin-4-one (100 mg) obtained in Example 122, a 3 M methyl magnesium bromide/diethyl ether solution (0.23 ml) was added dropwise at 0° C., and the resulting mixture was stirred for one hour at room temperature. The reaction was stopped with a saturated aqueous solution of ammonium chloride, and the reaction liquid was extracted with ethyl acetate. The organic layer was washed with water and saturated brine, dried over anhydrous magnesium sulfate, and then concentrated under reduced pressure. The resulting residue was purified by chromatography, and thus the title compound (81 mg) was obtained as a white powder.

¹HNMR (300 MHz, DMSO-d₆) δ ppm 1.04 (6H, s), 1.34-1.44 (2H, m), 1.56-1.70 (2H, m), 3.04 (2H, t, J=7.3 Hz), 4.13 (1H, s), 4.87 (2H, q, J=8.9 Hz), 6.34 (1H, d, J=2.6 Hz), 7.14-7.23 (2H, m), 7.29-7.36 (2H, m), 7.38 (1H, d, J=1.5 Hz), 12.11 (1H, s).

### Example 124

### N-(methylsulfonyl)-4-({4-oxo-3-[4-(2,2,2-trifluoroethoxy)phenyl]-4,5-dihydro-3H-pyrrolo[3,2-d]pyrimidin-2-yl}sulfanyl)butanamide

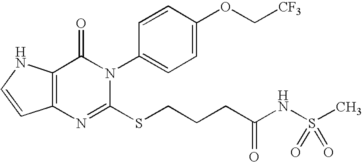

2-Methyl-6-nitrobenzoic acid anhydride (207 mg) was added to a mixture of 4-({4-oxo-3-[4-(2,2,2-trifluoroethoxy)phenyl]-4,5-dihydro-3H-pyrrolo[3,2-d]pyrimidin-2-yl}sulfanyl)butanoic acid (214 mg) obtained in Example 73, methanesulfonamide (52 mg), triethylamine (209 μl), 4-dimethylaminopyridine (61 mg) and acetonitrile (10 ml), and the resulting mixture was stirred for 15 hours at room temperature. The solvent was distilled off under reduced pressure, and the residue was diluted with water. Then, the dilution was extracted with ethyl acetate. The organic layer was washed with 1 M hydrochloric acid and saturated brine, dried over anhydrous magnesium sulfate, and then concentrated under reduced pressure. The resulting residue was purified by chromatography, and was recrystallized from a mixed solvent of ethyl acetate/hexane. Thus, the title compound (35 mg) was obtained as a white powder.

¹H NMR (300 MHz, DMSO-d₆) δ ppm 1.85 (2H, m), 2.35 (2H, t, J=7.3 Hz), 3.06 (2H, t, J=7.1 Hz), 3.20 (3H, s), 4.87 (2H, q, J=8.9 Hz), 6.36 (1H, dd, J=2.6, 2.1 Hz), 7.19 (2H, d, J=9.0 Hz), 7.29-7.37 (2H, m), 7.39 (1H, t, J=2.9 Hz), 11.68 (1H, br. s.), 12.13 (1H, br. s.).

### Example 125

N-{[3-({4-oxo-3-[4-(2,2,2-trifluoroethoxy)phenyl]-4,5-dihydro-3H-pyrrolo[3,2-d]pyrimidin-2-yl}sulfanyl)propyl]sulfonyl}cyclopropanecarboxamide

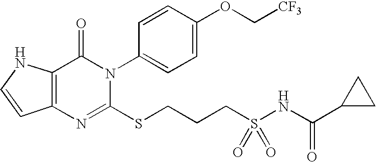

2-Methyl-6-nitrobenzoic acid anhydride (207 mg) was added to a mixture of 3-({4-oxo-3-[4-(2,2,2-trifluoroethoxy)phenyl]-4,5-dihydro-3h-pyrrolo[3,2-D]pyrimidin-2-yl}sulfanyl)propane-1-sulfonamide (231 mg) obtained in Example 120, cyclopropanecarboxylic acid (52 mg), triethylamine (209 μl), 4-dimethylaminopyridine (61 mg) and acetonitrile (10 ml), and the resulting mixture was stirred for 15 hours at room temperature. The solvent was distilled off under reduced pressure, and the mixture was diluted with water, and then was extracted with ethyl acetate. The organic layer was washed with 1 M hydrochloric acid and saturated brine, dried over anhydrous magnesium sulfate, and then concentrated under reduced pressure. The resulting residue was purified by chromatography, and thus the title compound (60 mg) was obtained as a white powder.

¹H NMR (300 MHz, DMSO-d₆) δ ppm 0.68-0.85 (4H, m), 1.59-1.72 (1H, m), 1.95-2.13 (2H, m), 3.15 (2H, t, J=6.8 Hz), 3.39-3.49 (2H, m), 4.87 (2H, q, J=8.8 Hz), 6.34 (1H, t, J=2.3 Hz), 7.20 (2H, d, J=8.9 Hz), 7.34 (2H, d, J=8.9 Hz), 7.40 (1H, t, J=2.8 Hz), 11.90 (1H, s), 12.15 (1H, br. s.).

### Example 126

### 2-[(Cyclopropylmethyl)sulfanyl]-3-[4-(2,2,2-trifluoroethoxy)phenyl]-3,5-dihydro-4H-pyrrolo[3,2-d]pyrimidin-4-one

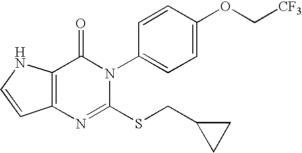

A 1 M aqueous solution of sodium hydrogen carbonate (0.5 ml) was added to a mixture of 2-thioxo-3-[4-(2,2,2-trifluoroethoxy)phenyl]-1,2,3,5-tetrahydro-4H-pyrrolo[3,2-d]pyrimidin-4-one (171 mg) obtained by the method of Example 2, or a method pursuant to thereto, (bromomethyl)cyclopropane (68 mg), sodium iodide (75 mg) and N,N-dimethylformamide (5 ml), and the resulting mixture was stirred for 2 hours at 100° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (170 mg) was obtained as a white powder.

¹H NMR (300 MHz, DMSO-d₆) δ ppm 0.20-0.26 (2H, m), 0.45-0.54 (2H, m), 0.96-1.13 (1H, m), 2.99 (2H, d, J=7.3 Hz), 4.88 (2H, q, J=9.0 Hz), 6.35 (1H, d, J=2.8 Hz), 7.15-7.23 (2H, m), 7.29-7.37 (2H, m), 7.38 (1H, d, J=2.8 Hz), 12.11 (1H, s).

### Example 127

### 2-[(2-Hydroxyethyl)sulfanyl]-3-[4-(2,2,2-trifluoroethoxy)phenyl]-3,5-dihydro-4H-pyrrolo[3,2-d]pyrimidin-4-one

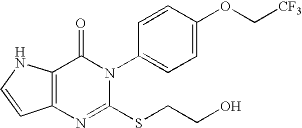

A 1 M aqueous solution of sodium hydrogen carbonate (0.5 ml) was added to a mixture of 2-thioxo-3-[4-(2,2,2-trifluoroethoxy)phenyl]-1,2,3,5-tetrahydro-4H-pyrrolo[3,2-d]pyrimidin-4-one (171 mg) obtained by the method of Example 2, or a method pursuant to thereto, 2-bromoethanol (63 mg), sodium iodide (75 mg) and N,N-dimethylformamide (5 ml), and the resulting mixture was stirred for 2 hours at 100° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (127 mg) was obtained as a white powder.

¹H NMR (300 MHz, DMSO-d₆) δ ppm 3.16 (2H, t, J=6.4 Hz), 3.59 (2H, q, J=6.2 Hz), 4.79-4.96 (3H, m), 6.32-6.37 (1H, m), 7.19 (2H, d, J=9.1 Hz), 7.32 (2H, d, J=9.1 Hz), 7.39 (1H, t, J=2.8 Hz), 12.12 (1H, br. s.).

### Example 128

### Ethyl 3-({4-oxo-3-[4-(2,2,2-trifluoroethoxy)phenyl]-4,5-dihydro-3H-pyrrolo[3,2-d]pyrimidin-2-yl}sulfanyl)propanoate

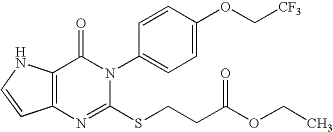

A mixture of 2-thioxo-3-[4-(2,2,2-trifluoroethoxy)phenyl]-1,2,3,5-tetrahydro-4H-pyrrolo[3,2-d]pyrimidin-4-one (341 mg) obtained by the method of Example 2, or a method pursuant to thereto, ethyl 3-bromopropanoate (501 mg), sodium iodide (150 mg), triethylamine (120 μl) and N,N-dimethylformamide (30 ml) was stirred for 15 hours at 100° C. The reaction mixture was returned to room temperature, and then the solvent was distilled off under reduced pressure. The residue was diluted with ethyl acetate, and the dilution was washed with 1 M hydrochloric acid, water and saturated brine, dried over anhydrous magnesium sulfate, and then concentrated under reduced pressure. The resulting residue was purified by chromatography, and then was recrystallized from a mixed solvent of methanol/ethyl acetate. Thus, the title compound (295 mg) was obtained as a white powder.

¹H NMR (300 MHz, DMSO-d₆) δ ppm 1.16 (3H, t, J=7.2 Hz), 2.71 (2H, t, J=6.9 Hz), 3.24 (2H, t, J=6.9 Hz), 4.05 (2H, q, J=7.2 Hz), 4.87 (2H, q, J=8.8 Hz), 6.36 (1H, d, J=2.7 Hz), 7.19 (2H, d, J=8.7 Hz), 7.32 (2H, d, J=8.7 Hz), 7.40 (1H, d, J=3.0 Hz), 12.14 (1H, br. s.).

### Example 129

### 2-[(Difluoromethyl)sulfanyl]-3-[4-(2,2,2-trifluoroethoxy)phenyl]-3,5-dihydro-4H-pyrrolo[3,2-d]pyrimidin-4-one

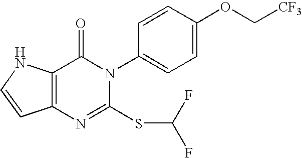

A 1 M aqueous solution of sodium hydrogen carbonate (1.0 ml) was added to a mixture of 2-thioxo-3-[4-(2,2,2-trifluoroethoxy)phenyl]-1,2,3,5-tetrahydro-4H-pyrrolo[3,2-d]pyrimidin-4-one (341 mg) obtained by the method of Example 2, or a method pursuant to thereto, difluoro(iodo)methane (500 mg) and N,N-dimethylformamide (10 ml), and the resulting mixture was stirred for 15 hours at 100° C. Furthermore, a N,N-dimethylformamide solution (5 ml) of difluoro(iodo)methane (500 mg) was added and the mixture was stirred for 15 hours at 100° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of hexane/diisopropyl ether. Thus, the title compound (25 mg) was obtained as a white powder.

¹H NMR (300 MHz, DMSO-d₆) δ ppm 4.89 (2H, q, J=9.0 Hz), 6.40-6.45 (1H, m), 7.23 (2H, d, J=9.1 Hz), 7.39-7.49 (3H, m), 7.82 (1H, t, J=25.2 Hz), 12.34 (1H, br. s.).

### Example 130

### 2-{[2-Hydroxy-3-(1-methylethoxy)propyl]sulfanyl}-3-[4-(2,2,2-trifluoroethoxy)phenyl]-3,5-dihydro-4H-pyrrolo[3,2-d]pyrimidin-4-one

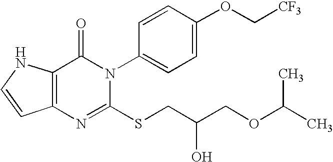

A mixture of 2-thioxo-3-[4-(2,2,2-trifluoroethoxy)phenyl]-1,2,3,5-tetrahydro-4H-pyrrolo[3,2-d]pyrimidin-4-one (341 mg) obtained by the method of Example 2, or a method pursuant to thereto, 2-[(1-methylethoxy)methyl]oxirane (116 mg), sodium iodide (150 mg), triethylamine (120 μl) and N,N-dimethylformamide (10 ml) was stirred for 15 hours at 100° C. The reaction mixture was returned to room temperature, and then was concentrated under reduced pressure. The residue was diluted with ethyl acetate, and the dilution was washed with water and saturated brine, dried over anhydrous magnesium sulfate, and then concentrated under reduced pressure. The resulting residue was purified by chromatography, and then was recrystallized from a mixed solvent of hexane/diisopropyl ether. Thus, the title compound (230 mg) was obtained as a white powder.

¹H NMR (300 MHz, DMSO-d₆) δ ppm 1.06 (6H, d, J=6.1 Hz), 3.04 (1H, dd, J=12.9, 7.6 Hz), 3.22-3.41 (3H, m), 3.48-3.59 (1H, m), 3.68-3.81 (1H, m), 4.87 (2H, q, J=8.8 Hz), 5.05 (1H, d, J=5.3 Hz), 6.33 (1H, t, J=2.5 Hz), 7.20 (2H, d, J=9.0 Hz), 7.32 (2H, d, J=9.0 Hz), 7.38 (1H, t, J=3.0 Hz), 12.11 (1H, br. s.).

### Example 131

### 2-[(3-Hydroxy-3-methylbutyl)sulfanyl]-3-[4-(2,2,2-trifluoroethoxy)phenyl]-3,5-dihydro-4H-pyrrolo[3,2-d]pyrimidin-4-one

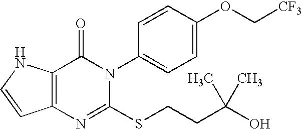

To a tetrahydrofuran solution (10 ml) of ethyl 3-({4-oxo-3-[4-(2,2,2-trifluoroethoxy)phenyl]-4,5-dihydro-3H-pyrrolo[3,2-d]pyrimidin-2-yl}sulfanyl)propanoate (200 mg) obtained in Example 128, a 1 M methyl magnesium bromide/tetrahydrofuran solution (1.87 ml) was added dropwise at room temperature, and the resulting mixture was stirred for 15 hours at room temperature. The reaction was stopped with a saturated aqueous solution of ammonium chloride, and the reaction liquid was extracted with ethyl acetate. The organic layer was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (130 mg) was obtained as a white powder.

¹H NMR (300 MHz, DMSO-d₆) δ ppm 1.11 (6H, s), 1.56-1.68 (2H, m), 2.98-3.11 (2H, m), 4.33 (1H, s), 4.87 (2H, q, J=9.0 Hz), 6.33 (1H, d, J=3.0 Hz), 7.18 (2H, d, J=9.1 Hz), 7.31 (2H, d, J=9.1 Hz), 7.38 (1H, d, J=2.7 Hz), 12.10 (1H, s).

### Example 132

### 2-[(3-Methoxypropyl)sulfanyl]-3-[4-(2,2,2-trifluoroethoxy)phenyl]-3,5-dihydro-4H-pyrrolo[3,2-d]pyrimidin-4-one

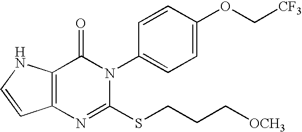

A 1 M aqueous solution of sodium hydrogen carbonate (1.0 ml) was added to a mixture of 2-thioxo-3-[4-(2,2,2-trifluoroethoxy)phenyl]-1,2,3,5-tetrahydro-4H-pyrrolo[3,2-d]pyrimidin-4-one (341 mg) obtained by the method of Example 2, or a method pursuant to thereto, 1-bromo-3-methoxypropane (153 mg), sodium iodide (150 mg) and N,N-dimethylformamide (10 ml), and the resulting mixture was stirred for 2 hours at 100° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (280 mg) was obtained as a white powder.

¹H NMR (300 MHz, DMSO-d₆) δ ppm 1.74-1.88 (2H, m), 3.06 (2H, t, J=7.4 Hz), 3.21 (3H, s), 3.35 (2H, t, J=6.1 Hz), 4.87 (2H, q, J=9.0 Hz), 6.35 (1H, d, J=2.7 Hz), 7.19 (2H, d, J=8.7 Hz), 7.33 (2H, d, J=8.7 Hz), 7.38 (1H, d, J=3.0 Hz), 12.12 (1H, s).

### Example 133

### 2-{[3-(4-Fluorophenoxy)propyl]sulfanyl}-3-[4-(2,2,2-trifluoroethoxy)phenyl]-3,5-dihydro-4H-pyrrolo[3,2-d]pyrimidin-4-one

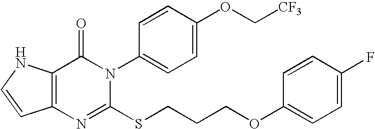

A 1 M aqueous solution of sodium hydrogen carbonate (1.0 ml) was added to a mixture of 2-thioxo-3-[4-(2,2,2-trifluoroethoxy)phenyl]-1,2,3,5-tetrahydro-4H-pyrrolo[3,2-d]pyrimidin-4-one (341 mg) obtained by the method of Example 2, or a method pursuant to thereto, 1-(3-chloropropoxy)-4-fluorobenzene (189 mg), sodium iodide (150 mg) and N,N-dimethylformamide (10 ml), and the resulting mixture was stirred for 2 hours at 100° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (260 mg) was obtained as a white powder.

¹H NMR (300 MHz, DMSO-d₆) δ ppm 2.04 (2H, m), 3.18 (2H, t, J=7.2 Hz), 3.99 (2H, t, J=6.2 Hz), 4.87 (2H, q, J=9.0 Hz), 6.33 (1H, d, J=2.7 Hz), 6.87-6.96 (2H, m), 7.04-7.14 (2H, m), 7.19 (2H, d, J=9.1 Hz), 7.34 (2H, d, J=9.1 Hz), 7.39 (1H, d, J=3.0 Hz), 12.12 (1H, s).

### Example 134

### 2-{[4-(4-Hydroxy-4-methylpiperidin-1-yl)-4-oxobutyl]sulfanyl}-3-[4-(2,2,2-trifluoroethoxy)phenyl]-3,5-dihydro-4H-pyrrolo[3,2-d]pyrimidin-4-one

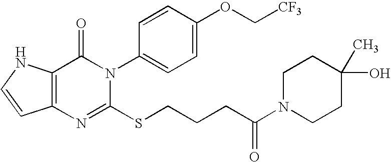

1-Ethyl-3-(3-dimethylaminopropyl)carbodiimide hydrochloride (115 mg) was added to a mixture of 4-({4-oxo-3-[4-(2,2,2-trifluoroethoxy)phenyl]-4,5-dihydro-3H-pyrrolo[3,2-d]pyrimidin-2-yl}sulfanyl)butanoic acid (214 mg) obtained by the method of Example 73, or a method pursuant to thereto, 4-methylpiperidin-4-ol hydrochloride (58 mg) obtained by a method described in a published document, Journal of the American Chemical Society (J. Am. Chem. Soc.), Vol. 115, p. 7250 (1993), or a method pursuant to thereto, 1-hydroxybenzotiazole (77 mg) and N,N-dimethylformamide (10 ml), and the resulting mixture was stirred for 15 hours at room temperature. The solvent was distilled off under reduced pressure, and then the residue was diluted with ethyl acetate. The dilution was washed with water, a saturated aqueous solution of sodium hydrogen carbonate and saturated brine, dried over anhydrous magnesium sulfate, and then concentrated under reduced pressure. The resulting residue was purified by chromatography and reverse phase chromatography, and thus the title compound (89 mg) was obtained as a white powder.

¹H NMR (300 MHz, DMSO-d₆) δ ppm 1.09 (3H, s), 1.21-1.48 (4H, m), 1.80 (2H, t, J=7.1 Hz), 2.37 (2H, t, J=7.3 Hz), 2.92-3.09 (3H, m), 3.21-3.34 (1H, m), 3.43-3.53 (1H, m), 3.84-3.96 (1H, m), 4.35 (1H, br. s.), 4.87 (2H, q, J=8.9 Hz), 6.34 (1H, d, J=2.1 Hz), 7.19 (2H, d, J=8.9 Hz), 7.33 (2H, d, J=8.9 Hz), 7.39 (1H, br. s.), 12.12 (1H, br. s.).

### Example 135

### 2-{[4-(1,1-Dioxidothiomorpholin-4-yl)-4-oxobutyl]sulfanyl}-[4-(2,2,2-trifluoroethoxy)phenyl]-3,5-dihydro-4H-pyrrolo[3,2-d]pyrimidin-4-one

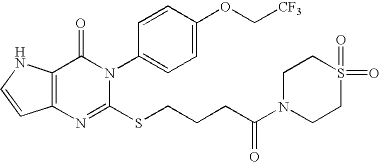

1-Ethyl-3-(3-dimethylaminopropyl)carbodiimide hydrochloride (115 mg) was added to a mixture of 4-({4-oxo-3-[4-(2,2,2-trifluoroethoxy)phenyl]-4,5-dihydro-3H-pyrrolo [3,2-d]pyrimidin-2-yl}sulfanyl)butanoic acid (214 mg) obtained by the method of Example 73, or a method pursuant to thereto, thiomorpholine 1,1-dioxide (68 mg), 1-hydroxybenzotriazole (77 mg) and N,N-dimethylformamide (10 ml), and the resulting mixture was stirred for 15 hours at room temperature. The solvent was distilled off under reduced pressure, and then the residue was diluted with ethyl acetate. The dilution was washed with water, a saturated aqueous solution of sodium hydrogen carbonate and saturated brine, dried over anhydrous magnesium sulfate, and then concentrated under reduced pressure. The resulting residue was purified by chromatography and reverse phase chromatography, and thus the title compound (63 mg) was obtained as a white powder.

¹H NMR (300 MHz, DMSO-d₆) δ ppm 1.77-1.91 (2H, m), 2.44-2.53 (2H, m), 3.01-3.11 (4H, m), 3.13-3.25 (2H, m), 3.75-3.90 (4H, m), 4.87 (2H, q, J=8.9 Hz), 6.31-6.36 (1H, m), 7.17-7.21 (2H, m), 7.32-7.36 (2H, m), 7.37-7.41 (1H, m), 12.13 (1H, br. s.).

### Example 136

### 2-[(4-Oxo-4-thiomorpholin-4-ylbutyl)sulfanyl]-3-[4-(2,2,2-trifluoroethoxy)phenyl]-3,5-dihydro-4H-pyrrolo[3,2-d]pyrimidin-4-one

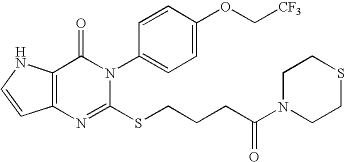

1-Ethyl-3-(3-dimethylaminopropyl)carbodiimide hydrochloride (144 mg) was added to a mixture of 4-({4-oxo-3-[4-(2,2,2-trifluoroethoxy)phenyl]-4,5-dihydro-3H-pyrrolo[3,2-d]pyrimidin-2-yl}sulfanyl)butanoic acid (215 mg) obtained by the method of Example 73, or a method pursuant to thereto, thiomorpholine (103 mg), 1-hydroxybenzotriazole (77 mg) and N,N-dimethylformamide (5 ml), and the resulting mixture was stirred for 15 hours at room temperature. The solvent was distilled off under reduced pressure, and then the residue was diluted with ethyl acetate. The dilution was washed with water, a saturated aqueous solution of sodium hydrogen carbonate and saturated brine, dried over anhydrous magnesium sulfate, and then concentrated under reduced pressure. The resulting residue was recrystallized from a mixed solvent of methanol/ethyl acetate, and thus the title compound (150 mg) was obtained as a white powder.

¹H NMR (300 MHz, DMSO-d₆) δ ppm 1.75-1.88 (2H, m), 2.39 (2H, t, J=7.3 Hz), 2.47-2.52 (2H, m), 2.54-2.61 (2H, m), 3.06 (2H, t, J=7.3 Hz), 3.61-3.72 (4H, m), 4.87 (2H, q, J=8.9 Hz), 6.35 (1H, d, J=2.8 Hz), 7.19 (2H, d, J=8.9 Hz), 7.34 (2H, d, J=8.9 Hz), 7.39 (1H, d, J=2.8 Hz), 12.12 (1H, s).

### Example 137

### 2-{[3-(1-Methylethoxy)propyl]sulfanyl}-3-[4-(2,2,2-trifluoroethoxy)phenyl]-3,5-dihydro-4H-pyrrolo[3,2-d]pyrimidin-4-one

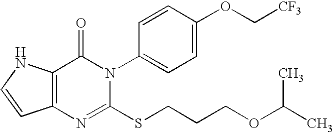

A 1 M aqueous solution of sodium hydrogen carbonate (0.58 ml) was added to a mixture of 2-thioxo-3-[4-(2,2,2-trifluoroethoxy)phenyl]-1,2,3,5-tetrahydro-4H-pyrrolo[3,2-d]pyrimidin-4-one (200 mg) obtained by the method of Example 2, or a method pursuant to thereto, 3-(1-methylethoxy)propyl 4-methylbenzenesulfonate (158 mg) obtained by a method described in a published document, Canadian Journal of Chemistry (Can. J. Chem.), Vol. 33, p. 1207 (1955), sodium iodide (87 mg) and N,N-dimethylformamide (10 ml), and the resulting mixture was stirred for 3 hours at 100° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (201 mg) was obtained as a white powder.

¹HNMR (300 MHz, DMSO-d₆) δ ppm 1.04 (6H, d, J=6.2 Hz), 1.72-1.84 (2H, m), 3.07 (2H, t J=7.5 Hz), 3.38 (2H, t, J=6.1 Hz), 3.49 (1H, spt, J=6.2 Hz), 4.87 (2H, q, J=9.0 Hz), 6.35 (1H, d, J=2.8 Hz), 7.19 (2H, d, J=9.0 Hz), 7.33 (2H, d, J=9.0 Hz), 7.38 (1H, d, J=2.8 Hz), 12.12 (1H, s).

### Example 138

### 3-[4-(Cyclopropylmethoxy)phenyl]-2-{[3-(1-methylethoxy)propyl]sulfanyl}-3,5-dihydro-4H-pyrrolo[3,2-d]pyrimidin-4-one

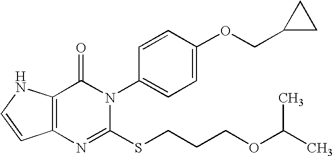

A 1 M aqueous solution of sodium hydrogen carbonate (0.64 ml) was added to a mixture of 3-[4-(cyclopropylmethoxy)phenyl]-2-thioxo-1,2,3,5-tetrahydro-4H-pyrrolo[3,2-d]pyrimidin-4-one (200 mg) obtained by the method of Example 6, or a method pursuant to thereto, 3-(1-methylethoxy)propyl 4-methylbenzenesulfonate (174 mg) obtained by a method described in a published document, Canadian Journal of Chemistry (Can. J. Chem.), Vol. 33, p. 1207 (1955), sodium iodide (96 mg) and N,N-dimethylformamide (10 ml), and the resulting mixture was stirred for 3 hours at 100° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (221 mg) was obtained as a white powder.

¹H NMR (300 MHz, DMSO-d₆) δ ppm 0.32-0.39 (2H, m), 0.56-0.64 (2H, m), 1.06 (6H, d, J=6.3 Hz), 1.21-1.33 (1H, m), 1.71-1.84 (2H, m), 3.06 (2H, t, J=7.3 Hz), 3.38 (2H, t, J=6.2 Hz), 3.49 (1H, spt, J=6.1 Hz), 3.89 (2H, d, J=7.2 Hz), 6.34 (1H, d, J=2.8 Hz), 7.04 (2H, d, J=8.9 Hz), 7.23 (2H, d, J=8.9 Hz), 7.37 (1H, d, J=2.8 Hz), 12.10 (1H, s).

### Example 139

### 3-[4-(Cyclopropylmethoxy)phenyl]-2-[(3-ethoxypropyl)sulfanyl]-3,5-dihydro-4H-pyrrolo[3,2-d]pyrimidin-4-one

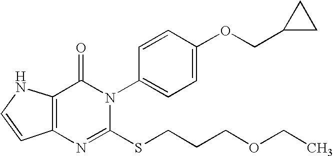

A 1 M aqueous solution of sodium hydrogen carbonate (0.64 ml) was added to a mixture of 3-[4-(cyclopropylmethoxy)phenyl]-2-thioxo-1,2,3,5-tetrahydro-4H-pyrrolo[3,2-d]pyrimidin-4-one (200 mg) obtained by the method of Example 6, or a method pursuant to thereto, 3-ethoxypropyl 4-methylbenzenesulfonate (165 mg) obtained by a method described in a published document, Canadian Journal of Chemistry (Can. J. Chem.), Vol. 33, p. 1207 (1955), or a method pursuant to thereto, sodium iodide (96 mg) and N,N-dimethylformamide (10 ml), and the resulting mixture was stirred for 3 hours at 100° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (169 mg) was obtained as a white powder.

¹H NMR (300 MHz, DMSO-d₆) δ ppm 0.32-0.40 (2H, m), 0.56-0.65 (2H, m), 1.08 (3H, t, J=7.1 Hz), 1.21-1.34 (1H, m), 1.74-1.87 (2H, m), 3.06 (2H, t, J=7.3 Hz), 3.34-3.42 (4H, m), 3.89 (2H, d, J=7.0 Hz), 6.34 (1H, d, J=2.8 Hz), 7.04 (2H, d, J=8.9 Hz), 7.23 (2H, d, J=8.9 Hz), 7.38 (1H, d), 12.10 (1H, s).

### Example 140

### 3-[4-(Cyclopropylmethoxy)-3-fluorophenyl]-2-[(3-ethoxypropyl)sulfanyl]-3,5-dihydro-4H-pyrrolo[3,2-d]pyrimidin-4-one

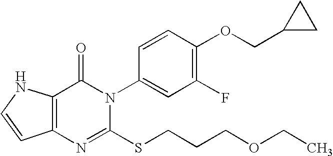

A 1 M aqueous solution of sodium hydrogen carbonate (0.6 ml) was added to a mixture of 3-[4-(cyclopropylmethoxy)-3-fluorophenyl]-2-sulfanyl-3,5-dihydro-4H-pyrrolo[3,2-d]pyrimidin-4-one (200 mg) obtained by the method of Example 50, or a method pursuant to thereto, 3-ethoxypropyl 4-methylbenzenesulfonate (155 mg) obtained by a method described in a published document, Canadian Journal of Chemistry (Can. J. Chem.), Vol. 33, p. 1207 (1955), or a method pursuant to thereto, sodium iodide (90 mg) and N,N-dimethylformamide (10 ml), and the resulting mixture was stirred for 3 hours at 100° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (195 mg) was obtained as a white powder.

¹H NMR (300 MHz, DMSO-d₆) δ ppm 0.33-0.41 (2H, m), 0.58-0.66 (2H, m), 1.08 (3H, t, J=7.0 Hz), 1.22-1.37 (1H, m), 1.74-1.89 (2H, m), 3.07 (2H, t, J=7.3 Hz), 3.34-3.43 (4H, m), 3.98 (2H, d, J=6.8 Hz), 6.35 (1H, d, J=2.8 Hz), 7.09-7.17 (1H, m), 7.25 (1H, dd, J=9.0, 8.9 Hz), 7.33-7.43 (2H, m), 12.13 (1H, s).

### Example 141

### 3-[4-(Cyclopropylmethoxy)phenyl]-2-{[3-(2-methoxyethoxy)propyl]sulfanyl}-3,5-dihydro-4H-pyrrolo[3,2-d]pyrimidin-4-one

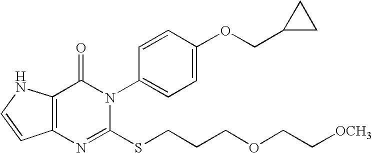

A 1 M aqueous solution of sodium hydrogen carbonate (0.64 ml) was added to a mixture of 3-[4-(cyclopropylmethoxy)phenyl]-2-thioxo-1,2,3,5-tetrahydro-4H-pyrrolo[3,2-d]pyrimidin-4-one (200 mg) obtained by the method of Example 6, or a method pursuant to thereto, 1-bromo-3-(2-methoxyethoxy)propane (126 mg), sodium iodide (96 mg) and N,N-dimethylformamide (10 ml), and the resulting mixture was stirred for 15 hours at 100° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (156 mg) was obtained as a white powder.

¹H NMR (300 MHz, DMSO-d₆) δ ppm 0.32-0.39 (2H, m), 0.56-0.65 (2H, m), 1.21-1.32(1H, m), 1.74-1.87 (2H, m), 3.06 (2H, t, J=7.2 Hz), 3.22 (3H, s), 3.38-3.49 (6H, m), 3.88 (2H, d, J=7.2 Hz), 6.35 (1H, d, J=2.7 Hz), 7.04 (2H, d, J=8.5 Hz), 7.23 (2H, d, J=8.5 Hz), 7.38 (1H, d, J=2.7 Hz), 12.10 (1H, br. s.).

### Example 142

### 3-[4-(Cyclopropylmethoxy)phenyl]-2-{[2-(2-ethoxyethoxy)ethyl]sulfanyl}-3,5-dihydro-4H-pyrrolo[3,2-d]pyrimidin-4-one

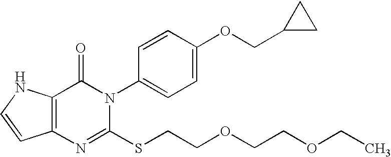

A 1 M aqueous solution of sodium hydrogen carbonate (0.64 ml) was added to a mixture of 3-[4-(cyclopropylmethoxy)phenyl]-2-thioxo-1,2,3,5-tetrahydro-4H-pyrrolo[3,2-d]pyrimidin-4-one (200 mg) obtained by the method of Example 6, or a method pursuant to thereto, 1-bromo-2-(2-ethoxyethoxy)ethane (126 mg), sodium iodide (96 mg) and N,N-dimethylformamide (10 ml), and the resulting mixture was stirred for 15 hours at 100° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (183 mg) was obtained as a white powder.

¹H NMR (300 MHz, DMSO-d₆) δ ppm 0.32-0.39 (2H, m), 0.56-0.64 (2H, m), 1.06 (3H, t, J=7.0 Hz), 1.22-1.30 (1H, m), 3.23 (2H, t, J=6.4 Hz), 3.35-3.53 (6H, m), 3.60 (2H, t, J=6.4 Hz), 3.89 (2H, d, J=6.8 Hz), 6.35 (1H, d, J=2.8 Hz), 7.04 (2H, d, J=8.9 Hz), 7.23 (2H, d, J=8.9 Hz), 7.38 (1H, d, J=2.8 Hz), 12.12 (1H, s).

### Example 143

### 3-[4-(Cyclopropylmethoxy)phenyl]-2-[(2-ethoxyethyl)sulfanyl]-3,5-dihydro-4H-pyrrolo[3,2-d]pyrimidin-4-one

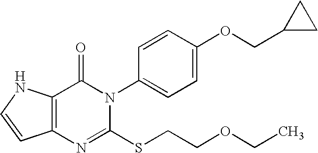

A 1 M aqueous solution of sodium hydrogen carbonate (0.64 ml) was added to a mixture of 3-[4-(cyclopropylmethoxy)phenyl]-2-thioxo-1,2,3,5-tetrahydro-4H-pyrrolo[3,2-d]pyrimidin-4-one (200 mg) obtained by the method of Example 6, or a method pursuant to thereto, 1-chloro-2-ethoxyethane (69 mg), sodium iodide (96 mg) and N,N-dimethylformamide (10 ml), and the resulting mixture was stirred for 15 hours at 100° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (181 mg) was obtained as a white powder.

¹H NMR (300 MHz, DMSO-d₆) δ ppm 0.33-0.39 (2H, m), 0.56-0.65 (2H, m), 1.07(3H, t, J=7.1 Hz), 1.21-1.33 (1H, m), 3.23 (2H, t, J=6.4 Hz), 3.42 (2H, q, J=7.1 Hz), 3.56 (2H, t, J=6.4 Hz), 3.89 (2H, d, J=7.2 Hz), 6.35 (1H, d, J=3.0 Hz), 7.04 (2H, d, J=8.9 Hz), 7.23 (2H, d, J=8.9 Hz), 7.38 (1H, d, J=3.0 Hz), 12.12 (1H, br. s.).

### Example 144

### 3-[4-(Cyclopropylmethoxy)phenyl]-2-[(2-hydroxyethyl)sulfanyl]-3,5-dihydro-4H-pyrrolo[3,2-d]pyrimidin-4-one

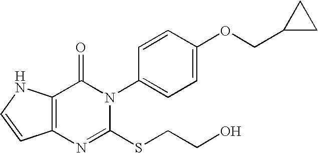

A 1 M aqueous solution of sodium hydrogen carbonate (0.64 ml) was added to a mixture of 3-[4-(cyclopropylmethoxy)phenyl]-2-thioxo-1,2,3,5-tetrahydro-4H-pyrrolo[3,2-d]pyrimidin-4-one (200 mg) obtained by the method of Example 6, or a method pursuant to thereto, 2-bromoethanol (80 mg), sodium iodide (96 mg) and N,N-dimethylformamide (10 ml), and the resulting mixture was stirred for 15 hours at 100° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (110 mg) was obtained as a white powder.

¹H NMR (300 MHz, DMSO-d₆) δ ppm 0.32-0.40 (2H, m), 0.56-0.64 (2H, m), 1.21-1.34 (1H, m), 3.15 (2H, t, J=6.6 Hz), 3.53-3.63 (2H, m), 3.89(2H, d, J=6.8 Hz), 4.91 (1H, t, J=5.5 Hz), 6.34 (1H, d, J=2.7 Hz), 7.04 (2H, d, J=9.1 Hz), 7.23 (2H, d, J=9.1 Hz), 7.38 (1H, d, J=2.7 Hz), 12.10 (1H, s).

### Example 145

### 3-[4-(Cyclopropylmethoxy)phenyl]-2-{[2-(1-methylethoxy)ethyl]sulfanyl}-3,5-dihydro-4H-pyrrolo[3,2-d]pyrimidin-4-one

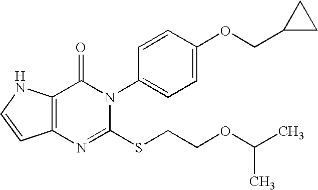

A 1 M aqueous solution of sodium hydrogen carbonate (0.64 ml) was added to a mixture of 3-[4-(cyclopropylmethoxy)phenyl]-2-thioxo-1,2,3,5-tetrahydro-4H-pyrrolo[3,2-d]pyrimidin-4-one (200 mg) obtained by the method of Example 6, or a method pursuant to thereto, 2-(1-methylethoxy)ethyl 4-methylbenzenesulfonate (165 mg) obtained by a method described in a published document, Canadian Journal of Chemistry (Can. J. Chem.), Vol. 33, p. 1207 (1955), or a method pursuant to thereto, sodium iodide (96 mg) and

N,N-dimethylformamide (10 ml), and the resulting mixture was stirred for 3 hours at 100° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (170 mg) was obtained as a white powder.

¹H NMR (300 MHz, DMSO-d₆) δ ppm 0.32-0.39 (2H, m), 0.56-0.64 (2H, m), 1.05 (6 H, d, J=6.1 Hz), 1.20-1.34 (1H, m), 3.19 (2H, t, J=6.4 Hz), 3.51-3.61 (3H, m), 3.89 (2H, d, J=7.2 Hz), 6.34 (1H, d, J=2.5 Hz), 7.04 (2H, d, J=8.5 Hz), 7.23 (2H, d, J=8.5 Hz), 7.38 (1H, d, J=2.5 Hz), 12.11 (1H, br. s.).

### Example 146

### 3-[4-(Cyclopropylmethoxy)-2-fluorophenyl]-2-[(3-ethoxypropyl)sulfanyl]-3,5-dihydro-4H-pyrrolo[3,2-d]pyrimidin-4-one

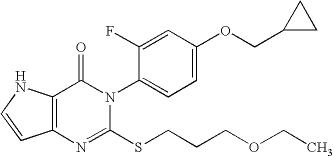

A 1 M aqueous solution of sodium hydrogen carbonate (0.25 ml) was added to a mixture of 3-[4-(cyclopropylmethoxy)-2-fluorophenyl]-2-thioxo-1,2,3,5-tetrahydro-4H-pyrrolo[3,2-d]pyrimidin-4-one (83 mg) obtained by the method of Example 14, or a method pursuant to thereto, 3-ethoxypropyl 4-methylbenzenesulfonate (70 mg) obtained by a method described in a published document, Canadian Journal of Chemistry (Can. J. Chem.), Vol. 33, p. 1207 (1955), or a method pursuant to thereto, sodium iodide (37 mg) and N,N-dimethylformamide (5 ml), and the resulting mixture was stirred for 3 hours at 100° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (55 mg) was obtained as a white powder.

¹H NMR (300 MHz, DMSO-d₆) δ ppm 0.28-0.39 (2H, m), 0.57-0.65 (2H, m), 1.08(3H, t, J=7.0 Hz), 1.21-1.32(1H, m), 1.76-1.89(2H, m), 3.09(2H, t, J=6.8 Hz), 3.34-3.43 (4H, m), 3.92 (2H, d, J=7.2 Hz), 6.37 (1H, d, J=3.0 Hz), 6.91 (1H, dd, J=8.9, 2.4 Hz), 7.06 (1H, dd, J=11.7, 2.4 Hz), 7.33-7.44 (2H, m), 12.19(1H, s).

### Example 147

### 3-[4-(Cyclopropylmethoxy)phenyl]-2-{[2-(tetrahydro-2H-pyran-4-yloxy)ethyl]sulfanyl}-3,5-dihydro-4H-pyrrolo[3,2-d]pyrimidin-4-one

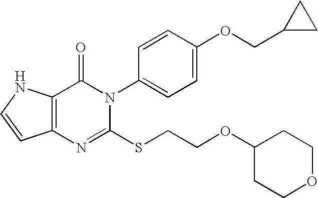

A 1 M aqueous solution of sodium hydrogen carbonate (0.64 ml) was added to a mixture of 3-[4-(cyclopropylmethoxy)phenyl]-2-thioxo-1,2,3, 5-tetrahydro-4H-pyrrolo[3,2-d]pyrimidin-4-one (200 mg) obtained by the method of Example 6, or a method pursuant to thereto, 2-(tetrahydro-2H-pyran-4-yloxy)ethyl 4-methylbenzenesulfonate (192 mg) obtained in Reference Example 55, sodium iodide (96 mg) and N,N-dimethylformamide (5 ml), and the resulting mixture was stirred for 15 hours at 100° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (225 mg) was obtained as a white powder.

¹H NMR (300 MHz, DMSO-d₆) δ ppm 0.33-0.39 (2H, m), 0.57-0.64 (2H, m), 1.20-1.42 (3H, m), 1.75-1.85 (2H, m), 3.22 (2H, t, J=6.5 Hz), 3.25-3.35 (2H, m), 3.46-3.56 (1H, m), 3.62 (2H, t, J=6.5 Hz), 3.77 (2H, dt, J=11.7, 4.2 Hz), 3.89 (2H, d, J=7.0 Hz), 6.33 (1H, d, J=2.8 Hz), 7.04 (2H, d, J=8.9 Hz), 7.22 (2H, d, J=8.9 Hz), 7.38 (1H, d, J=2.8 Hz), 12.10(1H, s).

### Example 148

### 3-[4-(2-Cyclopropylethoxy)phenyl]-2-{[2-(2-ethoxyethoxy)ethyl]sulfanyl}-3,5-dihydro-4H-pyrrolo[3,2-d]pyrimidin-4-one

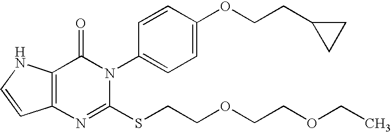

A mixture of 3-[4-(2-cyclopropylethoxy)phenyl]-2-thioxo-1,2,3, 5-tetrahydro-4H-pyrrolo[3,2-d]pyrimidin-4-one (300 mg) obtained in Example 15, 1-bromo-2-(2-ethoxyethoxy)ethane (197 mg), sodium iodide (149 mg), a 1 M aqueous solution of sodium hydrogen carbonate (1 ml) and N,N-dimethylformamide (20 ml) was stirred for 4 hours at 100° C., and then was concentrated under reduced pressure. Ethyl acetate and water were added to the residue, and then the mixture was extracted with ethyl acetate. The organic layer was washed with water and saturated brine, dried over anhydrous sodium sulfate, and then concentrated under reduced pressure. The residue was purified by chromatography, and then was recrystallized from a mixed solvent of ethyl acetate/hexane. Thus, the title compound (311 mg) was obtained as a white solid.

¹H NMR (300 MHz, DMSO-d₆) δ ppm 0.12-0.18 (2H, m), 0.43-0.49 (2H, m), 0.80-0.95 (1H, m), 1.06 (3H, t, J=6.8 Hz), 1.67 (2H, q, J=6.8 Hz), 3.24 (2H, t, J=6.5 Hz), 3.35-3.47 (4H, m), 3.47-3.53 (2H, m), 3.60 (2H, t, J=6.4 Hz), 4.10 (2H, t, J=6.6 Hz), 6.35 (1H, d, J=2.4 Hz), 7.06 (2H, d, J=8.9 Hz), 7.24 (2H, d, J=8.9 Hz), 7.38 (1H, t, J=2.4 Hz), 12.10 (1H, br. s.).

### Example 149

### 3-[4-(2-Cyclopropylethoxy)phenyl]-2-[(2-hydroxyethyl)sulfanyl]-3,5-dihydro-4H-pyrrolo[3,2-d]pyrimidin-4-one

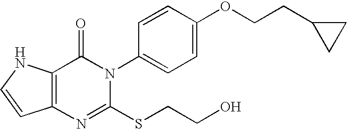

A mixture of 3-[4-(2-cyclopropylethoxy)phenyl]-2-thioxo-1,2,3,5-tetrahydro-4H-pyrrolo[3,2-d]pyrimidin-4-one (300 mg) obtained in Example 15, 2-bromoethanol (140 μl), sodium iodide (149 mg), a 1 M aqueous solution of sodium hydrogen carbonate (1.5 ml) and

N,N-dimethylformamide (20 ml) was stirred overnight at 100° C., and then was concentrated under reduced pressure. Ethyl acetate and water were added to the residue, and the mixture was extracted with ethyl acetate. The organic layer was washed with water and saturated brine, dried over anhydrous sodium sulfate, and then concentrated under reduced pressure. The residue was purified by chromatography, and then was recrystallized from a mixed solvent of ethyl acetate/hexane. Thus, the title compound (161 mg) was obtained as a white solid.

¹H NMR (300 MHz, DMSO-d₆) δ ppm 0.13-0.18 (2H, m), 0.43-0.50 (2H, m), 0.80-0.95 (1H, m), 1.67 (2H, q, J=6.8 Hz), 3.15 (2H, t, J=6.5 Hz), 3.59 (2H, q, J=6.4 Hz), 4.10 (2H, t, J=6.5 Hz), 4.89 (1H, t, J=5.4 Hz), 6.34 (1H, d, J=2.8 Hz), 7.06 (2H, d, J=8.9 Hz), 7.24 (2H, d, J=8.9 Hz), 7.38 (1H, br. s.), 12.09 (1H, br. s.).

### Example 150

### N-(2-cyanoethyl)-4-({3-[4-(2-cyclopropylethoxy)phenyl]-4-oxo-4,5-dihydro-3H-pyrrolo[3,2-d]pyrimidin-2-yl}sulfanyl)butanamide

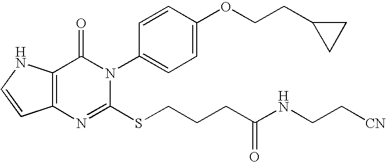

A mixture of 3-[4-(2-cyclopropylethoxy)phenyl]-2-thioxo-1,2,3,5-tetrahydro-4H-pyrrolo[3,2-d]pyrimidin-4-one (300 mg) obtained in Example 15, 4-bromo-N-(2-cyanoethyl)butanamide (306 mg) obtained in Reference Example 49, sodium iodide (225 mg), triethylamine (278 μl) and N,N-dimethylformamide (20 ml) was stirred overnight at 90° C., and then was concentrated under reduced pressure. Ethyl acetate and water were added to the residue, and then the mixture was extracted with ethyl acetate. The organic layer was washed with water and saturated brine, dried over anhydrous sodium sulfate, and then concentrated under reduced pressure. The residue was purified by chromatography, and then was recrystallized from a mixed solvent of ethyl acetate/hexane. Thus, the title compound (234 mg) was obtained as a white solid.

¹H NMR (300 MHz, DMSO-d₆) δ ppm 0.12-0.19 (2H, m), 0.42-0.50 (2H, m), 0.80-0.95 (1H, m), 1.67 (2H, q, J=6.6 Hz), 1.75-1.89 (2H, m), 2.17 (2H, t, J=7.4 Hz), 2.60 (2H, t, J=6.5 Hz), 3.05 (2H, t, J=7.2 Hz), 3.25 (2H, q, J=6.3 Hz), 4.10(2H, t, J=6.5 Hz), 6.34 (1H, dd, J=2.6, 2.1 Hz), 7.06 (2H, d, J=8.9 Hz), 7.24 (2H, d, J=8.9 Hz), 7.37 (1H, t, J=2.9 Hz), 8.19 (1H, t, J=5.7 Hz), 12.09 (1H, br. s.).

### Example 151

### 2-{[3-(Methylsulfonyl)propyl]sulfanyl}-3-{4-[(2,2,2-trifluoroethoxy)methyl]phenyl}-3,5-dihydro-4H-pyrrolo[3,2-d]pyrimidin-4-one

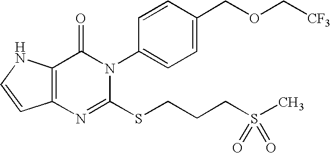

A mixture of 2-thioxo-3-{4-[(2,2,2-trifluoroethoxy)methyl]phenyl}-1,2,3,5-tetrahydro-4H-pyrrolo[3,2-d]pyrimidin-4-one (250 mg) obtained in Example 16, 3-(methylsulfonyl)propyl 4-methylbenzenesulfonate (225 mg) obtained by the method described in a published document, WO 08/1931, or a method pursuant to thereto, sodium iodide (149 mg), a 1 M aqueous solution of sodium hydrogen carbonate (1.0 ml) and N,N-dimethylformamide (10 ml), was stirred overnight at 100° C., and then was concentrated under reduced pressure. Ethyl acetate and water were added to the residue, and the mixture was extracted with ethyl acetate. The organic layer was washed with water and saturated brine, dried over anhydrous sodium sulfate, and then concentrated under reduced pressure. The residue was purified by chromatography, and then was recrystallized from ethyl acetate. Thus, the title compound (187 mg) was obtained as a white solid.

¹H NMR (300 MHz, DMSO-d₆) δ ppm 2.00-2.12 (2H, m), 2.97 (3H, s), 3.13-3.21 (4H, m), 4.20 (2H, q, J=9.5 Hz), 4.77 (2H, s), 6.36 (1H, d, J=2.3 Hz), 7.37-7.43 (3H, m), 7.51 (2H, d, J=8.3 Hz), 12.17(1H, br. s.).

### Example 152

### 2-{[2-(2-Ethoxyethoxy)ethyl]sulfanyl}-3-{4-[(2,2,2-trifluoroethoxy)methyl]phenyl}-3,5-dihydro-4H-pyrrolo[3,2-d]pyrimidin-4-one

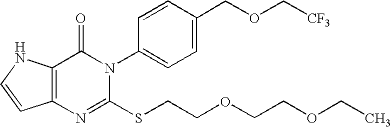

A mixture of 2-thioxo-3-{4-[(2,2,2-trifluoroethoxy)methyl]phenyl}-1,2,3,5-tetrahydro-4H-pyrrolo[3,2-d]pyrimidin-4-one (250 mg) obtained in Example 16, 1-bromo-2-(2-ethoxyethoxy)ethane (197 mg), sodium iodide (149 mg), a 1 M aqueous solution of sodium hydrogen carbonate (840 μl) and N,N-dimethylformamide (10 ml) was stirred for 4 hours at 90° C., cooled to room temperature, and then concentrated under reduced pressure. Ethyl acetate and water were added to the residue, and then the mixture was extracted with ethyl acetate. The organic layer was washed with water and saturated brine, dried over anhydrous sodium sulfate, and then concentrated under reduced pressure. The residue was purified by chromatography, and then was recrystallized from diisopropyl ether, and thus the title compound (187 mg) was obtained as a white solid.

¹H NMR (300 MHz, DMSO-d₆) δ ppm 1.06 (3H, t, J=7.0 Hz), 3.25 (2H, t, J=6.4 Hz), 3.34-3.46 (4H, m), 3.47-3.53 (2H, m), 3.60 (2H, t, J=6.4 Hz), 4.20 (2H, q, J=9.2 Hz), 4.77 (2H, s), 6.37 (1H, d, J=2.7 Hz), 7.35-7.40 (3H, m), 7.51 (2H, d, J=8.3 Hz), 12.15 (1H, br. s.).

### Example 153

### 3-[4-(Cyclopropylmethoxy)phenyl]-2-(ethylsulfanyl)-3,5-dihydro-4H-pyrrolo[3,2-d]pyrimidin-4-one

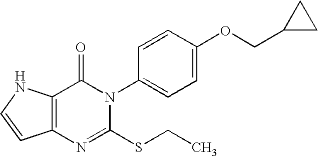

A 1 M aqueous solution of sodium hydrogen carbonate (0.64 ml) was added to a mixture of 3-[4-(cyclopropylmethoxy)phenyl]-2-thioxo-1,2,3,5-tetrahydro-4H-pyrrolo[3,2-d]pyrimidin-4-one (200 mg) obtained by the method of Example 6, or a method pursuant to thereto, iodoethane (204 μl) and N,N-dimethylformamide (10 ml), and the resulting mixture was stirred for 15 hours at 70° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (186 mg) was obtained as a white powder.

¹H NMR (300 MHz, DMSO-d₆) δ ppm 0.33-0.39 (2H, m), 0.56-0.64 (2H, m), 1.16-1.34 (4H, m), 3.02 (2H, q, J=7.2 Hz), 3.89 (2H, d, J=7.2 Hz), 6.35 (1H, d, J=3.0 Hz), 7.03 (2H, d, J=8.9 Hz), 7.22 (2H, d, J=8.9 Hz), 7.37 (1H, d, J=3.0 Hz), 12.09 (1H, s).

### Example 154

### 3-[4-(Cyclopropylmethoxy)phenyl]-2-{[3-(methylsulfonyl)propyl]sulfanyl}-3,5-dihydro-4H-pyrrolo[3,2-d]pyrimidin-4-one

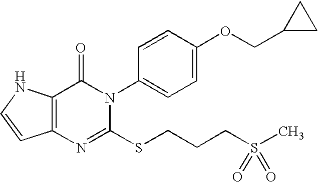

A 1 M aqueous solution of sodium hydrogen carbonate (0.64 ml) was added to a mixture of 3-[4-(cyclopropylmethoxy)phenyl]-2-thioxo-1,2,3,5-tetrahydro-4H-pyrrolo[3,2-d]pyrimidin-4-one (200 mg) obtained by the method of Example 6, or a method pursuant to thereto, 3-(methylsulfonyl)propyl 4-methylbenzenesulfonate (187 mg) obtained by a method described in a published document, WO 08/1931, or a method pursuant to thereto, sodium iodide (96 mg) and N,N-dimethylformamide (10 ml), and the resulting mixture was stirred for 15 hours at 100° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (164 mg) was obtained as a white powder.

¹H NMR (300 MHz, DMSO-d₆) δ ppm 0.33-0.39 (2H, m), 0.57-0.64 (2H, m), 1.19-1.34 (1H, m), 2.00-2.12 (2H, m), 2.97 (3H, s), 3.10-3.23 (4H, m), 3.89 (2H, d, J=6.8 Hz), 6.34 (1H, d, J=2.8 Hz), 7.04 (2H, d, J=9.1 Hz), 7.25 (2H, d, J=9.1 Hz), 7.39 (1H, d, J=2.8 Hz), 12.12(1H, s).

### Example 155

### 3-[4-(Cyclopropylmethoxy)phenyl]-2-{[2-(4-hydroxytetrahydro-2H-pyran-4-yl)ethyl]sulfanyl}-3,5-dihydro-4H-pyrrolo[3,2-d]pyrimidin-4-one

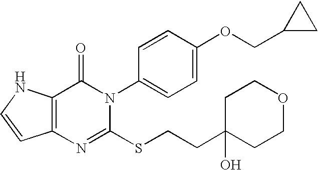

A 1 M aqueous solution of sodium hydrogen carbonate (0.64 ml) was added to a mixture of 3-[4-(cyclopropylmethoxy)phenyl]-2-thioxo-1,2,3,5-tetrahydro-4H-pyrrolo[3,2-d]pyrimidin-4-one (200 mg) obtained by the method of Example 6, or a method pursuant to thereto, 2-(4-hydroxytetrahydro-2H-pyran-4-yl)ethyl 4-methylbenzenesulfonate (192 mg) obtained in Reference Example 56, sodium iodide (96 mg) and N,N-dimethylformamide (5 ml), and the resulting mixture was stirred for 15 hours at 100° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (165 mg) was obtained as a white powder.

¹H NMR (300 MHz, DMSO-d₆) δ ppm 0.31-0.39 (2H, m), 0.56-0.64 (2H, m), 1.20-1.79 (7H, m), 3.00-3.12 (2H, m), 3.48-3.69 (4H, m), 3.88 (2H, d, J=7.2 Hz), 4.41 (1H, br. s.), 6.32 (1H, d, J=2.5 Hz), 7.03 (2H, d, J=8.9 Hz), 7.22 (2H, d, J=8.9 Hz), 7.37 (1H, d, J=2.5 Hz), 12.09 (1H, br. s.).

### Example 156

### 3-[4-(Cyclopropylmethoxy)phenyl]-2-[(3-ethoxy-2-hydroxypropyl)sulfanyl]-3,5-dihydro-4H-pyrrolo[3,2-d]pyrimidin-4-one

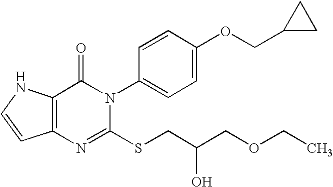

A 1 M aqueous solution of sodium hydrogen carbonate (0.64 ml) was added to a mixture of 3-[4-(cyclopropylmethoxy)phenyl]-2-thioxo-1,2,3,5-tetrahydro-4H-pyrrolo[3,2-d]pyrimidin-4-one (200 mg) obtained by the method of Example 6, or a method pursuant to thereto, 2-(ethoxymethyl)oxirane (65 mg), sodium iodide (96 mg) and N,N-dimethylformamide (5 ml), and the resulting mixture was stirred for 15 hours at 100° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (100 mg) was obtained as a white powder.

¹H NMR (300 MHz, DMSO-d₆) δ ppm 0.32-0.40 (2H, m), 0.56-0.65 (2H, m), 1.09(3H, t, J=7.0 Hz), 1.21-1.33 (1H, m), 3.02 (1H, dd, J=13.1, 7.8 Hz), 3.24-3.34 (3H, m), 3.38-3.48 (2H, m), 3.72-3.84 (1H, m), 3.89 (2H, d, J=7.2 Hz), 5.09 (1H, d, J=5.7 Hz), 6.33 (1H, d, J=3.0 Hz), 7.04 (2H, d, J=8.9 Hz), 7.23 (2H, d, J=8.9 Hz), 7.37 (1H, d, J=3.0 Hz), 12.09 (1H, s).

### Example 157

### 3-[4-(Cyclopropylmethoxy)phenyl]-2-{[3-(tetrahydro-2H-pyran-4-yloxy)propyl]sulfanyl}-3,5-dihydro-4H-pyrrolo[3,2-d]pyrimidin-4-one

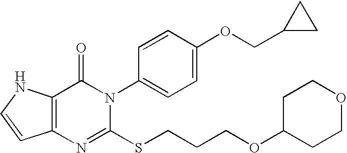

A 1 M aqueous solution of sodium hydrogen carbonate (0.59 ml) was added to a mixture of 3-[4-(cyclopropylmethoxy)phenyl]-2-thioxo-1,2,3,5-tetrahydro-4H-pyrrolo[3,2-d]pyrimidin-4-one (200 mg) obtained by the method of Example 6, or a method pursuant to thereto, 2-(4-hydroxytetrahydro-2H-pyran-4-yl)ethyl 4-methylbenzenesulfonate (185 mg) obtained in Reference Example 59, sodium iodide (88 mg) and N,N-dimethylformamide (7 ml), and the resulting mixture was stirred for 15 hours at 100° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (235 mg) was obtained as a white powder.

¹H NMR (300 MHz, DMSO-d₆) δ ppm 0.32-0.39 (2H, m), 0.55-0.65 (2H, m), 1.22-1.41 (3H, m), 1.72-1.87 (4H, m), 3.08 (2H, t, J=7.2 Hz), 3.24-3.35 (2H, m), 3.37-3.49 (3H, m), 3.69-3.82 (2H, m), 3.88 (2H, d, J=6.8 Hz), 6.33 (1H, d, J=2.5 Hz), 7.03 (2H, d, J=8.7 Hz), 7.22 (2H, d, J=8.7 Hz), 7.37 (1H, d, J=2.5 Hz), 12.09 (1H, br. s.).

### Example 158

### 2-{[3-(Tetrahydro-2H-pyran-4-yloxy)propyl]sulfanyl}-3-[4-(2,2,2-trifluoroethoxy)phenyl]-3,5-dihydro-4H-pyrrolo[3,2-d]pyrimidin-4-one

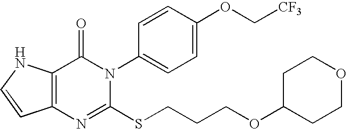

A 1 M aqueous solution of sodium hydrogen carbonate (0.59 ml) was added to a mixture of 2-thioxo-3-[4-(2,2,2-trifluoroethoxy)phenyl]-1,2,3,5-tetrahydro-4H-pyrrolo[3,2-d]pyrimidin-4-one (200 mg) obtained by the method of Example 2, or a method pursuant to thereto, 2-(4-hydroxytetrahydro-2H-pyran-4-yl)ethyl 4-methylbenzenesulfonate (184 mg) obtained in Reference Example 59, sodium iodide (88 mg) and N,N-dimethylformamide (10 ml), and the resulting mixture was stirred for 15 hours at 100° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (251 mg) was obtained as a white powder.

¹H NMR (300 MHz, DMSO-d₆) δ ppm 1.27-1.41 (2H, m), 1.75-1.87 (4H, m), 3.09 (2H, t, J=7.0 Hz), 3.25-3.35 (2H, m), 3.36-3.48 (3H, m), 3.71-3.81 (2H, m), 4.87 (2H, q, J=8.7 Hz), 6.34 (1H, d, J=2.7 Hz), 7.19 (2H, d, J=9.0 Hz), 7.33 (2H, d, J=9.0 Hz), 7.38 (1H, d, J=2.7 Hz), 12.11 (1H, s).

### Example 159

### 2-{[2-(Tetrahydro-2H-pyran-4-yloxy)ethyl]sulfanyl}-3-[4-(2,2,2-trifluoroethoxy)phenyl]-3,5-dihydro-4H-pyrrolo[3,2-d]pyrimidin-4-one

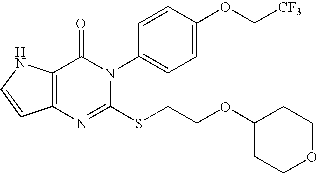

A 1 M aqueous solution of sodium hydrogen carbonate (0.59 ml) was added to a mixture of 2-thioxo-3-[4-(2,2,2-trifluoroethoxy)phenyl]-1,2,3,5-tetrahydro-4H-pyrrolo[3,2-d]pyrimidin-4-one (200 mg) obtained by the method of Example 2, or a method pursuant to thereto, 2-(tetrahydro-2H-pyran-4-yloxy)ethyl 4-methylbenzenesulfonate (177 mg) obtained in Reference Example 55, sodium iodide (88 mg) and N,N-dimethylformamide (5 ml), and the resulting mixture was stirred for 15 hours at 100° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (181 mg) was obtained as a white powder.

¹H NMR (300 MHz, DMSO-d₆) δ ppm 1.28-1.42 (2H, m), 1.75-1.86 (2H, m), 3.19-3.28 (2H, m), 3.28-3.35 (2H, m), 3.51 (1H, ddd, J=8.9, 4.7, 4.5 Hz), 3.62 (2H, t, J=6.6 Hz), 3.77 (2H, dt, J=11.6, 4.2 Hz), 4.87 (2H, q, J=9.1 Hz), 6.34 (1H, d, J=2.7 Hz), 7.19 (2H, d, J=9.0 Hz), 7.33 (2H, d, J=9.0 Hz), 7.39 (1H, d, J=2.7 Hz), 12.13 (1H, s).

### Example 160

### 3-[4-(Cyclopropylmethoxy)-3-methylphenyl]-2-(ethylsulfanyl)-3,5-dihydro-4H-pyrrolo[3,2-d]pyrimidin-4-one

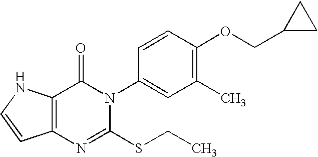

A mixture of 3-[4-(cyclopropylmethoxy)-3-methylphenyl]-2-thioxo-1,2,3,5-tetrahydro-4H-pyrrolo[3,2-d]pyrimidin-4-one (230 mg) obtained in Example 17, iodoethane (640 μl), a 1 M aqueous solution of sodium hydrogen carbonate (840 μl) and N,N-dimethylformamide (20 ml) was stirred for 4 hours at 90° C., cooled to room temperature, and then concentrated under reduced pressure. Water was added to the residue, and then the mixture was extracted with ethyl acetate. The organic layer was washed with water and saturated brine, dried over anhydrous sodium sulfate, and then concentrated under reduced pressure. The residue was recrystallized from ethyl acetate, and thus the title compound (151 mg) was obtained as a white solid.

¹H NMR (300 MHz, DMSO-d₆) δ ppm 0.34-0.41 (2H, m), 0.57-0.64 (2H, m), 1.18-1.34 (4H, m), 2.20 (3H, s), 3.01 (2H, q, J=7.2 Hz), 3.91 (2H, d, J=6.8 Hz), 6.33-6.35 (1H, m), 6.96-7.04 (1H, m), 7.04-7.12 (2H, m), 7.37 (1H, t, J=2.8 Hz), 12.08 (1H, br. s.).

### Example 161

### 3-[4-(Cyclopropylmethoxy)-3-methylphenyl]-2-{[2-(2-ethoxyethoxy)ethyl]sulfanyl}-3,5-dihydro-4H-pyrrolo[3,2-d]pyrimidin-4-one

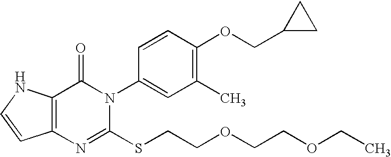

A mixture of 3-[4-(cyclopropylmethoxy)-3-methylphenyl]-2-thioxo-1,2,3,5-tetrahydro-4H-pyrrolo[3,2-d]pyrimidin-4-one (230 mg) obtained in Example 17, 1-bromo-2-(2-ethoxyethoxy)ethane (197 mg), sodium iodide (149 mg), a 1 M aqueous solution of sodium hydrogen carbonate (840 μl) and N,N-dimethylformamide (20 ml) was stirred for 4 hours at 90° C., cooled to room temperature, and then concentrated under reduced pressure. Water was added to the residue, and then the mixture was extracted with ethyl acetate. The organic layer was washed with water and saturated brine, dried over anhydrous sodium sulfate, and then concentrated under reduced pressure. The residue was purified by chromatography, and then was recrystallized from a mixed solvent of ethyl acetate/diisopropyl ether. Thus, the title compound (182 mg) was obtained as a white solid.

¹H NMR (300 MHz, DMSO-d₆) δ ppm 0.34-0.41 (2H, m), 0.56-0.64 (2H, m), 1.06 (3H, t, J=7.0 Hz), 1.20-1.36 (1H, m), 2.20 (3H, s), 3.23 (2H, t, J=6.4 Hz), 3.34-3.47 (4H, m), 3.47-3.53 (2H, m), 3.60 (2H, t, J=6.6 Hz), 3.84-3.98 (2H, m), 6.34 (1H, d, J=3.0 Hz), 6.96-7.05 (1H, m), 7.05-7.13 (2H, m), 7.37 (1H, d, J=2.7 Hz), 12.09 (1H, s).

### Example 162

### 3-[3-Chloro-4-(cyclopropylmethoxy)phenyl]-2-(ethylsulfanyl)-3,5-dihydro-4H-pyrrolo[3,2-d]pyrimidin-4-one

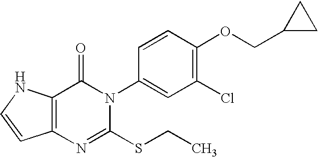

A mixture of 3-[3-chloro-4-(cyclopropylmethoxy)phenyl]-2-thioxo-1,2,3,5-tetrahydro-4H-pyrrolo[3,2-d]pyrimidin-4-one (243 mg) obtained in Example 18, iodoethane (640 μl), a 1 M aqueous solution of sodium hydrogen carbonate (840 μl) and N,N-dimethylformamide (20 ml) was stirred for 4 hours at 90° C., cooled to room temperature, and then concentrated under reduced pressure. Water was added to the residue, and then the mixture was extracted with ethyl acetate. The organic layer was washed with water and saturated brine, dried over anhydrous sodium sulfate, and then concentrated under reduced pressure. The residue was recrystallized from a mixed solvent of ethyl acetate/diisopropyl ether, and thus the title compound (150 mg) was obtained as a white solid.

¹H NMR (300 MHz, DMSO-d₆) δ ppm 0.36-0.42 (2H, m), 0.59-0.66 (2H, m), 1.20-1.37 (4H, m), 3.04 (2H, q, J=7.4 Hz), 3.93-4.08 (2H, m), 6.35 (1H, t, J=2.3 Hz), 7.23 (1H, d, J=9.1 Hz), 7.29 (1H, dd, J=9.1, 2.3 Hz), 7.38 (1H, t, J=2.8 Hz), 7.52 (1H, d, J=2.3 Hz), 12.12 (1H, br. s.).

### Example 163

### 3-[3-Chloro-4-(cyclopropylmethoxy)phenyl]-2-{[2-(2-ethoxyethoxy)ethyl]sulfanyl}-3,5-dihydro-4H-pyrrolo[3,2-d]pyrimidin-4-one

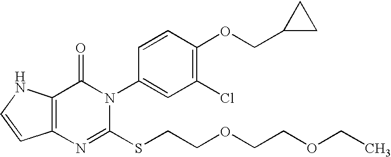

A mixture of 3-[3-chloro-4-(cyclopropylmethoxy)phenyl]-2-thioxo-1,2,3,5-tetrahydro-4H-pyrrolo[3,2-d]pyrimidin-4-one (243 mg) obtained in Example 18, 1-bromo-2-(2-ethoxyethoxy)ethane (197 mg), sodium iodide (149 mg), a 1 M aqueous solution of sodium hydrogen carbonate (840 μl) and N,N-dimethylformamide (20 ml) was stirred for 4 hours at 90° C., cooled to room temperature, and then concentrated under reduced pressure. Water was added to the residue, and then the mixture was extracted with ethyl acetate. The organic layer was washed with water and saturated brine, dried over anhydrous sodium sulfate, and then concentrated under reduced pressure. The residue was purified by chromatography, and then was recrystallized from a mixed solvent of ethyl acetate/diisopropyl ether. Thus, the title compound (158 mg) was obtained as a white solid.

¹H NMR (300 MHz, DMSO-d₆) δ ppm 0.36-0.42 (2H, m), 0.59-0.66 (2H, m), 1.06(3H, t, J=7.0 Hz), 1.23-1.38 (1H, m), 3.25 (2H, t, J=6.4 Hz), 3.35-3.46 (4H, m), 3.48-3.53 (2H, m), 3.61 (2H, t, J=6.4 Hz), 3.95-4.07 (2H, m), 6.35 (1H, d, J=2.7 Hz), 7.24 (1H, d, J=8.7 Hz), 7.30 (1H, dd, J=8.7, 2.3 Hz), 7.39 (1H, d, J=2.7 Hz), 7.53 (1H, d, J=2.3 Hz), 12.14 (1H, br. s.).

### Example 164

### 3-[4-(Cyclopropylmethoxy)phenyl]-2-({3-[(1-methylethyl)sulfonyl]propyl}sulfanyl)-3,5-dihydro-4H-pyrrolo[3,2-d]pyrimidin-4-one

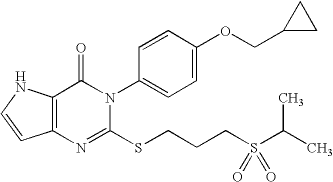

A 1 M aqueous solution of sodium hydrogen carbonate (0.64 ml) was added to a mixture of 3-[4-(cyclopropylmethoxy)phenyl]-2-thioxo-1,2,3,5-tetrahydro-4H-pyrrolo[3,2-d]pyrimidin-4-one (200 mg) obtained by the method of Example 6, or a method pursuant to thereto, 3-[(1-methylethyl)sulfonyl]propyl 4-methylbenzenesulfonate (205 mg) obtained in

Reference Example 62, sodium iodide (96 mg) and N,N-dimethylformamide (10 ml), and the resulting mixture was stirred for 15 hours at 100° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (160 mg) was obtained as a white powder.

¹H NMR (300 MHz, DMSO-d₆) δ ppm 0.32-0.39 (2H, m), 0.56-0.64 (2H, m), 1.19-1.32(7 H, m), 1.98-2.12 (2H, m), 3.11-3.19 (4H, m), 3.22-3.31 (1H, m), 3.89 (2H, d, J=7.2 Hz), 6.33 (1H, d, J=2.8 Hz), 7.04 (2H, d, J=8.9 Hz), 7.25 (2H, d, J=8.9 Hz), 7.39 (1H, d, J=2.8 Hz), 12.12 (1H, s).

### Example 165

### 2-({3-[(Cyclopropylmethyl)sulfonyl]propyl}sulfanyl)-3-[4-(2,2,2-trifluoroethoxy)phenyl]-3,5-dihydro-4H-pyrrolo[3,2-d]pyrimidin-4-one

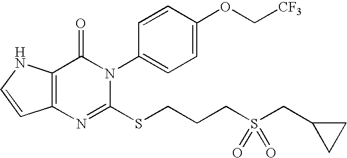

A 1 M aqueous solution of sodium hydrogen carbonate (0.59 ml) was added to a mixture of 2-thioxo-3-[4-(2,2,2-trifluoroethoxy)phenyl]-1,2,3,5-tetrahydro-4H-pyrrolo[3,2-d]pyrimidin-4-one (200 mg) obtained by the method of Example 2, or a method pursuant to thereto, 3-[(cyclopropylmethyl)sulfonyl]propyl 4-methylbenzenesulfonate (196 mg) obtained in Reference Example 65, sodium iodide (88 mg) and N,N-dimethylformamide (10 ml), and the resulting mixture was stirred for 15 hours at 100° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (150 mg) was obtained as a white powder.

¹H NMR (300 MHz, DMSO-d₆) δ ppm 0.29-0.37 (2H, m), 0.54-0.63 (2H, m), 0.92-1.09 (1H, m), 2.00-2.12 (2H, m), 3.05 (2H, d, J=7.2 Hz), 3.10-3.24 (4H, m), 4.87 (2H, q, J=9.0 Hz), 6.35 (1H, dd, J=2.8, 2.1 Hz), 7.20 (2H, d, J=8.9 Hz), 7.35 (2H, d, J=8.9 Hz), 7.40 (1H, t, J=2.9 Hz), 12.14 (1H, br. s.).

### Example 166

### 3-[4-(Cyclopropylmethoxy)phenyl]-2-({3-[(cyclopropylmethyl)sulfonyl]propyl}sulfanyl)-3,5-dihydro-4H-pyrrolo[3,2-d]pyrimidin-4-one

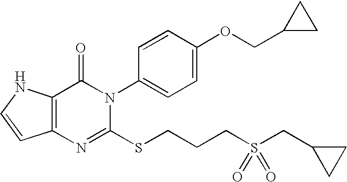

A 1 M aqueous solution of sodium hydrogen carbonate (0.64 ml) was added to a mixture of 3-[4-(cyclopropylmethoxy)phenyl]-2-thioxo-1,2,3,5-tetrahydro-4H-pyrrolo[3,2-d]pyrimidin-4-one (200 mg) obtained by the method of Example 6, or a method pursuant to thereto, 3-[(cyclopropylmethyl)sulfonyl]propyl 4-methylbenzenesulfonate (212 mg) obtained in Reference Example 65, sodium iodide (96 mg) and N,N-dimethylformamide (10 ml), and the resulting mixture was stirred for 15 hours at 100° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (210 mg) was obtained as a white powder.

¹H NMR (300 MHz, DMSO-d₆) δ ppm 0.29-0.39 (4H, m), 0.55-0.64 (4H, m), 0.93-1.08 (1H, m), 1.21-1.34 (1H, m), 1.99-2.11 (2H, m), 3.05 (2H, d, J=7.2 Hz), 3.12-3.19 (4H, m), 3.89 (2H, d, J=7.0 Hz), 6.34 (1H, d, J=2.6 Hz), 7.04 (2H, d, J=8.9 Hz), 7.25 (2H, d, J=8.9 Hz), 7.39 (1H, br. s.), 12.12 (1H, br. s.).

### Example 167

### 2-{[2-Hydroxy-3-(tetrahydro-2H-pyran-4-yloxy)propyl]sulfanyl}-3-[4-(2,2,2-trifluoroethoxy)phenyl]-3,5-dihydro-4H-pyrrolo[3,2-d]pyrimidin-4-one

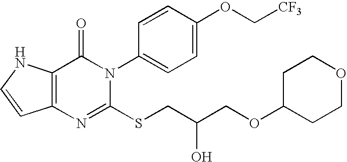

A 1 M aqueous solution of sodium hydrogen carbonate (0.59 ml) was added to a mixture of 2-thioxo-3-[4-(2,2,2-trifluoroethoxy)phenyl]-1,2,3,5-tetrahydro-4H-pyrrolo[3,2-d]pyrimidin-4-one (200 mg) obtained by the method of Example 2, or a method pursuant to thereto, 4-(oxiran-2-ylmethoxy)tetrahydro-2H-pyrane (93 mg) obtained in Reference Example 66, sodium iodide (88 mg) and N,N-dimethylformamide (10 ml), and the resulting mixture was stirred for 15 hours at 100° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (135 mg) was obtained as a white powder.

¹H NMR (300 MHz, DMSO-d₆) δ ppm 1.27-1.45 (2H, m), 1.70-1.86 (2H, m), 3.08 (1H, dd, J=13.1, 7.4 Hz), 3.23-3.52 (7 H, m), 3.71-3.84 (2H, m), 4.87 (2H, q, J=8.9 Hz), 6.28-6.35 (1H, m), 7.19 (2H, d, J=8.9 Hz), 7.33 (2H, d, J=8.9 Hz), 7.36-7.42 (1H, m), 12.10(1H, br. s.).

### Example 168

### N-(2-cyanoethyl)-4-({6-methyl-4-oxo-3-[4-(2,2,2-trifluoroethoxy)phenyl]-4,5-dihydro-3H-pyrrolo[3,2-d]pyrimidin-2-yl}sulfanyl)butanamide

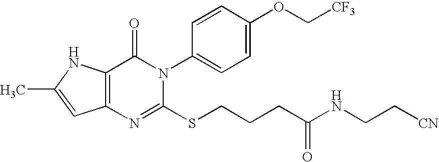

A mixture of 6-methyl-2-thioxo-3-[4-(2,2,2-trifluoroethoxy)phenyl]-1,2,3,5-tetrahydro-4H-pyrrolo[3,2-d]pyrimidin-4-one (178 mg) obtained by the method of Example 19, or a method pursuant to thereto, 4-bromo-N-(2-cyanoethyl)butanamide (131 mg) obtained by the method of Reference Example 49, or a method pursuant to thereto, sodium iodide (75 mg), triethylamine (140 μl) and N,N-dimethylformamide (5 ml) was heated to 100° C., and was stirred for 12 hours. The reaction mixture was returned to room temperature, and then was diluted with ethyl acetate (80 ml). The dilution was washed with water and saturated brine, dried over anhydrous magnesium sulfate, and then concentrated under reduced pressure. The resulting residue was purified by chromatography, and thus the title compound (156 mg) was obtained as a white solid.

¹H NMR (300 MHz, DMSO-d₆) δ ppm 1.81 (2H, tt, J=7.4, 7.1 Hz), 2.16 (2H, t, J=7.4 Hz), 2.32 (3H, s), 2.60 (2H, t, J=6.5 Hz), 3.04 (2H, t, J=7.1 Hz), 3.24 (2H, td, J=6.5, 5.7 Hz), 4.87 (2H, q, J=8.9 Hz), 6.08 (1H, s), 7.18 (2H, d, J=9.0 Hz), 7.31 (2H, d, J=9.0 Hz), 8.20 (1H, t, J=5.7 Hz), 11.88 (1H, s).

### Example 169

### 2-(Ethylsulfanyl)-3-[4-(2,2,2-trifluoroethoxy)phenyl]-5,7-dihydro-3H-pyrrolo[3,2-d]pyrimidine-4,6-dione and its tautomer

### 2-(ethylsulfanyl)-6-hydroxy-3-[4-(2,2,2-trifluoroethoxy)phenyl]-3,5-dihydro-4H-pyrrolo[3,2-d]pyrimidin-4-one

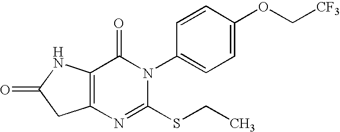

A mixture of 2-thioxo-3-[4-(2,2,2-trifluoroethoxy)phenyl]-2,3,4a,5-tetrahydro-1H-pyrrolo[3,2-d]pyrimidine-4,6-dione (68 mg) obtained by the method of Example 20, or a method pursuant to thereto, a 1 M aqueous solution of sodium hydrogen carbonate (190 μl), iodoethane (297 mg, 154 μl) and acetonitrile (4 ml) was heated to reflux for 30 minutes. The reaction mixture solution was returned to room temperature, and then was diluted with ethyl acetate (50 ml). The dilution was washed with water and saturated brine, dried over anhydrous magnesium sulfate, and concentrated under reduced pressure. A crude product obtained therefrom was purified by chromatography, and thus a tautomeric mixture of the title compound (48 mg) was obtained as a white solid.

¹H NMR (300 MHz, DMSO-d₆) δ ppm 1.21 (3H, t, J=7.4 Hz), 3.00 (2H, q, J=7.4 Hz), 3.62 (1.2H, s), 4.86 (2H, q, J=8.9 Hz), 5.32 (0.4H, br. s.), 7.00-7.53 (4H, m), 10.47 (0.6 H, s), 11.21 (0.4H, br. s.), 11.46 (0.4H, br. s.).

### Example 170

### N-(2-cyanoethyl)-4-({7-cyclopropyl-4-oxo-3-[4-(2,2,2-trifluoroethoxy)phenyl]-4,5-dihydro-3H-pyrrolo[3,2-d]pyrimidin-2-yl}sulfanyl)butanamide

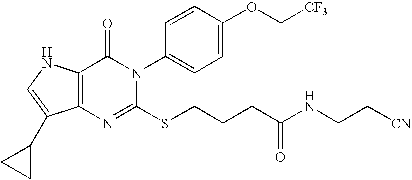

A mixture of 7-cyclopropyl-2-thioxo-3-[4-(2,2,2-trifluoroethoxy)phenyl]-1,2,3,5-tetrahydro-4H-pyrrolo[3,2-d]pyrimidin-4-one (191 mg) obtained by the method of Example 21, or a method pursuant to thereto, 4-bromo-N-(2-cyanoethyl)butanamide (131 mg) obtained by the method of Reference Example 49, or a method pursuant to thereto, sodium iodide (75 mg), triethylamine (140 μl) and N,N-dimethylformamide (5 ml) was heated to 100° C., and was stirred for 12 hours. The reaction mixture was returned to room temperature, and then was diluted with ethyl acetate (80 ml). The dilution was washed with water and saturated brine, dried over anhydrous magnesium sulfate, and then concentrated under reduced pressure. The resulting residue was purified by chromatography, and thus the title compound (221 mg) was obtained as a yellowish white solid.

¹H NMR (300 MHz, DMSO-d₆) δ ppm 0.79-0.89 (2H, m), 0.90-0.98 (2H, m), 1.78-1.97 (3H, m), 2.19 (2H, t, J=7.5 Hz), 2.61 (2H, t, J=6.4 Hz), 3.04 (2H, t, J=7.2 Hz), 3.25 (2H, td, J=6.4, 5.7 Hz), 4.87 (2H, q, J=8.8 Hz), 7.15-7.21 (3H, m), 7.31 (2H, d, J=9.1 Hz), 8.21 (1H, t, J=5.7 Hz), 11.78 (1H, br. s.).

### Example 171

### N-(2-cyanoethyl)-4-({7-ethyl-4-oxo-3-[4-(2,2,2-trifluoroethoxy)phenyl]-4,5-dihydro-3H-pyrrolo[3,2-d]pyrimidin-2-yl}sulfanyl)butanamide

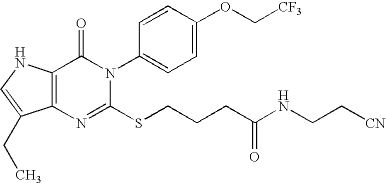

A mixture of 7-ethyl-2-thioxo-3-[4-(2,2,2-trifluoroethoxy)phenyl]-1,2,3,5-tetrahydro-4H-pyrrolo[3,2-d]pyrimidin-4-one (185 mg) obtained by the method of Example 22, or a method pursuant to thereto, 4-bromo-N-(2-cyanoethyl)butanamide (131 mg) obtained by the method of Reference Example 49, or a method pursuant to thereto, sodium iodide (75 mg), triethylamine (140 μl) and N,N-dimethylformamide (5 ml) was heated to 100° C., and was stirred for 12 hours. The reaction mixture was returned to room temperature, and then was diluted with ethyl acetate (80 ml). The dilution was washed with water and saturated brine, dried over anhydrous magnesium sulfate, and then concentrated under reduced pressure. The resulting residue was purified by chromatography, and thus the title compound (228 mg) was obtained as a yellowish white solid.

¹H NMR (400 MHz, DMSO-d₆) δ ppm 1.26 (3H, t, J=7.6 Hz), 1.86 (2H, tt, J=7.3, 7.1 Hz), 2.18 (2H, t, J=7.3 Hz), 2.60 (2H, t, J=6.6 Hz), 2.63 (2H, q, J=7.6 Hz), 3.07 (2H, t, J=7.1 Hz), 3.24 (2H, td, J=6.6, 5.8 Hz), 4.87 (2H, q, J=8.8 Hz), 7.15-7.22 (3H, m), 7.32 (2H, d, J=8.8 Hz), 8.22 (1H, t, J=5.8 Hz), 11.80(1H, s).

### Example 172

### N-(2-cyanoethyl)-4-({7-methyl-4-oxo-3-[4-(2,2,2-trifluoroethoxy)phenyl]-4,5-dihydro-3H-pyrrolo[3,2-d]pyrimidin-2-yl}sulfanyl)butanamide

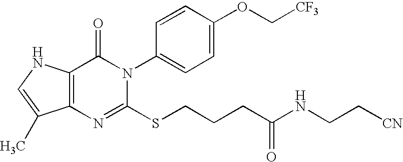

A mixture of 7-methyl-2-thioxo-3-[4-(2,2,2-trifluoroethoxy)phenyl]-1,2,3,5-tetrahydro-4H-pyrrolo[3,2-d]pyrimidin-4-one (170 mg) obtained by the method of Example 23, or a method pursuant to thereto, 4-bromo-N-(2-cyanoethyl)butanamide (126 mg) obtained by the method of Reference Example 49, or a method pursuant to thereto, sodium iodide (72 mg), triethylamine (133 μl) and N,N-dimethylformamide (5 ml) was heated to 100° C., and was stirred for 18 hours. The reaction mixture was returned to room temperature, and then was diluted with ethyl acetate (80 ml). The dilution was washed with water and saturated brine, dried over anhydrous magnesium sulfate, and then concentrated under reduced pressure. The resulting residue was purified by chromatography, and thus the title compound (49 mg) was obtained as a white solid.

¹H NMR (300 MHz, DMSO-d₆) δ ppm 1.86 (2H, tt, J=7.3, 7.1 Hz), 2.18 (2H, t, J=7.3 Hz), 2.17 (3H, s), 2.60 (2H, t, J=6.5 Hz), 3.09 (2H, t, J=7.1 Hz), 3.24 (2H, td, J=6.5, 5.7 Hz), 4.87 (2H, q, J=8.9 Hz), 7.18 (2H, d, J=9.1 Hz), 7.19 (1H, s), 7.31 (2H, d, J=9.1 Hz), 8.20 (1H, t, J=5.7 Hz), 11.79 (1H, s).

### Example 173

### N-(2-cyanoethyl)-2-{[3-(4-ethoxyphenyl)-4-oxo-4,7-dihydro-3H-pyrrolo[2,3-d]pyrimidin-2-yl]sulfanyl}acetamide

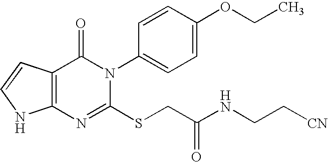

A mixture of 3-(4-ethoxyphenyl)-2-thioxo-1,2,3,7-tetrahydro-4H-pyrrolo[2,3-d]pyrimidin-4-one (144 mg) obtained by the method of Example 24, or a method pursuant to thereto, 2-chloro-N-(2-cyanoethyl)acetamide (80 mg) obtained by the method of Reference Example 40, or a method pursuant to thereto, triethylamine (140 μl) and acetonitrile (3 ml) was heated to reflux for 2 hours. The reaction mixture was returned to room temperature, and then was diluted with ethyl acetate (80 ml). The dilution was washed with water and saturated brine, dried over anhydrous magnesium sulfate, and then concentrated under reduced pressure. The resulting residue was purified by chromatography, and thus the title compound (171 mg) was obtained as a white solid.

¹H NMR (400 MHz, DMSO-d₆) δ ppm 1.37 (3H, t, J=7.0 Hz), 2.63 (2H, t, J=6.6 Hz), 3.29 (2H, td, J=6.6, 5.8 Hz), 3.80 (2H, s), 4.10 (2H, q, J=7.0 Hz), 6.43 (1H, d, J=3.4 Hz), 6.99 (1H, d, J=3.4 Hz), 7.06 (2H, d, J=8.8 Hz), 7.24 (2H, d, J=8.8 Hz), 8.43 (1H, t, J=5.8 Hz), 11.77 (1H, s).

### Example 174

### N-(2-cyanoethyl)-4-({3-[4-(2,2-dimethylpropoxy)phenyl]-4-oxo-4,7-dihydro-3H-pyrrolo[2,3-d]pyrimidin-2-yl}sulfanyl)butanamide

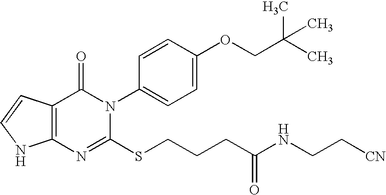

A mixture of 3-[4-(2,2-dimethylpropoxy)phenyl]-2-thioxo-1,2,3,7-tetrahydro-4H-pyrrolo[2,3-d]pyrimidin-4-one (235 mg) obtained by the method of Example 25, or a method pursuant to thereto, 4-bromo-N-(2-cyanoethyl)butanamide (175 mg) obtained by the method of Reference Example 49, or a method pursuant to thereto, potassium carbonate (193 mg) and N,N-dimethylformamide (5 ml) was stirred for 24 hours at room temperature. Subsequently, water (50 ml) was added to the reaction mixture, and the resulting mixture was extracted with ethyl acetate. The organic layer was washed with saturated brine, dried over anhydrous sodium sulfate, and then concentrated under reduced pressure. The resulting residue was purified by chromatography, and thus the title compound (111 mg) was obtained as a light brown solid.

¹H NMR (300 MHz, DMSO-d₆) δ ppm 1.03 (9 H, s), 1.84 (2H, tt, J=7.4, 7.0 Hz), 2.18 (2H, t, J=7.4 Hz), 2.60 (2H, t, J=6.4 Hz), 3.06 (2H, t, J=7.0 Hz), 3.24(2H, td, J=6.4, 5.5 Hz), 3.70 (2H, s), 6.42 (1H, dd, J=3.3, 2.0 Hz), 6.97 (1H, dd, J=3.3, 2.3 Hz), 7.05 (2H, d, J=9.0 Hz), 7.20 (2H, d, J=9.0 Hz), 8.21 (1H, t, J=5.6 Hz), 11.84(1H, br. s.).

### Example 175

### N-(2-cyanoethyl)-4-({4-oxo-3-[4-(2,2,2-trifluoroethoxy)phenyl]-4,5-dihydro-3H-pyrrolo[3,2-d]pyrimidin-2-yl}sulfanyl)butanamide

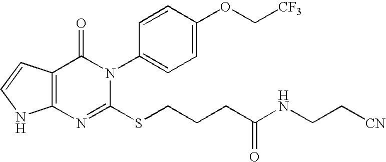

A mixture of 2-thioxo-3-[4-(2,2,2-trifluoroethoxy)phenyl]-1,2,3,7-tetrahydro-4H-pyrrolo[2,3-d]pyrimidin-4-one (171 mg) obtained by the method of Example 26, or a method pursuant to thereto, 4-bromo-N-(2-cyanoethyl)butanamide (131 mg) obtained by the method of Reference Example 49, or a method pursuant to thereto, triethylamine (209 μl), sodium iodide (75 mg) and N,N-dimethylformamide (5 ml) was heated to 100° C., and was stirred for 12 hours. The reaction mixture was returned to room temperature, and then was diluted with ethyl acetate (80 ml). The dilution was washed with water and saturated brine, dried over anhydrous magnesium sulfate, and then concentrated under reduced pressure. The resulting residue was purified by chromatography, and thus the title compound (93 mg) was obtained as a white solid.

¹H NMR (300 MHz, DMSO-d₆) δ ppm 1.85 (2H, tt, J=7.4, 7.1 Hz), 2.18 (2H, t, J=7.4 Hz), 2.60 (2H, t, J=6.4 Hz), 3.07 (2H, t, J=7.1 Hz), 3.24 (2H, td, J=6.4, 5.7 Hz), 4.86 (2H, q, J=8.8 Hz), 6.43 (1H, d, J=3.4 Hz), 6.97 (1H, d, J=3.4 Hz), 7.18 (2H, d, J=9.1 Hz), 7.30 (2H, d, J=9.1 Hz), 8.19 (1H, t, J=5.7 Hz), 11.85 (1H, s).

### Example 176

### N-(2-cyanoethyl)-4-({3-[4-(3,3-dimethylbutoxy)phenyl]-4-oxo-4,7-dihydro-3H-pyrrolo[2,3-d]pyrimidin-2-yl}sulfanyl)butanamide

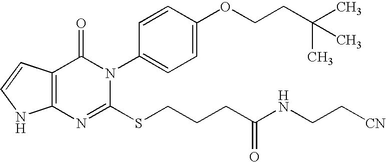

A mixture of 3-[4-(3,3-dimethylbutoxy)phenyl]-2-thioxo-1,2,3,7-tetrahydro-4H-pyrrolo[2,3-d]pyrimidin-4-one (536 mg) obtained by the method of Example 27, or a method pursuant to thereto, 4-bromo-N-(2-cyanoethyl)butanamide (416 mg) obtained by the method of Reference Example 49, or a method pursuant to thereto, potassium carbonate (441 mg) and N,N-dimethylformamide (5 ml) was stirred for 24 hours at room temperature. Subsequently, water (50 ml) was added to the reaction mixture, and the resulting mixture was extracted with ethyl acetate. The organic layer was washed with saturated brine, dried over anhydrous sodium sulfate, and then concentrated under reduced pressure. The resulting residue was purified by chromatography, and thus the title compound (203 mg) was obtained as a brown solid.

¹H NMR (300 MHz, DMSO-d₆) δ ppm 0.99 (9 H, s), 1.70 (2H, t, J=7.2 Hz), 1.84 (2H, tt, J=7.4, 7.0 Hz), 2.18 (2H, t, J=7.4 Hz), 2.60 (2H, t, J=6.4 Hz), 3.06(2H, t, J=7.0 Hz), 3.24 (2H, td, J=6.4, 5.8 Hz), 4.09 (2H, t, J=7.2 Hz), 6.42 (1H, dd, J=3.3, 2.1 Hz), 6.97 (1H, dd, J=3.3, 2.3 Hz), 7.05 (2H, d, J=9.0 Hz), 7.20 (2H, d, J=9.0 Hz), 8.19 (1H, t, J=5.8 Hz), 11.83 (1H, br. s.).

### Example 177

### N-(2-cyanoethyl)-4-[(3-{4-[(2,2-difluorocyclopropyl)methoxy]phenyl}-4-oxo-4,7-dihydro-3H-pyrrolo[2,3-d]pyrimidin-2-yl)sulfanyl]butanamide

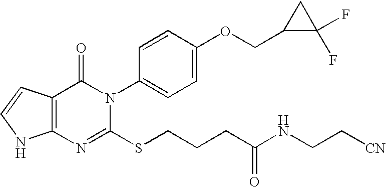

A mixture of 3-{4-[(2,2-difluorocyclopropyOmethoxy]phenyl}-2-thioxo-1,2,3,7-tetrahydro-4H-pyrrolo[2,3-d]pyrimidin-4-one (466 mg) obtained by the method of Example 28, or a method pursuant to thereto, 4-bromo-N-(2-cyanoethyl)butanamide (350 mg) obtained by the method of Reference Example 49, or a method pursuant to thereto, potassium carbonate (359 mg) and N,N-dimethylformamide (5 ml) was stirred for 24 hours at room temperature. Subsequently, water (50 ml) was added to the reaction mixture, and the resulting mixture was extracted with ethyl acetate. The organic layer was washed with saturated brine, dried over anhydrous sodium sulfate, and then concentrated under reduced pressure. The resulting residue was purified by chromatography, and thus the title compound (95 mg) was obtained as a brown solid.

¹H NMR (300 MHz, DMSO-d₆) δ ppm 1.44-1.63 (1H, m), 1.67-1.79 (1H, m), 1.85 (2H, tt, J=7.4, 7.0 Hz), 2.18 (2H, t, J=7.4 Hz), 2.21-2.39 (1H, m), 2.60(2H, t, J=6.4 Hz), 3.06 (2H, t, J=7.0 Hz), 3.24 (2H, td, J=6.4, 5.7 Hz), 3.98-4.31 (2H, m), 6.42 (1H, dd, J=3.4, 2.1 Hz), 6.97 (1H, dd, J=3.4, 2.3 Hz), 7.08 (2H, d, J=9.0 Hz), 7.23 (2H, d, J=9.0 Hz), 8.20 (1H, t, J=5.7 Hz), 11.84 (1H, br. s.).

### Example 178

### Tert-butyl 4-({4-oxo-3-[4-(2,2,2-trifluoroethoxy)phenyl]-4,7-dihydro-3H-pyrrolo[2,3-d]pyrimidin-2-yl}sulfanyl)butanoate

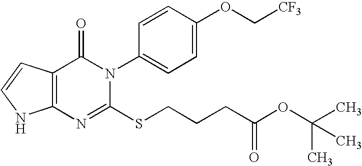

A mixture of 2-thioxo-3-[4-(2,2,2-trifluoroethoxy)phenyl]-1,2,3,7-tetrahydro-4H-pyrrolo[2,3-d]pyrimidin-4-one (341 mg) obtained by the method of Example 26, or a method pursuant to thereto, tert-butyl 4-bromobutanoate (267 mg), a 1 M aqueous solution of sodium hydrogen carbonate (1.1 ml) and N,N-dimethylformamide (10 ml) was heated to 100° C., and the resulting mixture was stirred for one hour. The reaction mixture was returned to room temperature, and then was diluted with ethyl acetate (200 ml). The dilution was washed with water and saturated brine, dried over anhydrous magnesium sulfate, and then concentrated under reduced pressure. The resulting residue was purified by chromatography, and then was washed with diethyl ether, and thus the title compound (445 mg) was obtained as a white solid.

¹H NMR (300 MHz, DMSO-d₆) δ ppm 1.37 (9 H, s), 1.83 (2H, tt, J=7.4, 7.0 Hz), 2.28 (2H, t, J=7.4 Hz), 3.06 (2H, t, J=7.0 Hz), 4.86 (2H, q, J=9.0 Hz), 6.43 (1H, d, J=3.4 Hz), 6.98 (1H, d, J=3.4 Hz), 7.18 (2H, d, J=9.1 Hz), 7.30 (2H, d, J=9.1 Hz), 11.85 (1H, s).

### Example 179

### Tert-butyl 4-({7-methyl-4-oxo-3-[4-(2,2,2-trifluoroethoxy)phenyl]-4,7-dihydro-3H-pyrrolo[2,3-d]pyrimidin-2-yl}sulfanyl)butanoate

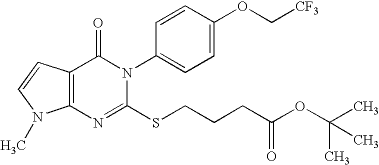

Tert-butyl 4-({4-oxo-3-[4-(2,2,2-trifluoroethoxy)phenyl]-4,7-dihydro-3H-pyrrolo[2,3-d]pyrimidin-2-yl}sulfanyl)butanoate (115 mg) obtained by the method of Example 178, or a method pursuant to thereto, was dissolved in N,N-dimethylformamide (5 ml), and potassium tert-butoxide (31.4 mg) and iodomethane (149 μl) were added sequentially to the solution. The mixture was stirred for 18 hours at room temperature. Subsequently, the reaction mixture was diluted with ethyl acetate (80 ml), and the dilution was washed with water and saturated brine, dried over anhydrous magnesium sulfate, and then concentrated under reduced pressure. The resulting residue was purified by chromatography, and thus the title compound (69 mg) was obtained as a white solid.

¹H NMR (300 MHz, DMSO-d₆) δ ppm 1.37 (9 H, s), 1.88 (2H, tt, J=7.3, 7.2 Hz), 2.30 (2H, t, J=7.3 Hz), 3.11 (2H, t, J=7.2 Hz), 3.73 (3H, s), 4.86 (2H, q, J=8.9 Hz), 6.44 (1H, d, J=3.4 Hz), 7.05 (1H, d, J=3.4 Hz), 7.18 (2H, d, J=9.0 Hz), 7.30 (2H, d, J=9.0 Hz).

### Example 180

### Tert-butyl 4-({7-ethyl-4-oxo-3-[4-(2,2,2-trifluoroethoxy)phenyl]-4,7-dihydro-3H-pyrrolo[2,3-d]pyrimidin-2-yl}sulfanyl)butanoate

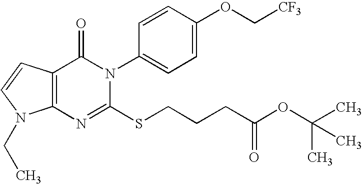

Tert-butyl 4-({4-oxo-3-[4-(2,2,2-trifluoroethoxy)phenyl]-4,7-dihydro-3H-pyrrolo[2,3-d]pyrimidin-2-yl}sulfanyl)butanoate (100 mg) obtained by the method of Example 178, or a method pursuant to thereto was dissolved in N,N-dimethylformamide (2 ml), and sodium hydride (60% in oil, 12 mg) and iodoethane (331 μl) were added sequentially to the solution. The mixture was stirred for one hour at room temperature under a nitrogen atmosphere. Subsequently, the reaction mixture was poured into a saturated aqueous solution of ammonium chloride (1 ml), and the resultant was diluted with ethyl acetate (80 ml). The dilution was washed with water and saturated brine, dried over anhydrous magnesium sulfate, and then concentrated under reduced pressure. The resulting residue was purified by chromatography, and thus the title compound (103 mg) was obtained as white needle-shaped crystals.

¹H NMR (300 MHz, CHLOROFORM-d) δ ppm 1.43 (9 H, s), 1.47 (3H, t, J=7.2 Hz), 2.01 (2H, tt, J=7.4, 7.2 Hz), 2.33 (2H, t, J=7.4 Hz), 3.12 (2H, t, J=7.2 Hz), 4.18 (2H, q, J=7.2 Hz), 4.41 (2H, q, J=8.0 Hz), 6.63 (1H, d, J=3.4 Hz), 6.77 (1H, d, J=3.4 Hz), 7.06 (2H, d, J=9.1 Hz), 7.24 (2H, d, J=9.1 Hz).

### Example 181

### 4-({7-Methyl-4-oxo-3-[4-(2,2,2-trifluoroethoxy)phenyl]-4,7-dihydro-3H-pyrrolo[2,3-d]pyrimidin-2-yl}sulfanyl)butanoic acid

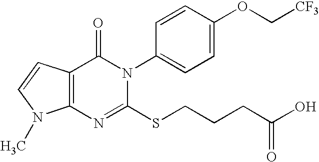

A mixture of tert-butyl 4-({7-methyl-4-oxo-3-[4-(2,2,2-trifluoroethoxy)phenyl]-4,7-dihydro-3H-pyrrolo[2,3-d]pyrimidin-2-yl}sulfanyl)butanoate (60 mg) obtained in Example 179, acetonitrile (2 ml) and 6 M hydrochloric acid (1 ml) was heated to reflux for one hour. The reaction mixture was returned to room temperature, and then was concentrated under reduced pressure. To the resulting residue, diethyl ether was added, and the mixture was filtered, and then the filtrate was concentrated under reduced pressure. Thus, the title compound (69 mg) was obtained.

¹H NMR (300 MHz, CHLOROFORM-d) δ ppm 2.05 (2H, tt, J=7.3, 7.1 Hz), 2.43 (2H, t, J=7.3 Hz), 3.17 (2H, t, J=7.1 Hz), 3.75 (3H, s), 4.41 (2H, q, J=8.1 Hz), 6.60 (1H, d, J=3.4 Hz), 6.74 (1H, d, J=3.4 Hz), 7.06 (2H, d, J=8.9 Hz), 7.21 (2H, d, J=8.9 Hz), 9.04 (1H, br. s.).

### Example 182

### 4-({7-Ethyl-4-oxo-3-[4-(2,2,2-trifluoroethoxy)phenyl]-4,7-dihydro-3H-pyrrolo[2,3-d]pyrimidin-2-yl}sulfanyl)butanoic acid

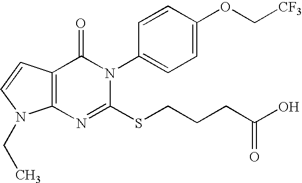

A mixture of tert-butyl 4-({7-ethyl-4-oxo-3-[4-(2,2,2-trifluoroethoxy)phenyl]-4,7-dihydro-3H-pyrrolo[2,3-d]pyrimidin-2-yl}sulfanyl)butanoate (100 mg) obtained in Example 180, acetonitrile (4 ml) and 6 M hydrochloric acid (2 ml) was stirred for 8 hours at room temperature. Saturated brine (10 ml) was added to the reaction mixture liquid, and the resulting mixture was extracted with ethyl acetate. The organic layer was washed with saturated brine, dried over anhydrous magnesium sulfate, and then concentrated under reduced pressure. A white solid obtained therefrom was recrystallized from a mixed solvent of ethyl acetate/hexane. Thus, the title compound (81 mg) was obtained as a white solid.

¹H NMR (300 MHz, DMSO-d₆) δ ppm 1.38 (3H, t, J=7.2 Hz), 1.89 (2H, tt, J=7.3, 7.2 Hz), 2.31 (2H, t, J=7.3 Hz), 3.11 (2H, t, J=7.2 Hz), 4.17 (2H, q, J=7.2 Hz), 4.86 (2H, q, J=8.9 Hz), 6.44 (1H, d, J=3.4 Hz), 7.11 (1H, d, J=3.4 Hz), 7.18 (2H, d, J=9.0 Hz), 7.31 (2H, d, J=9.0 Hz), 12.13 (1H, br. s.).

### Example 183

### N-(2-cyanoethyl)-4-({3-[4-(cyclobutylmethoxy)phenyl]-4-oxo-4,7-dihydro-3H-pyrrolo[2,3-d]pyrimidin-2-yl}sulfanyl)butanamide

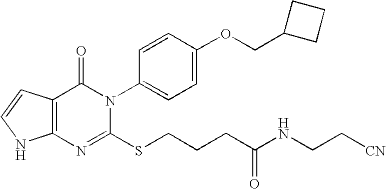

A mixture of 3-[4-(cyclobutylmethoxy)phenyl]-2-thioxo-1,2,3,7-tetrahydro-4H-pyrrolo[2,3-d]pyrimidin-4-one (346 mg) obtained by the method of Example 29, or a method pursuant to thereto, 4-bromo-N-(2-cyanoethyl)butanamide (285 mg) obtained by the method of Reference Example 49, or a method pursuant to thereto, potassium carbonate (304 mg) and N,N-dimethylformamide (5 ml) was stirred for 24 hours at room temperature. Subsequently, water (50 ml) was added to the reaction mixture, and the resulting mixture was extracted with ethyl acetate. The organic layer was washed with saturated brine, dried over anhydrous sodium sulfate, and then concentrated under reduced pressure. The resulting residue was purified by chromatography, and thus the title compound (73 mg) was obtained as a yellowish white solid.

¹H NMR (300 MHz, DMSO-d₆) δ ppm 1.73-2.02 (6 H, m), 2.04-2.14 (2H, m), 2.18 (2H, t, J=7.4 Hz), 2.60 (2H, t, J=6.4 Hz), 2.67-2.86 (1H, m), 3.06 (2H, t, J=7.0 Hz), 3.24 (2H, td, J=6.4, 5.7 Hz), 4.02 (2H, d, J=6.8 Hz), 6.42 (1H, dd, J=3.3; 1.9 Hz), 6.97 (1H, dd, J=3.3, 2.3 Hz), 7.04 (2H, d, J=9.0 Hz), 7.20 (2H, d, J=9.0 Hz), 8.20 (1H, t, J=5.7 Hz), 11.84 (1H, br. s.).

### Example 184

### 4-{[3-(4-Butoxyphenyl)-4-oxo-4,7-dihydro-3H-pyrrolo[2,3-d]pyrimidin-2-yl]sulfanyl}-N-(2-cyanoethyl)butanamide

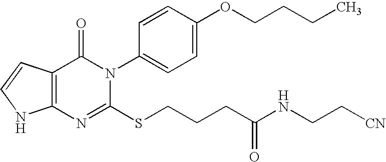

A mixture of 3-(4-butoxyphenyl)-2-thioxo-1,2,3,7-tetrahydro-4H-pyrrolo[2,3-d]pyrimidin-4-one (254 mg) obtained by the method of Example 30, or a method pursuant to thereto, 4-bromo-N-(2-cyanoethyl)butanamide (219 mg) obtained by the method of Reference Example 49, or a method pursuant to thereto, potassium carbonate (220 mg) and N,N-dimethylformamide (5 ml) was stirred for 24 hours at room temperature. Subsequently, water (50 ml) was added to the reaction mixture, and the resulting mixture was extracted with ethyl acetate. The organic layer was washed with saturated brine, dried over anhydrous sodium sulfate, and then concentrated under reduced pressure. The resulting residue was purified by chromatography, and was recrystallized from a mixed solvent of ethyl acetate/hexane. Thus, the title compound (71 mg) was obtained as a brown solid.

¹H NMR (300 MHz, DMSO-d₆) δ ppm 0.96 (3H, t, J=7.4 Hz), 1.39-1.55 (2H, m), 1.67-1.79 (2H, m), 1.84 (2H, tt, J=7.4, 7.0 Hz), 2.18 (2H, t, J=7.4 Hz), 2.60 (2H, t, J=6.4 Hz), 3.06 (2H, t, J=7.0 Hz), 3.24 (2H, td, J=6.4, 5.9 Hz), 4.04 (2H, t, J=6.4 Hz), 6.42 (1H, dd, J=3.3, 1.8 Hz), 6.97 (1H, dd, J=3.3, 2.3 Hz), 7.04 (2H, d, J=9.0 Hz), 7.20 (2H, d, J=9.0 Hz), 8.19 (1H, t, J=5.9 Hz), 11.83 (1H, br. s.).

### Example 185

### N-(2-cyanoethyl)-4-({3-[4-(cyclopropylmethoxy)phenyl]-4-oxo-4,7-dihydro-3H-pyrrolo[2,3-d]pyrimidin-2-yl}sulfanyl)butanamide

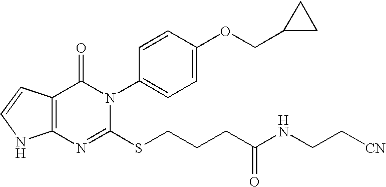

A mixture of 3-[4-(cyclopropylmethoxy)phenyl]-2-thioxo-1,2,3,7-tetrahydro-4H-pyrrolo[2,3-d]pyrimidin-4-one (403 mg) obtained by the method of Example 31, or a method pursuant to thereto, 4-bromo-N-(2-cyanoethyl)butanamide (350 mg) obtained by the method of Reference Example 49, or a method pursuant to thereto, potassium carbonate (359 mg) and N,N-dimethylformamide (5 ml) was stirred for 24 hours at room temperature. Subsequently, water (50 ml) was added to the reaction mixture, and the resulting mixture was extracted with ethyl acetate. The organic layer was washed with saturated brine, dried over anhydrous sodium sulfate, and then concentrated under reduced pressure. The resulting residue was purified by chromatography, and was recrystallized from a mixed solvent of ethyl acetate/hexane. Thus, the title compound (88 mg) was obtained as a brown solid.

¹H NMR (300 MHz, DMSO-d₆) δ ppm 0.32-0.41 (2H, m), 0.55-0.65 (2H, m), 1.14-1.35 (1H, m), 1.84 (2H, tt, J=7.4, 7.0 Hz), 2.18 (2H, t, J=7.4 Hz), 2.60 (2H, t, J=6.4 Hz), 3.05 (2H, t, J=7.0 Hz), 3.24 (2H, td, J=6.4, 5.7 Hz), 3.88 (2H, d, J=6.8 Hz), 6.42 (1H, d, J=3.4 Hz), 6.97 (1H, d, J=3.4 Hz), 7.03 (2H, d, J=9.0 Hz), 7.20 (2H, d, J=9.0 Hz), 8.19 (1H, t, J=5.7 Hz), 11.83 (1H, s).

### Example 186

### N-(2-cyanoethyl)-4-({4-oxo-3-[4-(4,4,4-trifluorobutoxy)phenyl]-4,7-dihydro-3H-pyrrolo[2,3-d]pyrimidin-2-yl}sulfanyl)butanamide

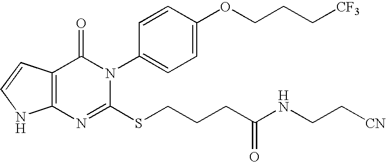

A mixture of 2-thioxo-3-[4-(4,4,4-trifluorobutoxy)phenyl]-1,2,3,7-tetrahydro-4H-pyrrolo[2,3-d]pyrimidin-4-one (213 mg) obtained by the method of Example 32, or a method pursuant to thereto, 4-bromo-N-(2-cyanoethyl)butanamide (153 mg) obtained by the method of Reference Example 49, or a method pursuant to thereto, potassium carbonate (166 mg) and N,N-dimethylformamide (5 ml) was stirred for 24 hours at room temperature. Subsequently, water (50 ml) was added to the reaction mixture, and the resulting mixture was extracted with ethyl acetate. The organic layer was washed with saturated brine, dried over anhydrous sodium sulfate, and then concentrated under reduced pressure. The resulting residue was purified by chromatography, and was recrystallized from a mixed solvent of ethyl acetate/hexane. Thus, the title compound (63 mg) was obtained as a brown solid.

¹H NMR (300 MHz, DMSO-d₆) δ ppm 1.84 (2H, tt, J=7.4, 7.0 Hz), 1.93-2.08 (2H, m), 2.18 (2H, t, J=7.4 Hz), 2.36-2.53 (2H, m), 2.60 (2H, t, J=6.4 Hz), 3.06 (2H, t, J=7.0 Hz), 3.24 (2H, td, J=6.4, 5.6 Hz), 4.11 (2H, t, J=6.1 Hz), 6.42 (1H, dd, J=3.4, 1.9 Hz), 6.97 (1H, dd, J=3.4, 2.4 Hz), 7.06 (2H, d, J=9.0 Hz), 7.23 (2H, d, J=9.0 Hz), 8.20 (1H, t, J=5.6 Hz), 11.84 (1H, br. s.).

### Example 187

### N-(2-cyanoethyl)-4-[(3-{4-[(2-methylcyclopropyl)methoxy]phenyl}-4-oxo-4,7-dihydro-3H-pyrrolo[2,3-d]pyrimidin-2-yl)sulfanyl]butanamide

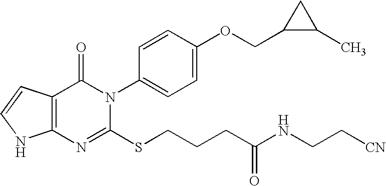

A mixture of 3-{4-[(2-methylcyclopropyl)methoxy]phenyl}-2-thioxo-1,2,3,7-tetrahydro-4H-pyrrolo[2,3-d]pyrimidin-4-one (268 mg) obtained by the method of Example 33, or a method pursuant to thereto, 4-bromo-N-(2-cyanoethyl)butanamide (219 mg) obtained by the method of Reference Example 49, or a method pursuant to thereto, potassium carbonate (331 mg) and N,N-dimethylformamide (5 ml) was stirred for 24 hours at room temperature. Subsequently, water (50 ml) was added to the reaction mixture, and the resulting mixture was extracted with ethyl acetate. The organic layer was washed with saturated brine, dried over anhydrous sodium sulfate, and then concentrated under reduced pressure. The resulting residue was purified by chromatography, and was recrystallized from a mixed solvent of ethyl acetate/hexane. Thus, the title compound (41 mg) was obtained as a yellowish white solid.

¹H NMR (300 MHz, DMSO-d₆) δ ppm 0.28-0.42 (1H, m), 0.44-0.60 (1H, m), 0.69-0.89 (1H, m), 0.91-1.04 (1H, m), 1.07 (3H, d, J=6.0 Hz), 1.84 (2H, tt, J=7.4, 7.0 Hz), 2.18 (2H, t, J=7.4 Hz), 2.60 (2H, t, J=6.4 Hz), 3.05 (2H, t, J=7.0 Hz), 3.24 (2H, td,

J=6.4, 5.7 Hz), 3.77-4.00 (2H, m), 6.42 (1H, dd, J=3.4, 1.9 Hz), 6.97 (1H, dd, J=3.4, 2.3 Hz), 7.02 (2H, d, J=8.9 Hz), 7.19 (2H, d, J=8.9 Hz), 8.19 (1H, t, J=5.7 Hz), 11.83 (1H, br. s.).

### Example 188

### N-(2-cyanoethyl)-4-[(3-{4-[(1-methylcyclopropyl)methoxy]phenyl}-4-oxo-4,7-dihydro-3H-pyrrolo[2,3-d]pyrimidin-2-yl)sulfanyl]butanamide

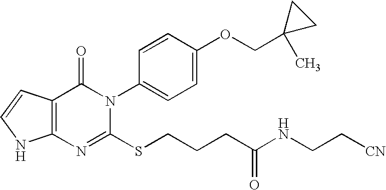

A mixture of 3-{4-[(1-methylcyclopropyl)methoxy]phenyl}-2-thioxo-1,2,3,7-tetrahydro-4H-pyrrolo[2,3-d]pyrimidin-4-one (309 mg) obtained by the method of Example 34, or a method pursuant to thereto, 4-bromo-N-(2-cyanoethyl)butanamide (241 mg) obtained by the method of Reference Example 49, or a method pursuant to thereto, potassium carbonate (247 mg) and N,N-dimethylformamide (5 ml) was stirred for 24 hours at room temperature. Subsequently, water (50 ml) was added to the reaction mixture, and the resulting mixture was extracted with ethyl acetate. The organic layer was washed with saturated brine, dried over anhydrous sodium sulfate, and then concentrated under reduced pressure. The resulting residue was purified by chromatography, and was recrystallized from a mixed solvent of ethyl acetate/hexane. Thus, the title compound (38 mg) was obtained as a yellowish white solid.

¹H NMR (300 MHz, DMSO-d₆) δ ppm 0.36-0.48 (2H, m), 0.49-0.62 (2H, m), 1.21 (3H, s), 1.84 (2H, tt, J=7.4, 7.0 Hz), 2.18 (2H, t, J=7.4 Hz), 2.60 (2H, t, J=6.4 Hz), 3.05 (2H, t, J=7.0 Hz), 3.24 (2H, td, J=6.4, 5.7 Hz), 3.82 (2H, s), 6.42 (1H, dd, J=3.3, 1.5 Hz), 6.97 (1H, dd, J=3.3, 2.1 Hz), 7.03 (2H, d, J=8.9 Hz), 7.19 (2H, d, J=8.9 Hz), 8.20 (1H, t, J=5.7 Hz), 11.83 (1H, br. s.).

### Example 189

### N-(2-cyanoethyl)-4-({4-oxo-3-[4-(3,3,3-trifluoropropoxy)phenyl]-4,7-dihydro-3H-pyrrolo[2,3-d]pyrimidin-2-yl}sulfanyl)butanamide

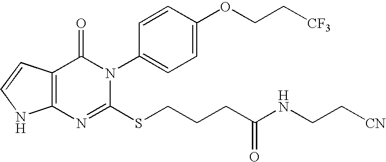

A mixture of 2-thioxo-3-[4-(3,3,3-trifluoropropoxy)phenyl]-1,2,3,7-tetrahydro-4H-pyrrolo[2,3-d]pyrimidin-4-one (324 mg) obtained by the method of Example 35, or a method pursuant to thereto, 4-bromo-N-(2-cyanoethyl)butanamide (219 mg) obtained by the method of Reference Example 49, or a method pursuant to thereto, potassium carbonate (248 mg) and N,N-dimethylformamide (5 ml) was stirred for 24 hours at room temperature.

Subsequently, water (50 ml) was added to the reaction mixture, and the resulting mixture was extracted with ethyl acetate. The organic layer was washed with saturated brine, dried over anhydrous sodium sulfate, and then concentrated under reduced pressure. The resulting residue was purified by chromatography, and was recrystallized from a mixed solvent of ethyl acetate/hexane. Thus, the title compound (103 mg) was obtained as a brown solid.

¹H NMR (300 MHz, DMSO-d₆) δ ppm 1.85 (2H, tt, J=7.4, 7.0 Hz), 2.18 (2H, t, J=7.4 Hz), 2.60 (2H, t, J=6.4 Hz), 2.74-2.93 (2H, m), 3.06 (2H, t, J=7.0 Hz), 3.24 (2H, td, J=6.4, 5.7 Hz), 4.28 (2H, t, J=5.9 Hz), 6.42 (1H, dd, J=3.4, 1.9 Hz), 6.97 (1H, dd, J=3.4, 2.3 Hz), 7.09 (2H, d, J=9.1 Hz), 7.24 (2H, d, J=9.1 Hz), 8.20 (1H, t, J=5.7 Hz), 11.84 (1H, br. s.).

### Example 190

### N-(2-cyanoethyl)-4-({3-[4-(3-methylbutoxy)phenyl]-4-oxo-4,7-dihydro-3H-pyrrolo[2,3-d]pyrimidin-2-yl}sulfanyl)butanamide

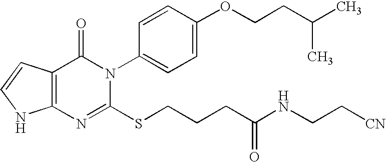

A mixture of 3-[4-(3-methylbutoxy)phenyl]-2-thioxo-1,2,3,7-tetrahydro-4H-pyrrolo[2,3-d]pyrimidin-4-one (204 mg) obtained by the method of Example 36, or a method pursuant to thereto, 4-bromo-N-(2-cyanoethyl)butanamide (153 mg) obtained by the method of Reference Example 49, or a method pursuant to thereto, potassium carbonate (116 mg) and N,N-dimethylformamide (5 ml) was stirred for 24 hours at room temperature. Subsequently, water (50 ml) was added to the reaction mixture, and the resulting mixture was extracted with ethyl acetate. The organic layer was washed with saturated brine, dried over anhydrous sodium sulfate, and then concentrated under reduced pressure. The resulting residue was purified by chromatography, and was recrystallized from a mixed solvent of ethyl acetate/hexane. Thus, the title compound (37 mg) was obtained as a brown solid.

¹H NMR (300 MHz, DMSO-d₆) δ ppm 0.96 (6 H, d, J=6.5 Hz), 1.65 (2H, dt, J=6.8, 6.6 Hz), 1.74-1.96 (3H, m), 2.18 (2H, t, J=7.4 Hz), 2.60 (2H, t, J=6.4 Hz), 3.05 (2H, t, J=7.0 Hz), 3.24 (2H, td, J=6.4, 5.7 Hz), 4.06 (2H, t, J=6.6 Hz), 6.42 (1H, d, J=3.3 Hz), 6.97 (1H, dd, J=3.3, 1.5 Hz), 7.05 (2H, d, J=9.0 Hz), 7.20 (2H, d, J=9.0 Hz), 8.21 (1H, t, J=5.7 Hz), 11.84 (1H, br. s.).

### Example 191

### 2-{[3-(2-Methoxyethoxy)propyl]sulfanyl}-3-[4-(2,2,2-trifluoroethoxy)phenyl]-3,7-dihydro-4H-pyrrolo[2,3-d]pyrimidin-4-one

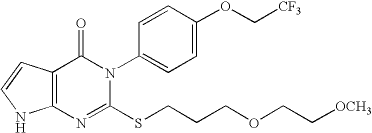

A 1 M aqueous solution of sodium hydrogen carbonate (0.44 ml) was added to a mixture of 2-thioxo-3-[4-(2,2,2-trifluoroethoxy)phenyl]-1,2,3,7-tetrahydro-4H-pyrrolo[2,3-d]pyrimidin-4-one (150 mg) obtained by the method of Example 26, or a method pursuant to thereto, 1-bromo-3-(2-methoxyethoxy)propane (130 mg), sodium iodide (66 mg) and N,N-dimethylformamide (5 ml), and the resulting mixture was stirred for 2 hours at 100° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (163 mg) was obtained as a white powder.

¹H NMR (300 MHz, DMSO-d₆) δ ppm 1.83 (2H, m), 3.07 (2H, t, J=7.2 Hz), 3.21 (3H, s), 3.36-3.51 (6 H, m), 4.86 (2H, q, J=8.8 Hz), 6.43 (1H, d, J=3.4 Hz), 6.98 (1H, d, J=3.4 Hz), 7.18 (2H, d, J=8.7 Hz), 7.30 (2H, d, J=8.7 Hz), 11.87 (1H, br. s.).

### Example 192

### 2-{[2-(2-Ethoxyethoxy)ethyl]sulfanyl}-3-[4-(2,2,2-trifluoroethoxy)phenyl]-3,7-dihydro-4H-pyrrolo[2,3-d]pyrimidin-4-one

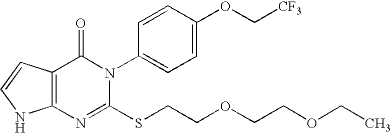

A 1 M aqueous solution of sodium hydrogen carbonate (0.44 ml) was added to a mixture of 2-thioxo-3-[4-(2,2,2-trifluoroethoxy)phenyl]-1,2,3,7-tetrahydro-4H-pyrrolo[2,3-d]pyrimidin-4-one (150 mg) obtained by the method of Example 26, or a method pursuant to thereto, 1-bromo-2-(2-ethoxyethoxy)ethane (130 mg), sodium iodide (66 mg) and N,N-dimethylformamide (5 ml), and the resulting mixture was stirred for 2 hours at 100° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (128 mg) was obtained as a white powder.

¹H NMR (300 MHz, DMSO-d₆) δ ppm 1.06 (3H, t), 3.25 (2H, t, J=6.4 Hz), 3.34-3.47 (4H, m), 3.47-3.54 (2H, m), 3.61 (2H, t, J=6.4 Hz), 4.86 (2H, q, J=8.7 Hz), 6.43 (1H, d, J=3.4 Hz), 6.98 (1H, d, J=3.4 Hz), 7.18 (2H, d, J=8.7 Hz), 7.30 (2H, d, J=8.7 Hz), 11.89 (1H, br. s.).

### Example 193

### N-(2-cyanoethyl)-4-({7-ethyl-4-oxo-3-[4-2,2,2-trifluoroethoxy)phenyl]-4,7-dihydro-3H-pyrrolo[2,3-d]pyrimidin-2-yl}sulfanyl)butanamide

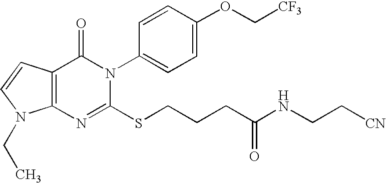

4-({7-Ethyl-4-oxo-3-[4-(2,2,2-trifluoroethoxy)phenyl]-4,7-dihydro-3H-pyrrolo[2,3-d]pyrimidin-2-yl}sulfanyl)butanoic acid (80 mg) obtained by the method of Example 182, or a method pursuant to thereto was dissolved in N,N-dimethylformamide (2 ml), and 1-ethyl-3-(3-dimethylaminopropyl)carbodiimide hydrochloride (67.3 mg), 1-hydroxybenzotriazole (28.5 mg) and 3-aminopropanenitrile (250 μl) were added to the solution. The mixture was stirred for 24 hours at room temperature, and then was diluted with ethyl acetate (80 ml). The dilution was washed with 0.1 M hydrochloric acid and saturated brine, dried over anhydrous magnesium sulfate, and then concentrated under reduced pressure. The resulting residue was purified by chromatography, and thus the title compound (26.3 mg) was obtained as a white solid.

¹H NMR (400 MHz, DMSO-d₆) δ ppm 1.38 (3H, t, J=7.2 Hz), 1.89 (2H, J=7.4, 7.1 Hz), 2.19 (2H, t, J=7.4 Hz), 2.61 (2H, t, J=6.4 Hz), 3.08 (2H, t, J=7.1 Hz), 3.25 (2H, td, J=6.4, 5.7 Hz), 4.17 (2H, q, J=7.2 Hz), 4.87 (2H, q, J=8.8 Hz), 6.44 (1H, d, J=3.4 Hz), 7.12 (1H, d, J=3.4 Hz), 7.18 (2H, d, J=8.8 Hz), 7.31 (2H, d, J=8.8 Hz), 8.23 (1H, t, J=5.7 Hz).

### Example 194

### 2-(Ethylsulfanyl)-3-[4-(2,2,2-trifluoroethoxy)phenyl]-3,7-dihydro-4H-pyrrolo[2,3-d]pyrimidin-4-one

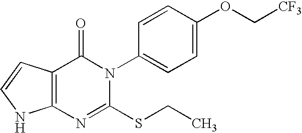

A mixture of 2-thioxo-3-[4-(2,2,2-trifluoroethoxy)phenyl]-1,2,3,7-tetrahydro-4H-pyrrolo[2,3-d]pyrimidin-4-one (900 mg) obtained by the method of Example 26, or a method pursuant to thereto, a 1 M aqueous solution of sodium hydrogen carbonate (2.64 ml), iodoethane (1.07 ml) and N,N-dimethylformamide (26 ml) was heated to 40° C., and the resulting mixture was stirred for 30 minutes. The reaction mixture was returned to room temperature, and then was diluted with ethyl acetate (150 ml). The dilution was washed with water and saturated brine, dried over anhydrous magnesium sulfate, and then concentrated under reduced pressure. The resulting residue was purified by chromatography, and then was washed with a mixed solvent of diethyl ether/hexane. Thus, the title compound (871 mg) was obtained as a white powder.

¹H NMR (300 MHz, DMSO-d₆) δ ppm 1.25 (3H, t, J=7.4 Hz), 3.04 (2H, q, J=7.4 Hz), 4.86 (2H, q, J=8.9 Hz), 6.43 (1H, d, J=3.4 Hz), 6.98 (1H, d, J=3.4 Hz), 7.18 (2H, d, J=9.1 Hz), 7.29 (2H, d, J=9.1 Hz), 11.87 (1H, s).

### Example 195

### N-(2-cyanoethyl)-4-(7-methyl-4-oxo-3-[4-(2,2,2-trifluoroethoxy)phenyl]-4,7-dihydro-3H-pyrrolo[2,3-d]pyrimidin-2-yl}sulfanyl)butanamide

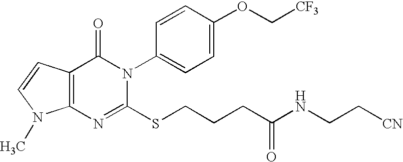

4-({7-Methyl-4-oxo-3-[4-(2,2,2-trifluoroethoxy)phenyl]-4,7-dihydro-3H-pyrrolo[2,3-d]pyrimidin-2-yl}sulfanyl)butanoic acid (60 mg) obtained by the method of Example 181, or a method pursuant to thereto was dissolved in N,N-dimethylformamide (2 ml). 1-Ethyl-3-(3-dimethylaminopropyl)carbodiimide hydrochloride (52.1 mg), 1-hydroxybenzotriazole (18.4 mg) and 3-aminopropanenitrile (524 μl) were added to the solution. The mixture was stirred overnight at room temperature, and then was diluted with ethyl acetate (80 ml). The dilution was washed sequentially with 1 M hydrochloric acid, water, a saturated aqueous solution of sodium hydrogen carbonate and saturated brine, dried over anhydrous magnesium sulfate, and then concentrated under reduced pressure. The resulting residue was purified by chromatography, and thus the title compound (9.9 mg) was obtained as a white solid.

¹H NMR (300 MHz, DMSO-d₆) δ ppm 1.89 (2H, tt, J=7.4, 7.1 Hz), 2.19 (2H, t, J=7.4 Hz), 2.61 (2H, t, J=6.4 Hz), 3.10 (2H, t, J=7.1 Hz), 3.25 (2H, td, J=6.4, 5.7 Hz), 3.74 (3H, s), 4.86 (2H, q, J=9.0 Hz), 6.44 (1H, d, J=3.4 Hz), 7.05 (1H, d, J=3.4 Hz), 7.18 (2H, d, J=9.1 Hz), 7.29 (2H, d, J=9.1 Hz), 8.22 (1H, t, J=5.7 Hz).

### Example 196

### Tert-butyl 4-({7-(2-{[tert-butyl(dimethyl)silyl]oxy}ethyl)-4-oxo-3-[4-(2,2,2-trifluoroethoxy)phenyl]-4,7-dihydro-3H-pyrrolo[2,3-d]pyrimidin-2-yl}sulfanyl)butanoate

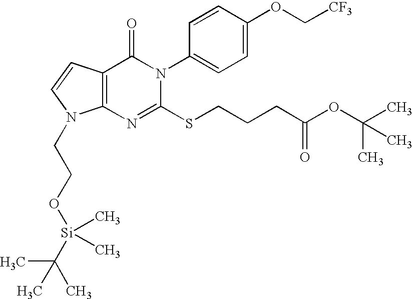

Tert-butyl 4-({4-oxo-3-[4-(2,2,2-trifluoroethoxy)phenyl]-4,7-dihydro-3H-pyrrolo[2,3-d]pyrimidin-2-yl}sulfanyl)butanoate (100 mg) obtained by the method of Example 178, or a method pursuant to thereto was dissolved in N,N-dimethylformamide (2 ml). Sodium hydride (60% in oil, 12 mg) and (2-bromoethoxy)(tert-butyl)dimethylsilane (58 μl) were added sequentially to the solution, and the resulting mixture was stirred for one hour at room temperature under a nitrogen atmosphere. The reaction mixture was poured into a saturated aqueous solution of ammonium chloride (3 ml), added with water (5 ml), and extracted with ethyl acetate (80 ml). The organic layer was washed with water and saturated brine, dried over anhydrous magnesium sulfate, and then concentrated under reduced pressure. The resulting residue was purified by chromatography, and thus the title compound (91 mg) was obtained as a colorless oily substance.

¹H NMR (300 MHz, CHLOROFORM-d) δ ppm -0.04 (6 H, s), 0.86 (9 H, s), 1.43 (9 H, s), 2.00 (2H, tt, J=7.3, 7.1 Hz), 2.31 (2H, t, J=7.3 Hz), 3.11 (2H, t, J=7.1 Hz), 3.92 (2H, t, J=5.5 Hz), 4.25 (2H, t, J=5.5 Hz), 4.41 (2H, q, J=8.1 Hz), 6.61 (1H, d, J=3.4 Hz), 6.84 (1H, d, J=3.4 Hz), 7.06 (2H, d, J=9.0 Hz), 7.23 (2H, d, J=9.0 Hz).

### Example 197

### 4-({7-(2-Hydroxyethyl)-4-oxo-3-[4-(2,2,2-trifluoroethoxy)phenyl]-4,7-dihydro-3H-pyrrolo[2,3-d]pyrimidin-2-yl}sulfanyl)butanoic acid

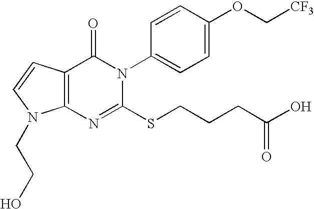

A mixture of tert-butyl 4-({7-(2-{[tert-butyl(dimethyl)silyl]oxy}ethyl)-4-oxo-3-[4-(2,2,2-trifluoroethoxy)phenyl]-4,7-dihydro-3H-pyrrolo[2,3-d]pyrimidin-2-yl}sulfanyl)butanoate (91 mg) obtained in Example 196, acetonitrile (4 ml) and 6 M hydrochloric acid (2 ml) was stirred for 8 hours at room temperature. Saturated brine (10 ml) was added to the reaction mixture liquid, and the mixture was extracted with ethyl acetate. The organic layer was washed with saturated brine, dried over anhydrous magnesium sulfate, and then concentrated under reduced pressure. A white solid obtained therefrom was recrystallized from a mixed solvent of ethyl acetate/hexane. Thus, the title compound (58.4 mg) was obtained as a white solid.

¹H NMR (300 MHz, DMSO-d₆) δ ppm 1.87 (2H, tt, J=7.2, 7.1 Hz), 2.30 (2H, t, J=7.2 Hz), 3.10 (2H, t, J=7.1 Hz), 3.75 (2H, t, J=5.5 Hz), 4.19 (2H, t, J=5.5 Hz), 4.86 (2H, q, J=8.9 Hz), 6.43 (1H, d, J=3.4 Hz), 7.08 (1H, d, J=3.4 Hz), 7.18 (2H, d, J=9.0 Hz), 7.30 (2H, d, J=9.0 Hz).

### Example 198

### N-(2-cyanoethyl)-4-({7-(2-hydroxyethyl)-4-oxo-3-[4-(2,2,2-trifluoroethoxy)phenyl]-4,7-dihydro-3H-pyrrolo[2,3-d]pyrimidin-2-yl}sulfanyl)butanamide

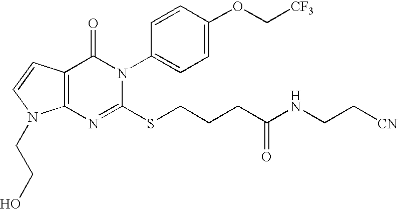

4-({7-(2-Hydroxyethyl)-4-oxo-3-[4-(2,2,2-trifluoroethoxy)phenyl]-4,7-dihydro-3H-pyrrolo[2,3-d]pyrimidin-2-yl}sulfanyl)butanoic acid (56 mg) obtained by the method of Example 197, or a method pursuant to thereto was dissolved in N,N-dimethylformamide (2 ml). 1-Ethyl-3-(3-dimethylaminopropyl)carbodiimide hydrochloride (45.5 mg), 1-hydroxybenzotriazole (19.3 mg) and 3-aminopropanenitrile (250 μl) were added to the solution. The resulting mixture was stirred overnight at room temperature, and then was diluted with ethyl acetate (80 ml). The dilution was washed sequentially with 0.1 M hydrochloric acid and saturated brine, dried over anhydrous magnesium sulfate, and then concentrated under reduced pressure. The resulting residue was purified by chromatography, and thus the title compound (27.3 mg) was obtained as a white solid.

¹H NMR (300 MHz, DMSO-d₆) δ ppm 1.88 (2H, tt, J=7.4, 7.2 Hz), 2.19 (2H, t, J=7.4 Hz), 2.61 (2H, t, J=6.4 Hz), 3.08 (2H, t, J=7.2 Hz), 3.25 (2H, td, J=6.4, 5.7 Hz), 3.75 (2H, td, J=5.7, 5.3 Hz), 4.19 (2H, t, J=5.7 Hz), 4.86 (2H, q, J=8.7 Hz), 4.93 (1H, t, J=5.3 Hz), 6.43 (1H, d, J=3.4 Hz), 7.08 (1H, d, J=3.4 Hz), 7.18 (2H, d, J=8.8 Hz), 7.29 (2H, d, J=8.8 Hz), 8.22 (1H, t, J=5.7 Hz).

### Example 199

### N-(2-cyanoethyl)-2-{2-(ethylsulfanyl)-4-oxo-3-[4-(2,2,2-trifluoroethoxy)phenyl]-3,4-dihydro-7H-pyrrolo[2,3-d]pyrimidin-7-yl}acetamide

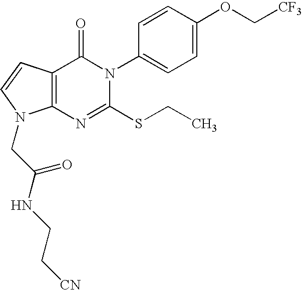

2-(Ethylsulfanyl)-3-[4-(2,2,2-trifluoroethoxy)phenyl]-3,7-dihydro-4H-pyrrolo[2,3-d]pyrimidin-4-one (100 mg) obtained by the method of Example 194, or a method pursuant to thereto, was dissolved in N,N-dimethylformamide (2 ml). Sodium hydride (60% in oil, 13 mg) was added to the solution under ice cooling, and the resulting mixture was stirred for 5 minutes. Then, 2-chloro-N-(2-cyanoethyl)acetamide (57 mg) obtained by the method of Reference Example 40, or a method pursuant to thereto was added thereto. The mixture was stirred for 1.5 hours under a nitrogen atmosphere. Acetic acid (100 μl) was added to the reaction mixture under ice cooling, and then the mixture was diluted with ethyl acetate (80 ml). The dilution was washed with water and saturated brine, dried over anhydrous magnesium sulfate, and then concentrated under reduced pressure. The resulting residue was purified by chromatography, and thus the title compound (99 mg) was obtained as a white solid.

¹H NMR (300 MHz, DMSO-d₆) δ ppm 1.23 (3H, t, J=7.3 Hz), 2.66 (2H, t, J=6.4 Hz), 3.03 (2H, q, J=7.3 Hz), 3.34 (2H, td, J=6.4, 5.7 Hz), 4.86 (2H, q, J=8.9 Hz), 4.83 (2H, s), 6.45 (1H, d, J=3.4 Hz), 7.04 (1H, d, J=3.4 Hz), 7.18 (2H, d, J=9.1 Hz), 7.28 (2H, d, J=9.1 Hz), 8.58 (1H, t, J=5.7 Hz).

### Example 200

### 7-(2-{[Tert-butyl(dimethyl)silyl]oxy}ethyl)-2-(ethylsulfanyl)-3-[4-(2,2,2-trifluoroethoxy)phenyl]-3,7-dihydro-4H-pyrrolo[2,3-d]pyrimidin-4-one

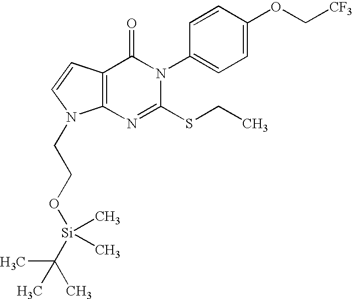

2-(Ethylsulfanyl)-3-[4-(2,2,2-trifluoroethoxy)phenyl]-3,7-dihydro-4H-pyrrolo[2,3-d]pyrimidin-4-one (100 mg) obtained by the method of Example 194, or a method pursuant to thereto was dissolved in N,N-dimethylformamide (2 ml). Sodium hydride (60% in oil, 13 mg) was added to the solution under ice cooling, and the resulting mixture was stirred for 5 minutes. Then, (2-bromoethoxy)(tert-butyl)dimethylsilane (87 μl) was added thereto. The mixture was stirred for 1.5 hours at room temperature under a nitrogen atmosphere. Acetic acid (100 μl) was added to the reaction mixture, and then the mixture was diluted with ethyl acetate (80 ml). The dilution was washed with water and saturated brine, dried over anhydrous magnesium sulfate, and then concentrated under reduced pressure. The resulting residue was purified by chromatography, and thus the title compound (123 mg) was obtained as white needle-shaped crystals.

¹H NMR (300 MHz, DMSO-d₆) δ ppm −0.08 (6 H, s), 0.79 (9 H, s), 1.28 (3H, t, J=7.3 Hz), 3.07 (2H, q, J=7.3 Hz), 3.92 (2H, t, J=5.5 Hz), 4.23 (2H, t, J=5.5 Hz), 4.86 (2H, q, J=8.9 Hz), 6.44 (1H, d, J=3.4 Hz), 7.08 (1H, d, J=3.4 Hz), 7.18 (2H, d, J=9.1 Hz), 7.25 (2H, d, J=9.1 Hz).

### Example 201

### 2-(Ethylsulfanyl)-7-(2-hydroxyethyl)-3-[4-(2,2,2-trifluoroethoxy)phenyl]-3,7-dihydro-4H-pyrrolo[2,3-d]pyrimidin-4-one

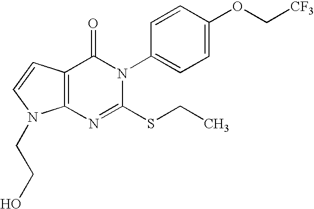

A mixture of 7-(2-{[tert-butyl(dimethyl)silyl]oxy}ethyl)-2-(ethylsulfanyl)-3-[4-(2,2,2-trifluoroethoxy)phenyl]-3,7-dihydro-4H-pyrrolo[2,3-d]pyrimidin-4-one (117 mg) obtained in Example 200, tetrabutylammonium fluoride (1 M tetrahydrofuran solution, 266 μl) and tetrahydrofuran (3 ml) was stirred for one hour at room temperature. Acetic acid (100 μl) was added to the reaction mixture solution, and the resultant was concentrated under reduced pressure and then was azeotropically boiled with toluene. The resulting residue was purified by chromatography, and thus the title compound (79 mg) was obtained as a white solid.

¹H NMR (400 MHz, DMSO-d₆) δ ppm 1.27 (3H, t, J=7.3 Hz), 3.07 (2H, q, J=7.3 Hz), 3.76 (2H, td, J=5.7, 5.3 Hz), 4.18 (2H, t, J=5.7 Hz), 4.87 (2H, q, J=8.8 Hz), 4.95 (1H, t, J=5.3 Hz), 6.43 (1H, d, J=3.4 Hz), 7.09 (1H, d, J=3.4 Hz), 7.18 (2H, d, J=8.8 Hz), 7.29 (2H, d, J=8.8 Hz).

### Example 202

### 2-(Ethylsulfanyl)-3-[4-(2,2,2-trifluoroethoxy)phenyl]-7-(2,2,2-trifluoroethyl)-3,7-dihydro-4H-pyrrolo[2,3-d]primidin-4-one

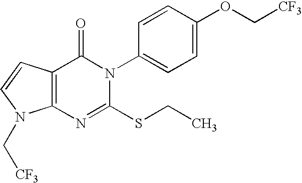

2-(Ethylsulfanyl)-3-[4-(2,2,2-trifluoroethoxy)phenyl]-3,7-dihydro-4H-pyrrolo[2,3-d]pyrimidin-4-one (100 mg) obtained by the method of Example 194, or a method pursuant to thereto was dissolved in N,N-dimethylformamide (2 ml), and sodium hydride (60% in oil, 13 mg) was added to the solution under ice cooling. The mixture was stirred for 5 minutes. Then, 1,1,1-trifluoro-2-iodoethane (267 μl) was added thereto, and the resulting mixture was stirred for 2 days at room temperature under a nitrogen atmosphere. Acetic acid (100 μl) was added to the reaction mixture, and then the mixture was diluted with ethyl acetate (80 ml). The dilution was washed with water and saturated brine, dried over anhydrous magnesium sulfate, and then concentrated under reduced pressure. The resulting residue was purified by chromatography, and thus the title compound (57 mg) was obtained as a white solid.

¹H NMR (300 MHz, DMSO-d₆) δ ppm 1.26 (3H, t, J=7.3 Hz), 3.09 (2H, q, J=7.3 Hz), 4.87 (2H, q, J=8.9 Hz), 5.09 (2H, q, J=9.2 Hz), 6.57 (1H, d, J=3.6 Hz), 7.15 (1H, d, J=3.6 Hz), 7.19 (2H, d, J=9.0 Hz), 7.33 (2H, d, J=9.0 Hz).

### Example 203

### 7-(Cyclopropylmethyl)-2-(ethylsulfanyl)-3-[4-(2,2,2-trifluoroethoxy)phenyl]-3,7-dihydro-4H-pyrrolo[2,3-d]pyrimidin-4-one

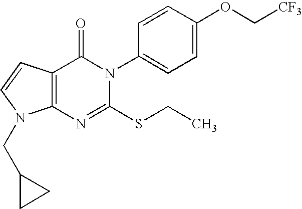

2-(Ethylsulfanyl)-3-[4-(2,2,2-trifluoroethoxy)phenyl]-3,7-dihydro-4H-pyrrolo[2,3-d]pyrimidin-4-one (100 mg) obtained by the method of Example 194, or a method pursuant to thereto, and (bromomethyl)cyclopropane (132 μl) were dissolved in N,N-dimethylformamide (2 ml), and sodium hydride (60% in oil, 13 mg) was added to the solution under ice cooling. Then, the mixture was stirred for one hour at room temperature under a nitrogen atmosphere. A saturated aqueous solution of ammonium chloride (3 ml) was added to the reaction solution under ice cooling, and then the mixture was diluted with ethyl acetate (80 ml). The dilution was washed with water and saturated brine, dried over anhydrous magnesium sulfate, and then concentrated under reduced pressure. The resulting residue was purified by chromatography, and thus the title compound (114 mg) was obtained as white needle-shaped crystals.

¹H NMR (400 MHz, DMSO-d₆) δ ppm 0.38-0.46 (2H, m), 0.49-0.60 (2H, m), 1.22-1.33 (1H, m), 1.27 (3H, t, J=7.3 Hz), 3.07 (2H, q, J=7.3 Hz), 3.99 (2H, d, J=7.1 Hz), 4.87 (2H, q, J=8.9 Hz), 6.45 (1H, d, J=3.4 Hz), 7.16 (1H, d, J=3.4 Hz), 7.18 (2H, d, J=8.8 Hz), 7.30 (2H, d, J=8.8 Hz).

### Example 204

### 2-(Ethylsulfanyl)-7-(methoxymethyl)-3-[4-(2,2,2-trifluoroethoxy)phenyl]-3,7-dihydro-4H-pyrrolo[2,3-d]pyrimidin-4-one

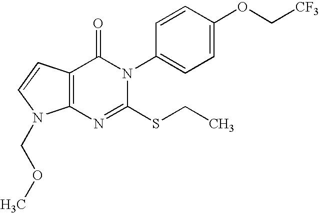

2-(Ethylsulfanyl)-3-[4-(2,2,2-trifluoroethoxy)phenyl]-3,7-dihydro-4H-pyrrolo[2,3-d]pyrimidin-4-one (100 mg) obtained by the method of Example 194, or a method pursuant to thereto, and chloromethylmethyl ether (103 μl) were dissolved in N,N-dimethylformamide (2 ml), and sodium hydride (60% in oil, 13 mg) was added to the solution under ice cooling. Then, the mixture was stirred for one hour at room temperature under a nitrogen atmosphere. A saturated aqueous solution of ammonium chloride (3 ml) was added to the reaction solution under ice cooling, and then the mixture was diluted with ethyl acetate (80 ml). The dilution was washed with water and saturated brine, dried over anhydrous magnesium sulfate, and then concentrated under reduced pressure. The resulting residue was purified by chromatography, and thus the title compound (104 mg) was obtained as white needle-shaped crystals.

¹H NMR (400 MHz, DMSO-d₆) δ ppm 1.27 (3H, t, J=7.3 Hz), 3.08 (2H, q, J=7.3 Hz), 3.28 (3H, s), 4.87 (2H, q, J=8.8 Hz), 5.47 (2H, s), 6.52 (1H, d, J=3.4 Hz), 7.19 (2H, d, J=8.8 Hz), 7.20 (1H, d, J=3.4 Hz), 7.32 (2H, d, J=8.8 Hz).

### Example 205

### N-(2-cyanoethyl)-2-({4-oxo-3-[4-(2,2,2-trifluoroethoxy)phenyl]-4,7-dihydro-3H-pyrrolo[2,3-d]pyrimidin-2-yl}sulfanyl)acetamide

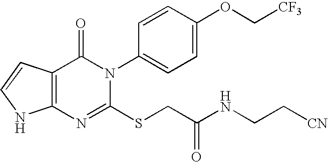

A mixture of 2-thioxo-3-[4-(2,2,2-trifluoroethoxy)phenyl]-1,2,3,7-tetrahydro-4H-pyrrolo[2,3-d]pyrimidin-4-one (150 mg) obtained by the method of Example 26, or a method pursuant to thereto, 2-chloro-N-(2-cyanoethyl)acetamide (84 mg) obtained by the method of Reference Example 40, or a method pursuant to thereto, triethylamine (246 μl) and acetonitrile (3 ml) was heated to reflux for one hour. The reaction mixture was returned to room temperature, and then was diluted with ethyl acetate (80 m1). The dilution was washed with water and saturated brine, dried over anhydrous magnesium sulfate, and then concentrated under reduced pressure. The resulting residue was purified by chromatography, and thus the title compound (182 mg) was obtained as white needle-shaped crystals.

¹H NMR (300 MHz, DMSO-d₆) δ ppm 2.62 (2H, t, J=6.6 Hz), 3.30 (2H, td, J=6.6, 5.7 Hz), 3.81 (2H, s), 4.87 (2H, q, J=8.8 Hz), 6.44 (1H, d, J=3.4 Hz), 6.99 (1H, d, J=3.4 Hz), 7.20 (2H, d, J=9.1 Hz), 7.33 (2H, d, J=9.1 Hz), 8.41 (1H, t, J=5.7 Hz), 11.76 (1H, s).

### Example 206

### 2-(Ethylsulfinyl)-3-[4-(2,2,2-trifluoroethoxy)phenyl]-3,7-dihydro-4H-pyrrolo[2,3-d]pyrimidin-4-one

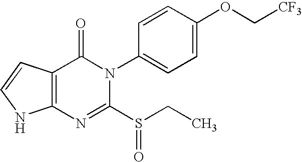

A mixture of 2-(ethylsulfanyl)-3-[4-(2,2,2-trifluoroethoxy)phenyl]-3,7-dihydro-4H-pyrrolo[2,3-d]pyrimidin-4-one (400 mg) obtained by the method of Example 194, or a method pursuant to thereto, Oxone (registered trademark) monopersulfate compound (1.5 g), methanol (30 ml) and water (15 ml) was stirred for 2 days at room temperature, and then was concentrated under reduced pressure. Water was added to the residue, and then the mixture was extracted with ethyl acetate. The organic layer was washed with water and saturated brine, dried over anhydrous sodium sulfate, and then concentrated under reduced pressure. The residue was purified by chromatography, and then was recrystallized from ethyl acetate. Thus, the title compound (124 mg) was obtained as a white solid. Furthermore, 2-(ethylsulfonyl)-3-[4-(2,2,2-trifluoroethoxy)phenyl]-3,7-dihydro-4H-pyrrolo[2,3-d]pyrimidin-4-one (81 mg) of Example 207 was also obtained as a white solid.

¹H NMR (300 MHz, DMSO-d₆) δ ppm 1.06 (3H, t, J=7.4 Hz), 2.72-2.86 (1H, m), 2.91-3.06 (1H, m), 4.87 (2H, q, J=8.9 Hz), 6.59 (1H, dd, J=3.2, 2.1 Hz), 7.18-7.24 (2H, m), 7.26 (1H, dd, J=3.3, 2.5 Hz), 7.40-7.46 (1H, m), 7.50-7.53 (1H, m), 12.42 (1H, br. s.).

### Example 207

### 2-(Ethylsulfonyl)-3-[4-(2,2,2-trifluoroethoxy)phenyl]-3,7-dihydro-4H-pyrrolo[2,3-d]pyrimidin-4-one

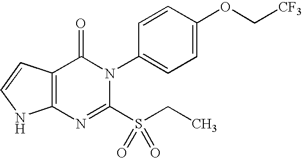

A mixture of 2-(ethylsulfanyl)-3-[4-(2,2,2-trifluoroethoxy)phenyl]-3,7-dihydro-4H-pyrrolo[2,3-d]pyrimidin-4-one (400 mg) obtained by the method of Example 194, or a method pursuant to thereto, Oxone (registered trademark) monopersulfate compound (1.5 g), methanol (30 ml) and water (15 ml) was stirred for 2 days at room temperature, and then was concentrated under reduced pressure. Water was added to the residue, and then the mixture was extracted with ethyl acetate. The organic layer was washed with water and saturated brine, dried over anhydrous sodium sulfate, and then concentrated under reduced pressure. The residue was purified by chromatography, and then was recrystallized from ethyl acetate. Thus, the title compound (81 mg) was obtained as a white solid. Furthermore, 2-(ethylsulfinyl)-3-[4-(2,2,2-trifluoroethoxy)phenyl]-3,7-dihydro-4H-pyrrolo[2,3-d]pyrimidin-4-one (124 mg) of Example 206 was also obtained as a white solid.

¹H NMR (300 MHz, DMSO-d₆) δ ppm 1.29 (3H, t, J=7.3 Hz), 3.58 (2H, q, J=7.3 Hz), 4.85 (2H, q, J=8.9 Hz), 6.64 (1H, dd, J=3.2, 2.1 Hz), 7.13 (2H, d, J=9.0 Hz), 7.31-7.37 (3H, m), 12.56 (1H, br. s.).

### Example 208

### 3-[4-(Cyclopropylmethoxy)phenyl]-2-(ethylsulfanyl)-3,7-dihydro-4H-pyrrolo[2,3-d]primidin-4-one

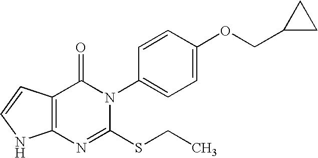

A 1 M aqueous solution of sodium hydrogen carbonate (1.0 ml) was added to a mixture of 3-[4-(cyclopropylmethoxy)phenyl]-2-thioxo-1,2,3,7-tetrahydro-4H-pyrrolo[2,3-d]pyrimidin-4-one (313 mg) obtained by the method of Example 31, or a method pursuant to thereto, iodoethane (80 μl) and N,N-dimethylformamide (10 ml), and the resulting mixture was stirred for 2 hours at 70° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (196 mg) was obtained as a white powder.

¹H NMR (300 MHz, DMSO-d₆) δ ppm 0.31-0.38 (2H, m), 0.56-0.64 (2H, m), 1.20-1.29 (4H, m), 3.03 (2H, q, J=7.3 Hz), 3.88 (2H, d, J=6.8 Hz), 6.42 (1H, d, J=3.4 Hz), 6.96 (1H, d, J=3.4 Hz), 7.02 (2H, d, J=9.1 Hz), 7.19 (2H, d, J=9.0 Hz), 11.84 (1H, br. s.).

### Example 209

### 2-Ethoxy-3-[4-(2,2,2-trifluoroethoxy)phenyl]-3,7-dihydro-4H-pyrrolo[2,3-d]pyrimidin-4-one

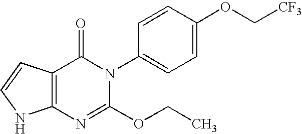

A mixture of 2-(ethylsulfonyl)-3-[4-(2,2,2-trifluoroethoxy)phenyl]-3,7-dihydro-4H-pyrrolo[2,3-d]pyrimidin-4-one (200 mg) obtained by the method of Example 207, or a method pursuant to thereto, a 20% sodium ethoxide-ethanol solution (1 ml), ethanol (20 ml) and tetrahydrofuran (20 ml) was stirred for one hour at 60° C., and then was concentrated under reduced pressure. Water and ethyl acetate were added to the residue, and the pH of the mixture was adjusted to about 6 with a 5% aqueous solution of citric acid. Then, the mixture was extracted with ethyl acetate. The organic layer was washed with water and saturated brine, dried over anhydrous sodium sulfate, and then concentrated under reduced pressure. The residue was purified by chromatography, and then was recrystallized from a mixed solvent of ethyl acetate/diisopropyl ether. Thus, the title compound (74 mg) was obtained as a white solid.

¹H NMR (300 MHz, DMSO-d₆) δ ppm 1.15 (3H, t, J=7.1 Hz), 4.30 (2H, q, J=7.1 Hz), 4.83 (2H, q, J=8.8 Hz), 6.37 (1H, dd, J=3.4, 1.9 Hz), 6.88 (1H, dd, J=3.0, 2.3 Hz), 7.13 (2H, d, J=8.7 Hz), 7.23 (2H, d, J=8.7 Hz), 11.68 (1H, br. s.).

### Example 210

### 2-[(2-{[Tert-butyl(dimethyl)silyl]oxy}ethyl)sulfanyl]-3-[4-(2,2,2-trifluoroethoxy)phenyl]-3,7-dihydro-4H-pyrrolo[2,3-d]pyrimidin-4-one

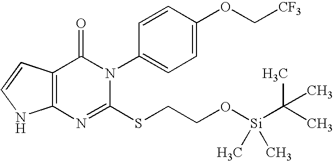

A mixture of 2-thioxo-3-[4-(2,2,2-trifluoroethoxy)phenyl]-1,2,3,7-tetrahydro-4H-pyrrolo[2,3-d]pyrimidin-4-one (171 mg) obtained by the method of Example 26, or a method pursuant to thereto, a 1 M aqueous solution of sodium hydrogen carbonate (0.5 ml), (2-bromoethoxy)(tert-butyl)dimethylsilane (129 μl) and N,N-dimethylformamide (5 ml) was heated to 100° C. and was stirred for one hour. The reaction mixture was returned to room temperature, and then was diluted with ethyl acetate (80 ml). The dilution was washed with water and saturated brine, dried over anhydrous magnesium sulfate, and then concentrated under reduced pressure. The resulting residue was purified by chromatography, and thus the title compound (240 mg) was obtained as white crystals.

¹H NMR (300 MHz, DMSO-d₆) δ ppm 0.01 (6 H, s), 0.82 (9 H, s), 3.20 (2H, t, J=6.4 Hz), 3.78 (2H, t, J=6.4 Hz), 4.86 (2H, q, J=8.9 Hz), 6.43 (1H, d, J=3.2 Hz), 6.98 (1H, dd, J=3.2, 1.2 Hz), 7.18 (2H, d, J=9.1 Hz), 7.28 (2H, d, J=9.1 Hz), 11.80 (1H, br.

s.).

### Example 211

### 7-Ethyl-2-[(2-hydroxyethyl)sulfanyl]-3-[4-(2,2,2-trifluoroethoxy)phenyl]-3,7-dihydro-4H-pyrrolo[2,3-d]pyrimidin-4-one

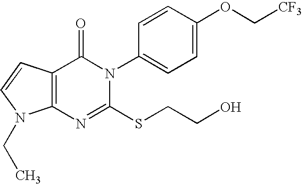

2-[(2-{[Tert-butyl(dimethyl)silyl]oxy}ethyl)sulfanyl]-3-[4-(2,2,2-trifluoroethoxy)phenyl]-3,7-dihydro-4H-pyrrolo[2,3-d]pyrimidin-4-one (230 mg) obtained by the method of Example 210, or a method pursuant to thereto, and iodoethane (186 μl) were dissolved in N,N-dimethylformamide (5 ml), and sodium hydride (60% in oil, 27.6 mg) was added to the solution under ice cooling. Subsequently, the mixture was stirred for one hour at 0° C. under a nitrogen atmosphere. Under ice cooling, a saturated aqueous solution of ammonium chloride (5 ml) was added, and then water (5 ml) was added to the reaction solution. The mixture was extracted with ethyl acetate (80 ml), washed with water and saturated brine, dried over anhydrous magnesium sulfate, and then concentrated under reduced pressure, to obtain a light yellow oily substance. This oily substance was dissolved in tetrahydrofuran (5 ml), and tetrabutylammonium fluoride (1 M tetrahydrofuran solution, 0.6 ml) was added to the solution. The resulting mixture was stirred for 10 minutes at room temperature. A saturated aqueous solution of ammonium chloride (5 ml) and water (2 ml) were added to the reaction solution, and then the mixture was extracted with ethyl acetate. The extract was washed with saturated brine, dried over anhydrous magnesium sulfate, and then concentrated under reduced pressure. The resulting residue was purified by chromatography, and thus the title compound (180 mg) was obtained as a white solid.

¹H NMR (300 MHz, DMSO-d₆) δ ppm 1.39 (3H, t, J=7.2 Hz), 3.19 (2H, t, J=6.4 Hz), 3.64 (2H, td, J=6.4, 5.7 Hz), 4.16 (2H, q, J=7.2 Hz), 4.86 (2H, q, J=8.8 Hz), 4.92 (1H, t, J=5.7 Hz), 6.44 (1H, d, J=3.4 Hz), 7.11 (1H, d, J=3.4 Hz), 7.18 (2H, d, J=9.0 Hz), 7.30 (2H, d, J=9.0 Hz).

### Example 212

### 2-(Methylsulfanyl)-3-[4-(2,2,2-trifluoroethoxy)phenyl]-3,7-dihydro-4H-pyrrolo[2,3-d]pyrimidin-4-one

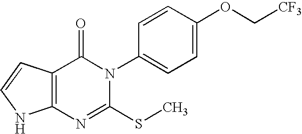

2-Thioxo-3-[4-(2,2,2-trifluoroethoxy)phenyl]-1,2,3,7-tetrahydro-4H-pyrrolo[2,3-d]pyrimidin-4-one (300 mg) obtained by the method of Example 26 or a method pursuant to thereto was dissolved in N,N-dimethylformamide (5 ml). A 1 M aqueous solution of sodium hydrogen carbonate (880 μl) and iodomethane (275 μl) were added to the solution at room temperature. Then the resulting solution was heated to 100° C., and was stirred for 30 minutes. The reaction mixture was cooled to room temperature and diluted with ethyl acetate (80 ml). The mixture was washed with water (15 ml) three times, washed with saturated brine, dried over anhydrous magnesium sulfate and concentrated under reduced pressure. The resulting brown solid was purified by chromatography and recrystallized from a mixed solvent of ethyl acetate/hexane to give the title compound (186 mg) as white crystals.

¹H NMR (400 MHz, DMSO-d₆) δ ppm 2.42 (3H, s), 4.87 (2H, q, J=8.9 Hz), 6.43 (1H, d, J=3.4 Hz), 6.98 (1H, d, J=3.4 Hz), 7.19 (2H, d, J=9.0 Hz), 7.31 (2H, d, J=9.0 Hz), 11.90 (1H, s).

### Example 213

### 2-[(3-Ethoxypropyl)sulfanyl]-3-[4-(2,2,2-trifluoroethoxy)phenyl]-3,7-dihydro-4H-pyrrolo[2,3-d]pyrimidin-4-one

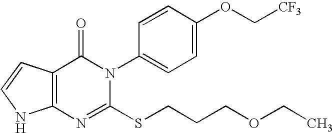

A mixture of 2-thioxo-3-[4-(2,2,2-trifluoroethoxy)phenyl]-1,2,3,7-tetrahydro-4H-pyrrolo[2,3-d]pyrimidin-4-one (200 mg) obtained by the method of Example 26, or a method pursuant to thereto, a 1 M aqueous solution of sodium hydrogen carbonate (586 μl), 3-ethoxypropyl 4-methylbenzenesulfonate (167 mg) obtained by the method described in a published document, Canadian Journal of Chemistry (Can. J. Chem.), Vol. 33, p. 1207 (1955), or a method pursuant to thereto, and N,N-dimethylformamide (3 ml) was heated to 100° C., and then was stirred for 30 minutes. The reaction mixture was returned to room temperature, and then was diluted with ethyl acetate (80 ml). The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (165 mg) was obtained as white crystals.

¹H NMR (300 MHz, DMSO-d₆) δ ppm 1.07 (3H, t, J=6.9 Hz), 1.83 (2H, tt, J=7.2, 6.2 Hz), 3.08 (2H, t, J=7.2 Hz), 3.38 (2H, q, J=6.9 Hz), 3.39 (2H, t, J=6.2 Hz), 4.86 (2H, q, J=8.9 Hz), 6.42 (1H, d, J=3.4 Hz), 6.97 (1H, d, J=3.4 Hz), 7.18 (2H, d, J=9.1 Hz), 7.30 (2H, d, J=9.1 Hz), 11.85 (1H, s).

### Example 214

### 2-[(2-Hydroxyethyl)sulfanyl]-3-[4-(2,2,2-trifluoroethoxy)phenyl]-3,7-dihydro-4H-pyrrolo[2,3-d]pyrimidin-4-one

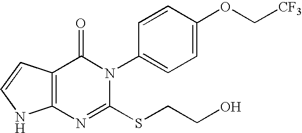

2-[(2-{[Tert-butyl(dimethyl)silyl]oxy}ethyl)sulfanyl]-3-[4-(2,2,2-trifluoroethoxy)phenyl]-3,7-dihydro-4H-pyrrolo[2,3-d]pyrimidin-4-one (289 mg) obtained by the method of Example 210, or a method pursuant to thereto was dissolved in tetrahydrofuran (5 ml), and tetrabutylammonium fluoride (1 M tetrahydrofuran solution, 752 μl) was added to the solution. The resulting mixture was stirred for 30 minutes at room temperature. A saturated aqueous solution of ammonium chloride (5 ml) and water (2 ml) were added to the reaction solution, and then the mixture was extracted with ethyl acetate. The extract was washed with saturated brine, dried over anhydrous magnesium sulfate, and then concentrated under reduced pressure. The resulting residue was purified by chromatography, and thus the title compound (168 mg) was obtained as a white solid.

¹H NMR (300 MHz, DMSO-d₆) δ ppm 3.17 (2H, t, J=6.4 Hz), 3.61 (2H, td, J=6.4, 5.4 Hz), 4.86 (2H, q, J=8.9 Hz), 4.91 (1H, t, J=5.4 Hz), 6.42 (1H, d, J=3.4 Hz), 6.97 (1H, d, J=3.4 Hz), 7.18 (2H, d, J=9.1 Hz), 7.30 (2H, d, J=9.1 Hz), 11.85 (1H, br. s.).

### Example 215

### 3-[4-(Cyclopropylmethoxy)phenyl]-2-{[3-(methylsulfonyl)propyl]sulfanyl}-3,7-dihydro-4H-pyrrolo[2,3-d]pyrimidin-4-one

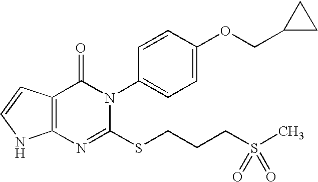

A 1 M aqueous solution of sodium hydrogen carbonate (0.64 ml) was added to a mixture of 3-[4-(cyclopropylmethoxy)phenyl]-2-thioxo-1,2,3, 7-tetrahydro-4H-pyrrolo[2,3-d]pyrimidin-4-one (200 mg) obtained by the method of Example 31, or a method pursuant to thereto, 3-(methylsulfonyl)propyl 4-methylbenzenesulfonate (187 mg) obtained by the method described in a published document, WO 08/1931, or a method pursuant to thereto, sodium iodide (96 mg) and N,N-dimethylformamide (10 ml), and the resulting mixture was stirred for 15 hours at 100° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (160 mg) was obtained as a white powder.

¹H NMR (300 MHz, DMSO-d₆) δ ppm 0.32-0.39 (2H, m), 0.57-0.64 (2H, m), 1.21-1.32 (1H, m), 2.01-2.13 (2H, m), 2.97 (3H, s), 3.12-3.21 (4H, m), 3.88(2H, d, J=6.8 Hz), 6.43 (1H, d, J=3.4 Hz), 6.98 (1H, d, J=3.4 Hz), 7.04 (2H, d, J=9.1 Hz), 7.22 (2H, d, J=9.1 Hz), 11.84 (1H, s).

### Example 216

### 2-Methoxy-3-[4-(2,2,2-trifluoroethoxy)phenyl]-3,7-dihydro-4H-pyrrolo[2,3-d]pyrimidin-4-one

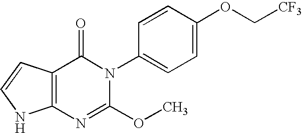

A mixture of 2-(ethylsulfonyl)-3-[4-(2,2,2-trifluoroethoxy)phenyl]-3,7-dihydro-4H-pyrrolo[2,3-d]pyrimidin-4-one (200 mg) obtained by the method of Example 207, or a method pursuant to thereto, a 28% sodium methoxide-methanol solution (1 ml), methanol (20 ml) and tetrahydrofuran (20 ml) was stirred for one hour at 60° C., and then was concentrated under reduced pressure. Water and ethyl acetate were added to the residue, and the pH of the mixture was adjusted to about 6 with a 5% aqueous solution of citric acid. Then, the mixture was extracted with ethyl acetate. The organic layer was washed with water and saturated brine, dried over anhydrous sodium sulfate, and then concentrated under reduced pressure. The residue was purified by chromatography, and then was recrystallized from a mixed solvent of ethyl acetate/diisopropyl ether. Thus, the title compound (126 mg) was obtained as a white solid.

¹H NMR (300 MHz, DMSO-d₆) δ ppm 3.81 (3H, s), 4.83 (2H, q, J=8.7 Hz), 6.38 (1H, d, J=3.4 Hz), 6.89 (1H, d, J=3.4 Hz), 7.13 (2H, d, J=8.9 Hz), 7.24 (2H, d, J=8.9 Hz), 11.71 (1H, s).

### Example 217

### 2-Propoxy-3-[4-(2,2,2-trifluoroethoxy)phenyl]-3,7-dihydro-4H-pyrrolo[2,3-d]pyrimidin-4-one

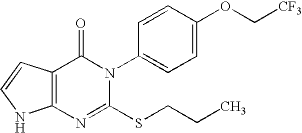

Propan-1-ol (10 ml) was added dropwise to a mixture of sodium hydride (60% in oil, 200 mg) and tetrahydrofuran (20 ml) in an ice water bath. To the mixture, 2-(ethylsulfonyl)-3-[4-(2,2,2-trifluoroethoxy)phenyl]-3,7-dihydro-4H-pyrrolo[2,3-d]pyrimidin-4-one (200 mg) obtained by the method of Example 207, or a method pursuant to thereto was added, and the resulting mixture was stirred for one hour at 60° C., and then was concentrated under reduced pressure. Water was added to the residue, and the pH of the mixture was adjusted to about 6 with a 5% aqueous solution of citric acid. Then, the mixture was extracted with ethyl acetate. The organic layer was washed with water and saturated brine, dried over anhydrous sodium sulfate, and then concentrated under reduced pressure. The residue was purified by chromatography, and then was recrystallized from a mixed solvent of ethyl acetate/diisopropyl ether. Thus, the title compound (136 mg) was obtained as a brown solid.

¹H NMR (300 MHz, DMSO-d₆) δ ppm 0.74 (3H, t, J=7.4 Hz), 1.46-1.62 (2H, m), 4.20 (2H, t, J=6.4 Hz), 4.83 (2H, q, J=8.7 Hz), 6.37 (1H, dd, J=3.2, 2.1 Hz), 6.88 (1H, dd, J=3.2, 2.5 Hz), 7.14 (2H, d, J=8.9 Hz), 7.23 (2H, d, J=8.9 Hz), 11.67 (1H, br. s.).

### Example 218

### 3-[4-(2,2,2-Trifluoroethoxy)phenyl]-1H-pyrrolo[2,3-d]pyrimidine-2,4(3H,7H)-dione

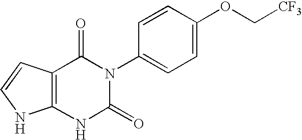

A mixture of 2-(ethylsulfonyl)-3-[4-(2,2,2-trifluoroethoxy)phenyl]-3,7-dihydro-4H-pyrrolo[2,3-d]pyrimidin-4-one (200 mg) obtained by the method of Example 207, or a method pursuant to thereto, a 1 M aqueous solution of sodium hydroxide (2 ml) and tetrahydrofuran (20 ml) was stirred for 3 days at 30° C., and then was concentrated under reduced pressure. Water and ethyl acetate were added to the residue, and the pH of the mixture was adjusted to about 6 with a 5% aqueous solution of citric acid. Then, the mixture was extracted with ethyl acetate. The organic layer was washed with water and saturated brine, dried over anhydrous sodium sulfate, and then concentrated under reduced pressure. The residue was purified by chromatography, and then was recrystallized from ethyl acetate. Thus, the title compound (83 mg) was obtained as a white solid.

¹H NMR (300 MHz, DMSO-d₆) δ ppm 4.81 (2H, q, J=9.0 Hz), 6.29 (1H, d, J=3.4 Hz), 6.63 (1H, d, J=3.4 Hz), 7.10 (2H, d, J=9.1 Hz), 7.17 (2H, d, J=9.1 Hz), 11.24 (1H, br. s.), 11.87(1H, br. s.).

### Example 219

### 2-{[2-Hydroxy-3-(tetrahydro-2H-pyran-4-yloxy)propyl]sulfanyl}-3-[4-(2,2,2-trifluoroethoxy)phenyl]-3,7-dihydro-4H-pyrrolo[2,3-d]pyrimidin-4-one

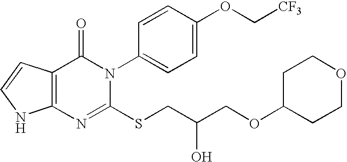

A 1 M aqueous solution of sodium hydrogen carbonate (0.59 ml) was added to a mixture of 2-thioxo-3-[4-(2,2,2-trifluoroethoxy)phenyl]-1,2,3,7-tetrahydro-4H-pyrrolo[2,3-d]pyrimidin-4-one (200 mg) obtained by the method of Example 26, or a method pursuant to thereto, 4-(oxiran-2-yl methoxy)tetrahydro-2H-pyrane (93 mg) obtained in Reference Example 66, sodium iodide (88 mg) and N,N-dimethylformamide (10 ml), and the resulting mixture was stirred for 15 hours at 100° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (83.1 mg) was obtained as a white powder.

¹H NMR (300 MHz, DMSO-d₆) δ ppm 1.27-1.43 (2H, m), 1.71-1.85 (2H, m), 3.05-3.50 (8 H, m), 3.70-3.84 (3H, m), 4.86 (2H, q, J=9.0 Hz), 6.42 (1H, dd, J=3.2, 2.3 Hz), 6.97 (1H, dd, J=3.2, 2.3 Hz), 7.18 (2H, d, J=9.0 Hz), 7.30 (2H, d, J=9.0 Hz), 11.82 (1H, br. s.).

### Example 220

### 2-(Methylsulfonyl)-3-[4-(2,2,2-trifluoroethoxy)phenyl]-3,7-dihydro-4H-pyrrolo[2,3-d]pyrimidin-4-one

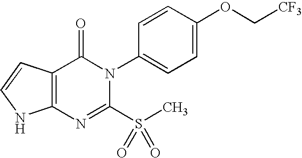

A mixture of 2-(methylsulfanyl)-3-[4-(2,2,2-trifluoroethoxy)phenyl]-3,7-dihydro-4H-pyrrolo[2,3-d]pyrimidin-4-one (2.96 g) obtained by the method of Example 212, or a method pursuant to thereto, 3-chlorobenzoic acid (70%, 5.13 g) and ethyl acetate (300 ml) was stirred for 2 hours at 60° C., and then was cooled to room temperature. A saturated aqueous solution of sodium thiosulfate was added, and then the mixture was extracted with ethyl acetate. The organic layer was washed with a 5% aqueous solution of sodium hydrogen carbonate, water and saturated brine, and then dried over anhydrous sodium sulfate. The residue was purified by chromatography to obtain a brown solid (2.98 g), and this solid (150 mg) was recrystallized from a mixed solvent of ethyl acetate/hexane. Thus, the title compound (64 mg) was obtained as a pale brown solid.

¹H NMR (300 MHz, DMSO-d₆) δ ppm 3.39 (3H, s), 4.85 (2H, q, J=8.9 Hz), 6.64 (1H, d, J=3.4 Hz), 7.14 (2H, d, J=9.0 Hz), 7.32 (2H, d, J=9.0 Hz), 7.36 (1H, d, J=3.4 Hz), 11.89 (1H, br. s.).

### Example 221

### 2-(1-Methylethoxy)-3-[4-(2,2,2-trifluoroethoxy)phenyl]-3,7-dihydro-4H-pyrrolo[2,3-d]pyrimidin-4-one

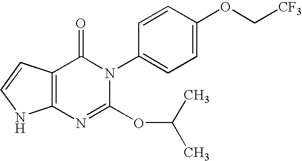

Propan-2-ol (5 ml) was added dropwise to a mixture of sodium hydride (60% in oil, 100 mg) and tetrahydrofuran (10 ml). To the mixture, 2-(methylsulfonyl)-3-[4-(2,2,2-trifluoroethoxy)phenyl]-3,7-dihydro-4H-pyrrolo[2,3-d]pyrimidin-4-one (200 mg) obtained by the method of Example 220, or a method pursuant to thereto was added, and the resulting mixture was stirred for 2 hours at 60° C., and then was concentrated under reduced pressure. Water was added to the residue, and the pH of the mixture was adjusted to about 6 with a 5% aqueous solution of citric acid. Then, the mixture was extracted with ethyl acetate. The organic layer was washed with water and saturated brine, dried over anhydrous sodium sulfate, and then concentrated under reduced pressure. The residue was purified by chromatography, and then was recrystallized from a mixed solvent of ethyl acetate/hexane. Thus, the title compound (104 mg) was obtained as a white solid.

¹H NMR (300 MHz, DMSO-d₆) δ ppm 1.17 (6 H, d, J=6.2 Hz), 4.83 (2H, q, J=8.9 Hz), 5.17 (1H, spt, J=6.2 Hz), 6.37 (1H, d, J=3.3 Hz), 6.87 (1H, d, J=3.3 Hz), 7.12 (2H, d, J=9.0 Hz), 7.20 (2H, d, J=9.0 Hz), 11.66 (1H, s).

### Example 222

### 2-Butoxy-3-[4-(2,2,2-trifluoroethoxy)phenyl]-3,7-dihydro-4H-pyrrolo[2,3-d]pyrimidin-4-one

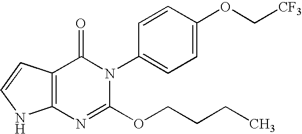

1-Butanol (5 ml) was added dropwise to a mixture of sodium hydride (60% in oil, 100 mg) and tetrahydrofuran (10 ml). To the mixture, 2-(methyl sulfonyl)-3-[4-(2,2,2-trifluoroethoxy)phenyl]-3,7-dihydro-4H-pyrrolo[2,3-d]pyrimidin-4-one (200 mg) obtained by the method of Example 220, or a method pursuant to thereto was added, and the resulting mixture was stirred for 2 hours at 60° C., and then was concentrated under reduced pressure. Water was added to the residue, and the pH of the mixture was adjusted to about 6 with a 5% aqueous solution of citric acid. Then, the mixture was extracted with ethyl acetate. The organic layer was washed with water and saturated brine, dried over anhydrous sodium sulfate, and then concentrated under reduced pressure. The residue was purified by chromatography, and then was recrystallized from a mixed solvent of ethyl acetate/hexane. Thus, the title compound (94 mg) was obtained as a white solid.

¹H NMR (300 MHz, DMSO-d₆) δ ppm 0.79 (3H, t, J=7.3 Hz), 1.10-1.25 (2H, m), 1.44-1.56 (2H, m), 4.24 (2H, t, J=6.4 Hz), 4.83 (2H, q, J=9.0 Hz), 6.37 (1H, d, J=3.4 Hz), 6.88 (1H, d, J=3.4 Hz), 7.13 (2H, d, J=9.1 Hz), 7.22 (2H, d, J=9.1 Hz), 11.68 (1H, s).

### Example 223

### 2-[(Difluoromethyl)sulfanyl]-3-[4-(2,2,2-trifluoroethoxy)phenyl]-3,7-dihydro-4H-pyrrolo[2,3-d]pyrimidin-4-one

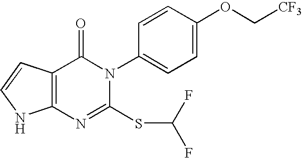

Difluoro(iodo)methane was blown into N,N-dimethylformamide (5 ml) at room temperature for 15 minutes, and then 2-thioxo-3-[4-(2,2,2-trifluoroethoxy)phenyl]-1,2,3,7-tetrahydro-4H-pyrrolo[2,3-d]pyrimidin-4-one (200 mg) obtained by the method of Example 26, or a method pursuant to thereto, and a 1 M aqueous solution of sodium hydrogen carbonate (0.59 ml) were added thereto. The mixture was stirred for 15 hours at 50° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (108 mg) was obtained as a white powder.

¹H NMR (300 MHz, DMSO-d₆) δ ppm 4.89 (2H, q, J=8.7 Hz), 6.50 (1H, d, J=3.2 Hz), 7.07 (1H, d, J=3.2 Hz), 7.22 (2H, d, J=8.9 Hz), 7.41 (2H, d, J=8.9 Hz), 7.79 (1H, t, J=55.2 Hz), 12.12 (1H, s).

### Example 224

### 2-(Propylsulfanyl)-3-[4-(2,2,2-trifluoroethoxy)phenyl]-3,7-dihydro-4H-pyrrolo[2,3-d]pyrimidin-4-one

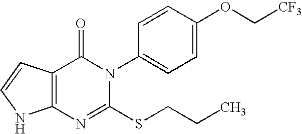

A 1 M aqueous solution of sodium hydrogen carbonate (0.59 ml) was added to a mixture of 2-thioxo-3-[4-(2,2,2-trifluoroethoxy)phenyl]-1,2,3,7-tetrahydro-4H-pyrrolo[2,3-d]pyrimidin-4-one (200 mg) obtained by the method of Example 26, or a method pursuant to thereto, 1-iodopropane (86 μl) and N,N-dimethylformamide (6 ml), and the resulting mixture was stirred for one hour at 100° C. The reaction mixture was returned to room temperature, and then the solvent was distilled off under reduced pressure. The resulting residue was diluted with ethyl acetate, and the dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (120 mg) was obtained as a white powder.

¹H NMR (300 MHz, DMSO-d₆) δ ppm 0.92 (3H, t, J=7.4 Hz), 1.54-1.70 (2H, m), 3.03 (2H, t, J=7.2 Hz), 4.87 (2H, q, J=9.0 Hz), 6.37-6.45 (1H, m), 6.93-7.00 (1H, m), 7.18 (2H, d, J=9.0 Hz), 7.29 (2H, d, J=9.0 Hz), 11.85 (1H, br. s.).

### Example 225

### 3-[4-(2,2,2-Trifluoroethoxy)phenyl]-2-[(2,2,2-trifluoroethyl)sulfanyl]-3,7-dihydro-4H-pyrrolo[2,3-d]pyrimidin-4-one

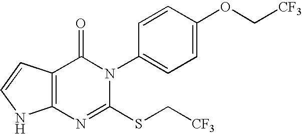

A 1 M aqueous solution of sodium hydrogen carbonate (0.59 ml) was added to a mixture of 2-thioxo-3-[4-(2,2,2-trifluoroethoxy)phenyl]-1,2,3,7-tetrahydro-4H-pyrrolo[2,3-d]pyrimidin-4-one (200 mg) obtained by the method of Example 26, or a method pursuant to thereto, 1,1,1-trifluoro-2-iodoethane (88 μl) and N,N-dimethylformamide (6 ml), and the resulting mixture was stirred for one hour at 100° C. The reaction mixture was returned to room temperature, and then the solvent was distilled off under reduced pressure. The resulting residue was diluted with ethyl acetate, and the dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (108 mg) was obtained as a white powder.

¹H NMR (300 MHz, DMSO-d₆) δ ppm 4.20 (2H, q, J=10.2 Hz), 4.89 (2H, q, J=9.0 Hz), 6.47 (1H, d, J=3.4 Hz), 7.04 (1H, d, J=3.4 Hz), 7.22 (2H, d, J=9.1 Hz), 7.37 (2H, d, J=9.1 Hz), 12.01 (1H, s).

### Example 226

### 2-(Cyclopropylmethoxy)-3-[4-(2,2,2-trifluoroethoxy)phenyl]-3,7-dihydro-4H-pyrrolo[2,3-d]pyrimidin-4-one

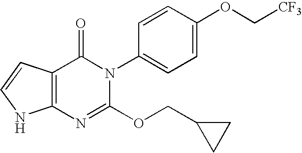

A tetrahydrofuran (10 ml) solution of cyclopropylmethanol (5 ml) was added dropwise to a mixture of sodium hydride (60% in oil, 60 mg) and tetrahydrofuran (10 ml). To the mixture, 2-(methylsulfonyl)-3-[4-(2,2,2-trifluoroethoxy)phenyl]-3,7-dihydro-4H-pyrrolo[2,3-d]pyrimidin-4-one (200 mg) obtained by the method of Example 220, or a method pursuant to thereto was added. The mixture was stirred overnight at room temperature, and then was concentrated under reduced pressure. Water was added to the residue, and the pH of the mixture was adjusted to about 6 with a 5% aqueous solution of citric acid. Then, the mixture was extracted with ethyl acetate. The organic layer was washed with water and saturated brine, dried over anhydrous sodium sulfate, and then concentrated under reduced pressure. The residue was purified by chromatography, and then was recrystallized from a mixed solvent of ethyl acetate/hexane. Thus, the title compound (74 mg) was obtained as a white solid.

¹H NMR (300 MHz, DMSO-d₆) δ ppm 0.16-0.23 (2H, m), 0.40-0.47 (2H, m), 1.03-1.18 (1H, m), 4.12 (2H, d, J=6.8 Hz), 4.84 (2H, q, J=8.9 Hz), 6.37 (1H, dd, J=3.3, 2.2 Hz), 6.88 (1H, dd, J=3.4, 2.3 Hz), 7.14 (2H, d, J=9.0 Hz), 7.24 (2H, d, J=9.0 Hz), 11.66 (1H, br. s.).

### Example 227

### 2-(2,2,2-Trifluoroethoxy)-3-[4-(2,2,2-trifluoroethoxy)phenyl]-3,7-dihydro-4H-pyrrolo[2,3-d]pyrimidin-4-one

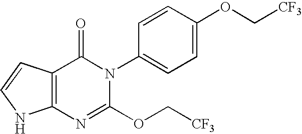

A tetrahydrofuran (10 ml) solution of 2,2,2-trifluoroethanol (5 ml) was added dropwise to a mixture of sodium hydride (60% in oil, 60 mg) and tetrahydrofuran (10 ml). To the mixture, 2-(methylsulfonyl)-3-[4-(2,2,2-trifluoroethoxy)phenyl]-3,7-dihydro-4H-pyrrolo[2,3-d]pyrimidin-4-one (200 mg) obtained by the method of Example 220, or a method pursuant to thereto was added. The mixture was stirred overnight at room temperature, and then was concentrated under reduced pressure. Water was added to the residue, and the pH of the mixture was adjusted to about 6 with a 5% aqueous solution of citric acid. Then, the mixture was extracted with ethyl acetate. The organic layer was washed with water and saturated brine, dried over anhydrous sodium sulfate, and then concentrated under reduced pressure. The residue was purified by chromatography, and then was recrystallized from a mixed solvent of ethyl acetate/hexane. Thus, the title compound (109 mg) was obtained as a pale brown solid.

¹H NMR (300 MHz, DMSO-d₆) δ ppm 4.84 (2H, q, J=9.0 Hz), 4.97 (2H, q, J=8.9 Hz), 6.43 (1H, dd, J=3.3, 2.0 Hz), 6.96 (1H, dd, J=3.2, 2.3 Hz), 7.15 (2H, d, J=9.0 Hz), 7.26 (2H, d, J=9.0 Hz), 11.84 (1H, br. s.).

### Example 228

### 2-(Propylamino)-3-[4-(2,2,2-trifluoroethoxy)phenyl]-3,7-dihydro-4H-pyrrolo[2,3-d]pyrimidin-4-one

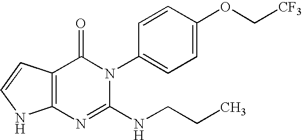

A mixture of 2-(methylsulfonyl)-3-[4-(2,2,2-trifluoroethoxy)phenyl]-3,7-dihydro-4H-pyrrolo[2,3-d]pyrimidin-4-one (200 mg) obtained by the method of Example 220, or a method pursuant to thereto, 1-propylamine (3 ml) and tetrahydrofuran (20 ml) was stirred for 3 days at room temperature, for one hour at 100° C. and for one hour at 120° C., and then was concentrated under reduced pressure. Water was added to the residue, and the mixture was extracted with ethyl acetate. The organic layer was washed with water and saturated brine, dried over anhydrous sodium sulfate, and then concentrated under reduced pressure. The residue was purified by chromatography, and then was recrystallized from a mixed solvent of ethyl acetate/hexane. Thus, the title compound (64 mg) was obtained as a white solid.

¹H NMR (300 MHz, DMSO-d₆) δ ppm 0.78 (3H, t, J=7.4 Hz), 1.40-1.54 (2H, m), 3.09-3.20 (2H, m), 4.84 (2H, q, J=8.7 Hz), 5.43 (1H, t, J=5.7 Hz), 6.22 (1H, dd, J=3.4, 1.9 Hz), 6.65 (1H, dd, J=3.2, 2.1 Hz), 7.20 (4H, s), 11.19 (1H, br. s.).

### Example 229

### 2-[(2-Hydroxyethyl)amino]-3-[4-(2,2,2-trifluoroethoxy)phenyl]-3,7-dihydro-4H-pyrrolo[2,3-d]pyrimidin-4-one

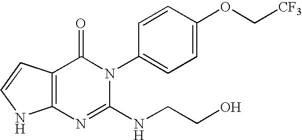

A mixture of 2-(methylsulfonyl)-3-[4-(2,2,2-trifluoroethoxy)phenyl]-3,7-dihydro-4H-pyrrolo[2,3-d]pyrimidin-4-one (200 mg) obtained by the method of Example 220, or a method pursuant to thereto, 2-aminoethanol (3 ml) and tetrahydrofuran (20 ml) was stirred for 3 days at 60° C., heated to reflux for 2 days, and then concentrated under reduced pressure. Water was added to the residue, and the mixture was extracted with ethyl acetate. The organic layer was washed with water and saturated brine, dried over anhydrous sodium sulfate, and then concentrated under reduced pressure. The residue was purified by chromatography, and then was recrystallized from a mixed solvent of ethyl acetate/hexane. Thus, the title compound (78 mg) was obtained as a white solid.

¹H NMR (300 MHz, DMSO-d₆) δ ppm 3.23-3.31 (2H, m), 3.40-3.49 (2H, m), 4.62 (1H, t, J=5.1 Hz), 4.84 (2H, q, J=9.0 Hz), 5.26 (1H, t, J=5.3 Hz), 6.23 (1H, dd, J=3.0, 1.9 Hz), 6.68 (1H, dd, J=3.0, 1.9 Hz), 7.17-7.26 (4H, m), 11.22(1H, br. s.).

### Example 230

### 2-(2-Hydroxyethoxy)-3-[4-(2,2,2-trifluoroethoxy)phenyl]-3,7-dihydro-4H-pyrrolo[2,3-d]pyrimidin-4-one

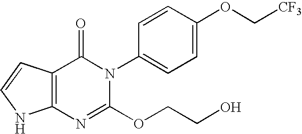

A tetrahydrofuran (20 ml) solution of ethane-1,2-diol (3 ml) was added dropwise to a mixture of sodium hydride (60% in oil, 100 mg) and tetrahydrofuran (10 ml). To the mixture, 2-(methylsulfonyl)-3-[4-(2,2,2-trifluoroethoxy)phenyl]-3,7-dihydro-4H-pyrrolo[2,3-d]pyrimidin-4-one (200 mg) obtained by the method of Example 220, or a method pursuant to thereto was added, and the resulting mixture was stirred for 2 hours at 60° C., and then was concentrated under reduced pressure. Water was added to the residue, and the pH of the mixture was adjusted to about 6 with a 5% aqueous solution of citric acid. Then, the mixture was extracted with ethyl acetate. The organic layer was washed with water and saturated brine, dried over anhydrous sodium sulfate, and then concentrated under reduced pressure. The residue was purified by chromatography, and then was recrystallized from a mixed solvent of ethyl acetate/hexane. Thus, the title compound (144 mg) was obtained as a pale brown solid.

¹H NMR (300 MHz, DMSO-d₆) δ ppm 3.55 (2H, q, J=5.0 Hz), 4.26-4.31 (2H, m), 4.70 (1H, t, J=5.3 Hz), 4.83 (2H, q, J=9.0 Hz), 6.37 (1H, dd, J=3.4, 2.3 Hz), 6.88 (1H, dd, J=3.4, 2.3 Hz), 7.12 (2H, d, J=9.1 Hz), 7.24 (2H, d, J=9.1 Hz), 11.68 (1H, br. s.).

### Example 231

### 6-Chloro-2-(ethylsulfanyl)-3-[4-(2,2,2-trifluoroethoxy)phenyl]-3,7-dihydro-4H-pyrrolo[2,3-d]pyrimidin-4-one

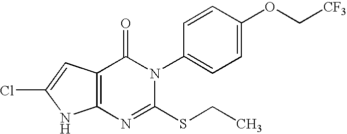

A mixture of 2-(ethylsulfanyl)-3-[4-(2,2,2-trifluoroethoxy)phenyl]-3,7-dihydro-4H-pyrrolo[2,3-d]pyrimidin-4-one (120 mg) obtained by the method of Example 194, or a method pursuant to thereto, N-chlorosuccinimide (47.7 mg) and N,N-dimethylformamide (2 ml) was heated to 50° C. The mixture was stirred for 1.5 hours. The reaction mixture was returned to room temperature, and then a 10% aqueous solution of sodium thiosulfate (3 ml) was added thereto. The mixture was stirred for 15 minutes at room temperature. Subsequently, water (5 ml) was added thereto, and the mixture was extracted with ethyl acetate. The extract was washed with water and saturated brine, dried over anhydrous magnesium sulfate, and then concentrated under reduced pressure. The resulting residue was purified by chromatography, and thus the title compound (70 mg) was obtained as a white solid, together with 5,6-dichloro-2-(ethylsulfanyl)-3-[4-(2,2,2-trifluoroethoxy)phenyl]-3,7-dihydro-4H-pyrrolo[2,3-d]pyrimidin-4-one (30 mg) of Example 234.

¹H NMR (400 MHz, DMSO-d₆) δ ppm 1.24 (3H, t, J=7.3 Hz), 3.02 (2H, q, J=7.3 Hz), 4.87 (2H, q, J=8.8 Hz), 6.44 (1 H., s), 7.18 (2H, d,. J=9.1 Hz), 7.31 (2H, d, J=9.0 Hz), 12.74 (1H, s).

### Example 232

### 6-Bromo-2-(ethylsulfanyl)-3-[4-(2,2,2-trifluoroethoxy)phenyl]-3,7-dihydro-4H-pyrrolo[2,3-d]pyrimidin-4-one

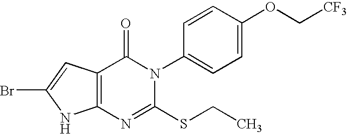

A mixture of 2-(ethylsulfanyl)-3-[4-(2,2,2-trifluoroethoxy)phenyl]-3,7-dihydro-4H-pyrrolo[2,3-d]pyrimidin-4-one (120 mg) obtained by the method of Example 194, or a method pursuant to thereto, N-bromosuccinimide (57.8 mg) and N,N-dimethylformamide (2 ml) was stirred for 10 minutes at room temperature. Subsequently, a 10% aqueous solution of sodium thiosulfate (3 ml) was added to the reaction mixture, and the resulting mixture was stirred for 15 minutes at room temperature. Water (5 ml) was added thereto, and the mixture was extracted with ethyl acetate. The extract was washed with water and saturated brine, dried over anhydrous magnesium sulfate, and then concentrated under reduced pressure. The resulting residue was purified by chromatography, and thus the title compound (45 mg) was obtained as a white solid, together with 5,6-dibromo-2-(ethylsulfanyl)-3-[4-(2,2,2-trifluoroethoxy)phenyl]-3,7-dihydro-4H-pyrrolo[2,3-d]pyrimidin-4-one (34 mg) of Example 233.

¹H NMR (400 MHz, DMSO-d₆) δ ppm 1.24 (3H, t, J=7.3 Hz), 3.02 (2H, q, J=7.3 Hz), 4.87 (2H, q, J=8.8 Hz), 6.53 (1H, s), 7.18 (2H, d, J=9.0 Hz), 7.30 (2H, d, J=9.0 Hz), 12.68 (1H, s).

### Example 233

### 5,6-Dibromo-2-(ethylsulfanyl)-3-[4-(2,2,2-trifluoroethoxy)phenyl]-3,7-dihydro-4H-pyrrolo[2,3-d]pyrimidin-4-one

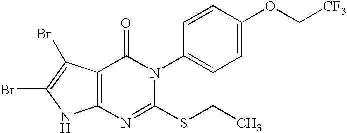

A mixture of 2-(ethylsulfanyl)-3-[4-(2,2,2-trifluoroethoxy)phenyl]-3,7-dihydro-4H-pyrrolo[2,3-d]pyrimidin-4-one (120 mg) obtained by the method of Example 194, or a method pursuant to thereto, N-bromosuccinimide (57.8 mg) and N,N-dimethylformamide (2 ml) was stirred for 10 minutes at room temperature. Subsequently, a 10% aqueous solution of sodium thiosulfate (3 ml) was added to the reaction mixture, and the resulting mixture was stirred for 15 minutes at room temperature. Water (5 ml) was added thereto, and the mixture was extracted with ethyl acetate. The extract was washed with water and saturated brine, dried over anhydrous magnesium sulfate, and then concentrated under reduced pressure. The resulting residue was purified by chromatography, and thus the title compound (34 mg) was obtained as a white solid, together with 6-bromo-2-(ethylsulfanyl)-3-[4-(2,2,2-trifluoroethoxy)phenyl]-3,7-dihydro-4H-pyrrolo[2,3-d]pyrimidin-4-one (45 mg) of Example 232.

¹H NMR (400 MHz, DMSO-d₆) δ ppm 1.24 (3H, t, J=7.3 Hz), 3.02 (2H, q, J=7.3 Hz), 4.87 (2H, q, J=8.8 Hz), 7.19 (2H, d, J=9.0 Hz), 7.33 (2H, d, J=9.0 Hz), 13.10 (1H, s).

### Example 234

### 5,6-Dichloro-2-(ethylsulfanyl)-3-[4-(2,2,2-trifluoroethoxy)phenyl]-3,7-dihydro-4H-pyrrolo[2,3-d]pyrimidin-4-one

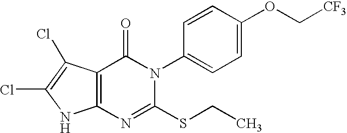

A mixture of 2-(ethylsulfanyl)-3-[4-(2,2,2-trifluoroethoxy)phenyl]-3,7-dihydro-4H-pyrrolo[2,3-d]pyrimidin-4-one (120 mg) obtained by the method of Example 194, or a method pursuant to thereto, N-chlorosuccinimide (47.7 mg) and N,N-dimethylformamide (2 ml) was heated to 50° C. The mixture was stirred for 1.5 hours. The reaction mixture was returned to room temperature, and then a 10% aqueous solution of sodium thiosulfate (3 ml) was added thereto. The mixture was stirred for 15 minutes at room temperature. Subsequently, water (5 ml) was added thereto, and the mixture was extracted with ethyl acetate. The extract was washed with water and saturated brine, dried over anhydrous magnesium sulfate, and then concentrated under reduced pressure. The resulting residue was purified by chromatography, and thus the title compound (30 mg) was obtained as a white solid, together with 6-chloro-2-(ethylsulfanyl)-3-[4-(2,2,2-trifluoroethoxy)phenyl]-3,7-dihydro-4H-pyrrolo[2,3-d]pyrimidin-4-one (70 mg) of Example 231.

¹H NMR (400 MHz, DMSO-d₆) δ ppm 1.24 (3H, t, J=7.3 Hz), 3.02 (2H, q, J=7.3 Hz), 4.87 (2H, q, J=8.8 Hz), 7.19 (2H, d, J=9.0 Hz), 7.33 (2H, d, J=9.0 Hz), 13.16 (1H, s).

### Example 235

### 2-(Methylsulfanyl)-3-[4-(trifluoromethoxy)phenyl]-3,7-dihydro-4H-pyrrolo[2,3-d]pyrimidin-4-one

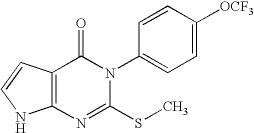

A 1 M aqueous solution of sodium hydrogen carbonate (0.61 ml) was added to a mixture of 2-thioxo-3-[4-(trifluoromethoxy)phenyl]-1,2,3,7-tetrahydro-4H-pyrrolo[2,3-d]pyrimidin-4-one (200 mg) obtained by the method of Example 37, or a method pursuant to thereto, iodomethane (80 μl) and N,N-dimethylformamide (6 ml), and the resulting mixture was stirred for 3 hours at 50° C. The reaction mixture was returned to room temperature, and then the solvent was distilled off under reduced pressure. The resulting residue was diluted with ethyl acetate, and the dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (159 mg) was obtained as a white powder.

¹H NMR (300 MHz, DMSO-d₆) δ ppm 2.45 (3H, s), 6.45 (1H, d, J=3.4 Hz), 7.00 (1H, d, J=3.4 Hz), 7.54 (4H, s), 11.94 (1H, br. s.).

### Example 236

### 3-[3-Chloro-4-(2,2,2-trifluoroethoxy)phenyl]-2-(methylsulfanyl)-3,7-dihydro-4H-pyrrolo[2,3-d]pyrimidin-4-one

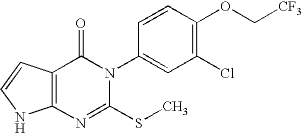

A 1 M aqueous solution of sodium hydrogen carbonate (0.53 ml) was added to a mixture of 3-[3-chloro-4-(2,2,2-trifluoroethoxy)phenyl]-2-thioxo-1,2,3,7-tetrahydro-4H-pyrrolo[2,3-d]pyrimidin-4-one (200 mg) obtained by the method of Example 38, or a method pursuant to thereto, iodomethane (80 μl) and N,N-dimethylformamide (5.3 ml), and the resulting mixture was stirred for 3 hours at 50° C. The reaction mixture was returned to room temperature, and then the solvent was distilled off under reduced pressure. The resulting residue was diluted with ethyl acetate, and the dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (168 mg) was obtained as a white powder.

¹H NMR (300 MHz, DMSO-d₆) δ ppm 2.44 (3H, s), 4.99 (2H, q, J=8.7 Hz), 6.44 (1H, d, J=3.4 Hz), 6.99 (1H, d, J=3.4 Hz), 7.33-7.45 (2H, m), 7.62 (1H, d, J=1.9 Hz), 11.91 (1H, s).

### Example 237

### 3-[3-Fluoro-4-(2,2,2-trifluoroethoxy)phenyl]-2-(methylsulfanyl)-3,7-dihydro-4H-pyrrolo[2,3-d]pyrimidin-4-one

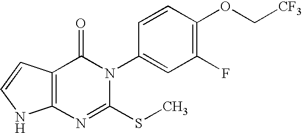

A 1 M aqueous solution of sodium hydrogen carbonate (0.56 ml) was added to a mixture of 3-[3-fluoro-4-(2,2,2-trifluoroethoxy)phenyl]-2-thioxo-1,2,3,7-tetrahydro-4H-pyrrolo[2,3-d]pyrimidin-4-one (200 mg) obtained by the method of Example 39, or a method pursuant to thereto, iodomethane (85 μl) and N,N-dimethylformamide (5.6 ml), and the resulting mixture was stirred for 2 hours at 50° C. The reaction mixture was returned to room temperature, and then the solvent was distilled off under reduced pressure. The resulting residue was diluted with ethyl acetate, and the dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (160 mg) was obtained as a white powder.

¹H NMR (300 MHz, DMSO-d₆) δ ppm 2.44 (3H, s), 4.97 (2H, q, J=8.7 Hz), 6.44 (1H, d, J=3.4 Hz), 6.99 (1H, d, J=3.4 Hz), 7.18-7.26 (1H, m), 7.36-7.45 (1H, m), 7.48(1H, dd, J=11.7, 2.3 Hz), 11.91 (1H, br. s.).

### Example 238

### 2-(Methylsulfanyl)-3-[3-methyl-4-(2,2,2-trifluoroethoxy)phenyl]-3,7-dihydro-4H-pyrrolo[2,3-d]pyrimidin-4-one

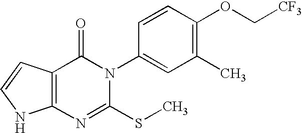

A 1 M aqueous solution of sodium hydrogen carbonate (0.56 ml) was added to a mixture of 3-[3-methyl-4-(2,2,2-trifluoroethoxy)phenyl]-2-thioxo-1,2,3,7-tetrahydro-4H-pyrrolo[2,3-d]pyrimidin-4-one (200 mg) obtained by the method of Example 40, or a method pursuant to thereto, iodomethane (56 μl) and N,N-dimethylformamide (6 ml), and the resulting mixture was stirred for 2 hours at 50° C. The reaction mixture was returned to room temperature, and then the solvent was distilled off under reduced pressure. The resulting residue was diluted with ethyl acetate, and the dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (150 mg) was obtained as a white powder.

¹H NMR (300 MHz, DMSO-d₆) δ ppm 2.21 (3H, s), 2.41 (3H, s), 4.86 (2H, q, J=8.8 Hz), 6.42 (1H, d, J=3.2 Hz), 6.98 (1H, d, J=3.2 Hz), 7.17 (3H, s), 11.87 (1H, br. s.).

### Example 239

### 2-(2,2,3,3,3-Pentafluoropropoxy)-3-[4-(2,2,2-trifluoroethoxy)phenyl]-3,7-dihydro-4H-pyrrolo[2,3-d]pyrimidin-4-one

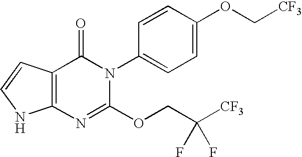

2-(Methylsulfinyl)-3-[4-(2,2,2-trifluoroethoxy)phenyl]-3,7-dihydro-4H-pyrrolo[2,3-d]pyrimidin-4-one (200 mg) obtained by the method of Example 260, or a method pursuant to thereto, and 2,2,3,3,3-pentafluoropropanol (0.161 ml) were dissolved in N,N-dimethylformamide (3 ml), and sodium hydride (60% in oil, 43.1 mg) was added to the solution with ice cooling. The mixture was stirred for 9 hours at room temperature. To the reaction mixture was added 1 M hydrochloric acid (1.5 ml), and the mixture was diluted with ethyl acetate (50 ml). The aqueous layer was removed therefrom, and then the organic layer was washed with water (10 ml) three times, and then was washed with saturated brine. Then, the resultant was dried over anhydrous magnesium sulfate, and then concentrated under reduced pressure. The residual beige solid was purified by chromatography to give the title compound (170 mg) as a white solid.

¹HNMR (300 MHz, DMSO-d₆) δ ppm 4.84 (2H, q, J=8.9 Hz), 5.03 (2H, t, J=13.1 Hz), 6.44 (1H, d, J=3.4 Hz), 6.96 (1H, d, J=3.4 Hz), 7.15 (2H, d, J=9.1 Hz), 7.24 (2H, d, J=9.1 Hz), 11.85 (1H, br. s.).

### Example 240

### 3-[3-Chloro-4-(2,2,2-trifluoroethoxy)phenyl]-3,7-dihydro-4H-pyrrolo[2,3-d]pyrimidin-4-one

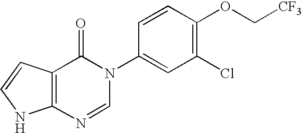

A mixture of ethyl 2-isothiocyanato-1H-pyrrol-3-carboxylate (500 mg) obtained by the method of Reference Example 36, or a method pursuant to thereto, 3-chloro-4-(2,2,2-trifluoroethoxy)aniline (575 mg) obtained by the method of Reference Example 23, or a method pursuant to thereto, and acetonitrile (20 ml) was heated to reflux for 2 hours. The mixture was ice-cooled, and then a 20% sodium ethoxide-ethanol solution (3.5 ml) and ethanol (3.5 ml) were added thereto. The mixture was stirred for 2 hours at 95° C. The reaction mixture was returned to room temperature, and the solvent was distilled off under reduced pressure. The residue was made basic with a 1 M aqueous solution of sodium hydroxide, and then was extracted with a mixed solvent of 25% tetrahydrofuran/diethyl ether. The organic layer obtained therefrom was washed with saturated brine, dried over anhydrous magnesium sulfate, and then concentrated under reduced pressure. The resulting residue was purified by chromatography, and thus the title compound (235 mg) was obtained as a white powder.

¹H NMR (300 MHz, DMSO-d₆) δ ppm 4.97 (2H, q, J=8.7 Hz), 6.52 (1H, d, J=3.3 Hz), 7.13 (1H, d, J=3.3 Hz), 7.42 (1H, d, J=8.7 Hz), 7.47 (1H, dd, J=8.7, 2.5 Hz), 7.70 (1H, d, J=2.4 Hz), 8.10 (1H, s), 12.05 (1H, s).

### Example 241

### 3-[4-(2,2,2-Trifluoroethoxy)phenyl]-3,7-dihydro-4H-pyrrolo[2,3-d]pyrimidin-4-one

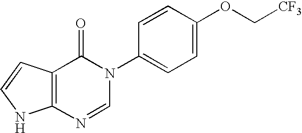

A mixture of 3-[3-chloro-4-(2,2,2-trifluoroethoxy)phenyl]-3,7-dihydro-4H-pyrrolo[2,3-d]pyrimidin-4-one (50 mg) obtained in Example 240, 10% palladium/activated carbon (50% hydrated, 50 mg), ammonium formate (88 mg) and methanol (5 ml) was heated to reflux for 4 hours. The reaction mixture was returned to room temperature, and was filtered. The resulting filtrate was concentrated under reduced pressure. The resulting residue was diluted with ethyl acetate, and the dilution was washed with water and saturated brine, dried over anhydrous magnesium sulfate, and then concentrated under reduced pressure. The resulting residue was purified by chromatography, and thus the title compound (21 mg) was obtained as a white powder.

¹H NMR (300 MHz, DMSO-d₆) δ ppm 4.85 (2H, q, J=9.0 Hz), 6.51 (1H, d, J=3.2 Hz), 7.12 (1H, d, J=3.2 Hz), 7.20 (2H, d, J=8.7 Hz), 7.42 (2H, d, J=8.7 Hz), 8.07 (1H, s), 12.02 (1H, br. s.).

### Example 242

### 2-(Methylsulfanyl)-3-[4-(3,3,3-trifluoropropyl)phenyl]-3,7-dihydro-4H-pyrrolo[2,3-d]pyrimidin-4-one

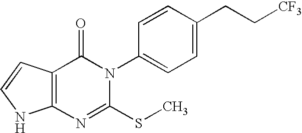

A mixture of 2-thioxo-3-[4-(3,3,3-trifluoropropyl)phenyl]-1,2,3,7-tetrahydro-4H-pyrrolo[2,3-d]pyrimidin-4-one (250 mg) obtained by the method of Example 42, or a method pursuant to thereto, iodomethane (0.5 ml), a 1 M aqueous solution of sodium hydrogen carbonate (0.85 ml) and N,N-dimethylformamide (20 ml) was stirred overnight at room temperature, and then was concentrated under reduced pressure. Ethyl acetate and water were added to the residue, and then the mixture was extracted with ethyl acetate. The organic layer was washed with water and saturated brine, dried over anhydrous sodium sulfate, and then concentrated under reduced pressure. The residue was purified by chromatography, and then was recrystallized from ethyl acetate. Thus, the title compound (130 mg) was obtained as a pale brown solid.

¹H NMR (300 MHz, DMSO-d₆) δ ppm 2.42 (3H, s), 2.58-2.77 (2H, m), 2.87-2.98 (2H, m), 6.43 (1H, d, J=3.4 Hz), 6.98 (1H, d, J=3.4 Hz), 7.26 (2H, d, J=8.4 Hz), 7.46 (2H, d, J=8.4 Hz), 11.89 (1H, s).

### Example 243

### 3-[4-(2-Cyclopropylethyl)phenyl]-2-(methylsulfanyl)-3,7-dihydro-4H-pyrrolo[2,3-d]pyrimidin-4-one

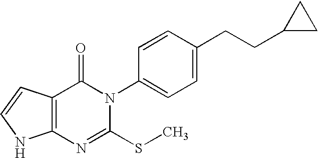

A mixture of 3-[4-(2-cyclopropylethyl)phenyl]-2-thioxo-1,2,3,7-tetrahydro-4H-pyrrolo[2,3-d]pyrimidin-4-one (300 mg) obtained by the method of Example 43, or a method pursuant to thereto, iodomethane (0.5 ml), a 1 M aqueous solution of sodium hydrogen carbonate (1.2 ml) and N,N-dimethylformamide (20 ml) was stirred overnight at room temperature, and then was concentrated under reduced pressure. Ethyl acetate and water were added to the residue, and then the mixture was extracted with ethyl acetate. The organic layer was washed with water and saturated brine, dried over anhydrous sodium sulfate, and then concentrated under reduced pressure. The residue was purified by chromatography and reverse phase chromatography, and then was recrystallized from a mixed solvent of ethyl acetate/hexane. Thus, the title compound (26 mg) was obtained as a pale brown solid.

¹H NMR (300 MHz, DMSO-d₆) δ ppm 0.04-0.13 (2H, m), 0.38-0.46 (2H, m), 0.66-0.81 (1H, m), 1.48-1.59 (2H, m), 2.42 (3H, s), 2.71-2.80(2H, m), 6.42(1H, d, J=3.3 Hz), 6.98 (1H, d, J=3.3 Hz), 7.20 (2H, d, J=8.2 Hz), 7.35 (2H, d, J=8.2 Hz), 11.88 (1H, br. s.).

### Example 244

### 2-(2,2-Difluoroethoxy)-3-[4-(2,2,2-trifluoroethoxy)phenyl]-3,7-dihydro-4H-pyrrolo[2,3-d]pyrimidin-4-one

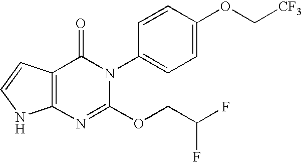

A tetrahydrofuran (10 ml) solution of 2,2-difluoroethanol (0.4 ml) was added dropwise to a mixture of sodium hydride (60% in oil, 60 mg) and tetrahydrofuran (10 ml). To the mixture, 2-(methylsulfonyl)-3-[4-(2,2,2-trifluoroethoxy)phenyl]-3,7-dihydro-4H-pyrrolo[2,3-d]pyrimidin-4-one (250 mg) obtained by the method of Example 220, or a method pursuant to thereto was added, and the resulting mixture was stirred for one hour at 60° C., and then was concentrated under reduced pressure. Water was added to the residue, and the pH of the mixture was adjusted to about 6 with a 5% aqueous solution of citric acid. Then, the mixture was extracted with ethyl acetate. The organic layer was washed with water and saturated brine, dried over anhydrous sodium sulfate, and then concentrated under reduced pressure. The residue was purified by chromatography, and then was recrystallized from a mixed solvent of ethyl acetate/hexane. Thus, the title compound (143 mg) was obtained as a white solid.

¹H NMR (300 MHz, DMSO-d₆) δ ppm 4.56 (2H, td, J=14.8, 3.4 Hz), 4.84 (2H, q, J=9.1 Hz), 6.24 (1H, tt, J=54.3, 3.4 Hz), 6.41 (1H, d, J=3.4 Hz), 6.93 (1H, d, J=3.4 Hz), 7.14 (2H, d, J=9.1 Hz), 7.25 (2H, d, J=9.1 Hz), 11.79 (1H, s).

### Example 245

### 2-Amino-3-[4-(2,2,2-trifluoroethoxy)phenyl]-3,7-dihydro-4H-pyrrolo[2,3-d]pyrimidin-4-one

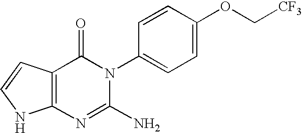

A mixture of 2-(methylsulfonyl)-3-[4-(2,2,2-trifluoroethoxy)phenyl]-3,7-dihydro-4H-pyrrolo[2,3-d]pyrimidin-4-one (300 mg) obtained by the method of Example 220, or a method pursuant to thereto, hydrazine monohydrate (0.5 ml) and ethanol (5 ml) was stirred overnight at 50° C. Water was added thereto, and the mixture was extracted with ethyl acetate. The organic layer was washed with water and saturated brine, dried over anhydrous sodium sulfate, and then concentrated under reduced pressure. To the residue, formic acid (5 ml) was added, and the resulting mixture was stirred overnight at 90° C., and then was concentrated under reduced pressure. Water and a 5% aqueous solution of sodium hydrogen carbonate were added to the residue, and the mixture was extracted with ethyl acetate. The organic layer was washed with water and saturated brine, dried over anhydrous sodium sulfate, and then concentrated under reduced pressure. The residue was purified by chromatography, and then was recrystallized from ethyl acetate. Thus, the title compound (45 mg) was obtained as a brown solid.

¹H NMR (300 MHz, DMSO-d₆) δ ppm 4.83 (2H, q, J=9.1 Hz), 5.89 (2H, br. s.), 6.22 (1H, dd, J=3.2, 2.1 Hz), 6.66 (1H, dd, J=3.4, 2.3 Hz), 7.15-7.26 (4H, m), 11.03 (1H, br. s.).

### Example 246

### 2-(4-Fluorophenoxy)-3-[4-(2,2,2-trifluoroethoxy)phenyl]-3,7-dihydro-4H-pyrrolo[2,3-d]pyrimidin-4-one

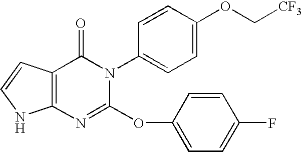

A tetrahydrofuran (10 ml) solution of 4-fluorophenol (112 mg) was added dropwise to a mixture of sodium hydride (60% in oil, 32 mg) and tetrahydrofuran (10 ml) in an ice water bath. To the mixture, 2-(methylsulfonyl)-3-[4-(2,2,2-trifluoroethoxy)phenyl]-3,7-dihydro-4H-pyrrolo[2,3-d]pyrimidin-4-one (250 mg) obtained by the method of Example 220, or a method pursuant to thereto was added, and the resulting mixture was stirred for 2 hours at 60° C., and then was concentrated under reduced pressure. Water was added to the residue, and the pH of the mixture was adjusted to about 6 with a 5% aqueous solution of citric acid. Then, the mixture was extracted with ethyl acetate. The organic layer was washed with water and saturated brine, dried over anhydrous sodium sulfate, and then concentrated under reduced pressure. The residue was purified by chromatography, and then was recrystallized from a mixed solvent of ethyl acetate/hexane. Thus, the title compound (127 mg) was obtained as a pale red solid.

¹H NMR (300 MHz, DMSO-d₆) δ ppm 4.83 (2H, q, J=8.9 Hz), 6.42 (1H, d, J=3.4 Hz), 6.89 (1H, d, J=3.4 Hz), 7.18 (2H, d, J=9.0 Hz), 7.21-7.34 (4H, m), 7.47 (2H, d, J=9.0 Hz), 11.80 (1H, s).

### Example 247

### 2-(Cyclobutyloxy)-3-[4-(2,2,2-trifluoroethoxy)phenyl]-3,7-dihydro-4H-pyrrolo[2,3-d]pyrimidin-4-one

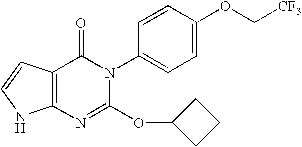

Cyclobutanol (1 g) was added dropwise to a mixture of sodium hydride (60% in oil, 32 mg) and tetrahydrofuran (20 ml) in an ice water bath. To the mixture, 2-(methylsulfonyl)-3-[4-(2,2,2-trifluoroethoxy)phenyl]-3,7-dihydro-4H-pyrrolo[2,3-d]pyrimidin-4-one (250 mg) obtained by the method of Example 220, or a method pursuant to thereto was added. The mixture was stirred for 2 hours at 70° C., and then was concentrated under reduced pressure. Water was added to the residue, and the pH of the mixture was adjusted to about 6 with a 5% aqueous solution of citric acid. Then, the mixture was extracted with ethyl acetate. The organic layer was washed with water and saturated brine, dried over anhydrous sodium sulfate, and then concentrated under reduced pressure. The residue was purified by chromatography, and then was recrystallized from a mixed solvent of ethyl acetate/hexane. Thus, the title compound (84 mg) was obtained as a white solid.

¹H NMR (300 MHz, DMSO-d₆) δ ppm 1.50-1.77 (2H, m), 1.79-1.96 (2H, m), 2.24-2.38 (2H, m), 4.84 (2H, q, J=8.7 Hz), 5.07 (1H, quin, J=7.4 Hz), 6.36 (1H, d, J=3.4 Hz), 6.87 (1H, d, J=3.4 Hz), 7.14 (2H, d, J=9.1 Hz), 7.24 (2H, d, J=9.1 Hz), 11.64 (1H, s).

### Example 248

### 3-[4-(2,2-Difluoroethoxy)phenyl]-2-(ethylsulfanyl)-3,7-dihydro-4H-pyrrolo[2,3-d]pyrimidin-4-one

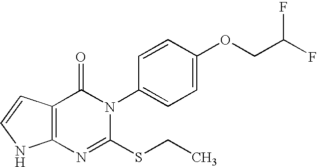

A mixture of 3-[4-(2,2-difluoroethoxy)phenyl]-2-thioxo-1,2,3,7-tetrahydro-4H-pyrrolo[2,3-d]pyrimidin-4-one (291 mg) obtained in Example 44, iodoethane (0.64 ml), a 1 M aqueous solution of sodium hydrogen carbonate (1.0 ml) and N,N-dimethylformamide (20 ml) was stirred overnight at room temperature, and then was concentrated under reduced pressure. Water was added to the residue, and then the mixture was extracted with ethyl acetate. The organic layer was washed with water and saturated brine, dried over anhydrous sodium sulfate, and then concentrated under reduced pressure. The residue was purified by chromatography, and then was recrystallized from a mixed solvent of ethyl acetate/hexane. Thus, the title compound (210 mg) was obtained as a white solid.

¹H NMR (300 MHz, DMSO-d₆) δ ppm 1.25 (3H, t, J=7.4 Hz), 3.03 (2H, q, J=7.4 Hz), 4.40 (2H, td, J=14.8, 3.4 Hz), 6.44 (1H, tt, J=54.5, 3.4 Hz), 6.42 (1H, d, J=3.4 Hz), 6.97 (1H, d, J=3.4 Hz), 7.13 (2H, d, J=8.9 Hz), 7.25 (2H, d, J=8.9 Hz), 11.85 (1H, s).

### Example 249

### 2-(Ethylsulfanyl)-3-[4-(2,2,3,3,3-pentafluoropropoxy)phenyl]-3,7-dihydro-4H-pyrrolo[2,3-d]pyrimidin-4-one

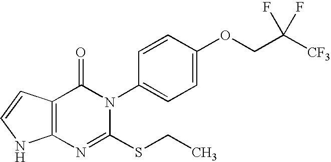

A mixture of 3-[4-(2,2,3,3,3-pentafluoropropoxy)phenyl]-2-thioxo-1,2,3,7-tetrahydro-4H-pyrrolo[2,3-d]pyrimidin-4-one (313 mg) obtained in Example 45, iodoethane (0.64 ml), a 1 M aqueous solution of sodium hydrogen carbonate (1.0 ml) and N,N-dimethylformamide (20 ml) was stirred overnight at room temperature, and then was concentrated under reduced pressure. Water was added to the residue, and then the mixture was extracted with ethyl acetate. The organic layer was washed with water and saturated brine, dried over anhydrous sodium sulfate, and then concentrated under reduced pressure. The residue was purified by chromatography, and then was recrystallized from a mixed solvent of ethyl acetate/hexane. Thus, the title compound (175 mg) was obtained as a white solid.

¹H NMR (300 MHz, DMSO-d₆) δ ppm 1.25 (3H, t, J=7.3 Hz), 3.04 (2H, q, J=7.3 Hz), 4.94 (2H, t, J=13.1 Hz), 6.43 (1H, d, J=3.4 Hz), 6.97 (1H, d, J=3.4 Hz), 7.19 (2H, d, J=9.1 Hz), 7.29 (2H, d, J=9.1 Hz), 11.86 (1H, s).

### Example 250

### 2-Ethoxy-4-oxo-3-[4-(2,2,2-trifluoroethoxy)phenyl]-4,7-dihydro-3H-pyrrolo[2,3-d]pyrimidin-6-yl acetate

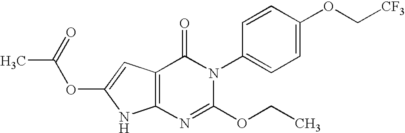

A mixture of 2-ethoxy-3-[4-(2,2,2-trifluoroethoxy)phenyl]-3,7-dihydro-4H-pyrrolo[2,3-d]pyrimidin-4-one (490 mg) obtained by the method of Example 209, or a method pursuant to thereto, iodosobenzene diacetate (600 mg) and acetic acid (20 ml) was stirred for 2 hours at 100° C., and then was concentrated under reduced pressure. Toluene was added to the residue, and the mixture was concentrated again under reduced pressure. The residue was purified by chromatography, and thus the title compound (206 mg) was obtained as a pale brown powder. Furthermore, 2-ethoxy-3-[4-(2,2,2-trifluoroethoxy)phenyl]-5,7-dihydro-3H-pyrrolo[2,3-d]pyrimidine-4,6-dione (105 mg) of Example 266 was also obtained.

¹H NMR (300 MHz, DMSO-d₆) δ ppm 1.15 (3H, t, J=7.2 Hz), 2.07 (3H, s), 4.37 (2H, q, J=7.2 Hz), 4.83 (2H, q, J=8.7 Hz), 5.95 (1H, s), 7.13 (2H, d, J=9.1 Hz), 7.19-7.33 (2H, m), 11.33 (1H, s).

### Example 251

### 2-[(1-Methylethyl)sulfanyl]-3-[4-(2,2,2-trifluoroethoxy)phenyl]-3,7-dihydro-4H-pyrrolo[2,3-d]pyrimidin-4-one

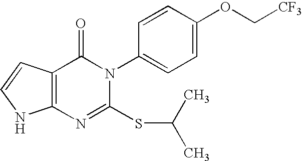

A 1 M aqueous solution of sodium hydrogen carbonate (0.58 ml) was added to a mixture of 2-thioxo-3-[4-(2,2,2-trifluoroethoxy)phenyl]-1,2,3,7-tetrahydro-4H-pyrrolo[2,3-d]pyrimidin-4-one (200 mg) obtained by the method of Example 26, or a method pursuant to thereto, 2-iodopropane (88 μl) and N,N-dimethylformamide (5 ml), and the resulting mixture was stirred for 3 hours at 100° C. The reaction mixture was returned to room temperature, and then the solvent was distilled off under reduced pressure. The resulting residue was diluted with ethyl acetate, and the dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (180 mg) was obtained as a white powder.

¹H NMR (300 MHz, DMSO-d₆) δ ppm 1.30 (6 H, d, J=6.8 Hz), 3.72-3.92 (1H, m), 4.86 (2H, q, J=9.1 Hz), 6.42 (1H, d, J=3.4 Hz), 6.97 (1H, d, J=3.4 Hz), 7.17 (2H, d, J=9.0 Hz), 7.27 (2H, d, J=9.0 Hz), 11.86 (1H, s).

### Example 252

### 2-(Butylsulfanyl)-3-[4-(2,2,2-trifluoroethoxy)phenyl]-3,7-dihydro-4H-pyrrolo[2,3-d]pyrimidin-4-one

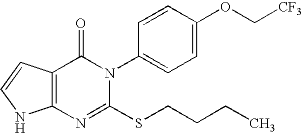

A 1 M aqueous solution of sodium hydrogen carbonate (0.58 ml) was added to a mixture of 2-thioxo-3-[4-(2,2,2-trifluoroethoxy)phenyl]-1,2,3,7-tetrahydro-4H-pyrrolo[2,3-d]pyrimidin-4-one (200 mg) obtained by the method of Example 26, or a method pursuant to thereto, 1-iodobutane (100 μl) and N,N-dimethylformamide (5 ml), and the resulting mixture was stirred for 3 hours at 100° C. The reaction mixture was returned to room temperature, and then the solvent was distilled off under reduced pressure. The resulting residue was diluted with ethyl acetate, and the dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (190 mg) was obtained as a white powder.

¹H NMR (300 MHz, DMSO-d₆) δ ppm 0.87 (3H, t, J=7.2 Hz), 1.27-1.42 (2H, m), 1.50-1.66 (2H, m), 3.05 (2H, t, J=7.2 Hz), 4.86 (2H, q, J=9.1 Hz), 6.42 (1H, d, J=3.4 Hz), 6.97 (1H, d, J=3.4 Hz), 7.18 (2H, d, J=8.7 Hz), 7.29 (2H, d, J=8.7 Hz), 11.84 (1H, br. s.).

### Example 253

### 3-[4-(2,2,2-Trifluoroethoxy)phenyl]-2-[(trifluoromethyl)sulfanyl]-3,7-dihydro-4H-pyrrolo[2,3-d]pyrimidin-4-one

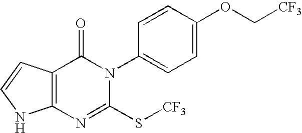

To a tetrahydrofuran (5 ml) suspension of 2-thioxo-3-[4-(2,2,2-trifluoroethoxy)phenyl]-1,2,3,7-tetrahydro-4H-pyrrolo[2,3-d]pyrimidin-4-one (200 mg) obtained by the method of Example 26, or a method pursuant to thereto, sodium hydride (60% in oil, 25.6 mg) was added under ice cooling, and the resulting mixture was stirred for 30 minutes under ice cooling. Then, 5-(trifluoromethyl)dibenzo[b,d]thiophenium trifluoromethanesulfonate (257 mg) was added thereto. The reaction mixture was stirred for 2 hours at room temperature, and then was diluted with ethyl acetate. This dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (10.3 mg) was obtained as a white powder.

¹H NMR (300 MHz, DMSO-d₆) δ ppm 4.89 (2H, q, J=8.9 Hz), 6.50-6.56 (1H, m), 7.09-7.16 (1H, m), 7.23 (2H, d, J=8.9 Hz), 7.48 (2H, d, J=8.9 Hz), 12.26 (1H, br. s.).

### Example 254

### 2-(Ethylsulfanyl)-3-[4-(trifluoromethoxy)phenyl]-3,7-dihydro-4H-pyrrolo[2,3-d]pyrimidin-4-one

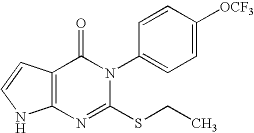

A 1 M aqueous solution of sodium hydrogen carbonate (0.31 ml) was added to a mixture of 2-thioxo-3-[4-(trifluoromethoxy)phenyl]-1,2,3,7-tetrahydro-4H-pyrrolo[2,3-d]pyrimidin-4-one (100 mg) obtained by the method of Example 37, or a method pursuant to thereto, iodoethane (97 mg) and N,N-dimethylformamide (5 ml), and the resulting mixture was stirred for 2 hours at 70° C. The reaction mixture was returned to room temperature, and then the solvent was distilled off under reduced pressure. The resulting residue was diluted with ethyl acetate, and the dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (78 mg) was obtained as a white powder.

¹H NMR (300 MHz, DMSO-d₆) δ ppm 1.26 (3H, t, J=7.2 Hz), 3.07 (2H, q, J=7.2 Hz), 6.45 (1H, d, J=3.4 Hz), 7.00 (1H, d, J=3.4 Hz), 7.48-7.57 (4H, m), 11.92 (1H, br. s.).

### Example 255

### 2-[(Cyclopropylmethyl)sulfanyl]-3-[4-(2,2,2-trifluoroethoxy)phenyl]-3,7-dihydro-4H-pyrrolo[2,3-d]pyrimidin-4-one

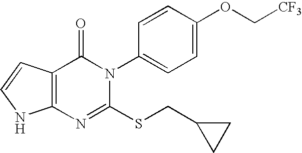

A 1 M aqueous solution of sodium hydrogen carbonate (0.58 ml) was added to a mixture of 2-thioxo-3-[4-(2,2,2-trifluoroethoxy)phenyl]-1,2,3,7-tetrahydro-4H-pyrrolo[2,3-d]pyrimidin-4-one (200 mg) obtained by the method of Example 26, or a method pursuant to thereto, (bromomethyl)cyclopropane (46 μl) and N,N-dimethylformamide (5 ml), and the resulting mixture was stirred for 2 hours at 90° C. The reaction mixture was returned to room temperature, and then the solvent was distilled off under reduced pressure. The resulting residue was diluted with ethyl acetate, and the dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (200 mg) was obtained as a white powder.

¹H NMR (300 MHz, DMSO-d₆) δ ppm 0.21-0.28 (2H, m), 0.47-0.55 (2H, m), 1.03-1.16 (1H, m), 3.00 (2H, d, J=7.2 Hz), 4.87 (2H, q, J=9.0 Hz), 6.42 (1H, d, J=3.4 Hz), 6.97 (1H, d, J=3.4 Hz), 7.19 (2H, d, J=8.7 Hz), 7.30 (2H, d, J=8.7 Hz), 11.87 (1H, br. s.).

### Example 256

### 2-(Cyclopropylsulfanyl)-3-[4-(2,2,2-trifluoroethoxy)phenyl]-3,7-dihydro-4H-pyrrolo[2,3-d]pyrimidin-4-one

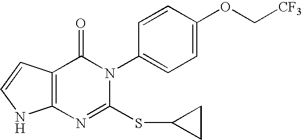

### Example 256a

To an acetonitrile (10 ml) solution of 2-thioxo-3-[4-(2,2,2-trifluoroethoxy)phenyl]-1,2,3,7-tetrahydro-4H-pyrrolo[2,3-d]pyrimidin-4-one (300 mg) obtained by the method of Example 26, or a method pursuant to thereto, an aqueous solution (10 ml) of sodium periodate (226 mg) was added at room temperature, and the resulting mixture was stirred for 30 minutes. The solvent was distilled off under reduced pressure, and then the resulting residue was diluted with ethyl acetate. The dilution was washed with water and saturated brine, dried over anhydrous magnesium sulfate, and then concentrated under reduced pressure. The resulting residue was purified by chromatography, and thus 2-({4-oxo-3-[4-(2,2,2-trifluoroethoxy)phenyl]-4,4a,7,7a-tetrahydro-3H-pyrrolo[2,3-d]pyrimidin-2-yl}disulfanyl)-3-[4-(2,2,2-trifluoroethoxy)phenyl]-3,7-dihydro-4H-pyrrolo[2,3-d]pyrimidin-4-one (280 mg) was obtained as a whitish yellow powder.

¹H NMR (300 MHz, DMSO-d₆) δ ppm 4.90 (4H, q, J=8.8 Hz), 6.43-6.49 (2H, m), 7.00-7.08 (2H, m), 7.26 (4H, d, J=8.7 Hz), 7.52 (4H, d, J=9.1 Hz), 11.95 (2H, br. s.).

### Example 256b

To a tetrahydrofuran (2 ml) solution of cyclopropyl magnesium bromide (0.5 M tetrahydrofuran solution, 5.88 ml), a tetrahydrofuran (10 ml) solution of 2-({4-oxo-3-[4-(2,2,2-trifluoroethoxy)phenyl]-4,4a,7,7a-tetrahydro-3H-pyrrolo[2,3-d]pyrimidin-2-yl}disulfanyl)-3-[4-(2,2,2-trifluoroethoxy)phenyl]-3,7-dihydro-4H-pyrrolo[2,3-d]pyrimidin-4-one (200 mg) obtained in Example (256a) was added dropwise at room temperature, and the resulting mixture was stirred for 3 hours. A saturated aqueous solution of ammonium chloride was added to the reaction mixture and the mixture was extracted with ethyl acetate. The organic layer was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, 2-(cyclopropylsulfanyl)-3-[4-(2,2,2-trifluoroethoxy)phenyl]-3,7-dihydro-4H-pyrrolo[2,3-d]pyrimidin-4-one (56 mg) was obtained as a white powder.

¹H NMR (300 MHz, DMSO-d₆) δ ppm 0.52-0.60 (2H, m), 0.98-1.06 (2H, m), 2.23-2.33 (1H, m), 4.85 (2H, q, J=8.7 Hz), 6.43 (1H, d, J=3.4 Hz), 6.98 (1H, d, J=3.4 Hz), 7.17 (2H, d, J=9.0 Hz), 7.27 (2H, d, J=9.0 Hz), 11.93 (1H, s).

### Example 257

### 2-(Methylsulfanyl)-4-oxo-3-[4-(2,2,2-trifluoroethoxy)phenyl]-4,7-dihydro-3H-pyrrolo[2,3-d]pyrimidin-6-yl acetate

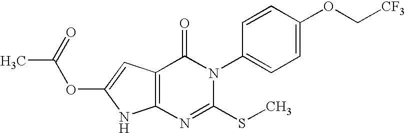

A mixture of 2-(methylsulfanyl)-3-[4-(2,2,2-trifluoroethoxy)phenyl]-3,7-dihydro-4H-pyrrolo[2,3-d]pyrimidin-4-one (1.00 g) obtained by the method of Example 212, or a method pursuant to thereto, iodosobenzene diacetate (1.00 g) and acetic acid (50 ml) was stirred for 2 hours at 100° C. and was concentrated under reduced pressure. Toluene was added to the residue, and the mixture was concentrated again under reduced pressure. The residue was purified by chromatography to give the title compound (263 mg) as a pale brown powder. Furthermore, 2-(methylsulfanyl)-3-[4-(2,2,2-trifluoroethoxy)phenyl]-5,7-dihydro-3H-pyrrolo[2,3-d]pyrimidine-4,6-dione (425 mg) of Example 267 was also obtained.

¹H NMR (300 MHz, DMSO-d₆) δ ppm 2.08 (3H, s), 2.44 (3H, s), 4.86 (2H, q, J=8.9 Hz), 5.96 (1H, s), 7.20 (2H, d, J=9.2 Hz), 7.27-7.40 (2H, m), 11.38 (1H, s).

### Example 258

### 2-Ethoxy-7-methyl-3-[4-(2,2,2-trifluoroethoxy)phenyl]-3,7-dihydro-4H-pyrrolo[2,3-d]pyrimidin-4-one

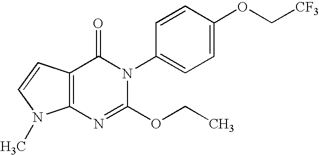

A mixture of 2-ethoxy-3-[4-(2,2,2-trifluoroethoxy)phenyl]-3,7-dihydro-4H-pyrrolo[2,3-d]pyrimidin-4-one (600 mg) obtained by the method of Example 209, or a method pursuant to thereto, sodium hydride (60% in oil, 80 mg) and N,N-dimethylformamide (20 ml) in an ice water bath, was stirred for 10 minutes. Iodomethane (0.6 ml) was added thereto, and the resulting mixture was stirred overnight at room temperature. Subsequently, water was added to this mixture, and the pH of the mixture was adjusted to about 6 with a 5% aqueous solution of citric acid. Then, the mixture was extracted with ethyl acetate. The organic layer was washed with water and saturated brine, dried over anhydrous sodium sulfate, and then concentrated under reduced pressure. The residue was purified by chromatography, and then was recrystallized from a mixed solvent of ethyl acetate/hexane. Thus, the title compound (613 mg) was obtained as a pale brown solid.

¹H NMR (300 MHz, DMSO-d₆) δ ppm 1.17 (3H, t, J=6.9 Hz), 3.66 (3H, s), 4.36 (2H, q, J=6.9 Hz), 4.83 (2H, q, J=9.0 Hz), 6.39 (1H, d, J=3.4 Hz), 6.95 (1H, d, J=3.4 Hz), 7.13 (2H, d, J=8.9 Hz), 7.22 (2H, d, J=8.9 Hz).

### Example 259

### 2-Ethoxy-7-methyl-4-oxo-3-[4-(2,2,2-trifluoroethoxy)phenyl]-4,7-dihydro-3H-pyrrolo[2,3-d]pyrimidin-6-yl acetate

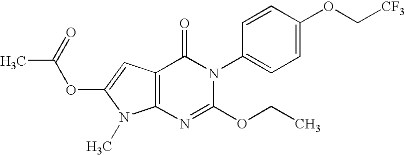

A mixture of 2-ethoxy-7-methyl-3-[4-(2,2,2-trifluoroethoxy)phenyl]-3,7-dihydro-4H-pyrrolo[2,3-d]pyrimidin-4-one (460 mg) obtained in Example 258, iodosobenzene diacetate (403 mg) and acetic acid (30 ml) was stirred for 2 hours at 100° C., and then was concentrated under reduced pressure. Toluene was added to the residue, and the mixture was concentrated again under reduced pressure. The residue was purified by chromatography, and thus the title compound (75 mg) was obtained as a pale brown powder. Furthermore, 2-ethoxy-7-methyl-3-[4-(2,2,2-trifluoroethoxy)phenyl]-5,7-dihydro-3H-pyrrolo[2,3-d]pyrimidine-4,6-dione (106 mg) of Example 268 was also obtained.

¹H NMR (300 MHz, DMSO-d₆) δ ppm 1.19 (3H, t, J=7.0 Hz), 2.08 (3H, s), 3.11 (3H, s), 4.46 (2H, q, J=7.0 Hz), 4.83 (2H, q, J=8.7 Hz), 6.01 (1H, s), 7.14 (2H, d, J=9.1 Hz), 7.20-7.32 (2H, m).

### Example 260

### 2-(Methylsulfinyl)-3-[4-(2,2,2-trifluoroethoxy)phenyl]-3,7-dihydro-4H-pyrrolo[2,3-d]pyrimidin-4-one

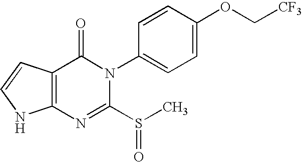

To a methanol (400 ml) solution of 2-(methylsulfanyl)-3-[4-(2,2,2-trifluoroethoxy)phenyl]-3,7-dihydro-4H-pyrrolo[2,3-d]pyrimidin-4-one (10.81 g) obtained by the method of Example 212, or a method pursuant to thereto, an aqueous solution (100 ml) of Oxone (registered trademark) monopersulfate compound (19.79 g) was added at room temperature, and then the mixture was stirred for 30 minutes at 70° C. The reaction mixture was returned to room temperature, and then methanol was distilled off under reduced pressure. Precipitates generated therefrom were collected by filtration, washed with water, and dried under reduced pressure. Thus, the title compound (10.95 g) was obtained as a pale brown powder.

¹H NMR (300 MHz, DMSO-d₆) δ ppm 2.72 (3H, s), 4.87 (2H, q, J=8.9 Hz), 6.59 (1H, dd, J=3.4, 2.1 Hz), 7.10-7.39 (3H, m), 7.39-7.54 (2H, m), 12.42 (1H, br. s.).

### Example 261

### 2-(3-Ethoxypropoxy)-3-[4-(2,2,2-trifluoroethoxy)phenyl]-3,7-dihydro-4H-pyrrolo[2,3-d]pyrimidin-4-one

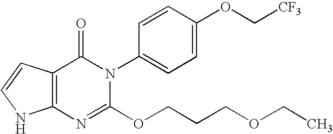

Sodium hydride (60% in oil, 240 mg) was added to a solution of 3-ethoxypropan-1-ol (625 mg) in N,N-dimethylformamide (10 ml) at room temperature, and the resulting mixture was stirred for 30 minutes. Then, a solution N,N-dimethylformamide (20 ml) of 2-(methylsulfinyl)-3-[4-(2,2,2-trifluoroethoxy)phenyl]-3,7-dihydro-4H-pyrrolo[2,3-d]pyrimidin-4-one (794 mg) obtained by the method of Example 260, or a method pursuant to thereto was added thereto dropwise at room temperature. The reaction mixture was stirred for 2 hours at room temperature, and then the solvent was distilled off under reduced pressure. The resulting residue was diluted with ethyl acetate, and the dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (559 mg) was obtained as a white powder.

¹H NMR (300 MHz, DMSO-d₆) δ ppm 1.03 (3H, t, J=7.1 Hz), 1.69-1.79 (2H, m), 3.22 (2H, t, J=6.3 Hz), 3.25-3.34 (2H, m), 4.29 (2H, t, J=6.2 Hz), 4.83 (2H, q, J=8.9 Hz), 6.38 (1H, d, J=3.4 Hz), 6.88 (1H, d, J=3.4 Hz), 7.15 (2H, d, J=9.0 Hz), 7.23 (2H, d, J=9.0 Hz), 11.69 (1H, br. s.).

### Example 262

### 2-(Ethylsulfanyl)-3-[3-fluoro-4-(2,2,2-trifluoroethoxy)phenyl]-3,7-dihydro-4H-pyrrolo[2,3-d]pyrimidin-4-one

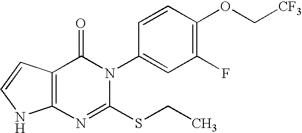

A 1 M aqueous solution of sodium hydrogen carbonate (20.4 ml) was added to a mixture of 3-[3-fluoro-4-(2,2,2-trifluoroethoxy)phenyl]-2-thioxo-1,2,3,7-tetrahydro-4H-pyrrolo[2,3-d]pyrimidin-4-one (7.66 g) obtained by the method of Example 39, or a method pursuant to thereto, iodoethane (2.45 ml) and N,N-dimethylformamide (50 ml), and the resulting mixture was stirred for one hour at 50° C. The reaction mixture was returned to room temperature, and then was diluted with ethyl acetate. The dilution was washed with water and saturated brine, dried over anhydrous magnesium sulfate, and then concentrated under reduced pressure. The resulting residue was purified by chromatography, and thus the title compound (8.10 g) was obtained as a white powder.

¹H NMR (300 MHz, DMSO-d₆) δ ppm 1.26 (3H, t, J=7.3 Hz), 3.06 (2H, q, J=7.3 Hz), 4.97 (2H, q, J=8.9 Hz), 6.43 (1H, d, J=3.4 Hz), 6.98 (1H, d, J=3.4 Hz), 7.17-7.23 (1H, m), 7.37-7.51 (2H, m), 11.89(1H, br. s.).

### Example 263

### 2-(Ethylsulfinyl)-3-[3-fluoro-4-(2,2,2-trifluoroethoxy)phenyl]-3,7-dihydro-4H-pyrrolo[2,3-d]pyrimidin-4-one

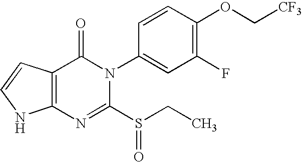

To a methanol (300 ml) solution of 2-(ethylsulfanyl)-3-[3-fluoro-4-(2,2,2-trifluoroethoxy)phenyl]-3,7-dihydro-4H-pyrrolo[2,3-d]pyrimidin-4-one (8.0 g) obtained by the method of Example 262, or a method pursuant to thereto, an aqueous solution (100 ml) of Oxone (registered trademark) monopersulfate compound (12.7 g) was added dropwise at room temperature, and then the mixture was stirred for 30 minutes at 80° C. The reaction mixture was returned to room temperature, and then methanol was distilled off under reduced pressure. Precipitates generated therefrom were collected by filtration, washed with water, dried under reduced pressure, and thus the title compound (7.81 g) was obtained as a pale brown powder.

¹H NMR (300 MHz, DMSO-d₆) δ ppm 1.08 (3H, t, J=7.3 Hz), 2.75-2.89 (1H, m), 2.93-3.10 (1H, m), 4.97 (2H, d, J=9.0 Hz), 6.57-6.62 (1H, m), 7.21-7.74 (4H, m), 12.46 (1H, br. s.).

### Example 264

### 3-[3-Fluoro-4-(2,2,2-trifluoroethoxy)phenyl]-2-(2,2,2-trifluoroethoxy)-3,7-dihydro-4H-pyrrolo[2,3-d]pyrimidin-4-one

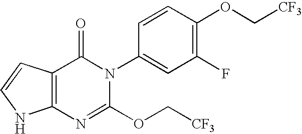

2,2,2-Trifluoroethanol (30 ml) was added dropwise to a mixture of sodium hydride (60% in oil, 562 mg) and tetrahydrofuran (20 ml). To the mixture, 2-(ethylsulfinyl)-3-[3-fluoro-4-(2,2,2-trifluoroethoxy)phenyl]-3,7-dihydro-4H-pyrrolo[2,3-d]pyrimidin-4-one (1900 mg) obtained by the method of Example 263, or a method pursuant to thereto was added. The mixture was stirred for 2 hours at room temperature, and then was concentrated under reduced pressure. Water was added to the residue, and the pH of the mixture was adjusted to about 6 with a 5% aqueous solution of citric acid. Then, the mixture was extracted with ethyl acetate. The organic layer was washed with water and saturated brine, dried over anhydrous sodium sulfate, and then concentrated under reduced pressure. The residue was purified by chromatography, to obtain a pale orange solid (1.90 g). This solid (100 mg) was recrystallized from a mixed solvent of ethyl acetate/hexane. Thus, the title compound (60 mg) was obtained as a pale orange-colored solid.

¹H NMR (300 MHz, DMSO-d₆) δ ppm 4.88-5.03 (4H, m), 6.44 (1H, d, J=3.4 Hz), 6.96 (1H, d, J=3.4 Hz), 7.17 (1H, dq, J=8.7, 1.3 Hz), 7.34-7.46 (2H, m), 11.85 (1H, s).

### Example 265

### 2-(Ethylsulfanyl)-3-[4-(2,2,2-trifluoroethoxy)phenyl]-5,7-dihydro-3H-pyrrolo[2,3-d]pyrimidine-4,6-dione

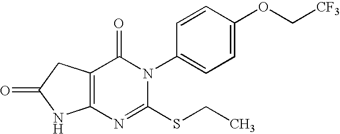

2-(Ethylsulfanyl)-3-[4-(2,2,2-trifluoroethoxy)phenyl]-3,7-dihydro-4H-pyrrolo[2,3-d]pyrimidin-4-one (340 mg) obtained by the method of Example 194, or a method pursuant to thereto was dissolved in 2-methylpropan-2-ol (18 ml), and then water (6 ml) was added thereto. A 2-methylpropan-2-ol solution of bromine (1.73 M, 585 μl) was added thereto dropwise under ice cooling, and the resulting mixture was stirred for 10 minutes at 0° C. A 10% aqueous solution of sodium thiosulfate (10 ml) was added to the reaction mixture solution, and the resulting mixture was stirred for 10 minutes at room temperature. Then, water (5 ml) was added thereto, and the mixture was extracted with ethyl acetate. The extract was washed with saturated brine, dried over anhydrous magnesium sulfate, and then concentrated under reduced pressure. The resulting residue was purified by chromatography, and thus the title compound (61 mg) was obtained as a white solid.

¹H NMR (300 MHz, DMSO-d₆) δ ppm 1.23 (3H, t, J=7.3 Hz), 3.03 (2H, q, J=7.3 Hz), 3.36 (2H, s), 4.86 (2H, q, J=8.8 Hz), 7.19 (2H, d, J=9.1 Hz), 7.30 (2H, d, J=9.1 Hz), 11.08 (1H, s).

### Example 266

### 2-Ethoxy-3-[4-(2,2,2-trifluoroethoxy)phenyl]-5,7-dihydro-3H-pyrrolo[2,3-d]pyrimidine-4,6-dione

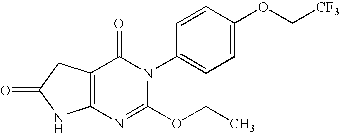

A mixture of 2-ethoxy-3-[4-(2,2,2-trifluoroethoxy)phenyl]-3,7-dihydro-4H-pyrrolo[2,3-d]pyrimidin-4-one (490 mg) obtained by the method of Example 209, or a method pursuant to thereto, iodosobenzene diacetate (600 mg) and acetic acid (20 ml) was stirred for 2 hours at 100° C., and then was concentrated under reduced pressure. Toluene was added to the residue, and the mixture was concentrated again under reduced pressure. The residue was purified by chromatography, and was recrystallized from a mixed solvent of ethyl acetate/hexane. Thus, the title compound (105 mg) was obtained as a pale yellow solid. Furthermore, 2-ethoxy-4-oxo-3-[4-(2,2,2-trifluoroethoxy)phenyl]-4,7-dihydro-3H-pyrrolo[2,3-d]pyrimidin-6-yl acetate (206 mg) of Example 250 was also obtained.

¹H NMR (300 MHz, DMSO-d₆) δ ppm 1.14 (3H, t, J=7.2 Hz), 3.32 (2H, s), 4.32 (2H, d, J=7.2 Hz), 4.83 (2H, d, J=8.7 Hz), 7.13 (2H, d, J=9.1 Hz), 7.23 (2H, d, J=9.1 Hz), 11.05 (1H, s).

### Example 267

### 2-(Methylsulfanyl)-3-[4-(2,2,2-trifluoroethoxy)phenyl]-5,7-dihydro-3H-pyrrolo[2,3-d]pyrimidine-4,6-dione

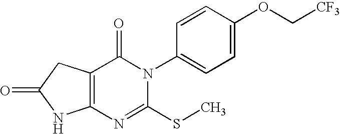

A mixture of 2-(methylsulfanyl)-3-[4-(2,2,2-trifluoroethoxy)phenyl]-3,7-dihydro-4H-pyrrolo[2,3-d]pyrimidin-4-one (1.00 g) obtained by the method of Example 212, or a method pursuant to thereto, iodosobenzene diacetate (1.00 g) and acetic acid (50 ml) was stirred for 2 hours at 100° C. and concentrated under reduced pressure. Toluene was added to the residue, and the mixture was concentrated again under reduced pressure. The residue was purified by chromatography and recrystallized from a mixed solvent of ethyl acetate/hexane to give the title compound (425 mg) as pale brown crystals. Furthermore, 2-(methyl sulfanyl)-4-oxo-3-[4-(2,2,2-trifluoroethoxy)phenyl]-4,7-dihydro-3H-pyrrolo[2,3-d]pyrimidin-6-yl acetate (263 mg) of Example 257 was also obtained.

¹H NMR (300 MHz, DMSO-d₆) δ ppm 2.41 (3H, s), 3.37 (2H, s), 4.86 (2H, q, J=8.9 Hz), 7.19 (2H, d, J=9.0 Hz), 7.31 (2H, d, J=9.0 Hz), 11.10 (1H, s).

### Example 268

### 2-Ethoxy-7-methyl-3-[4-(2,2,2-trifluoroethoxy)phenyl]-5,7-dihydro-3H-pyrrolo[2,3-d]pyrimidine-4,6-dione

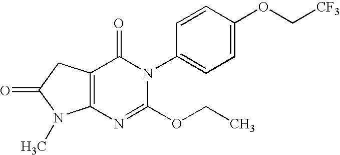

A mixture of 2-ethoxy-7-methyl-3-[4-(2,2,2-trifluoroethoxy)phenyl]-3,7-dihydro-4H-pyrrolo[2,3-d]pyrimidin-4-one (460 mg) obtained in Example 258, iodosobenzene diacetate (403 mg) and acetic acid (30 ml) was stirred for 2 hours at 100° C., and then was concentrated under reduced pressure. Toluene was added to the residue, and the mixture was concentrated again under reduced pressure. The residue was purified by chromatography, and was recrystallized from a mixed solvent of ethyl acetate/hexane. Thus, the title compound (106 mg) was obtained as a pale brown powder. Furthermore, 2-ethoxy-7-methyl-4-oxo-3-[4-(2,2,2-trifluoroethoxy)phenyl]-4,7-dihydro-3H-pyrrolo[2,3-d]pyrimidin-6-yl acetate (75 mg) of Example 259 was also obtained.

¹H NMR (300 MHz, DMSO-d₆) δ ppm 1.18 (3H, t, J=7.0 Hz), 3.10 (3H, s), 3.38 (2H, s), 4.42 (2H, q, J=7.0 Hz), 4.83 (2H, q, J=8.7 Hz), 7.14 (2H, d, J=9.1 Hz), 7.24 (2H, d, J=9.1 Hz).

### Example 269

### 2-(Ethylsulfanyl)-3-[4-(2,2,3,3,3-pentafluoropropoxy)phenyl]-5,7-dihydro-3H-pyrrolo[2,3-d]pyrimidine-4,6-dione

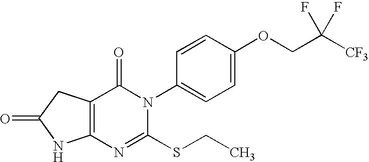

A mixture of 2-(ethylsulfanyl)-3-[4-(2,2,3,3,3-pentafluoropropoxy)phenyl]-3,7-dihydro-4H-pyrrolo[2,3-d]pyrimidin-4-one (1000 mg) obtained by the method of Example 249, or a method pursuant to thereto, iodosobenzene diacetate (844 mg) and acetic acid (20 ml) was stirred for 1.5 hours at 100° C., and then was concentrated under reduced pressure. Toluene was added to the residue, and the mixture was concentrated again under reduced pressure. The residue was purified by chromatography, and was recrystallized from a mixed solvent of ethyl acetate/hexane. Thus, the title compound (296 mg) was obtained as a pale brown solid.

¹H NMR (300 MHz, DMSO-d₆) δ ppm 1.23 (3H, t, J=7.3 Hz), 3.03 (2H, q, J=7.3 Hz), 3.36 (2H, s), 4.93 (2H, t, J=13.3 Hz), 7.20 (2H, d, J=9.1 Hz), 7.30 (2H, d, J=9.1 Hz), 11.08 (1H, s).

### Example 270

### 3-[4-(2,2-Difluoroethoxy)phenyl]-2-(ethylsulfanyl)-5,7-dihydro-3H-pyrrolo[2,3-d]pyrimidine-4,6-dione

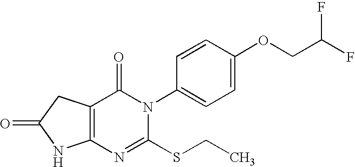

A mixture of 3-[4-(2,2-difluoroethoxy)phenyl]-2-(ethylsulfanyl)-3,7-dihydro-4H-pyrrolo[2,3-d]pyrimidin-4-one (956 mg) obtained by the method of Example 248, or a method pursuant to thereto, iodosobenzene diacetate (876 mg) and acetic acid (40 ml) was stirred for 2 hours at 90° C., and then was concentrated under reduced pressure. Toluene was added to the residue, and the mixture was concentrated again under reduced pressure. The residue was purified by chromatography, and was recrystallized from a mixed solvent of ethyl acetate/hexane. Thus, the title compound (168 mg) was obtained as a pale brown solid.

¹H NMR (300 MHz, DMSO-d₆) δ ppm 1.22 (3H, t, J=7.3 Hz), 3.02 (2H, q, J=7.3 Hz), 3.36 (2H, s), 4.40 (2H, td, J=14.7, 3.5 Hz), 6.43 (1H, tt, J=54.5, 3.5 Hz), 7.14 (2H, d, J=8.7 Hz), 7.26 (2H, d, J=8.7 Hz), 11.06 (1H, s).

### Example 271

### 3-[4-(2,2-Difluoroethoxy)phenyl]-2-(ethylsulfinyl)-5,7-dihydro-3H-pyrrolo[2,3-d]pyrimidine-4,6-dione

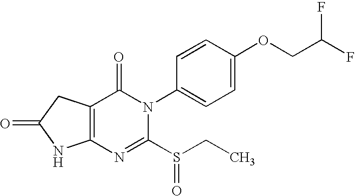

A solution of Oxone (registered trademark) monopersulfate compound (376 mg) in water (10 ml) was added dropwise to a mixture of 3-[4-(2,2-difluoroethoxy)phenyl]-2-(ethylsulfanyl)-5,7-dihydro-3H-pyrrolo[2,3-d]pyrimidine-4,6-dione (225 mg) obtained by the method of Example 270, or a method pursuant to thereto, and methanol (100 ml). The resulting mixture was stirred for one hour at 70° C. and overnight at room temperature, and then was concentrated under reduced pressure. Water was added to the residue, and then the mixture was extracted with ethyl acetate. The organic layer was washed with water and saturated brine, and was dried over anhydrous sodium sulfate, and the solvent was distilled off. Thus, the title compound (234 mg) was obtained as a brown solid.

¹H NMR (300 MHz, DMSO-d₆) δ ppm 1.07 (3H, t, J=7.4 Hz), 2.74 (1H, dq, J=13.8, 7.4 Hz), 2.95 (1H, dd, J=13.8, 7.4 Hz), 3.48 (2H, s), 4.41 (2H, td, J=14.4, 3.4 Hz), 6.43 (1H, tt, J=54.5, 3.4 Hz), 7.02-7.22 (2H, m), 7.36-7.57 (2H, m), 11.43 (1H, s).

### Example 272

### 3-[4-(2,2-Difluoroethoxy)phenyl]-2-(2,2,2-trifluoroethoxy)-5,7-dihydro-3H-pyrrolo[2,3-d]pyrimidine-4,6-dione

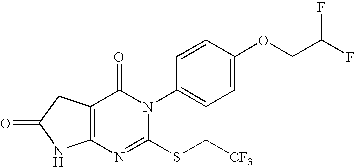

2,2,2-Trifluoroethanol (2 ml) was added to a mixture of sodium hydride (60% in oil, 80 mg) and tetrahydrofuran (10 ml). To the mixture, 3-[4-(2,2-difluoroethoxy)phenyl]-2-(ethylsulfinyl)-5,7-dihydro-3H-pyrrolo[2,3-d]pyrimidine-4,6-dione (234 mg) obtained by the method of Example 271, or a method pursuant to thereto was added, and the resulting mixture was stirred for 15 minutes at room temperature. Then, a 5% aqueous solution of citric acid was added thereto, and the mixture was concentrated under reduced pressure. Water was added to the residue, and the mixture was extracted with ethyl acetate. The organic layer was washed with water and saturated brine, dried over anhydrous sodium sulfate, and then concentrated under reduced pressure. The residue was purified by chromatography, and then was recrystallized from a mixed solvent of ethyl acetate/hexane. Thus, the title compound (71 mg) was obtained as a pale orange-colored solid.

¹H NMR (300 MHz, DMSO-d₆) δ ppm 3.37 (2H, s), 4.38 (2H, td, J=14.7, 3.5 Hz), 4.97 (2H, q, J=9.0 Hz), 6.42 (1H, tt, J=54.5, 3.5 Hz), 7.11 (2H, d, J=9.1 Hz), 7.23 (2H, d, J=9.1 Hz), 11.15 (1H, s).

### Example 273

### 2-(Methylsulfinyl)-3-[4-(2,2,2-trifluoroethoxy)phenyl]-5,7-dihydro-3H-pyrrolo[2,3-d]pyrimidine-4,6-dione

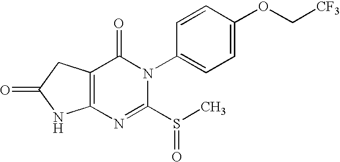

A solution of Oxone (registered trademark) monopersulfate compound (16.9 g) in water (70 ml) was added dropwise to a mixture of 2-(methylsulfanyl)-3-[4-(2,2,2-trifluoroethoxy)phenyl]-5,7-dihydro-3H-pyrrolo[2,3-d]pyrimidine-4,6-dione (9.36 g) and methanol (250 ml) at 50° C. The resulting mixture was stirred for 30 minutes at 50° C., and then was concentrated under reduced pressure. Water was added to the residue, and a precipitated solid was collected by filtration. The solid was washed with water and a mixed solvent of diisopropyl ether/hexane and dried to give a pale purple solid (7.54 g). This pale purple solid (200 mg) was recrystallized from ethyl acetate to give the title compound (115 mg) as a pale red solid.

¹H NMR (300 MHz, DMSO-d₆) δ ppm 2.69 (3H, s), 3.49 (2H, s), 4.86 (2H, q, J=8.8 Hz), 7.13-7.29 (2H, m), 7.37-7.49 (1H, m), 7.49-7.61 (1H, m), 11.44(1H, s).

### Example 274

### 2-(2,2,2-Trifluoroethoxy)-3-[4-(2,2,2-trifluoroethoxy)phenyl]-5,7-dihydro-3H-pyrrolo[2,3-d]pyrimidine-4,6-dione

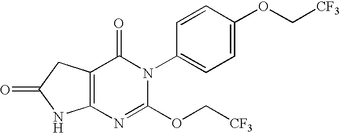

A tetrahydrofuran (3 ml) solution of 2,2,2-trifluoroethanol (1 ml) was added dropwise to a mixture of sodium hydride (60% in oil, 40 mg) and tetrahydrofuran (6 ml). The resultant mixture was stirred for 10 minutes at room temperature. To the mixture, 2-(methylsulfinyl)-3-[4-(2,2,2-trifluoroethoxy)phenyl]-5,7-dihydro-3H-pyrrolo[2,3-d]pyrimidine-4,6-dione (100 mg) was added, and the resulting mixture was stirred for one hour at 60° C., and then was concentrated under reduced pressure. Water and a 5% aqueous solution of citric acid were added to the residue, and the mixture was extracted with ethyl acetate. The organic layer was washed with water and saturated brine, dried over anhydrous sodium sulfate, and then concentrated under reduced pressure. The residue was purified by chromatography, and then was recrystallized from a mixed solvent of ethyl acetate/hexane. Thus, the title compound (55 mg) was obtained as a white solid.

¹H NMR (300 MHz, DMSO-d₆) δ ppm 3.37 (2H, s), 4.84 (2H, q, J=8.9 Hz), 4.97 (2H, q, J=8.7 Hz), 7.16 (2H, d, J=9.0 Hz), 7.26 (2H, d, J=9.0 Hz), 11.17 (1H, s).

### Example 275

### 2-(2,2-Difluoroethoxy)-3-[4-(2,2,2-trifluoroethoxy)phenyl]-5,7-dihydro-3H-pyrrolo[2,3-d]pyrimidine-4,6-dione

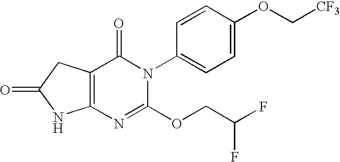

A mixture of 2-(2,2-difluoroethoxy)-3-[4-(2,2,2-trifluoroethoxy)phenyl]-3,7-dihydro-4H-pyrrolo[2,3-d]pyrimidin-4-one (467 mg) obtained by the method of Example 244, or a method pursuant to thereto, iodosobenzene diacetate (419 mg) and acetic acid (20 ml) was stirred for one hour at 100° C., and then was concentrated under reduced pressure. Toluene was added to the residue, and the mixture was concentrated again under reduced pressure. The residue was purified by chromatography, and was recrystallized from a mixed solvent of ethyl acetate/hexane. Thus, the title compound (125 mg) was obtained as a pale orange-colored solid.

¹NMR (300 MHz, DMSO-d₆) δ ppm 3.35 (2H, s), 4.58 (2H, td, J=14.8, 3.2 Hz), 4.83 (2H, q, J=8.9 Hz), 6.23 (1H, tt, J=54.1, 3.2 Hz), 7.14 (2H, d, J=9.0 Hz), 7.25 (2H, d, J=9.0 Hz), 11.12 (1H, s).

### Example 276

### 2-(Ethylsulfinyl)-3-[4-(2,2,3,3,3-pentafluoropropoxy)phenyl]-5,7-dihydro-3H-pyrrolo[2,3-d]pyrimidine-4,6-dione

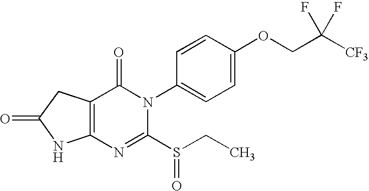

A solution of Oxone (registered trademark) monopersulfate compound (424 mg) in water (2 ml) was added dropwise to a mixture of 2-(ethylsulfanyl)-3-[4-(2,2,3,3,3-pentafluoropropoxy)phenyl]-5,7-dihydro-3H-pyrrolo[2,3-d]pyrimidine-4,6-dione (290 mg) obtained by the method of Example 269, or a method pursuant to thereto, and methanol (10 ml) at 60° C. The resulting mixture was stirred for 30 minutes at 60° C., and was cooled to room temperature. Then, water was added thereto, and the mixture was extracted with ethyl acetate. The organic layer was washed with water and saturated brine, and was dried over anhydrous sodium sulfate, and the solvent was distilled off. Thus, the title compound (200 mg) was obtained as a yellow solid.

¹H NMR (300 MHz, DMSO-d₆) δ ppm 1.07 (3H, t, J=7.4 Hz), 2.66-2.85 (1H, m), 2.86-3.03 (1H, m), 3.48 (2H, s), 4.94 (2H, t, J=13.3 Hz), 7.17-7.29 (2H, m), 7.37-7.49 (1H, m), 7.51-7.65 (1H, m), 11.44 (1H, s).

### Example 277

### 2-Ethoxy-3-[4-(2,2,3,3,3-pentafluoropropoxy)phenyl]-5,7-dihydro-3H-pyrrolo[2,3-d]pyrimidine-4,6-dione

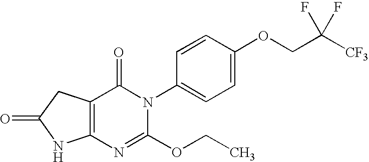

A mixture of 2-(ethylsulfinyl)-3-[4-(2,2,3,3,3-pentafluoropropoxy)phenyl]-5,7-dihydro-3H-pyrrolo[2,3-d]pyrimidine-4,6-dione (248 mg) obtained by the method of Example 276, or a method pursuant to thereto, a 20% sodium ethoxide-ethanol solution (1 ml), ethanol (10 ml) and tetrahydrofuran (20 ml) was stirred for 30 minutes at 60° C., and then was concentrated under reduced pressure. Water and a 5% aqueous solution of citric acid were added to the residue, and the mixture was extracted with ethyl acetate. The organic layer was washed with water and saturated brine, dried over anhydrous sodium sulfate, and then concentrated under reduced pressure. The residue was purified by chromatography, and then was recrystallized from a mixed solvent of ethyl acetate/hexane. Thus, the title compound (30 mg) was obtained as a white solid.

¹H NMR (300 MHz, DMSO-d₆) δ ppm 1.14 (3H, t, J=7.0 Hz), 3.32 (2H, s), 4.33 (2H, q, J=7.0 Hz), 4.90 (2H, t, J=13.4 Hz), 7.14 (2H, d, J=9.0 Hz), 7.24(2H, d, J=9.0 Hz), 11.05 (1H, s).

### Example 278

### 2-(3-Ethoxypropoxy)-3-[4-(2,2,2-trifluoroethoxy)phenyl]-5,7-dihydro-3H-pyrrolo[2,3-d]pyrimidine-4,6-dione

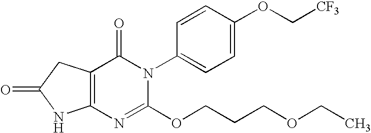

A mixture of 2-(3-ethoxypropoxy)-3-[4-(2,2,2-trifluoroethoxy)phenyl]-3,7-dihydro-4H-pyrrolo[2,3-d]pyrimidin-4-one (480 mg) obtained by the method of Example 261, or a method pursuant to thereto, iodosobenzene diacetate (376 mg) and acetic acid (15 ml) was stirred for 2 hours at 100° C. The reaction mixture was returned to room temperature, and then the solvent was distilled off under reduced pressure. The resulting residue was purified by chromatography, and then was recrystallized from a mixed solvent of ethyl acetate/hexane. Thus, the title compound (101 mg) was obtained as a white powder.

¹H NMR (300 MHz, DMSO-d₆) δ ppm 1.03 (3H, t, J=7.0 Hz), 1.67-1.80 (2H, m), 3.21 (2H, t, J=6.2 Hz), 3.24-3.31 (2H, m), 3.32 (2H, s), 4.32 (2H, t, J=6.2 Hz), 4.82 (2H, q, J=9.0 Hz), 7.14 (2H, d, J=9.0 Hz), 7.24 (2H, d, J=9.0 Hz), 11.04 (1H, s).

### Example 279

### 2-(1-Methylethoxy)-3-[4-(2,2,2-trifluoroethoxy)phenyl]-3,7-dihydro-4H-pyrrolo[2,3-d]pyrimidin-4-one

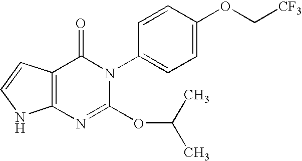

Sodium hydride (60% in oil, 240 mg) was added to a solution of propan-2-ol (360 μl) in N,N-dimethylformamide (15 ml), and the resulting mixture was stirred for 30 minutes at room temperature.

2-(Methylsulfinyl)-3-[4-(2,2,2-trifluoroethoxy)phenyl]-3,7-dihydro-4H-pyrrolo[2,3-d]pyrimidin-4-one (794 mg) obtained by the method of Example 260, or a method pursuant to thereto was added to this mixture, and the resulting mixture was stirred for one hour at 80° C. The reaction mixture was returned to room temperature, and then the solvent was distilled off under reduced pressure. The resulting residue was diluted with ethyl acetate, and the dilution was washed with 0.1 M hydrochloric acid and saturated brine, dried over anhydrous magnesium sulfate, and then concentrated under reduced pressure. The resulting residue was purified by chromatography, and thus the title compound (483 mg) was obtained as a white powder.

¹H NMR (300 MHz, DMSO-d₆) δ ppm 1.17 (6H, d, J=6.1 Hz), 4.83 (2H, q, J=8.7 Hz), 5.11-5.23 (1H, m), 6.37 (1H, d, J=3.4 Hz), 6.87 (1H, d, J=3.4 Hz), 7.13 (2H, d, J=9.0 Hz), 7.20 (2H, d, J=9.0 Hz), 11.65 (1H, br. s.).

### Example 280

### 2-(1-Methylethoxy)-3-[4-(2,2,2-trifluoroethoxy)phenyl]-5,7-dihydro-3H-pyrrolo[2,3-d]pyrimidine-4,6-dione

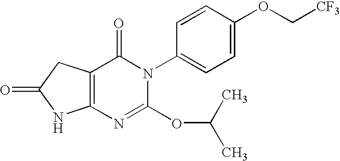

A mixture of 2-(1-methylethoxy)-3-[4-(2,2,2-trifluoroethoxy)phenyl]-3,7-dihydro-4H-pyrrolo[2,3-d]pyrimidin-4-one (450 mg) obtained by the method of Example 279, or a method pursuant to thereto, iodosobenzene diacetate (395 mg) and acetic acid (10 ml) was stirred for 2 hours at 100° C. The reaction mixture was returned to room temperature, and then the solvent was distilled off under reduced pressure. The resulting residue was purified by chromatography, and then was recrystallized from a mixed solvent of ethyl acetate/hexane. Thus, the title compound (99 mg) was obtained as a white powder.

¹H NMR (300 MHz, DMSO-d₆) δ ppm 1.16 (6H, d, J=6.2 Hz), 3.31 (2H, s), 4.83 (2H, q, J=8.9 Hz), 5.11-5.25 (1H, m), 7.13 (2H, d, J=9.0 Hz), 7.21 (2H, d, J=9.0 Hz), 11.02 (1H, s).

### Example 281

### 2-Propoxy-3-[4-(2,2,2-trifluoroethoxy)phenyl]-3,7-dihydro-4H-pyrrolo[2,3-d]pyrimidin-4-one

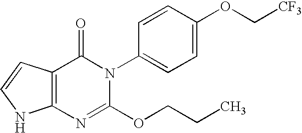

Sodium hydride (60% in oil, 240 mg) was added to a solution of propan-1-ol (360 μl) in N,N-dimethylformamide (15 ml), and the resulting mixture was stirred for 30 minutes at room temperature. A N,N-dimethylformamide (10 ml) solution of 2-(methylsulfinyl)-3-[4-(2,2,2-trifluoroethoxy)phenyl]-3,7-dihydro-4H-pyrrolo[2,3-d]pyrimidin-4-one (794 mg) obtained by the method of Example 260, or a method pursuant to thereto was added to this mixture, and the resulting mixture was stirred for one hour at 80° C. The reaction mixture was returned to room temperature, and then the solvent was distilled off under reduced pressure. The resulting residue was diluted with ethyl acetate, and the dilution was washed with 0.1 M hydrochloric acid and saturated brine, dried over anhydrous magnesium sulfate, and then concentrated under reduced pressure. The resulting residue was purified by chromatography, and thus the title compound (505 mg) was obtained as a white powder.

¹H NMR (400 MHz, DMSO-d₆) δ ppm 0.74 (3H, t, J=7.3 Hz), 1.48-1.58 (2H, m), 4.20 (2H, t, J=6.4 Hz), 4.84 (2H, q, J=9.0 Hz), 6.37 (1H, dd, J=3.2, 2.1 Hz), 6.88 (1H, dd, J=3.2, 2.1 Hz), 7.14 (2H, d, J=9.2 Hz), 7.23 (2H, d, J=9.2 Hz), 11.69 (1H, br. s.).

### Example 282

### 2-Propoxy-3-[4-(2,2,2-trifluoroethoxy)phenyl]-5,7-dihydro-3H-pyrrolo[2,3-d]pyrimidine-4,6-dione

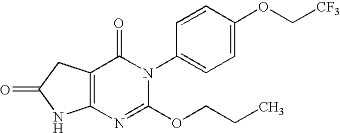

A mixture of 2-propoxy-3-[4-(2,2,2-trifluoroethoxy)phenyl]-3,7-dihydro-4H-pyrrolo[2,3-d]pyrimidin-4-one (500 mg) obtained by the method of Example 281, or a method pursuant to thereto, iodosobenzene diacetate (422 mg) and acetic acid (15 ml) was stirred for 2 hours at 100° C. The reaction mixture was returned to room temperature, and then the solvent was distilled off under reduced pressure. The resulting residue was purified by chromatography, and then was recrystallized from a mixed solvent of ethyl acetate/hexane. Thus, the title compound (87 mg) was obtained as a white powder.

¹H NMR (300 MHz, DMSO-d₆) δ ppm 0.73 (3H, t, J=7.4 Hz), 1.44-1.61 (2H, m), 3.32 (2H, s), 4.22 (2H, t, J=6.2 Hz), 4.83 (2H, q, J=8.8 Hz), 7.14 (2H, d, J=9.0 Hz), 7.23 (2H, d, J=9.0 Hz), 11.03 (1H, br. s.).

### Example 283

### 2-(Cyclobutyloxy)-3-[4-(2,2,2-trifluoroethoxy)phenyl]-3,7-dihydro-4H-pyrrolo[2,3-d]pyrimidin-4-one

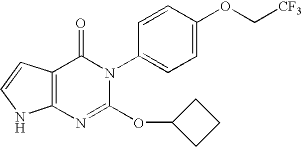

To a solution of cyclobutanol (433 mg) in N,N-dimethylformamide (15 ml), sodium hydride (60% in oil, 240 mg) was added. The resulting mixture was stirred for 30 minutes at room temperature. To this mixture, a solution of 2-(methylsulfinyl)-3-[4-(2,2,2-trifluoroethoxy)phenyl]-3,7-dihydro-4H-pyrrolo[2,3-d]pyrimidin-4-one (794 mg) obtained by the method of Example 260, or a method pursuant to thereto in N,N-dimethylformamide (10 ml) was added, and the resulting mixture was stirred for 30 minutes at 80° C. The reaction mixture was returned to room temperature, and then the solvent was distilled off under reduced pressure. The resulting residue was diluted with ethyl acetate, and the dilution was washed with 1 M hydrochloric acid and saturated brine, dried over anhydrous magnesium sulfate, and then concentrated under reduced pressure. The resulting residue was purified by chromatography, and thus the title compound (580 mg) was obtained as a white powder.

¹H NMR (300 MHz, DMSO-d₆) δ ppm 1.47-1.75 (2H, m), 1.78-1.95 (2H, m), 2.24-2.39 (2H, m), 4.83 (2H, q, J=8.7 Hz), 5.01-5.14 (1H, m), 6.36 (1H, d, J=3.4 Hz), 6.87 (1H, d, J=3.4 Hz), 7.14 (2H, d, J=8.7 Hz), 7.24 (2H, d, J=8.7 Hz), 11.64 (1H, br. s.).

### Example 284

### 2-(Cyclobutyloxy)-3-[4-(2,2,2-trifluoroethoxy)phenyl]-5,7-dihydro-3H-pyrrolo[2,3-d]pyrimidine-4,6-dione

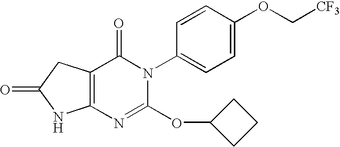

A mixture of 2-(cyclobutyloxy)-3-[4-(2,2,2-trifluoroethoxy)phenyl]-3,7-dihydro-4H-pyrrolo[2,3-d]pyrimidin-4-one (550 mg) obtained by the method of Example 283, or a method pursuant to thereto, iodosobenzene diacetate (607 mg) and acetic acid (15 ml) was stirred for 2 hours at 100° C. The reaction mixture was returned to room temperature, and then the solvent was distilled off under reduced pressure. The resulting residue was purified by chromatography, and then was recrystallized from a mixed solvent of ethyl acetate/hexane. Thus, the title compound (118 mg) was obtained as a white powder.

¹H NMR (400 MHz, DMSO-d₆) δ ppm 1.48-1.62 (1H, m), 1.63-1.74 (1H, m), 1.81-1.95 (2H, m), 2.21-2.34 (2H, m), 3.31 (2H, s), 4.84 (2H, q, J=9.0 Hz), 5.01-5.12 (1H, m), 7.14 (2H, d, J=9.1 Hz), 7.25 (2H, d, J=9.0 Hz), 11.02 (1H, s).

### Example 285

### 2-[(1-Methylethyl)sulfanyl]-3-[4-(2,2,2-trifluoroethoxy)phenyl]-5,7-dihydro-3H-pyrrolo[2,3-d]pyrimidine-4,6-dione

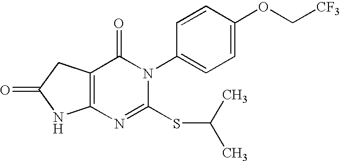

A mixture of 2-thioxo-3-[4-(2,2,2-trifluoroethoxy)phenyl]-2,3,5,7-tetrahydro-1H-pyrrolo[2,3-d]pyrimidine-4,6-dione (200 mg) obtained by the method of Example 46, or a method pursuant to thereto, a 1 M aqueous solution of sodium hydrogen carbonate (560 μl), 2-iodopropane (280 μl) and acetonitrile (5.5 ml) was heated to reflux for one hour. The reaction mixture was returned to room temperature, and then was diluted with ethyl acetate (50 ml). The dilution was washed with saturated brine, dried over anhydrous magnesium sulfate, and then concentrated under reduced pressure. The resulting residue was purified by chromatography, and thus the title compound (215 mg) was obtained as a white solid.

¹H NMR (300 MHz, DMSO-d₆) δ ppm 1.29 (6H, d, J=6.8 Hz), 3.36 (2H, s), 3.80 (1H, spt, J=6.8 Hz), 4.86 (2H, q, J=8.8 Hz), 7.18 (2H, d, J=9.1 Hz), 7.28 (2H, d, J=9.1 Hz), 11.07 (1H, s).

### Example 286 2-(Propylsulfanyl)-3-[4-(2,2,2-trifluoroethoxy)phenyl]-5,7-dihydro-3H-pyrrolo[2,3-d]pyrimidine-4,6-dione

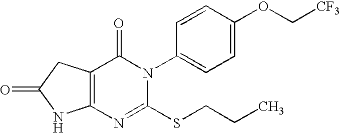

A mixture of 2-thioxo-3-[4-(2,2,2-trifluoroethoxy)phenyl]-2,3,5,7-tetrahydro-1H-pyrrolo[2,3-d]pyrimidine-4,6-dione (200 mg) obtained by the method of Example 46, or a method pursuant to thereto, a 1 M aqueous solution of sodium hydrogen carbonate (560 μl), 1-iodopropane (273 μl) and acetonitrile (5.5 ml) was heated to reflux for one hour. The reaction mixture was returned to room temperature, and then was diluted with ethyl acetate (50 ml). The dilution was washed with saturated brine, dried over anhydrous magnesium sulfate, and then concentrated under reduced pressure. The resulting residue was purified by chromatography, and thus the title compound (209 mg) was obtained as a white solid.

¹H NMR (300 MHz, DMSO-d₆) δ ppm 0.90 (3H, t, J=7.4 Hz), 1.60 (2H, qt, J=7.4, 7.2 Hz), 3.02 (2H, t, J=7.2 Hz), 3.36 (2H, s), 4.86 (2H, q, J=9.0 Hz), 7.19 (2H, d, J=9.1 Hz), 7.30 (2H, d, J=9.1 Hz), 11.05 (1H, s).

### Example 287

### 2-Butoxy-3-[4-(2,2,2-trifluoroethoxy)phenyl]-3,7-dihydro-4H-pyrrolo[2,3-d]pyrimidin-4-one

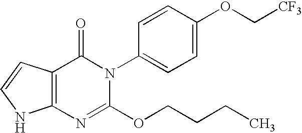

To a solution of butan-1-ol (444 mg) in N,N-dimethylformamide (15 ml), sodium hydride (60% in oil, 240 mg) was added. The resulting mixture was stirred for 30 minutes at room temperature. To this mixture, a N,N-dimethylformamide (10 ml) solution of 2-(methylsulfinyl)-3-[4-(2,2,2-trifluoroethoxy)phenyl]-3,7-dihydro-4H-pyrrolo[2,3-d]pyrimidin-4-one (794 mg) obtained by the method of Example 260, or a method pursuant to thereto was added, and the resulting mixture was stirred for one hour at 80° C. The reaction mixture was returned to room temperature, and then the solvent was distilled off under reduced pressure. The resulting residue was diluted with ethyl acetate, and the dilution was washed with 0.1 M hydrochloric acid and saturated brine, dried over anhydrous magnesium sulfate, and then concentrated under reduced pressure. The resulting residue was purified by chromatography, and thus the title compound (615 mg) was obtained as a white powder.

¹H NMR (400 MHz, DMSO-d₆) δ ppm 0.79 (3H, t, J=7.5 Hz), 1.11-1.23 (2H, m), 1.45-1.55 (2H, m), 4.24 (2H, t, J=6.4 Hz), 4.84 (2H, q, J=9.0 Hz), 6.37 (1H, dd, J=3.3, 2.2 Hz), 6.88 (1H, dd, J=3.3, 2.2 Hz), 7.14 (2H, d, J=8.8 Hz), 7.22 (2H, d, J=8.8 Hz), 11.69 (1H, br. s.).

### Example 288

### 2-Butoxy-3-[4-(2,2,2-trifluoroethoxy)phenyl]-5,7-dihydro-3H-pyrrolo[2,3-d]pyrimidine-4,6-dione

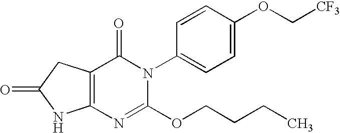

A mixture of 2-butoxy-3-[4-(2,2,2-trifluoroethoxy)phenyl]-3,7-dihydro-4H-pyrrolo[2,3-d]pyrimidin-4-one (600 mg) obtained by the method of Example 287, or a method pursuant to thereto, iodosobenzene diacetate (760 mg) and acetic acid (15 ml) was stirred for 2 hours at 100° C. The reaction mixture was returned to room temperature, and then the solvent was distilled off under reduced pressure. The resulting residue was purified by chromatography, and then was recrystallized from a mixed solvent of ethyl acetate/hexane. Thus, the title compound (110 mg) was obtained as a white powder.

¹H NMR (300 MHz, DMSO-d₆) δ ppm 0.78 (3H, t, J=7.4 Hz), 1.06-1.26 (2H, m), 1.43-1.57 (2H, m), 3.31 (2H, s), 4.27 (2H, t, J=6.4 Hz), 4.83 (2H, q, J=9.0 Hz), 7.13 (2H, d, J=9.0 Hz), 7.23 (2H, d, J=9.0 Hz), 11.03 (1H, s).

### Example 289

### 2-(Cyclopropylmethoxy)-3-[4-(2,2,2-trifluoroethoxy)phenyl]-3,7-dihydro-4H-pyrrolo[2,3-d]pyrimidin-4-one

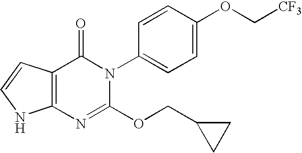

To a solution of cyclopropylmethanol (433 mg) in N,N-dimethylformamide (15 ml), sodium hydride (60% in oil, 240 mg) was added. The resulting mixture was stirred for 30 minutes at room temperature. To this mixture, a solution of 2-(methylsulfinyl)-3-[4-(2,2,2-trifluoroethoxy)phenyl]-3,7-dihydro-4H-pyrrolo[2,3-d]pyrimidin-4-one (794 mg) obtained by the method of Example 260, or a method pursuant to thereto in N,N-dimethylformamide (10 ml) was added, and the resulting mixture was stirred for one hour at 80° C. The reaction mixture was returned to room temperature, and then the solvent was distilled off under reduced pressure. The resulting residue was diluted with ethyl acetate, and the dilution was washed with 1 M hydrochloric acid and saturated brine, dried over anhydrous magnesium sulfate, and then concentrated under reduced pressure. The resulting residue was purified by chromatography, and thus the title compound (580 mg) was obtained as a white powder.

¹H NMR (300 MHz, DMSO-d₆) δ ppm 0.15-0.23 (2H, m), 0.38-0.48 (2H, m), 1.01-1.16 (1H, m), 4.12 (2H, d, J=7.0 Hz), 4.84 (2H, q, J=8.9 Hz), 6.37 (1H, dd, J=3.2, 2.1 Hz), 6.88 (1H, dd, J=3.2, 2.1 Hz), 7.14 (2H, d, J=9.0 Hz), 7.23 (2H, d, J=9.0 Hz), 11.66 (1H, br. s.).

### Example 290

### 2-(Cyclopropylmethoxy)-3-[4-(2,2,2-trifluoroethoxy)phenyl]-5,7-dihydro-3H-pyrrolo[2,3-d]pyrimidine-4,6-dione

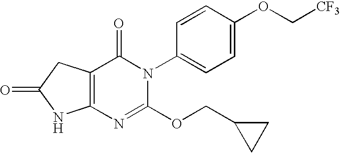

To a mixture of 2-(cyclopropylmethoxy)-3-[4-(2,2,2-trifluoroethoxy)phenyl]-3,7-dihydro-4H-pyrrolo[2,3-d]pyrimidin-4-one (550 mg) obtained by the method of Example 289, or a method pursuant to thereto, 2-methylpropan-2-ol (15 ml) and water (5 ml), a 2-methylpropan-2-ol solution of bromine (2.33 M, 0.62 ml), which had been prepared previously, was added dropwise under ice cooling. The mixture was stirred for 30 minutes at room temperature, and then a 10% aqueous solution of sodium thiosulfate was added thereto. The mixture was stirred for 10 minutes, and was extracted with ethyl acetate. The organic layer was washed with saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (86 mg) was obtained as a white powder.

¹H NMR (300 MHz, DMSO-d₆) δ ppm 0.17-0.23 (2H, m), 0.41-0.48 (2H, m), 1.00-1.14 (1H, m), 3.32 (2H, s), 4.14 (2H, d, J=7.0 Hz), 4.83 (2H, q, J=8.9 Hz), 7.15 (2H, d, J=9.0 Hz), 7.24 (2H, d, J=9.0 Hz), 11.02 (1H, s).

### Example 291

### 2-(Cyclopropylsulfanyl)-3-[4-(2,2,2-trifluoroethoxy)phenyl]-5,7-dihydro-3H-pyrrolo[2,3-d]pyrimidine-4,6-dione

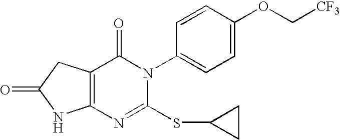

To a mixture of 2-(cyclopropylsulfanyl)-3-[4-(2,2,2-trifluoroethoxy)phenyl]-3,7-dihydro-4H-pyrrolo[2,3-d]pyrimidin-4-one (300 mg) obtained by the method of Example 256, or a method pursuant to thereto, 2-methylpropan-2-ol (27 ml) and water (9 ml), a 2-methylpropan-2-ol solution of bromine (3.74 M, 0.21 ml), which had been prepared previously, was added dropwise under ice cooling. The mixture was stirred for 30 minutes at room temperature, and then a 10% aqueous solution of sodium thiosulfate was added thereto. The mixture was stirred for 10 minutes, and was extracted with ethyl acetate. The organic layer was washed with saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (16 mg) was obtained as a white powder.

^{I}H NMR (300 MHz, DMSO-d₆) δ ppm 0.54-0.61 (2H, m), 0.97-1.05 (2H, m), 2.19-2.31 (1H, m), 3.36 (2H, s), 4.85 (2H, q, J=8.9 Hz), 7.17 (2H, d, J=9.0 Hz), 7.29 (2H, d, J=9.0 Hz), 11.12 (1H, s).

### Example 292

### 2-(4-Fluorophenoxy)-3-[4-(2,2,2-trifluoroethoxy)phenyl]-5,7-dihydro-3H-pyrrolo[2,3-d]pyrimidine-4,6-dione

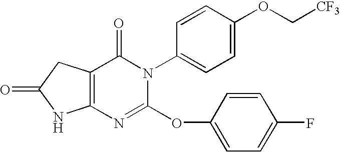

A solution of 4-fluorophenol (112 mg) in N,N-dimethylformamide (3 ml) was added dropwise to a mixture of sodium hydride (60% in oil, 40 mg) and N,N-dimethylformamide (3 ml). To the mixture, a solution of 2-(methylsulfinyl)-3-[4-(2,2,2-trifluoroethoxy)phenyl]-5,7-dihydro-3H-pyrrolo[2,3-d]pyrimidine-4,6-dione (200 mg) in N,N-dimethylformamide (10 ml) was added, and the resulting mixture was stirred for 30 minutes at room temperature. Then, water and a 5% aqueous solution of citric acid were added in an ice bath, and then the mixture was extracted with ethyl acetate. The organic layer was washed with water and saturated brine, dried over anhydrous sodium sulfate, and then concentrated under reduced pressure. The residue was purified by chromatography, and then was recrystallized from a mixed solvent of ethyl acetate/hexane. Thus, the title compound (25 mg) was obtained as a white solid.

¹H NMR (300 MHz, DMSO-d₆) δ ppm 3.35 (2H, s), 4.83 (2H, q, J=8.7 Hz), 7.18 (2H, d, J=9.1 Hz), 7.22-7.34 (4H, m), 7.47 (2H, d, J=9.1 Hz), 11.01 (1H, s).

### Example 293

### 2-Propyl-3-[4-(2,2,2-trifluoroethoxy)phenyl]-5,7-dihydro-3H-pyrrolo[2,3-d]pyrimidine-4,6-dione

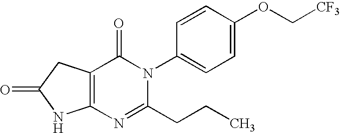

A mixture of ethyl 5-oxo-2-{[1-{[4-(2,2,2-trifluoroethoxy)phenyl]amino}butylidene]amino}-4,5-dihydro-1H-pyrrol-3-carboxylate (21 mg) obtained in Reference Example 50, 4-methylbenzene sulfonic acid (5 mg) and toluene (5 ml) was heated to reflux for one day, and then diphosphorus pentoxide (about 2 g) was added thereto. The mixture was heated to reflux for one day, cooled to room temperature, and then purified by chromatography. Thus, the title compound (8.5 mg) was obtained as a white solid.

¹H NMR (300 MHz, DMSO-d₆) δ ppm 0.78 (3H, t, J=7.4 Hz), 1.57 (2H, sxt, J=7.4 Hz), 2.27 (2H, t, J=7.4 Hz), 3.37 (2H, s), 4.85 (2H, q, J=9.0 Hz), 7.19 (2H, d, J=8.9 Hz), 7.30 (2H, d, J=8.9 Hz), 11.03 (1H, s).

### Example 294

### 2-[(2-{[Tert-butyl(dimethyl)silyl]oxy}ethyl)sulfanyl]-3-[4-(2,2,2-trifluoroethoxy)phenyl]-5,7-dihydro-3H-pyrrolo[2,3-d]pyrimidine-4,6-dione

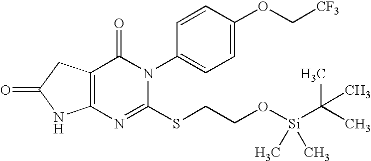

A mixture of 2-thioxo-3-[4-(2,2,2-trifluoroethoxy)phenyl]-2,3,5,7-tetrahydro-1H-pyrrolo[2,3-d]pyrimidine-4,6-dione (200 mg) obtained by the method of Example 46, or a method pursuant to thereto, a 1 M aqueous solution of sodium hydrogen carbonate (560 μl), (2-bromoethoxy)(tert-butyl)dimethylsilane (156 μl) and acetonitrile (5.5 ml) was heated to reflux for 2 hours. The mixture was returned to room temperature, and then was diluted with ethyl acetate (50 ml). The dilution was washed with saturated brine, dried over anhydrous magnesium sulfate, and then concentrated under reduced pressure. The resulting residue was purified by chromatography, and thus the title compound (268 mg) was obtained as a white solid.

¹H NMR (300 MHz, DMSO-d₆) δ ppm 0.01 (6H, s), 0.81 (9H, s), 3.20 (2H, t, J=6.3 Hz), 3.36 (2H, s), 3.76 (2H, t, J=6.3 Hz), 4.85 (2H, q, J=8.9 Hz), 7.19 (2H, d, J=9.1 Hz), 7.28 (2H, d, J=9.1 Hz), 11.06 (1H, s).

### Example 295

### 2-[(2-Hydroxyethyl)sulfanyl]-3-[4-(2,2,2-trifluoroethoxy)phenyl]-5,7-dihydro-3H-pyrrolo[2,3-d]pyrimidine-4,6-dione

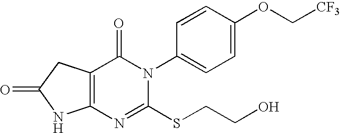

2-[(2-{[Tert-butyl(dimethyl)silyl]oxy}ethyl)sulfanyl]-3-[4-(2,2,2-trifluoroethoxy)phenyl]-5,7-dihydro-3H-pyrrolo[2,3-d]pyrimidine-4,6-dione (261 mg) obtained by the method of Example 294, or a method pursuant to thereto was dissolved in tetrahydrofuran (5 ml), and tetrabutylammonium fluoride (1 M tetrahydrofuran solution, 557 μl) was added thereto. The resulting mixture was stirred for 30 minutes at room temperature. The reaction solution was diluted with ethyl acetate (50 ml). The dilution was washed with saturated brine, dried over anhydrous magnesium sulfate, and then concentrated under reduced pressure. The resulting residue was purified by chromatography, and thus the title compound (75.4 mg) was obtained as a yellowish white solid.

¹H NMR (400 MHz, DMSO-d₆) δ ppm 3.15 (2H, t, J=6.3 Hz), 3.36 (2H, s), 3.59 (2H, td, J=6.3, 5.3 Hz), 4.87 (2H, q, J=8.9 Hz), 4.96 (1H, t, J=5.3 Hz), 7.19 (2H, d, J=9.1 Hz), 7.31 (2H, d, J=9.1 Hz), 11.09 (1H, s).

### Example 296

### 2-[(Cyclobutylmethyl)sulfanyl]-3-[4-(2,2,2-trifluoroethoxy)phenyl]-5,7-dihydro-3H-pyrrolo[2,3-d]pyrimidine-4,6-dione

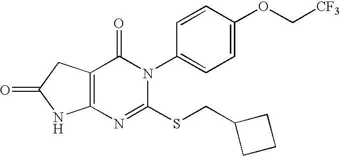

A mixture of 2-thioxo-3-[4-(2,2,2-trifluoroethoxy)phenyl]-2,3,5,7-tetrahydro-1H-pyrrolo[2,3-d]pyrimidine-4,6-dione (200 mg) obtained by the method of Example 46, or a method pursuant to thereto, a 1 M aqueous solution of sodium hydrogen carbonate (560 μl), (bromomethyl)cyclobutane (311 μl) and acetonitrile (5.5 ml) was heated to reflux for 30 minutes. The reaction mixture was returned to room temperature, and then was diluted with ethyl acetate (50 ml). The dilution was washed with saturated brine, dried over anhydrous magnesium sulfate, and then concentrated under reduced pressure. The resulting residue was purified by chromatography, and thus the title compound (207 mg) was obtained as a white solid.

¹H NMR (400 MHz, DMSO-d₆) δ ppm 1.57-1.70 (2H, m), 1.72-1.84 (2H, m), 1.91-2.09 (2H, m), 2.47-2.58 (1H, m), 3.14 (2H, d, J=7.8 Hz), 3.36(2H, s), 4.86(2H, q, J=8.8 Hz), 7.18 (2H, d, J=9.1 Hz), 7.29 (2H, d, J=9.1 Hz), 11.07 (1H, s).

### Example 297

### 2-[(Cyclopropylmethyl)sulfanyl]-3-[4-(2,2,2-trifluoroethoxy)phenyl]-5,7-dihydro-3H-pyrrolo[2,3-d]pyrimidine-4,6-dione

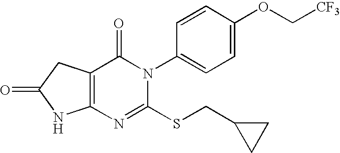

A mixture of 2-thioxo-3-[4-(2,2,2-trifluoroethoxy)phenyl]-2,3,5,7-tetrahydro-1H-pyrrolo[2,3-d]pyrimidine-4,6-dione (200 mg) obtained by the method of Example 46, or a method pursuant to thereto, a 1 M aqueous solution of sodium hydrogen carbonate (560 μl), (bromomethyl)cyclopropane (274 μl) and acetonitrile (5.5 ml) was heated to reflux for 30 minutes. The reaction mixture was cooled to room temperature, and then was diluted with ethyl acetate (50 ml). The dilution was washed with saturated brine, dried over anhydrous magnesium sulfate, and then concentrated under reduced pressure. The resulting residue was purified by chromatography, and thus the title compound (208 mg) was obtained as a white solid.

¹H NMR (400 MHz, DMSO-d₆) δ ppm 0.18-0.30 (2H, m), 0.47-0.55 (2H, m), 0.96-1.18 (1H, m), 2.99 (2H, d, J=7.3 Hz), 3.36 (2H, s), 4.87 (2H, q, J=8.8 Hz), 7.20 (2H, d, J=9.0 Hz), 7.31 (2H, d, J=9.0 Hz), 11.08 (1H, s).

### Example 298

### 3-[4-(2,2,2-Trifluoroethoxy)phenyl]-2-[(2,2,2-trifluoroethyl)sulfanyl]-5,7-dihydro-3H-pyrrolo[2,3-d]pyrimidine-4,6-dione

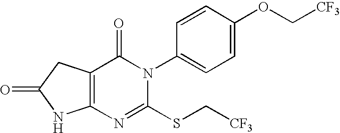

A mixture of 2-thioxo-3-[4-(2,2,2-trifluoroethoxy)phenyl]-2,3,5,7-tetrahydro-1H-pyrrolo[2,3-d]pyrimidine-4,6-dione (1 g) obtained by the method of Example 46, or a method pursuant to thereto, 1,1,1-trifluoro-2-iodoethane (1.38 ml), a 1 M aqueous solution of sodium hydrogen carbonate (2.8 ml) and acetonitrile (28 ml) was heated to reflux for 3 hours. The reaction mixture was returned to room temperature, and then was concentrated under reduced pressure. The residue was dissolved in N,N-dimethylformamide (5 ml), and 1,1,1-trifluoro-2-iodoethane (1.38 ml) was added again thereto. The mixture was stirred for 10 minutes at 100° C. The reaction solution was returned to room temperature, and then was concentrated under reduced pressure. The residue was dissolved in ethyl acetate (150 ml), washed with water and saturated brine, dried over anhydrous magnesium sulfate, and then concentrated under reduced pressure. The resulting residue was purified by chromatography, and was recrystallized from a mixed solvent of ethyl acetate/hexane. Thus, the title compound (315 mg) was obtained as a white solid.

¹-H NMR (400 MHz, DMSO-d₆) δ ppm 3.40 (2H, s), 4.18 (2H, q, J=10.3 Hz), 4.88 (2H, q, J=8.8 Hz), 7.23 (2H, d, J=9.0 Hz), 7.38 (2H, d, J=9.0 Hz), 11.18 (1H, s).

### Example 299

### 2-{[3-(2-Methoxyethoxy)propyl]sulfanyl}-3-[4-(2,2,2-trifluoroethoxy)phenyl]-5,7-dihydro-3H-pyrrolo[2,3-d]pyrimidine-4,6-dione

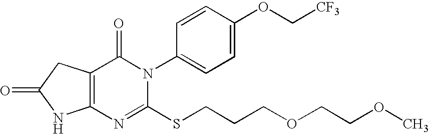

A mixture of 2-thioxo-3-[4-(2,2,2-trifluoroethoxy)phenyl]-2,3,5,7-tetrahydro-1H-pyrrolo[2,3-d]pyrimidine-4,6-dione (200 mg) obtained by the method of Example 46, or a method pursuant to thereto, a 1 M aqueous solution of sodium hydrogen carbonate (560 μl), 1-bromo-3-(2-methoxyethoxy)propane (165 mg) and acetonitrile (5.5 ml) was heated to reflux for one hour. The reaction mixture was returned to room temperature, and then was diluted with ethyl acetate (50 ml). The dilution was washed with saturated brine, dried over anhydrous magnesium sulfate, and concentrated under reduced pressure. The resulting residue was purified by chromatography, and was recrystallized from a mixed solvent of ethyl acetate/hexane. Thus, the title compound (164 mg) was obtained as a light pink solid.

¹H NMR (400 MHz, DMSO-d₆) δ ppm 1.81 (2H, tt, J=7.1, 6.3 Hz), 3.06 (2H, t, J=7.1 Hz), 3.21 (3H, s), 3.36 (2H, s), 3.38-3.47 (6H, m), 4.86 (2H, q, J=8.8 Hz), 7.19 (2H, d, J=9.0 Hz), 7.31 (2H, d, J=9.0 Hz), 11.09 (1H, s).

### Example 300

### 3-[4-(Cyclopropylmethoxy)phenyl]-2-(ethylsulfanyl)-5,7-dihydro-3H-pyrrolo[2,3-d]pyrimidine-4,6-dione

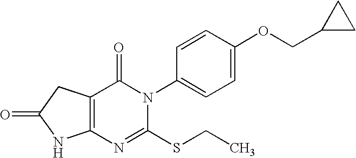

A mixture of 3-[4-(cyclopropylmethoxy)phenyl]-2-thioxo-2,3,5,7-tetrahydro-1H-pyrrolo[2,3-d]pyrimidine-4,6-dione (200 mg) obtained by the method of Example 48, or a method pursuant to thereto, a 1 M aqueous solution of sodium hydrogen carbonate (607 μl), iodoethane (245 μl) and acetonitrile (5.5 ml) was heated to reflux for 18 hours. The reaction mixture was returned to room temperature, and then was diluted with ethyl acetate (50 ml). The dilution was washed with saturated brine, dried over anhydrous magnesium sulfate, and then concentrated under reduced pressure. The resulting residue was purified by chromatography, and thus the title compound (176 mg) was obtained as a yellowish white solid.

¹H NMR (400 MHz, DMSO-d₆) δ ppm 0.32-0.38 (2H, m), 0.56-0.64 (2H, m), 1.16-1.31 (1H, m), 1.22 (4H, t, J=7.3 Hz), 3.01 (2H, q, J=7.3 Hz), 3.36 (2H, s), 3.87 (2H, d, J=7.1 Hz), 7.03 (2H, d, J=9.0 Hz), 7.19 (2H, d, J=9.0 Hz), 11.08 (1H, s).

### Example 301

### 3-[4-(Cyclopropylmethoxy)phenyl]-2-{[3-(methylsulfonyl)propyl]sulfanyl}-5,7-dihydro-3H-pyrrolo[2,3-d]pyrimidine-4,6-dione

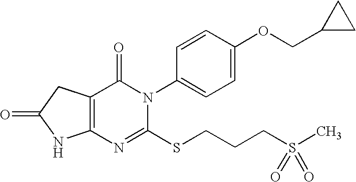

A mixture of 3-[4-(cyclopropylmethoxy)phenyl]-2-thioxo-2,3,5,7-tetrahydro-1H-pyrrolo[2,3-d]pyrimidine-4,6-dione (200 mg) obtained by the method of Example 48, or a method pursuant to thereto, a 1 M aqueous solution of sodium hydrogen carbonate (607 μl), 3-(methylsulfonyl)propyl 4-methylbenzenesulfonate (195 mg) obtained by the method described in a published document, WO 08/1931, or a method pursuant to thereto, and acetonitrile (5.5 ml) was heated to reflux for 18 hours. The reaction mixture was returned to room temperature, and then was diluted with ethyl acetate (50 ml). The dilution was washed with saturated brine, dried over anhydrous magnesium sulfate, and then concentrated under reduced pressure. The resulting residue was purified by chromatography, and thus the title compound (158 mg) was obtained as a white solid.

¹H NMR (400 MHz, DMSO-d₆) δ ppm 0.31-0.39 (2H, m), 0.56-0.64 (2H, m), 1.19-1.33 (1H, m), 1.97-2.12 (2H, m), 2.97 (3H, s), 3.08-3.21 (4H, m), 3.37(2H, s), 3.87 (2H, d, J=6.8 Hz), 7.05 (2H, d, J=9.0 Hz), 7.23 (2H, d, J=9.0 Hz), 11.10 (1H, s).

### Example 302

### 2-[(3-Ethoxypropyl)sulfanyl]-3-[4-(2,2,2-trifluoroethoxy)phenyl]-5,7-dihydro-3H-pyrrolo[2,3-d]pyrimidine-4,6-dione

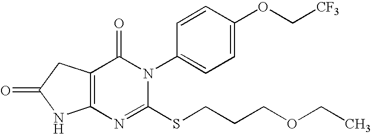

A mixture of 2-thioxo-3-[4-(2,2,2-trifluoroethoxy)phenyl]-2,3,5,7-tetrahydro-1H-pyrrolo[2,3-d]pyrimidine-4,6-dione (200 mg) obtained by the method of Example 46, or a method pursuant to thereto, a 1 M aqueous solution of sodium hydrogen carbonate (560 μl), 3-ethoxypropyl 4-methylbenzenesulfonate (174 mg) obtained by a method described in a published document, Canadian Journal of Chemistry (Can. J. Chem.), Vol. 33, p. 1207 (1955), or a method pursuant to thereto, and acetonitrile (5.5 ml) was heated to reflux for one hour. The reaction mixture was returned to room temperature, and then was diluted with ethyl acetate (50 ml). The dilution was washed with saturated brine, dried over anhydrous magnesium sulfate, and then concentrated under reduced pressure. The resulting residue was purified by chromatography, and was recrystallized from a mixed solvent of ethyl acetate/hexane. Thus, the title compound (254 mg) was obtained as white needle-shaped crystals.

¹H NMR (400 MHz, DMSO-d₆) δ ppm 1.06 (3H, t, J=7.1 Hz), 1.75-1.87 (2H, m), 3.06 (2H, t, J=7.2 Hz), 3.34-3.41 (6H, m), 4.86 (2H, q, J=9.0 Hz), 7.19 (2H, d, J=9.0 Hz), 7.31 (2H, d, J=9.0 Hz), 11.09 (1H, s).

### Example 303

### 2-(Methylsulfanyl)-3-[4-(2,2,3,3,3-pentafluoropropoxy)phenyl]-5,7-dihydro-3H-pyrrolo[2,3-d]pyrimidine-4,6-dione

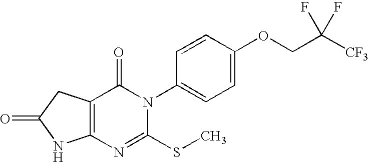

A mixture of 3-[4-(2,2,3,3,3-pentafluoropropoxy)phenyl]-2-thioxo-2,3,5,7-tetrahydro-1H-pyrrolo[2,3-d]pyrimidine-4,6-dione (4.16 g) obtained by the method of Example 47, or a method pursuant to thereto, iodomethane (5 ml), a 1 M aqueous solution of sodium hydrogen carbonate (10 ml) and acetonitrile (50 ml) was stirred for one hour at room temperature. Subsequently, water was added thereto, and then the mixture was extracted with ethyl acetate. The organic layer was washed with water and saturated brine, dried over anhydrous sodium sulfate, and then concentrated under reduced pressure. The residue was purified by chromatography and the solid suspended in an ethyl acetate/hexane mixed solvent was filtered to obtain a yellow solid (3.52 g). This solid (200 mg) was recrystallized from a mixed solvent of ethyl acetate/hexane, and thus the title compound (120 mg) was obtained as a white solid.

¹H NMR (300 MHz, DMSO-d₆) δ ppm 2.41 (3H, s), 3.36 (2H, s), 4.93 (2H, t, J=13.1 Hz), 7.21 (2H, d, J=8.9 Hz), 7.32 (2H, d, J=8.9 Hz), 11.09 (1H, s).

### Example 304

### 2-(Methylsulfinyl)-3-[4-(2,2,3,3,3-pentafluoropropoxy)phenyl]-5,7-dihydro-3H-pyrrolo[2,3-d]pyrimidine-4,6-dione

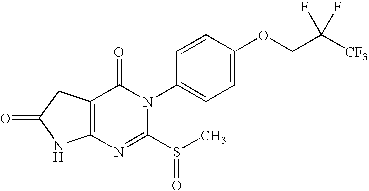

To a mixture of 2-(methylsulfanyl)-3-[4-(2,2,3,3,3-pentafluoropropoxy)phenyl]-5,7-dihydro-3H-pyrrolo[2,3-d]pyrimidine-4,6-dione (3.32 g) obtained by the method of Example 303, or a method pursuant to thereto, and methanol (200 ml) at 40° C., a solution of Oxone (registered trademark) monopersulfate compound (6.14 g) in water (20 ml) was added dropwise. The resulting mixture was stirred for one hour at 60° C., and then was concentrated under reduced pressure. Water was added to the residue and then a solid generated therefrom was collected by filtration, washed with water and diisopropyl ether, and then dried, to obtain a pale red solid (3.26 g). This solid (100 mg) was recrystallized from ethyl acetate, and thus the title compound (43 mg) was obtained as a white solid.

¹H NMR (300 MHz, DMSO-d₆) δ ppm 2.69 (3H, s), 3.49 (2H, s), 4.94 (2H, t, J=13.3 Hz), 7.17-7.28 (2H, m), 7.40-7.46 (1H, m), 7.52-7.58 (1H, m), 11.43 (1H, s).

### Example 305

### 2-Methoxy-3-[4-(2,2,3,3,3-pentafluoropropoxy)phenyl]-5,7-dihydro-3H-pyrrolo[2,3-d]pyrimidine-4,6-dione

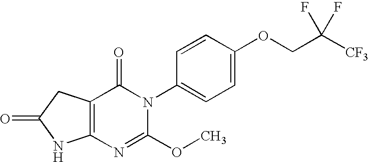

To a mixture of 2-(methylsulfinyl)-3-[4-(2,2,3,3,3-pentafluoropropoxy)phenyl]-5,7-dihydro-3H-pyrrolo[2,3-d]pyrimidine-4,6-dione (250 mg) obtained by the method of Example 304, or a method pursuant to thereto, methanol (20 ml) and tetrahydrofuran (10 ml) in an ice water bath, a methanol (1 ml) solution of a 28% sodium methoxide-methanol solution (120 μl) was added dropwise. The mixture was stirred for 30 minutes in an ice water bath, and water and ethyl acetate were added thereto. The pH of the mixture was adjusted to about 4 with a 5% aqueous solution of citric acid, and then the mixture was extracted with ethyl acetate. The organic layer was washed with water and saturated brine, dried over anhydrous sodium sulfate, and then concentrated under reduced pressure. The residue was purified by chromatography, and then was recrystallized from a mixed solvent of ethyl acetate/hexane. Thus, the title compound (63 mg) was obtained as a white solid.

¹H NMR (300 MHz, DMSO-d₆) δ ppm 3.33 (2H, s), 3.84 (3H, s), 4.89 (2H, t, J=13.3 Hz), 7.15 (2H, d, J=8.7 Hz), 7.25 (2H, d, J=8.7 Hz), 11.07 (1H, s).

### Example 306

### 3-[4-(2,2,3,3,3-Pentafluoropropoxy)phenyl]-2-(2,2,2-trifluoroethoxy)-5,7-dihydro-3H-pyrrolo[2,3-d]pyrimidine-4,6-dione

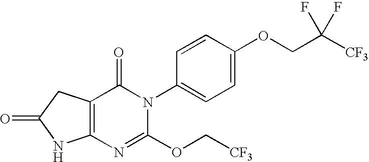

A mixture of 2,2,2-trifluoroethanol (1 ml), sodium hydride (60% in oil, 24 mg) and tetrahydrofuran (3 ml) was added to a mixture of 2-(methylsulfinyl)-3-[4-(2,2,3,3,3-pentafluoropropoxy)phenyl]-5,7-dihydro-3H-pyrrolo[2,3-d]pyrimidine-4,6-dione (250 mg) obtained by the method of Example 304, or a method pursuant to thereto, and tetrahydrofuran (10 ml) in an ice water bath. The mixture was stirred for 30 minutes in an ice water bath, and water and ethyl acetate were added thereto. The pH of the mixture was adjusted to about 4 with a 5% aqueous solution of citric acid, and then the mixture was extracted with ethyl acetate. The organic layer was washed with water and saturated brine, dried over anhydrous sodium sulfate, and then concentrated under reduced pressure. The residue was purified by chromatography, and then was recrystallized from a mixed solvent of ethyl acetate/hexane. Thus, the title compound (93 mg) was obtained as a pale red solid.

¹H NMR (300 MHz, DMSO-d₆) δ ppm 3.37 (2H, s), 4.83-5.05 (4H, m), 7.17 (2H, d, J=9.0 Hz), 7.27 (2H, d, J=9.0 Hz), 11.17(1H, s).

### Example 307

### 2-(Ethyl sulfanyl)-3-[3-fluoro-4-(2,2,2-trifluoroethoxy)phenyl]-5,7-dihydro-3H-pyrrolo[2,3-d]pyrimidine-4,6-dione

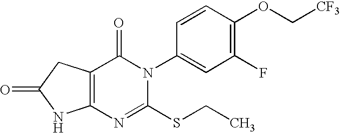

A 1 M aqueous solution of sodium hydrogen carbonate (4.76 ml) was added to a mixture of 3-[3-fluoro-4-(2,2,2-trifluoroethoxy)phenyl]-2-thioxo-2,3,5,7-tetrahydro-1H-pyrrolo[2,3-d]pyrimidine-4,6-dione (1.70 g) obtained by the method of Example 49, or a method pursuant to thereto, iodoethane (0.57 ml) and N,N-dimethylformamide (20 ml), and the resulting mixture was stirred for one hour at 70° C. The reaction mixture was returned to room temperature, and then the solvent was distilled off under reduced pressure. The resulting residue was diluted with ethyl acetate, and the dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (1.05 g) was obtained as a white powder.

¹H NMR (300 MHz, DMSO-d₆) δ ppm 1.24 (3H, t, Hz), 3.05 (2H, q, J=7.3 Hz), 3.31 (2H, s), 4.96 (2H, q, J=8.7 Hz), 7.18-7.25 (1H, m), 7.38-7.51 (2H, m), 11.11 (1H, s).

### Example 308

### 3-[3-Fluoro-4-(2,2,2-trifluoroethoxy)phenyl]-2-(methylsulfanyl)-5,7-dihydro-3H-pyrrolo[2,3-d]pyrimidine-4,6-dione

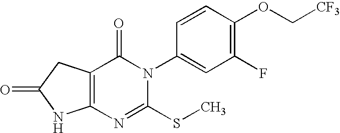

A 1 M aqueous solution of sodium hydrogen carbonate (0.47 ml) was added to a mixture of 3-[3-fluoro-4-(2,2,2-trifluoroethoxy)phenyl]-2-thioxo-2,3,5,7-tetrahydro-1H-pyrrolo[2,3-d]pyrimidine-4,6-dione (170 mg) obtained by the method of Example 49, or a method pursuant to thereto, iodomethane (333 mg) and N,N-dimethylformamide (6 ml), and the resulting mixture was stirred for one hour at 50° C. The reaction mixture was returned to room temperature, and then the solvent was distilled off under reduced pressure. The resulting residue was diluted with ethyl acetate, and the dilution was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (143 mg) was obtained as a white powder.

¹H NMR (300 MHz, DMSO-d₆) δ ppm 2.43 (3H, s), 3.37 (2H, s), 4.97 (2H, q, J=8.7 Hz), 7.20-7.26 (1H, m), 7.40-7.45 (1H, m), 7.45-7.52(1H, m), 11.13 (1H, s).

### Example 309

### 3-[3-Fluoro-4-(2,2,2-trifluoroethoxy)phenyl]-2-(2,2,2-trifluoroethoxy)-5,7-dihydro-3H-pyrrolo[2,3-d]pyrimidine-4,6-dione

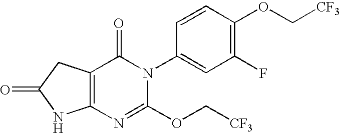

To a methanol (20 ml) solution of 2-(ethylsulfanyl)-3-[3-fluoro-4-(2,2,2-trifluoroethoxy)phenyl]-5,7-dihydro-3H-pyrrolo[2,3-d]pyrimidine-4,6-dione (650 mg) obtained by the method of Example 307, or a method pursuant to thereto, an aqueous solution of Oxone (registered trademark) monopersulfate compound (991 mg) (20 ml) was added at room temperature, and then the mixture was stirred for 30 minutes at 80° C. The reaction mixture was returned to room temperature, and then methanol was distilled off under reduced pressure. The aqueous solution obtained therefrom was extracted with ethyl acetate. The organic layer obtained therefrom was washed with saturated brine, dried over anhydrous magnesium sulfate, and then concentrated under reduced pressure, to obtain a brown powder (550 mg). This powder was dissolved in N,N-dimethylformamide (10 ml), and the resulting solution was added dropwise to a mixture of sodium hydride (60% in oil, 57.2 mg), 2,2,2-trifluoroethanol (20 ml) and tetrahydrofuran (10 ml) under ice cooling. The mixture was stirred for 10 minutes at room temperature. The reaction mixture was poured into 0.5 M hydrochloric acid, and then the resultant was extracted with ethyl acetate. The organic layer was washed with water and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (135 mg) was obtained as a white powder.

¹H NMR (300 MHz, DMSO-d₆) δ ppm 3.38 (2H, s), 4.87-5.04 (4H, m), 7.13-7.21 (1H, m), 7.35-7.45(2H, m), 11.18(1H, s).

### Example 310

### 2-(1-Methylethoxy)-3-[4-(2,2,3,3,3-pentafluoropropoxy)phenyl]-5,7-dihydro-3H-pyrrolo[2,3-d]pyrimidine-4,6-dione

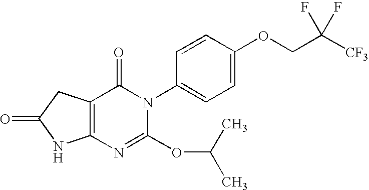

A mixture of propan-2-ol (1 ml), sodium hydride (60% in oil, 28 mg) and tetrahydrofuran (3 ml) was added to a mixture of 2-(methylsulfinyl)-3-[4-(2,2,3,3,3-pentafluoropropoxy)phenyl]-5,7-dihydro-3H-pyrrolo[2,3-d]pyrimidine-4,6-dione (300 mg) obtained by the method of Example 304, or a method pursuant to thereto, and propan-2-ol (20 ml) in an ice water bath. The mixture was stirred for 30 minutes in an ice water bath, and water and ethyl acetate were added thereto. The pH of the mixture was adjusted to about 4 with a 5% aqueous solution of citric acid, and then the mixture was extracted with ethyl acetate. The organic layer was washed with water and saturated brine, dried over anhydrous sodium sulfate, and then concentrated under reduced pressure. The residue was purified by chromatography and reverse phase chromatography, and then was recrystallized from a mixed solvent of ethyl acetate/hexane. Thus, the title compound (31 mg) was obtained as a pale red solid.

¹H NMR (300 MHz, DMSO-d₆) δ ppm 1.17 (6H, d, J=6.2 Hz), 3.32 (2H, s), 4.90 (2H, t, J=13.3 Hz), 5.19 (1H, spt, J=6.2 Hz), 7.14 (2H, d, J=9.0 Hz), 7.22 (2H, d, J=9.0 Hz), 11.02 (1H, s).

### Example 311

### 2-(4-Fluorophenoxy)-3-[4-(2,2,3,3,3-pentafluoropropoxy)phenyl]-5,7-dihydro-3H-pyrrolo[2,3-d]pyrimidine-4,6-dione

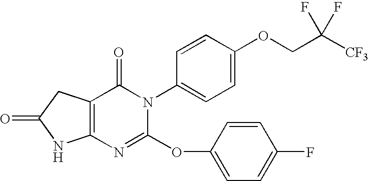

A mixture of 4-fluorophenol (112 mg), sodium hydride (60% in oil, 28 mg) and tetrahydrofuran (10 ml) was added to a mixture of 2-(methylsulfinyl)-3-[4-(2,2,3,3,3-pentafluoropropoxy)phenyl]-5,7-dihydro-3H-pyrrolo[2,3-d]pyrimidine-4,6-dione (300 mg) obtained by the method of Example 304, or a method pursuant to thereto, and tetrahydrofuran (20 ml) in an ice water bath. The mixture was stirred for 30 minutes in an ice water bath and for 4 hours at room temperature, and water and ethyl acetate were added thereto. The pH of the mixture was adjusted to about 4 with a 5% aqueous solution of citric acid, and then the mixture was extracted with ethyl acetate. The organic layer was washed with water and saturated brine, dried over anhydrous sodium sulfate, and then concentrated under reduced pressure. The residue was purified by chromatography, and then was recrystallized from a mixed solvent of ethyl acetate/hexane. Thus, the title compound (52 mg) was obtained as a white solid.

¹H NMR (300 MHz, DMSO-d₆) δ ppm 3.35 (2H, s), 4.90 (2H, t, J=13.3 Hz), 7.20 (2H, d, J=8.9 Hz), 7.22-7.35 (4H, m), 7.48 (2H, d, J=8.9 Hz), 11.02 (1H, s).

### Example 312

### ({4,6-Dioxo-3-[4-(2,2,2-trifluoroethoxy)phenyl]-4,5,6,7-tetrahydro-3H-pyrrolo[2,3-d]pyrimidin-2-yl}sulfanyl)acetonitrile

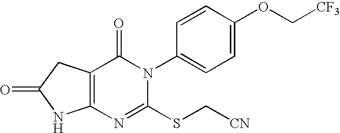

A mixture of 2-thioxo-3-[4-(2,2,2-trifluoroethoxy)phenyl]-2,3,5,7-tetrahydro-1H-pyrrolo[2,3-d]pyrimidine-4,6-dione (180 mg) obtained by the method of Example 46, or a method pursuant to thereto, a 1 M aqueous solution of sodium hydrogen carbonate (504 μl), bromoacetonitrile (224 μl) and acetonitrile (5 ml) was heated to reflux for 30 minutes. The reaction mixture was returned to room temperature, and then was diluted with ethyl acetate (50 ml). The dilution was washed with saturated brine, dried over anhydrous magnesium sulfate, and then concentrated under reduced pressure. The resulting residue was purified by chromatography, and thus the title compound (194 mg) was obtained as a yellowish white solid.

¹H NMR (300 MHz, DMSO-d₆) δ ppm 3.41 (2H, s), 4.10 (2H, s), 4.87 (2H, q, J=8.8 Hz), 7.23 (2H, d, J=9.1 Hz), 7.39 (2H, d, J=9.1 Hz), 11.23 (1H, s).

### Example 313

### 2-[(2-Methylpropyl)sulfanyl]-3-[4-(2,2,2-trifluoroethoxy)phenyl]-5,7-dihydro-3H-pyrrolo[2,3-d]pyrimidine-4,6-dione

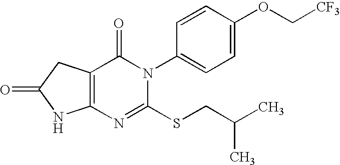

A mixture of 2-thioxo-3-[4-(2,2,2-trifluoroethoxy)phenyl]-2,3,5,7-tetrahydro-1H-pyrrolo[2,3-d]pyrimidine-4,6-dione (180 mg) obtained by the method of Example 46, or a method pursuant to thereto, a 1 M aqueous solution of sodium hydrogen carbonate (504 μl), 1-iodo-2-methylpropane (292 μl) and acetonitrile (5 ml) was heated to reflux for one hour. The reaction mixture was returned to room temperature, and then was diluted with ethyl acetate (50 ml). The dilution was washed with saturated brine, dried over anhydrous magnesium sulfate, and then concentrated under reduced pressure. The resulting residue was purified by chromatography, and was recrystallized from a mixed solvent of ethyl acetate/hexane. Thus, the title compound (131 mg) was obtained as light pink needle-shaped crystals.

¹H NMR (300 MHz, DMSO-d₆) δ ppm 0.91 (6H, d, J=6.4 Hz), 1.76-1.95 (1H, m), 2.97 (2H, d, J=6.4 Hz), 3.36 (2H, s), 4.86 (2H, q, J=8.9 Hz), 7.19 (2H, d, J=9.1 Hz), 7.30 (2H, d, J=9.1 Hz), 11.04 (1H, s).

### Example 314

### 2-({4,6-Dioxo-3-[4-(2,2,2-trifluoroethoxy)phenyl]-4,5,6,7-tetrahydro-3H-pyrrolo[2,3-d]pyrimidin-2-yl}sulfanyl)-N,N-diethyl acetamide

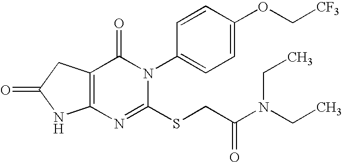

A mixture of 2-thioxo-3-[4-(2,2,2-trifluoroethoxy)phenyl]-2,3,5,7-tetrahydro-1H-pyrrolo[2,3-d]pyrimidine-4,6-dione (180 mg) obtained by the method of Example 46, or a method pursuant to thereto, a 1 M aqueous solution of sodium hydrogen carbonate (504 μl), N,N-diethylchloroacetamide (83 μl) and acetonitrile (5 ml) was heated to reflux for one hour. The reaction mixture was returned to room temperature, and then was diluted with ethyl acetate (50 ml). The dilution was washed with saturated brine, dried over anhydrous magnesium sulfate, and then concentrated under reduced pressure. The resulting residue was purified by chromatography, and thus the title compound (218 mg) was obtained as a white solid.

¹H NMR (300 MHz, DMSO-d₆) δ ppm 0.99 (3H, t, J=7.1 Hz), 1.15 (3H, t, J=7.3 Hz), 3.25 (2H, q, J=7.1 Hz), 3.33-3.43 (4H, m), 4.11 (2H, s), 4.88 (2H, q, J=8.9 Hz), 7.22 (2H, d, J=9.0 Hz), 7.31 (2H, d, J=9.0 Hz), 11.04 (1H, s).

### Example 315

### 2-[(2-Hydroxy-2-methylpropyl)sulfanyl]-3-[4-(2,2,2-trifluoroethoxy)phenyl]-5,7-dihydro-3H-pyrrolo[2,3-d]pyrimidine-4,6-dione

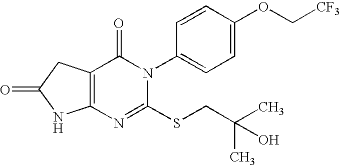

A mixture of 2-thioxo-3-[4-(2,2,2-trifluoroethoxy)phenyl]-2,3,5,7-tetrahydro-1H-pyrrolo[2,3-d]pyrimidine-4,6-dione (300 mg) obtained by the method of Example 46, or a method pursuant to thereto, a 1 M aqueous solution of sodium hydrogen carbonate (840 μl), isobutylene oxide (749 μl) and acetonitrile (8.5 ml) was heated to 60° C., and the resulting mixture was stirred for one hour. The reaction mixture was returned to room temperature, and then 1 M hydrochloric acid (3 ml) and ethyl acetate (80 ml) were added thereto. The aqueous layer was neutralized with a saturated aqueous solution of sodium hydrogen carbonate. The layers were separated, and the organic layer was washed with saturated brine, dried over anhydrous magnesium sulfate, and then concentrated under reduced pressure. The resulting residue was purified by chromatography, and was washed with diethyl ether, and thus the title compound (275 mg) was obtained as a white solid.

¹H NMR (300 MHz, DMSO-d₆) δ ppm 1.14 (6H, s), 3.22 (2H, s), 3.35 (2H, s), 4.74 (1H, s), 4.87 (2H, q, J=8.9 Hz), 7.20 (2H, d, J=9.0 Hz), 7.31 (2H, d, J=9.0 Hz), 11.03 (1H, s).

### Example 316

### 3-[3-Fluoro-4-(2,2,2-trifluoroethoxy)phenyl]-2-(1-methylethoxy)-3,7-dihydro-4H-pyrrolo[2,3-d]pyrimidin-4-one

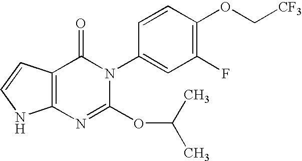

To a solution of propan-2-ol (3.58 g) in N,N-dimethylformamide (50 ml), sodium hydride (60% in oil, 1.93 g) was added. The resulting mixture was stirred for 30 minutes at room temperature. To this mixture, a N,N-dimethylformamide (20 ml) solution of 2-(ethylsulfinyl)-3-[3-fluoro-4-(2,2,2-trifluoroethoxy)phenyl]-3,7-dihydro-4H-pyrrolo[2,3-d]pyrimidin-4-one (3.90 g) obtained by the method of Example 263, or a method pursuant to thereto was added. The resulting mixture was stirred for one hour at room temperature. The solvent was distilled off under reduced pressure, and then the resulting residue was diluted with ethyl acetate. The dilution was washed with 0.1 M hydrochloric acid and saturated brine, dried over anhydrous magnesium sulfate, and then concentrated under reduced pressure. The resulting residue was purified by chromatography, and thus the title compound (3.51 g) was obtained as a pale pink powder.

¹H NMR (300 MHz, DMSO-d₆) δ ppm 1.18 (6H, d, J=6.1 Hz), 4.93 (2H, q, J=8.7 Hz), 5.17 (1H, spt, J=6.1 Hz), 6.35-6.40 (1H, m), 6.83-6.91 (1H, m), 7.07-7.13 (1H, m), 7.28-7.41 (2H, m), 11.68 (1H, br. s.).

### Example 317

### 3-[3-Fluoro-4-(2,2,2-trifluoroethoxy)phenyl]-2-(1-methylethoxy)-5,7-dihydro-3H-pyrrolo[2,3-d]pyrimidine-4,6-dione

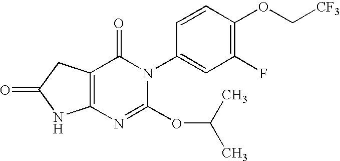

A mixture of 3-[3-fluoro-4-(2,2,2-trifluoroethoxy)phenyl]-2-(1-methylethoxy)-3,7-dihydro-4H-pyrrolo[2,3-d]pyrimidin-4-one (3.4 g) obtained by the method of Example 316, or a method pursuant to thereto, iodosobenzene diacetate (2.84 g) and acetic acid (30 ml) was stirred for 2 hours at 100° C. The reaction mixture was returned to room temperature, and then the solvent was distilled off under reduced pressure. The resulting residue was purified by chromatography, and then was recrystallized from a mixed solvent of ethyl acetate/hexane. Thus, the title compound (138 mg) was obtained as a white powder.

¹H NMR (300 MHz, DMSO-d₆) δ ppm 1.18 (6H, d, J=6.2 Hz), 3.32 (2H, s), 4.93 (2H, q, J=8.8 Hz), 5.13-5.24 (1H, m), 7.08-7.14 (1H, m), 7.32-7.40(2H, m), 11.05 (1H, s).

### Example 318

### 2-(2-Methylpropoxy)-3-[4-(2,2,2-trifluoroethoxy)phenyl]-5,7-dihydro-3H-pyrrolo[2,3-d]pyrimidine-4,6-dione

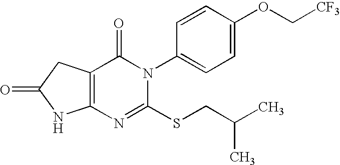

Sodium hydride (60% in oil, 47 mg) was added to 2-methylpropan-1-ol (10 ml) under ice cooling, and the resulting mixture was stirred for 10 minutes at room temperature. To this mixture, 2-(methylsulfinyl)-3-[4-(2,2,2-trifluoroethoxy)phenyl]-5,7-dihydro-3H-pyrrolo[2,3-d]pyrimidine-4,6-dione (350 mg) was added. The resulting mixture was stirred for 30 minutes at room temperature. The solvent was distilled off under reduced pressure, and then the resulting residue was diluted with ethyl acetate. The dilution was washed with 1 M hydrochloric acid and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (30 mg) was obtained as a white powder.

¹H NMR (300 MHz, DMSO-d₆) δ ppm 0.72 (6H, d, J=6.6 Hz), 1.74-1.88 (1H, m), 3.32 (2H, s), 4.03 (2H, d, J=6.0 Hz), 4.83 (2H, q, J=8.9 Hz), 7.14 (2H, d, J=9.0 Hz), 7.25 (2H, d, J=9.0 Hz), 11.03 (1H, s).

### Example 319

### 2-(2-Cyclopropylethoxy)-3-[4-(2,2,2-trifluoroethoxy)phenyl]-5,7-dihydro-3H-pyrrolo[2,3-d]pyrimidine-4,6-dione

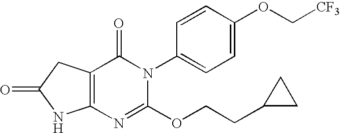

Sodium hydride (60% in oil, 47 mg) was added to 2-cyclopropylethanol (10 ml) under ice cooling, and the resulting mixture was stirred for 10 minutes at room temperature. To this mixture, 2-(methylsulfinyl)-3-[4-(2,2,2-trifluoroethoxy)phenyl]-5,7-dihydro-3H-pyrrolo[2,3-d]pyrimidine-4,6-dione (350 mg) was added. The resulting mixture was stirred for 30 minutes at room temperature. The solvent was distilled off under reduced pressure, and then the resulting residue was diluted with ethyl acetate. The dilution was washed with 1 M hydrochloric acid and saturated brine, dried over anhydrous magnesium sulfate, and then concentrated under reduced pressure. The resulting residue was purified by chromatography, and then was recrystallized from a mixed solvent of ethyl acetate/hexane. Thus, the title compound (56 mg) was obtained as a white powder.

¹H NMR (300 MHz, DMSO-d₆) δ ppm −0.13-−0.05 (2H, m), 0.23-0.33 (2H, m), 0.44-0.59 (1H, m), 1.35-1.47 (2H, m), 3.32 (2H, s), 4.31 (2H, t, J=6.4 Hz), 4.82 (2H, q, J=8.9 Hz), 7.13 (2H, d, J=9.0 Hz), 7.23 (2H, d, J=9.0 Hz), 11.04 (1H, br. s.).

### Example 320

### 2-(Tetrahydro-2H-pyran-4-yloxy)-3-[4-(2,2,2-trifluoroethoxy)phenyl]-3,7-dihydro-4H-pyrrolo[2,3-d]pyrimidin-4-one

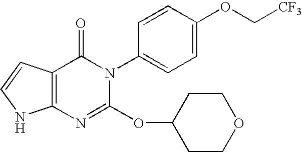

To a N,N-dimethylformamide (15 ml) solution of tetrahydro-2H-pyran-4-ol (3.0 ml), sodium hydride (60% in oil, 392 mg) was added. The resulting mixture was stirred for 30 minutes at room temperature. To this mixture, a N,N-dimethylformamide (20 ml) solution of 2-(ethylsulfinyl)-3-[4-(2,2,2-trifluoroethoxy)phenyl]-3,7-dihydro-4H-pyrrolo[2,3-d]pyrimidin-4-one (2.8 g) obtained by the method of Example 206, or a method pursuant to thereto was added. The resulting mixture was stirred for 15 hours at 80° C. The reaction mixture was returned to room temperature, and then the solvent was distilled off under reduced pressure. The resulting residue was diluted with ethyl acetate, and the dilution was washed with 1 M hydrochloric acid and saturated brine, dried over anhydrous magnesium sulfate, and then concentrated under reduced pressure. The resulting residue was purified by chromatography, and thus the title compound (1.05 g) was obtained as a white powder.

¹H NMR (300 MHz, DMSO-d₆) δ ppm 1.44-1.58 (2H, m), 1.80-1.94 (2H, m), 3.34-3.51 (4H, m), 4.84 (2H, q, J=9.0 Hz), 5.13-5.23 (1H, m), 6.38 (1H, d, J=3.4 Hz), 6.88 (1H, d, J=3.4 Hz), 7.16 (2H, d, J=9.0 Hz), 7.26 (2H, d, J=9.0 Hz), 11.68 (1H, br. s.).

### Example 321

### 2-(Tetrahydro-2H-pyran-4-yloxy)-3-[4-(2,2,2-trifluoroethoxy)phenyl]-5,7-dihydro-3H-pyrrolo[2,3-d]pyrimidine-4,6-dione

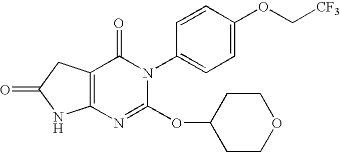

To a mixture of 2-(tetrahydro-2H-pyran-4-yloxy)-3-[4-(2,2,2-trifluoroethoxy)phenyl]-3,7-dihydro-4H-pyrrolo[2,3-d]pyrimidin-4-one (650 mg) obtained by the method of Example 320, or a method pursuant to thereto, 2-methylpropan-2-ol (20 ml) and water (5 ml), a 2-methylpropan-2-ol solution of bromine (2.22 M, 0.71 ml), which had been prepared previously, was added dropwise under ice cooling. The mixture was stirred for 30 minutes under ice cooling. A 10% aqueous solution of sodium thiosulfate was added to the reaction mixture. The mixture was stirred for 10 minutes, and was extracted with ethyl acetate. The organic layer was washed with saturated brine, dried over anhydrous magnesium sulfate, and then concentrated under reduced pressure. The resulting residue was purified by chromatography, and thus the title compound (21 mg) was obtained as a white powder.

¹H NMR (300 MHz, DMSO-d₆) δ ppm 1.43-1.59 (2H, m), 1.77-1.95 (2H, m), 3.33(2H, s), 3.34-3.49 (4H, m), 4.84 (2H, q, J=8.9 Hz), 5.15-5.25 (1H, m), 7.16(2H, d, J=9.0 Hz), 7.27 (2H, d, J=9.0 Hz), 11.03 (1H, br. s.).

### Example 322

### 2-[(Cyclopropylmethyl)sulfanyl]-3-[3-fluoro-4-(2,2,2-trifluoroethoxy)phenyl]-5,7-dihydro-3H-pyrrolo[2,3-d]pyrimidine-4,6-dione

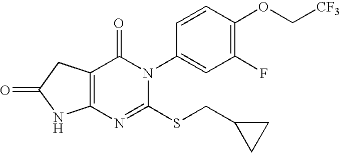

A mixture of 3-[3-fluoro-4-(2,2,2-trifluoroethoxy)phenyl]-2-thioxo-2,3,5,7-tetrahydro-1H-pyrrolo[2,3-d]pyrimidine-4,6-dione (300 mg) obtained by the method of Example 49, or a method pursuant to thereto, a 1 M aqueous solution of sodium hydrogen carbonate (800 μl), (bromomethyl)cyclopropane (391 μl) and acetonitrile (5 ml) was heated to reflux for 40 minutes. The reaction mixture was returned to room temperature, and then was diluted with ethyl acetate (80 ml). The dilution was washed with water and saturated brine, dried over anhydrous magnesium sulfate, and then concentrated under reduced pressure. The resulting residue was purified by chromatography, and thus the title compound (265 mg) was obtained as a white solid.

¹H NMR (300 MHz, DMSO-d₆) δ ppm 0.21-0.28 (2H, m), 0.47-0.56 (2H, m), 1.03-1.12 (1H, m), 2.99 (1H, dd, J=13.3, 7.2 Hz), 3.04 (1H, dd, J=13.3, 7.2 Hz), 3.36 (2H, s), 4.97 (2H, q, J=8.8 Hz), 7.22 (1H, ddd, J=8.7, 2.3, 1.5 Hz), 7.44 (1H, dd, J=9.1, 8.7 Hz), 7.48 (1H, dd, J=11.7, 2.3 Hz), 11.08 (1H, br. s.).

### Example 323

### 2-[(Cyclopropylmethyl)sulfanyl]-3-[4-(2,2,3,3,3-pentafluoropropoxy)phenyl]-5,7-dihydro-3H-pyrrolo[2,3-d]pyrimidine-4,6-dione

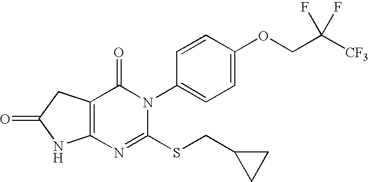

A mixture of 3-[4-(2,2,3,3,3-pentafluoropropoxy)phenyl]-2-thioxo-2,3,5,7-tetrahydro-1H-pyrrolo[2,3-d]pyrimidine-4,6-dione (500 mg) obtained by the method of Example 47, or a method pursuant to thereto, (bromomethyl)cyclopropane (500 μl), a 1 M aqueous solution of sodium hydrogen carbonate (1.2 ml) and acetonitrile (20 ml) was stirred for 3 hours at room temperature. To the mixture, water, ethyl acetate and a 5% aqueous solution of citric acid were added, and the mixture was extracted with ethyl acetate. The organic layer was washed with water and saturated brine, dried over anhydrous sodium sulfate, and then concentrated under reduced pressure. The residue was purified by chromatography, and then was recrystallized from a mixed solvent of ethyl acetate/hexane. Thus, the title compound (431 mg) was obtained as a white solid.

¹H NMR (300 MHz, DMSO-d₆) δ ppm 0.20-0.28 (2H, m), 0.47-0.55 (2H, m), 0.98-1.14 (1H, m), 3.00 (2H, d, J=7.3 Hz), 3.36 (2H, s), 4.94 (2H, t, J=13.3 Hz), 7.21 (2H, d, J=8.9 Hz), 7.31 (2H, d, J=8.9 Hz), 11.07 (1H, s).

### Example 324

### 2-[(1-Methylethyl)sulfanyl]-3-[4-(2,2,3,3,3-pentafluoropropoxy)phenyl]-5,7-dihydro-3H-pyrrolo[2,3-d]pyrimidine-4,6-dione

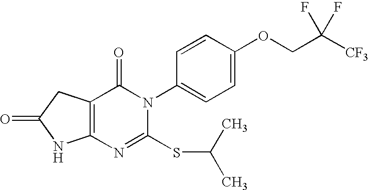

A mixture of 3-[4-(2,2,3,3,3-pentafluoropropoxy)phenyl]-2-thioxo-2,3,5,7-tetrahydro-1H-pyrrolo[2,3-d]pyrimidine-4,6-dione (500 mg) obtained by the method of Example 47, or a method pursuant to thereto, 2-iodopropane (500 μl), a 1 M aqueous solution of sodium hydrogen carbonate (1.2 ml) and acetonitrile (20 ml) was stirred for 3 hours at room temperature. To the mixture, water, ethyl acetate and a 5% aqueous solution of citric acid were added, and the mixture was extracted with ethyl acetate. The organic layer was washed with water and saturated brine, dried over anhydrous sodium sulfate, and then concentrated under reduced pressure. The residue was purified by chromatography, and then was recrystallized from a mixed solvent of ethyl acetate/hexane. Thus, the title compound (403 mg) was obtained as a white solid.

¹H NMR (300 MHz, DMSO-d₆) δ ppm 1.29 (6H, d, J=6.8 Hz), 3.36 (2H, s), 3.80 (1H, spt, J=6.8 Hz), 4.93 (2H, t, J=13.1 Hz), 7.19 (2H, d, J=9.0 Hz), 7.28 (2H, d, J=9.0 Hz), 11.07 (1H, s).

### Example 325

### 2-[(2-Methoxyethyl)sulfanyl]-3-[4-(2,2,2-trifluoroethoxy)phenyl]-5,7-dihydro-3H-pyrrolo[2,3-d]pyrimidine-4,6-dione

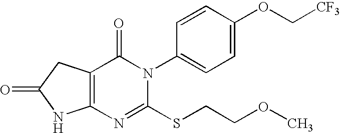

A mixture of 3-[4-(2,2,2-trifluoroethoxy)phenyl]-2-thioxo-2,3,5,7-tetrahydro-1H-pyrrolo[2,3-d]pyrimidine-4,6-dione (500 mg) obtained by the method of Example 46, or a method pursuant to thereto, 1-bromo-2-methoxyethane (500 μl), a 1 M aqueous solution of sodium hydrogen carbonate (1.5 ml) and acetonitrile (20 ml) was stirred for 3 hours at room temperature. To the mixture, water, ethyl acetate and a 5% aqueous solution of citric acid were added, and the mixture was extracted with ethyl acetate. The organic layer was washed with water and saturated brine, dried over anhydrous sodium sulfate, and then concentrated under reduced pressure. The residue was purified by chromatography, and then was recrystallized from ethyl acetate. Thus, the title compound (271 mg) was obtained as a pale red solid.

¹H NMR (300 MHz, DMSO-d₆) δ ppm 3.19-3.28 (5H, m), 3.37 (2H, s), 3.52 (2H, t, J=6.2 Hz), 4.86 (2H, q, J=8.9 Hz), 7.19 (2H, d, J=9.0 Hz), 7.30 (2H, d, J=9.0 Hz), 11.09 (1H, s).

### Example 326

### 2-(2,2,3,3,3-Pentafluoropropoxy)-3-[4-(2,2,2-trifluoroethoxy)phenyl]-5,7-dihydro-3H-pyrrolo[2,3-d]pyrimidine-4,6-dione

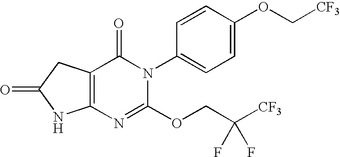

A mixture of 2,2,3,3,3-pentafluoropropan-1-ol (2 ml), sodium hydride (60% in oil, 220 mg) and tetrahydrofuran (5 ml) was added to a mixture of 2-(methylsulfinyl)-3-[4-(2,2,2-trifluoroethoxy)phenyl]-5,7-dihydro-3H-pyrrolo[2,3-d]pyrimidine-4,6-dione (1500 mg), 2,2,3,3,3-pentafluoropropan-1-ol (5 ml) and tetrahydrofuran (50 ml) in an ice water bath. The mixture was stirred for 1.5 hours in an ice water bath, diluted with water, ethyl acetate and a 5% aqueous solution of citric acid, and extracted with ethyl acetate. The organic layer was washed with water and saturated brine, dried over anhydrous sodium sulfate, and then concentrated under reduced pressure. The residue was purified by chromatography, and then was recrystallized from a mixed solvent of ethyl acetate/hexane. Thus, the title compound (815 mg) was obtained as a pale orange-colored solid.

¹H NMR (300 MHz, DMSO-d₆) δ ppm 3.38 (2H, s), 4.83 (2H, q, J=8.8 Hz), 5.04 (2H, t, J=12.9 Hz), 7.15 (2H, d, J=9.0 Hz), 7.24 (2H, d, J=9.0 Hz), 11.17 (1H, s).

### Example 327

### 2-({4,6-Dioxo-3-[4-(2,2,2-trifluoroethoxy)phenyl]-4,5,6,7-tetrahydro-3H-pyrrolo[2,3-d]pyrimidin-2-yl}sulfanyl)-N-ethyl acetamide

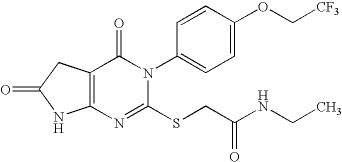

A mixture of 2-thioxo-3-[4-(2,2,2-trifluoroethoxy)phenyl]-2,3,5,7-tetrahydro-1H-pyrrolo[2,3-d]pyrimidine-4,6-dione (300 mg) obtained by the method of Example 46, or a method pursuant to thereto, a 1 M aqueous solution of sodium hydrogen carbonate (840 μl), 2-chloro-N-ethyl acetamide (102 mg) and acetonitrile (8.5 ml) was heated to 60° C., and the resulting mixture was stirred for one hour. The reaction mixture was returned to room temperature, and then was diluted with ethyl acetate (80 ml). The dilution was washed with water and saturated brine, dried over anhydrous magnesium sulfate, and then concentrated under reduced pressure. The resulting residue was purified by chromatography, and thus the title compound (318 mg) was obtained as a white solid.

¹H NMR (300 MHz, DMSO-d₆) δ ppm 0.99 (3H, t, J=7.3 Hz), 3.05 (2H, qd, J=7.3, 5.4 Hz), 3.37 (2H, s), 3.76 (2H, s), 4.87 (2H, q, J=8.9 Hz), 7.21 (2H, d, J=9.0 Hz), 7.33 (2H, d, J=9.0 Hz), 8.06 (1H, t, J=5.4 Hz), 11.06 (1H, s).

### Example 328

### N-(2-cyanoethyl)-2-({4,6-dioxo-3-[4-(2,2,2-trifluoroethoxy)phenyl]-4,5,6,7-tetrahydro-3H-pyrrolo[2,3-d]pyrimidin-2-yl}sulfanyl)acetamide

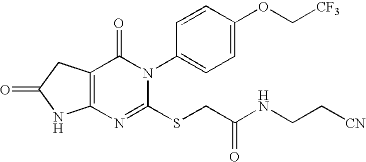

A mixture of 2-thioxo-3-[4-(2,2,2-trifluoroethoxy)phenyl]-2,3,5,7-tetrahydro-1H-pyrrolo[2,3-d]pyrimidine-4,6-dione (300 mg) obtained by the method of Example 46, or a method pursuant to thereto, a 1 M aqueous solution of sodium hydrogen carbonate (840 μl), 2-chloro-N-(2-cyanoethyl)acetamide (123 mg) obtained by the method of Reference Example 40, or a method pursuant to thereto, and acetonitrile (8.5 ml) was heated to 60° C., and the resulting mixture was stirred for one hour. The reaction mixture was returned to room temperature, and then was diluted with ethyl acetate (80 ml). The dilution was washed with water and saturated brine, dried over anhydrous magnesium sulfate, and then concentrated under reduced pressure. The resulting residue was purified by chromatography, and thus the title compound (345 mg) was obtained as a white solid.

¹H NMR (300 MHz, DMSO-d₆) δ ppm 2.63 (2H, t, J=6.4 Hz), 3.29 (2H, td, J=6.4, 5.7 Hz), 3.37 (2H, s), 3.80 (2H, s), 4.87 (2H, q, J=8.9 Hz), 7.21 (2H, d, J=9.0 Hz), 7.33 (2H, d, J=9.0 Hz), 8.38 (1H, t, J=5.7 Hz), 11.00 (1H, s).

### Example 329

### 2-[(2-Cyclopropylethyl)sulfanyl]-3-[4-(2,2,2-trifluoroethoxy)phenyl]-5,7-dihydro-3H-pyrrolo[2,3-d]pyrimidine-4,6-dione

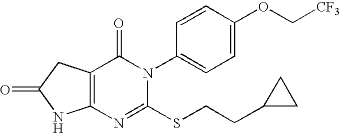

A mixture of 2-thioxo-3-[4-(2,2,2-trifluoroethoxy)phenyl]-2,3,5,7-tetrahydro-1H-pyrrolo[2,3-d]pyrimidine-4,6-dione (300 mg) obtained by the method of Example 46, or a method pursuant to thereto, a 1 M aqueous solution of sodium hydrogen carbonate (840 μl), 2-cyclopropylethyl 4-methylbenzenesulfonate (221 mg) obtained by a method described in a published document, WO 06/34312, or a method pursuant to thereto, and acetonitrile (8.5 ml) was heated to reflux for 2 hours. The reaction mixture was returned to room temperature, and then was diluted with ethyl acetate (80 ml). The dilution was washed with water and saturated brine, dried over anhydrous magnesium sulfate, and then concentrated under reduced pressure. The resulting residue was purified by chromatography, and thus the title compound (329 mg) was obtained as a white solid.

¹H NMR (300 MHz, DMSO-d₆) δ ppm 0.04-0.13 (2H, m), 0.37 (1H, dd, J=5.7, 4.2 Hz), 0.40 (1H, dd, J=5.7, 4.2 Hz), 0.63-0.79 (1H, m), 1.47 (2H, ddd, J=8.7, 7.2, 6.1 Hz), 3.09 (2H, dd, J=8.7, 6.1 Hz), 3.36 (2H, s), 4.85 (2H, q, J=8.9 Hz), 7.18 (2H, d, J=9.1 Hz), 7.29 (2H, d, J=9.1 Hz), 11.07 (1H, s).

### Example 330

### 2-[(2,2-Dimethylpropyl)sulfanyl]-3-[4-(2,2,2-trifluoroethoxy)phenyl]-5,7-dihydro-3H-pyrrolo[2,3-d]pyrimidine-4,6-dione

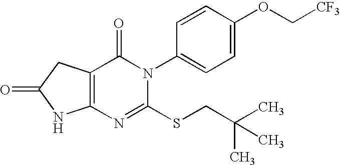

A mixture of 2-thioxo-3-[4-(2,2,2-trifluoroethoxy)phenyl]-2,3,5,7-tetrahydro-1H-pyrrolo[2,3-d]pyrimidine-4,6-dione (300 mg) obtained by the method of Example 46, or a method pursuant to thereto, a 1 M aqueous solution of sodium hydrogen carbonate (840 μl), 1-iodo-2,2-dimethylpropane (556 μl) and acetonitrile (8.5 ml) was heated to reflux for 3 hours. The reaction mixture was returned to room temperature, and then was diluted with ethyl acetate (80 ml). The dilution was washed with water and saturated brine, dried over anhydrous magnesium sulfate, and then concentrated under reduced pressure. The resulting residue was purified by chromatography, and thus the title compound (273 mg) was obtained as a white solid.

¹H NMR (300 MHz, DMSO-d₆) δ ppm 0.91 (9H, s), 3.10 (2H, s), 3.36 (2H, s), 4.87 (2H, q, J=8.9 Hz), 7.20 (2H, d, J=9.0 Hz), 7.31 (2H, d, J=9.0 Hz), 11.04 (1H, s).

### Example 331

### 2-(Benzylsulfanyl)-3-[4-(2,2,2-trifluoroethoxy)phenyl]-5,7-dihydro-3H-pyrrolo[2,3-d]pyrimidine-4,6-dione

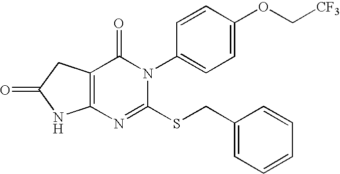

A mixture of 2-thioxo-3-[4-(2,2,2-trifluoroethoxy)phenyl]-2,3,5,7-tetrahydro-1H-pyrrolo[2,3-d]pyrimidine-4,6-dione (300 mg) obtained by the method of Example 46, or a method pursuant to thereto, a 1 M aqueous solution of sodium hydrogen carbonate (840 μl), benzyl bromide (131 μl) and acetonitrile (8.5 ml) was stirred for 30 minutes at 60° C. The reaction mixture was returned to room temperature, and then was diluted with ethyl acetate (80 ml). The dilution was washed with water and saturated brine, dried over anhydrous magnesium sulfate, and then concentrated under reduced pressure. The resulting residue was purified by chromatography, washed with diethyl ether, and thus the title compound (348 mg) was obtained as a white solid.

¹H NMR (300 MHz, DMSO-d₆) δ ppm 3.38 (2H, s), 4.30 (2H, s), 4.83 (2H, q, J=8.9 Hz), 7.16 (2H, d, J=9.0 Hz), 7.21-7.34 (5H, m), 7.35-7.46(2H, m), 11.16 (1H, s).

### Example 332

### 2-(Cyclopentylsulfanyl)-3-[4-(2,2,2-trifluoroethoxy)phenyl]-5,7-dihydro-3H-pyrrolo[2,3-d]pyrimidine-4,6-dione

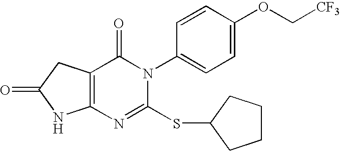

A mixture of 2-thioxo-3-[4-(2,2,2-trifluoroethoxy)phenyl]-2,3,5,7-tetrahydro-1H-pyrrolo[2,3-d]pyrimidine-4,6-dione (300 mg) obtained by the method of Example 46, or a method pursuant to thereto, a 1 M aqueous solution of sodium hydrogen carbonate (840 μl), iodocyclopentane (486 μl) and acetonitrile (8.5 ml) was heated to reflux for 2 hours. The reaction mixture was returned to room temperature, and then was diluted with ethyl acetate (80 ml). The dilution was washed with water and saturated brine, dried over anhydrous magnesium sulfate, and then concentrated under reduced pressure. The resulting residue was purified by chromatography, and thus the title compound (334 mg) was obtained as a white solid.

¹H NMR (300 MHz, DMSO-d₆) δ ppm 1.37-1.70 (6H, m), 2.04-2.21 (2H, m), 3.36 (2H, s), 3.83 (1H, quin, J=7.2 Hz), 4.85 (2H, q, J=8.8 Hz), 7.17 (2H, d, J=9.0 Hz), 7.29 (2H, d, J=9.0 Hz), 11.04 (1H, s).

### Example 333

### 2-{[(3-Methyloxetan-3-yl)methyl]sulfanyl}-3-[4-(2,2,2-trifluoroethoxy)phenyl]-5,7-dihydro-3H-pyrrolo[2,3-d}pyrimidine-4,6-dione

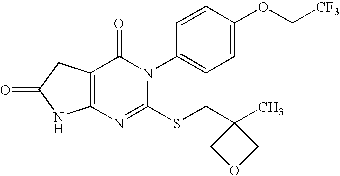

A mixture of 3-[4-(2,2,2-trifluoroethoxy)phenyl]-2-thioxo-2,3,5,7-tetrahydro-1H-pyrrolo[2,3-d]pyrimidine-4,6-dione (500 mg) obtained by the method of Example 46, or a method pursuant to thereto, 3-(chloromethyl)-3-methyloxetane (500 μl), a 1 M aqueous solution of sodium hydrogen carbonate (1.5 ml) and acetonitrile (20 ml) was stirred overnight at room temperature and for 3 hours at 60° C. The resulting mixture was cooled to room temperature. Then, water, ethyl acetate and a 5% aqueous solution of citric acid were added thereto, and the mixture was extracted with ethyl acetate. The organic layer was washed with water and saturated brine, dried over anhydrous sodium sulfate, and then concentrated under reduced pressure. The residue was purified by chromatography, and then was recrystallized from ethyl acetate. Thus, the title compound (271 mg) was obtained as a white solid.

¹H NMR (300 MHz, DMSO-d₆) δ ppm 1.25 (3H, s), 3.37 (2H, s), 3.46 (2H, s), 4.18 (2H, d, J=6.1 Hz), 4.32 (2H, d, J=6.1 Hz), 4.86 (2H, q, J=9.1 Hz), 7.19 (2H, d, J=8.9 Hz), 7.32 (2H, d, J=8.9 Hz), 11.08 (1H, s).

### Example 334

### 3-[4-(2,2,3,3,3-Pentafluoropropoxy)phenyl]-2-(pentylsulfanyl)-5,7-dihydro-3H-pyrrolo[2,3-d]pyrimidine-4,6-dione

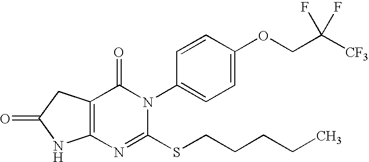

A mixture of 3-[4-(2,2,3,3,3-pentafluoropropoxy)phenyl]-2-thioxo-2,3,5,7-tetrahydro-1H-pyrrolo[2,3-d]pyrimidine-4,6-dione (3.00 g) obtained by the method of Example 47, or a method pursuant to thereto, 1-iodopentane (4 ml), a 1 M aqueous solution of sodium hydrogen carbonate (8 ml) and acetonitrile (100 ml) was stirred for one hour at 60° C. To the mixture, water, ethyl acetate and a 5% aqueous solution of citric acid were added, and the mixture was extracted with ethyl acetate. The organic layer was washed with water and saturated brine, dried over anhydrous sodium sulfate, and then concentrated under reduced pressure. The residue was purified by chromatography, and thus a pale purple solid (3.27 g) was obtained. This solid (160 mg) was recrystallized from a mixed solvent of ethyl acetate/hexane. Thus, the title compound (82 mg) was obtained as a white solid.

¹H NMR (300 MHz, DMSO-d₆) δ ppm 0.80-0.88 (3H, m), 1.22-1.31 (4H, m), 1.50-1.64 (2H, m), 3.03 (2H, t, J=7.3 Hz), 3.36 (2H, s), 4.93 (2H, t, J=13.2 Hz), 7.20 (2H, d, J=9.0 Hz), 7.30 (2H, d, J=9.0 Hz), 11.06 (1H, s).

### Example 335

### 3-[4-(2,2,3,3,3-Pentafluoropropoxy)phenyl]-2-(pentylsulfinyl)-5,7-dihydro-3H-pyrrolo[2,3-d]pyrimidine-4,6-dione

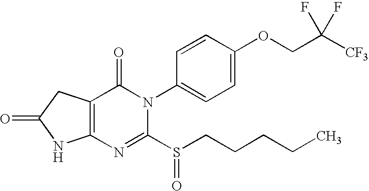

A solution of Oxone (registered trademark) monopersulfate compound (4.30 g) in water (20 ml) was added dropwise to a mixture at 60° C. of 3-[4-(2,2,3,3,3-pentafluoropropoxy)phenyl]-2-(pentylsulfanyl)-5,7-dihydro-3H-pyrrolo[2,3-d]pyrimidine-4,6-dione (3.11 g) obtained by the method of Example 334, or a method pursuant to thereto, and methanol (200 ml). The resulting mixture was stirred for one hour at 60° C., and then was concentrated under reduced pressure. Water was added to the residue, and then a solid generated therefrom was collected by filtration, washed with water and diethyl ether, and then dried, to obtain a dark red solid (2.72 g). This solid (120 mg) was recrystallized from ethyl acetate, and thus the title compound (53 mg) was obtained as a pale red solid.

¹H NMR (300 MHz, DMSO-d₆) δ ppm 0.79 (3H, t, J=6.8 Hz), 1.04-1.22 (4H, m), 1.45-1.59 (2H, m), 2.65-2.79 (1H, m), 2.86-2.99 (1H, m), 3.49 (2H, s), 4.94 (2H, t, J=13.4 Hz), 7.18-7.29 (2H, m), 7.39-7.47 (1H, m), 7.55-7.63 (1H, m), 11.44(1H, s).

### Example 336

### 2-[(4-Fluorophenyl)sulfanyl]-3-[4-(2,2,2-trifluoroethoxy)phenyl]-5,7-dihydro-3H-pyrrolo[2,3-d]pyrimidine-4,6-dione

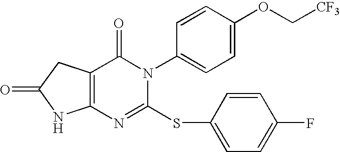

A mixture of 4-fluorobenzene thiol (825 μl), sodium hydride (60% in oil, 232 mg) and tetrahydrofuran (5 ml) was added to a mixture of 2-(methylsulfinyl)-3-[4-(2,2,2-trifluoroethoxy)phenyl]-5,7-dihydro-3H-pyrrolo[2,3-d]pyrimidine-4,6-dione (1.50 g) and tetrahydrofuran (100 ml) in an ice water bath. The mixture was stirred for 30 minutes in an ice water bath. Then, water, ethyl acetate and a 5% aqueous solution of citric acid were added thereto, and the mixture was extracted with ethyl acetate. The organic layer was washed with water and saturated brine, dried over anhydrous sodium sulfate, and then concentrated under reduced pressure. To the residue, and hexane was added. Then, a solid generated therefrom was collected by filtration, purified by chromatography, and then recrystallized from a mixed solvent of ethyl acetate/hexane. Thus, the title compound (289 mg) was obtained as a yellow solid.

¹H NMR (300 MHz, DMSO-d₆) δ ppm 3.33 (2H, s), 4.88 (2H, q, J=9.0 Hz), 7.25 (2H, d, J=9.1 Hz), 7.32 (2H, t, J=8.9 Hz), 7.45 (2H, d, J=9.1 Hz), 7.58 (2H, dd, J=9.1, 5.3 Hz), 10.83 (1H, s).

### Example 337

### 2-(Tert-butylsulfanyl)-3-[4-(2,2,2-trifluoroethoxy)phenyl]-5,7-dihydro-3H-pyrrolo[2,3-d]pyrimidine-4,6-dione

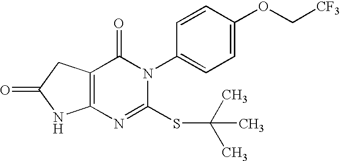

A mixture of 2-methylpropan-2-thiol (1 ml), sodium hydride (60% in oil, 464 mg) and tetrahydrofuran (10 ml) was added to a mixture of 2-(methylsulfinyl)-3-[4-(2,2,2-trifluoroethoxy)phenyl]-5,7-dihydro-3H-pyrrolo[2,3-d]pyrimidine-4,6-dione (1.50 g) and tetrahydrofuran (100 ml) in an ice water bath. The mixture was stirred for 30 minutes in an ice water bath and for 30 minutes at room temperature. Then, water, ethyl acetate and a 5% aqueous solution of citric acid were added thereto, and the mixture was extracted with ethyl acetate. The organic layer was washed with water and saturated brine, dried over anhydrous sodium sulfate, and then concentrated under reduced pressure. The residue was purified by chromatography, and then was recrystallized from a mixed solvent of ethyl acetate/hexane. Thus, the title compound (595 mg) was obtained as a white solid.

¹H NMR (300 MHz, DMSO-d₆) δ ppm 1.51 (9H, s), 3.36 (2H, s), 4.85 (2H, q, J=9.1 Hz), 7.16 (2H, d, J=9.1 Hz), 7.24 (2H, d, J=9.1 Hz), 11.05 (1H, s).

### Example 338

### 2-[2-Fluoro-1-(fluoromethyl)ethoxy]-3-[4-(2,2,2-trifluoroethoxy)phenyl]-5,7-dihydro-3H-pyrrolo[2,3-d]pyrimidine-4,6-dione

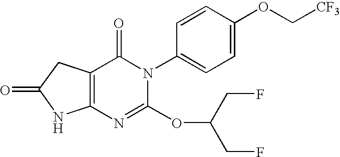

A mixture of 1,3-difluoropropan-2-ol (5 ml), sodium hydride (60% in oil, 464 mg) and tetrahydrofuran (20 ml) was added to a mixture of 2-(methylsulfinyl)-3-[4-(2,2,2-trifluoroethoxy)phenyl]-5,7-dihydro-3H-pyrrolo[2,3-d]pyrimidine-4,6-dione (1.50 g) and tetrahydrofuran (100 ml) in an ice water bath. The mixture was stirred in an ice water bath for one hour. Then, water, ethyl acetate and a 5% aqueous solution of citric acid were added thereto, and the mixture was extracted with ethyl acetate. The organic layer was washed with water and saturated brine, dried over anhydrous sodium sulfate, and then concentrated under reduced pressure. The residue was purified by chromatography, and then was recrystallized from a mixed solvent of ethyl acetate/hexane. Thus, the title compound (454 mg) was obtained as a white solid.

¹H NMR (300 MHz, DMSO-d₆) δ ppm 3.35 (2H, s), 4.41-4.91 (6H, m), 5.41-5.64 (1H, m), 7.14 (2H, d, J=8.9 Hz), 7.24 (2H, d, J=8.9 Hz), 11.08 (1H, s).

### Example 339

### 1-[({4,6-Dioxo-3-[4-(2,2,2-trifluoroethoxy)phenyl]-4,5,6,7-tetrahydro-3H-pyrrolo[2,3-d]pyrimidin-2-yl}sulfanyl)methyl]cyclopropanecarbonitrile

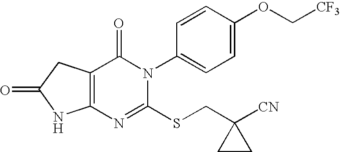

A mixture of 2-thioxo-3-[4-(2,2,2-trifluoroethoxy)phenyl]-2,3,5,7-tetrahydro-1H-pyrrolo[2,3-d]pyrimidine-4,6-dione (300 mg) obtained by the method of Example 46, or a method pursuant to thereto, a 1 M aqueous solution of sodium hydrogen carbonate (840 μl), 1-(bromomethyl)cyclopropane carbonitrile (269 mg) obtained by a method described in a published document, Journal of the American Chemical Society (J. Am. Chem. Soc.), Vol. 110, p. 8050 (1988), or a method pursuant to thereto, and acetonitrile (8.5 ml) was stirred for one hour at 80° C. The reaction mixture was returned to room temperature, and then was diluted with ethyl acetate (80 ml). The dilution was washed with water and saturated brine, dried over anhydrous magnesium sulfate, and concentrated under reduced pressure. The resulting residue was purified by chromatography, and thus the title compound (342 mg) was obtained as a white solid.

¹H NMR (300 MHz, DMSO-d₆) δ ppm 1.12-1.18 (2H, m), 1.26-1.33 (2H, m), 3.38 (2H, s), 3.38 (2H, s), 4.88 (2H, q, J=8.9 Hz), 7.22 (2H, d, J=9.1 Hz), 7.33 (2H, d, J=9.1 Hz), 11.11 (1H, s).

### Example 340

### 2-[(1-Ethylpropyl)sulfanyl]-3-[4-(2,2,2-trifluoroethoxy)phenyl]-5,7-dihydro-3H-pyrrolo[2,3-d]pyrimidine-4,6-dione

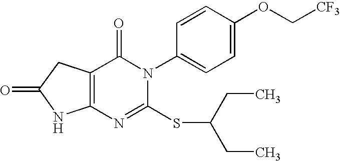

A mixture of 2-thioxo-3-[4-(2,2,2-trifluoroethoxy)phenyl]-2,3,5,7-tetrahydro-1H-pyrrolo[2,3-d]pyrimidine-4,6-dione (300 mg) obtained by the method of Example 46, or a method pursuant to thereto, a 1 M aqueous solution of sodium hydrogen carbonate (840 μl), 3-bromopentane (1.05 ml) and acetonitrile (8.5 ml) was heated to reflux for 3 hours. The reaction mixture was returned to room temperature, and then was diluted with ethyl acetate (80 ml). The dilution was washed with water and saturated brine, dried over anhydrous magnesium sulfate, and then concentrated under reduced pressure. The resulting residue was purified by chromatography, and thus the title compound (358 mg) was obtained as a white solid.

¹H NMR (300 MHz, DMSO-d₆) δ ppm 0.88 (6H, t, J=7.2 Hz), 1.46-1.77 (4H, m), 3.36 (2H, s), 3.62-3.75 (1H, m), 4.86 (2H, q, J=8.9 Hz), 7.18 (2H, d, J=9.1 Hz), 7.29 (2H, d, J=9.1 Hz), 11.03 (1H, s).

### Example 341

### 3-[4-(2,2,2-Trifluoroethoxy)phenyl]-2-(3,3,3-trifluoropropoxy)-5,7-dihydro-3H-pyrrolo[2,3-d]pyrimidine-4,6-dione

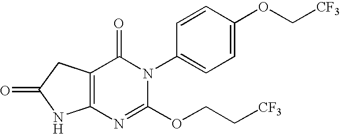

A mixture of 3,3,3-trifluoropropan-1-ol (3 g), sodium hydride (60% in oil, 464 mg) and tetrahydrofuran (50 ml) was added to a mixture of 2-(methylsulfinyl)-3-[4-(2,2,2-trifluoroethoxy)phenyl]-5,7-dihydro-3H-pyrrolo[2,3-d]pyrimidine-4,6-dione (1.50 g) and tetrahydrofuran (50 ml) in an ice water bath. The mixture was stirred in an ice water bath for one hour. Then, water, ethyl acetate and a 5% aqueous solution of citric acid were added thereto, and the mixture was extracted with ethyl acetate. The organic layer was washed with water and saturated brine, dried over anhydrous sodium sulfate, and then concentrated under reduced pressure. The residue was purified by chromatography, and then was recrystallized from a mixed solvent of ethyl acetate/hexane. Thus, the title compound (165 mg) was obtained as a white solid.

¹H NMR (300 MHz, DMSO-d₆) δ ppm 2.54-2.78 (2H, m), 3.34 (2H, s), 4.49 (2H, t, J=5.9 Hz), 4.82 (2H, q, J=8.7 Hz), 7.12 (2H, d, J=9.1 Hz), 7.20 (2H, d, J=9.1 Hz), 11.08 (1H, s).

### Example 342

### 3-[4-(2,2,2-Trifluoroethoxy)phenyl]-2-[4-(trifluoromethyl)phenoxy]-5,7-dihydro-3H-pyrrolo[2,3-d]pyrimidine-4,6-dione

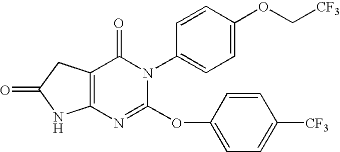

A mixture of 4-(trifluoromethyl)phenol (3.13 g), sodium hydride (60% in oil, 464 mg) and tetrahydrofuran (50 ml) was added to a mixture of 2-(methylsulfinyl)-3-[4-(2,2,2-trifluoroethoxy)phenyl]-5,7-dihydro-3H-pyrrolo[2,3-d]pyrimidine-4,6-dione (1.50 g) and tetrahydrofuran (50 ml) in an ice water bath. The mixture was stirred in an ice water bath for one hour. Then, water, ethyl acetate and a 5% aqueous solution of citric acid were added thereto, and the mixture was extracted with ethyl acetate. The organic layer was washed with water and saturated brine, dried over anhydrous sodium sulfate, and then concentrated under reduced pressure. The residue was purified by chromatography, and then was recrystallized from a mixed solvent of ethyl acetate/hexane. Thus, the title compound (571 mg) was obtained as a white solid.

¹H NMR (300 MHz, DMSO-d₆) δ ppm 3.37 (2H, s), 4.83 (2H, q, J=9.1 Hz), 7.19 (2H, d, J=9.1 Hz), 7.45-7.57 (4H, m), 7.84 (2H, d, J=8.7 Hz), 10.99 (1H, s).

### Example 343

### 2-(4-Chlorophenoxy)-3-[4-(2,2,2-trifluoroethoxy)phenyl]-5,7-dihydro-3H-pyrrolo[2,3-d]pyrimidine-4,6-dione

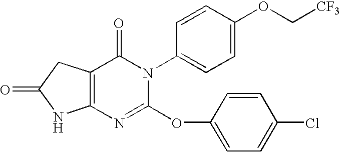

A mixture of 4-chlorophenol (1.49 g), sodium hydride (60% in oil, 464 mg) and tetrahydrofuran (50 ml) was added to a mixture of 2-(methylsulfinyl)-3-[4-(2,2,2-trifluoroethoxy)phenyl]-5,7-dihydro-3H-pyrrolo[2,3-d]pyrimidine-4,6-dione (1.50 g) and tetrahydrofuran (50 ml) in an ice water bath. The mixture was stirred in an ice water bath for one hour. Then, water, ethyl acetate and a 5% aqueous solution of citric acid were added thereto, and the mixture was extracted with ethyl acetate. The organic layer was washed with water and saturated brine, dried over anhydrous sodium sulfate, and then concentrated under reduced pressure. The residue was purified by chromatography, and then was recrystallized from a mixed solvent of ethyl acetate/hexane. Thus, the title compound (295 mg) was obtained as a white solid.

¹H NMR (300 MHz, DMSO-d₆) δ ppm 3.35 (2H, s), 4.83 (2H, q, J=9.1 Hz), 7.18 (2H, d, J=9.1 Hz), 7.30 (2H, d, J=9.1 Hz), 7.42-7.55 (4H, m), 10.99 (1H, s).

### Example 344

### 2-(4-Chlorophenoxy)-3-[4-(2,2,3,3,3-pentafluoropropoxy)phenyl]-5,7-dihydro-3H-pyrrolo[2,3-d]pyrimidine-4,6-dione

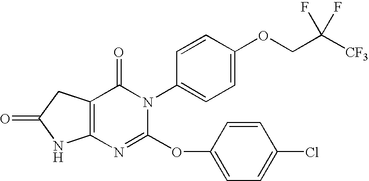

A mixture of 4-chlorophenol (1.49 g), sodium hydride (60% in oil, 464 mg) and tetrahydrofuran (30 ml) was added to a mixture of 2-(methylsulfinyl)-3-[4-(2,2,3,3,3-pentafluoropropoxy)phenyl]-5,7-dihydro-3H-pyrrolo[2,3-d]pyrimidine-4,6-dione (1.50 g) obtained by the method of Example 304, or a method pursuant to thereto, and tetrahydrofuran (50 ml) in an ice water bath. The mixture was stirred in an ice water bath for one hour. Then, water, ethyl acetate and a 5% aqueous solution of citric acid were added thereto, and the mixture was extracted with ethyl acetate. The organic layer was washed with water and saturated brine, dried over anhydrous sodium sulfate, and then concentrated under reduced pressure. The residue was purified by chromatography, and then was recrystallized from a mixed solvent of ethyl acetate/hexane. Thus, the title compound (425 mg) was obtained as a white solid.

¹H NMR (300 MHz, DMSO-d₆) δ ppm 3.35 (2H, s), 4.90 (2H, t, J=13.3 Hz), 7.20 (2H, d, J=8.7 Hz), 7.30 (2H, d, J=8.7 Hz), 7.42-7.58 (4H, m), 10.99 (1H, s).

### Example 345

### 3-[4-(2,2,2-Trifluoroethoxy)phenyl]-2-[4-(trifluoromethoxy)phenoxy]-5,7-dihydro-3H-pyrrolo[2,3-d]pyrimidine-4,6-dione

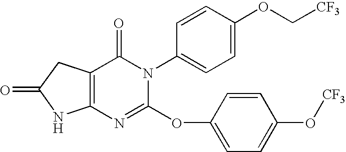

A mixture of 4-(trifluoromethoxy)phenol (1.00 g), sodium hydride (60% in oil, 200 mg) and tetrahydrofuran (50 ml) was added to a mixture of 2-(methylsulfinyl)-3-[4-(2,2,2-trifluoroethoxy)phenyl]-5,7-dihydro-3H-pyrrolo[2,3-d]pyrimidine-4,6-dione (1.50 g) and tetrahydrofuran (50 ml) in an ice water bath. The mixture was stirred in an ice water bath for one hour. Then, water, ethyl acetate and a 5% aqueous solution of citric acid were added thereto, and the mixture was extracted with ethyl acetate. The organic layer was washed with water and saturated brine, dried over anhydrous sodium sulfate, and then concentrated under reduced pressure. The residue was purified by chromatography, and then was recrystallized from a mixed solvent of ethyl acetate/hexane. Thus, the title compound (185 mg) was obtained as a yellow solid.

¹H NMR (300 MHz, DMSO-d₆) δ ppm 3.36 (2H, s), 4.83 (2H, q, J=9.1 Hz), 7.19 (2H, d, J=9.1 Hz), 7.36-7.53 (6H, m), 11.00 (1H, s).

### Example 346

### 2-[(1-Ethylpropyl)sulfanyl]-3-[4-(2,2,3,3,3-pentafluoropropoxy)phenyl]-5,7-dihydro-3H-pyrrolo[2,3-d]pyrimidine-4,6-dione

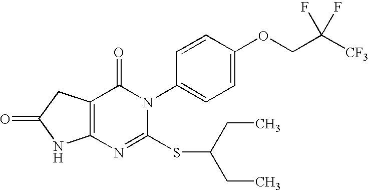

A mixture of 3-[4-(2,2,3,3,3-pentafluoropropoxy)phenyl]-2-thioxo-2,3,5,7-tetrahydro-1H-pyrrolo[2,3-d]pyrimidine-4,6-dione (550 mg) obtained by the method of Example 47, or a method pursuant to thereto, 3-bromopentane (5 ml), sodium iodide (5 mg), a 1 M aqueous solution of sodium hydrogen carbonate (1.35 ml) and acetonitrile (30 ml) was stirred for 4 hours at 80° C. The resulting mixture was cooled to room temperature. Then, water, ethyl acetate and a 5% aqueous solution of citric acid were added thereto, and the mixture was extracted with ethyl acetate. The organic layer was washed with water and saturated brine, dried over anhydrous sodium sulfate, and then concentrated under reduced pressure. The residue was purified by chromatography, and then was recrystallized from a mixed solvent of ethyl acetate/hexane. Thus, the title compound (547 mg) was obtained as a white solid.

¹H NMR (300 MHz, DMSO-d₆) δ ppm 0.89 (6H, t, J=7.4 Hz), 1.44-1.78 (4H, m), 3.35 (2H, s), 3.60-3.77 (1H, m), 4.93 (2H, t, J=13.3 Hz), 7.20 (2H, d, J=9.1 Hz), 7.30 (2H, d, J=9.1 Hz), 11.03 (1H, s).

### Example 347

### 3-[4-(2,2,3,3,3-Pentafluoropropoxy)phenyl]-2-[(2,2,2-trifluoroethyl)sulfanyl]-5,7-dihydro-3H-pyrrolo[2,3-d]pyrimidine-4,6-dione

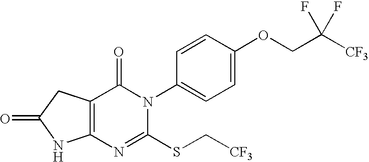

A mixture of 3-[4-(2,2,3,3,3-pentafluoropropoxy)phenyl]-2-thioxo-2,3,5,7-tetrahydro-1H-pyrrolo[2,3-d]pyrimidine-4,6-dione (550 mg) obtained by the method of Example 47, or a method pursuant to thereto, 1,1,1-trifluoro-2-iodoethane (5 ml), a 1 M aqueous solution of sodium hydrogen carbonate (1.35 ml) and acetonitrile (30 ml) was stirred overnight at 90° C. The resulting mixture was cooled to room temperature. Then, water, ethyl acetate and a 5% aqueous solution of citric acid were added thereto, and the mixture was extracted with ethyl acetate. The organic layer was washed with water and saturated brine, dried over anhydrous sodium sulfate, and then concentrated under reduced pressure. The residue was purified by chromatography, and then was recrystallized from a mixed solvent of ethyl acetate/hexane. Thus, the title compound (330 mg) was obtained as a pale yellow solid.

^{I}H NMR (300 MHz, DMSO-d₆) δ ppm 3.40 (2H, s), 4.18 (2H, q, J=10.3 Hz), 4.95 (2H, t, J=13.6 Hz), 7.24 (2H, d, J=8.9 Hz), 7.38 (2H, d, J=8.9 Hz), 11.16 (1H, s).

### Example 348

### 2-(Methylsulfanyl)-3-[4(2,2,2-trifluoroethoxy)phenyl]-5,7-dihydro-3H-pyrrolo[2,3-d]pyrimidine-4,6-dione

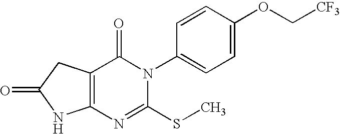

2-Thioxo-3-[4-(2,2,2-trifluoroethoxy)phenyl]-2,3,5,7-tetrahydro-1H-pyrrolo[2,3-d]pyrimidine-4,6-dione (665 mg) was suspended in acetonitrile (18 ml) and a 1 M aqueous solution of sodium hydrogen carbonate (1.86 ml) and methyl iodide (0.582 ml) were added to the suspension. The mixture was stirred at 60° C. for 30 minutes, cooled, diluted with ethyl acetate (150 ml), washed with water and saturated brine, dried over anhydrous magnesium sulfate and concentrated under reduced pressure. The resultant crude product was purified by chromatography to give the title compound (550 mg) as a purple solid.

¹H NMR (300 MHz, DMSO-d₆) δ ppm 2.41 (3H, s), 3.36 (2H, s), 4.86 (2H, q, J=8.9 Hz), 7.20 (2H, d, J=9.1 Hz), 7.31 (2H, d, J=9.1 Hz), 11.10 (1H, s).

### Example 349

### 2-(Methylsulfinyl)-3-[4-(2,2,2-trifluoroethoxy)phenyl]-5,7-dihydro-3H-pyrrolo[2,3-d]pyrimidine-4,6-dione

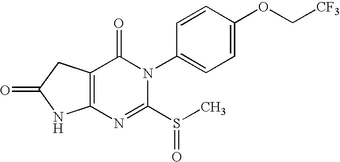

2-(Methylsulfanyl)-3-[4-(2,2,2-trifluoroethoxy)phenyl]-5,7-dihydro-3H-pyrrolo[2,3-d]pyrimidine-4,6-dione (50 g) was suspended in acetic acid (500 ml) and to the suspension was added dropwise a suspension of Oxone (registered trademark) monopersulfate compound (99 g) in water (250 ml) over 10 minutes while maintained at 40° C. or below with water bath. The mixture was stirred at 38° C. to 45° C. for 60 minutes and poured into ice water (500 ml) and the mixture was stirred for 15 minutes with ice water cooling. The pale pink precipitates were collected by filtration, washed with water two times, washed with 50% acetonitrile/isopropyl ether (100 ml) two times, and dried to give the title compound (35.5 g) as a white powder.

¹HNMR (300 MHz, DMSO-d₆) δ ppm 2.69 (3H, s), 3.49 (2H, s), 4.87 (2H, q, J=8.8 Hz), 7.14-7.27 (2H, m), 7.38-7.46 (1H, m), 7.49-7.60 (1H, m), 11.44 (1H, s).

### Example 350

### 2-(2,2,3,3,3-Pentafluoropropoxy)-3-[4-(2,2,2-trifluoroethoxy)phenyl]-5,7-dihydro-3H-pyrrolo[2,3-d]pyrimidine-4,6-dione

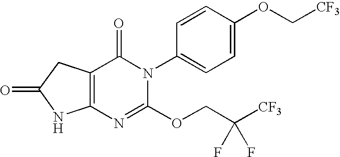

To a solution of 2,2,3,3,3-pentafluoropropan-1-ol (1.54 ml) in tetrahydrofuran (4 ml) was added dropwise 1,8-diazabicyclo[5.4.0]undec-7-ene (1.63 ml) with water bath, and the mixture was stirred for 5 minutes at room temperature. The mixture was added dropwise to a suspension of 2-(methylsulfinyl)-3-[4-(2,2,2-trifluoroethoxy)phenyl]-5,7-dihydro-3H-pyrrolo[2,3-d]pyrimidine-4,6-dione (2.0 g) in tetrahydrofran (14 ml) over 7 minutes with ice cooling. The mixture was washed in with tetrahydrofuran (2 ml), N,N-dimethylformamide (4 ml) was added dropwise to the mixture over 3 minutes. The mixture was stirred for an additional 10 minutes with ice cooling. To the reaction mixture was added 1 M hydrochloric acid (14 ml) and the mixture was diluted with ethyl acetate (50 ml). After separating the aqueous layer, the organic layer was washed with water three times, washed with saturated brine, dried over anhydrous magnesium sulfate, and concentrated under reduced pressure to give a pale purple solid. The solid was purified by chromatography, and then was washed with a mixed solvent of ethyl acetate/hexane, and thus the title compound (1.74 g) was obtained as a beige powder.

¹H NMR (300 MHz, DMSO-d₆) δ ppm 3.38 (2H, s), 4.83 (2H, q, J=8.9 Hz), 5.04 (2H, t, J=12.9 Hz), 7.15 (2H, d, J=9.1 Hz), 7.25 (2H, d, J=9.1 Hz), 11.18 (1H, s).

### Example 351

### 2-(2,2,2-Trifluoroethoxy)-3-[4-(2,2,2-trifluoroethoxy)phenyl]-5,7-dihydro-3H-pyrrolo[2,3-d]pyrimidine-4,6-dione

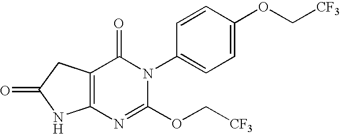

To a solution of 2,2,2-trifluoroethanol (29.3 ml) in tetrahydrofuran (100 ml), 1,8-diazabicyclo[5.4.0]undec-7-en (43.0 ml) was added dropwise with water bath, and the resulting mixture was stirred for 5 minutes at room temperature. This mixture was added dropwise over 7 minutes under ice cooling to a suspension of 2-(methylsulfinyl)-3-[4-(2,2,2-trifluoroethoxy)phenyl]-5,7-dihydro-3H-pyrrolo[2,3-d]pyrimidine-4,6-dione (52.6 g) in tetrahydrofuran (350 ml). The mixture was washed in with tetrahydrofuran (50 ml), and then N,N-dimethylformamide (100 ml) was added thereto dropwise over 3 minutes. Subsequently, the mixture was further stirred for 7 minutes under ice cooling. To the reaction mixture was added 1 M hydrochloric acid (300 ml), and the mixture was diluted with ethyl acetate (800 ml). The aqueous layer was removed therefrom, and the organic layer was washed with water three times, and then was washed with saturated brine. The resultant was dried over anhydrous magnesium sulfate, and then concentrated under reduced pressure to give a pale purple solid. The solid was purified by chromatography, and then was washed with a mixed solvent of ethyl acetate/hexane, and thus the title compound (44.2 g) was obtained as a beige powder.

¹H NMR (300 MHz, DMSO-d₆) δ ppm 3.37 (2H, s), 4.84 (2H, q, J=8.9 Hz), 4.97 (2H, q, J=8.7 Hz), 7.16 (2H, d, J=9.1 Hz), 7.27 (2H, d, J=9.1 Hz), 11.17 (1H, s).

### Example 352

### 2-(2,2,3,3,3-Pentafluoropropoxy)-3-[4-(2,2,2-trifluoroethoxy)phenyl]-5,7-dihydro-3H-pyrrolo[2,3-d]pyrimidine-4,6-dione

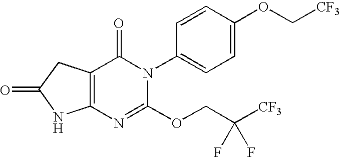

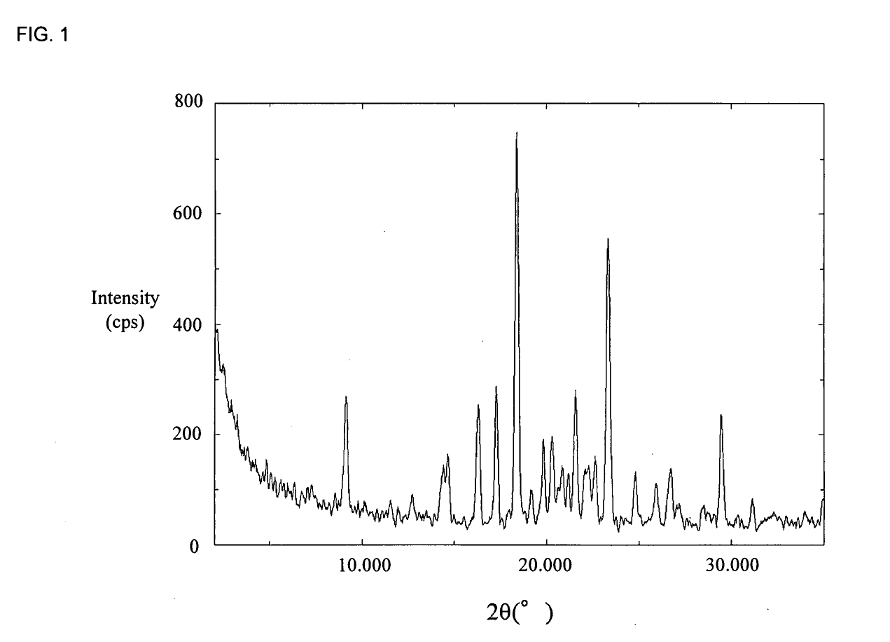

Ethyl acetate (0.3 ml) was added to 2-(2,2,3,3,3-pentafluoropropoxy)-3-[4-(2,2,2-trifluoroethoxy)phenyl]-5,7-dihydro-3H-pyrrolo[2,3-d]pyrimidine-4,6-dione (15 mg), and the mixture was heated to be dissolved. Then, heptane (0.3 ml) was added thereto, and the mixture was cooled to obtain crystals. The powder X-ray crystal diffraction data (main peaks) of the obtained crystals are shown in Table 1, and the powder X-ray crystal diffraction pattern of the obtained crystals is shown in FIG. 1, which were measured using Cu-Kα radiation (X-ray tube voltage: 40 KV; X-ray tube current: 50 mA) as a radiation source, and using RINT Ultima+2100 type powder X-ray diffractometer (manufactured by Rigaku Corporation).

TABLE 1
Powder X-ray crystal diffraction data (main peaks)

| Diffraction Angle: 2θ(°) | d value (Angstrom) |
|---|---|
| 9.12 | 9.69 |
| 16.3 | 5.43 |
| 18.4 | 4.82 |
| 19.8 | 4.48 |
| 21.6 | 4.12 |
| 23.3 | 3.81 |
| 29.5 | 3.03 |

### Example 353

### 2-(2,2,3,3,3-Pentafluoropropoxy)-3-[4-(2,2,2-trifluoroethoxy)phenyl]-5,7-dihydro-3H-pyrrolo[2,3-d]pyrimidine-4,6-dione

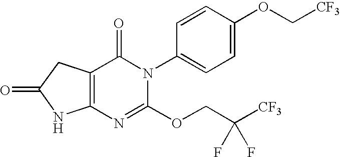

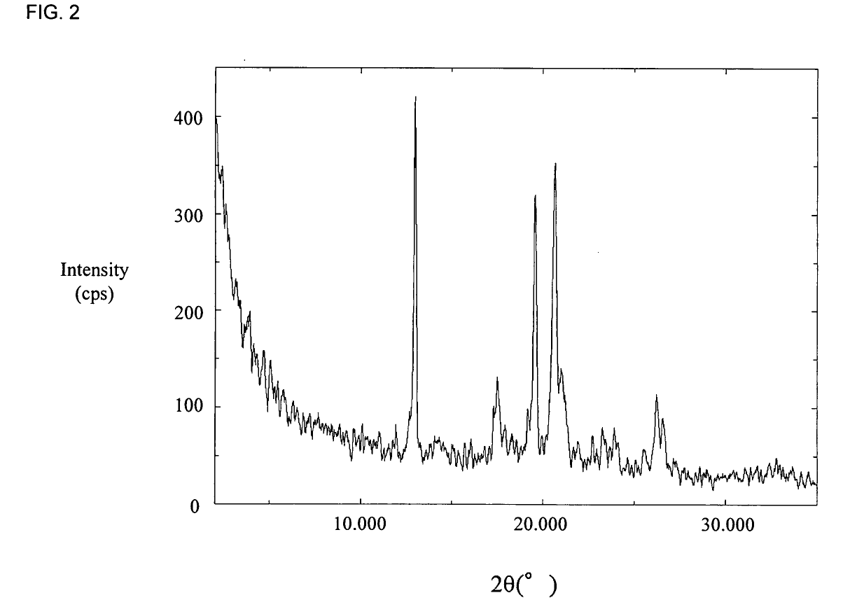

Methanol (0.2 ml) was added to 2-(2,2,3,3,3-pentafluoropropoxy)-3-[4-(2,2,2-trifluoroethoxy)phenyl]-5,7-dihydro-3H-pyrrolo[2,3-d]pyrimidine-4,6-dione (15 mg), and the mixture was heated to be dissolved. Then, diisopropyl ether (0.3 ml) was added thereto, and the mixture was cooled to obtain crystals. The powder X-ray crystal diffraction data (main peaks) of the obtained crystals are shown in Table 2, and the powder X-ray crystal diffraction pattern of the obtained crystals is shown in FIG. 2, which were measured using Cu-Kα radiation (X-ray tube voltage: 40 KV; X-ray tube current: 50 mA) as a radiation source, and using RINT Ultima+2100 type powder X-ray diffractometer (manufactured by Rigaku Corporation).

TABLE 2
Powder X-ray crystal diffraction data (main peaks)

| Diffraction Angle: 2θ(°) | d value (Angstrom) |
|---|---|
| 13.0 | 6.81 |
| 17.5 | 5.06 |
| 19.6 | 4.53 |
| 20.6 | 4.30 |
| 21.0 | 4.22 |

### Preparation Example 1

| | |
|---|---|
| (1) Compound of Example 1 | 10.0 g |
| (2) Lactose | 70.0 g |
| (3) Corn starch | 50.0 g |
| (4) Soluble starch | 7.0 g |
| (5) Magnesium stearate | 3.0 g |

10.0 g of the compound of Example 1 and 3.0 g of magnesium stearate are granulized with an aqueous solution of soluble starch (70 ml, i.e., 7.0 g of soluble starch). Then, the granules are dried, and are admixed with 70.0 g of lactose and 50.0 g of corn starch (lactose, corn starch, soluble starch and magnesium stearate are all in compliance with Japanese Pharmacopoeia, 14th Edition). The mixture is compressed to obtain a tablet.

### Test Example 1

An inhibitory activity of a test compound on delta-5-desaturase was measured according to the method described herein below. To a DMSO solution of the test compound which had been previously prepared, secondary dilution was carried out by using a buffer (300 mM NaH₂PO₄ [pH 7.4], 450 mM KCl, 30 mM NaF, 9 mM MgCl₂, 4.5 mM glutathione [reduced form], 0.3% BSA [fatty acid free, SIGMA]). This diluent compound (10 μl) was added in a 96-well deep well block made of polypropylene, and then the microsomal fraction of a rat river (10 μl), which had been diluted to 3 mg/ml by using a microsome buffer (10 mM Tris-HCl [pH 7.4], 1 mM EDTA, and 250 mM sucrose), was added thereto. The enzyme reaction was initiated by adding 10 μl of 9 mM NADH, 9 mM ATP, 0.9 mM CoA and 10 μCi/ml (8E,11E,14E)-(1-¹⁴C)eicosa-8,11,14-trienoic acid (PerkinElmer Inc.). The enzyme reaction was carried out at room temperature for 120 minutes, and then terminated by addition of 2.5 M NaOH (10 μl). Upon the completion of the reaction, the plate was covered with plate seal and incubated overnight in a dry heater of which temperature was set at 55° C. for saponification. Based on Bligh & Dyer method which had been described in a known document, Canadian Journal of Biochemistry and Physiology (Can. J. Biochem. Physiol.), Vol. 37, page 911, 1959), the solvent extraction of fatty acid was carried out by adding 200 μl of formic acid:methanol:chloroform (1:6:3), keeping the mixture as a single layer for a while, stirring the mixture to a sufficient level, and adding 120 μl of pure water to separate the mixture into two layers. 10 μl of the bottom chloroform layer was spotted on a reverse phase TLC plate (RP-18, 1154230001, Merck Japan, Ltd.) and then developed with acetonitrile:pure water:acetic acid (95:4.5:0.5). The TLC plate obtained after drying was transferred to an Imaging Plate (Fuji Photo Film Co., Ltd.) for more than 5 hours. Detection was carried out by using BAS-5000 (Fuji Photo Film Co., Ltd.). Numerization of thus-obtained image was carried out by using Multi Gauge Ver 2.3 (Fuji Photo Film Co., Ltd.) and an inhibitory ratio (%) of the test compound at 10 μM on delta-5-desaturase was obtained.

The results are summarized in Table 3.

TABLE 3
Inhibitory activity of delta-5-desaturase

| Example number | Inhibitory activity at 10 μM (%) |
|---|---|
| 60 | 99 |
| 62 | 102 |
| 63 | 98 |
| 65 | 97 |
| 66 | 101 |
| 67 | 96 |
| 74 | 101 |
| 75 | 99 |
| 76 | 99 |
| 77 | 100 |
| 78 | 101 |
| 79 | 99 |
| 80 | 98 |
| 81 | 97 |
| 82 | 101 |
| 85 | 99 |
| 86 | 99 |
| 87 | 99 |
| 88 | 100 |
| 90 | 99 |
| 91 | 100 |
| 92 | 102 |
| 93 | 101 |
| 95 | 100 |
| 97 | 100 |
| 98 | 100 |
| 99 | 94 |
| 100 | 100 |
| 103 | 100 |
| 104 | 100 |
| 105 | 100 |
| 106 | 102 |
| 107 | 100 |
| 108 | 101 |
| 109 | 101 |
| 110 | 102 |
| 111 | 98 |
| 112 | 102 |
| 113 | 102 |
| 115 | 103 |
| 116 | 101 |
| 119 | 102 |
| 120 | 101 |
| 121 | 100 |
| 123 | 101 |
| 127 | 102 |
| 129 | 101 |
| 130 | 100 |
| 131 | 108 |
| 132 | 107 |
| 134 | 100 |
| 135 | 100 |
| 136 | 99 |
| 137 | 99 |
| 139 | 101 |
| 141 | 100 |
| 142 | 98 |
| 143 | 101 |
| 144 | 101 |
| 147 | 98 |
| 153 | 98 |
| 154 | 99 |
| 155 | 99 |
| 157 | 101 |
| 158 | 99 |
| 159 | 99 |
| 162 | 103 |
| 163 | 99 |
| 164 | 101 |
| 165 | 99 |
| 166 | 101 |
| 170 | 101 |
| 171 | 98 |
| 172 | 99 |
| 173 | 97 |
| 174 | 100 |
| 175 | 100 |
| 176 | 98 |
| 177 | 100 |
| 183 | 99 |
| 185 | 97 |
| 186 | 100 |
| 188 | 98 |
| 189 | 105 |
| 191 | 101 |
| 192 | 101 |
| 193 | 100 |
| 194 | 103 |
| 195 | 101 |
| 207 | 101 |
| 208 | 100 |
| 209 | 99 |
| 212 | 98 |
| 213 | 100 |
| 214 | 101 |
| 215 | 102 |
| 217 | 99 |
| 219 | 102 |
| 221 | 104 |
| 222 | 101 |
| 223 | 103 |
| 224 | 100 |
| 225 | 100 |
| 226 | 102 |
| 227 | 99 |
| 231 | 101 |
| 232 | 96 |
| 237 | 98 |
| 239 | 101 |
| 244 | 101 |
| 246 | 101 |
| 247 | 103 |
| 248 | 101 |
| 249 | 100 |
| 251 | 97 |
| 252 | 96 |
| 253 | 98 |
| 254 | 98 |
| 255 | 101 |
| 256 | 104 |
| 264 | 101 |
| 265 | 98 |
| 267 | 100 |
| 269 | 99 |
| 270 | 99 |
| 274 | 100 |
| 275 | 99 |
| 277 | 94 |
| 283 | 101 |
| 285 | 96 |
| 286 | 102 |
| 287 | 98 |
| 289 | 101 |
| 291 | 99 |
| 296 | 101 |
| 297 | 101 |
| 298 | 101 |
| 299 | 100 |
| 300 | 101 |
| 301 | 99 |
| 302 | 94 |
| 303 | 98 |
| 306 | 99 |
| 307 | 99 |
| 309 | 98 |
| 310 | 99 |
| 311 | 98 |
| 312 | 97 |
| 313 | 102 |
| 315 | 99 |
| 316 | 103 |
| 318 | 103 |
| 319 | 104 |
| 322 | 101 |
| 323 | 102 |
| 324 | 101 |
| 325 | 105 |
| 326 | 102 |
| 328 | 97 |
| 329 | 101 |
| 336 | 101 |
| 338 | 100 |
| 339 | 100 |
| 340 | 101 |
| 341 | 99 |
| 347 | 100 |

### Test Example 2

Assessments of anti-atherosclerotic effects and anti-obesity effects on atherogenic diet fed apoE-deficient mice were performed by the method described below.

Male apoE-deficient mice of 11-13 week-age (Jacson Lab) conditioned with ordinary diet (Research Diet) in separate cages were allowed to take high fat diet (Research Diet) at libitum for 14 weeks to thereby form atherosclerotic lesions in the aorta. The test compound suspended in 0.5% methyl cellulose solution was forcedly and orally administered to the mice at 10 ml/kg everyday for 15 weeks from 1 week prior to the start of the high fat diet feeding. Body weights were measured on the day of autopsy. The body weight reduction ratio of test compound-administered group was calculated taking the body weight of vehicle-administered group as 100%, to thereby obtain an index for anti-obesity effects. The aorta (from immediately above the aortic valve to the ventral branch of common iliac artery) removed under anesthesia was totally incised after removal of adipose tissue, etc. adhering to the outer membrane of the aorta, to thereby prepare an incised aorta specimen. After formalin fixing, the incised aorta specimen was subjected to staining with oil red O. The thus stained incised aorta specimen was photographed with a digital camera. Subsequently, image analysis using Image Pro program (Planetron, Inc.) was performed to determine the area of atherosclerotic lesions stained red and the total area of the incised specimen (the area of vascular inner wall). The ratio of atherosclerotic lesions (%) was calculated by dividing the area of atherosclerotic lesions by the area of vascular inner wall. The reduction ratio of the atherosclerotic lesion area (%) in test compound-administered group was calculated taking the area of atherosclerotic lesions in vehicle-administered group as 100%, to thereby obtain an index for anti-atherosclerotic effects. All the measurement of the ratio of atherosclerotic lesions was performed in a blind fashion.

The results are shown in Table 4.

TABLE 4

| | | | Reduction in |
|---|---|---|---|
| Example | Dose | Reduction in body | atherosclerotic |
| No. | (mg/kg/day, p.o.) | weight (%) | lesion area (%) |
| 274 | 10 | 8 | 26 |
| 297 | 10 | 8 | 26 |
| 326 | 10 | 11 | 41 |

As is clear from Table 4, the compound of the present invention demonstrated excellent anti-atherosclerotic effects and anti-obesity effects.

### Test Example 3

Anti-diabetic effects in ob/ob mice, a type 2 diabetic model were evaluated as describe next.

Male 9-week-old ob/ob mice (Charls river) habituated for a week were housed individually and freely fed a normal chow (CE-2, Japan Clea). The test compound was suspended in 0.5% methylcellulose solution. Vehicle (0.5% methylcellulose) or the compound was orally administered (5 mL/kg) once a day for 6 weeks. At the end of the treatment, bloods were collected from tail vein. The levels of glycosylated hemoglobin were measured as an index of diabetes severity using automated GHb analyzer (TOSOH Corporation).

TABLE 5

| | Dose | Glycosylated hemoglobin |
|---|---|---|
| Group | (mg/kg/day, p.o.) | (%) |
| Vehicle | — | 7.1 |
| Example 326 | 10 | 6.0 |

As clearly shown in Table 5, this compound showed superior anti-diabetic effects.

### INDUSTRIAL APPLICABILITY

The compound of the present invention has a delta-5-desaturase inhibitory effect and is useful in preventing and/or treating atherosclerosis, atherothrombosis, diabetes, obesity, asthma, fever, pain, cancer, rheumatism, osteoarthritis, atopic dermatitis and the like.

1. A compound represented by the formula (I): 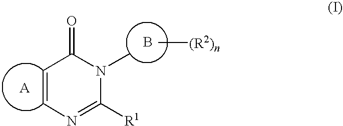
wherein:
R¹ is a hydrogen atom, a substituted or unsubstituted C₁₋₆ alkyl, a substituted or unsubstituted C₃₋₈ cycloalkyl, a substituted or unsubstituted amino, —OR′, —SR′, —SOR″ or —SO₂R″ wherein R′ is a hydrogen atom, a substituted or unsubstituted C₁₋₆ alkyl, a substituted or unsubstituted C₃₋₆ cycloalkyl, or a substituted or unsubstituted cyclic group; and R″ is a substituted or unsubstituted C₁₋₆ alkyl, a substituted or unsubstituted C₃₋₆ cycloalkyl, or a substituted or unsubstituted cyclic group;
R² is a hydrogen atom, a halogen atom, a substituted or unsubstituted C₁₋₆ alkyl, or a substituted or unsubstituted C₁₋₆ alkoxy;
n is an integer from 1 to 5;
a condensed ring including Ring A is a ring represented by any of the formulae: 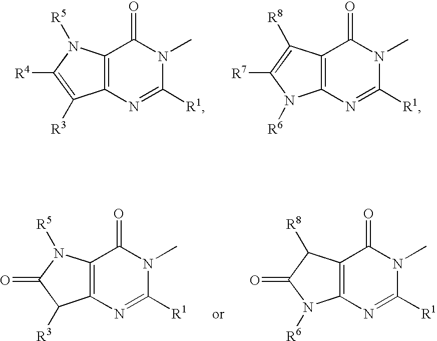
wherein:
R³ is a hydrogen atom, a substituted or unsubstituted C₁₋₆ alkyl, or a substituted or unsubstituted C₃₋₈ cycloalkyl;
R⁴ is a hydrogen atom, a halogen atom, a hydroxy, a substituted or unsubstituted C₁₋₆ alkyl, or a substituted or unsubstituted C₁₋₆ alkoxy;
R⁵ is a hydrogen atom, or a substituted or unsubstituted C₁₋₆ alkyl;
R⁶ is a hydrogen atom, a substituted or unsubstituted C₁₋₆ alkyl, or a substituted or unsubstituted C₃₋₈ cycloalkyl;
R⁷ is a hydrogen atom, a halogen atom, a substituted or unsubstituted hydroxy, a C₂₋₆ alkyl, a substituted C₁₋₆ alkyl, or a substituted or unsubstituted C₁₋₆ alkoxy; and
R⁸ is a hydrogen atom or a halogen atom; and
Ring B is a 5- or 6-membered ring, with proviso that when R⁴ is a hydrogen atom, a halogen atom, a substituted or unsubstituted C₁₋₆ alkyl or a substituted or unsubstituted C₁₋₆ alkoxy, or when R⁷ is a hydrogen atom, a halogen atom, a substituted hydroxy, a C₂₋₆ alkyl, a substituted C₁₋₆ alkyl or a substituted or unsubstituted C₁₋₆ alkoxy, Ring B is a ring represented by the formula: 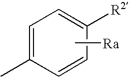
wherein:
R^{2′} is a substituted or unsubstituted C₁₋₆ alkyl or a substituted or unsubstituted C₁₋₆ alkoxy; and
Ra is a hydrogen atom, a halogen atom, a substituted or unsubstituted C₁₋₆ alkyl, or a substituted or unsubstituted C₁₋₆ alkoxy, or a salt thereof.

2. The compound according to claim 1, wherein the compound is represented by the formula (I): 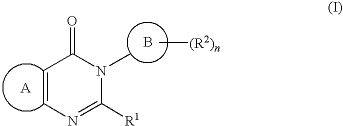
wherein:
R¹ is a hydrogen atom, a substituted or unsubstituted C₁₋₆ alkyl, a substituted or unsubstituted C₃₋₈ cycloalkyl, a substituted or unsubstituted amino, —OR′, —SR′, —SOR″ or —SO₂R″ wherein R′ is a hydrogen atom, a substituted or unsubstituted C₁₋₆ alkyl, a substituted or unsubstituted C₃₋₆ cycloalkyl, or a substituted or unsubstituted cyclic group; and R″ is a substituted or unsubstituted C₁₋₆ alkyl, a substituted or unsubstituted C₃₋₆ cycloalkyl, or a substituted or unsubstituted cyclic group;
R² is a hydrogen atom, a halogen atom, a substituted or unsubstituted C₁₋₆ alkyl, or a substituted or unsubstituted C₁₋₆ alkoxy;
n is an integer from 1 to 5;
a condensed ring including Ring A is a ring represented by any of the following formulae: 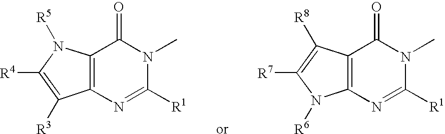
wherein:
R³ is a hydrogen atom, a substituted or unsubstituted C₁₋₆ alkyl, or a substituted or unsubstituted C₃₋₈ cycloalkyl;
R⁴ is a hydrogen atom, a halogen atom, a hydroxy, a substituted or unsubstituted C₁₋₆ alkyl, or a substituted or unsubstituted C₁₋₆ alkoxy;
R⁵ is a hydrogen atom or a substituted or unsubstituted C₁₋₆ alkyl;
R⁶ is a hydrogen atom, a substituted or unsubstituted C₁₋₆ alkyl, or a substituted or unsubstituted C₃₋₈ cycloalkyl;
R⁷ is a hydrogen atom, a halogen atom, a substituted or unsubstituted hydroxy, a C₂₋₆ alkyl, a substituted C₁₋₆ alkyl, or a substituted or unsubstituted C₁₋₆ alkoxy; and
R⁸ is a hydrogen atom or a halogen atom; and
Ring B is a 5- or 6-membered ring, with proviso that when R⁴ is a hydrogen atom, a halogen atom, a substituted or unsubstituted C₁₋₆ alkyl or a substituted or unsubstituted C₁₋₆ alkoxy, or when R⁷ is a hydrogen atom, a halogen atom, a substituted hydroxy, a C₂₋₆ alkyl, a substituted C₁₋₆ alkyl or a substituted or unsubstituted C₁₋₆ alkoxy, Ring B is a ring represented by the formula: 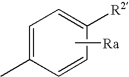
wherein:
R^{2′} is a substituted or unsubstituted C₁₋₆ alkyl or a substituted or unsubstituted C₁₋₆ alkoxy; and
Ra is a hydrogen atom, a halogen atom, a substituted or unsubstituted C₁₋₆ alkyl, or a substituted or unsubstituted C₁₋₆ alkoxy.

3. The compound according to claim 1, wherein Ring B is a ring represented by the formula: 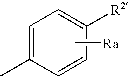
wherein R^{2′} and Ra have the same meanings as those in claim 1.

4. The compound according to claim 1, wherein the compound is represented by the formula (I): 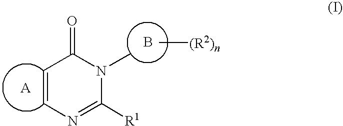
wherein:
R¹ is a hydrogen atom, a substituted or unsubstituted C₁₋₆ alkyl, a substituted or unsubstituted C₃₋₈ cycloalkyl, a substituted or unsubstituted amino, —OR′, —SR′, —SOR″ or —SO₂R″ wherein R′ is a hydrogen atom, a substituted or unsubstituted C₁₋₆ alkyl, a substituted or unsubstituted C₃₋₆ cycloalkyl, or a substituted or unsubstituted cyclic group; and R″ is a substituted or unsubstituted C₁₋₆ alkyl, a substituted or unsubstituted C₃₋₆ cycloalkyl, or a substituted or unsubstituted cyclic group;
R² is a hydrogen atom, a halogen atom, a substituted or unsubstituted C₁₋₆ alkyl, or a substituted or unsubstituted C₁₋₆ alkoxy;
n is an integer from 1 to 5;
a condensed ring including Ring A is a ring represented by any of the following formulae: 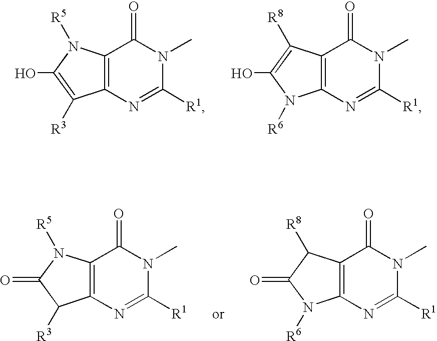
wherein:
R³ is a hydrogen atom, a substituted or unsubstituted C₁₋₆ alkyl, or a substituted or unsubstituted C₃₋₈ cycloalkyl;
R⁵ is a hydrogen atom or a substituted or unsubstituted C₁₋₆ alkyl;
R⁶ is a hydrogen atom, a substituted or unsubstituted C₁₋₆ alkyl, or a substituted or unsubstituted C₃₋₈ cycloalkyl; and
R⁸ is a hydrogen atom or a halogen atom; and
Ring B is a 5- or 6-membered ring.

5. The compound according to claim 4, wherein Ring B is a ring represented by the formula: 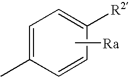
wherein:
R^{2′} is a C₁₋₆ alkoxy which may be substituted with 1 to 9 substituents selected from the group consisting of a halogen atom and a C₃₋₆ cycloalkyl; and
Ra is a hydrogen atom or a halogen atom.

6. The compound according to claim 4, wherein R¹ is —OR′ or —SR′ wherein R′ is a C₁₋₆ alkyl, a C₃₋₆ cycloalkyl or a C₆₋₁₄ aryl, each of which may be substituted with 1 to 5 substituents selected from the group consisting of (a) a halogen atom, (b) a C₁₋₆ alkoxy which may be substituted with 1 to 3 C₁₋₆ alkoxy, (c) a C₃₋₆ cycloalkyl and (d) a C₁₋₆ alkylsulfonyl.

7. The compound according to claim 4, wherein R² is (a) a hydrogen atom, (b) a halogen atom or (c) a C₁₋₆ alkoxy which may be substituted with 1 to 9 substituents selected from the group consisting of a halogen atom and a C₃₋₆ cycloalkyl; and n is 1.

8. The compound according to claim 4, wherein the condensed ring including Ring A is a ring represented by any of the following formulae: 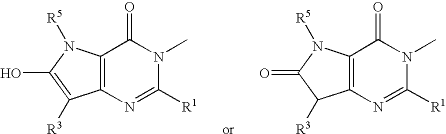
wherein R¹, R³ and R⁵ have the same meanings as those in claim 4.

9. The compound according to claim 8, wherein R³ is a hydrogen atom, a C₁₋₆ alkyl or a C₃₋₈ cycloalkyl.

10. The compound according to claim 8, wherein R⁵ is a hydrogen atom.

11. The compound according to claim 4, wherein the condensed ring including Ring A is a ring represented by any of the following formulae: 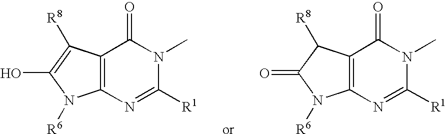
wherein R¹, R⁶ and R⁸ have the same meanings as those in claim 4.

12. The compound according to claim 11, wherein R⁶ is a hydrogen atom or a substituted or unsubstituted C₁₋₆ alkyl.

13. The compound according to claim 11, wherein R⁸ is a hydrogen atom.

14. The compound according to claim 4, wherein:
R¹ is —OR′ or —SR′ wherein R′ is a C₁₋₆ alkyl, a C₃₋₆ cycloalkyl or a C₆₋₁₄ aryl, each of which may be substituted with 1 to 5 substituents selected from the group consisting of (a) a halogen atom, (b) a C₁₋₆ alkoxy which may be substituted with 1 to 3 C₁₋₆ alkoxy, (c) a C₃₋₆ cycloalkyl and (d) a C₁₋₆ alkylsulfonyl;
the condensed ring including Ring A is a ring represented by any of the following formulae: 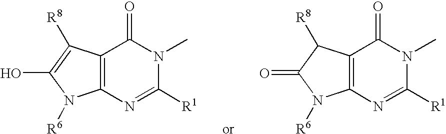
wherein:
R⁶ is a hydrogen atom, or a C₁₋₆ alkyl which may be substituted with 1 to 3 C₁₋₆ alkoxy; and
R⁸ is a hydrogen atom or a halogen atom; and
Ring B is a ring represented by the formula: 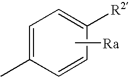
wherein:
R^{2′} is a C₁₋₆ alkoxy which may be substituted with 1 to 9 substituents selected from the group consisting of a halogen atom and a C₃₋₆ cycloalkyl; and
Ra is a hydrogen atom or a halogen atom.

15. 2-(2,2,2-Trifluoroethoxy)-3-[4-(2,2,2-trifluoroethoxy)phenyl]-5,7-dihydro-3H-pyrrolo[2,3-d]pyrimidine-4,6-dione or a salt thereof.

16. 2-(2,2,3,3,3-Pentafluoropropoxy)-3-[4-(2,2,2-trifluoroethoxy)phenyl]-5,7-dihydro-3H-pyrrolo[2,3-d]pyrimidine-4,6-dione or a salt thereof.

17. 2-[(Cyclopropylmethyl)sulfanyl]-3-[4-(2,2,2-trifluoroethoxy)phenyl]-5,7-dihydro-3H-pyrrolo[2,3-d]pyrimidine-4,6-dione or a salt thereof.

18. 2-(2,2,2-Trifluoroethoxy)-3-[4-(2,2,2-trifluoroethoxy)phenyl]-3,7-dihydro-4H-pyrrolo[2,3-d]pyrimidine-4-one or a salt thereof.

19. 2-(2,2,3,3,3-Pentafluoropropoxy)-3-[4-(2,2,2-trifluoroethoxy)phenyl]-3,7-dihydro-4H-pyrrolo[2,3-d]pyrimidine-4-one or a salt thereof.

20. 2-[(Cyclopropylmethyl)sulfanyl]-3-[4-(2,2,2-trifluoroethoxy)phenyl]-3,7-dihydro-4H-pyrrolo[2,3-d]pyrimidine-4-one or a salt thereof

21. (canceled)

22. A pharmaceutical composition comprising the compound according to claim 1 or a prodrug thereof.

23-26. (canceled)

27. A method for preventing or treating atherosclerosis in a mammal, which comprises administering an effective amount of the compound according to claim 1 or a prodrug thereof to the mammal.

28. A method for preventing or treating diabetes or obesity in a mammal, which comprises administering an effective amount of the compound according to claim 1 or a prodrug thereof to the mammal.

29-30. (canceled)
